SEQUENCE LISTING (198 sequences)

SEQ ID NO: 1 - RNA, 1236 nt, synthetic construct
  source 1..1236: mol_type = other RNA
  misc_difference 135..1134: note = This region may encompass 1-1000 nucleotides
  misc_difference 135..1134: note = a, c, u, g, unknown or other
ccgcactcgccggtcccaagcccggataaaatgggagggggcgggaaaccgcctaaccatgccgagtgcggccgcttgccatgtgtatcggtccgacatgaggatcacccatgtcggtccgatactctgatgatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtcccatcattcatggcaagtggccgcggtcggcgtggactgtagaacactgccaatgccggtcccaagcccggataaaagtggagggtacagtccacgc

SEQ ID NO: 2 - RNA, 1243 nt, synthetic construct
  source 1..1243: mol_type = other RNA
  misc_difference 142..1141: note = This region may encompass 1-1000 nucleotides
  misc_difference 142..1141: note = a, c, u, g, unknown or other
gccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtcccatcattcatggcaagtggccgcggtcggcgtggactgtagaacactgccaatgccggtcccaagcccggataaaagtggagggtacagtccacgc

SEQ ID NO: 3 - RNA, 1402 nt, synthetic construct
  source 1..1402: mol_type = other RNA
  misc_difference 301..1300: note = This region may encompass 1-1000 nucleotides
  misc_difference 301..1300: note = a, c, u, g, unknown or other
gccgcactcgccggtcccaagcccggataaaatgggagggggcgggaaaccgcctaaccatgccgagtgcggccgcttgccatgtgtatcggtccgcttaagaaaaaaaaaggggttggggatttagctcagtggtagagcgcttgcctagcaagcgcaaggccctgggttcggtcctcagctctggaaaaaaaaaaaaaaaaaaaaaaagacaaaataacaaaaagaccaaaaaaaaacaaggtaactggcacacacaacctttaaaaaaaaagttaaccggtccgatactctgatgatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtcccatcattcatggcaagtggccgcggtcggcgtggactgtagaacactgccaatgccggtcccaagcccggataaaagtggagggtacagtccacgc

SEQ ID NO: 4 - RNA, 1307 nt, synthetic construct
  source 1..1307: mol_type = other RNA
  misc_difference 148..1147: note = This region may encompass 1-1000 nucleotides
  misc_difference 148..1147: note = a, c, u, g, unknown or other
cgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncactaacctaagacaggagggccgggaaacctgcctaatccaatgacgggtaatagtggggacccatcattcatggcaagtggccgcggtcggcgtggactgtagaacactgccaatgccggtcccaagcccggataaaagtggagggtacagtccacgc

SEQ ID NO: 5 - RNA, 1273 nt, synthetic construct
  source 1..1273: mol_type = other RNA
  misc_difference 142..1141: note = This region may encompass 1-1000 nucleotides
  misc_difference 142..1141: note = a, c, u, g, unknown or other
gccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaaaaaaaaaaaaaaaagggtcccatcattcatggcaagtggccgcggtcggcgtggactgtagaacactgccaatgccggtcccaagcccggataaaagtggagggtacagtccacgc

SEQ ID NO: 6 - RNA, 60 nt, synthetic construct
  source 1..60: mol_type = other RNA
  misc_difference 1..60: note = This sequence may encompass 10-60 nucleotides
aaaaaaaaaaaaaaaaaaaaaaaaaaaaaaaaaaaaaaaaaaaaaaaaaaaaaaaaaaaa

SEQ ID NO: 7 - RNA, 30 nt, synthetic construct
  source 1..30: mol_type = other RNA
aaaaaaaaaaaaaaaaaaaaaaaaaaaaaa

SEQ ID NO: 8 - protein, 594 aa, synthetic construct
MASNFTQFVLVDNGGTGDVTVAPSNFANGVAEWISSNSRSQAYKVTCSVRQSSAQNRKYTIKVEVPKGAWRSYLNMELTIPIFSTNSDCELIVKAMQGLLKDGNPIPSAIAANSGIYGGGGSGGGGSNDFGNYNNQSSNFGPMKGGNFGGRSSGPYGGGGQYFAKPRNQGGYGGGGSLPPLERLTLGSGGSGGSEGRGSLLTCGDVEENPGPATMLEVKEASPTSIQISWVLHLRHVRYYRITYGETGGNSPVQEFTVPGSKSTATISGLKPGVDYTITVYAVTIFSAYRSAWPPISINYRTGTDYKDDDDKGSGSSRSGLLKATMASNFTQFVLVDNGGTGDVTVAPSNFANGVAEWISSNSRSQAYKVTCSVRQSSAQKRKYTIKVEVPKVATQTVGGVELPVAARRSYLNMELTIPIFATNSDCELIVKAMQGLLKDGNPIPSAIAANSGIYGAPGIHPGMMASNFTQFVLVDNGGTGDVTVAPSNFANGVAEWISSNSRSQAYKVTCSVRQSSAQKRKYTIKVEVPKVATQTVGGVELPVAARRSYLNMELTIPIFATNSDCELIVKAMQGLLKDGNPIPSAIAANSGIY

SEQ ID NO: 9 - protein, 642 aa, synthetic construct
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRGGGGSGGGGSNDFGNYNNQSSNFGPMKGGNFGGRSSGPYGGGGQYFAKPRNQGGYGGGGSLPPLERLTLGSGGSGGSEGRGSLLTCGDVEENPGPATMSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRRADPLASCGRSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRRADPLASTRDSTSGGSGGGYPYDVPDYASGGGSGGGMSATAATVPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 10 - protein, 408 aa, synthetic construct
MGKPIPNPLLGLDSTGGGGSSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRGGGGSGGGGSNDFGNYNNQSSNFGPMKGGNFGGRSSGPYGGGGQYFAKPRNQGGYGGGGSLPPLERLTLGSGGSGGSEGRGSLLTCGDVEENPGPAMDYKDDDDKGGGGSSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRGGGGSKLNPPDESGPGCMSCCVLS

SEQ ID NO: 11 - protein, 635 aa, synthetic construct
MGKPIPNPLLGLDSTGGGGSSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRGGGGSGGGGSNDFGNYNNQSSNFGPMKGGNFGGRSSGPYGGGGQYFAKPRNQGGYGGGGSLPPLERLTLGSGGSGGSEGRGSLLTCGDVEENPGPAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGGGSSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRGGGGSKLNPPDESGPGCMSCCVLS

SEQ ID NO: 12 - protein, 235 aa, synthetic construct
VSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 13 - protein, 196 aa, synthetic construct
SKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRGGGGSGGGGSNDFGNYNNQSSNFGPMKGGNFGGRSSGPYGGGGQYFAKPRNQGGYGGGGSLPPLERLTL

SEQ ID NO: 14 - protein, 151 aa, synthetic construct
SKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGRGGGGSKLNPPDESGPGCMSCCVLS

SEQ ID NO: 15 - protein, 186 aa, synthetic construct
MASNFTQFVLVDNGGTGDVTVAPSNFANGVAEWISSNSRSQAYKVTCSVRQSSAQNRKYTIKVEVPKGAWRSYLNMELTIPIFSTNSDCELIVKAMQGLLKDGNPIPSAIAANSGIYGGGGSGGGGSNDFGNYNNQSSNFGPMKGGNFGGRSSGPYGGGGQYFAKPRNQGGYGGGGSLPPLERLTL

SEQ ID NO: 16 - protein, 396 aa, Homo sapiens
MELDHRTSGGLHAYPGPRGGQVAKPNVILQIGKCRAEMLEHVRRTHRHLLAEVSKQVERELKGLHRSVGKLESNLDGYVPTSDSQRWKKSIKACLCRCQETIANLERWVKREMHVWREVFYRLERWADRLESTGGKYPVGSESARHTVSVGVGGPESYCHEADGYDYTVSPYAITPPPAAGELPGQEPAEAQQYQPWVPGEDGQPSPGVDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDADEEEIIQYVVGTLQPKLKRFLRHPLPKTLEQLIQRGMEVQDDLEQAAEPAGPHLPVEDEAETLTPAPNSESVASDRTQPE

SEQ ID NO: 17 - DNA, 1191 bp, Homo sapiens
  source 1..1191: mol_type = genomic DNA
atggagctggaccaccggaccagcggcgggctccacgcctaccccgggccgcggggcgggcaggtggccaagcccaacgtgatcctgcagatcgggaagtgccgggccgagatgctggagcacgtgcggcggacgcaccggcacctgctggccgaggtgtccaagcaggtggagcgcgagctgaaggggctgcaccggtcggtcgggaagctggagagcaacctggacggctacgtgcccacgagcgactcgcagcgctggaagaagtccatcaaggcctgcctgtgccgctgccaggagaccatcgccaacctggagcgctgggtcaagcgcgagatgcacgtgtggcgcgaggtgttctaccgcctggagcgctgggccgaccgcctggagtccacgggcggcaagtacccggtgggcagcgagtcagcccgccacaccgtttccgtgggcgtggggggtcccgagagctactgccacgaggcagacggctacgactacaccgtcagcccctacgccatcaccccgcccccagccgctggcgagctgcccgggcaggagcccgccgaggcccagcagtaccagccgtgggtccccggcgaggacgggcagcccagccccggcgtggacacgcagatcttcgaggaccctcgagagttcctgagccacctagaggagtacttgcggcaggtgggcggctctgaggagtactggctgtcccagatccagaatcacatgaacgggccggccaagaagtggtgggagttcaagcagggctccgtgaagaactgggtggagttcaagaaggagttcctgcagtacagcgagggcacgctgtcccgagaggccatccagcgcgagctggacctgccgcagaagcagggcgagccgctggaccagttcctgtggcgcaagcgggacctgtaccagacgctctacgtggacgcggacgaggaggagatcatccagtacgtggtgggcaccctgcagcccaagctcaagcgtttcctgcgccaccccctgcccaagaccctggagcagctcatccagaggggcatggaggtgcaggatgacctggagcaggcggccgagccggccggcccccacctcccggtggaggatgaggcggagaccctcacgcccgcccccaacagcgagtccgtggccagtgaccggacccagcccgagtag

SEQ ID NO: 18 - DNA, 154 bp, synthetic construct
  source 1..154: mol_type = other DNA
ggggttggggatttagctcagtggtagagcgcttgcctagcaagcgcaaggccctgggttcggtcctcagctctggaaaaaaaaaaaaaaaaaaaaaaagacaaaataacaaaaagaccaaaaaaaaacaaggtaactggcacacacaaccttt

SEQ ID NO: 19 - DNA, 200 bp, synthetic construct
  source 1..200: mol_type = other DNA
ggccgggcgcggtggctcacgcctgtaatcccagctctcagggaggctaagaggcgggaggatagcttgagcccaggagttcgagacctgcctgggcaatatagcgagaccccgttctccagaaaaaggaaaaaaaaaaacaaaagacaaaaaaaaaataagcgtaacttccctcaaagcaacaaccccccccccccttt

SEQ ID NO: 20 - DNA, 17 bp, synthetic construct
  source 1..17: mol_type = other DNA
ggccctgaaaaagggcc

SEQ ID NO: 21 - protein, 427 aa, Homo sapiens
MAEQDVENDLLDYDEEEEPQAPQESTPAPPKKDIKGSYVSIHSSGFRDFLLKPELLRAIVDCGFEHPSEVQHECIPQAILGMDVLCQAKSGMGKTAVFVLATLQQIEPVNGQVTVLVMCHTRELAFQISKEYERFSKYMPSVKVSVFFGGLSIKKDEEVLKKNCPHVVVGTPGRILALVRNRSFSLKNVKHFVLDECDKMLEQLDMRRDVQEIFRLTPHEKQCMMFSATLSKDIRPVCRKFMQDPMEVFVDDETKLTLHGLQQYYVKLKDSEKNRKLFDLLDVLEFNQVIIFVKSVQRCMALAQLLVEQNFPAIAIHRGMAQEERLSRYQQFKDFQRRILVATNLFGRGMDIERVNIVFNYDMPEDSDTYLHRVARAGRFGTKGLAITFVSDENDAKILNDVQDRFEVNVAELPEEIDISTYIEQSR

SEQ ID NO: 22 - DNA, 1278 bp, Homo sapiens
  source 1..1278: mol_type = genomic DNA
gcagaacaggatgtggaaaacgatcttttggattacgatgaagaggaagagccccaggctcctcaagagagcacaccagctccccctaagaaagacatcaagggatcctacgtttccatccacagctctggcttccgggactttctgctgaagccggagctcctgcgggccatcgtggactgtggctttgagcatccttctgaggtccagcatgagtgcattccccaggccatcctgggcatggacgtcctgtgccaggccaagtccgggatgggcaagacagcggtcttcgtgctggccaccctacagcagattgagcctgtcaacggacaggtgacggtcctggtcatgtgccacacgagggagctggccttccagatcagcaaggaatatgagcgcttttccaagtacatgcccagcgtcaaggtgtctgtgttcttcggtggtctctccatcaagaaggatgaagaagtgttgaagaagaactgtccccatgtcgtggtggggaccccgggccgcatcctggcgctcgtgcggaataggagcttcagcctaaagaatgtgaagcactttgtgctggacgagtgtgacaagatgctggagcagctggacatgcggcgggatgtgcaggagatcttccgcctgacaccacacgagaagcagtgcatgatgttcagcgccaccctgagcaaggacatccggcctgtgtgcaggaagttcatgcaggatccaatggaggtgtttgtggacgacgagaccaagctcacgctgcacggcctgcagcagtactacgtcaaactcaaagacagtgagaagaaccgcaagctctttgatctcttggatgtgctggagtttaaccaggtgataatcttcgtcaagtcagtgcagcgctgcatggccctggcccagctcctcgtggagcagaacttcccggccatcgccatccaccggggcatggcccaggaggagcgcctgtcacgctatcagcagttcaaggatttccagcggcggatcctggtggccaccaatctgtttggccgggggatggacatcgagcgagtcaacatcgtctttaactacgacatgcctgaggactcggacacctacctgcaccgggtggcccgggcgggtcgctttggcaccaaaggcctagccatcacttttgtgtctgacgagaatgatgccaaaatcctcaatgacgtccaggaccggtttgaagttaatgtggcagaacttccagaggaaatcgacatctccacatacatcgagcagagccgg

SEQ ID NO: 23 - protein, 19 aa, synthetic construct
KLNPPDESGPGCMSCCVLS

SEQ ID NO: 24 - DNA, 57 bp, unidentified
  source 1..57: mol_type = unassigned DNA
aagctgaaccctcctgatgagagtggccccggctgcatgagctgctgtgtgctctcc

SEQ ID NO: 25 - DNA, 58 bp, synthetic construct
  source 1..58: mol_type = other DNA
cactaacctaagacaggagggccgggaaacctgcctaatccaatgacgggtaatagtg

SEQ ID NO: 26 - protein, 90 aa, synthetic construct
MLEVKEASPTSIQISWGKYKVMVRYYRITYGETGGNSPVQEFTVPGSKSTATISSLKPGVDYTITVYAVTIDHWNYQDPIPISINYRTGS

SEQ ID NO: 27 - DNA, 270 bp, synthetic construct
  source 1..270: mol_type = other DNA
atgctcgaagtcaaggaagcatcaccaaccagcatccagatcagctggggcaagtacaaggtcatggttcgctactaccgcatcacctacggtgaaactggtggcaatagccctgtccaggaattcaccgtgcctggcagcaagtccactgctaccatcagcagcctgaaacctggtgtcgactataccatcacggtgtacgccgtcacgatcgaccactggaactaccaggacccgatcccgatctccatcaactaccgcaccggatcc

SEQ ID NO: 28 - protein, 366 aa, Rattus norvegicus
MGEQPIFSTRAHVFQIDPNTKKNWVPTSKHAVTVSYFYDSTRNVYRIISLDGSKAIINSTITPNMTFTKTSQKFGQWADSRANTVYGLGFSSEHHLSKFAEKFQEFKEAARLAKEKSQEKMELTSTPSQESAGGDLQSPLTPESINGTDDERTPDVTQNSEPRAEPAQNALPFSHSAGDRTQGLSHASSAISKHWEAELATLKGNNAKLTAALLESTANVKQWKQQLAAYQEEAERLHKRVTELECVSSQANAVHSHKTELSQTVQELEETLKVKEEEIERLKQEIDNARELQEQRDSLTQKLQEVEIRNKDLEGQLSELEQRLEKSQSEQDAFRSNLKTLLEILDGKIFELTELRDNLAKLLECS

SEQ ID NO: 29 - DNA, 1101 bp, synthetic construct
  source 1..1101: mol_type = other DNA
atgggggaacaacctatcttcagcactcgagctcatgtcttccagatcgacccaaacacaaagaagaactgggtacccaccagcaagcatgcagttactgtgtcttatttctatgacagcacaaggaatgtgtataggataatcagtttagacggctcaaaggcaataataaatagcaccatcactccaaacatgacatttactaaaacatctcaaaagtttggccaatgggctgatagccgggcaaacactgtttatggactgggattctcctctgagcatcatctctcaaaatttgcagaaaagtttcaggaatttaaagaagctgctcggctggcaaaggagaagtcgcaggagaagatggaactgaccagtaccccttcacaggaatcagcaggaggagatcttcagtctcctttaacaccagaaagtatcaatgggacagatgatgagagaacacccgatgtgacacagaactcagagccaagggctgagccagctcagaatgcattgccattttcacatagtgccggggatcgaacccagggcctctctcatgctagttcagccatcagcaaacactgggaggctgaactagccacgctcaaggggaacaatgccaagctcaccgcagcgctgctggagtccactgccaacgtgaagcagtggaagcaacagctggctgcctaccaggaggaggcagagcggctgcacaagcgggtcacggagctggaatgtgttagtagtcaagcaaacgcggtgcacagccacaagacagagctgagtcagacagtgcaggagctggaagagaccctaaaagtaaaggaagaggaaatagaaagattaaaacaagaaattgataacgccagagaacttcaagaacagagggactctttgactcagaaactacaggaagttgagattcgaaataaagacctggaggggcagctgtcggagctggagcagcgcctggagaagagccagagcgagcaggacgctttccgcagtaacctgaagactctcctagagattctggacgggaaaatatttgaactaacagaattgcgggataatttggccaagctactagaatgcagctaa

SEQ ID NO: 30 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 31 - protein, 21 aa, synthetic construct
DAQTRRRERRAEKQAQWKAAN

SEQ ID NO: 32 - DNA, 63 bp, synthetic construct
  source 1..63: mol_type = other DNA
gacgcacaaacacgacgacgtgagcgtcgcgctgagaaacaagctcaatggaaagctgcaaac

SEQ ID NO: 33 - protein, 45 aa, synthetic construct
NDFGNYNNQSSNFGPMKGGNFGGRSSGPYGGGGQYFAKPRNQGGY

SEQ ID NO: 34 - DNA, 135 bp, synthetic construct
  source 1..135: mol_type = other DNA
aatgattttggcaattacaacaatcagtcttccaattttgggccgatgaagggaggaaactttggaggcaggagctctggcccttatggtggtggaggccagtactttgctaaaccacggaaccaaggtggctat

SEQ ID NO: 35 - protein, 117 aa, synthetic construct
MASNFTQFVLVDNGGTGDVTVAPSNFANGVAEWISSNSRSQAYKVTCSVRQSSAQNRKYTIKVEVPKGAWRSYLNMELTIPIFSTNSDCELIVKAMQGLLKDGNPIPSAIAANSGIY

SEQ ID NO: 36 - DNA, 351 bp, synthetic construct
  source 1..351: mol_type = other DNA
atggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaaggcgcatggcgttcgtacttaaatatggaactaaccattccaattttctccacgaactccgactgcgagcttattgttaaggcaatgcaaggtctcctaaaagatggaaacccgattccctcagcaatcgcagcaaactccggcatctac

SEQ ID NO: 37 - DNA, 19 bp, synthetic construct
  source 1..19: mol_type = other DNA
acatgaggatcacccatgt

SEQ ID NO: 38 - protein, 128 aa, synthetic construct
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 39 - DNA, 381 bp, synthetic construct
  source 1..381: mol_type = other DNA
tccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgt

SEQ ID NO: 40 - DNA, 25 bp, synthetic construct
  source 1..25: mol_type = other DNA
ggagcagacgatatggcgtcgctcc

SEQ ID NO: 41 - DNA, 25 bp, synthetic construct
  source 1..25: mol_type = other DNA
ccagcagagcatatgggctcgctgg

SEQ ID NO: 42 - protein, 90 aa, synthetic construct
MLEVKEASPTSIQISWVLHLRHVRYYRITYGETGGNSPVQEFTVPGSKSTATISGLKPGVDYTITVYAVTIFSAYRSAWPPISINYRTGT

SEQ ID NO: 43 - DNA, 270 bp, synthetic construct
  source 1..270: mol_type = other DNA
atgctcgaagtcaaggaagcatcaccaaccagcatccagatcagctgggtgctccacttgcgccacgttcgctactaccgcatcacctacggtgaaactggtggcaatagccctgtccaggaattcaccgtgcctggcagcaagtccactgctaccatcagcggcctgaaacctggtgtcgactataccatcacggtgtacgccgtcacgatcttcagcgcctaccgctccgcctggccgccgatctccatcaactaccgcaccggaacc

SEQ ID NO: 44 - protein, 408 aa, unidentified
MNYELLTTENAPVKMWTKGVPVEADARQQLINTAKMPFIFKHIAVMPDVHLGKGSTIGSVIPTKGAIIPAAVGVDIGCGMNALRTALTAEDLPENLAELRQAIETAVPHGRTTGRCKRDKGAWENPPVNVDAKWAELEAGYQWLTQKYPRFLNTNNYKHLGTLGTGNHFIEICLDESDQVWIMLHSGSRGIGNAIGTYFIDLAQKEMQETLETLPSRDLAYFMEGTEYFDDYLKAVAWAQLFASLNRDAMMENVVTALQSITQKTVRQPQTLAMEEINCHHNYVQKEQHFGEEIYVTRKGAVSARAGQYGIIPGSMGAKSFIVRGLGNEESFCSCSHGAGRVMSRTKAKKLFSVEDQIRATAHVECRKDAEVIDEIPMAYKDIDAVMAAQSDLVEVIYTLRQVVCVKG

SEQ ID NO: 45 - DNA, 1221 bp, synthetic construct
  source 1..1221: mol_type = other DNA
aactatgagcttttgaccactgagaacgctcctgttaagatgtggacaaaaggcgtgcctgtagaggccgacgctcggcagcaactcattaacaccgccaagatgccctttattttcaagcatattgccgtgatgcctgatgtccatcttggtaagggttcaacaatcgggagcgtcatccctaccaagggtgccatcattccagccgccgtaggagtagatattggatgcggcatgaacgcacttagaacagctctgaccgccgaggatcttcccgagaacctcgctgaactgcgacaggcaatcgagacagcagttcctcacggcagaaccacaggcaggtgtaagagagataagggcgcatgggaaaacccccccgtgaatgtcgacgcaaaatgggcagagttggaagctgggtatcaatggctgacccaaaagtacccacggttcctcaatactaataactataagcaccttgggacactcggaaccggcaaccacttcatagaaatatgcctggacgagtcagatcaagtttggataatgctccactctggttcacggggcattggcaacgctataggaacatactttatagacctggcccagaaagagatgcaagaaacattggaaactctcccaagtagggacctcgcttacttcatggagggaactgagtatttcgatgattatctgaaagccgtagcatgggcacagttgttcgcctccttgaatagggatgcaatgatggagaatgtcgtcactgctcttcaaagtatcacccaaaaaacagtacgccaacctcagactctggcaatggaagagatcaactgtcatcataactacgtacaaaaggaacaacacttcggcgaagagatctatgttacccggaaaggggccgtctcagctagggcaggccaatacggcataatccctggctctatgggtgcaaaaagcttcatagttcgaggccttgggaacgaggagagcttttgtagctgtagccacggggctggtcgggtgatgtcccggactaaagctaaaaaattgttctctgttgaggaccaaatacgggctaccgcacacgtagaatgccggaaggacgccgaggtcatcgacgaaatccctatggcctacaaggacattgacgcagttatggccgcacagtctgacctggtggaagttatatatacactgaggcaagtagtatgtgtgaaggga

SEQ ID NO: 46 - protein, 307 aa, Rattus norvegicus
MDVVNQLVAGGQFRVVKEPLGFVKVLQWVFAIFAFATCGSYTGELRLSVECANKTESALNIEVEFEYPFRLHQVYFDAPSCVKGGTTKIFLVGDYSSSAEFFVTVAVFAFLYSMGALATYIFLQNKYRENNKGPMMDFLATAVFAFMWLVSSSAWAKGLSDVKMATDPENIIKEMPMCRQTGNTCKELRDPVTSGLNTSVVFGFLNLVLWVGNLWFVFKETGWAAPFMRAPPGAPEKQPAPGDAYGDAGYGQGPGGYGPQDSYGPQGGYQPDYGQPASGGGGYGPQGDYGQQGYGQQGAPTSFSNQM

SEQ ID NO: 47 - DNA, 924 bp, Rattus norvegicus
  source 1..924: mol_type = genomic DNA
atggacgtggtgaatcagctggtggctgggggtcagttccgggtggtcaaggagccccttggcttcgtgaaggtgctgcagtgggtctttgccatcttcgcctttgctacgtgtggcagctacaccggggagcttcggctgagcgtggagtgtgccaacaagacggagagtgccctcaacatcgaagttgaattcgagtaccccttcaggctgcaccaagtgtactttgatgcaccctcctgcgtcaaagggggcactaccaagatcttcctggttggggactactcctcgtcggctgaattctttgtcaccgtggctgtgtttgccttcctctactccatgggggccctggccacctacatcttcctgcagaacaagtaccgagagaacaacaaagggcctatgatggactttctggctacagccgtgttcgctttcatgtggctagttagttcatcagcctgggccaaaggcctgtccgatgtgaagatggccacggacccagagaacattatcaaggagatgcccatgtgccgccagacagggaacacatgcaaggaactgagggaccctgtgacttcaggactcaacacctcagtggtgtttggcttcctgaacctggtgctctgggttggcaacttatggttcgtgttcaaggagacaggctgggcagccccattcatgcgcgcacctccaggcgccccggaaaagcaaccagcacctggcgatgcctacggcgatgcgggctacgggcagggccccggaggctatgggccccaggactcctacgggcctcagggtggttatcaacccgattacgggcagccagccagcggtggcggtggctacgggcctcagggcgactatgggcagcaaggctatggccaacagggtgcgcccacctccttctccaatcagatgtaa

SEQ ID NO: 48 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 49 - protein, 116 aa, unidentified
MSATAATVPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 50 - DNA, 348 bp, synthetic construct
  source 1..348: mol_type = other DNA
atgtcggctaccgctgccaccgtcccgcctgccgccccggccggcgagggtggcccccctgcacctcctccaaaccttactagtaacaggagactgcagcagacccaggcccaggtggatgaggtggtggacatcatgagggtgaatgtggacaaggtcctggagcgggaccagaagttgtcggagctggatgaccgtgcagatgccctccaggcaggggcctcccagtttgaaacaagtgcagccaagctcaagcgcaaatactggtggaaaaacctcaagatgatgatcatcttgggagtgatctgcgccatcatcctcatcatcatcatcgtttacttcagcact

SEQ ID NO: 51 - protein, 10 aa, synthetic construct
  VARIANT 1..10: note = This sequence may encompass 1-2 GGGGS repeating units
GGGGSGGGGS

SEQ ID NO: 52 - DNA, 504 bp, synthetic construct
  source 1..504: mol_type = other DNA
gccaccatggggtcttcaaaatctaaaccaaaggaccccagccagcgcggcggaggtggttctgacgcacaaacacgacgacgtgagcgtcgcgctgagaaacaagctcaatggaaagctgcaaacggcggaggtggttctgattacaaggatgacgacgataagtaagaattctgcagatatcgctcgctttcttgctgtccaatttctattaaaggttcctttgttccctaagctcgaccaaaggttcctttgtggccctgaaaaagggccaaattggtggctggtgtggctaatgccctatggccctgaaaaagggccactggaggatattcatgcactcgaccaaaggttcctttgtggccctgaaaaagggccaaattggtggctggtgtggctaatgccctatggccctgaaaaagggccactggaggatattcatgcactcgagtactaaactgggggatattatgaagggccttgagcatctggattctgcctaat

SEQ ID NO: 53 - DNA, 552 bp, synthetic construct
  source 1..552: mol_type = other DNA
gccaccatgctgtgctgcatcagaagaactaaaccggttgagaagaatgaagaggccgatcaggagctgcagtcgacggtgccgcgggcccgggatccaccggtcgccaccgacgcacaaacacgacgacgtgagcgtcgcgctgagaaacaagctcaatggaaagctgcaaacggcggaggtggttctgattacaaggatgacgacgataagtaagaattctgcagatatcgctcgctttcttgctgtccaatttctattaaaggttcctttgttccctaagctcgaccaaaggttcctttgtggccctgaaaaagggccaaattggtggctggtgtggctaatgccctatggccctgaaaaagggccactggaggatattcatgcactcgaccaaaggttcctttgtggccctgaaaaagggccaaattggtggctggtgtggctaatgccctatggccctgaaaaagggccactggaggatattcatgcactcgagtactaaactgggggatattatgaagggccttgagcatctggattctgcctaat

SEQ ID NO: 54 - DNA, 522 bp, synthetic construct
  source 1..522: mol_type = other DNA
gccaccatggattacaaggatgacgacgataagggcggaggtggttctgacgcacaaacacgacgacgtgagcgtcgcgctgagaaacaagctcaatggaaagctgcaaacggcggaggtggttctaagctgaaccctcctgatgagagtggccccggctgcatgagctgctgtgtgctctcctaagaattctgcagatatcgctcgctttcttgctgtccaatttctattaaaggttcctttgttccctaagctcgaccaaaggttcctttgtggccctgaaaaagggccaaattggtggctggtgtggctaatgccctatggccctgaaaaagggccactggaggatattcatgcactcgaccaaaggttcctttgtggccctgaaaaagggccaaattggtggctggtgtggctaatgccctatggccctgaaaaagggccactggaggatattcatgcactcgagtactaaactgggggatattatgaagggccttgagcatctggattctgcctaat

SEQ ID NO: 55 - DNA, 704 bp, synthetic construct
  source 1..704: mol_type = other DNA
gccaccatggattacaaggatgacgacgataagggcggaggtggttctgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaaggcgcatggcgttcgtacttaaatatggaactaaccattccaattttctccacgaactccgactgcgagcttattgttaaggcaatgcaaggtctcctaaaagatggaaacccgattccctcagcaatcgcagcaaactccggcatctacggcggaggtggttctaagctgaaccctcctgatgagagtggccccggctgcatgagctgctgtgtgctctcctaagaattctgcagatatccctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattaga

SEQ ID NO: 56 - DNA, 765 bp, synthetic construct
  source 1..765: mol_type = other DNA
gccaccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattctgcagatatcgatcctaaggtacctaattgcctagaaaggagcagacgatatggcgtcgctccctgcaggtcgactctagaaaccagcagagcatatgggctcgctggctgcagtattcccgggttcattagatcctaaggtacctaattgcctagaaaggagcagacgatatggcgtcgctccctgcaggtcgactctagaaaccagcagagcatatgggctcgctggctgcagtattcccgggttcattagat

SEQ ID NO: 57 - DNA, 186 bp, synthetic construct
  source 1..186: mol_type = other DNA
gccaccatggattacaaggatgacgacgataagggcggaggtggttctgacgcacaaacacgacgacgtgagcgtcgcgctgagaaacaagctcaatggaaagctgcaaacggcggaggtggttctaagctgaaccctcctgatgagagtggccccggctgcatgagctgctgtgtgctctcctaa

SEQ ID NO: 58 - DNA, 471 bp, synthetic construct
  source 1..471: mol_type = other DNA
gccaccatggattacaaggatgacgacgataagggcggaggtggttctgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaaggcgcatggcgttcgtacttaaatatggaactaaccattccaattttctccacgaactccgactgcgagcttattgttaaggcaatgcaaggtctcctaaaagatggaaacccgattccctcagcaatcgcagcaaactccggcatctacggcggaggtggttctaagctgaaccctcctgatgagagtggccccggctgcatgagctgctgtgtgctctcctaa

SEQ ID NO: 59 - DNA, 504 bp, synthetic construct
  source 1..504: mol_type = other DNA
gccaccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaa

SEQ ID NO: 60 - DNA, 621 bp, synthetic construct
  source 1..621: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccggggccctgaagaagggccccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttt

SEQ ID NO: 61 - DNA, 621 bp, synthetic construct
  source 1..621: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgacatgaggatcacccatgtcggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttt

SEQ ID NO: 62 - DNA, 627 bp, synthetic construct
  source 1..627: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttt

SEQ ID NO: 63 - DNA, 495 bp, synthetic construct
  source 1..495: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggcactcttccgtggtctggtggataaattcgttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaacgacgtcagaccacgggggagtgcccttttttt

SEQ ID NO: 64 - DNA, 407 bp, synthetic construct
  source 1..407: mol_type = other DNA
cgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatcctaggttaaggatgcaccgacgggacgttctatgaggacgaatctcccgcttataagattctataaactgtgcggtccttcaattggggtccccactaacctaagacaggagggccgggaaacctgcctaatccaatgacgggtaatagtggggacccatcattcatggcaagtggccgcggtcggcgtggactgtagaacactgccaatgccggtcccaagcccggataaaagtggagggtacagtccacgctttttt

SEQ ID NO: 65 - DNA, 657 bp, synthetic construct
  source 1..657: mol_type = other DNA
gccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgtaa

SEQ ID NO: 66 - DNA, 1425 bp, synthetic construct
  source 1..1425: mol_type = other DNA
ccatgggcaagcccatccccaaccccctgctgggcctggacagcaccggcagcggcaactatgagcttttgaccactgagaacgctcctgttaagatgtggacaaaaggcgtgcctgtagaggccgacgctcggcagcaactcattaacaccgccaagatgccctttattttcaagcatattgccgtgatgcctgatgtccatcttggtaagggttcaacaatcgggagcgtcatccctaccaagggtgccatcattccagccgccgtaggagtagatattggatgcggcatgaacgcacttagaacagctctgaccgccgaggatcttcccgagaacctcgctgaactgcgacaggcaatcgagacagcagttcctcacggcagaaccacaggcaggtgtaagagagataagggcgcatgggaaaacccccccgtgaatgtcgacgcaaaatgggcagagttggaagctgggtatcaatggctgacccaaaagtacccacggttcctcaatactaataactataagcaccttgggacactcggaaccggcaaccacttcatagaaatatgcctggacgagtcagatcaagtttggataatgctccactctggttcacggggcattggcaacgctataggaacatactttatagacctggcccagaaagagatgcaagaaacattggaaactctcccaagtagggacctcgcttacttcatggagggaactgagtatttcgatgattatctgaaagccgtagcatgggcacagttgttcgcctccttgaatagggatgcaatgatggagaatgtcgtcactgctcttcaaagtatcacccaaaaaacagtacgccaacctcagactctggcaatggaagagatcaactgtcatcataactacgtacaaaaggaacaacacttcggcgaagagatctatgttacccggaaaggggccgtctcagctagggcaggccaatacggcataatccctggctctatgggtgcaaaaagcttcatagttcgaggccttgggaacgaggagagcttttgtagctgtagccacggggctggtcgggtgatgtcccggactaaagctaaaaaattgttctctgttgaggaccaaatacgggctaccgcacacgtagaatgccggaaggacgccgaggtcatcgacgaaatccctatggcctacaaggacattgacgcagttatggccgcacagtctgacctggtggaagttatatatacactgaggcaagtagtatgtgtgaagggatctggtggttctcccaagaagaagaggaaggtggaccccaagaagaagaggaaggtggaccccaagaagaagaggaaggtgggctcaggaggagagggcagaggaagtcttctaacatgcggtgacgtggaggagaatcccggccctg

SEQ ID NO: 67 - DNA, 1401 bp, synthetic construct
  source 1..1401: mol_type = other DNA
ccatgggcaagcccatccccaaccccctgctgggcctggacagcaccggcagcggcgcagaacaggatgtggaaaacgatcttttggattacgatgaagaggaagagccccaggctcctcaagagagcacaccagctccccctaagaaagacatcaagggatcctacgtttccatccacagctctggcttccgggactttctgctgaagccggagctcctgcgggccatcgtggactgtggctttgagcatccttctgaggtccagcatgagtgcattccccaggccatcctgggcatggacgtcctgtgccaggccaagtccgggatgggcaagacagcggtcttcgtgctggccaccctacagcagattgagcctgtcaacggacaggtgacggtcctggtcatgtgccacacgagggagctggccttccagatcagcaaggaatatgagcgcttttccaagtacatgcccagcgtcaaggtgtctgtgttcttcggtggtctctccatcaagaaggatgaagaagtgttgaagaagaactgtccccatgtcgtggtggggaccccgggccgcatcctggcgctcgtgcggaataggagcttcagcctaaagaatgtgaagcactttgtgctggacgagtgtgacaagatgctggagcagctggacatgcggcgggatgtgcaggagatcttccgcctgacaccacacgagaagcagtgcatgatgttcagcgccaccctgagcaaggacatccggcctgtgtgcaggaagttcatgcaggatccaatggaggtgtttgtggacgacgagaccaagctcacgctgcacggcctgcagcagtactacgtcaaactcaaagacagtgagaagaaccgcaagctctttgatctcttggatgtgctggagtttaaccaggtgataatcttcgtcaagtcagtgcagcgctgcatggccctggcccagctcctcgtggagcagaacttcccggccatcgccatccaccggggcatggcccaggaggagcgcctgtcacgctatcagcagttcaaggatttccagcggcggatcctggtggccaccaatctgtttggccgggggatggacatcgagcgagtcaacatcgtctttaactacgacatgcctgaggactcggacacctacctgcaccgggtggcccgggcgggtcgctttggcaccaaaggcctagccatcacttttgtgtctgacgagaatgatgccaaaatcctcaatgacgtccaggaccggtttgaagttaatgtggcagaacttccagaggaaatcgacatctccacatacatcgagcagagccggtctggtggttctgagggcagaggaagtcttctaacatgcggtgacgtggaggagaatcccggccctg

SEQ ID NO: 68 - DNA, 786 bp, synthetic construct
  source 1..786: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgcttaagaaaaaaaaaggggttggggatttagctcagtggtagagcgcttgcctagcaagcgcaaggccctgggttcggtcctcagctctggaaaaaaaaaaaaaaaaaaaaaaagacaaaataacaaaaagaccaaaaaaaaacaaggtaactggcacacacaacctttaaaaaaaaagttaaccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttt

SEQ ID NO: 69 - DNA, 218 bp, synthetic construct
  source 1..218: mol_type = other DNA
aaaaaaaaaggccgggcgcggtggctcacgcctgtaatcccagctctcagggaggctaagaggcgggaggatagcttgagcccaggagttcgagacctgcctgggcaatatagcgagaccccgttctccagaaaaaggaaaaaaaaaaacaaaagacaaaaaaaaaataagcgtaacttccctcaaagcaacaacccccccccccctttaaaaaaaaa

SEQ ID NO: 70 - DNA, 53 bp, synthetic construct
  source 1..53: mol_type = other DNA
ccacttgaaagactgacactgggctcaggaggatctggtggttctgagggcag

SEQ ID NO: 71 - DNA, 31 bp, synthetic construct
  source 1..31: mol_type = other DNA
gtaatccagaggttgattatcgataagcttg

SEQ ID NO: 72 - DNA, 58 bp, synthetic construct
  source 1..58: mol_type = other DNA
cacgctttttttctagactgcagagggccctaatgattaacccgccatgctacttatc

SEQ ID NO: 73 - DNA, 43 bp, synthetic construct
  source 1..43: mol_type = other DNA
ggcttgcccatggtggcggatccaattctttgccaaaatgatg

SEQ ID NO: 74 - DNA, 71 bp, synthetic construct
  source 1..71: mol_type = other DNA
taaactgtgcggtccttcaattgaaaaaaaaaaaaaaaaaaaaaaaaaaaaaagggtcccatcattcatgg

SEQ ID NO: 75 - DNA, 71 bp, synthetic construct
  source 1..71: mol_type = other DNA
ccatgaatgatgggacccttttttttttttttttttttttttttttttcaattgaaggaccgcacagttta

SEQ ID NO: 76 - DNA, 19 bp, synthetic construct
  source 1..19: mol_type = other DNA
gcgcagtcgagaaggtacc

SEQ ID NO: 77 - DNA, 24 bp, synthetic construct
  source 1..24: mol_type = other DNA
cttgctcaccatggcagggccggg

SEQ ID NO: 78 - DNA, 26 bp, synthetic construct
  source 1..26: mol_type = other DNA
cctgccatggtgagcaagggcgagga

SEQ ID NO: 79 - DNA, 30 bp, synthetic construct
  source 1..30: mol_type = other DNA
acctccgcccttgtacagctcgtccatgcc

SEQ ID NO: 80 - DNA, 27 bp, synthetic construct
  source 1..27: mol_type = other DNA
ctgtacaagggcggaggtggttcttcc

SEQ ID NO: 81 - DNA, 28 bp, synthetic construct
  source 1..28: mol_type = other DNA
aggttgattatcgataagcttgatatcg

SEQ ID NO: 82 - DNA, 49 bp, synthetic construct
  source 1..49: mol_type = other DNA
gctttttttctagactgcagagggccctcaagtgccacctgacgtctcc

SEQ ID NO: 83 - DNA, 33 bp, synthetic construct
  source 1..33: mol_type = other DNA
cttgcccatggtggcggatccaggctggatcgg

SEQ ID NO: 84 - DNA, 6943 bp, synthetic construct
  source 1..6943: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgacatgaggatcacccatgtcggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaaggcgcatggcgttcgtacttaaatatggaactaaccattccaattttctccacgaactccgactgcgagcttattgttaaggcaatgcaaggtctcctaaaagatggaaacccgattccctcagcaatcgcagcaaactccggcatctacggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccaccatgctcgaagtcaaggaagcatcaccaaccagcatccagatcagctgggtgctccacttgcgccacgttcgctactaccgcatcacctacggtgaaactggtggcaatagccctgtccaggaattcaccgtgcctggcagcaagtccactgctaccatcagcggcctgaaacctggtgtcgactataccatcacggtgtacgccgtcacgatcttcagcgcctaccgctccgcctggccgccgatctccatcaactaccgcaccggaaccgattacaaggatgacgacgataagggtagcggctccagtagatctgggctacttaaggccaccatggccagcaacttcacccagtttgtgctggtggacaatggcgggacaggcgatgtgactgtggctccctccaacttcgccaatggggtggctgagtggatcagctccaacagtcggtcacaggcctacaaggtgacctgcagcgtgcggcagtctagtgctcagaagagaaagtacacaattaaggtggaggtgcccaaagtggccacccagacagtgggaggagtggaactgcctgtggctgctcggagatcctacctgaacatggagctgactatccctattttcgccaccaattctgactgtgaactgatcgtgaaggctatgcagggactgctgaaagatggcaaccccatcccttctgccattgccgctaatagtggaatctatggcgcccctgggattcacccagggatgatggcttctaacttcacccaatttgtgctggtcgataatgggggaaccggagatgtgacagtggccccaagtaactttgccaatggcgtggctgaatggatctcaagcaatagccggtcccaggcctacaaagtgacttgctccgtgaggcagtcctctgctcagaagcgcaaatatacaattaaggtcgaagtgccaaaagtggccactcagaccgtcggcggagtggaactgcccgtggctgctaggcgcagttacctgaatatggaactgacaatccctatttttgccactaattcagattgcgagctgattgtgaaggctatgcaggggctgctgaaagatggaaacccaatcccctcagccattgccgctaatagcggcatctacctcgagccataagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 85 - DNA, 7080 bp, synthetic construct
  source 1..7080: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccatgtccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccaccatgtccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtcgtgcggacccgctagcctcctgcggccgctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtcgtgcggacccgctagcctccacgcgtgattcgactagtggaggaagcggaggaggatatccatatgatgttccagattatgcttcaggaggaggctcaggaggaggaatgtcggctaccgctgccaccgtcccgcctgccgccccggccggcgagggtggcccccctgcacctcctccaaaccttactagtaacaggagactgcagcagacccaggcccaggtggatgaggtggtggacatcatgagggtgaatgtggacaaggtcctggagcgggaccagaagttgtcggagctggatgaccgtgcagatgccctccaggcaggggcctcccagtttgaaacaagtgcagccaagctcaagcgcaaatactggtggaaaaacctcaagatgatgatcatcttgggagtgatctgcgccatcatcctcatcatcatcatcgtttacttcagcacttgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 86 - DNA, 6030 bp, synthetic construct
  source 1..6030: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgcttaagaaaaaaaaaggggttggggatttagctcagtggtagagcgcttgcctagcaagcgcaaggccctgggttcggtcctcagctctggaaaaaaaaaaaaaaaaaaaaaaagacaaaataacaaaaagaccaaaaaaaaacaaggtaactggcacacacaacctttaaaaaaaaagttaaccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 87 - DNA, 6454 bp, synthetic construct
  source 1..6454: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatcctaggttaaggatgcaccgacgggacgttctatgaggacgaatctcccgcttataagattctataaactgtgcggtccttcaattggggtccccactaacctaagacaggagggccgggaaacctgcctaatccaatgacgggtaatagtggggacccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 88 - DNA, 7094 bp, synthetic construct
  source 1..7094: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctagggacgggacgttctgctaagatcatttctccctggggcagattctataaactgtgcggtccttcaattgaaaaaaaaaaaaaaaaaaaaaaaaaaaaaagggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 89 - DNA, 7641 bp, synthetic construct
  source 1..7641: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccatgtccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccaccatggacgtggtgaatcagctggtggctgggggtcagttccgggtggtcaaggagccccttggcttcgtgaaggtgctgcagtgggtctttgccatcttcgcctttgctacgtgtggcagctacaccggggagcttcggctgagcgtggagtgtgccaacaagacggagagtgccctcaacatcgaagttgaattcgagtaccccttcaggctgcaccaagtgtactttgatgcaccctcctgcgtcaaagggggcactaccaagatcttcctggttggggactactcctcgtcggctgaattctttgtcaccgtggctgtgtttgccttcctctactccatgggggccctggccacctacatcttcctgcagaacaagtaccgagagaacaacaaagggcctatgatggactttctggctacagccgtgttcgctttcatgtggctagttagttcatcagcctgggccaaaggcctgtccgatgtgaagatggccacggacccagagaacattatcaaggagatgcccatgtgccgccagacagggaacacatgcaaggaactgagggaccctgtgacttcaggactcaacacctcagtggtgtttggcttcctgaacctggtgctctgggttggcaacttatggttcgtgttcaaggagacaggctgggcagccccattcatgcgcgcacctccaggcgccccggaaaagcaaccagcacctggcgatgcctacggcgatgcgggctacgggcagggccccggaggctatgggccccaggactcctacgggcctcagggtggttatcaacccgattacgggcagccagccagcggtggcggtggctacgggcctcagggcgactatgggcagcaaggctatggccaacagggtgcgcccacctccttctccaatcagatgaaaaccggtggtggcggcagtggtggcggcagctatccatatgatgttccagattatgctgccaccatgtccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtcgtgcggacccgctagcctcctgcggccgctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtcgtgcggacccgctagcctccacgcgtgattgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 90 - DNA, 7122 bp, synthetic construct
  source 1..7122: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgacatgaggatcacccatgtcggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccctagccaccatggccagcaacttcacccagtttgtgctggtggacaatggcgggacaggcgatgtgactgtggctccctccaacttcgccaatggggtggctgagtggatcagctccaacagtcggtcacaggcctacaaggtgacctgcagcgtgcggcagtctagtgctcagaagagaaagtacacaattaaggtggaggtgcccaaagtggccacccagacagtgggaggagtggaactgcctgtggctgctcggagatcctacctgaacatggagctgactatccctattttcgccaccaattctgactgtgaactgatcgtgaaggctatgcagggactgctgaaagatggcaaccccatcccttctgccattgccgctaatagtggaatctatggcgcccctgggattcacccagggatgatggcttctaacttcacccaatttgtgctggtcgataatgggggaaccggagatgtgacagtggccccaagtaactttgccaatggcgtggctgaatggatctcaagcaatagccggtcccaggcctacaaagtgacttgctccgtgaggcagtcctctgctcagaagcgcaaatatacaattaaggtcgaagtgccaaaagtggccactcagaccgtcggcggagtggaactgcccgtggctgctaggcgcagttacctgaatatggaactgacaatccctatttttgccactaattcagattgcgagctgattgtgaaggctatgcaggggctgctgaaagatggaaacccaatcccctcagccattgccgctaatagcggcatctacctcgagccaaccggtgattacaaggatgacgacgataagggtggtggtggtgggtcgaccatgggggaacaacctatcttcagcactcgagctcatgtcttccagatcgacccaaacacaaagaagaactgggtacccaccagcaagcatgcagttactgtgtcttatttctatgacagcacaaggaatgtgtataggataatcagtttagacggctcaaaggcaataataaatagcaccatcactccaaacatgacatttactaaaacatctcaaaagtttggccaatgggctgatagccgggcaaacactgtttatggactgggattctcctctgagcatcatctctcaaaatttgcagaaaagtttcaggaatttaaagaagctgctcggctggcaaaggagaagtcgcaggagaagatggaactgaccagtaccccttcacaggaatcagcaggaggagatcttcagtctcctttaacaccagaaagtatcaatgggacagatgatgagagaacacccgatgtgacacagaactcagagccaagggctgagccagctcagaatgcattgccattttcacatagtgccggggatcgaacccagggcctctctcatgctagttcagccatcagcaaacactgggaggctgaactagccacgctcaaggggaacaatgccaagctcaccgcagcgctgctggagtccactgccaacgtgaagcagtggaagcaacagctggctgcctaccaggaggaggcagagcggctgcacaagcgggtcacggagctggaatgtgttagtagtcaagcaaacgcggtgcacagccacaagacagagctgagtcagacagtgcaggagctggaagagaccctaaaagtaaaggaagaggaaatagaaagattaaaacaagaaattgataacgccagagaacttcaagaacagagggactctttgactcagaaactacaggaagttgagattcgaaataaagacctggaggggcagctgtcggagctggagcagcgcctggagaagagccagagcgagcaggacgctttccgcagtaacctgaagactctcctagagattctggacgggaaaatatttgaactaacagaattgcgggataatttggccaagctactagaatgcagctaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 91 - DNA, 7527 bp, synthetic construct
  source 1..7527: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgacatgaggatcacccatgtcggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagagtcatcctcatcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaaggcgcatggcgttcgtacttaaatatggaactaaccattccaattttctccacgaactccgactgcgagcttattgttaaggcaatgcaaggtctcctaaaagatggaaacccgattccctcagcaatcgcagcaaactccggcatctacggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccaccatgctcgaagtcaaggaagcatcaccaaccagcatccagatcagctgggtgctccacttgcgccacgttcgctactaccgcatcacctacggtgaaactggtggcaatagccctgtccaggaattcaccgtgcctggcagcaagtccactgctaccatcagcggcctgaaacctggtgtcgactataccatcacggtgtacgccgtcacgatcttcagcgcctaccgctccgcctggccgccgatctccatcaactaccgcaccggaaccgattacaaggatgacgacgataagggtagcggctccagtagatctgggctacttaaggccaccatggccagcaacttcacccagtttgtgctggtggacaatggcgggacaggcgatgtgactgtggctccctccaacttcgccaatggggtggctgagtggatcagctccaacagtcggtcacaggcctacaaggtgacctgcagcgtgcggcagtctagtgctcagaagagaaagtacacaattaaggtggaggtgcccaaagtggccacccagacagtgggaggagtggaactgcctgtggctgctcggagatcctacctgaacatggagctgactatccctattttcgccaccaattctgactgtgaactgatcgtgaaggctatgcagggactgctgaaagatggcaaccccatcccttctgccattgccgctaatagtggaatctatggcgcccctgggattcacccagggatgatggcttctaacttcacccaatttgtgctggtcgataatgggggaaccggagatgtgacagtggccccaagtaactttgccaatggcgtggctgaatggatctcaagcaatagccggtcccaggcctacaaagtgacttgctccgtgaggcagtcctctgctcagaagcgcaaatatacaattaaggtcgaagtgccaaaagtggccactcagaccgtcggcggagtggaactgcccgtggctgctaggcgcagttacctgaatatggaactgacaatccctatttttgccactaattcagattgcgagctgattgtgaaggctatgcaggggctgctgaaagatggaaacccaatcccctcagccattgccgctaatagcggcatctacctcgagccagagctctctaggggagctggcgctggagcgggtgcaggggctggctctagattccagtgccggatctgcatgcggaacttcagcgaccggtccaacctgagcaggcacatcagaacccacaccggagaaaagcccttcgcctgcgacatttgcggccggaagttcgccatcagcagcaacctgaacagccacaccaagatccacactggcagccagaaacctttccagtgcagaatttgtatgagaaactttagcagaagcgacaacctggccagacacatccggacacatactggtgaaaaaccttttgcctgtgatatctgtggcagaaagtttgccacctccggcaatctgacccggcacacaaagattcacctgcggggcagccagctatcgattgtcgacgctcctgaacaacgtgaaggtgcttctcaagtttctgtttctgttacttttgaagatgttgctgttctttttactcgtgatgaatggaaaaaacttgatctttctcaacgttctctttatcgtgaagttatgcttgaaaattattctaatcttgcttctatggcttaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 92 - DNA, 6447 bp, synthetic construct
  source 1..6447: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccggggccctgaagaagggccccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagacttccacagagtctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccgccaccatggacgcacaaacacgacgacgtgagcgtcgcgctgagaaacaagctcaatggaaagctgcaaacggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctggatccatgctcgaagtcaaggaagcatcaccaaccagcatccagatcagctggggcaagtacaaggtcatggttcgctactaccgcatcacctacggtgaaactggtggcaatagccctgtccaggaattcaccgtgcctggcagcaagtccactgctaccatcagcagcctgaaacctggtgtcgactataccatcacggtgtacgccgtcacgatcgaccactggaactaccaggacccgatcccgatctccatcaactaccgcaccggatccggcaagcccatccccaaccccctgctgggcctggacagcaccggcggaggtggttctaccggtgacgcacaaacacgacgacgtgagcgtcgcgctgagaaacaagctcaatggaaagctgcaaacgagctctctagatttcagtgccagatttgcatgcgcaactttagccgcaaaagcaccctgaccgatcatattcgcacccataccggcgaaaaaccgtttgcgtgcgatatttgcggccgcaaatttgcggcgcgcagcacccgcaccacccataccaaaattcataccggcagccagaaaccgtttcagtgccgcatttgcatgcgcaactttagccgcagcgatagcctgagcaaacatattcgcacccataccggcgaaaaaccgtttgcgtgcgatatttgcggccgcaaatttgcgcagcgcagcaacctgaaagtgcataccaaaattcatctgcgcggcagccagctgatcgatggtgtcgacgctcctgaacaacgtgaaggtgcttctcaagtttctgtttctgttacttttgaagatgttgctgttctttttactcgtgatgaatggaaaaaacttgatctttctcaacgttctctttatcgtgaagttatgcttgaaaattattctaatcttgcttctatggcttaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 93 - DNA, 6480 bp, synthetic construct
  source 1..6480: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccggggccctgaagaagggccccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggagctggaccaccggaccagcggcgggctccacgcctaccccgggccgcggggcgggcaggtggccaagcccaacgtgatcctgcagatcgggaagtgccgggccgagatgctggagcacgtgcggcggacgcaccggcacctgctggccgaggtgtccaagcaggtggagcgcgagctgaaggggctgcaccggtcggtcgggaagctggagagcaacctggacggctacgtgcccacgagcgactcgcagcgctggaagaagtccatcaaggcctgcctgtgccgctgccaggagaccatcgccaacctggagcgctgggtcaagcgcgagatgcacgtgtggcgcgaggtgttctaccgcctggagcgctgggccgaccgcctggagtccacgggcggcaagtacccggtgggcagcgagtcagcccgccacaccgtttccgtgggcgtggggggtcccgagagctactgccacgaggcagacggctacgactacaccgtcagcccctacgccatcaccccgcccccagccgctggcgagctgcccgggcaggagcccgccgaggcccagcagtaccagccgtgggtccccggcgaggacgggcagcccagccccggcgtggacacgcagatcttcgaggaccctcgagagttcctgagccacctagaggagtacttgcggcaggtgggcggctctgaggagtactggctgtcccagatccagaatcacatgaacgggccggccaagaagtggtgggagttcaagcagggctccgtgaagaactgggtggagttcaagaaggagttcctgcagtacagcgagggcacgctgtcccgagaggccatccagcgcgagctggacctgccgcagaagcagggcgagccgctggaccagttcctgtggcgcaagcgggacctgtaccagacgctctacgtggacgcggacgaggaggagatcatccagtacgtggtgggcaccctgcagcccaagctcaagcgtttcctgcgccaccccctgcccaagaccctggagcagctcatccagaggggcatggaggtgcaggatgacctggagcaggcggccgagccggccggcccccacctcccggtggaggatgaggcggagaccctcacgcccgcccccaacagcgagtccgtggccagtgaccggacccagcccgagggctcaggaggatctggtggttctgattacaaggatgacgacgataagggcggaggtggttctgacgcacaaacacgacgacgtgagcgtcgcgctgagaaacaagctcaatggaaagctgcaaactaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 94 - DNA, 5864 bp, synthetic construct
  source 1..5864: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgccaccatggggtcttcaaaatctaaaccaaaggaccccagccagcgcggcggaggtggttctgacgcacaaacacgacgacgtgagcgtcgcgctgagaaacaagctcaatggaaagctgcaaacggcggaggtggttctgattacaaggatgacgacgataagtaagaattctgcagatatcgctcgctttcttgctgtccaatttctattaaaggttcctttgttccctaagctcgaccaaaggttcctttgtggccctgaaaaagggccaaattggtggctggtgtggctaatgccctatggccctgaaaaagggccactggaggatattcatgcactcgaccaaaggttcctttgtggccctgaaaaagggccaaattggtggctggtgtggctaatgccctatggccctgaaaaagggccactggaggatattcatgcactcgagtactaaactgggggatattatgaagggccttgagcatctggattctgcctaattctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 95 - DNA, 5912 bp, synthetic construct
  source 1..5912: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgccaccatgctgtgctgcatcagaagaactaaaccggttgagaagaatgaagaggccgatcaggagctgcagtcgacggtgccgcgggcccgggatccaccggtcgccaccgacgcacaaacacgacgacgtgagcgtcgcgctgagaaacaagctcaatggaaagctgcaaacggcggaggtggttctgattacaaggatgacgacgataagtaagaattctgcagatatcgctcgctttcttgctgtccaatttctattaaaggttcctttgttccctaagctcgaccaaaggttcctttgtggccctgaaaaagggccaaattggtggctggtgtggctaatgccctatggccctgaaaaagggccactggaggatattcatgcactcgaccaaaggttcctttgtggccctgaaaaagggccaaattggtggctggtgtggctaatgccctatggccctgaaaaagggccactggaggatattcatgcactcgagtactaaactgggggatattatgaagggccttgagcatctggattctgcctaattctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 96 - DNA, 5882 bp, synthetic construct
  source 1..5882: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgccaccatggattacaaggatgacgacgataagggcggaggtggttctgacgcacaaacacgacgacgtgagcgtcgcgctgagaaacaagctcaatggaaagctgcaaacggcggaggtggttctaagctgaaccctcctgatgagagtggccccggctgcatgagctgctgtgtgctctcctaagaattctgcagatatcgctcgctttcttgctgtccaatttctattaaaggttcctttgttccctaagctcgaccaaaggttcctttgtggccctgaaaaagggccaaattggtggctggtgtggctaatgccctatggccctgaaaaagggccactggaggatattcatgcactcgaccaaaggttcctttgtggccctgaaaaagggccaaattggtggctggtgtggctaatgccctatggccctgaaaaagggccactggaggatattcatgcactcgagtactaaactgggggatattatgaagggccttgagcatctggattctgcctaattctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 97 - DNA, 6082 bp, synthetic construct
  source 1..6082: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattacaaggatgacgacgataagggcggaggtggttctgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaaggcgcatggcgttcgtacttaaatatggaactaaccattccaattttctccacgaactccgactgcgagcttattgttaaggcaatgcaaggtctcctaaaagatggaaacccgattccctcagcaatcgcagcaaactccggcatctacggcggaggtggttctaagctgaaccctcctgatgagagtggccccggctgcatgagctgctgtgtgctctcctaagaattctgcagatatccctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 98 - DNA, 6143 bp, synthetic construct
  source 1..6143: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattctgcagatatcgatcctaaggtacctaattgcctagaaaggagcagacgatatggcgtcgctccctgcaggtcgactctagaaaccagcagagcatatgggctcgctggctgcagtattcccgggttcattagatcctaaggtacctaattgcctagaaaggagcagacgatatggcgtcgctccctgcaggtcgactctagaaaccagcagagcatatgggctcgctggctgcagtattcccgggttcattagatctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 99 - DNA, 4719 bp, synthetic construct
  source 1..4719: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccgccaccatggattacaaggatgacgacgataagggcggaggtggttctgacgcacaaacacgacgacgtgagcgtcgcgctgagaaacaagctcaatggaaagctgcaaacggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 100 - DNA, 5010 bp, synthetic construct
  source 1..5010: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggattacaaggatgacgacgataagggcggaggtggttctgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaaggcgcatggcgttcgtacttaaatatggaactaaccattccaattttctccacgaactccgactgcgagcttattgttaaggcaatgcaaggtctcctaaaagatggaaacccgattccctcagcaatcgcagcaaactccggcatctacggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 101 - DNA, 5043 bp, synthetic construct
  source 1..5043: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 102 - DNA, 5337 bp, synthetic construct
  source 1..5337: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccggggccctgaagaagggccccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccgccaccatggattacaaggatgacgacgataagggcggaggtggttctgacgcacaaacacgacgacgtgagcgtcgcgctgagaaacaagctcaatggaaagctgcaaacggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 103 - DNA, 5628 bp, synthetic construct
  source 1..5628: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgacatgaggatcacccatgtcggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggattacaaggatgacgacgataagggcggaggtggttctgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaaggcgcatggcgttcgtacttaaatatggaactaaccattccaattttctccacgaactccgactgcgagcttattgttaaggcaatgcaaggtctcctaaaagatggaaacccgattccctcagcaatcgcagcaaactccggcatctacggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 104 - DNA, 5667 bp, synthetic construct
  source 1..5667: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 105 - DNA, 5535 bp, synthetic construct
  source 1..5535: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggcactcttccgtggtctggtggataaattcgttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaacgacgtcagaccacgggggagtgcccttttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 106 - DNA, 5725 bp, synthetic construct
  source 1..5725: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatcctaggttaaggatgcaccgacgggacgttctatgaggacgaatctcccgcttataagattctataaactgtgcggtccttcaattggggtccccactaacctaagacaggagggccgggaaacctgcctaatccaatgacgggtaatagtggggacccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 107 - DNA, 5820 bp, synthetic construct
  source 1..5820: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 108 - DNA, 7092 bp, synthetic construct
  source 1..7092: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatgggcaagcccatccccaaccccctgctgggcctggacagcaccggcagcggcaactatgagcttttgaccactgagaacgctcctgttaagatgtggacaaaaggcgtgcctgtagaggccgacgctcggcagcaactcattaacaccgccaagatgccctttattttcaagcatattgccgtgatgcctgatgtccatcttggtaagggttcaacaatcgggagcgtcatccctaccaagggtgccatcattccagccgccgtaggagtagatattggatgcggcatgaacgcacttagaacagctctgaccgccgaggatcttcccgagaacctcgctgaactgcgacaggcaatcgagacagcagttcctcacggcagaaccacaggcaggtgtaagagagataagggcgcatgggaaaacccccccgtgaatgtcgacgcaaaatgggcagagttggaagctgggtatcaatggctgacccaaaagtacccacggttcctcaatactaataactataagcaccttgggacactcggaaccggcaaccacttcatagaaatatgcctggacgagtcagatcaagtttggataatgctccactctggttcacggggcattggcaacgctataggaacatactttatagacctggcccagaaagagatgcaagaaacattggaaactctcccaagtagggacctcgcttacttcatggagggaactgagtatttcgatgattatctgaaagccgtagcatgggcacagttgttcgcctccttgaatagggatgcaatgatggagaatgtcgtcactgctcttcaaagtatcacccaaaaaacagtacgccaacctcagactctggcaatggaagagatcaactgtcatcataactacgtacaaaaggaacaacacttcggcgaagagatctatgttacccggaaaggggccgtctcagctagggcaggccaatacggcataatccctggctctatgggtgcaaaaagcttcatagttcgaggccttgggaacgaggagagcttttgtagctgtagccacggggctggtcgggtgatgtcccggactaaagctaaaaaattgttctctgttgaggaccaaatacgggctaccgcacacgtagaatgccggaaggacgccgaggtcatcgacgaaatccctatggcctacaaggacattgacgcagttatggccgcacagtctgacctggtggaagttatatatacactgaggcaagtagtatgtgtgaagggatctggtggttctcccaagaagaagaggaaggtggaccccaagaagaagaggaaggtggaccccaagaagaagaggaaggtgggctcaggaggagagggcagaggaagtcttctaacatgcggtgacgtggaggagaatcccggccctgccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 109 - DNA, 7068 bp, synthetic construct
  source 1..7068: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatgggcaagcccatccccaaccccctgctgggcctggacagcaccggcagcggcgcagaacaggatgtggaaaacgatcttttggattacgatgaagaggaagagccccaggctcctcaagagagcacaccagctccccctaagaaagacatcaagggatcctacgtttccatccacagctctggcttccgggactttctgctgaagccggagctcctgcgggccatcgtggactgtggctttgagcatccttctgaggtccagcatgagtgcattccccaggccatcctgggcatggacgtcctgtgccaggccaagtccgggatgggcaagacagcggtcttcgtgctggccaccctacagcagattgagcctgtcaacggacaggtgacggtcctggtcatgtgccacacgagggagctggccttccagatcagcaaggaatatgagcgcttttccaagtacatgcccagcgtcaaggtgtctgtgttcttcggtggtctctccatcaagaaggatgaagaagtgttgaagaagaactgtccccatgtcgtggtggggaccccgggccgcatcctggcgctcgtgcggaataggagcttcagcctaaagaatgtgaagcactttgtgctggacgagtgtgacaagatgctggagcagctggacatgcggcgggatgtgcaggagatcttccgcctgacaccacacgagaagcagtgcatgatgttcagcgccaccctgagcaaggacatccggcctgtgtgcaggaagttcatgcaggatccaatggaggtgtttgtggacgacgagaccaagctcacgctgcacggcctgcagcagtactacgtcaaactcaaagacagtgagaagaaccgcaagctctttgatctcttggatgtgctggagtttaaccaggtgataatcttcgtcaagtcagtgcagcgctgcatggccctggcccagctcctcgtggagcagaacttcccggccatcgccatccaccggggcatggcccaggaggagcgcctgtcacgctatcagcagttcaaggatttccagcggcggatcctggtggccaccaatctgtttggccgggggatggacatcgagcgagtcaacatcgtctttaactacgacatgcctgaggactcggacacctacctgcaccgggtggcccgggcgggtcgctttggcaccaaaggcctagccatcacttttgtgtctgacgagaatgatgccaaaatcctcaatgacgtccaggaccggtttgaagttaatgtggcagaacttccagaggaaatcgacatctccacatacatcgagcagagccggtctggtggttctgagggcagaggaagtcttctaacatgcggtgacgtggaggagaatcccggccctgccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 110 - DNA, 6076 bp, synthetic construct
  source 1..6076: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgcttaagaaaaaaaaaggccgggcgcggtggctcacgcctgtaatcccagctctcagggaggctaagaggcgggaggatagcttgagcccaggagttcgagacctgcctgggcaatatagcgagaccccgttctccagaaaaaggaaaaaaaaaaacaaaagacaaaaaaaaaataagcgtaacttccctcaaagcaacaacccccccccccctttaaaaaaaaagttaaccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 111 - DNA, 6396 bp, synthetic construct
  source 1..6396: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 112 - DNA, 6191 bp, synthetic construct
  source 1..6191: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctaatgattaacccgccatgctacttatctacgtagccatgctctaggaagatcgtaccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 113 - DNA, 6920 bp, synthetic construct
  source 1..6920: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattggggtcccatcattcatggcaagtggccgcggtcggcgtggactgtagaacactgccaatgccggtcccaagcccggataaaagtggagggtacagtccacgctttttttctagactgcagagggccctaatgattaacccgccatgctacttatctacgtagccatgctctaggaagatcgtaccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 114 - DNA, 6965 bp, synthetic construct
  source 1..6965: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatcctagggacgggacgttctatgaggacgaatctcccgcttataagattctataaactgtgcggtccttcaattggggtccccactaacctaagacaggagggccgggaaacctgcctaatccaatgacgggtaatagtggggacccatcattcatggcaagtggccgcggtcggcgtggactgtagaacactgccaatgccggtcccaagcccggataaaagtggagggtacagtccacgctttttttctagactgcagagggccctaatgattaacccgccatgctacttatctacgtagccatgctctaggaagatcgtaccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 115 - DNA, 6426 bp, synthetic construct
  source 1..6426: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctaggttaaggatgcaccgacgggacgttctatgaggacgaatctcccgcttataagattctataaactgtgcggtccttcaattgaaaaaaaaaaaaaaaaaaaaaaaaaaaaaagggtcccatcattcatggcaagtggccgcggtcggcgtggactgtagaacactgccaatgccggtcccaagcccggataaaagtggagggtacagtccacgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccatggattacaaggatgacgacgataagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 116 - DNA, 7030 bp, synthetic construct
  source 1..7030: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccgggagcagacgatatggcgtcgctcccggtccgatactctgatgatgggtccccctagggacgggacgttctgctaagatcatttctccctggggcagattctataaactgtgcggtccttcaattgaaaaaaaaaaaaaaaaaaaaaaaaaaaaaagggtcccatcattcatggcaagtggccgcggtcggcgtggactgtagaacactgccaatgccggtcccaagcccggataaaagtggagggtacagtccacgctttttttctagactgcagagggccctcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaagctaccccctatataagcagagctcgtttagtgaaccgtcagatcgcctggagacgccatccacgctgttttgacctccatagaagacaccgggaccgatccagcctggatccgccaccatgggcaagcccatccccaaccccctgctgggcctggacagcaccggcggtggaggttcctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggtggcggatctggcggcggtggtagcaatgattttggcaattacaacaatcagtcttccaattttgggccgatgaagggaggaaactttggaggcaggagctctggcccttatggtggtggaggccagtactttgctaaaccacggaaccaaggtggctatggcggaggtggttctctgcctccacttgaaagactgacactgggctcaggaggatctggtggttctgagggcagaggaagtcttctaacatgcggtgacgtggaggagaatcccggccctgccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcggaggtggttcttccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtggcggaggtggttctaagctgaaccctcctgatgagagtggccccggctgcatgagctgctgtgtgctctcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 117 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 118 - DNA, 42 bp, synthetic construct
  source 1..42: mol_type = other DNA
ggcaagcccatccccaaccccctgctgggcctggacagcacc

SEQ ID NO: 119 - DNA, 19 bp, synthetic construct
  source 1..19: mol_type = other DNA
tccctatcagtgatagaga

SEQ ID NO: 120 - DNA, 54 bp, synthetic construct
  source 1..54: mol_type = other DNA
gagggcagaggaagtcttctaacatgcggtgacgtggaggagaatcccggccct

SEQ ID NO: 121 - DNA, 330 bp, synthetic construct
  source 1..330: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 122 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 123 - DNA, 136 bp, synthetic construct
  source 1..136: mol_type = other DNA
atcccgcccctaactccgcccagttccgcccattctccgccccatggctgactaattttttttatttatgcagaggccgaggccgcctctgcctctgagctattccagaagtagtgaggaggcttttttggaggcc

SEQ ID NO: 124 - DNA, 21 bp, synthetic construct
  source 1..21: mol_type = other DNA
cccaagaagaagaggaaggtg

SEQ ID NO: 125 - DNA, 918 bp, synthetic construct
  source 1..918: mol_type = other DNA
gacgtggtgaatcagctggtggctgggggtcagttccgggtggtcaaggagccccttggcttcgtgaaggtgctgcagtgggtctttgccatcttcgcctttgctacgtgtggcagctacaccggggagcttcggctgagcgtggagtgtgccaacaagacggagagtgccctcaacatcgaagttgaattcgagtaccccttcaggctgcaccaagtgtactttgatgcaccctcctgcgtcaaagggggcactaccaagatcttcctggttggggactactcctcgtcggctgaattctttgtcaccgtggctgtgtttgccttcctctactccatgggggccctggccacctacatcttcctgcagaacaagtaccgagagaacaacaaagggcctatgatggactttctggctacagccgtgttcgctttcatgtggctagttagttcatcagcctgggccaaaggcctgtccgatgtgaagatggccacggacccagagaacattatcaaggagatgcccatgtgccgccagacagggaacacatgcaaggaactgagggaccctgtgacttcaggactcaacacctcagtggtgtttggcttcctgaacctggtgctctgggttggcaacttatggttcgtgttcaaggagacaggctgggcagccccattcatgcgcgcacctccaggcgccccggaaaagcaaccagcacctggcgatgcctacggcgatgcgggctacgggcagggccccggaggctatgggccccaggactcctacgggcctcagggtggttatcaacccgattacgggcagccagccagcggtggcggtggctacgggcctcagggcgactatgggcagcaaggctatggccaacagggtgcgcccacctccttctccaatcagatg

SEQ ID NO: 126 - DNA, 384 bp, synthetic construct
  source 1..384: mol_type = other DNA
atgtccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgt

SEQ ID NO: 127 - DNA, 589 bp, synthetic construct
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 128 - DNA, 586 bp, synthetic construct
  source 1..586: mol_type = other DNA
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaa

SEQ ID NO: 129 - DNA, 105 bp, synthetic construct
  source 1..105: mol_type = other DNA
atgctgtgctgcatcagaagaactaaaccggttgagaagaatgaagaggccgatcaggagctgcagtcgacggtgccgcgggcccgggatccaccggtcgccacc

SEQ ID NO: 130 - DNA, 27 bp, synthetic construct
  source 1..27: mol_type = other DNA
ctgcctccacttgaaagactgacactg

SEQ ID NO: 131 - DNA, 795 bp, synthetic construct
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 132 - DNA, 42 bp, synthetic construct
  source 1..42: mol_type = other DNA
atggggtcttcaaaatctaaaccaaaggaccccagccagcgc

SEQ ID NO: 133 - DNA, 32 bp, synthetic construct
  source 1..32: mol_type = other DNA
ggcactcttccgtggtctggtggataaattcg

SEQ ID NO: 134 - DNA, 26 bp, synthetic construct
  source 1..26: mol_type = other DNA
cgacgtcagaccacgggggagtgccc

SEQ ID NO: 135 - DNA, 390 bp, synthetic construct
  source 1..390: mol_type = other DNA
atggccagcaacttcacccagtttgtgctggtggacaatggcgggacaggcgatgtgactgtggctccctccaacttcgccaatggggtggctgagtggatcagctccaacagtcggtcacaggcctacaaggtgacctgcagcgtgcggcagtctagtgctcagaagagaaagtacacaattaaggtggaggtgcccaaagtggccacccagacagtgggaggagtggaactgcctgtggctgctcggagatcctacctgaacatggagctgactatccctattttcgccaccaattctgactgtgaactgatcgtgaaggctatgcagggactgctgaaagatggcaaccccatcccttctgccattgccgctaatagtggaatctat

SEQ ID NO: 136 - DNA, 708 bp, synthetic construct
  source 1..708: mol_type = other DNA
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 137 - DNA, 167 bp, synthetic construct
  source 1..167: mol_type = other DNA
gctcctgaacaacgtgaaggtgcttctcaagtttctgtttctgttacttttgaagatgttgctgttctttttactcgtgatgaatggaaaaaacttgatctttctcaacgttctctttatcgtgaagttatgcttgaaaattattctaatcttgcttctatggctta

SEQ ID NO: 138 - DNA, 168 bp, synthetic construct
  source 1..168: mol_type = other DNA
gctcctgaacaacgtgaaggtgcttctcaagtttctgtttctgttacttttgaagatgttgctgttctttttactcgtgatgaatggaaaaaacttgatctttctcaacgttctctttatcgtgaagttatgcttgaaaattattctaatcttgcttctatggcttaa

SEQ ID NO: 139 - DNA, 19 bp, synthetic construct
  source 1..19: mol_type = other DNA
taatacgactcactatagg

SEQ ID NO: 140 - DNA, 10 bp, synthetic construct
  source 1..10: mol_type = other DNA
gccaccatgg

SEQ ID NO: 141 - RNA, 154 nt, synthetic construct
  source 1..154: mol_type = other RNA
ggggttggggatttagctcagtggtagagcgcttgcctagcaagcgcaaggccctgggttcggtcctcagctctggaaaaaaaaaaaaaaaaaaaaaaagacaaaataacaaaaagaccaaaaaaaaacaaggtaactggcacacacaaccttt

SEQ ID NO: 142 - DNA, 345 bp, synthetic construct
  source 1..345: mol_type = other DNA
tgccagatttgcatgcgcaactttagccgcaaaagcaccctgaccgatcatattcgcacccataccggcgaaaaaccgtttgcgtgcgatatttgcggccgcaaatttgcggcgcgcagcacccgcaccacccataccaaaattcataccggcagccagaaaccgtttcagtgccgcatttgcatgcgcaactttagccgcagcgatagcctgagcaaacatattcgcacccataccggcgaaaaaccgtttgcgtgcgatatttgcggccgcaaatttgcgcagcgcagcaacctgaaagtgcataccaaaattcatctgcgcggcagccagctgatcgatggt

SEQ ID NO: 143 - DNA, 1185 bp, synthetic construct
  source 1..1185: mol_type = other DNA
gagctggaccaccggaccagcggcgggctccacgcctaccccgggccgcggggcgggcaggtggccaagcccaacgtgatcctgcagatcgggaagtgccgggccgagatgctggagcacgtgcggcggacgcaccggcacctgctggccgaggtgtccaagcaggtggagcgcgagctgaaggggctgcaccggtcggtcgggaagctggagagcaacctggacggctacgtgcccacgagcgactcgcagcgctggaagaagtccatcaaggcctgcctgtgccgctgccaggagaccatcgccaacctggagcgctgggtcaagcgcgagatgcacgtgtggcgcgaggtgttctaccgcctggagcgctgggccgaccgcctggagtccacgggcggcaagtacccggtgggcagcgagtcagcccgccacaccgtttccgtgggcgtggggggtcccgagagctactgccacgaggcagacggctacgactacaccgtcagcccctacgccatcaccccgcccccagccgctggcgagctgcccgggcaggagcccgccgaggcccagcagtaccagccgtgggtccccggcgaggacgggcagcccagccccggcgtggacacgcagatcttcgaggaccctcgagagttcctgagccacctagaggagtacttgcggcaggtgggcggctctgaggagtactggctgtcccagatccagaatcacatgaacgggccggccaagaagtggtgggagttcaagcagggctccgtgaagaactgggtggagttcaagaaggagttcctgcagtacagcgagggcacgctgtcccgagaggccatccagcgcgagctggacctgccgcagaagcagggcgagccgctggaccagttcctgtggcgcaagcgggacctgtaccagacgctctacgtggacgcggacgaggaggagatcatccagtacgtggtgggcaccctgcagcccaagctcaagcgtttcctgcgccaccccctgcccaagaccctggagcagctcatccagaggggcatggaggtgcaggatgacctggagcaggcggccgagccggccggcccccacctcccggtggaggatgaggcggagaccctcacgcccgcccccaacagcgagtccgtggccagtgaccggacccagcccgag

SEQ ID NO: 144 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 145 - DNA, 27 bp, synthetic construct
  source 1..27: mol_type = other DNA
tatccatatgatgttccagattatgct

SEQ ID NO: 146 - DNA, 24 bp, synthetic construct
  source 1..24: mol_type = other DNA
gattacaaggatgacgacgataag

SEQ ID NO: 147 - DNA, 12 bp, synthetic construct
  source 1..12: mol_type = other DNA
tcggccctcaat

SEQ ID NO: 148 - DNA, 21 bp, synthetic construct
  source 1..21: mol_type = other DNA
gggtcccatcattcatggcaa

SEQ ID NO: 149 - DNA, 20 bp, synthetic construct
  source 1..20: mol_type = other DNA
cggtccgatactctgatgat

SEQ ID NO: 150 - DNA, 20 bp, synthetic construct
  source 1..20: mol_type = other DNA
ttgccatgtgtatcggtccg

SEQ ID NO: 151 - DNA, 429 bp, synthetic construct
  source 1..429: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatt

SEQ ID NO: 152 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 153 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 154 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 155 - DNA, 60 bp, synthetic construct
  source 1..60: mol_type = other DNA
aagctgaaccctcctgatgagagtggccccggctgcatgagctgctgtgtgctctcctaa

SEQ ID NO: 156 - DNA, 342 bp, synthetic construct
  source 1..342: mol_type = other DNA
tgccggatctgcatgcggaacttcagcgaccggtccaacctgagcaggcacatcagaacccacaccggagaaaagcccttcgcctgcgacatttgcggccggaagttcgccatcagcagcaacctgaacagccacaccaagatccacactggcagccagaaacctttccagtgcagaatttgtatgagaaactttagcagaagcgacaacctggccagacacatccggacacatactggtgaaaaaccttttgcctgtgatatctgtggcagaaagtttgccacctccggcaatctgacccggcacacaaagattcacctgcggggcagccagctatcgatt

SEQ ID NO: 157 - DNA, 993 bp, synthetic construct
  source 1..993: mol_type = other DNA
taatgattaacccgccatgctacttatctacgtagccatgctctaggaagatcgtaccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 158 - DNA, 19 bp, synthetic construct
  source 1..19: mol_type = other DNA
gggccctgaagaagggccc

SEQ ID NO: 159 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 160 - DNA, 25 bp, synthetic construct
  source 1..25: mol_type = other DNA
tatgaggacgaatctcccgcttata

SEQ ID NO: 161 - DNA, 87 bp, synthetic construct
  source 1..87: mol_type = other DNA
cctaggttaaggatgcaccgacgggacgttctatgaggacgaatctcccgcttataagattctataaactgtgcggtccttcaattg

SEQ ID NO: 162 - DNA, 16 bp, synthetic construct
  source 1..16: mol_type = other DNA
gaattctgcagatatc

SEQ ID NO: 163 - DNA, 105 bp, synthetic construct
  source 1..105: mol_type = other DNA
actcttcctttttcaatattattgaagcatttatcagggttattgtctcatgagcggatacatatttgaatgtatttagaaaaataaacaaataggggttccgcg

SEQ ID NO: 164 - DNA, 861 bp, synthetic construct
  source 1..861: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 165 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 166 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 167 - DNA, 11 bp, synthetic construct
  source 1..11: mol_type = other DNA
gcggacgagga

SEQ ID NO: 168 - DNA, 55 bp, synthetic construct
  source 1..55: mol_type = other DNA
gccgcactcgccggtcccaagcccggataaaatgggagggggcgggaaaccgcct

SEQ ID NO: 169 - DNA, 21 bp, synthetic construct
  source 1..21: mol_type = other DNA
aaccatgccgagtgcggccgc

SEQ ID NO: 170 - DNA, 25 bp, synthetic construct
  source 1..25: mol_type = other DNA
ttaaggatgcaccgacgggacgttc

SEQ ID NO: 171 - DNA, 26 bp, synthetic construct
  source 1..26: mol_type = other DNA
gtggccgcggtcggcgtggactgtag

SEQ ID NO: 172 - DNA, 55 bp, synthetic construct
  source 1..55: mol_type = other DNA
aacactgccaatgccggtcccaagcccggataaaagtggagggtacagtccacgc

SEQ ID NO: 173 - DNA, 25 bp, synthetic construct
  source 1..25: mol_type = other DNA
agattctataaactgtgcggtcctt

SEQ ID NO: 174 - protein, 286 aa, synthetic construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 175 - protein, 114 aa, synthetic construct
CRICMRNFSDRSNLSRHIRTHTGEKPFACDICGRKFAISSNLNSHTKIHTGSQKPFQCRICMRNFSRSDNLARHIRTHTGEKPFACDICGRKFATSGNLTRHTKIHLRGSQLSI

SEQ ID NO: 176 - protein, 407 aa, synthetic construct
NYELLTTENAPVKMWTKGVPVEADARQQLINTAKMPFIFKHIAVMPDVHLGKGSTIGSVIPTKGAIIPAAVGVDIGCGMNALRTALTAEDLPENLAELRQAIETAVPHGRTTGRCKRDKGAWENPPVNVDAKWAELEAGYQWLTQKYPRFLNTNNYKHLGTLGTGNHFIEICLDESDQVWIMLHSGSRGIGNAIGTYFIDLAQKEMQETLETLPSRDLAYFMEGTEYFDDYLKAVAWAQLFASLNRDAMMENVVTALQSITQKTVRQPQTLAMEEINCHHNYVQKEQHFGEEIYVTRKGAVSARAGQYGIIPGSMGAKSFIVRGLGNEESFCSCSHGAGRVMSRTKAKKLFSVEDQIRATAHVECRKDAEVIDEIPMAYKDIDAVMAAQSDLVEVIYTLRQVVCVKG

SEQ ID NO: 177 - protein, 4 aa, synthetic construct
IEGR

SEQ ID NO: 178 - protein, 8 aa, synthetic construct
DYKDDDDK

SEQ ID NO: 179 - protein, 9 aa, synthetic construct
YPYDVPDYA

SEQ ID NO: 180 - protein, 395 aa, synthetic construct
ELDHRTSGGLHAYPGPRGGQVAKPNVILQIGKCRAEMLEHVRRTHRHLLAEVSKQVERELKGLHRSVGKLESNLDGYVPTSDSQRWKKSIKACLCRCQETIANLERWVKREMHVWREVFYRLERWADRLESTGGKYPVGSESARHTVSVGVGGPESYCHEADGYDYTVSPYAITPPPAAGELPGQEPAEAQQYQPWVPGEDGQPSPGVDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDADEEEIIQYVVGTLQPKLKRFLRHPLPKTLEQLIQRGMEVQDDLEQAAEPAGPHLPVEDEAETLTPAPNSESVASDRTQPE

SEQ ID NO: 181 - protein, 115 aa, synthetic construct
CQICMRNFSRKSTLTDHIRTHTGEKPFACDICGRKFAARSTRTTHTKIHTGSQKPFQCRICMRNFSRSDSLSKHIRTHTGEKPFACDICGRKFAQRSNLKVHTKIHLRGSQLIDG

SEQ ID NO: 182 - protein, 55 aa, synthetic construct
APEQREGASQVSVSVTFEDVAVLFTRDEWKKLDLSQRSLYREVMLENYSNLASMA

SEQ ID NO: 183 - protein, 235 aa, synthetic construct; identical to SEQ ID NO: 12

SEQ ID NO: 184 - protein, 130 aa, synthetic construct
MASNFTQFVLVDNGGTGDVTVAPSNFANGVAEWISSNSRSQAYKVTCSVRQSSAQKRKYTIKVEVPKVATQTVGGVELPVAARRSYLNMELTIPIFATNSDCELIVKAMQGLLKDGNPIPSAIAANSGIY

SEQ ID NO: 185 - protein, 14 aa, synthetic construct
MGSSKSKPKDPSQR

SEQ ID NO: 186 - protein, 264 aa, synthetic construct [sequence omitted]

SEQ ID NO: 187 - protein, 9 aa, synthetic construct
LPPLERLTL

SEQ ID NO: 188 - protein, 35 aa, synthetic construct
MLCCIRRTKPVEKNEEADQELQSTVPRARDPPVAT

SEQ ID NO: 189 - protein, 121 aa, synthetic construct
SVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 190 - protein, 127 aa, synthetic construct
SKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 191 - protein, 366 aa, synthetic construct; identical to SEQ ID NO: 28

SEQ ID NO: 192 - protein, 306 aa, synthetic construct
DVVNQLVAGGQFRVVKEPLGFVKVLQWVFAIFAFATCGSYTGELRLSVECANKTESALNIEVEFEYPFRLHQVYFDAPSCVKGGTTKIFLVGDYSSSAEFFVTVAVFAFLYSMGALATYIFLQNKYRENNKGPMMDFLATAVFAFMWLVSSSAWAKGLSDVKMATDPENIIKEMPMCRQTGNTCKELRDPVTSGLNTSVVFGFLNLVLWVGNLWFVFKETGWAAPFMRAPPGAPEKQPAPGDAYGDAGYGQGPGGYGPQDSYGPQGGYQPDYGQPASGGGGYGPQGDYGQQGYGQQGAPTSFSNQM

SEQ ID NO: 193 - protein, 7 aa, synthetic construct
PKKKRKV

SEQ ID NO: 194 - protein, 18 aa, synthetic construct
EGRGSLLTCGDVEENPGP

SEQ ID NO: 195 - protein, 14 aa, synthetic construct
GKPIPNPLLGLDST

SEQ ID NO: 196 - RNA, 234 nt, synthetic construct
  source 1..234: mol_type = other RNA
aggagctatgttccttgggttcttgggagcagcaggaagcactatgggcgcagtgtcaatgacgctgacggtacaggccagacaattattgtctggtatagtgcaacagcagaacaatttgctgagggctattgaggcgcaacaacatctgttgcaactcacagtctggggcatcaagcagctccaggcaagagtcctggctgtggaaagatacctaagggatacacagctcct

SEQ ID NO: 197 - RNA, 142 nt, synthetic construct
  source 1..142: mol_type = other RNA
agactggacagccaatgacgggtaagagagtgacatttctcactaacctaagacaggagggccgtcaaagctactgcctaatccaatgacgggtaatagtgacaagaaatgtatcactccaacctaagacaggcgcagcctc

SEQ ID NO: 198 - RNA, 62 nt, synthetic construct
  source 1..62: mol_type = other RNA
ggcactaacctaagacaggagggccgggaaacctgcctaatccaatgacgggtaatagtgtc

(19) United States
(12) Patent Application Publication
(10) Pub. No.: US 2025/0092397 A1
(43) Pub. Date: Mar. 20, 2025

(54) RIBOZYME-ASSISTED CIRCULAR RNAS AND COMPOSITIONS AND METHODS OF USE THEREOF

(71) Applicants: The Broad Institute, Inc., Cambridge, MA (US); Massachusetts Institute of Technology, Cambridge, MA (US)

(72) Inventors: Hailing Shi, Cambridge, MA (US); Yiming Zhou, Cambridge, MA (US); Xiao Wang, Cambridge, MA (US)

(73) Assignees: The Broad Institute, Inc., Cambridge, MA (US); Massachusetts Institute of Technology, Cambridge, MA (US)

(21) Appl. No.: 18/956,207

(22) Filed: Nov. 22, 2024

Related U.S. Application Data
(63) Continuation of application No. PCT/US2023/023674, filed on May 26, 2023.
(60) Provisional application No. 63/385,553, filed on Nov. 30, 2022.
(60) Provisional application No. 63/346,729, filed on May 27, 2022.

(51) Int. Cl. C12N 15/113; C12N 15/66; C12N 15/86; C12Q 1/6841; C12Q 1/6881; C12Q 1/70; G16B 25/10; G16B 50/30

(52) U.S. Cl. CPC C12N 15/113; C12N 15/66; C12N 15/86; C12Q 1/6841; C12Q 1/6881; C12Q 1/70; G16B 25/10; G16B 50/30; C12N 2310/122; C12N 2750/14143

(57) ABSTRACT

The disclosure features compositions, systems, and methods for preparation and use of efficient RNA nuclear export of ribozyme-assisted circular RNA molecules (racRNAs). In embodiments, the methods involve characterizing a cell or tissue using racRNAs.

### CROSS-REFERENCE TO RELATED APPLICATIONS

This application is a continuation under 35 U.S.C. § 111(a) of PCT International Patent Application No. PCT/US2023/023674, filed May 26, 2023, designating the United States and published in English, which claims priority to and the benefit of U.S. Provisional Application No. 63/346,729, filed May 27, 2022, and U.S. Provisional Application No. 63/385,553, filed Nov. 30, 2022, the entire contents of which are incorporated herein by reference.

### SEQUENCE LISTING

This application contains a Sequence Listing which has been submitted electronically in XML format and is hereby incorporated by reference in its entirety. The Sequence Listing XML file, created on Jun. 23, 2023, is named “167741-049202_PCT_SL.xml” and is 433,285 bytes in size.

### BACKGROUND OF THE INVENTION

Advances in next-generation sequencing technologies have led to discoveries and characterization of expanding categories of RNA species, such as short and long non-coding RNAs, circular RNAs, extracellular vesicle RNAs, guide RNAs, etc. They not only add to the rich knowledge of RNA biology but can also be flexibly engineered as vessels for various functional tools, including genetic circuits and biosensing. For live-cell application and therapeutic purposes, RNA expression systems can be delivered into cells in the form of purified RNA, plasmids, or viral genomes. However, the efficacy of synthetic RNAs depends on the efficient localization of the functional RNA species towards specific cellular compartments of interest.

Elements capable of directing the localization of synthetic RNAs at the subcellular level are desired.

### SUMMARY OF THE INVENTION

As described below, the present invention features compositions, systems, and methods for the preparation and use of elements that mediate RNA nuclear export and subcellular localization of ribozyme-assisted circular RNA molecules (racRNAs). In embodiments, the methods involve characterizing a cell or tissue using racRNAs.

In one aspect, the disclosure features an RNA polynucleotide containing the following elements, each of which is operably linked: i) a first ribozyme; ii) a first ligation sequence; iii) an RNA hairpin sequence; iv) a heterologous polynucleotide; v) a second ligation sequence; and vi) a second ribozyme. The RNA hairpin sequence specifically binds an RNA binding polypeptide that mediates nuclear export.

In another aspect, the disclosure features an expression vector encoding the RNA polynucleotide of any aspect provided herein, or embodiments thereof.

In another aspect, the disclosure features a circular RNA polynucleotide containing an RNA hairpin sequence and a heterologous polynucleotide, where the RNA hairpin sequence specifically binds an RNA binding protein that mediates nuclear export.

In another aspect, the disclosure features a cell containing the RNA polynucleotide, the circular polynucleotide, or the expression vector of any aspect provided herein, or embodiments thereof.

In another aspect, the disclosure features a polynucleotide encoding an RNA molecule containing one or more of the following:
(a) from 5′ to 3′: a first ribozyme, a first ligation sequence, a PP7 RNA hairpin, a second ligation sequence, and a second ribozyme;
(b) from 5′ to 3′: first ribozyme, a first ligation sequence, a PP7 RNA hairpin, an hCTE RNA hairpin, a second ligation sequence, and a second ribozyme;
(c) from 5′ to 3′: a first ribozyme, a first ligation sequence, a BC1 RNA hairpin, a second ligation sequence, and a 3′ ribozyme; or
(d) from 5′ to 3′: a first ribozyme, a first ligation sequence, a BC200 RNA hairpin, a second ligation sequence, and a second ribozyme.

In another aspect, the disclosure features a polynucleotide encoding from 5′ to 3′:
(a) a first ribozyme, a first ligation sequence, a PP7 RNA hairpin, a second ligation sequence, a second ribozyme, and PP7cp fused to a Far motif,
(b) a first ribozyme, a first ligation sequence, a PP7 RNA hairpin, an hCTE RNA hairpin, a second ligation sequence, a second ribozyme, and PP7cp fused to an M9 tag and a nuclear export signal (NES);
(c) a first ribozyme, a first ligation sequence, a PP7 RNA hairpin, a second ligation sequence, a second ribozyme, and RNA 2′,3′-cyclic phosphate and 5′-OH ligase (RtcB) fused to three tandem repeats of a nuclear localization signal (NLS), a self-cleaving peptide, and PP7cp fused to a Far motif;
(d) a first ribozyme, a first ligation sequence, a PP7 RNA hairpin, a second ligation sequence, a second ribozyme, DDX39A, a self-cleaving peptide, and PP7cp fused to a Far motif;
(e) a first ribozyme, a first ligation sequence, a PP7 RNA hairpin, a second ligation sequence, a second ribozyme, and PP7cp fused to an M9 tag and a NES, a self-cleaving peptide, and PP7cp fused to a Far motif;
(f) a first ribozyme, a first ligation sequence, a PP7 RNA hairpin, an hCTE RNA hairpin, a second ligation sequence, a second ribozyme, and PP7cp fused to an M9 tag and a NES, a self-cleaving peptide, and PP7cp fused to a Far motif; or
(g) a first ribozyme, a first ligation sequence, a PP7 RNA hairpin, a second ligation sequence, a second ribozyme, and PP7cp fused to a Far motif.

In another aspect, the disclosure features a polynucleotide encoding from 5′ to 3′:
(a) a first ribozyme, a first ligation sequence, a PP7 RNA hairpin, a second ligation sequence, a second ribozyme, PP7cp fused to an M9 tag and a NES, a self-cleaving peptide, tdPP7cp fused VAMP2A;
(b) a first ribozyme, a first ligation sequence, a PP7 RNA hairpin, a second ligation sequence, a second ribozyme, PP7cp fused to an M9 tag and a NES, a self-cleaving peptide, SYP1 fused to tdPP7cp;
(c) a first ribozyme, a first ligation sequence, a MS2 RNA hairpin, a second ligation sequence, a second ribozyme, tandem MS2cp fused to homer1c;
(d) a first ribozyme, a first ligation sequence, a MS2 RNA hairpin, a second ligation sequence, a second ribozyme, MS2cp fused to an M9 tag and a NES, a self-cleaving peptide, a PSD95 fibronectin intrabody (FingR) polypeptide fused to tdMS2cp, CCR5TC, and KRAB;
(e) a first ribozyme, a first ligation sequence, a Box RNA hairpin, a second ligation sequence, a second ribozyme, λN fused to an M9 tag and a NES, a self-cleaving peptide, and a GPHN FingR polypeptide fused to λN, IL2RGTC, and KRAB; or
(f) a first ribozyme, a first ligation sequence, a Box RNA hairpin, a second ligation sequence, a second ribozyme, and ARC fused to λN.

In another aspect, the disclosure features an expression vector containing the polynucleotide of any aspect provided herein, or embodiments thereof, where the expression vector contains a U6 promoter that controls expression of the RNA polynucleotide.

In another aspect, the disclosure features a cell containing the polynucleotide or the expression vector of any aspect provided herein, or embodiments thereof.

In another aspect, the disclosure features a system for localizing a ribozyme-assisted circular RNA molecular to a cellular location. The system contains (a) a circular RNA molecule containing an RNA hairpin capable of binding an RNA binding domain and a heterologous polynucleotide. The system further contains (b) one or more fusion proteins containing the RNA binding domain and (i) a polypeptide domain that localizes to a cellular location of interest; or (ii) a nuclear export domain.

In another aspect, the disclosure features a polynucleotide encoding the system of any aspect provided herein, or embodiments thereof.

In another aspect, the disclosure features an expression vector containing the polynucleotide of any aspect provided herein, or embodiments thereof.

In another aspect, the disclosure features a cell containing the polynucleotide or the expression vector of any aspect provided herein, or embodiments thereof.

In another aspect, the disclosure features a method for characterizing a tissue of a subject. The method involves (a) contacting a cell with the polynucleotide of any aspect provided herein, or embodiments thereof, under conditions that permit expression of a circular RNA molecule encoded by the polynucleotide, where the circular RNA molecule contains a unique molecular identifier. The method further involves (b) determining localization of the circular RNA molecule within the cell using spatially-resolved transcript amplicon readout mapping.

In another aspect, the disclosure features a method for single cell morphological tracing. The method involves (a) contacting a cell in vivo or in vitro with a vector containing a polynucleotide encoding one or more RNA polynucleotides and one or more RNA binding polypeptides. Each RNA polynucleotide contains the following elements, each of which is operably linked: i) a first ribozyme; ii) a first ligation sequence; iii) an RNA hairpin sequence; iv) a heterologous polynucleotide containing a unique molecular identifier; v) a second ligation sequence; and vi) a second ribozyme. The RNA hairpin sequence specifically binds the RNA binding polypeptides. Also, each RNA binding polypeptide contains a domain that tethers the RNA binding polypeptide to a cellular membrane. The method further involves (b) detecting the unique molecular identifier in the cell, thereby tracing single cell morphology.

In another aspect, the disclosure features a method for characterizing viral tropism. The method involves (a) contacting a cell in vivo or in vitro with a viral vector containing a polynucleotide encoding one or more RNA polynucleotides and one or more RNA binding polypeptides. Each RNA polynucleotide contains the following elements, each of which is operably linked: i) a first ribozyme; ii) a first ligation sequence; iii) an RNA hairpin sequence; iv) a heterologous polynucleotide containing a unique molecular identifier; v) a second ligation sequence; and vi) a second ribozyme. The RNA hairpin sequence specifically binds the RNA binding polypeptides. Also, each RNA binding polypeptide contains a domain that tethers the RNA binding polypeptide to a cellular membrane. The method further involves, (b) detecting the unique molecular identifier in the cell, thereby characterizing tropism of the viral vector.

In another aspect, the disclosure features a method for mapping the connectome of a neuron cell. The method involves (a) contacting a neuron in vivo or in vitro with retrograde adenoviral associated viral (retroAAV) vector containing a polynucleotide encoding one or more RNA polynucleotides and one or more RNA binding polypeptides. Each RNA polynucleotide contains the following elements, each of which is operably linked: i) a first ribozyme; ii) a first ligation sequence; iii) an RNA hairpin sequence; iv) a heterologous polynucleotide containing a unique molecular identifier; v) a second ligation sequence; and vi) a second ribozyme. The RNA hairpin sequence specifically binds the RNA binding polypeptides. Also, each RNA binding polypeptide contains a domain that tethers the RNA binding polypeptide to a cellular membrane. The method further involves (b) detecting the unique molecular identifier in the cell, thereby mapping the connectome of the neuron.

In another aspect, the disclosure features a method for introducing a heterologous polynucleotide to the cytoplasm of a cell. The method involves (a) contacting the cell in vivo or in vitro with a vector containing a polynucleotide encoding one or more RNA polynucleotides and an RNA binding polypeptide. Each RNA polynucleotide contains the following elements, each of which is operably linked: i) a first ribozyme; ii) a first ligation sequence; iii) an RNA hairpin sequence; iv) a heterologous polynucleotide containing a heterologous polynucleotide; v) a second ligation sequence; and vi) a second ribozyme. The RNA hairpin sequence specifically binds the RNA binding polypeptide. Also, the RNA binding polypeptide mediates nuclear export.

In another aspect, the disclosure features a method for characterizing a tissue of a subject. The method involves (a) contacting an organism with an agent and a vector expressing a circular RNA barcode under conditions that permit expression of the RNA barcodes in a tissue of the subject. The method also involves (b) obtaining a biological sample from the subject and sectioning the sample to obtain tissue sections containing expressed RNA bar codes. The method further involves (c) contacting the tissue sections with a detectable probe containing a gene specific identifier and a region where a reading probe aligns to an endogenous gene to detect spatially resolved in situ endogenous gene sequence. The method further involves (d) contacting the tissue sections with a primer that hybridizes to a common region within the RNA barcode and a probe that hybridizes to a variable region within the RNA barcode to obtain a spatially resolved in situ RNA sequence. The sequence of (c) and the sequence of (d) are computationally integrated and detected at a nanometer voxel size. The method also involves (e) computationally analyzing the voxels to generate a molecularly defined cell-type and tissue region map containing a spatially resolved single-cell expression profile to obtain a comprehensive spatial cell atlas of the tissue.

In another aspect, the disclosure features a method for characterizing viral tropism in a tissue of a subject. The method involves (a) injecting a subject with an AAV vector expressing circular RNA barcodes under conditions that permit expression of the RNA barcodes in a tissue of the subject. The method also involves (b) obtaining a biological sample from the subject and sectioning the sample to obtain tissue sections. The method further involves (c) contacting the tissue sections with a detectable probe containing a gene specific identifier and a region where a reading probe aligns to detect spatially resolved in situ endogenous gene sequence. The method also involves (d) contacting the tissue sections with a primer that hybridizes to a common region within the RNA barcode and a probe that hybridizes to a variable region within the RNA barcode to obtain a spatially resolved in situ RNA sequence. The sequence of (c) and the sequence of (d) are detected at a nanometer voxel size. The method further involves (e) computationally analyzing the voxels to generate a molecularly defined cell-type and tissue region map containing spatially resolved single-cell expression profiles.

In another aspect, the disclosure features a method involving performing in situ sequencing of each tissue section of a plurality of tissue sections of a tissue to identify genes expressed at locations within each tissue section. The method also involves identifying individual cells present within each tissue section and labeling each individual cell with a cell type using the genes identified as being expressed at the locations within each tissue section. The method further involves storing information describing a three-dimensional structure of the tissue, the information describing the three-dimensional structure of the tissue containing locations within the tissue at which different cell types appear.

In another aspect, the disclosure features a method involving obtaining a reference structure for a reference sample of a tissue in a reference state, the reference structure identifying a gene expression of individual cells at locations in the reference sample of the tissue. The method also involves obtaining a second structure for a second sample of the tissue in a second state different from the reference state, the second structure identifying a gene expression of individual cells at locations in the second sample. The method further involves determining one or more differences in gene expression of individual cells between the reference state and the second state using the reference structure and the second structure. The method further involves outputting the one or more differences in the gene expression of individual cells.

In another aspect, the disclosure features a method involving determining information to output to a user regarding a composition of a tissue. The information regarding the composition of the tissue contains information indicating a location of individual cells within the tissue. The determining involves: filtering a data set of information regarding the tissue responsive to user-input filtering criteria, where the information regarding the tissue contains information on genes expressed in individual cells in the tissue and where the user-input filtering criteria identifies one or more genes for which information is to be output. The determining also involves selecting, for output to the user as part of the information regarding the composition of the tissue, information regarding cells detected to have expressed the one or more genes for which information is to be output, the information regarding the cells containing the location of the cells within the tissue. The method further involves outputting the information regarding the composition of the tissue for presentation to the user.

In another aspect, the disclosure features an RNA polynucleotide containing a sequence with at least 85% sequence identity to a sequence selected from one or more of:

a)
(SEQ ID NO: 1)
ccgcacUcgccggUcccaagcccggaUaaaaUgggagggggcgggaaaccgccUaaccaUgcc
gagUgcggccgcUUgccaUgUgUaUcggUccgacaUgaggaUcacccaUgUcggUccgaUacUc
UgaUgaU(Nₙ)gggUcccaUcaUUcaUggcaagUggccgcggUcggcgUggacUgUagaacacUg
ccaaUgccggUcccaagcccggaUaaaaGUGGAGGGUACAGUCCACGC (racRNA-MS2);
b)
(SEQ ID NO: 2)
gccgcacUcgccggUcccaagcccggaUaaaaUgggagggggcgggaaaccgccUaaccaUgc
cgagUgcggccgcUUgccaUgUgUaUcggUccgGGAGCAGACGAUAUGGCGUCGCUCCcggUcc
gaUacUcUgaUgaU(Nₙ)gggUcccaUcaUUcaUggcaagUggccgcggUcggcgUggacUgUag
aacacUgccaaUgccggUcccaagcccggaUaaaaGUGGAGGGUACAGUCCACGC
(racRNA-PP7);
c)
(SEQ ID NO: 3)
gccgcacUcgccggUcccaagcccggaUaaaaUgggagggggcgggaaaccgccUaaccaUgc
cgagUgcggccgcUUgccaUgUgUaUcggUccgcUUaagaaaaaaaaaggggUUggggaUUUag
cUcagUggUagagcgcUUgccUagcaagcgcaaggcccUgggUUcggUccUcagcUcUggaaaa
aaaaaaaaaaaaaaaaaaagacaaaaUaacaaaaagaccaaaaaaaaacaaggUaacUggcaca
cacaaccUUUaaaaaaaaagUUaaccggUccgaUacUcUgaUgaU(Nₙ)gggUcccaUcaUUcaU
ggcaagUggccgcggUcggcgUggacUgUagaacacUgccaaUgccggUcccaagcccggaUaa
aaGUGGAGGGUACAGUCCACGC (racRNA-BC1);
d)
(SEQ ID NO: 4)
cgacgggccgcacUcgccggUcccaagcccggaUaaaaUgggagggggcgggaaaccgccUaa
ccaUgccgagUgcggccgcUUgccaUgUgUaUcggUccgGGAGCAGACGAUAUGGCGUCGCUCC
cggUccgaUacUcUgaUgaU(Nn)CACUAACCUAAGACAGGAGGGCCGGGAAACCUGCCUAAUCC
AAUGACGGGUAAUAGUGgggacccaUcaUUcaUggcaagUggccgcggUcggcgUggacUgUag
aacacUgccaaUgccggUcccaagcccggaUaaaaGUGGAGGGUACAGUCCACGC
(racRNA-PP7-hCUE);
and
e)
(SEQ ID NO: 5)
gccgcacUcgccggUcccaagcccggaUaaaaUgggagggggcgggaaaccgccUaaccaUgc
cgagUgcggccgcUUgccaUgUgUaUcggUccgGGAGCAGACGAUAUGGCGUCGCUCCcggUcc
gaUacUcUgaUgaU(Nₙ)AAAAAAAAAAAAAAAAAAAAAAAAAAAAAAgggUcccaUcaUUcaUg
gcaagUggccgcggUcggcgUggacUgUagaacacUgccaaUgccggUcccaagcccggaUaaa
aGUGGAGGGUACAGUCCACGC (racRNA-PP7-30A);

where, N is any nucleotide and n is a number between 1 and 1000.

In another aspect, the disclosure features a vector encoding the RNA polynucleotide of any aspect provided herein, or embodiments thereof.

In any aspect provided herein, or embodiments thereof, the first and second ligation sequences are capable of hybridizing to one another.

In any aspect provided herein, or embodiments thereof, the RNA hairpin is selected from one or more of a BC1, BC200, BoxB, hCTE, MS2, and PP7.

In any aspect provided herein, or embodiments thereof, the heterologous polynucleotide contains a barcode, a unique molecular identifier, or a poly-A.

In any aspect provided herein, or embodiments thereof, the RNA polynucleotide further contains a second RNA hairpin containing an RNA element that mediates nuclear export. In any aspect provided herein, or embodiments thereof, the second RNA hairpin is hCTE.

In any aspect provided herein, or embodiments thereof, the RNA hairpin binds a viral coat protein. In any aspect provided herein, or embodiments thereof, the viral coat protein is PP7 coat protein (PP7cp). In any aspect provided herein, or embodiments thereof, the viral coat protein is MS2 coat protein (MS2cp). In any aspect provided herein, or embodiments thereof, the RNA binding polypeptide contains λN. In any aspect provided herein, or embodiments thereof, the RNA hairpin specifically binds a viral coat protein.

In any aspect provided herein, or embodiments thereof, the RNA binding polypeptide is an RNA export receptor. In any aspect provided herein, or embodiments thereof, the RNA export receptor is selected from one or more of CRM1, NXF1, DDX39A, or DDX39B.

In any aspect provided herein, or embodiments thereof, the ligation sequences are suitable for ligation to one another using an RNA ligase or a tRNA processing ligase.

In any aspect provided herein, or embodiments thereof, the vector further contains a promoter.

In any aspect provided herein, or embodiments thereof, the circular RNA polynucleotide further contains a second RNA hairpin.

In any aspect provided herein, or embodiments thereof, the RNA molecule further contains a heterologous polynucleotide that is 3′ of the first ligation sequence and 5′ of the second ligation sequence. In any aspect provided herein, or embodiments thereof, the heterologous polynucleotide contains a barcode and/or a unique molecular identifier.

In any aspect provided herein, or embodiments thereof, the polynucleotide further contains 10-60 consecutive adenosines (SEQ ID NO: 6). In any aspect provided herein, or embodiments thereof, the polynucleotide further contains 30 consecutive adenosines (SEQ ID NO: 7). In any aspect provided herein, or embodiments thereof, the consecutive adenosines are 3′ of the RNA hairpin. In any aspect provided herein, or embodiments thereof, the consecutive adenosines are adjacent to and 3′ of the heterologous polynucleotide.

In any aspect provided herein, or embodiments thereof, the polynucleotide further contains a heterologous sequence encoding a polypeptide. In any aspect provided herein, or embodiments thereof, the polypeptide contains an RNA binding polypeptide. In any aspect provided herein, or embodiments thereof, the RNA binding polypeptide is selected from one or more of PP7cp, MS2cp, and λN. In any aspect provided herein, or embodiments thereof, the polypeptide further contains a nuclear export domain. In any aspect provided herein, or embodiments thereof, the nuclear export domain contains an M9 tag and a nuclear export signal. In any aspect provided herein, or embodiments thereof, the polypeptide contains a membrane anchoring motif In any aspect provided herein, or embodiments thereof, the membrane anchoring motif is a farnesylation (Far) motif. In any aspect provided herein, or embodiments thereof, the polypeptide contains an RNA ligase. In any aspect provided herein, or embodiments thereof, the RNA ligase is RNA 2′,3′-cyclic phosphate and 5′-OH ligase (RtcB). In any aspect provided herein, or embodiments thereof, the polypeptide further contains a nuclear localization signal (NLS). In any aspect provided herein, or embodiments thereof, the polypeptide contains three or more tandem nuclear localization signals. In any aspect provided herein, or embodiments thereof, the polypeptide contains a DDX39A polypeptide. In any aspect provided herein, or embodiments thereof, the polypeptide contains an epitope tag. In any aspect provided herein, or embodiments thereof, the epitope tag is selected from one or more of a FLAG tag, an HA tag, and a V5 tag. In any aspect provided herein, or embodiments thereof, the polypeptide contains a fluorescent polypeptide. In any aspect provided herein, or embodiments thereof, the polypeptide contains a VAMP2A polypeptide, a SYP1 polypeptide, a homer1c polypeptide, a CCR5TC domain fused to a KRAB domain, a IL2RGTC domain fused to a KRAB domain, a PSD95 FingR domain, a GPHN FingR domain, an ARC polypeptide, a tandem PP7cp polypeptide, or a tandem MS2cp polypeptide. In any aspect provided herein, or embodiments thereof, the polypeptide contains two or more polypeptide molecules linked to one another by a self-cleaving peptide. In any aspect provided herein, or embodiments thereof, the self-cleaving peptide is T2A.

In any aspect provided herein, or embodiments thereof, the polynucleotide further contains a promoter controlling expression of the RNA molecule or a polypeptide encoded by the polynucleotide. In any aspect provided herein, or embodiments thereof, the promoter is a constitutive promoter. In any aspect provided herein, or embodiments thereof, the promoter is selectively expressed in a target cell. In any aspect provided herein, or embodiments thereof, the polypeptide encoded by the polynucleotide is expressed under the control of a CAG promoter, hSyn promoter, or TRE promoter.

In any aspect provided herein, or embodiments thereof, the polynucleotide further contains a binding site for CCR5TC-KRAB or IL2RGTC-KRAB upstream of the promoter controlling expression of the RNA molecule, and where binding of the CCR5TC-KRAB or IL2RGTC-KRAB to the binding site represses expression of the RNA molecule.

In any aspect provided herein, or embodiments thereof, the vector is an adeno-associated virus (AAV) vector. In any aspect provided herein, or embodiments thereof, the AAV vector has the serotype AAV-PHP.eB. In any aspect provided herein, or embodiments thereof, the AAV vector is a retroAAV vector.

In any aspect provided herein, or embodiments thereof, the cell is a neuron.

In any aspect provided herein, or embodiments thereof, the RNA hairpin is selected from one or more of a BC1, BC200, BoxB, hCTE, MS2, PP7.

In any aspect provided herein, or embodiments thereof, the circular RNA molecule contains two or more RNA hairpins capable of binding an RNA binding domain. In any aspect provided herein, or embodiments thereof, the circular RNA molecule contains a PP7 RNA hairpin and an hCTE RNA hairpin.

In any aspect provided herein, or embodiments thereof, the RNA binding domain contains a PP7 coat protein, an MS2 coat protein, or λN.

In any aspect provided herein, or embodiments thereof, the polypeptide that localizes to a cellular location of interested is selected from one or more of a VAMP2A polypeptide, a SYP1 polypeptide, a homer1c polypeptide, a CCR5TC domain fused to a KRAB domain, a IL2RGTC domain fused to a KRAB domain, and an ARC polypeptide. In any aspect provided herein, or embodiments thereof, the polypeptide that localizes to a cellular location of interest is a membrane anchoring motif. In any aspect provided herein, or embodiments thereof, the membrane anchoring motif is a farnesylation (Far) motif.

In any aspect provided herein, or embodiments thereof, the nuclear export domain contains an M9 tag. In any aspect provided herein, or embodiments thereof, the nuclear export domain contains an M9 tag and a nuclear export signal (NES).

In any aspect provided herein, or embodiments thereof, the circular RNA molecule is encoded by the polynucleotide of any aspect provided herein, or embodiments thereof.

In any aspect provided herein, or embodiments thereof, the system contains both (a) a fusion protein containing the RNA binding polypeptide domain and a polypeptide domain that localizes to a cellular compartment of interest and (b) another fusion protein containing the RNA binding polypeptide domain and an RNA shuttling domain.

In any aspect provided herein, or embodiments thereof, the vector is a viral vector. In any aspect provided herein, or embodiments thereof, the vector is an adeno-associated virus (AAV) vector. In any aspect provided herein, or embodiments thereof, the AAV vector has the serotype AAV-PHP.eB. In any aspect provided herein, or embodiments thereof, the vector is a retroAAV vector.

In any aspect provided herein, or embodiments thereof, the cell is a neuron.

In any aspect provided herein, or embodiments thereof, the domain tethers the RNA binding polypeptide to a cellular location. In any aspect provided herein, or embodiments thereof, the domain tethers the RNA binding polypeptide to a cell membrane.

In any aspect provided herein, or embodiments thereof, the RNA binding polypeptide contains an epitope tag.

In any aspect provided herein, or embodiments thereof, the unique molecular identifier is detectable in imaging. In any aspect provided herein, or embodiments thereof, the unique molecular identifier is detected by sequencing.

In any aspect provided herein, or embodiments thereof, the polynucleotide contains a U6 promoter that controls expression of the one or more RNA polynucleotides.

In any aspect provided herein, or embodiments thereof, the unique molecular identifier is detected using STARmap.

In any aspect provided herein, or embodiments thereof, the method further involves quantifying RNA molecule copy numbers in individual cells.

In any aspect provided herein, or embodiments thereof, the viral vector is an adeno associated viral vector.

In any aspect provided herein, or embodiments thereof, where the unique molecular identifier is an RNA barcode, and where the method further involves sequencing a cellular transcriptome and the RNA barcode in the cell in a tissue sample, thereby characterizing a cell-type-resolved tropism of the viral vector.

In any aspect provided herein, or embodiments thereof, the cell is in a subject. In any aspect provided herein, or embodiments thereof, the cell is in a tissue of the subject. In any aspect provided herein, or embodiments thereof, the tissue is a brain tissue. In any aspect provided herein, or embodiments thereof, the subject is a mammal. In any aspect provided herein, or embodiments thereof, the mammal is a rodent. In any aspect provided herein, or embodiments thereof, the mammal is a human.

In any aspect provided herein, or embodiments thereof, RNA polynucleotide forms a circular RNA molecule that localizes to a subcellular compartment of the cell. In any aspect provided herein, or embodiments thereof, the subcellular compartment contains the nucleus, the soma, the cytoplasm, neurites, and/or dendrites.

In any aspect provided herein, or embodiments thereof, the method characterizes the morphology or lineage of the cell.

In any aspect provided herein, or embodiments thereof, the heterologous polypeptide is complementary to an RNA molecule present in the cytoplasm of the cell.

In any aspect provided herein, or embodiments thereof, the tissue is the central nervous system. In any aspect provided herein, or embodiments thereof, the subject is a rodent or primate.

In any aspect provided herein, or embodiments thereof, the agent is a therapeutic agent. In any aspect provided herein, or embodiments thereof, the therapeutic agent has neuropsychiatric activity. In any aspect provided herein, or embodiments thereof, the agent is a serotonin reuptake inhibitor.

In any aspect provided herein, or embodiments thereof, the method further involves comparing the spatially resolved single-cell expression profile of (e) to a reference spatially resolved single-cell expression profile.

In any aspect provided herein, or embodiments thereof, the circular RNA barcode is expressed under the control of a U6 promoter.

In any aspect provided herein, or embodiments thereof, the expression profile contains 100 million to 500 million RNA reads. In any aspect provided herein, or embodiments thereof, the method characterizes the expression profile or 500 hundred thousand to 2 million cells.

In any aspect provided herein, or embodiments thereof, the method further involves computationally integrating cell morphological data, nuclear staining data, or cell type data.

In any aspect provided herein, or embodiments thereof, the cell type data characterizes the cell by neurotransmitter type.

In any aspect provided herein, or embodiments thereof, the method further involves computationally integrating heatmap data.

In any aspect provided herein, or embodiments thereof, the probe that binds to an endogenous gene is a SNAIL probe.

In any aspect provided herein, or embodiments thereof, the RNA barcode probe is a padlock probe.

In any aspect provided herein, or embodiments thereof, gene imputation is part of cell type identification.

In any aspect provided herein, or embodiments thereof, the vector further contains a polynucleotide encoding a polypeptide with at least 85% sequence identity to an amino acid sequence selected from one or more of:

a)
(SEQ ID NO: 8)
MASNFTQFVLVDNGGTGDVTVAPSNFANGVAEWISSNSRSQAYKVTCSVRQSSAQNRKYTIKV
EVPKGAWRSYLNMELTIPIFSTNSDCELIVKAMQGLLKDGNPIPSAIAANSGIYGGGGSGGGGS
NDFGNYNNQSSNFGPMKGGNFGGRSSGPYGGGGQYFAKPRNQGGYGGGGSLPPLERLTLGSGGS
GGSEGRGSLLTCGDVEENPGPATMLEVKEASPTSIQISWVLHLRHVRYYRITYGETGGNSPVQE
FTVPGSKSTATISGLKPGVDYTITVYAVTIFSAYRSAWPPISINYRTGTDYKDDDDKGSGSSRS
GLLKATMASNFTQFVLVDNGGTGDVTVAPSNFANGVAEWISSNSRSQAYKVTCSVRQSSAQKRK
YTIKVEVPKVATQTVGGVELPVAARRSYLNMELTIPIFATNSDCELIVKAMQGLLKDGNPIPSA
IAANSGIYGAPGIHPGMMASNFTQFVLVDNGGTGDVTVAPSNFANGVAEWISSNSRSQAYKVTC
SVRQSSAQKRKYTIKVEVPKVATQTVGGVELPVAARRSYLNMELTIPIFATNSDCELIVKAMQG
LLKDGNPIPSAIAANSGIY (MS2cp-M9-NES-T2A-PSD95.FingR-FLAG-tdMS2cp);
b)
(SEQ ID NO: 9)
MSKTIVLSVGEATRTLTEIQSTADRQIFEEKVGPLVGRLRLTASLRQNGAKTAYRVNLKLDQA
DVVDCSTSVCGELPKVRYTQVWSHDVTIVANSTEASRKSLYDLTKSLVATSQVEDLVVNLVPLG
RGGGGSGGGGSNDFGNYNNQSSNFGPMKGGNFGGRSSGPYGGGGQYFAKPRNQGGYGGGGSLPP
LERLTLGSGGSGGSEGRGSLLTCGDVEENPGPATMSKTIVLSVGEATRTLTEIQSTADRQIFEE
KVGPLVGRLRLTASLRQNGAKTAYRVNLKLDQADVVDCSTSVCGELPKVRYTQVWSHDVTIVAN
STEASRKSLYDLTKSLVATSQVEDLVVNLVPLGRRADPLASCGRSKTIVLSVGEATRTLTEIQS
TADRQIFEEKVGPLVGRLRLTASLRQNGAKTAYRVNLKLDQADVVDCSTSVCGELPKVRYTQVW
SHDVTIVANSTEASRKSLYDLTKSLVATSQVEDLVVNLVPLGRRADPLASTRDSTSGGSGGGYP
YDVPDYASGGGSGGGMSATAATVPPAAPAGEGGPPAPPPNLTSNRRLQQTQAQVDEVVDIMRVN
VDKVLERDQKLSELDDRADALQAGASQFETSAAKLKRKYWWKNLKMMIILGVICAIILIIIIVY
FST (PP7cp-M9-NES-T2A-tdPP7cp-VAMP2A);
c)
(SEQ ID NO: 10)
MGKPIPNPLLGLDSTGGGGSSKTIVLSVGEATRTLTEIQSTADRQIFEEKVGPLVGRLRLTAS
LRQNGAKTAYRVNLKLDQADVVDCSTSVCGELPKVRYTQVWSHDVTIVANSTEASRKSLYDLTK
SLVATSQVEDLVVNLVPLGRGGGGSGGGGSNDFGNYNNQSSNFGPMKGGNFGGRSSGPYGGGGQ
YFAKPRNQGGYGGGGSLPPLERLTLGSGGSGGSEGRGSLLTCGDVEENPGPAMDYKDDDDKGGG
GSSKTIVLSVGEATRTLTEIQSTADRQIFEEKVGPLVGRLRLTASLRQNGAKTAYRVNLKLDQA
DVVDCSTSVCGELPKVRYTQVWSHDVTIVANSTEASRKSLYDLTKSLVATSQVEDLVVNLVPLG
RGGGGSKLNPPDESGPGCMSCCVLS (V5-PP7cp-M9-NES-T2A-FLAG-PP7cp-Far);
d)
(SEQ ID NO: 11)
MGKPIPNPLLGLDSTGGGGSSKTIVLSVGEATRTLTEIQSTADRQIFEEKVGPLVGRLRLTAS
LRQNGAKTAYRVNLKLDQADVVDCSTSVCGELPKVRYTQVWSHDVTIVANSTEASRKSLYDLTK
SLVATSQVEDLVVNLVPLGRGGGGSGGGGSNDFGNYNNQSSNFGPMKGGNFGGRSSGPYGGGGQ
YFAKPRNQGGYGGGGSLPPLERLTLGSGGSGGSEGRGSLLTCGDVEENPGPAMVSKGEEDNMAI
IKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKL
DITSHNEDYTIVEQYERAEGRHSTGGMDELYKGGGGSSKTIVLSVGEATRTLTEIQSTADRQIF
EEKVGPLVGRLRLTASLRQNGAKTAYRVNLKLDQADVVDCSTSVCGELPKVRYTQVWSHDVTIV
ANSTEASRKSLYDLTKSLVATSQVEDLVVNLVPLGRGGGGSKLNPPDESGPGCMSCCVLS
(V5tag-PP7cp-M9-NES-T2A-mCherry-PP7cp-Far);
e)
(SEQ ID NO: 12)
VSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVK
LRGTNFPSDGPVMQKKTMGWEASSERMYPEDGALKGEIKQRLKLKDGGHYDAEVKTTYKAKKPV
QLPGAYNVNIKLDITSHNEDYTIVEQYERAEGRHSTGGMDELYK (mCherry);
f)
(SEQ ID NO: 13)
SKTIVLSVGEATRTLTEIQSTADRQIFEEKVGPLVGRLRLTASLRQNGAKTAYRVNLKLDQAD
VVDCSTSVCGELPKVRYTQVWSHDVTIVANSTEASRKSLYDLTKSLVATSQVEDLVVNLVPLGR
GGGGSGGGGSNDFGNYNNQSSNFGPMKGGNFGGRSSGPYGGGGQYFAKPRNQGGYGGGGSLPPL
ERLTL (PP7cp-M9-NES);
g)
(SEQ ID NO: 14)
SKTIVLSVGEATRTLTEIQSTADRQIFEEKVGPLVGRLRLTASLRQNGAKTAYRVNLKLDQAD
VVDCSTSVCGELPKVRYTQVWSHDVTIVANSTEASRKSLYDLTKSLVATSQVEDLVVNLVPLGR
GGGGSKLNPPDESGPGCMSCCVLS (PP7cp-Far);
and
h)
(SEQ ID NO: 15)
MASNFTQFVLVDNGGTGDVTVAPSNFANGVAEWISSNSRSQAYKVTCSVRQSSAQNRKYTIKV
EVPKGAWRSYLNMELTIPIFSTNSDCELIVKAMQGLLKDGNPIPSAIAANSGIYGGGGSGGGGS
NDFGNYNNQSSNFGPMKGGNFGGRSSGPYGGGGQYFAKPRNQGGYGGGGSLPPLERLTL
(MS2cp-M9-NES).

In any aspect of the disclosure, or embodiments thereof, the polynucleotide comprises a nucleotide sequence with at least about 85% sequence identity to a sequence listed in Table 1A or Table 3. In any aspect of the disclosure, or embodiments thereof, the polypeptide contains or the polynucleotide encodes an amino acid sequence with at least about 85% sequence identity to a sequence listed in Table 4.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound. In embodiments, an agent is a circular RNA.

By “ameliorate” is meant decrease, suppress, attenuate, diminish, arrest, or stabilize the development or progression of a disease.

The term “adaptor” refers to a sequence that is added, for example by ligation, to a nucleic acid. The length of an adaptor may be from about 5 to about 100 bases and may provide a sequencing primer binding site (e.g., an amplification primer binding site), and a molecular barcode such as a sample identifier sequence or molecule identifier sequence, preferably a unique identifier sequence. An adaptor may be added to 1) the 5′ end, 2) the 3′ end, or 3) both ends of a nucleic acid molecule. Double-stranded adaptors contain a double-stranded end ligated to a nucleic acid. An adaptor can have an overhang or may be blunt ended. As will be described in greater detail below, a double stranded adaptor can be added to a fragment by ligating only one strand of the adaptor to the fragment. The sequence of the non-ligated strand of the adaptor may be added to the fragment using a polymerase. Y-adaptors and loop adaptors are type of double-stranded adaptors.

By “alteration” is meant a change (increase or decrease) in the expression levels, structure, or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “amplicon” is meant a polynucleotide that is a product of amplification.

As used herein, the term “antisense strand” refers to a polynucleotide that is substantially or 100% complementary to a target nucleic acid of interest. For example, an antisense strand may be complementary, in whole or in part, to a molecule of mRNA (messenger RNA), an RNA sequence that is not mRNA (e.g., microRNA, piwiRNA, tRNA, rRNA and hnRNA) or a sequence of DNA that is either coding or non-coding.

By “activity-regulated cytoskeleton-associated protein (ARC) polypeptide” is meant a polypeptide, or fragment thereof, having at least about 85% amino acid sequence identity to NCBI Ref. Seq. Accession No. NP_001399781.1, which is provided below, and capable of mediating localization of a polypeptide to dendritic spines, or pan-dendritic compartments of a cell.
NP 001399781.1 activity-regulated cytoskeleton-associated protein [Homo sapiens]

(SEQ ID NO: 16)
MELDHRTSGGLHAYPGPRGGQVAKPNVILQIGKCRAEMLEHVRRTHRHLLAEVSKQVERELKGL
HRSVGKLESNLDGYVPTSDSQRWKKSIKACLCRCQETIANLERWVKREMHVWREVFYRLERWAD
RLESTGGKYPVGSESARHTVSVGVGGPESYCHEADGYDYTVSPYAITPPPAAGELPGQEPAEAQ
QYQPWVPGEDGQPSPGVDTQIFEDPREFLSHLEEYLRQVGGSEEYWLSQIQNHMNGPAKKWWEF
KQGSVKNWVEFKKEFLQYSEGTLSREAIQRELDLPQKQGEPLDQFLWRKRDLYQTLYVDADEEE
IIQYVVGTLQPKLKRFLRHPLPKTLEQLIQRGMEVQDDLEQAAEPAGPHLPVEDEAETLTPAPN
SESVASDRTQPE.

By “activity-regulated cytoskeleton-associated protein (ARC) polynucleotide” is meant a nucleic acid molecule encoding an ARC polypeptide. An exemplary ARC nucleotide sequence is provided below and at NCBI. Ref. Seq. Accession No. NM_001412852.1:209-1399.
NM_001412852.1:209-1399 Homo sapiens activity regulated cytoskeleton associated protein (ARC), transcript variant 2, mRNA

(SEQ ID NO: 17)
ATGGAGCTGGACCACCGGACCAGCGGCGGGCTCCACGCCTACCCCGGGCCGCGGGGGGGGCAGG
TGGCCAAGCCCAACGTGATCCTGCAGATCGGGAAGTGCCGGGCCGAGATGCTGGAGCACGTGCG
GCGGACGCACCGGCACCTGCTGGCCGAGGTGTCCAAGCAGGTGGAGCGCGAGCTGAAGGGGCTG
CACCGGTCGGTCGGGAAGCTGGAGAGCAACCTGGACGGCTACGTGCCCACGAGCGACTCGCAGC
GCTGGAAGAAGTCCATCAAGGCCTGCCTGTGCCGCTGCCAGGAGACCATCGCCAACCTGGAGCG
CTGGGTCAAGCGCGAGATGCACGTGTGGCGCGAGGTGTTCTACCGCCTGGAGCGCTGGGCCGAC
CGCCTGGAGTCCACGGGCGGCAAGTACCCGGTGGGCAGCGAGTCAGCCCGCCACACCGTTTCCG
TGGGCGTGGGGGGTCCCGAGAGCTACTGCCACGAGGCAGACGGCTACGACTACACCGTCAGCCC
CTACGCCATCACCCCGCCCCCAGCCGCTGGCGAGCTGCCCGGGCAGGAGCCCGCCGAGGCCCAG
CAGTACCAGCCGTGGGTCCCCGGCGAGGACGGGCAGCCCAGCCCCGGCGTGGACACGCAGATCT
TCGAGGACCCTCGAGAGTTCCTGAGCCACCTAGAGGAGTACTTGCGGCAGGTGGGCGGCTCTGA
GGAGTACTGGCTGTCCCAGATCCAGAATCACATGAACGGGCCGGCCAAGAAGTGGTGGGAGTTC
AAGCAGGGCTCCGTGAAGAACTGGGTGGAGTTCAAGAAGGAGTTCCTGCAGTACAGCGAGGGCA
CGCTGTCCCGAGAGGCCATCCAGCGCGAGCTGGACCTGCCGCAGAAGCAGGGCGAGCCGCTGGA
CCAGTTCCTGTGGCGCAAGCGGGACCTGTACCAGACGCTCTACGTGGACGCGGACGAGGAGGAG
ATCATCCAGTACGTGGTGGGCACCCTGCAGCCCAAGCTCAAGCGTTTCCTGCGCCACCCCCTGC
CCAAGACCCTGGAGCAGCTCATCCAGAGGGGCATGGAGGTGCAGGATGACCTGGAGCAGGCGGC
CGAGCCGGCCGGCCCCCACCTCCCGGTGGAGGATGAGGCGGAGACCCTCACGCCCGCCCCCAAC
AGCGAGTCCGTGGCCAGTGACCGGACCCAGCCCGAGTAG.

By “barcode” is meant a nucleic acid sequence that uniquely identifies polynucleotide molecules to which it is fused.

By “brain cytoplasmic RNA 1 (BC1) polynucleotide” is meant a nucleic acid molecule, or fragment thereof, having at least 85% sequence identity to NCBI Reference Sequence: NR_038088.1, and capable of facilitating transport of a polynucleotide molecule out of a cell nucleus. An exemplary BC1 non-coding RNA sequence is provided below:

(SEQ ID NO: 18)
GGGGTTGGGGATTTAGCTCAGTGGTAGAGCGCTTGCCTAGCAAGCGCAAG
GCCCTGGGTTCGGTCCTCAGCTCTGGAAAAAAAAAAAAAAAAAAAAAAAG
ACAAAATAACAAAAAGACCAAAAAAAAACAAGGTAACTGGCACACACAAC
CTTT.

By “BC200 polynucleotide” or “Homo sapiens brain cytoplasmic RNA 1 (BCYRN1)” is meant a nucleic acid molecule, or fragment thereof, having at least 85% sequence identity to NCBI Reference Sequence: NR_001568.1 and capable of facilitating transport of a polynucleotide molecule out of a cell nucleus. An exemplary polynucleotide sequence follows:

(SEQ ID NO: 19)
GGCCGGGCGCGGTGGCTCACGCCTGTAATCCCAGCTCTCAGGGAGGCTAA
GAGGCGGGAGGATAGCTTGAGCCCAGGAGTTCGAGACCTGCCTGGGCAAT
ATAGCGAGACCCCGTTCTCCAGAAAAAGGAAAAAAAAAAACAAAAGACAA
AAAAAAAATAAGCGTAACTTCCCTCAAAGCAACAACCCCCCCCCCCCTT
T.

By “BoxB polynucleotide” is meant an RNA hairpin that mediates binding to λN polypeptide. An exemplary BoxB hairpin nucleotide sequence follows: GGCCCTGAAAAAGGGCC (SEQ ID NO: 20). BoxB hairpins are described, for example, by Vieu et al., Journal of Molecular Biology, Volume 339, Issue 5, 18 Jun. 2004, Pages 1077-1087.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior Art embodiments.

By “complementary” is meant capable of pairing to form a double-stranded nucleic acid molecule or portion thereof. In one embodiment, an antisense molecule is in large part complementary to a target sequence. The complementarity need not be perfect, but may include mismatches at 1, 2, 3, or more nucleotides.

By “DexD-Box Helicase 39A (DDX39A) polypeptide” is meant a polypeptide, or fragment thereof, having at least about 85% amino acid sequence identity to NCBI Ref. Seq. Accession No. NP_005795.2 and having RNA helicase activity or having nuclear transport activity. An exemplary amino acid sequence follows:

(SEQ ID NO: 21)
MAEQDVENDLLDYDEEEEPQAPQESTPAPPKKDIKGSYVSIHSSGFRDFL
LKPELLRAIVDCGFEHPSEVQHECIPQAILGMDVLCQAKSGMGKTAVFVL
ATLQQIEPVNGQVTVLVMCHTRELAFQISKEYERFSKYMPSVKVSVFFGG
LSIKKDEEVLKKNCPHVVVGTPGRILALVRNRSFSLKNVKHFVLDECDKM
LEQLDMRRDVQEIFRLTPHEKQCMMFSATLSKDIRPVCRKFMQDPMEVFV
DDETKLTLHGLQQYYVKLKDSEKNRKLFDLLDVLEFNQVIIFVKSVQRCM
ALAQLLVEQNFPAIAIHRGMAQEERLSRYQQFKDFQRRILVATNLFGRGM
DIERVNIVFNYDMPEDSDTYLHRVARAGRFGTKGLAITFVSDENDAKILN
DVQDRFEVNVAELPEEIDISTYIEQSR.

By “DexD-Box Helicase 39A (DDX39A) polynucleotide” is meant a nucleic acid molecule encoding a DDX39A polypeptide. An exemplary DDX39A nucleotide sequence is provided below and at NCBI. Ref. Seq. Accession No. NM_005804.4.

(SEQ ID NO: 22)
GCAGAACAGGATGTGGAAAACGATCTTTTGGATTACGATGAAGAGGAAGA
GCCCCAGGCTCCTCAAGAGAGCACACCAGCTCCCCCTAAGAAAGACATCA
AGGGATCCTACGTTTCCATCCACAGCTCTGGCTTCCGGGACTTTCTGCTG
AAGCCGGAGCTCCTGCGGGCCATCGTGGACTGTGGCTTTGAGCATCCTTC
TGAGGTCCAGCATGAGTGCATTCCCCAGGCCATCCTGGGCATGGACGTCC
TGTGCCAGGCCAAGTCCGGGATGGGCAAGACAGCGGTCTTCGTGCTGGCC
ACCCTACAGCAGATTGAGCCTGTCAACGGACAGGTGACGGTCCTGGTCAT
GTGCCACACGAGGGAGCTGGCCTTCCAGATCAGCAAGGAATATGAGCGCT
TTTCCAAGTACATGCCCAGCGTCAAGGTGTCTGTGTTCTTCGGTGGTCTC
TCCATCAAGAAGGATGAAGAAGTGTTGAAGAAGAACTGTCCCCATGTCGT
GGTGGGGACCCCGGGCCGCATCCTGGCGCTCGTGCGGAATAGGAGCTTCA
GCCTAAAGAATGTGAAGCACTTTGTGCTGGACGAGTGTGACAAGATGCTG
GAGCAGCTGGACATGCGGCGGGATGTGCAGGAGATCTTCCGCCTGACACC
ACACGAGAAGCAGTGCATGATGTTCAGCGCCACCCTGAGCAAGGACATCC
GGCCTGTGTGCAGGAAGTTCATGCAGGATCCAATGGAGGTGTTTGTGGAC
GACGAGACCAAGCTCACGCTGCACGGCCTGCAGCAGTACTACGTCAAACT
CAAAGACAGTGAGAAGAACCGCAAGCTCTTTGATCTCTTGGATGTGCTGG
AGTTTAACCAGGTGATAATCTTCGTCAAGTCAGTGCAGCGCTGCATGGCC
CTGGCCCAGCTCCTCGTGGAGCAGAACTTCCCGGCCATCGCCATCCACCG
GGGCATGGCCCAGGAGGAGCGCCTGTCACGCTATCAGCAGTTCAAGGATT
TCCAGCGGCGGATCCTGGTGGCCACCAATCTGTTTGGCCGGGGGATGGAC
ATCGAGCGAGTCAACATCGTCTTTAACTACGACATGCCTGAGGACTCGGA
CACCTACCTGCACCGGGTGGCCCGGGCGGGTCGCTTTGGCACCAAAGGCC
TAGCCATCACTTTTGTGTCTGACGAGAATGATGCCAAAATCCTCAATGAC
GTCCAGGACCGGTTTGAAGTTAATGTGGCAGAACTTCCAGAGGAAATCGA
CATCTCCACATACATCGAGCAGAGCCGG.

By “decreases” is meant a reduction by at least about 5% relative to a reference level. A decrease may be by 5%, 10%, 15%, 20%, 25% or 50%, or even by as much as 75%, 85%, 95% or more and any intervening percentages

“Detect” refers to identifying the presence, absence, or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ.

The term “expression” or “expressed” as used herein in reference to a gene means the production of a transcriptional and/or translational product of that gene. The level of expression of a DNA molecule in a cell may be determined based on either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “famesylation (Far) motif peptide” or “famesylation (Far) motif” is meant an amino acid sequence that is modified by a famesyl transferase. In an embodiment, the Far motif comprises the sequence CaaX, where “C” is cysteine, each “a” is an aliphatic amino acid, and “X” is any amino acid. In various instances, the Far motif is located at the C-terminus of a polypeptide to which the Far motif is fused. In an embodiment, a Far motif has at least about 85% amino acid sequence identity to the following amino acid sequence: KLNPPDESGPGCMSCCVLS (SEQ ID NO: 23), or a fragment thereof. In an embodiment, a Far motif is fused to a protein of interest and mediates localization of the protein to a cell membrane.

By “famesylation (Far) motif polynucleotide” is meant a nucleic acid molecule encoding a Far motif. An exemplary Far nucleotide sequence is provided below.

(SEQ ID NO: 24)
AAGCTGAACCCTCCTGATGAGAGTGGCCCCGGCTGCATGAGCTGCTGTGT
GCTCTCC.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “Chain H, constitutive transport element (hCTE) RNA hairpin” is meant a nucleic acid molecule, or a fragment thereof, having at least 85% sequence identity to the following nucleotide sequence: CACTAACCTAAGACAGGAGGGCCGGGAAACCTGCCTAATCCAATGACGGGTAATAGTG (SEQ ID NO: 25) and capable of facilitating transport of a polynucleotide molecule out of a cell nucleus. An exemplary hCTE nucleic acid sequence is provided at PDB Accession No. 3RW6_H.

By “G domain of Gephyrin Fibronectin Intrabodies Generated with mRNA Display (GPHN.FingR) polypeptide” is meant a polypeptide, or fragment thereof, having at least about 85% amino acid sequence identity to the following sequence: MLEVKEASPTSIQISWGKYKVMVRYYRITYGETGGNSPVQEFTVPGSKSTATISSLKPGVDYTI TVYAVTIDHWNYQDPIPISINYRTGS (SEQ ID NO: 26) and capable of mediating localization of a polypeptide to an inhibitory post-synapse compartment of a cell. GPHN.FingR is described in Gross, G., et al., Neuron., 78:971-985, the disclosure of which is incorporated herein by reference in its entirety for all purposes.

By “G domain of Gephyrin Fibronectin Intrabodies Generated with mRNA Display (GPHN.FingR) polynucleotide” is meant a nucleic acid molecule encoding a GPHN.FingR polypeptide. An exemplary GPHN.FingR nucleotide sequence is provided below.

(SEQ ID NO: 27)
ATGCTCGAAGTCAAGGAAGCATCACCAACCAGCATCCAGATCAGCTGGGG
CAAGTACAAGGTCATGGTTCGCTACTACCGCATCACCTACGGTGAAACTG
GTGGCAATAGCCCTGTCCAGGAATTCACCGTGCCTGGCAGCAAGTCCACT
GCTACCATCAGCAGCCTGAAACCTGGTGTCGACTATACCATCACGGTGTA
CGCCGTCACGATCGACCACTGGAACTACCAGGACCCGATCCCGATCTCCA
TCAACTACCGCACCGGATCC.

By “homer protein homolog 1c (homer1c) polypeptide” is meant a polypeptide, or fragment thereof, having at least about 85% amino acid sequence identity to UniProtKB/Sqiss-Prot Seq. Accession No. Q9Z214, which is provided below, and capable of functioning as a post-synaptic marker protein.
spjQ9Z214.2|HOME1_RAT RecName: Full=Homer protein homolog 1; AltName: Full=PSD-Zip45; AltName: Full=VASP/Ena-related gene up-regulated during seizure and LTP 1; Short=Vesl-1

(SEQ ID NO: 28)
MGEQPIFSTRAHVFQIDPNTKKNWVPTSKHAVTVSYFYDSTRNVYRIISL
DGSKAIINSTITPNMTFTKTSQKFGQWADSRANTVYGLGFSSEHHLSKFA
EKFQEFKEAARLAKEKSQEKMELTSTPSQESAGGDLQSPLTPESINGTDD
ERTPDVTQNSEPRAEPAQNALPFSHSAGDRTQGLSHASSAISKHWEAELA
TLKGNNAKLTAALLESTANVKQWKQQLAAYQEEAERLHKRVTELECVSSQ
ANAVHSHKTELSQTVQELEETLKVKEEEIERLKQEIDNARELQEQRDSLT
QKLQEVEIRNKDLEGQLSELEQRLEKSQSEQDAFRSNLKTLLEILDGKIF
ELTELRDNLAKLLECS.

By “homer protein homolog 1c (homer1c) polynucleotide” is meant a nucleic acid molecule encoding a homer1c polypeptide. An exemplary homer1c nucleotide sequence is provided below.

(SEQ ID NO: 29)
ATGGGGGAACAACCTATCTTCAGCACTCGAGCTCATGTCTTCCAGATCGA
CCCAAACACAAAGAAGAACTGGGTACCCACCAGCAAGCATGCAGTTACTG
TGTCTTATTTCTATGACAGCACAAGGAATGTGTATAGGATAATCAGTTTA
GACGGCTCAAAGGCAATAATAAATAGCACCATCACTCCAAACATGACATT
TACTAAAACATCTCAAAAGTTTGGCCAATGGGCTGATAGCCGGGCAAACA
CTGTTTATGGACTGGGATTCTCCTCTGAGCATCATCTCTCAAAATTTGCA
GAAAAGTTTCAGGAATTTAAAGAAGCTGCTCGGCTGGCAAAGGAGAAGTC
GCAGGAGAAGATGGAACTGACCAGTACCCCTTCACAGGAATCAGCAGGAG
GAGATCTTCAGTCTCCTTTAACACCAGAAAGTATCAATGGGACAGATGAT
GAGAGAACACCCGATGTGACACAGAACTCAGAGCCAAGGGCTGAGCCAGC
TCAGAATGCATTGCCATTTTCACATAGTGCCGGGGATCGAACCCAGGGCC
TCTCTCATGCTAGTTCAGCCATCAGCAAACACTGGGAGGCTGAACTAGCC
ACGCTCAAGGGGAACAATGCCAAGCTCACCGCAGCGCTGCTGGAGTCCAC
TGCCAACGTGAAGCAGTGGAAGCAACAGCTGGCTGCCTACCAGGAGGAGG
CAGAGCGGCTGCACAAGCGGGTCACGGAGCTGGAATGTGTTAGTAGTCAA
GCAAACGCGGTGCACAGCCACAAGACAGAGCTGAGTCAGACAGTGCAGGA
GCTGGAAGAGACCCTAAAAGTAAAGGAAGAGGAAATAGAAAGATTAAAAC
AAGAAATTGATAACGCCAGAGAACTTCAAGAACAGAGGGACTCTTTGACT
CAGAAACTACAGGAAGTTGAGATTCGAAATAAAGACCTGGAGGGGCAGCT
GTCGGAGCTGGAGCAGCGCCTGGAGAAGAGCCAGAGCGAGCAGGACGCTT
TCCGCAGTAACCTGAAGACTCTCCTAGAGATTCTGGACGGGAAAATATTT
GAACTAACAGAATTGCGGGATAATTTGGCCAAGCTACTAGAATGCAGCTA
A.

By “hyper-diverse barcoded plasmid library” is meant a library of plasmids having unique, identifiable barcodes, where the diversity of barcodes, plasmids may be in the hundreds of thousands to millions.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “human synapsin (hSyn promoter)” is meant a nucleic acid molecule, or a fragment thereof, having at least 85% sequence identity to the following nucleotide sequence: AGTGCAAGTGGGTTTTAGGACCAGGATGAGGCGGGGTGGGGGTGCCTACCTGACGACCGACCCCGACCCA CTGGACAAGCACCCAACCCCCATTCCCCAAATTGCGCATCCCCTATCAGAGAGGGGGAGGGGAAACAGGA TGCGGCGAGGCGCGTGCGCACTGCCAGCTTCAGCACCGCGGACAGTGCCTTCGCCCCCGCCTGGCGGCGC GCGCCACCGCCGCCTCAGCACTGAAGGCGCGCTGACGTCACTCGCCGGTCCCCCGCAAACTCCCCTTCCC GGCCACCTTGGTCGCGTCCGCGCCGCCGCCGGCCCAGCCGGACCGCACCACGCGAGGCGCGAGATAGGGG GGCACGGGCGCGACCATCTGCGCTGCGGCGCCGGCGACTCAGCGCTGCCTCAGTCTGCGGTGGGCAGCGG AGGAGTCGTGTCGTGCCTGAGAGCGCAG (SEQ ID NO: 30), wherein the promoter is capable of directing expression of a downstream polynucleotide in a neuron. Exemplary HsYN promoters are described, for example, by Nieuwenhuis et al., Gene Ther 28, 56-74 (2021). Doi: 10.1038/s41434-020-0169-1.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all the nucleic acids delineated herein. In embodiments a ribozyme-assisted circular RNA of the disclosure contains an inhibitory nucleic acid.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “λ bacteriophage antiterminator protein N (λN) peptide” is meant a peptide derived from the N protein of bacteriophage having at least about 85% amino acid sequence identity to the amino acid sequence DAQTRRRERRAEKQAQWKAAN (SEQ ID NO: 31), or a fragment thereof, and capable of RNA binding. In one embodiment, a λN peptide is capable of binding a BoxB polynucleotide. λN peptides are described, for example by Baron-Benhamou et al., Methods in Molecular Biology book series, MIMB volume 257, and by Cilley et al., RNA 3: 57-67, 1997, each of which is incorporated herein by reference in their entirety.

By “λN polynucleotide” is meant a nucleic acid molecule encoding a λN polypeptide. An exemplary λN nucleotide sequence is the following:

(SEQ ID NO: 32)
GACGCACAAACACGACGACGTGAGCGTCGCGCTGAGAAACAAGCTCAATG
GAAAGCTGCAAAC.

By “M9 tag peptide” or “M9 tag” is meant a nuclear export signal peptide, or a fragment thereof, having at least about 85% amino acid sequence identity to the following sequence: NDFGNYNNQSSNFGPMKGGNFGGRSSGPYGGGGQYFAKPRNQGGY (SEQ ID NO: 33),and capable of facilitating export from the cell nucleus of a polypeptide to which the M9 polypeptide is fused.

By “M9 tag polynucleotide” is meant a nucleic acid molecule encoding an M9 tag. An exemplary M9 nucleotide sequence is provided below.

(SEQ ID NO: 34)
AATGATTTTGGCAATTACAACAATCAGTCTTCCAATTTTGGGCCGATGAA
GGGAGGAAACTTTGGAGGCAGGAGCTCTGGCCCTTATGGTGGTGGAGGCC
AGTACTTTGCTAAACCACGGAACCAAGGTGGCTAT.

By “marker” is meant any analyte, protein or polynucleotide having an alteration in expression, level or activity that is associated with a disease or disorder.

By “MS2 coat protein (MS2cp) polypeptide” is meant a polypeptide, or a fragment thereof, having at least about 85% amino acid sequence identity to GenBank Accession No. AGJ84361.1 and capable of binding an MS2 polynucleotide. An exemplary amino acid sequence follows:

(SEQ ID NO: 35)
MASNFTQFVLVDNGGTGDVTVAPSNFANGVAEWISSNSRSQAYKVTCSVR
QSSAQNRKYTIKVEVPKGAWRSYLNMELTIPIFSTNSDCELIVKAMQGLL
KDGNPIPSAIAANSGIY.

By “MS2 coat protein (MS2cp) polynucleotide” is meant a nucleic acid molecule encoding a MS2cp polypeptide. An exemplary MS2cp nucleotide sequence is provided below and at GenBank Accession No. JQ624676.1.

(SEQ ID NO: 36)
ATGGCTTCTAACTTTACTCAGTTCGTTCTCGTCGACAATGGCGGAACTGG
CGACGTGACTGTCGCCCCAAGCAACTTCGCTAACGGGGTCGCTGAATGGA
TCAGCTCTAACTCGCGTTCACAGGCTTACAAAGTAACCTGTAGCGTTCGT
CAGAGCTCTGCGCAGAATCGCAAATACACCATCAAAGTCGAGGTGCCTAA
AGGCGCATGGCGTTCGTACTTAAATATGGAACTAACCATTCCAATTTTCT
CCACGAACTCCGACTGCGAGCTTATTGTTAAGGCAATGCAAGGTCTCCTA
AAAGATGGAAACCCGATTCCCTCAGCAATCGCAGCAAACTCCGGCATCTA
C.

By “MS2 RNA hairpin polynucleotide” is meant a nucleic acid molecule comprising the following sequence: ACATGAGGATCACCCATGT (SEQ ID NO: 37), and variants thereof including 1, 2, 3, 4, 5, or 6 nucleotide alterations capable of being bound by a MS2cp polypeptide.

By “operably linked” refers to a functional linkage between a regulatory sequence and a coding sequence, where a first polynucleotide is positioned adjacent to a second polynucleotide that directs transcription of the first polynucleotide when appropriate molecules are bound to the second polynucleotide. In embodiments the appropriate molecules contain transcriptional activator proteins. The described components are therefore in a relationship permitting them to function in their intended manner. For example, placing a coding sequence under regulatory control of a promoter means positioning the coding sequence such that the expression of the coding sequence is controlled by the promoter.

By “polyadenylation signal sequence” (poly(A) signal sequence) or “poly(A) tail” is meant a sequence of multiple adenosine monophosphates at the 3′-end of mRNA or cDNA. The poly(A) tail is particularly important for nuclear export, translation, and for stabilizing or protecting mRNA from nucleases.

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “positioned for expression” is meant that a polynucleotide is positioned adjacent to a DNA sequence that directs transcription or translation of the sequence.

By “PP7 coat protein (PP7cp) polypeptide” is meant a polypeptide, or fragments thereof, having at least about 85% amino acid sequence identity to NCBI Ref. Seq. Accession No. NP_042305.1 and capable of binding a PP7 polynucleotide. An exemplary amino acid sequence follows:

(SEQ ID NO: 38)
MSKTIVLSVGEATRTLTEIQSTADRQIFEEKVGPLVGRLRLTASLRQNGA
KTAYRVNLKLDQADVVDCSTSVCGELPKVRYTQVWSHDVTIVANSTEASR
KSLYDLTKSLVATSQVEDLVVNLVPLGR.

By “PP7 coat protein (PP7cp) polynucleotide” is meant a nucleic acid molecule encoding a PP7cp polypeptide. An exemplary PP7cp nucleotide sequence is provided below and at NCBI Ref. Seq. Accession No. NC_001628.1.

(SEQ ID NO: 39)
TCCAAAACCATCGTTCTTTCGGTCGGCGAGGCTACTCGCACTCTGACTGA
GATCCAGTCCACCGCAGACCGTCAGATCTTCGAAGAGAAGGTCGGGCCTC
TGGTGGGTCGGCTGCGCCTCACGGCTTCGCTCCGTCAAAACGGAGCCAAG
ACCGCGTATCGCGTCAACCTAAAACTGGATCAGGCGGACGTCGTTGATTG
CTCCACCAGCGTCTGCGGCGAGCTTCCGAAAGTGCGCTACACTCAGGTAT
GGTCGCACGACGTGACAATCGTTGCGAATAGCACCGAGGCCTCGCGCAAA
TCGTTGTACGATTTGACCAAGTCCCTCGTCGCGACCTCGCAGGTCGAAGA
TCTTGTCGTCAACCTTGTGCCGCTGGGCCGT.

By “PP7 polynucleotide” is meant a nucleic acid molecule comprising a sequence selected from GGAGCAGACGATATGGCGTCGCTCC (SEQ ID NO: 40), CCAGCAGAGCATATGGGCTCGCTGG (SEQ ID NO: 41), and variants thereof including 1, 2, 3, 4, 5, or 6, nucleotide alterations and capable of being bound by a PP7cp polypeptide.

By “retrograde infection” is meant spread of a virus from an axon terminal to a parent neuron, where the direction of retrograde spread of a virus is opposite to that of a nerve impulse. A non-limiting example of a viral vector capable of retrograde infection of a cell is a retrograde adeno-associated virus (retroAAV) vector.

By “ribozyme” is meant an RNA sequence that hybridizes to a complementary sequence in a substrate RNA and cleaves the substrate RNA in a sequence specific manner at a substrate cleavage site. Typically, a ribozyme contains a catalytic region flanked by two binding regions. The ribozyme binding regions hybridize to the substrate RNA, while the catalytic region cleaves the substrate RNA at a substrate cleavage site to yield a cleaved RNA product. The nucleotide sequence of the ribozyme binding regions may be completely complementary or partially complementary to the substrate RNA sequence with which the ribozyme hybridizes.

By “RNA-binding protein” is meant a protein capable of binding an RNA molecule. In embodiments, an RNA-binding protein binds a hairpin structure formed by an RNA molecule. Non-limiting examples of RNA-binding proteins include PP7cp, tdPP7cp, MS2cp, tdMS2cp, and λN.

As used herein, “obtaining” as in “obtaining an agent” includes synthesizing, purchasing, or otherwise acquiring the agent.

By “postsynaptic density 95 Fibronectin Intrabodies Generated with mRNA Display (PSD95.FingR) polypeptide” is meant a polypeptide, or fragments thereof, having at least about 85% amino acid sequence identity to the following sequence: MLEVKEASPTSIQISWVLHLRHVRYYRITYGETGGNSPVQEFTVPGSKSTATISGLKPGVDYTI TVYAVTIFSAYRSAWPPISINYRTGT (SEQ ID NO: 42), and capable of facilitating localization of a protein to which the PSD95.FingR polypeptide is fused.

By “postsynaptic density 95 Fibronectin Intrabodies Generated with mRNA Display (PSD95.FingR) polynucleotide” is meant a nucleic acid molecule encoding a PSD95.FingR polypeptide. An exemplary PSD95.FingR nucleotide sequence is provided below.

(SEQ ID NO: 43)
ATGCTCGAAGTCAAGGAAGCATCACCAACCAGCATCCAGATCAGCTGGGT
GCTCCACTTGCGCCACGTTCGCTACTACCGCATCACCTACGGTGAAACTG
GTGGCAATAGCCCTGTCCAGGAATTCACCGTGCCTGGCAGCAAGTCCACT
GCTACCATCAGCGGCCTGAAACCTGGTGTCGACTATACCATCACGGTGTA
CGCCGTCACGATCTTCAGCGCCTACCGCTCCGCCTGGCCGCCGATCTCCA
TCAACTACCGCACCGGAACC.

By “reduces” is meant a negative alteration of at least 10%, 25%, 50%, 75%, or 100%.

By “reference” is meant a standard or control condition. In embodiments, a reference is a cell (e.g., a neuron) or tissue (e.g., brain tissue) not contacted with a vector or polynucleotide of the present disclosure. In some cases, a reference is a healthy cell or subject. Further non-limiting examples of references include a cell or tissue prior to being contacted with a vector or polynucleotide of the present disclosure, a first polynucleotide or vector including an additional element (e.g., an RNA hairpin or polynucleotide-encoding sequence) or lacking an element relative to a second polynucleotide or vector, a viral vector with a previously-characterized tropism, or a linear RNA molecule.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “RNA 2′,3′-cyclic phosphate and 5′-OH ligase (RtcB) polypeptide” is meant a polypeptide, or fragments thereof, having at least about 85% amino acid sequence identity to NCBI Ref. Seq. Accession No. WP_001105504.1 and capable of catalyzing the ligation of two RNA molecules to each other. An exemplary amino acid sequence follows:

(SEQ ID NO: 44)
MNYELLTTENAPVKMWTKGVPVEADARQQLINTAKMPFIFKHIAVMPDV
HLGKGSTIGSVIPTKGAIIPAAVGVDIGCGMNALRTALTAEDLPENLAE
LRQAIETAVPHGRTTGRCKRDKGAWENPPVNVDAKWAELEAGYQWLTQK
YPRFLNTNNYKHLGTLGTGNHFIEICLDESDQVWIMLHSGSRGIGNAIG
TYFIDLAQKEMQETLETLPSRDLAYFMEGTEYFDDYLKAVAWAQLFASL
NRDAMMENVVTALQSITQKTVRQPQTLAMEEINCHHNYVQKEQHFGEEI
YVTRKGAVSARAGQYGIIPGSMGAKSFIVRGLGNEESFCSCSHGAGRVM
SRTKAKKLFSVEDQIRATAHVECRKDAEVIDEIPMAYKDIDAVMAAQSD
LVEVIYTLRQVVCVKG.

By “RNA 2′,3′-cyclic phosphate and 5′-OH ligase (RtcB) polynucleotide” is meant a nucleic acid molecule encoding a RTcB polypeptide. An exemplary RtcB nucleotide sequence is provided below.

(SEQ ID NO: 45)
AACTATGAGCTTTTGACCACTGAGAACGCTCCTGTTAAGATGTGGACAA
AAGGCGTGCCTGTAGAGGCCGACGCTCGGCAGCAACTCATTAACACCGC
CAAGATGCCCTTTATTTTCAAGCATATTGCCGTGATGCCTGATGTCCAT
CTTGGTAAGGGTTCAACAATCGGGAGCGTCATCCCTACCAAGGGTGCCA
TCATTCCAGCCGCCGTAGGAGTAGATATTGGATGCGGCATGAACGCACT
TAGAACAGCTCTGACCGCCGAGGATCTTCCCGAGAACCTCGCTGAACTG
CGACAGGCAATCGAGACAGCAGTTCCTCACGGCAGAACCACAGGCAGGT
GTAAGAGAGATAAGGGCGCATGGGAAAACCCCCCCGTGAATGTCGACGC
AAAATGGGCAGAGTTGGAAGCTGGGTATCAATGGCTGACCCAAAAGTAC
CCACGGTTCCTCAATACTAATAACTATAAGCACCTTGGGACACTCGGAA
CCGGCAACCACTTCATAGAAATATGCCTGGACGAGTCAGATCAAGTTTG
GATAATGCTCCACTCTGGTTCACGGGGCATTGGCAACGCTATAGGAACA
TACTTTATAGACCTGGCCCAGAAAGAGATGCAAGAAACATTGGAAACTC
TCCCAAGTAGGGACCTCGCTTACTTCATGGAGGGAACTGAGTATTTCGA
TGATTATCTGAAAGCCGTAGCATGGGCACAGTTGTTCGCCTCCTTGAAT
AGGGATGCAATGATGGAGAATGTCGTCACTGCTCTTCAAAGTATCACCC
AAAAAACAGTACGCCAACCTCAGACTCTGGCAATGGAAGAGATCAACTG
TCATCATAACTACGTACAAAAGGAACAACACTTCGGCGAAGAGATCTAT
GTTACCCGGAAAGGGGCCGTCTCAGCTAGGGCAGGCCAATACGGCATAA
TCCCTGGCTCTATGGGTGCAAAAAGCTTCATAGTTCGAGGCCTTGGGAA
CGAGGAGAGCTTTTGTAGCTGTAGCCACGGGGCTGGTCGGGTGATGTCC
CGGACTAAAGCTAAAAAATTGTTCTCTGTTGAGGACCAAATACGGGCTA
CCGCACACGTAGAATGCCGGAAGGACGCCGAGGTCATCGACGAAATCCC
TATGGCCTACAAGGACATTGACGCAGTTATGGCCGCACAGTCTGACCTG
GTGGAAGTTATATATACACTGAGGCAAGTAGTATGTGTGAAGGGA.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. Non-limiting examples of animals include a human or non-human mammal, such as a bovine, equine, canine, ovine, rodent, or feline.

By “synaptophysin (SYP1; SYPH) polypeptide” is meant a polypeptide, or fragment thereof, having at least about 85% amino acid sequence identity to NCBI Ref. Seq. Accession No. NP_036796.1, which is provided below, and capable of mediating localization of a polypeptide to a pre-synapse compartment of a cell. SYP1 is described in Lin, J., et al., Neuron., 79:241-253, the disclosure of which is incorporated herein by reference in its entirety for all purposes.
NP 036796.1 synaptophysin [Rattus norvegicus]

(SEQ ID NO: 46)
MDVVNQLVAGGQFRVVKEPLGFVKVLQWVFAIFAFATCGSYTGELRLSVE
CANKTESALNIEVEFEYPFRLHQVYFDAPSCVKGGTTKIFLVGDYSSSAE
FFVTVAVFAFLYSMGALATYIFLQNKYRENNKGPMMDFLATAVFAFMWLV
SSSAWAKGLSDVKMATDPENIIKEMPMCRQTGNTCKELRDPVTSGLNTSV
VFGFLNLVLWVGNLWFVFKETGWAAPFMRAPPGAPEKQPAPGDAYGDAGY
GQGPGGYGPQDSYGPQGGYQPDYGQPASGGGGYGPQGDYGQQGYGQQGAP
TSFSNQM.

By “synaptophysin (SYP1; SYPH) polynucleotide” is meant a nucleic acid molecule encoding a SYP1 polypeptide. An exemplary SYP1 nucleotide sequence is provided below and at NCBI. Ref. Seq. Accession No. NM_012664.3.
NM_012664.3:16-939 Rattus norvegicus synaptophysin (Syp), mRNA

(SEQ ID NO: 47)
ATGGACGTGGTGAATCAGCTGGTGGCTGGGGGTCAGTTCCGGGTGGTCAA
GGAGCCCCTTGGCTTCGTGAAGGTGCTGCAGTGGGTCTTTGCCATCTTCG
CCTTTGCTACGTGTGGCAGCTACACCGGGGAGCTTCGGCTGAGCGTGGAG
TGTGCCAACAAGACGGAGAGTGCCCTCAACATCGAAGTTGAATTCGAGTA
CCCCTTCAGGCTGCACCAAGTGTACTTTGATGCACCCTCCTGCGTCAAAG
GGGGCACTACCAAGATCTTCCTGGTTGGGGACTACTCCTCGTCGGCTGAA
TTCTTTGTCACCGTGGCTGTGTTTGCCTTCCTCTACTCCATGGGGGCCCT
GGCCACCTACATCTTCCTGCAGAACAAGTACCGAGAGAACAACAAAGGGC
CTATGATGGACTTTCTGGCTACAGCCGTGTTCGCTTTCATGTGGCTAGTT
AGTTCATCAGCCTGGGCCAAAGGCCTGTCCGATGTGAAGATGGCCACGGA
CCCAGAGAACATTATCAAGGAGATGCCCATGTGCCGCCAGACAGGGAACA
CATGCAAGGAACTGAGGGACCCTGTGACTTCAGGACTCAACACCTCAGTG
GTGTTTGGCTTCCTGAACCTGGTGCTCTGGGTTGGCAACTTATGGTTCGT
GTTCAAGGAGACAGGCTGGGCAGCCCCATTCATGCGCGCACCTCCAGGCG
CCCCGGAAAAGCAACCAGCACCTGGCGATGCCTACGGCGATGCGGGCTAC
GGGCAGGGCCCCGGAGGCTATGGGCCCCAGGACTCCTACGGGCCTCAGGG
TGGTTATCAACCCGATTACGGGCAGCCAGCCAGCGGTGGCGGTGGCTACG
GGCCTCAGGGCGACTATGGGCAGCAAGGCTATGGCCAACAGGGTGCGCCC
ACCTCCTTCTCCAATCAGATGTAA.

Ranges provided herein are understood to be shorthand for all the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

By “U6 promoter” is meant a nucleic acid molecule, or fragments thereof, having at least 85% sequence identity to the following nucleotide sequence and capable of facilitating transcription from a downstream polynucleotide sequence:

(SEQ ID NO: 48)
GAGGGCCTATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGAC

By “unique molecular identifier” or “UMI” is meant a short nucleic acid sequence that is identifiable. UMIs are useful, for example, in high-throughput sequencing techniques, such as but not limited to, single-cell RNA-seq. The UMIs may be used to not only detect, but also to quantify. In embodiments of the disclosure, the UMIs are not viral barcodes.

By “vesicle-associated membrane protein 2A (VAMP2A) polypeptide” is meant a polypeptide, or fragments thereof, with at least about 85% amino acid sequence identity GenBank Accession No. AAA60604.1, and capable of facilitating localization of a protein to which the VAMP2A polypeptide is fused to a pre-synapse compartment of a cell. An exemplary amino acid sequence follows:

(SEQ ID NO: 49)
MSATAATVPPAAPAGEGGPPAPPPNLTSNRRLQQTQAQVDEVVDIMRVNV
DKVLERDQKLSELDDRADALQAGASQFETSAAKLKRKYWWKNLKMMIILG
VICAIILIIIIVYFST.

By “vesicle-associated membrane protein 2A (VAMP2A) polynucleotide” is meant a nucleic acid molecule encoding a VAMP2A polypeptide. An exemplary VAMP2A nucleotide sequence is provided below and at GenBank Accession No. AH002993.2.

(SEQ ID NO: 50)
ATGTCGGCTACCGCTGCCACCGTCCCGCCTGCCGCCCCGGCCGGCGAGGG
TGGCCCCCCTGCACCTCCTCCAAACCTTACTAGTAACAGGAGACTGCAGC
AGACCCAGGCCCAGGTGGATGAGGTGGTGGACATCATGAGGGTGAATGTG
GACAAGGTCCTGGAGCGGGACCAGAAGTTGTCGGAGCTGGATGACCGTGC
AGATGCCCTCCAGGCAGGGGCCTCCCAGTTTGAAACAAGTGCAGCCAAGC
TCAAGCGCAAATACTGGTGGAAAAACCTCAAGATGATGATCATCTTGGGA
GTGATCTGCGCCATCATCCTCATCATCATCATCGTTTACTTCAGCACT.

By “vector” is meant a nucleic acid molecule, for example, a plasmid, cosmid, virus, or bacteriophage that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers. In one embodiment, the vector is a plasmid. Suitable viral expression vectors include, but are not limited to, viral vectors based on vaccinia virus; poliovirus; adenovirus (see, e.g., PCT Publication Nos. WO 94/12649 to Gregory et al., WO 93/03769 to Crystal et al., WO 93/19191 to Haddada et al., WO 94/28938 to Wilson et al., WO 95/11984 to Gregory, and WO 95/00655 to Graham, which are hereby incorporated by reference in their entirety); adeno-associated virus (see, e.g., Ali et al., Hum. Gene Ther. 9:8186 (1998), Flannery et al., PNAS 94:6916-6921 (1997); Bennett et al., Invest. Opthalmol. Vis. Sci. 38:2857-2863 (1997); Jomary et al., Gene Ther. 4:683-690 (1997), Rolling et al., Hum. Gene Ther. 10:641-648 (1999); Ali et al., Hum. Mol. Genet. 5:591-594 (1996); Samulski et al., J. Vir. 63:3822-3828 (1989); Mendelson et al., Virol. 166:154-165 (1988); and Flotte et al., PNAS 90:10613-10617 (1993), which are hereby incorporated by reference in their entirety); SV40; herpes simplex virus; human immunodeficiency virus (see, e.g., Miyoshi et al., PNAS 94:10319-23 (1997); Takahashi et al., J. Virol. 73:781-7816 (1999), which are hereby incorporated by reference in their entirety); a retroviral vector, e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus and the like.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The following abbreviations of tissue regions are used in the present disclosure and are based on the Allen Mouse Brain Reference Atlas. Tissue region abbreviations: CTX, cerebral cortex; HPF, hippocampal formation; STR, striatum; TH, thalamus; RSP, retrosplenial cortex; L2/3, layer 2/3; L4, layer 4; L5, layer 5; L6, layer 6; FC, Fasciola cinerea; DG, dentate gyrus; so, stratum oriens; sp, pyramidal layer; sr, stratum radiatum; slm, stratum lacunosum-moleculare; mo, molecular layer; sg, granule cell layer; po, polymorph layer; CP, caudoputamen; RT, reticular nucleus of the thalamus; MH, medial habenula; LH, lateral habenula; v3, third ventricle; VL, lateral ventricle; cing, cingulum bundle; df, dorsal fornix; cc, corpus callosum; alv, alveus; fi, fimbria; int, internal capsule; MOBgr, main olfactory bulb, granule layer; AOBgr, accessory olfactory bulb; OBmi, olfactory bulb, mitral layer; OBopl, olfactory bulb, outer plexiform layer; OBgl, olfactory bulb, glomerular layer; Llm, cerebral cortical layer 1, medial part; HPFslm/sr/so, hippocampal formation stratum lacunosum-moleculare/stratum radiatum/stratum oriens; L1l, cerebral cortical layer 1, lateral part; PRE, presubiculum; POST, postsubiculum; PL, prelimbic area; ACA, anterior cingulate area; AI, agranular insular area; CLA, claustrum; EP, endopiriform nucleus; AONm, anterior olfactory nucleus, medial part; TTv, taenia tecta, ventral part; ILA, infralimbic area; ENTI, entorhinal area, lateral part; ENTm, entorhinal area, medial part; SUBsp, subiculum, pyramidal layer; COAp, cortical amygdalar area, posterior part; PA, posterior amygdalar nucleus; LA, lateral amygdalar nucleus; DGd-sg, dentate gyrus, dorsal part, granule cell layer; DGv-sg, dentate gyrus, ventral part, granule cell layer; DGmo/po, dentate gyrus, molecular layer/polymorph layer; CAlsp, field CAT, pyramidal layer; CA2sp, field CA2, pyramidal layer; IG, indusium griseum; CA3sp, field CA3, pyramidal layer; CBXmo, cerebellar cortex, molecular layer; CBXd-gr, cerebellar cortex, dorsal part, granular layer; CBXv-gr, cerebellar cortex, ventral part, granular layer; CBXpu, cerebellar cortex, Purkinje layer; THI, lateral TH; THam, anterior-medial TH; THpm, posterior medial TH; RE, nucleus of reuniens; MHv, medial habenula, ventral part; MHd, medial habenula, dorsal part; STRd-al, dorsal striatum, anterior-lateral enriched; STRd-pm, dorsal striatum, posterior-medial enriched; STRv-al, ventral striatum, anterior-lateral enriched; STR-periV, periventricular area of striatum; STRv-pm, ventral striatum, posterior-medial enriched; CEAl, central amygdalar nucleus, lateral part; STRv-OT, ventral striatum, olfactory tubercle; STRv-isl, ventral striatum, islands of Calleja; LS, lateral septal nucleus; PALv, pallidum, ventral region; PALm, pallidum, medial region; TRS, triangular nucleus of septum; MEA, medial amygdalar nucleus; BMA, basomedial amygdalar nucleus; COAa, cortical amygdalar area, anterior part; IA, intercalated amygdalar nucleus; SEZ, subependymal zone; SFO, subfornical organ; HYam, hypothalamus, anterior medial enriched; LHA, lateral hypothalamic area; TM, tuberomammillary nucleus; VMH, ventromedial hypothalamic nucleus; DMH, dorsomedial nucleus of the hypothalamus; PeF, perifornical nucleus; ARH, arcuate hypothalamic nucleus; PM, premammillary nucleus; MM, medial mammillary nucleus; PVH, paraventricular hypothalamic nucleus; SCH, suprachiasmatic nucleus. PAGd, periaqueductal gray, dorsal part enriched; HYpm, hypothalamus, posterior-medial part enriched; HYal, hypothalamus, anterior-lateral enriched; SC, superior colliculus; PCG, pontine central gray; IC, inferior colliculus; EW, Edinger-Westphal nucleus; PALd, pallidum, dorsal region; ZI, zona incerta; P, pons; MYa, medulla, anterior enriched; MYp, medulla, posterior enriched; PSV, principal sensory nucleus of the trigeminal; SPVC, spinal nucleus of the trigeminal, caudal part; STN, subthalamus nucleus; SNr, substantia nigra, reticular part; MV, medial vestibular nucleus; Pm, pons, medial part; MYm, medulla, medial enriched; IO, inferior olivary complex; MYd, medulla, dorsal part; VTA, ventral tegmental area; SNc, substantia nigra, compact part; RR, midbrain reticular nucleus, retrorubral area; IPN, interpeduncular nucleus; LC, locus coeruleus; VII, Facial motor nucleus; V, motor nucleus of trigeminal; III, oculomotor nucleus; PPN, pedunculopontine nucleus; NTS, nucleus of the solitary tract; PAGpv, periaqueductal gray, posterior ventral part; DR, dorsal nucleus raphe; FB, forebrain; HB, hindbrain; sptV, spinal tract of the trigeminal nerve; sctv, ventral spinocerebellar tract; onl, olfactory nerve layer of main olfactory bulb; VW, ventricular wall; chpl, choroid plexus; SCO, subcommissural organ; MNG, meninges; MO, somatomotor areas; MOp, primary MO; SS, somatosensory area; SSp, primary SS; SSs, secondary SS; VISC, visceral area; Alp, agranular insular area, posterior part; sAMY, striatum-like amygdalar nuclei; VIS, visual area; AUD, auditory area; TEa, temporal association area; CTXsp, cortical subplate; AQ, cerebral aqueduct.

### BRIEF DESCRIPTION OF THE DRAWINGS

FIGS. 1A-1D provide schematics showing a collection of RNA elements that facilitate nuclear export and their secondary structures. FIG. 1A provides a schematic showing Rev response elements (RRE), which enable the nuclear export of intron-containing HIV RNA. Figure discloses SEQ ID NO: 196. FIG. 1B provides a schematic showing the adenovirus VA1 RNA, which contains a consensus terminal mini helical structure that facilitates nuclear export (Gwizdek C, et al., “Terminal minihelix, a novel RNA motif that directs polymerase III transcripts to the cell cytoplasm. Terminal minihelix and RNA export.” J Biol Chem 276: 25910-25918 (2001)). FIG. 1C shows constitutive transcript element (CTE), a two-fold symmetrical element from Mason-Pfizer Monkey Virus (MPMV), and one symmetrical half of the CTE (hCTE). Figure discloses SEQ ID NOS 197 and 198. FIG. 1D provides a schematic of BC1, a rodent neuron-specific ncRNA localized in the cytoplasm. Figure discloses SEQ ID NO: 141.

FIGS. 2A-2D provide a schematic and gel images relating to circular RNA expression vectors and their validation in vitro. FIG. 2A shows schemes of barcode circular RNA expression system (see, e.g., U.S. 2021/034052 A1, the disclosure of which is incorporated herein by reference in its entirety for all purposes). Ribozyme-assisted circular RNAs (racRNAs) can be expressed from a human U6 promoter to produce circular RNAs with a PP7 hairpin and a barcode region (racPP7). FIGS. 2B-2C show illustrations of racRNAs inserted with the hCTE or BC1 RNA hairpin. FIG. 2D shows in vitro validation of circular RNA formation. In vitro transcribed circular RNA was treated with RNA ligase RtcB and then RNase R. After RtcB ligation, a band resistant to RNase R was formed (marked by the arrows), representing circular RNA species. M, RNA markers.

FIG. 3 shows endogenous export adaptor or receptor proteins for various defined RNA structures. Key export mediators for each of the categories of RNAs are highlighted.

FIG. 4 provides a schematic showing potential mechanisms of how nuclear-cytoplasmic shuttling RNA binding proteins facilitate the nuclear export of its RNA partner. The M9 tag from heterogeneous nuclear ribonucleoproteins enables the shuttling of the fusion protein. An additional nuclear export signal (NES) is included to enhance export.

FIGS. 5A-5G show validation of RNA barcode nuclear export strategies in Neuro-2A cells. FIG. 5A shows schematics showing racRNA carrying PP7 hairpin and RNA barcode sequences, and protein partners for membrane anchoring and nuclear exporting.

FIGS. 5B-5G show STARmapping of the indicated barcode racRNAs 24 hours after transfection with racRNA expression plasmids. Left, plasmids named by their composed transgene elements; middle, raw fluorescent images of racRNA barcode (STARmap), protein partners (immunostaining of epitope tags), nuclei (DAPI), and merged channels; right, fluorescent signal intensity profiles across the white dashed lines indicated in the merged-channel images. Scale bar, 20 μm. In FIGS. 5B-5G, a description of the vector administered to the cells is provided to the left of each figure, where the first term of the description (i.e., “pAAV”) indicates that the vector was an adeno-associated virus vector containing a polynucleotide encoding from 5′ to 3′ the components listed following the term “pAAV.” In FIGS. 5B-5G “pAAV” indicates an AAV vector; “U6” and “hSyn” indicate promoters; “racRNA” indicates a nucleotide sequence encoding a “ribozyme-assisted circular RNA”; “PP7” and “hCTE” indicate RNA hairpins; “FLAG” and “V5” indicate epitope tags; “PP7cp” indicates the RNA-binding domain PP7 coat protein; “Far” indicates a farnseylation motif; “linear” indicates a non-circular RNA molecule; “3λNLS” indicates three tandem repeats of a nuclear localization signal; “RtcB” indicates an RNA ligase; T2A indicates a self-leaving peptide; and DDX39A indicates an RNA nuclear transport protein. The shaded regions of the plots of FIGS. 5B-5G represent the nucleus of the cell.

FIGS. 6A-6C show combining cis- and trans-RNA exporting elements in proliferating cell cultures. FIG. 6A shows schematics showing designs of racRNA with cis-elements facilitating RNA export and trans protein partners for membrane anchoring and nuclear exporting, respectively. FIGS. 6B-6C show STARmapping of the barcode racRNAs 24 hours after transfection with racRNA expression plasmids in HeLa cell (FIG. 6B) and Neuro-2A cells (FIG. 6C). Left, plasmids named by their composed transgene elements; middle, raw fluorescent images of racRNA barcode (STARmap), protein partners (immunostaining of epitope tags), nuclei (DAPI), and merged channels; right, fluorescent signal intensity profiles across the white dashed lines indicated in the merged-channel images. Scale bar, 20 μm. In FIGS. 6B and 6C, a description of the vector administered to the cells is provided to the left of each figure, where the first term of the description (i.e., “pAAV”) indicates that the vector was an adeno-associated virus vector containing a polynucleotide encoding from 5′ to 3′ the components listed following the term “pAAV.” In FIGS. 6B and 6C “pAAV” indicates an AAV vector; “U6” and “CAG” indicate promoters; “rac” indicates a nucleotide sequence encoding a “ribozyme-assisted circular RNA”; “PP7” and “hCTE” indicate RNA hairpins; “M9” indicates an M9 tag; “NES” indicates a nuclear export signal; “FLAG” and “V5” indicate epitope tags; “PP7cp” indicates the RNA-binding domain PP7 coat protein; “Far” indicates a famseylation motif; T2A indicates a self-leaving peptide. The shaded regions of the plots of FIGS. 6B and 6C represent the nucleus of the cell.

FIGS. 7A-7C show cis- and trans-RNA exporting element screening in primary rat cortical neurons. FIG. 7A is schematics showing designs of racRNA with cis-elements facilitating RNA export and trans protein partners for membrane anchoring and nuclear exporting, respectively. FIGS. 7B and 7C show STARmapping of barcode RNAs 7 days after electroporation into primary neurons. Left, plasmids named by their composed transgene elements; right, raw fluorescent images of racRNA barcode (STARmap), protein partners (immunostaining of epitope tags), nuclei (DAPI), and merged channels. Scale bar, 50 μm. In FIGS. 7B and 7C, a description of the vector administered to the cells is provided to the left of each figure, where the first term of the description (i.e., “pAAV”) indicates that the vector was an adeno-associated virus vector containing a polynucleotide encoding from 5′ to 3′ the components listed following the term “pAAV.” In FIGS. 7B and 7C “pAAV” indicates an AAV vector; “U6” and “hSyn” indicate promoters; “rac” indicates a nucleotide sequence encoding a “ribozyme-assisted circular RNA”; “PP7,” “hCTE,” “BC1,” and “BC70,” indicate RNA hairpins; “M9” indicates an M9 tag; “NES” indicates a nuclear export signal; “mCherry” indicates a fluorescent protein; “FLAG” and “V5” indicate epitope tags; “PP7cp” indicates the RNA-binding domain PP7 coat protein; “RtcB” indicates an RNA ligase; “DDX39A” indicates an RNA nuclear transport protein; “3XNLS” indicates three tandem repeats of a nuclear localization signal; “Far” indicates a farnseylation motif; T2A indicates a self-leaving peptide. The shaded regions of the plots of FIGS. 7B and 7C represent the nucleus of the cell.

FIGS. 8A-8G show combining cis- and trans-RNA exporting elements in primary rat cortical neurons. FIG. 8A is schematics showing designs of racRNA with cis-elements facilitating RNA export and trans protein partners for membrane anchoring and nuclear exporting, respectively. FIGS. 8B-8G show STARmapping of barcode RNAs 14 days after electroporation into primary neurons. Left, plasmids named by their composed transgene elements; right, raw fluorescent images of racRNA barcode (STARmap), protein partners (immunostaining of epitope tags) (FIGS. 8B-8D) or linear RNAs (STARmap) (FIGS. 8E-8G), nuclei (DAPI), and merged channels. Scale bar, 50 pm. FIGS. 8B-8G, a description of the vector administered to the cells is provided to the left of each figure, where the first term of the description (i.e., “pAAV”) indicates that the vector was an adeno-associated virus vector containing a polynucleotide encoding from 5′ to 3′ the components listed following the term “pAAV.” In FIGS. 8B-8G “pAAV” indicates an AAV vector; “U6” and “TRE” indicate promoters, where expression from the “TRE” promoter is activated when cells are contacted with a transducer; “rac” indicates a nucleotide sequence encoding a “ribozyme-assisted circular RNA”; “PP7” and “hCTE” indicate RNA hairpins; “M9” indicates an M9 tag; “NES” indicates a nuclear export signal; “FLAG” and “V5” indicate epitope tags; “mCherry” indicates a fluorescent protein; “PP7cp” indicates the RNA-binding domain PP7 coat protein; “30A” indicates a chain of three As; “Far” indicates a farnseylation motif; “w/o transducer” and “w/transducer” indicate cells grown in the absence (i.e., without) or presence (i.e. with) of a transducer; T2A indicates a self-leaving peptide. The shaded regions of the plots of FIGS. 8B-8G represent the nucleus of the cell.

FIGS. 9A-9E show synaptic targeting constructs. FIGS. 9A-9D are schematics showing construct designs for targeting pre-synapse/axons (FIG. 9A), excitatory post-synapse (FIG. 9B), inhibitory post-synapse (FIG. 9C), and dendrites (FIG. 9D). Different RNA barcode sequences, and orthogonal pairs of RNA hairpins and epitope-tagged RNA hairpin binding proteins were assigned to individual categories of plasmids to characterize multiple constructs in the same cell. FIG. 9E shows STARmapping of racRNA barcodes in primary rat cortical neurons co-electroporated with pre- and post-synaptic targeting plasmids. Neuronal axons and dendrites were preferentially stained with anti-TAU and anti-MAP2 antibodies. Size of the field of view, 460 μm. In FIGS. 9A-9E, “M9” indicates an M9 tag; “NES” indicates a nuclear export signal; “FLAG,” “V5,” and “HA” indicate epitope tags; “tdPP7cp,” “PP7cp,” “MS2cp,” “tdMS2cp,” and “λN” indicate the RNA-binding domains; “hSyn” indicates a promoter; and T2A indicates a self-leaving peptide. The terms CCR5TC, KRAB, IL2RGTC, PSD95.FingR, and GPHN.FingR and their roles in gene regulation are described in Bensussen, et al. “A Viral Toolbox of Genetically Encoded Fluorescent Synaptic Tags,” iScience, 23:101330 (2020), the disclosure of which is incorporated herein by reference in its entirety for all purposes.

FIGS. 10A-10D show validating RNA barcode export strategies in vivo in the adult mouse brain. FIG. 10A shows schematics of the transfer plasmids used for AAV-PHP.eB mix packaging. Different RNA barcode sequences, and orthogonal pairs of RNA hairpins and epitope-tagged RNA hairpin binding proteins were assigned to individual categories of plasmids to characterize multiple constructs in the same cell. FIG. 10B shows representative CA3 projection images from the Allen Mouse Brain Connectivity Database. EGFP-expression anterograde AAV was injected into the CA3 of the wild-type mice, and brain slices were imaged by two-photon microscopy. FIG. 10C shows STARmapping of RNA barcodes of four different export designs in thin mouse brain slices two weeks after stereotactic injection of AAV into the hippocampal CA3 region, shown as fluorescent images of the maximum projection of a 10-μm z-stack. Right panels show zoom-in views of individual fluorescent channels of the region highlighted in the square on the left. FIG. 10D shows STARmapping of RNA barcodes of four different export designs in thick mouse brain slices after three weeks of AAV expression. Top right, x-y, y-z, and x-z views of the hippocampal region highlighted in the rectangle on the left; bottom, 3D views of the CA3/DG region highlighted in the square in the top-right panel. The terms used in FIGS. 10A-10D are described above for FIGS. 5A-9E.

FIG. 11 provides a schematic overview of a proof of concept of RNA barcode-assisted morphology tracing in primary neuronal cultures. Images (a) and (b) of FIG. 11 shows STARmapping of RNA barcodes of four different export designs (a) and immunofluorescent staining of MAP2 and Flag-tagged proteins (b) in neuronal cultures two weeks after electroporation. Each plasmid was electroporated into separate neuron populations and then co-cultured. The merged image of fluorescent channels with DAPI (nucleus) was shown as the maximum projection of a 10-μm z-stack. Image (c) of FIG. 11 shows zoom-in view of the rectangle highlighted in image (a) of FIG. 11. Image (d) of FIG. 11 shows RNA barcode spot identified in Image (c) of FIG. 11. Each dot (with transparency) represents an RNA barcode molecule. Image (e) of FIG. 11 shows a neuron identified by ClusterMap based on RNA barcode identities and local RNA barcode densities in image (d) of FIG. 11. Image (f) of FIG. 11 shows zoom-in view of the rectangle highlighted in Image G of FIG. 11 showing the Anti-Flag fluorescent channel. Image G of FIG. 11 shows overlaid images of the RNA-barcode-identified cell (Image (e) of FIG. 11) over the ground-truth membrane-tethered Flag proteins (Image (f) of FIG. 11). The terms used in FIG. 11 are described above for FIGS. 5A-9E.

FIGS. 12A-12E show AAV-PHP.eB tropism profiling in the adult mouse brain. FIG. 12A shows schematics of AAV.PHP.eB tropism characterization across adult mouse brain. Profiling molecular cell types and barcoded AAV in the same biological sample enables systematic AAV tropism characterization. FIG. 12B shows STARmap PLUS was performed to detect single RNA molecules of both a targeted list of 1,022 endogenous genes and trans-expressed barcodes. The mRNA spot matrix was converted to a cell-by-gene expression matrix via ClusterMap. FIG. 12C shows circular RNA expression on representative coronal slices. Each dot represents a cell color-coded by its barcode expression level. FIG. 12D shows raw fluorescent images of STARmap PLUS SEDAL sequencing of a representative brain slice. Left panels show the image stack maximum projection of SEDAL sequencing cycles 1 and 7, merged into an entire half slice. The top right panels show zoomed-in views of SEDAL seq cycles 1 to 7 and amplicons colored by gene identity from the square highlighted in the left panels. The bottom-right panels show zoomed-in views of the square highlighted in the top right panels. FIG. 12E shows boxplots of circular RNA expression levels across molecular cell types in sagittal and coronal slices, respectively. Boxplot elements: vertical line, median; box, first quartile to the third quartile; whiskers, 2.5-97.5%. Numbers in parentheses, number of cells in the group.

FIGS. 13A-13C show Projection pattern decoding at single-neuron resolution by applying racRNA barcode system. FIG. 13A shows schematics of single-neuron projection pattern mapping in a certain brain region. AAVretro encoding different barcodes are intracranially injected into different downstream brain regions of a certain brain region, e.g., mPFC, which is dissected after AAV retrograde labeling. Then in-situ sequencing on dissected brain regions is used to detect barcodes in individual neurons, which represent the retrograde transportation downstream sources as well as the projection targets injected with detected barcodes. FIG. 13B shows demonstration of AAVretro racRNA barcode system in mapping projection targets of individual neurons in multiple brain regions. Nine kinds of barcoded racRNA were individually packaged into AAVretro and respectively injected into nine brain regions, including nucleus accumbens (NAc), basolateral amygdala (BLA), contralateral prefrontal cortex (cPFC), paraventricular nucleus of the thalamus (PVT), medial prefrontal cortex (mPFC), mediodorsal thalamus (MD), ventral tegmental area (VTA), Hypothalamus (Hypo) and dorsal periaqueductal gray (dPAG). The connection of neurons in these nine regions can be decoded by detecting barcodes, which are orthogonal to the locally injected barcode, in individual neurons. FIG. 13C shows example images showing the expression of AAVretro in the injection site (left) and retrogradely labeled upstream region (right). Dots in the images are expressed barcodes detected by in-situ sequencing.

FIG. 14 provides a schematic diagram providing a map of a racRNA-MS2-FingR-PSD95 (postsynapse) plasmid.

FIG. 15 provides a schematic diagram providing a map of a racRNA-PP7-VAMP2A plasmid.

FIG. 16 provides a schematic diagram providing a map of a racRNA-BC1 plasmid.

FIG. 17 provides a schematic diagram providing a map of a racRNA-hCTE-PP7 plasmid.

FIG. 18 provides a schematic diagram providing a map of a racRNA-30A-exporter-mCherry plasmid.

FIG. 19 provides a schematic diagram providing a map of a pcDNA-Myr-λN-Flag-4BoxB plasmid.

FIG. 20 provides a schematic diagram providing a map of a pcDNA-Pal-λN-Flag-4BoxB plasmid.

FIG. 21 provides a schematic diagram providing a map of a pcDNA-Flag-λN-Far-4BoxB plasmid.

FIG. 22 provides a schematic diagram providing a map of a pcDNA-Flag-MS2cp-Far-4MS2 plasmid.

FIG. 23 provides a schematic diagram providing a map of a pcDNA-Flag-PP7cp-Far-4PP7 plasmid.

FIG. 24 provides a schematic diagram providing a map of a pAAV-hSyn-Flag-λN-Far plasmid.

FIG. 25 provides a schematic diagram providing a map of a pAAV-hSyn-Flag-MS2cp-Far plasmid.

FIG. 26 provides a schematic diagram providing a map of a pAAV-hSyn-Flag-PP7cp-Far plasmid.

FIG. 27 provides a schematic diagram providing a map of a pAAV-U6-racRNA-BoxB-hSyn-Flag-λN-Far plasmid.

FIG. 28 provides a schematic diagram providing a map of a pAAV-U6-racRNA-MS2-hSyn-Flag-MS2cp-Far plasmid.

FIG. 29 provides a schematic diagram providing a map of a pAAV-U6-racRNA-PP7-hSyn-Flag-PP7cp-Far plasmid.

FIG. 30 provides a schematic diagram providing a map of a pAAV-U6-linear-PP7-hSyn-Flag-PP7cp-Far plasmid.

FIG. 31 provides a schematic diagram providing a map of a pAAV-U6-racRNA-PP7-hCTE-hSyn-Flag-PP7cp-Far plasmid.

FIG. 32 provides a schematic diagram providing a map of a pAAV-U6-racRNA-PP7-hSyn-V5-PP7cp-M9-NES plasmid.

FIG. 33 provides a schematic diagram providing a map of a pAAV-U6-racRNA-PP7-hSyn-V5-RtcB-3XNLS-T2A-Flag-PP7cp-Far plasmid.

FIG. 34 provides a schematic diagram providing a map of a pAAV-U6-racRNA-PP7-hSyn-V5-DDX39A-T2A-Flag-PP7cp-Far plasmid.

FIG. 35 provides a schematic diagram providing a map of a pAAV-U6-racBC1-hSyn-mCherry plasmid.

FIG. 36 provides a schematic diagram providing a map of a pAAV-U6-racBC200-hSyn-mCherry plasmid.

FIG. 37 provides a schematic diagram providing a map of a pAAV-U6-racRNA-PP7-hSyn-V5-PP7cp-M9-NES-Flag-PP7cp-Far plasmid.

FIG. 38 provides a schematic diagram providing a map of a pAAV-U6-racRNA-PP7-hCTE-hSyn-V5-PP7cp-M9-NES-Flag-PP7cp-Far plasmid.

FIG. 39 provides a schematic diagram providing a map of a pAAV-U6-racRNA-PP7-CAG-Flag-PP7cp-Far plasmid.

FIG. 40 provides a schematic diagram providing a map of a pAAV-U6-racRNA-PP7-CAG-V5-PP7cp-M9-NES-Flag-PP7cp-Far plasmid.

FIG. 41 provides a schematic diagram providing a map of a pAAV-U6-racRNA-PP7-hCTE-CAG-V5-PP7cp-M9-NES-Flag-PP7cp-Far plasmid.

FIG. 42 provides a schematic diagram providing a map of a pAAV-U6-racRNA-PP7-30A-hSyn-V5-PP7cp-M9-NES-Flag-PP7cp-Far plasmid.

FIG. 43 provides a schematic diagram providing a map of a pAAV-U6-racRNA-PP7-30A-hSyn-V5-PP7cp-M9-NES-mCherry-PP7cp-Far plasmid.

FIG. 44 provides a schematic diagram providing a map of a pAAV-U6-racRNA-PP7-30A-TRE-V5-PP7cp-M9-NES-mCherry-PP7cp-Far plasmid.

FIG. 45 provides a schematic diagram providing a map of a plasmid encoding a GB-M9 synaptic targeting construct corresponding to FIG. 9A.

FIG. 46 provides a schematic diagram providing a map of a plasmid encoding a GC-M9 synaptic targeting construct corresponding to FIG. 9A.

FIG. 47 provides a schematic diagram providing a map of a plasmid encoding a GD synaptic targeting construct corresponding to FIG. 9B.

FIG. 48 provides a schematic diagram providing a map of a plasmid encoding a GE1-M9 synaptic targeting construct corresponding to FIG. 9B.

FIG. 49 provides a schematic diagram providing a map of a plasmid encoding a GF1-M9 synaptic targeting construct corresponding to FIG. 9C.

FIG. 50 provides a schematic diagram providing a map of a plasmid encoding a GK synaptic targeting construct corresponding to FIG. 9D.

FIGS. 51A-51F provide images, a Uniform Manifold Approximation and Projection, cell type maps, and schematic diagrams showing a spatial chart of molecular cell types across the adult mouse central nervous system (CNS) at subcellular resolution. FIG. 51A provides a schematic diagram showing an overview of the study. After systemic administration of barcoded AAVs, mouse brain tissue slices were collected (top). STARmap PLUS (Wang, X. et al. Science 361, eaat 5691 (2018); Zeng, H. et al. Nat. Neurosci. (2023) doi:10.1038/s41593-022-01251-x) was performed to detect single RNA molecules from a targeted list of 1,022 endogenous genes and the trans-expressed AAV barcodes. The RNA spot matrix was converted to a cell-by-gene expression matrix via ClusterMap (He, Y. et al. Nat. Commun. 12, 5909 (2021)) (middle). By integrating with existing mouse brain single-cell RNA-seq data, a CNS spatial atlas was generated with cell cluster nomenclatures jointly defined by molecular cell types and molecular tissue regions, and imputed single-cell transcriptome-wide expression profiles (bottom). R.O., retro-orbital injection. FIG. 51B provides a Uniform Manifold Approximation and Projection (UMAP) of 1.09 million cells colored by subclusters. The surrounding diagrams show 230 subclusters from 26 main clusters. Top right, UMAP colored by slice directions; bottom right, UMAP colored by slice identity as in FIG. 51C. FIG. 51C provides molecular cell type maps of the 20 mouse CNS slices colored by subclusters. Each dot represents one cell. FIG. 51D provides a zoom-in view of tissue slice 12 in FIG. 51C. Each dot represents a DNA amplicon generated from an RNA molecule, color-coded by its cell-type identity. Brain regions abbreviations are based on the Allen Mouse Brain Reference Atlas. FIG. 51E provides a zoom-in view of the habenula region in FIG. 51D with cell boundaries outlined (left) and a mesh graph of physically neighboring cells connected via edges (middle), and symbols for cell types with >2 counts (right). Abbreviations: PEP, peptidergic neurons; CHO, cholinergic neurons; SER, serotonergic neurons; DOP, dopaminergic neurons; HA, histaminergic neurons; also see FIG. 51B. FIG. 51F provides a representative fluorescent image of the highlighted square region in FIG. 51E from the first SEDAL seq cycle. Each dot represents an amplicon.

FIGS. 52A-52D provide schematic diagrams and maps showing molecular tissue regions across the adult mouse CNS. FIG. 52A provides a schematic diagram showing a workflow of clustering molecular tissue regions by single-cell resolved spatial niche gene expression. A spatial niche gene expression vector of each cell was formed by concatenating its single-cell gene expression vector and those of the k nearest neighbors (kNNs) in physical space. The vectors of all cells were stacked into a spatial niche gene expression matrix and Leiden-clustered into molecular tissue regions. FIG. 52B provides an Allen Mouse Brain Common Coordinate Framework (CCFv3, 10 μm resolution) registration to facilitate molecular tissue region annotation. FIGS. 52C and 52D provide molecular tissue region maps registered into the visualizations in 3D (16 coronal and 3 sagittal slices combined, FIG. 52C) and 2D (individual slices, FIG. 52D). Representative registrations were shown to compare corresponding molecular tissue regions with anatomical tissue regions (anatomical outlines on top of molecular cell type maps) on the same slice (FIG. 52D, right). Each dot represents a cell. Anatomical region definitions were labeled in italics in blue. Tissue region abbreviations are based on the Allen Mouse Brain Reference Atlas (Dong, H. A Digital Color Brain Atlas of the C57BL 6JMale Mouse. (John Wiley and Sons, 2008); Allen Reference Atlas—Mouse Brain [brain atlas]. Available from atlas.brain-map.org).

FIGS. 53A and 53B provide schematic diagrams and a heatmap showing joint nomenclature of cell clusters through the combination of molecular cell types and molecular tissue regions. FIG. 53A provides schematics illustrating the workflow that combines molecular cell types and molecular tissue regions to jointly define cell type nomenclatures. FIG. 53B provides a heatmap showing the distribution of molecular cell types across molecular tissue regions. The cell-type percentage composition is calculated for each molecular tissue region. Then for each cell type, the z-scores of its percentages across regions are plotted. Subtypes of the same main cell type are grouped together. Molecular cell type abbreviations: HABCHO, habenular cholinergic neurons; HABGLU, habenular excitatory neurons; HBGLU, hindbrain excitatory neurons; HBINH, hindbrain inhibitory neurons; CBINH, cerebellar inhibitory neurons; CBGRC, cerebellar granule cells; CBPC: cerebellar Purkinje cells; also see FIG. 51B. In FIG. 53B, shown in each left panel is a top portion of a section of the heat map and shown in each right panel is the corresponding lower portion of the heat map.

FIGS. 54A-54D provide maps, plots, and schematic diagrams showing joint analysis and validation of molecular cell types in molecular tissue regions. FIGS. 54A and 54B provide from top-to-bottom: molecular tissue region maps, anatomical tissue maps registered to Allen CCFv3, marker cell type distribution maps, marker gene STARmap PLUS measurements, marker gene Allen Mouse Brain In Situ Hybridization (ISH) expression, and smFISH-HCR™ (single-molecule fluorescence in situ hybridization with hybridization chain reaction amplification) validation of molecular cortical superficial laminar structure (CTX_A_3-[L2/3]) within the anatomical cortical L2/3 (FIG. 54A) and anterior-posterior (from i to v) distribution of molecular retrosplenial (RSP) tissue regions (FIG. 54B). Cortical areas adjacent to RSP are labeled in the anatomical tissue maps. FIG. 54C provides plots showing Epha7 and Atp2b4 expression plotted in the UMAP of single-cell gene expression of dentate gyrus granule cells (DGGRC) (top) and that of spatial niche gene expression of molecular dentate gyrus (DG) regions (middle), and spatial niche gene expression UMAP colored by molecular cell types and molecular DG sublevel tissue regions (bottom). FIG. 54D provides a molecular tissue region map, molecular cell type map, and anatomical region map of DG granule cell layer (DGsg) (top) as well as STARmap PLUS measurements, Allen ISH expression (middle), and smFISH-HCR™ validation (bottom) of Epha7 and Atp2b4. smFISH-HCR™ images are representative of two (FIGS. 54A and 54D) or three experiments (FIG. 54B). Abbreviations: CTX, cerebral cortex; PL, prelimbic area; ACA, anterior cingulate area; MO, somatomotor areas; DGd-sg, dentate gyrus, granule cell layer, dorsal part; DGv-sg, dentate gyrus, granule cell layer, ventral part; SUB, subiculum; PRE, presubiculum; POST, postsubiculum. The ISH data were obtained from Allen Mouse Brain Atlas.

FIGS. 55A-55C provide schematic diagrams and maps showing transcriptome-scale adult mouse CNS spatial atlas by gene imputation. FIG. 55A provides schematics of the imputation workflow. Using the STARmap PLUS measurements and a scRNA-seq atlas as input, intermediate mappings were first performed by a leave-one-(gene)-out strategy. The resulting intermediate mappings were used to compute weights between STARmap PLUS identified cells and scRNA-seq cells for a final imputation to output 11,844-gene expression profiles in STARmap PLUS identified cells. FIG. 55B provides representative imputed spatial gene expression maps with corresponding STARmap PLUS and Allen Mouse Brain In Situ Hybridization (ISH) (Lein, E. S. et al. Nature 445, 168-176 (2007)) gene expression maps. Each dot represents a cell colored by the expression level of a gene. Scale bar, 0.5 mm. The sample slice number was labeled in gray. FIG. 55C provides maps showing examples of imputed spatial expression profile of selected genes outside the STARmap PLUS 1,022 gene list with the corresponding Allen ISH images. Scale bar, 1 mm. The ISH data were obtained from Allen Mouse Brain Atlas.

FIGS. 56A-56E provide schematic diagrams and images showing probe designs and raw fluorescent images of adult mouse CNS STARmap PLUS datasets. FIG. 56A provides a schematic diagram showing Mouse brain single-cell RNA-seq (scRNA-seq) sources for the STARmap PLUS 1,022 gene-list selection. FIG. 56B provides a schematic diagram showing SNAIL probes (primer and padlock probes) for 1,022 endogenous genes. The padlock probe contained a 5-nt gene-unique identifier, which was amplified during rolling-circle amplification and read out by six cycles of sequential SEDAL seq through adaptor sequence A. FIG. 56C, provides schematics showing the construct design and biogenesis of circular RNA barcodes. RtcB, RNA 2′,3′-cyclic phosphate and 5′-OH ligase. FIG. 56D provides a schematic diagram showing SNAIL probes for circular RNA barcodes. Each barcode was converted to a 1-nt identifier and read out by one additional cycle of SEDAL seq through adaptor sequence B. FIG. 56E provides Raw fluorescent images of SEDAL seq of brain slice 12. The left panels show the image stack maximum projection of SEDAL seq cycles 1 (top) and 7 (bottom), merged into an entire half slice. The top-right panels show zoom-in views of SEDAL seq cycles 1 to 7 and amplicons colored by gene identity from the square highlighted in the left panels. The bottom-right panels show the corresponding zoom-in views of the square highlighted in the top-right panels.

FIGS. 57A-57E provide schematic diagrams, dot plots, and bar graphs showing spatial cell typing workflow and data quality. FIG. 57A provides a schematic diagram showing data structure of the study and the workflow from raw images to a cell-by-gene matrix with cell spatial coordinates. Chs, channels. FIG. 57B provides bar graphs showing a summary of the number of tiles (i.e., imaging area), reads, and cells in each tissue sample slice. The number of cells is labeled on the figure. FIG. 57C, provides a schematic diagram showing a workflow of cell quality control, batch correction, and cell typing. Key parameters and thresholds were labeled. FIG. 57D provides dot plots of the top three marker genes for each main cluster. FIG. 57E provides dot plots showing main-cluster cell-type composition of each tissue sample slice as in absolute cell number (left) and cell fraction normalized within each tissue slice (right). M, medial; L, lateral; A, anterior; P, posterior. Data are provided in the accompanying Source Data file.

FIGS. 58A-58O provide images showing subclustering of main cell types. FIGS. 58A-58O show subcluster spatial maps on representative sample slices for astrocytes (FIG. 58A), oligodendrocytes and oligodendrocyte precursor cells (FIG. 58B), microglia (FIG. 58C), ependymal cells, choroid plexus epithelial cells, and subcommissural organ hypendymal cells (FIG. 58D), olfactory inhibitory neurons (FIG. 58E), cerebellum neurons (FIG. 58F), telencephalon projecting inhibitory neurons (FIG. 58G), di- and mesencephalon excitatory neurons (FIG. 58H), glutamatergic neuroblasts (FIG. 58I), non-glutamatergic neuroblasts (FIG. 58J), di- and mesencephalon inhibitory neurons (FIG. 58K), cholinergic and monoaminergic neurons (FIG. 58L), peptidergic neurons (FIG. 58M), hindbrain/spinal cord neurons (FIG. 58N), and vascular cells (FIG. 58O).

FIGS. 59A-59G provide images, a mesh graph, and a heatmap showing subclustering of telencephalon projecting excitatory neurons and telencephalon inhibitory interneurons, and spatial maps of representative subcluster cell types. FIGS. 59A and 59B provide images showing subcluster spatial maps on representative sample slices for telencephalon projecting excitatory neurons (TEGLU, FIG. 59A) and telencephalon inhibitory interneurons (TEINH, FIG. 59B). FIGS. 59C-59E provide images showing Cell-type spatial maps, zoom-in spatial expression heatmap of cell-type marker genes measured by STARmap PLUS, and corresponding In Situ Hybridization (ISH) images of the marker genes from the Allen Mouse Brain ISH database, for subcluster cell types HA_1 (FIG. 59C), HBGLU_2 and HABGLU_1 (FIG. 59D), and EPEN_1 and EPEN_2 (FIG. 59E). Each dot represents a cell color-coded by its subcluster cell-type symbol. Scale bars, 250 m if not indicated. FIG. 59F provides a mesh graph of cells shown on the STARmap PLUS molecular cell type map. Each cell is represented by a spot in the color of its corresponding main cell type. Physically neighboring cells are connected via edges. Zoom-in views of the top, middle, and bottom squares in the middle are shown on the right. FIG. 59G provides a heatmap showing first-tier cell-cell adjacency quantified by the normalized number of edges between individual pairs of main cell types (left). For each main cell type, the proportion of edges formed with cells of the same main type over the total number of edges with adjacent cells is shown in the bar plot (right). HA, histaminergic neurons; HBGLU, hindbrain excitatory neurons; HABGLU, habenular excitatory neurons; EPEN, ependymal cells; AC, astrocytes; MGL, microglia; DGGRC, dentate gyrus granule cells; DEGLU, diencephalon excitatory neurons.

FIGS. 60A-60E provide spatial plots and heatmaps showing brain anatomy registration (Allen CCFv3) and marker genes of molecular tissue regions. FIGS. 60A and 60B provide spatial plots of 20 sample slices colored by CCF anatomical labels according to the Allen Institute 3D Mouse Brain Atlas (Wang, Q. et al. Cell 181, 936-953.e20 (2020)) (FIG. 60A) and top-level molecularly defined tissue regions (FIG. 60B). Each dot represents a cell. FIG. 60C provides a heatmap showing the correspondence between main anatomical regions and top-level molecularly defined tissue regions. FIGS. 60D and 60E show marker gene heatmaps for top-level molecular tissue regions (top ten markers per region, ranked by z-scores of mean expression across regions, FIG. 60D) and sublevel molecular tissue regions (top three markers per region, ranked by z-scores of mean expression across regions, FIG. 60E). Tissue region abbreviations: OB, olfactory bulb; CTX, cerebral cortex; CBX, cerebellar cortex; CNU, cerebral Nuclei; TH, thalamus; HY, hypothalamus; MB_P_MY, midbrain, pons, and medulla; FT, fiber tracts; VS, ventricular systems; H, habenula; MYdp, medulla, dorsoposterior part; HPFmo, non-pyramidal area of hippocampal formation; MNG, meninges; ENTm, entorhinal area, medial part; HIP, Hippocampal region; DG, dentate gyrus; STR, striatum; CTXpl, cortical plate; CTXsp, cortical subplate; LSX, lateral septal complex; PAL, pallidum; HB, hindbrain; CBN, cerebellar nuclei. Data are provided in the accompanying Source Data file.

FIGS. 61A-61D provide heatmaps, spatial maps, and images showing molecular diversity within the cerebral cortex and the cerebellar cortex granular layer. FIG. 61A provides a spatial expression heatmap of representative marker genes for molecular cerebral cortical regions. FIG. 61B show molecular tissue regions, molecular cell types, and anatomical definition maps at the cerebellar cortex granule layer (top), spatial maps of molecular cerebellar cortex granule layer colored by the value of the first eigenvector of the diffusion map (DC1) (bottom left), and DC embeddings of spatial niche gene expression colored by molecular tissue region identities (bottom middle) or molecular cell type identities (bottom right). FIG. 61C provides images showing STARmap PLUS, Allen ISH (Lein, E. S. et al. Nature 445, 168-176 (2007)), and smFISH-HCR™ measurements of Adcy1 and Nrep that were enriched in the dorsal and ventral parts of the cerebellar cortex granular layer (CBX_1-[CBXd_gr] vs. CBX_3-[CBXv_gr]), respectively. FIG. 61D provides images showing a comparison of the molecular and anatomical tissue layer composition in various cortical regions covering the anterior-posterior, lateral-medial, and dorsal-ventral axes. Anatomical maps were shown as the registered tissue slices in CCFv3. Anatomical tissue region abbreviations: MO, somatomotor areas; MOs, secondary motor area; ACA, anterior cingulate area; PL, prelimbic area; AId, agranular insular area, dorsal part; AIp, agranular insular area, posterior part; ORB, orbital area; ILA, infralimbic area; RSP, retrosplenial area; RSPv, RSP ventral part; RSPagl, RSP lateral agranular part; RSPd, RSP dorsal part; SSp, primary somatosensory area; SSs, supplemental somatosensory area; VISC, visceral area; GU, gustatory areas; PIR, piriform area; VISp, primary visual area; VISl, lateral visual area; VISli, laterointermediate area; AUDp, primary auditory area; TEa, temporal association areas; ECT, ectorhinal area; ENT, entorhinal area; ENTI, ENT lateral part; PRE, presubiculum; POST, postsubiculum; IV-V, Culmen lobules IV-V; FL, flocculus.

FIGS. 62A-62C provide heatmaps showing cross-reference correspondence of STARmap PLUS main and subcluster cell types. Cell-type correspondence to cell types was annotated in single-cell RNA-seq datasets of adult mouse brain subregions including datasets on isocortex and hippocampus from the Allen Institute (FIG. 62A), ventral striatum (nucleus accumbens, FIG. 62B), and cerebellum (FIG. 62C). Cell type abbreviations: IT, intratelencephalic; PT, pyramidal tract; NP, near projecting. Data are provided in the accompanying Source Data file.

FIGS. 63A-63K provide heatmaps, plots, and images showing joint analysis and validation of molecular cell clusters in molecular tissue regions. FIG. 63A provides a heatmap showing the distribution of telencephalon inhibitory interneuron (TEINH) cell types across molecular telencephalon (TE) tissue regions. FIG. 63B provides a heatmap showing correspondence of interneuron subtypes within the molecular striatal tissue regions to intemeuron (IN) cell types annotated in the single-cell RNA-seq dataset of adult mouse ventral striatum (nucleus accumbens). FIGS. 63C-63E provide cell type maps overlaid on molecular tissue regions, spatial expression heatmaps of cell-type marker genes measured by STARmap PLUS, corresponding ISH images of the marker genes from the Allen Mouse Brain ISH database (Lein, E. S. et al. Nature 445, 168-176 (2007)), and independent smFISH-HCR™ validation of the distribution of the positive cells for TEINH_25 in the striatum (FIG. 63C) TEINH_10 and TEINH_22 in the olfactory bulb glomerular layer (OBopl, FIG. 63D), and TEINH_11 in cerebral cortical layer 2/3 (FIG. 63E). smFISH-HCR™ images are representative of two experiments (FIGS. 63C-63E). The ISH data were obtained from Allen Mouse Brain Atlas. FIG. 63F, UMAP embedding of OPC and OLG (left) and DC embedding (Haghverdi, L., et al. Bioinformatics 31, 2989-2998 (2015)) colored by molecular cell types (middle) and DC1 value (right). FIGS. 63G and 63I, Spatial distribution of DC1 values of the OPC-OLG lineage and OPC-OLG molecular cell cluster identities in the cerebral cortical layers (FIG. 63G) and midbrain-pons dorsal-ventral axis (FIG. 63I). FIG. 63H, DC1 values of the OPC-OLG lineage across the molecular cortical layers. Data shown as mean±s.t.d. FIG. 63J provides scatterplots showing DC embedding colored by marker gene expression levels indicating oligodendrocyte differentiation and maturation states. Only OPC and OLG cells are plotted (FIGS. 63G, 63I, and 63J). FIG. 63K provides a STARmap PLUS expression heatmap of Cxcl14, Rxfp1, and Neurod6 in representative coronal slices along the anterior-posterior axis.

FIGS. 64A-64E provide images and plots showing imputation parameter optimization and performance evaluation. FIG. 64A provides cumulative curves of the imputation performance scores across STARmap PLUS gene panels in the immediate mapping using different numbers of single-cell RNA-seq atlas cell nearest neighbors. The upper-left inset shows a zoom-in view of the rectangular region highlighted in the bottom right. Performance scores were calculated as the Pearson's correlation coefficient (PCC, across cells) between its imputed values and measured STARmap PLUS expression level. FIG. 64B provides scatter plots of spatial expression heterogeneity (Moran's I of the gene's spatial expression map) versus gene expression level in the STARmap PLUS datasets (left), and single-cell expression heterogeneity (Moran's I of scRNA-seq UMAP colored by the gene's expression) versus gene expression level in the scRNA-seq atlas (Zeisel, A. et al. Cell 174, 999-1014.e22 (2018)) (right). Each dot represents a gene and is colored by the gene's imputation performance score. n=1,016 genes. FIG. 64C provides images showing more examples of the comparison of imputed spatial gene expression with measured expression from STARmap PLUS and Allen Mouse Brain ISH database (Yao, Z. et al. Cell 184, 3222-3241.e26 (2021)). Each dot represents a cell colored by the expression level of a specified gene. Scale bar, 0.5 mm. The sample slice numbers were labeled in gray. FIGS. 64D-64E provide imputed spatial gene expression heatmaps of putative marker genes of the ventral part (FIG. 64D) and the dorsal part (FIG. 64E) of the medial habenula and the paired ISH images from the Allen Mouse Brain ISH database (Lein, E. S. et al. Nature 445, 168-176 (2007)).

FIGS. 65A-65F provide schematic diagrams, heatmaps, images, and boxplots showing AAV barcode quantification across molecular tissue regions and molecular cell types and validation. FIG. 65A provides schematics of AAV-PHP.eB tropism characterization strategy across the adult mouse CNS. vg, viral genome. FIG. 65B provides spatial heatmaps showing circular RNA expression on coronal slices. Each dot represents a cell color-coded by its AAV barcode expression level. FIGS. 65C and 65E provide boxplots of circular RNA expression level across molecular tissue regions (FIG. 65C) and main molecular cell types (FIG. 65E). Boxplot elements: the vertical line, median; the box, first to third quartiles; whiskers, 2.5-97.5%. Numbers in parentheses, number of cells in the group. Abbreviations for tissue region and cell type are the same as in the main figures. FIG. 65D presents schematics and images showing smFISH-HCR™ validation of AAV-PHP.eB tissue region tropisms. Images are representative of two experiments. The brain pictures were obtained from Allen Mouse Brain Atlas. FIG. 65F provides a heatmap showing a comparison of transduction rate observed in AAV-PHP.eB tropism profiling in the mouse isocortex via single-cell RNA-sequencing (Brown, D. et al. Front. Immunol. 12, 730825 (2021)) and the AAV RNA barcode expression in paired regions in the STARmap PLUS dataset. Anatomical tissue region abbreviations: STR, striatum; VL, lateral ventricle; LSX, lateral septal complex; CP, caudoputamen; ACB, nucleus accumbens; AI, agranular insular area; PAG, periaqueductal gray; PRN, pontine reticular nucleus; VIS, visual areas; PRE, presubiculum; ENT, entorhinal area; AQ, cerebral aqueduct; DR, dorsal nucleus raphe; SC, superior colliculus.

FIGS. 66A-66D provide a schematic diagram and plots showing STARmap PLUS sample collection and quality controls of cell clusters. FIG. 66A provides schematics of brain tissue collection in STARmap PLUS. The brain was quickly removed from the sacrificed animal and flash-frozen by liquid nitrogen to minimize disturbing tissue and RNA quality. FIG. 66B provides a scatter plot of the number of genes per cell versus the number of reads per cell in subclusters. n=230. FIGS. 66C and 66D provide scatter plots of the subcluster size (FIG. 66C, n=230) or subcluster population percentage in the main cluster (FIG. 66D, n=218, NA subclusters not included) versus the number of reads per cell (left) or the number of genes per cell (right). Each dot represents a cell subcluster; the median value of the cluster was plotted (FIGS. 66B-66D). Spearman's r and P values (two-tailed) were calculated with GraphPad Prism Version 9.3.1 (FIGS. 66B-66D).

FIGS. 67A-67N provide constellation plots and dot plots showing subclustering of main cell types. Uniform Manifold Approximation and Projection (UMAP) maps (left) and marker gene dot plots (right) of main clusters colored by cell subcluster identities, for astrocytes (AC, FIG. 67A), oligodendrocytes (OLG, FIG. 67B), microglia (MGL, FIG. 67C), ependymal cells (EPEN, FIG. 67D), olfactory inhibitory neurons (OBINH, FIG. 67E), cerebellum neurons (CB, FIG. 67F), telencephalon projecting inhibitory neurons (MSN, FIG. 67G), di- and mesencephalon excitatory neurons (FIG. 67H), cholinergic and monoaminergic neurons (FIG. 67I), peptidergic neurons (PEP or INH, FIG. 67J), di- and mesencephalon inhibitory neurons/hindbrain neurons/spinal neurons/unannotated (FIG. 67K), glutamatergic neuroblasts (FIG. 67L), and non-glutamatergic neuroblasts (FIG. 67M). FIG. 67N provides a marker gene dot plot for unannotated (NA) clusters. Dot sizes, the fraction of cells in the group; color bars, mean expression level in the group. Cell types and genes mentioned in the main text are bolded.

FIGS. 68A and 68B provide UMAP and constellation plots showing subclustering of telencephalon neurons and spatial maps of representative subcluster cell types. FIGS. 68A and 68B provide overlapped UMAP and constellation plots of main clusters colored by cell subcluster identities (left) and marker gene dot plots (right), for telencephalon projecting excitatory neurons (TEGLU, FIG. 68A) and telencephalon inhibitory interneurons (TEINH, FIG. 68B).

FIGS. 69A-69D provide boxplots showing imputation performance and gene expression features. FIGS. 69A-69D provide boxplots of imputation performance scores of genes of various expression features. Genes were divided into multiple groups based on their expression level in STARmap PLUS (FIG. 69A), spatial expression heterogeneity (FIG. 69B), expression level in the scRNA-seq atlas (FIG. 69C), or single-cell expression heterogeneity in the scRNA-seq atlas (FIG. 69D). PCC, Pearson's correlation coefficient between a gene's imputed values and measured STARmap PLUS expression level across cells. P values were calculated with two-sided Mann-Whitney-Wilcoxon tests. **P<0.01, ***P<0.001, ****P<0.0001. Numbers in parentheses, number of genes.

### DETAILED DESCRIPTION

The disclosure features, among other things, compositions, systems, and methods for preparation and use of efficient RNA nuclear export of ribozyme-assisted circular RNA molecules (racRNAs). In embodiments, the methods involve characterizing a cell or tissue.

The aspects and embodiments of the disclosure are based, at least in part, upon the discovery detailed in the Examples provided herein of methods for enabling efficient export of ribozyme-assisted circular RNA molecules (racRNAs) from the cell nucleus. In embodiments, the methods of the disclosure harness endogenous RNA nuclear export pathways to export RNA from the nucleus and/or involve binding of the racRNAs to RNA-binding polypeptides to localize the racRNAs to defined subcellular compartments. The methods, systems, and compositions provide herein allow for efficient export from the nucleus of racRNAs that function in the cytoplasm.

The aspects and embodiments of the disclosure are also based, at least in part, upon the development of an in situ sequencing method using STARmap PLUS (Wang, X. et al. Science 361, eaat 5691 (2018); Zeng, H. et al. Nat. Neurosci. (2023) doi:10.1038/s41593-022-01251-x), to profile 1,022 genes in 3D at a voxel size of 194×194×345 nm³, mapping 1.09 million high-quality cells across the adult mouse brain and spinal cord. Spatially charting molecular cell types at single-cell resolution across the three-dimensional (3D) volume is critical for illustrating the molecular basis of brain anatomy and functions. Single-cell RNA sequencing has profiled molecular cell types in the mouse brain, but cannot capture their spatial organization. Computational pipelines were developed to segment, cluster, and annotate 230 molecular cell types by single-cell gene expression and 106 molecular tissue regions by spatial niche gene expression. Joint analysis of molecular cell types and molecular tissue regions enabled a systematic molecular spatial cell type nomenclature and identified tissue architectures undefined in established brain anatomy. To create a transcriptome-wide spatial atlas, STARmap PLUS measurements were integrated with a published scRNA-seq atlas, imputing single-cell expression profiles of 11,844 genes. Finally, viral tropisms were delineated for a brain-wide transgene delivery tool, AAV-PHP.eB (Chan, K. Y. et al. Nat. Neurosci. 20, 1172-1179 (2017); Goertsen, D. et al. Nat. Neurosci. 25, 106-115 (2022)). Together, this annotated dataset provides a comprehensive single-cell resource that integrates the molecular spatial atlas, brain anatomy, and genetic manipulation accessibility of the mammalian central nervous system (CNS).

### RNA Export

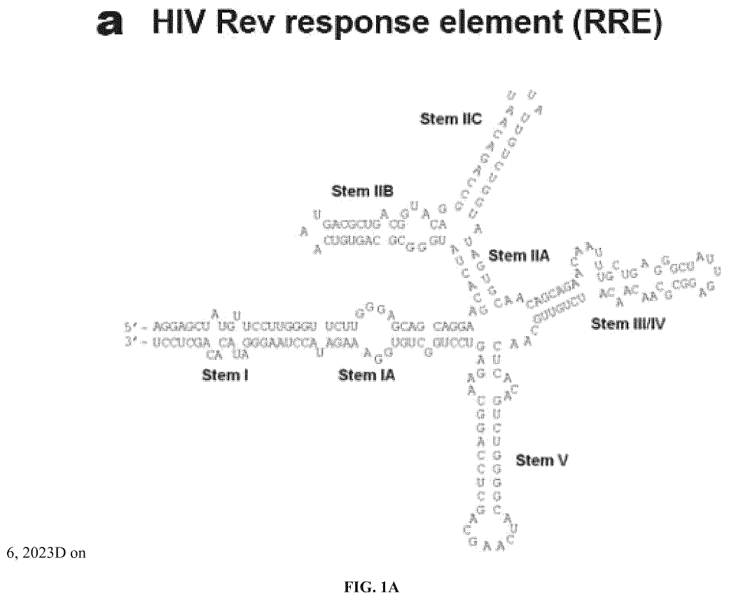

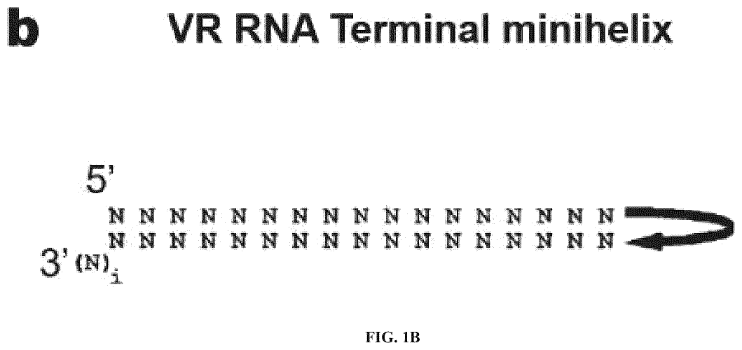

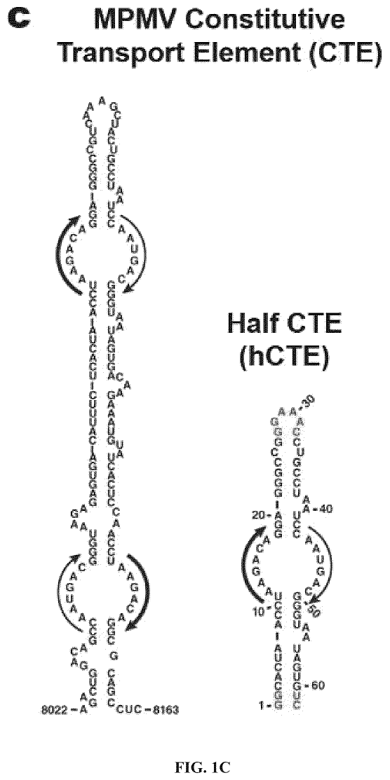

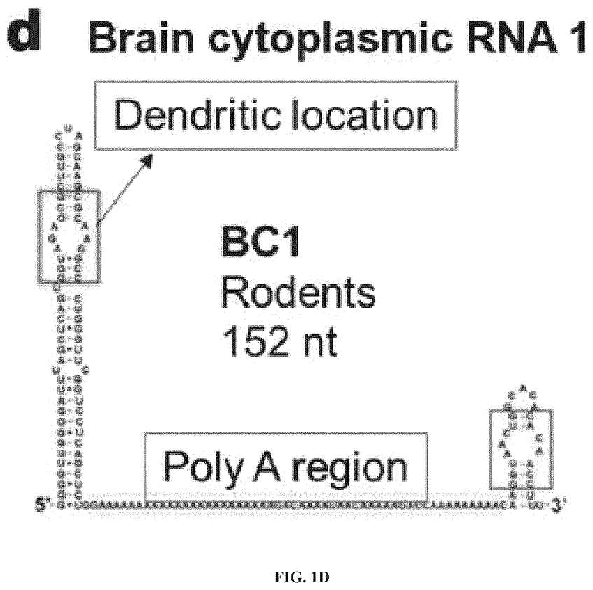

Studies of how viral RNA is exported from the nucleus to the cytoplasm has shed light on the mechanism of eukaryotic RNA export, which is regulated through the nuclear pore complex (Okamura M, et al. “RNA export through the NPC in eukaryotes,” Genes (Basel) 6:124-149. 2015). RNA motifs (e.g., RNA hairpins) recognized by host cell nuclear export machinery have been identified in viral genomes. For example, while the mRNA export pathway rejects most un-spliced RNAs, intron-containing HIV RNA with the Rev response element (RRE) (FIG. 1A) is exported when the HIV protein Rev adapts it to the host export receptor CRM1. Also, short RNA elements enable the export of adenovirus VA1 RNA (Terminal minihelix) (FIG. 1B) and of Mason-Pfizer Monkey Virus transcripts (MPMV) (Constitutive Transport Element, CTE) (FIG. 1C) from the cell nucleus. Typically, non-coding RNAs are retained in the nuclei. Besides ribosomal RNAs and transfer RNAs, which are exported from the nucleus for protein synthesis, another RNA exported from the nucleus of a cell is the brain cytoplasmic RNA (BC1 in rodents and BC200 in primates), a neuron-specific non-coding RNA (ncRNA) (FIG. 1D).

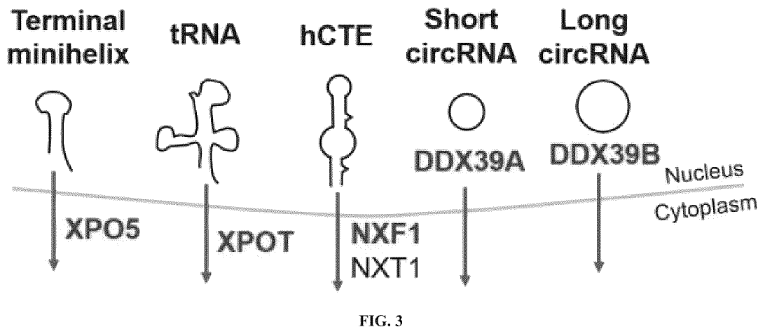

Important proteins in the nuclear export pathway of various RNAs are shown in FIG. 3. For example, the terminal minihelix is exported through the major export pathway of microRNAs, specifically the nuclear export receptor XPO5. Also, hCTE is recognized by the NXF1, one of the components of the mRNA export receptor heterodimer NXF1/NXT1. For circular RNAs (circRNAs), an RNAi screening study in fruit flies identified length-dependent export through different export adaptors: the export of short circRNA (<400 nt) depends on DDX39A while the longer ones (>1000 nt) depend on DDX39B. In various embodiments, the abundance of the export mediators can be enhanced if there is not sufficient endogenous expression in cell types of interest.

Besides interacting with RNA export adaptors and receptors for export, RNA can also be exported with protein partners in the form of RNA-protein complexes. Some of the RNA binding proteins (RBPs) shuttle between the nuclei and the cytoplasm, regulating the nuclear-cytoplasmic distribution of their RNA targets. Among those proteins, heterogeneous nuclear ribonucleoprotein A1 (hnRNP A1) is a well-studied shuttling RBP. An approximate 40 amino acid M9 sequence in the protein signals the shuttling by interacting with protein export and import receptors at the NPC.

### Ribozyme-Assisted Circular RNAs

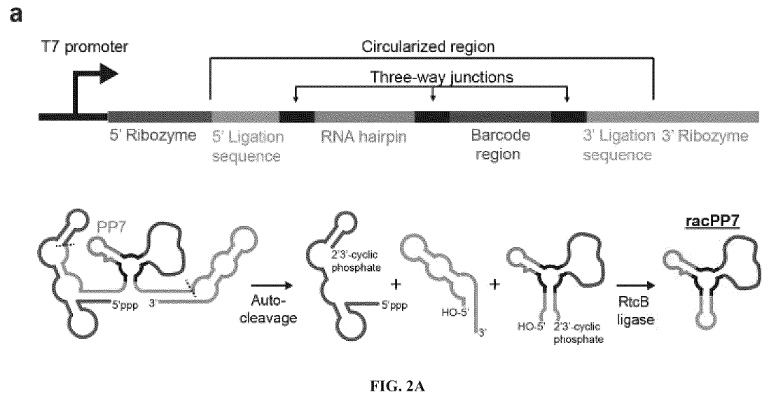

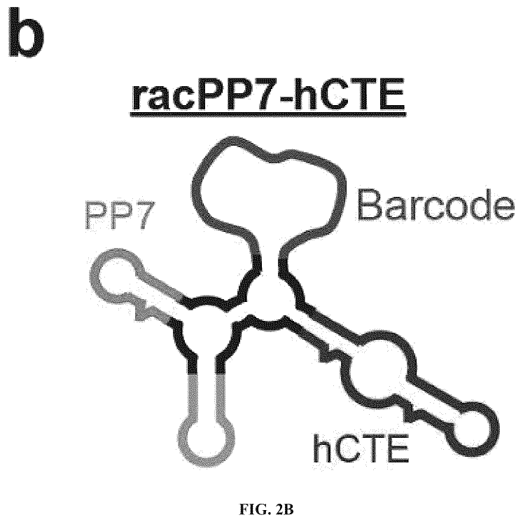

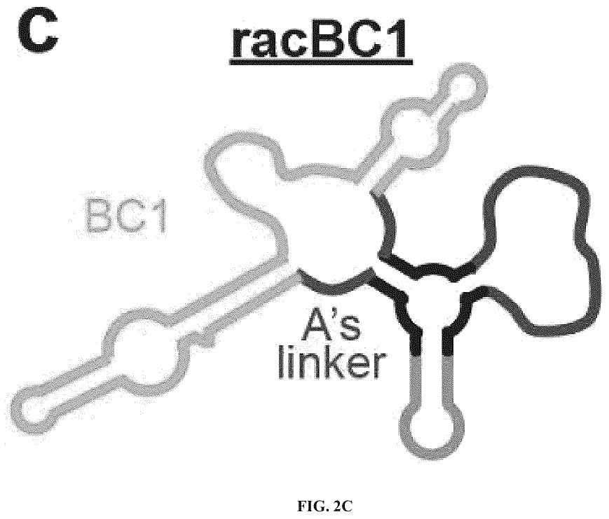

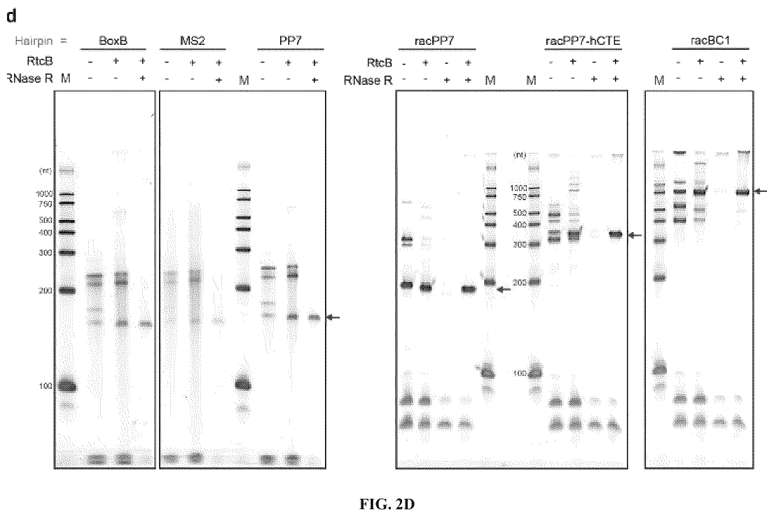

In various aspects, the present disclosure provides ribozyme-assisted circular RNAs (racRNAs) and vectors and/or polynucleotides encoding the same. A schematic overview of an exemplary embodiment of a polynucleotide encoding a racRNA is provided in FIG. 2A. A racRNA comprises two ribozymes (a 5′ ribozyme and a 3′ ribozyme) flanking a circularizing region (see, e.g., US Patent Application Publication No. 2021/034052, the disclosure of which is incorporated herein by reference in its entirety for all purposes). The circularizing region contains at the 5′ terminus thereof a 5′ ligation sequence and at the 3′ terminus thereof a 3′ ligation sequence. Upon self-ligation of the 5′ ribozyme and 3′ ribozyme in a cell, the 5′ ligation sequence and the 3′ ligation sequence together form a stem structure. Following self-ligation of the 5′ ribozyme and 3′ ribozymes in the cell, the 5′ ligation sequence is ligated to the 3′ ligation sequence by an RNA ligase (e.g., a tRNA processing ligase, or an ATP-dependent RNA ligase, such as RtcB). The circularizing region contains a payload region containing an RNA hairpin capable of binding an RNA binding polypeptide.

Non-limiting examples of self-cleaving ribozymes suitable for use in the racRNAs of the disclosure include any self-cleaving ribozyme known in the art, such as those provided herein and/or described in Tang and Breaker, “Structural diversity of self-cleaving ribozymes,” Proc Natl Acad Sci USA, 97:5784-5789 (2000); or in Weinberg, et al. “Novel ribozymes: discovery, catalytic mechanisms, and the quest to understand biological function,” Nucleic Acids Research, 47:9480-9494 (2019), the disclosures of which are incorporated herein by reference in its entirety for all purposes.

In one embodiment, each of the 5′ ribozyme and the 3′ ribozyme comprise a sequence that may be cleaved to produce a 5′-OH end and a 2′,3′-cyclic phosphate end. In accordance with this embodiment, each of the 5′ ribozyme and the 3′ ribozyme is a self-cleaving ribozyme. Self-cleaving ribozymes are characterized by distinct active site architectures and divergent, but similar, biochemical properties. The cleavage activities of self-cleaving ribozymes are highly dependent upon divalent cations, pH, and base-specific mutations, which can cause changes in the nucleotide arrangement and/or electrostatic potential around the cleavage site (see, e.g., Weinberg et al., “New Classes of Self-Cleaving Ribozymes Revealed by Comparative Genomics Analysis,” Nat. Chem. Biol. 11(8): 606-610 (2015) and Lee et al., “Structural and Biochemical Properties of Novel Self-Cleaving Ribozymes,” Molecules 22(4):E678 (2017), which are hereby incorporated by reference in their entirety for all purposes).

Suitable self-cleaving ribozymes include, but are not limited to, Hammerhead, Hairpin, Hepatitis Delta Virus (“HDV”), Neurospora Varkud Satellite (“VS”), Vg1, glucosamine-6-phosphate synthase (glmS), Twister, Twister Sister, Hatchet, Pistol, and engineered synthetic ribozymes, and derivatives thereof (see, e.g., Harris et al., “Biochemical Analysis of Pistol Self-Cleaving Ribozymes,” RNA 21(11):1852-8 (2015), which is hereby incorporated by reference in its entirety for all purposes).

Twister ribozymes comprise three essential stems (P1, P2, and P4), with up to three additional ones (P0, P3, and P5) of optional occurrence. Three different types of Twister ribozymes have been identified depending on whether the termini are located within stem P1 (type P1), stem P3 (type P3), or stem P5 (type P5) (see, e.g., Roth et al., “A Widespread Self-Cleaving Ribozyme Class is Revealed by Bioinformatics,” Nature Chem. Biol. 10(1):56-60 (2014), the disclosure of which is incorporated herein by reference in its entirety for all purposes). The fold of the Twister ribozyme is predicted to comprise two pseudoknots (T1 and T2, respectively), formed by two long-range tertiary interactions (see Gebetsberger et al., “Unwinding the Twister Ribozyme: from Structure to Mechanism,” WIREs RNA 8(3):e1402 (2017), the disclosure of which is hereby incorporated by reference in its entirety for all purposes).

Twister Sister ribozymes are similar in sequence and secondary structure to Twister ribozymes. In particular, some Twister RNAs have P1 through P5 stems in an arrangement similar to Twister Sister and similarities in the nucleotides in the P4 terminal loop exist. However, these two ribozyme classes cleave at different sites, Twister Sister ribozymes do not appear to form pseudoknots via Watson-Crick base pairing (which occurs in all known twister ribozymes), and there is poor correspondence among many of the most highly conserved nucleotides in each of these two motifs (see Weinberg et al., “New Classes of Self-Cleaving Ribozymes Revealed by Comparative Genomics Analysis,” Nat. Chem. Biol. 11(8):606-610 (2015), which is hereby incorporated by reference in its entirety).

Pistol ribozymes are characterized by three stems: P1, P2, and P3, as well as a hairpin and internal loops. A six-base-pair pseudoknot helix is formed by two complementary regions located on the P1 loop and the junction connecting P2 and P3; the pseudoknot duplex is spatially situated between stems P1 and P3 (Lee et al., “Structural and Biochemical Properties of Novel Self-Cleaving Ribozymes,” Molecules 22(4):E678 (2017), which is hereby incorporated by reference in its entirety for all purposes).

Hammerhead ribozymes are composed of structural elements including three helices, referred to as stem I, stem II, and stem III, and joined at a central core of 11-12 single strand nucleotides. Hammerhead ribozymes may also contain loop structures extending from some or all of the helices. These loops are numbered according to the stem from which they extend (e.g., loop I, loop II, and loop III).

In one embodiment, the 5′ ribozyme is a Twister ribozyme or a Twister Sister ribozyme. For example, the 5′ ribozyme may be a P3 Twister ribozyme.

In another embodiment, the 3′ ribozyme is a Twister, Twister Sister, or Pistol Ribozyme. For example, the 3′ ribozyme may be a P1 Twister ribozyme.

In one embodiment, the 5′ ribozyme is a P3 Twister ribozyme and the 3′ ribozyme is a P1 Twister ribozyme.

The ribozymes of the present invention include naturally-occurring (wildtype) ribozymes and modified ribozymes, e.g., ribozymes containing one or more modifications, which can be addition, deletion, substitution, and/or alteration of at least one (or more) nucleotide. Such modifications may result in the addition of structural elements (e.g., a loop or stem), lengthening or shortening of an existing stem or loop, changes in the composition or structure of a loop(s) or a stem(s), or any combination of these. As described herein, modification of the nucleotide sequence of naturally occurring self-cleaving ribozymes (e.g., a P3 Twister ribozyme) can increase or decrease the ability of a ribozyme to autocatalytically cleave its RNA. In one embodiment, each of the first and the second ribozyme is, independently, modified to comprise a non-natural or modified nucleotide. In some embodiments, each of the first and the second ribozyme is modified to comprise pseudouridine in place of uridine.

In another embodiment, each of the 5′ and the 3′ ribozyme is, independently, a split ribozyme or ligand-activated ribozyme derivative.

Methods of producing a ribozyme targeted to a target sequence are known in the art. Ribozymes may be designed as described in PCT Publication No. WO 93/23569 and PCT Publication No. WO 94/02595, each of which is hereby incorporated by reference in its entirety, and synthesized to be tested in vitro and in vivo, as described therein.

The racRNA may contain 1, 2, 3, 4, 5, or more RNA motifs (e.g., RNA hairpins) capable of binding an RNA binding polypeptide. In embodiments, the RNA motif forms an RNA hairpin. Non-limiting examples of RNA motifs suitable for use in the racRNAs include a BC1, a BC200, a BoxB, an hCTE, an MS2, a PP7, an HIV Rev response element, a VR RNA terminal minihelix, and an MPMV constitutive transport element (CTE). In some instances, the racRNA comprises a PP7 motif and an hCTE motif. In some instances, the RNA motif is an RNA motif bound by a viral capsid protein selected from one or more of MS2, PP7, Qβ, F2, GA, fr, JP501, M12, R17, BZ13, JP34, JP500, KU1, Mi 1, MX1, TW18, VK, SP, FI, ID2, NL95, TW19, AP205, φCb5, φCb8r, φCbl2r, φCb23r, 7s and PRR1.

The racRNA may contain one or more of an RNA sequence that binds a protein; an RNA sequence that is complementary to a microRNA or siRNA; an RNA sequence that has partial complementarity to a microRNA or siRNA or piRNA; an RNA sequence that hybridizes completely or partially to a cellularly expressed microRNA, siRNA, piRNA, mRNA, lncRNA, ncRNA, or other cellular RNA; a hairpin structure that is a substrate for DICER or endogenous nucleases; a sequence that binds to viral proteins; an antisense RNA, an antagomir, a microRNA, an siRNA, an anti-miRNA, a ribozyme, a decoy oligonucleotide, an RNA activator, an immunostimulatory oligonucleotide, an aptamer, an RNA device; and an RNA molecule encoding a peptide sequence.

The racRNA may contain an RNA aptamer that binds with high affinity and specificity to a target. RNA aptamers may be single-stranded, partially single-stranded, partially double-stranded, or double-stranded nucleotide sequences. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

The RNA aptamer may comprise a fluorogenic aptamer. Fluorogenic aptamers are well known in the art and include, without limitation, Spinach, Spinach 2, Broccoli, Red-Broccoli, Orange Broccoli, Corn, Mango, Malachite Green, cobalamine-binding aptamer, and derivatives thereof. See, e.g., Autour et al., “Fluorogenic RNA Mango Aptamers for Imaging Small Non-Coding RNAs in Mammalian Cells,” Nature Comm. 9: Article 656 (2018); Jaffrey, S., “RNA-Based Fluorescent Biosensors for Detecting Metabolites In Vitro and in Living Cells,” Adv Pharmacol. 82:187-203 (2018); and Litke et al., “Developing Fluorogenic Riboswitches for Imaging Metabolite Concentration Dynamics in Bacterial Cells,” Methods Enzymol. 572:315-33 (2016), each of which are hereby incorporated by reference in its entirety for all purposes). In accordance with this embodiment, the fluorogenic aptamer binds to a fluorophore whose fluorescence, absorbance, spectral properties, or quenching properties are increased, decreased, or altered by interaction with the fluorogenic aptamer. Any aptamer-dye complex, some of which are fluorogenic aptamers, may be used. In addition, some aptamers can bind quenchers and some do other things to change the photophysical properties of dyes.

In another embodiment, the aptamer binds a target molecule of interest. The target molecule of interest may be any biomaterial or small molecule including, without limitation, proteins, nucleic acids (RNA or DNA), lipids, oligosaccharides, carbohydrates, small molecules, hormones, cytokines, chemokines, cell signaling molecules, metabolites, organic molecules, and metal ions. The target molecule of interest may be one that is associated with a disease state or pathogen infection. As demonstrated in the accompanying Examples, circular aptamers directed against a target molecule of interest can be developed to inhibit a cellular signaling pathway, e.g., the NF-κB signaling.

In some embodiments, the racRNA contains a fluorogenic aptamer coupled to an aptamer that binds a target molecule of interest. In accordance with this embodiment, the racRNA molecule may be a sensor. In accordance with this embodiment of the invention, the fluorogenic aptamer is coupled to an aptamer that binds a target molecule using a transducer stem. Suitable target molecules of interest include, but are not limited to, ADP, adenosine, guanine, GTP, SAM, and streptavidin. As demonstrated in the accompanying Examples, circular aptamer “sensors” can be developed, e.g., against SAM.

In some instances, the payload region further comprises a barcode for uniquely identifying the racRNA. In various embodiments, the barcode comprises a nucleotide sequence that is about or at least about 1, 2, 3, 4, 5, 6, 7, 8, 9, 10, 11, 12, 13, 14, 15, 16, 17, 18, 19, or 20 nucleotides in length. In various embodiments, the barcode comprises a nucleotide sequence that is no more than about 3, 4, 5, 6, 7, 8, 9, 10, 11, 12, 13, 14, 15, 16, 17, 18, 19, or 20 nucleotides in length. In some cases, the barcode is 3′ of the RNA motif.

In some embodiments, the payload region comprises an RNA segment or polynucleotide of interest. In embodiments, the RNA segment or polynucleotide of interest is about or at least about 5, 10, 15, 20, 25, 30, 35, 40, 45, 50, 75, 100, 150, 200, 250, 300, 350, 400, 450, 500, 750, or 1000 nucleotides in length. In embodiments, the RNA segment or polynucleotide of interest is no more than about 5, 10, 15, 20, 25, 30, 35, 40, 45, 50, 75, 100, 150, 200, 250, 300, 350, 400, 450, 500, 750, or 1000 nucleotides in length. In embodiments, the RNA segment or polynucleotide of interest is complementary to a polynucleotide sequence present in the genome of a cell or to a polynucleotide present in a cell (e.g., in the nucleus or cytoplasm). In embodiments, the RNA segment or polynucleotide of interest is 3′ of the RNA motif.

In some cases, it is advantageous for the racRNA to contain a stretch of adenines (As). In embodiments, the stretch of As is about or at least abut 5, 10, 15, 20, 25, 30, 35, 40, 45, 50, 75, or 100 nucleotides in length. In embodiments, the stretch of As is no more than about 10, 15, 20, 25, 30, 35, 40, 45, 50, 75, or 100 nucleotides in length. The stretch of As can be located anywhere within the racRNA molecule. In some instances, the stretch of As is 3′ or 5′ of the RNA motif. In some cases, the stretch of As is 3′ of a barcode, RNA segment, or polynucleotide of interest. In some cases, the stretch of As is adjacent to the barcode, RNA segment, or polynucleotide of interest.

In some instances, the racRNA contains junctions separating different elements of the racRNA. In embodiments, each junction is independently about or at least about 5, 10, 15, 20, 25, 30, 35, 40, or 50 nucleotides in length. In embodiments, each junction is independently less than about 5, 10, 15, 20, 25, 30, 35, 40, or 50 nucleotides in length. In embodiments, a junction separates the 5′ ligation sequence from an RNA motif. In embodiments, a junction separates the RNA motif from an RNA segment, polynucleotide of interest, or barcode. In embodiments, a junction separates an RNA segment, polynucleotide of interest, or barcode from a 3′ ligation sequence. In embodiments, a junction separates the stretch of As from the 3′ ligation sequence.

In one embodiment, the first ligation sequence (e.g., a 5′ ligation sequence) and the second ligation sequence (e.g., a 3′ ligation sequence) are substrates for an RNA ligase. According to one embodiment, the RNA ligase is RtcB. RtcB is not present in all lower organisms, but molecules with similar activities are present. In other words, there are molecules that ligate ends similar to the ligation activity of RtcB. RtcB (or other functionally similar molecules) may be overexpressed to maximize circular RNA expression.

An advantage of the ligation sequence is to assist in circularization of the RNA molecule, to protect the RNA molecule from degradation and, therefore, ultimately enhance expression of the RNA molecule. While it is thought that the RNA molecule of the present invention could circularize without the ligation sequences, and such an invention is contemplated, the ligation sequences are also believed to cause the RNA ends to come together more efficiently for the RNA ligase (e.g., RtcB). In other words, the ligation sequences are believed to help draw proper 5′ and 3′ ends of the RNA molecule closer to each other to assist in the circularization of the RNA molecule.

In embodiments, the present disclosure provides polynucleotides encoding a racRNA. In embodiments, the racRNA is expressed under the control of a promoter. Promoters suitable for use in embodiments of the polynucleotides of the disclosure include any promoter described herein. In various instances, the promoter is a U6 promoter or a T7 promoter.

Non-limiting examples of embodiments of racRNAs include those described in FIGS. 2A, 2B, 2C, 5B-5G, 6B-6C, 7A-7C, and 8A-8G.

In an embodiment, the racRNA is synthesized (e.g., by chemical synthesis) or in vitro by transcribing the RNA, allowed to self-process via the ribozymes, and then incubated with purified RtcB. Circular RNA is then purified by standard methods. The purified circular RNA may then be administered to a person or cell, e.g., for treatment purposes.

According to another embodiment a racRNA molecule of the present disclosure is expressed from a genome or from a plasmid or a phage. In one embodiment, such RNA expression is accompanied by overexpression of RtcB (or another suitable RNA ligase). According to this embodiment, it would be possible to manufacture large quantities of circular RNA (e.g., in E. coli) for subsequent purification.

### RNA-Binding Polypeptides

In various aspects, the disclosure features vectors and polynucleotides encoding an RNA-binding polypeptide. In some aspects, the methods of the disclosure involve co-expressing one or more RNA-binding polypeptides and/or an RNA ligase, and an ribozyme-assisted circularized RNA (racRNA) in a cell.

In some cases, the RNA-binding polypeptide is an RNA transport protein. Non-limiting examples of RNA transport proteins include RNA export receptors, such as XPO5, XPOT, NXF1, NXT1, DDX39A, and DDX39B.

In some cases, the vectors and polynucleotides of the present disclosure further encode an RNA ligase (e.g., RtcB).

In some instances, the RNA-binding polypeptide comprises one or more of the following RNA binding domains a PP7cp, a tandem PP7 capsid protein domain (tdPP7cp), a tandem MS2 capsid protein domain (MS2cp), λN. In some cases, the RNA binding domain is fused to one or more nuclear export sequences (e.g., an M9 tag). In some instances, the RNA binding domain is fused to a polypeptide that localizes to a cellular compartment (e.g., a famesylation (Far) motif, VAMP2A, SYP1, homer1c, PSD95 FingR domain, GPHN FingR domain, ARC). In embodiments the polypeptide that localizes to a cellular compartment localizes to a pre-synapse compartment of a cell (e.g., VAMP2A or SYP1), to an excitatory post-synapse compartment of a cell (e.g., homer1c), to an inhibitory post-synapse compartment (e.g., FingR of GPHN), to dendritic spines, or pan-dendritic compartments (e.g., ARC). In embodiments, a racRNA comprising a BC1 motif is used to localize a barcode, polynucleotide of interest, or RNA segment contained within the racRNA to pan-dendritic compartments of a cell. In embodiments, the polypeptide that localizes to a cellular compartment is a human protein or a rat protein. In embodiments, the methods of the disclosure involve localizing a racRNA molecule to a cellular compartment of a neuron selected from the group consisting of nucleus, cytoplasm, soma, neurites, and/or dendrites, or combinations thereof. In some instances, the RNA-binding polypeptide contains a viral coat protein or a functional fragment thereof, wherein the viral coat protein is selected from one or more of Examples of such coat proteins include but are not limited to: MS2, PP7, Qβ, F2, GA, fr, JP501, M12, R17, BZ13, JP34, JP500, KU1, Mi 1, MX1, TW18, VK, SP, FI, ID2, NL95, TW19, AP205, φCb5, φCb8r, φCbl2r, φCb23r, 7s and PRR1.

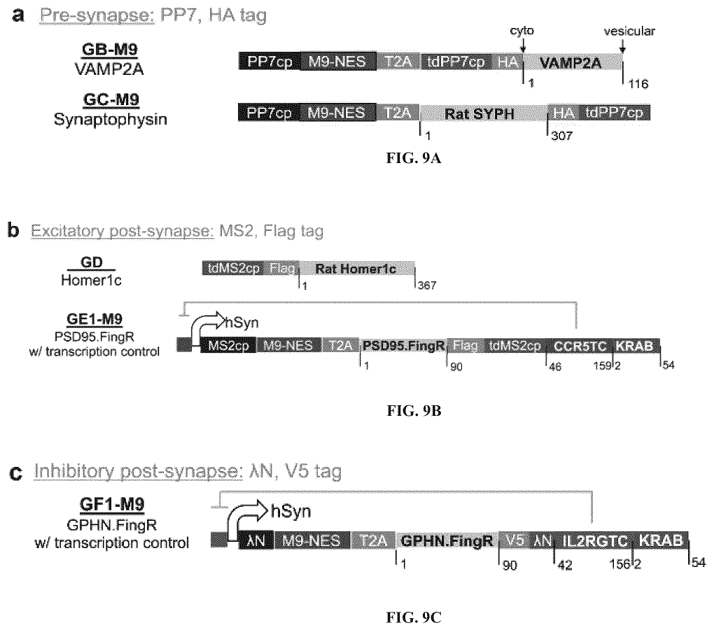

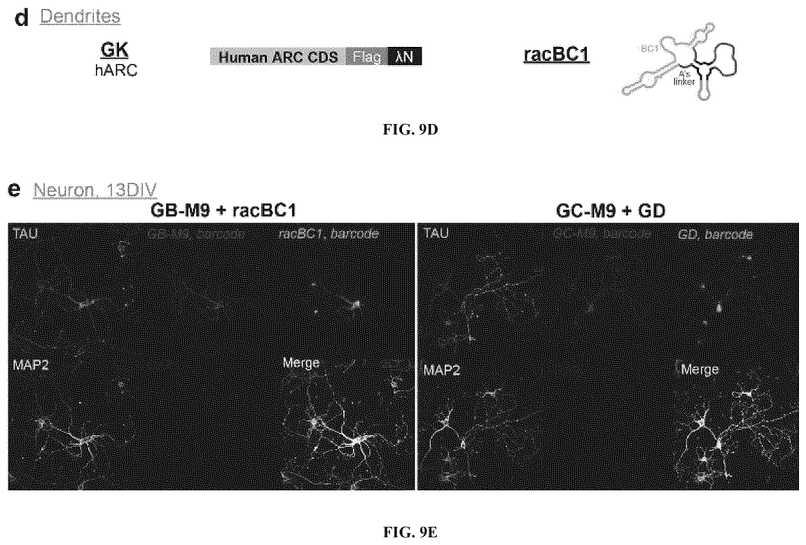

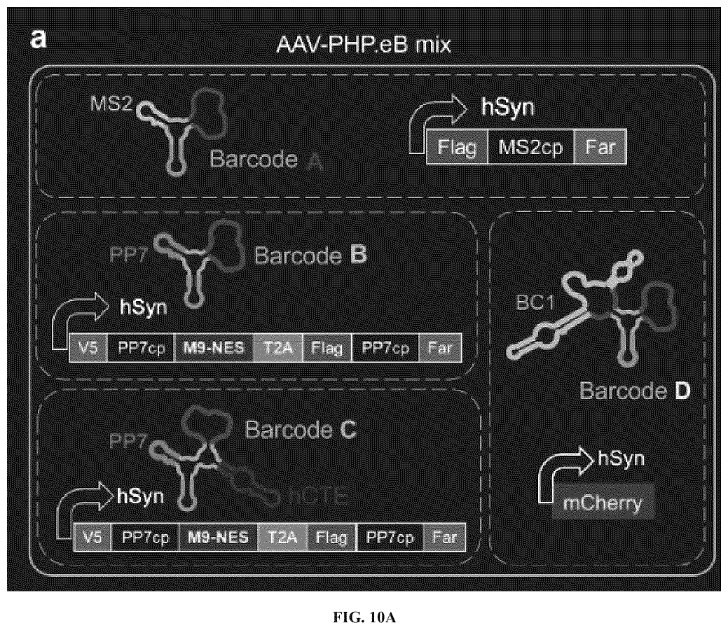

In various embodiments, it can be advantageous to place expression of an racRNA from a polynucleotide under the control of negative-feedback transcriptional control. For example, such control may be achieved using a construct as shown in FIG. 9B or 9C. In an embodiment, the negative-feedback transcriptional control involves placing expression of a repressor protein, a racRNA, and, optionally, one or more further polypeptides, under the control of a promoter downstream of a nucleotide sequence to which the repressor protein binds to effectively repress expression of the racRNA. In various embodiments, the repressor protein is IL2RGTC fused to KRAB or CCR5TC fused to KRAB. The CCR5TC domain contains a DNA sequence recognizing CCR5 zinc finger protein fused to a KRAB(A) transcriptional repressor domain. IL2GTC contains a DNA sequence recognizing CCR5 zinc finger protein. In embodiments, a method of the disclosure involves expressing an racRNA and FingR of GPHN or FingR of PSD95 using the negative-feedback transcriptional control. In embodiments, expression of the racRNA and the FingR of GPHN fused to an RNA binding polypeptide or the FingR of PSD95 fused to an RNA binding polypeptide under the control of the negative-feedback transcriptional control allows for specific localization of the racRNA to dendritic spines.

In embodiments, the polynucleotides of the disclosure further encode a fluorescent protein, such as GFP or mCherry. In embodiments, the polynucleotides of the disclosure encode a polypeptide fused to an epitope tag, such as a FLAG tag, a V5 tag, or an HA tag, suitable for visualization using various immunostaining techniques known in the art.

In various embodiments, a polypeptide of the disclosure is fused to a nuclear localization signal (NLS) and/or to a nuclear export signal (NES). In embodiments, the polypeptide is fused to 1, 2, 3, 4, or 5 nuclear localization and/or nuclear export signals (e.g., 3×NES). In various cases, the NLS or NES is located at a C-terminus of a polypeptide encoded by a polynucleotide of the disclosure and/or is just N-terminal of a self-cleaving peptide.

In some cases, a polynucleotide of the disclosure encodes one or more polypeptides translated as a single molecule that is then cleaved at self-cleaving polypeptides separating each of the polypeptides. Non-limiting examples of self-cleaving polypeptides include T2A, P2A, E2A, and F2A.

Characterization of Cells and/or Tissues

In embodiments, the methods of the invention involve determining the localization in a cell or tissue of one or more of the racRNA polynucleotides provided herein. Such localization can be determined using a spatially-resolved transcript amplicon readout mapping method, such as STARmap PLUS. STARmap PLUS is an image-based in situ RNA sequencing method described further in the Examples provided herein that utilizes paired primer and padlock probes (in together termed SNAIL probes) to convert a target RNA molecule into a DNA amplicon with a gene-unique code, which enables highly multiplexed RNA detection. STARmap PLUS is described in Wang, X. et al., “Three-dimensional intact-tissue sequencing of single-cell transcriptional states,” Science vol. 361 (2018); and in Hu Zeng, et al., “Integrative in situ mapping of single-cell transcriptional states and tissue histopathology in an Alzheimer's disease model,” bioRxiv (2022), the disclosures of which are incorporated herein by reference in their entireties for all purposes. The DNA amplicon is further chemically modified and embedded into a hydrogel to allow robust spatial readout of the unique code by multiple rounds of sequencing by ligation (SEDAL sequencing).

Accordingly, in various aspects the present disclosure provides methods and systems for characterizing cells and/or tissues. In embodiments, the tissue is an organ. In some cases, the tissues or cell forms part of the bone, central nervous system (e.g., brain or neuron), digestive tract, eye, muscle, immune cells, kidney, liver, cardiovascular system, and skin. In various instances, the cell is a neuron. In some cases, the cell is proliferating or non-proliferating.

In embodiments, a method for characterizing a cell or tissue involves introducing to the cell or tissue one or more polynucleotides or vectors provided herein, where each polynucleotide or vector encodes a unique barcode, unique RNA motif(s), unique epitope tag, and/or unique polypeptide that is orthogonal to one or more (e.g., all) other polynucleotides or vectors administered to the cell or tissue. This allows for the racRNA and/or polypeptide(s) expressed from one polynucleotide to be identified in a cell or tissue and distinguished from a racRNA and/or polypeptide(s) expressed from another polypeptide. Accordingly, the present disclosure provides methods for simultaneously selectively labeling multiple distinct cellular structures, components, and/or compartments using racRNAs of the disclosure.

In some cases, the systems, polynucleotides, and/or vectors of the disclosure may be used for integrative analysis of single-cell transcriptome and morphology, and/or RNA-barcode assisted morphological tracing for accurate cell segmentation in imaging-based spatial transcriptomic methods available to one of skill in the art.

In some cases, the methods of the present application may be used for cell cycle monitoring.

### Regulatory Sequences

In various aspects, the present disclosure provides a nucleotide sequence encoding a ribozyme-assisted circular RNA (racRNA) and/or polypeptides and associated regulatory sequences (e.g., a promoter described herein and other control sequences described herein). In embodiments, the polynucleotides further comprise 5′ and 3′ adeno-associated virus (AAV) inverted terminal repeats (ITRs). A coding sequence in certain embodiments is operatively linked to regulatory components in a manner which permits heterologous transcription, translation, and/or expression in a cell of a target tissue.

In some embodiments, the polynucleotides of the present invention comprise cis-acting 5′ and 3′ inverted terminal repeat (ITR) sequences described, e.g., by B. J. Carter, in “Handbook of Parvoviruses”, ed., P. Tijsser, CRC Press, pp. 155 168 (1990). The inverted terminal repeat (ITR) sequences can be about 50, 100, 125, 140, 145, or 150 bp in length. The ability to modify these inverted terminal repeat (ITR) sequences is within the skill of the art; see, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996). In various embodiments, a heterologous sequence comprised by a vector of the present invention and associated regulatory elements is flanked by 5′ and 3′ adeno-associated virus (AAV) inverted terminal repeat (ITR) sequences. The adeno-associated virus (AAV) inverted terminal repeat (ITR) sequences may be obtained from any known AAV, including, as non-limiting examples, AAV2, AAV7, AAV9, and AAV10.

In various embodiments, polynucleotides and vectors of the present invention also include expression control sequences operably linked to the heterologous gene in a manner which permits transcription, translation and/or expression of an racRNA and/or polypeptide encoded by a polynucleotide of the disclosure. Thus, the present invention in various aspects provides an expression cassette. As used herein, “operably linked” sequences include both expression control sequences that are contiguous with the gene of interest (i.e., act in trans) and expression control sequences that act in trans or at a distance to control the gene of interest. Expression control sequences include transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and sequences that enhance secretion of the encoded product. A great number of expression control sequences, including promoters which are native, constitutive, inducible and/or tissue-specific, are known in the art and are suitable for use in embodiments of the present invention. In some embodiments of the present invention a polyadenylation sequence can be inserted following a transcribed sequence encoding a polypeptide or racRNA molecule. In various embodiments, the polyadenylation sequence is inserted before a 3′ adeno-associated virus (AAV) inverted terminal repeat (ITR) sequence. Vectors of the present invention in various embodiments comprise an internal ribosome entry site (IRES). An IRES sequence is used to produce more than one polypeptide from a single gene transcript. An IRES sequence may be used to produce a protein that includes more than one polypeptide chain.

The precise nature of sequences needed for gene expression in host cells may vary between species, tissues or cell types. In some embodiments, vectors of the present invention comprise 5′ non-transcribed and 5′ non-translated sequences involved with the initiation of transcription and translation respectively of a heterologous gene, such as, to provide non-limiting examples, a TATA box, a capping sequence, a CAAT sequence, an enhancer elements, and the like. In various embodiments, a 5′ non-transcribed sequences can include a promoter region that includes a promoter sequence for transcriptional control of an operably joined gene. In some embodiments, vectors of the present invention include enhancer sequences or upstream activator sequences as desired. The polynucleotides and vectors of the disclosure may optionally include 5′ leader or signal sequences. The choice and design of an appropriate vector is within the ability and discretion of one of ordinary skill in the art.

Examples of suitable promoters include, but are not limited to the U6 promoter, the hSyn promoter, the retroviral Rous sarcoma virus (RSV) LTR promoter (optionally with the RSV enhancer), the cytomegalovirus (CMV) promoter (optionally with the CMV enhancer) (see, e.g., Boshart et al (1985) Cell, 41:521-530), the SV40 promoter, the dihydrofolate reductase promoter, the β-actin promoter (e.g., chicken β-actin promoter), the phosphoglycerol kinase (PGK) promoter, the EF1α promoter, the CBA promoter, UBC promoter, GUSB promoter, NSE promoter, Synapsin promoter, MeCP2 (methyl-CPG binding protein 2) promoter, GFAP; CBh promoter and the like. Exemplary promoters include, but are not limited to, the MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter; Niwa et al., Gene, 1991, 108(2):193-9) and the elongation factor 1-alpha promoter (EF1-alpha) promoter (Kim et al., Gene, 1990, 91(2):217-23 and Guo et al., Gene Ther., 1996, 3(9):802-10). In some embodiments, the promoter comprises a human 0-glucuronidase promoter or a cytomegalovirus enhancer linked to a chicken R-actin (CBA) promoter. The promoter can be a constitutive, inducible, or repressible promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (see, e.g., WO 98/10088); the ecdysone insect promoter (see, e.g., No et al, Proc. Natl. Acad. Sci. USA, 93:3346-3351 (1996)), the tetracycline-repressible system (see, e.g., Gossen et al, Proc. Natl. Acad. Sci. USA, 89:5547-5551 (1992)), the tetracycline-inducible system (see, e.g., Gossen et al, Science, 268:1766-1769 (1995), and Harvey et al, Curr. Opin. Chem. Biol., 2:512-518 (1998)), the RU486-inducible system (see, e.g., Wang et al, Nat. Biotech., 15:239-243 (1997) and Wang et al, Gene Ther., 4:432-441 (1997)) and the rapamycin-inducible system (see, e.g., Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only. In another embodiment, the native promoter for a heterologous gene comprised by the vector will be used. The native promoter may be preferred when it is desired that expression of the heterologous gene should mimic the native expression. The native promoter may be used when expression of the heterologous gene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., RNA Polymerase I, RNA Polymerase II, RNA Polymerase III). Exemplary promoters include, but are not limited to the SV40 early promoter, mouse mammary tumor virus long terminal repeat (“LTR”) promoter; adenovirus major late promoter (“Ad MLP”); a herpes simplex virus (“HSV”) promoter, a cytomegalovirus (“CMV”) promoter such as the CMV immediate early promoter region (“CMVIE”), a rous sarcoma virus (“RSV”) promoter, a human U6 small nuclear promoter (“U6”) (Miyagishi et al., “U6 promoter-driven siRNAs with four uridine 3′ overhangs efficiently suppress targeted gene expression in mammalian cells,” Nature Biotechnology 20:497-500 (2002), which is hereby incorporated by reference in its entirety), an enhanced U6 promoter (e.g., Xia et al., “An enhanced U6 promoter for synthesis of short hairpin RNA,” Nucleic Acids Res. 31(17):e100 (2003), which is hereby incorporated by reference in its entirety for all purposes), a human H1 promoter (“H1”), and the like.

Further examples of inducible promoters include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline, RNA polymerase, e.g., T7 RNA polymerase, an estrogen receptor, an estrogen receptor fusion, etc.

In one embodiment, the promoter is a prokaryotic promoter selected from the group consisting of T7, T3, SP6 RNA polymerase, and derivatives thereof. Additional suitable prokaryotic promoters include, without limitation, T71ac, araBAD, trp, lac, Ptac, and pL promoters.

In another embodiment, the promoter is a eukaryotic RNA polymerase I promoter, RNA polymerase III promoter, or a derivative thereof. Exemplary RNA polymerase II promoters include, without limitation, cytomegalovirus (“CMV”), phosphoglycerate kinase-1 (“PGK-1”), and elongation factor 1α (“EF1α”) promoters. In yet another embodiment, the promoter is a eukaryotic RNA polymerase III promoter selected from the group consisting of U6, H1, 56, 7SK, and derivatives thereof.

The RNA Polymerase promoter may be mammalian. Suitable mammalian promoters include, without limitation, human, murine, bovine, canine, feline, ovine, porcine, ursine, and simian promoters. In one embodiment, the RNA polymerase promoter sequence is a human promoter.

In some embodiments, the promoter expresses the heterologous gene in a brain cell and/or in a cell body disposed in the brain. A brain cell may refer to any brain cell known in the art, including without limitation a neuron (such as a sensory neuron, motor neuron, interneuron, dopaminergic neuron, medium spiny neuron, cholinergic neuron, GABAergic neuron, pyramidal neuron, etc.), a glial cell (such as microglia, macroglia, astrocytes, oligodendrocytes, ependymal cells, radial glia, etc.), a brain parenchyma cell, microglial cell, ependymal cell, and/or a Purkinje cell. In some embodiments, the promoter expresses the heterologous gene in a neuron. In some embodiments, the heterologous gene is exclusively expressed in neurons (e.g., expressed in a neuron and not expressed in other cells of the CNS, such as glial cells).

In some embodiments, vectors of the present invention comprise expression control sequences imparting tissue-specific gene expression capabilities. In some cases, the tissue-specific expression control sequences bind tissue-specific transcription factors that induce transcription in a tissue specific manner. Exemplary tissue-specific regulatory sequences include, but are not limited to, the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter; alpha-fetoprotein (AFP) promoter, bone osteocalcin promoter; bone sialoprotein promoter, CD2 promoter; immunoglobulin heavy chain promoter; T cell receptor α-chain promoter, neuronal such as neuron-specific enolase (NSE) promoter, neurofilament light-chain gene promoter, and the neuron-specific vgf gene promoter. In some embodiments, the expression control sequence allows for specific expression in the central nervous system (CNS) or a subset of one or more neurons or other CNS cells.

In some embodiments, one or more binding sites for one or more of miRNAs are incorporated in a heterologous gene of an adeno-associated virus vector, to inhibit the expression of the heterologous gene in one or more tissues of a subject harboring the heterologous gene, e.g., non-central nervous system (CNS) tissues. The skilled artisan will appreciate that miRNA binding sites may be selected to control the expression of a heterologous gene in a tissue-specific manner. In some embodiments, a binding site for a miRNA is in the 3′ UTR of the mRNA.

### Delivery of Polynucleotides

A cell of the invention, its progenitor, or its in vitro-derived progeny can contain a heterologous nucleotide sequence encoding genes to be expressed. Insertion of one or more pre-selected nucleotide molecules can be accomplished by homologous recombination or by viral integration into the host cell genome. The desired nucleotide molecule can also be incorporated into the cell, particularly into its nucleus, using a plasmid expression vector and a nuclear localization sequence. Methods for directing nucleotide molecules to the nucleus have been described in the art. The nucleotide molecules can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

Polynucleotides of the present disclosure may be delivered to a cell using any methods available in the art, such as through the use of a suitable vector (e.g., an adeno-associated virus vector) and/or through the use of electroporation. Methods for introducing polynucleotide sequences to a cell include those described, for example, in Kim and Eberwine, “Mammalian cell transfection: the present and the future,” Analytical and Bioanalytical Chemistry, 397: 3173-3178 (2010).

Administration of recombinant adeno-associated virus (rAAV) particles, nucleotide molecules, and/or vectors of the present invention to a subject may be by, for example, intramuscular injection or by administration into the bloodstream of the subject. Administration into the bloodstream may be by injection into a vein, an artery, or any other vascular conduit. In some embodiments, the recombinant adeno-associated virus (rAAV) particles, nucleotide molecules, and/or vectors are administered into the bloodstream by way of isolated limb perfusion, a technique well known in the surgical arts, the method essentially enabling the artisan to isolate a limb from the systemic circulation prior to administration. A variant of the isolated limb perfusion technique, described in U.S. Pat. No. 6,177,403, can also be employed by the skilled artisan to administer the recombinant adeno-associated virus (rAAV) particles, nucleotide molecules, and/or vectors into the vasculature of an isolated limb to potentially enhance transduction into muscle cells or tissue. Moreover, in certain instances, it may be desirable to deliver the virions to the central nervous system (CNS) of a subject. In various embodiments, by “CNS” is meant all cells and tissue of the brain and spinal cord of a vertebrate. Thus, the term can include, but is not limited to, neuronal cells, glial cells, astrocytes, cerebrospinal fluid (CSF), interstitial spaces, bone, cartilage and the like. Recombinant adeno-associated virus (rAAV) particles, nucleotide molecules, and/or vectors may be delivered directly to the central nervous system (CNS) or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

Calcium phosphate transfection can be used to introduce plasmid DNA containing a target gene or polynucleotide into a cell and is a standard method of DNA transfer to those of skill in the art. DEAE-dextran transfection, which is also known to those of skill in the art, may be preferred over calcium phosphate transfection where transient transfection is desired, as it is often more efficient. Since the cells of the present invention can be isolated cells, microinjection can be particularly effective for transferring genetic material into the cells. This method is advantageous because it provides delivery of the desired genetic material directly to the nucleus, avoiding both cytoplasmic and lysosomal degradation of the injected polynucleotide. Cells of the present invention can also be genetically modified using electroporation.

Liposomal delivery of nucleotide molecules to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPQ) can be added. Commercially available reagents for liposomal transfer include Lipofectin (Life Technologies). Lipofectin, for example, is a mixture of the cationic lipid N-[l-(2,3-dioleyloxy)propyl]-N—N—N-trimethyl ammonia chloride and DOPE. Liposomes can carry nucleotide molecules, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of nucleotide molecules into primary and established mammalian cell lines using lipopolyamine-coated nucleotide molecules can be used to introduce target DNA into the lymphatic endothelial progenitor cells described herein.

Naked plasmid DNA can be injected directly into a tissue comprising cells of the invention. This technique has been shown to be effective in transferring plasmid DNA to skeletal muscle tissue, where expression in mouse skeletal muscle has been observed for more than 19 months following a single intramuscular injection. More rapidly dividing cells take up naked plasmid DNA more efficiently. Therefore, it is advantageous to stimulate cell division prior to treatment with plasmid DNA. Microprojectile gene transfer can also be used to transfer nucleotide molecules into cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Transducing viral vectors (e.g., retroviral vectors (e.g., lentiviral vectors), alphaviral vectors (e.g., Sindbis vectors), adenoviral vectors, herpes virus vectors, and adeno-associated viral vectors) can be used for introducing a polynucleotide to a cell, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Peptide or polypeptide transfection is another method that can be used to genetically alter lymphatic endothelial progenitor cells of the invention and their progeny. Peptides such as Pep-1 (commercially available as Chariot), as well as other polypeptide transduction domains, can quickly and efficiently transport biologically active polypeptides, peptides, antibodies, and nucleic acids directly into cells, with an efficiency of about 60% to about 95% (Morris, M. C. et al, (2001) Nat. Biotech. 19: 1173-1176).

### Adeno-Associated Virus (AAV)

AAV is a small (25 nm), nonenveloped virus that contains a linear single-stranded DNA genome packaged into the viral capsid. AAV belongs to the family Parvoviridae and is of the genus Dependovirus. Productive infection by AAV occurs only in the presence of either an adenovirus or herpesvirus helper virus. In the absence of helper virus, AAV (serotype 2) can establish latency after transduction into a cell by specific but rare integration into chromosome 19q13.4. Accordingly, AAV is the only mammalian DNA virus known to be capable of site-specific integration. (Daya, S. and Berns, K I., 2008, Clin. Microbiol. Rev., 21(4):583-593). There are two stages to the AAV life cycle after successful infection: a lytic stage and a lysogenic stage. In the presence of adenovirus or herpesvirus helper virus, the lytic stage persists. During this period, AAV undergoes productive infection characterized by genome replication, viral gene expression, and virion production. The adenoviral genes that provide helper functions for AAV gene expression include E1a, E1b, E2a, E4, and VA RNA. While adenovirus and herpesvirus provide different sets of genes for helper function, they both regulate cellular gene expression and provide a permissive intracellular milieu for a productive AAV infection. Herpesvirus aids in AAV gene expression by providing viral DNA polymerase and helicase as well as the early functions necessary for HSV transcription.

In the absence of adenovirus or herpesvirus, AAV replication is limited; viral gene expression is repressed; and the AAV genome can establish latency by integrating into a 4-kb region on chromosome 19 (q13.4), called AAVS1. The AAVS1 locus is near several muscle-specific genes, TNNT1 and TNNI3. The AAVS1 region itself is an upstream part of the gene MBS85 whose product has been shown to be involved in actin organization. Tissue culture experiments suggest that the AAVS1 locus is a safe integration site.

AAV has attracted considerable interest as a vector for use in polynucleotide delivery to subjects due to a number of desirable features. Chief amongst these is the virus's lack of pathogenicity. AAV can also infect non-dividing cells and has the ability to stably integrate into the host cell genome at a specific site (designated AAVS1) in the human chromosome 19. A desired gene together with a promoter to drive transcription of the gene can be inserted between the inverted terminal repeats (ITRs) that aid in concatemer formation in the nucleus after the single-stranded vector DNA is converted by host cell DNA polymerase complexes into double-stranded DNA. Non-integrating AAV-based polynucleotide therapy vectors typically form episomal concatemers in the host cell nucleus. In non-dividing cells, these concatemers remain intact for the life of the host cell. In dividing cells, non-integrating AAV DNA is lost through cell division, since the episomal DNA is not replicated along with the host cell DNA. As a viral vector, AAV can be used to deliver myriad polynucleotides to a subject and/or a population of cells or different cell types.

Recombinant AAV (rAAV) for Delivery of Polynucleotides

The disclosure provides for recombinant adeno-associated virus (rAAV) particles (alternatively, “AAV vectors”) containing the polynucleotides provided herein. In embodiments, the polynucleotides are rAAV genomes.

AAVs are well suited for use as vectors and vehicles for gene transfer to cells. AAVs provide safe, long-term expression in a cell (e.g., a nerve cell). AAV vectors have been highly successful in fulfilling all of the features desired for a delivery vehicle, such as the ability to attach to and enter the target cell, successful transfer to the nucleus, the ability to be expressed in the nucleus for a sustained period of time, and a general lack of pathogenicity and toxicity. Recombinant AAV (rAAV) is advantageous as a delivery vector, particularly for delivery to the central nervous system, as it is focally injectable; it exhibits stable expression over time; and it is both non-pathogenic and non-integrative into the genome of the cell into which it is transduced. Twelve human serotypes of AAV (AAV serotype 1 (AAV-1) to AAV-12) and more than 100 serotypes from nonhuman primates have been reported to date. (Daya, S. and Berns, K. I., 2008, Clin. Microbiol. Rev., 21(4):583-593). In addition, rAAV has been approved by the FDA for use as a vector in at least 38 protocols for several different human clinical trials. AAV's lack of pathogenicity, persistence and its many available serotypes have increased the potential of the virus as a delivery vehicle for a gene therapy application in accordance with the described compositions and methods.

In embodiments, the polynucleotides can be encapsidated by AAV-PHP.B (see, e.g., Deverman, et al. “Cre-dependent selection yields AAV variants for widespread gene transfer to the adult brain,” Nat Biotechnol. 2016 February; 34(2):204-209. PMCID: PMC5088052, the disclosure of which is incorporated herein by reference in its entirety for all purposes), an AAV-PHP.eB (described in Deverman B E, Pravdo P L, Simpson B P, Kumar S R, Chan K Y, Banerjee A, Wu W-L, Yang B, Huber N, Pasca S P, Gradinaru V. Cre-dependent selection yields AAV variants for widespread gene transfer to the adult brain. Nat Biotechnol. 2016 February; 34(2):204-209. PMCID: PMC5088052; and Chan K Y, Jang M J, Yoo B B, Greenbaum A, Ravi N, Wu W-L, Sinchez-Guardado L, Lois C, Mazmanian S K, Deverman B E, Gradinaru V. Engineered AAVs for efficient noninvasive gene delivery to the central and peripheral nervous systems. Nat Neurosci. 2017 August; 20(8):1172-1179. PMCID: PMC5529245), AAVF (described in Hanlon K S, Meltzer J C, Buzhdygan T, Cheng M J, Sena-Esteves M, Bennett R E, Sullivan T P, Razmpour R, Gong Y, Ng C, Nammour J, Maiz D, Dujardin S, Ramirez S H, Hudry E, Maguire C A. Selection of an Efficient AAV Vector for Robust CNS Transgene Expression. Mol Ther Methods Clin Dev. 2019 Dec. 13; 15:320-332. PMCID: PMC6881693, the disclosure of which is incorporated herein by reference in its entirety for all purposes), AAV-PHP.B4-B8, AAV-PHP.C1-C3 (Kumar, S. R. et al. Multiplexed Cre-dependent selection yields systemic AAVs for targeting distinct brain cell types. Nat Methods 17, 541-550 (2020), 9P31) or other capsids with similar properties (Nonnenmacher, M. et al. Rapid Evolution of Blood-Brain Barrier-Penetrating AAV Capsids by RNA-Driven Biopanning. Mol Ther-Methods Clin Dev (2020) doi:10.1016/j.omtm.2020.12.006), or CAP-B10 or CAP-B22 (Goertsen, D. et al. AAV capsid variants with brain-wide transgene expression and decreased liver targeting after intravenous delivery in mouse and marmoset. Nat Neurosci 1-10 (2021) doi:10.1038/s41593-021-00969-4). Further non-limiting examples of AAV capsids suitable for encapsidation of polynucleotides of the disclosure include those described in PCT/US2019/044796, PCT/US2020/027708, PCT/US2020/044487, or PCT/US2020/015972, the disclosures of each of which are incorporated herein by reference in their entireties for all purposes.

In some instances, the polynucleotide is encapsidated by a blood-brain barrier crossing AAV capsid. In various embodiments, the methods of the invention involve delivering one or more polynucleotides provided herein broadly to a host using an intravenously administered AAV capsid encapsidating the polynucleotides. In some cases, the polynucleotides are encapsidated by and delivered to a cell using the AAV-PHP.eB capsid. In other embodiments, the polynucleotides are encapsidated in a capsid suitable for efficient, broad expression after direct delivery into the brain or other target organ.

In some instances, the polynucleotide is encapsidated by an AAV vector capable of retrograde transport of a polynucleotide payload to the nucleus of a neuron (e.g., an AAVretro AAV vector, such as those described in Tervo, et al. “A designer AAV variant permits efficient retrograde access to projection neurons,” Neuron, 92:372-382 (2016), the disclosure of which is incorporated herein by reference in its entirety for all purposes).

Recombinant AAV (rAAV) vectors have been constructed with genomes that do not encode the replication (Rep) proteins and that lack the cis-active, 38 base pair integration efficiency element (IEE), which is required for frequent site-specific integration. The inverted terminal repeats (ITRs) are retained because they are the cis signals required for packaging. Thus, current polynucleotides delivered using AAV capsids (i.e., as AAV vectors) persist primarily as extrachromosomal elements.

AAV-2-based rAAV vectors can transduce muscle, liver, brain, retina, and lungs, requiring several days to weeks for optimal expression. The efficiency of rAAV transduction is dependent on the efficiency at each step of AAV infection, i.e., virus binding, entry, trafficking, nuclear entry, uncoating, and second-strand synthesis.

Recombinant AAV vectors can be made using standard and practiced techniques in the art and employing commercially available reagents. In some embodiments, plasmid vectors may encode all or some of the well-known replication (rep), capsid (cap) and adeno-helper components. The rep component comprises four overlapping genes encoding Rep proteins required for the AAV life cycle (e.g., Rep78, Rep68, Rep52 and Rep40). The cap component comprises overlapping nucleotide sequences of capsid proteins VP1, VP2 and VP3, which interact together to form a capsid of an icosahedral symmetry. A second plasmid that encodes helper components and provides helper function for the AAV vector may also be co-transfected into cells. Non-limiting examples of helper components include the adenoviral genes E2A, E4orf6, and VA RNAs for viral replication.

In an embodiment, a method of making rAAVs for the products, compositions, and uses described herein involves culturing cells that comprise an rAAV polynucleotide expression vector (e.g., a polynucleotide containing a polynucleotide); culturing the cells to allow for expression of the polynucleotides to produce the rAAVs within the cell and separating or isolating the rAAVs from cells in the cell culture and/or from the cell culture medium. Such methods are known and practiced by those having skill in the art. The rAAVs can be purified from the cells and cell culture medium to any desired degree of purity using conventional techniques.

Recombinant AAV vectors, which have a genome of small size (about 5 kb), can be engineered to package and contain larger genomes (transgenes), e.g., those that are greater than 4.7 kb. By way of example, two approaches developed to package larger amounts of genetic material (genes, polynucleotides, nucleic acid) include split AAV vectors and fragment AAV (fAAV) genome reassembly (Hirsch, M. L. et al., 2010, Mol Ther 18(1):6-8; Hirsch, M. L. et al., 2016, Methods Mol Biol, 1382:21-39).

An advantage and benefit of the vectors, compositions and methods described herein is their use in the delivery of circular RNAs to the cytoplasm of a cell and/or their selective delivery to other compartments of the cell. In embodiments, the vectors may be used to characterize a cell or tissue.

### Cell-Specific AAV Capsids

The rational design of AAV vectors that display selective tissue/organ targeting has broadened the applications of AAV as vector/vehicle for polynucleotide delivery to cells. Both direct and indirect targeting approaches have been used to enhance AAV vector cell targeting specificity and retargeting. By way of example, in direct targeting, AAV vector targeting to certain cell types is mediated by small peptides or ligands that have been directly inserted into the viral capsid sequence. This approach has been successfully employed to target endothelial cells. Direct targeting requires detailed knowledge of the capsid structure such that peptides or ligands are positioned at sites that are exposed to the capsid surface; the insertion does not significantly affect capsid structure and assembly; and the native tropism is ablated to maximize targeting to a specific cell type. In indirect targeting, AAV vector targeting is mediated by an associating molecule that interacts with both the viral surface and the specific cell surface receptor. Such associating molecules for AAV vectors may include bispecific antibodies and biotin. The advantages of indirect targeting are that different adaptors can be coupled to the capsid without resulting in significant changes in the capsid structure, and the native tropism can be easily ablated. A disadvantage of using adaptors for targeting involves a potential for decreased stability of the capsid-adaptor complex in vivo.

In addition, AAV vectors may be produced that comprise capsids that allow for the increased transduction of cells and gene transfer to the central nervous system and the brain via the vasculature (Chan, K. Y. et al., 2017, Nat. Neurosci., 20(8):1172-1179). Such vectors facilitate robust transduction of neuronal cells, including interneurons. In embodiments, AAV vectors contain an AAVF, AAV-PHP.B4, AAV-PHP.B5, AAV-PHP.C1, 9P31, or an AAV-PHP.eB capsid.

### Delivery of Recombinant Adeno-Associated Viral Vectors

For direct delivery to the brain, rAAV vectors may be administered by open neurosurgical procedure or by focal injection in order to bypass the blood-brain barrier, to temporally and spatially restrict transgene expression, and to target specific areas of the brain, e.g., interneuron cells and brain tissue comprising these cells.

Systemic rAAV delivery (by intravenous injection) provides a non-invasive alternative for broad gene delivery to the nervous system. Several groups have developed rAAV capsids that enhance gene transfer to the CNS and certain tissues and cell populations after intravenous delivery. By way of example, AAV-AS capsid18 utilizes a polyalanine N-terminal extension to the AAV9.4719 VP2 capsid protein to provide higher neuronal transduction, particularly in the striatum. The AAV-BR1 capsid20, based on AAV2, may be useful for more efficient and selective transduction of brain endothelial cells. Another AAV capsid, AAV-PHP.B, comprises a capsid that transduces the majority of neurons and astrocytes across many regions of the adult mouse brain and spinal cord after intravenous injection.

Other modes of rAAV vector administration may include lipid-mediated vector delivery, hydrodynamic delivery, and a gene gun.

The virus vectors and compositions thereof as described herein may be used to characterize the tropism of an AAV vector or library of AAV vectors in vivo. In embodiments, such characterization involves cell-type-resolved quantification of AAV vector tropisms.

### RNA Editing

Guide RNA engineering has been an important route to increase the efficiency and versatility of CRISPR-based and ADAR-editing-based technologies, where “ADAR” refers to “adenosine deaminases that act on RNA.” Methods for editing RNA in a cell using an ADAR are known to one of skill in the art and described, for example, in Brenda Bass, “RNA Editing by Adenosine Deaminases that Act on RNA,” Annu Rev Biochem, 71: 817-846 (2002), the disclosure of which is incorporated herein by reference in its entirety for all purposes. In embodiments, RNA is edited in a cell by contacting the cell with an ADAR or polynucleotide encoding the same, and the guide RNA used to target an ADAR is provided to the ADAR as a segment of a ribozyme-assisted circular RNA (racRNA) of the present disclosure. In embodiments, the increased stability of the guide RNA presented as a segment of a racRNA enhances ADAR-mediated RNA editing in vitro and in vivo. In embodiments, a racRNA expressed in a cell in combination with circular RNA shuttling or exporting polypeptides provided herein is used to achieve cell-type-specific RNA editing by placing expression of the racRNA and/or shuttling and/or exporting polypeptides under the control of a cell-type specific promoter.

### RNA Control

The CRISPR-Cas-inspired RNA targeting system (CIRTS), is a Cas13-inspired system that uses a defined protein-RNA interaction to display a gRNA sequence to deliver protein cargoes to a target RNA for programmable RNA control (see Condrat C E, et al., “miRNAs as Biomarkers in Disease: Latest Findings Regarding Their Role in Diagnosis and Prognosis. Cells 2020; 9. doi:10.3390/cells9020276, the disclosure of which is incorporated herein by reference in its entirety for all purposes). In embodiments, the guide RNA in this system is delivered to a cell as a segment of a racRNA of the disclosure to increase guide stability and enhance the presence of the guide RNA in the cytoplasm where RNA translation and degradation actively occur, together improving CIRTS efficiency.

### RNA Sponges

In embodiments, ribozyme-assisted circular RNAs (racRNAs) of the disclosure may be administered to a subject as therapeutic sponges and nuclear sequesters of toxic RNAs in associated with a disease or disorder. For example, the ribozyme-assisted circular RNA may comprise an RNA segment complementary to a pathogenic RNA molecule in a cell. In embodiments, the circular RNAs are expressed and/or localized in the nucleus or cytoplasm and act as molecular sponges (Panda A C., Circular RNAs Act as miRNA Sponges, Adv Exp Med Biol 2018; 1087: 67-79). In embodiments the molecular sponges sequester pathogenic or toxic nucleotide molecules in the nucleus and diminish their pathological roles. Non-limiting examples of toxic RNAs include (1) disease-causing mRNAs that carry mutations that misregulate splicing or cause protein mutations (e.g., gain-of-function mutation on DMPK in type 1 Myotonic dystrophy (DM1) and gain-of-function mutation on JPH3 in Huntington's disease-like 2 (HDL2)); and (2) overexpressed aberrant miRNAs in diseases (e.g., miR-10b in metastatic breast cancer).

### Molecular Identifiers

For a convenient detection of a polynucleotide, the polynucleotide can be coupled to a molecular identifier (e.g., a unique molecular identifier, such as a barcode). Molecular identifiers suitable for use in the present invention include any agent detectable by photochemical, biochemical, spectroscopic, immunochemical, electrical, optical or chemical means. In some embodiments, a probe described herein is linked to a nucleotide sequence (e.g., a barcode) that is used for molecular identification.

A wide variety of appropriate molecular identifiers are known in the art, which include fluorescent or chemiluminescent labels, radioactive isotope labels, enzymatic or other ligands. The molecular identifier can be a fluorescent label (e.g., a fluorescent protein) or an enzyme tag, such as digoxigenin, β-galactosidase, urease, alkaline phosphatase or peroxidase, avidin/biotin complex.

Radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels may be detected by visualizing a colored label.

Specific non-limiting examples of molecular identifiers include radioisotopes, such as 32P, 14C, 125I, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the invention.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent bar code.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag (e.g., a barcode) may be an oligonucleotide that may be detected by sequencing of its nucleotide sequence and/or by detecting non-nucleic acid detectable moieties to which it may be attached.

In embodiments, the molecular identifier is a microparticles including as non-limiting examples quantum dots (Empodocles, et al., Nature 399:126-130, 1999), gold nanoparticles (Reichert et al., Anal. Chem. 72:6025-6029, 2000).

### Barcoding

In one embodiment of the disclosure, a plasmid barcoding system was developed to generate microgram amounts of high-quality, circularized plasmid. This system, i.e., the “barcoding plasmid pipeline,” may introduce barcodes into any position of any plasmid of interest. An embodiment begins with a non-barcoded plasmid used as a template for PCR reactions in which random DNA sequences (barcodes) as well as shared restriction site cassettes are introduced through forward and reverse primers. Hundreds of micrograms of linear, double-stranded PCR amplicons encompassed the entire plasmid sequence with barcodes introduced on each terminal end of the amplified molecules. A further embodiment comprises circularizing the linear amplicons with a series of enzymes (such as in a single-tube), fusing the two terminal barcodes into a single barcode cassette, and eliminating any residual non-barcoded template plasmid.

### Compositions

Provided also are compositions (e.g., pharmaceutical compositions) containing racRNAs, vectors, polypeptides, and/or polynucleotides of the disclosure, and for use in the methods of the disclosure. In embodiments, the composition is a pharmaceutical composition for use in treating a disease or disorder. In some instances, a composition of the disclosure is used in a diagnostic method (e.g., to detect a marker associated with a disease). In an embodiment, the compositions contain a cell, polynucleotide, vector, or polypeptide provided herein. In some cases, the composition contains a polynucleotide or racRNA as described herein and an acceptable carrier, excipient, or diluent.

The agents of the disclosure (e.g., polynucleotides, polypeptides, vectors, and/or cells) may be contained in any appropriate amount in any suitable carrier substance, and is/are present in some cases in an amount of 0.01-95% by weight of the total weight of the composition. A pharmaceutical composition may be provided in a form that is suitable for a parenteral (e.g., subcutaneous, intravenous, intramuscular, or intraperitoneal) administration route, such that the agent, such as a vector or cell described herein, is systemically delivered.

The compositions of the present invention can be prepared in accordance with known techniques. See, e.g., Remington, The Science And Practice of Pharmacy (21st ed. 2005). In some embodiments, an agent of the disclosure is present in a reconstitutable dry composition (e.g., a lyophilized composition or powder). In embodiments, an agent is admixed with a suitable carrier prior to administration or storage, and in some embodiments, the composition further comprises an acceptable carrier (e.g., a pharmaceutically acceptable carrier). Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Carriers can include agents that can stabilize, optimize or otherwise alter the form, consistency, viscosity, pH, pharmacokinetics, or solubility of a composition. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Compositions of the disclosure can contain one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Compositions can also contain one or more osmotic modulating agents, i.e., a compound that modulates the osmotic properties (e.g., tonicity, osmolality, and/or osmotic pressure) of the formulation to a level that is acceptable, for example, to the blood stream and blood cells of recipient subjects. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

The skilled artisan can readily determine the number of cells and amount of optional additives, vehicles, and/or carriers in compositions and to be administered in methods of the invention. Of course, for any composition to be administered to an animal or human, and for any particular method of administration, it is preferred to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein, and the time for sequential administrations can be ascertained without undue experimentation.

In some embodiments, the composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. The pharmaceutical composition may be administered systemically.

The composition may be in the form of a solution, a suspension, an emulsion, an infusion device, or a delivery device for implantation, or it may be presented as a dry powder to be reconstituted with water or another suitable vehicle before use. Apart from the agent (e.g., racRNAs, polynucleotides, or polypeptides provided herein), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition are formulated for intravenous delivery. The compositions according to the described embodiments may be in a form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Ringer's solution, isotonic sodium chloride solution and dextrose solution. The aqueous formulation may also contain one or more preservatives (e.g., methyl, ethyl, or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Except insofar as any conventional excipient medium is incompatible with a substance or its derivatives, such as by producing any undesirable biological effect or otherwise interacting in a deleterious manner with any other component(s) of the composition, its use is contemplated to be within the scope of this disclosure.

In some embodiments, compositions in accordance with the present disclosure can be used for treatment of any of a variety of diseases, disorders, and/or conditions.

### Treatments

The compositions, polynucleotides, racRNAs, cells, and/or polypeptides provided herein can be used for treating a subject for a disease or disorder. Generally, the methods provided herein include administering a therapeutically effective amount of an agent as provided herein, to a subject who is in need of, or who has been determined to be in need of, such treatment.

A further aspect of the present invention relates to a treatment method. This treatment method involves contacting a cell with a racRNA molecule of the present invention under conditions effective to express the molecule to treat the cell.

According to one embodiment, this and other treatment methods described herein are effective to treat a cell, e.g., a cell under a stress or disease condition. Exemplary cell stress conditions may include, without limitation, exposure to a toxin; exposure to chemotherapeutic agents, irradiation, or environmental genotoxic agents such as polycyclic hydrocarbons or ultraviolet (UV) light; exposure of cells to conditions such as glucose starvation, inhibition of protein glycosylation, disturbance of Ca²⁺ homeostasis and oxygen; exposure to elevated temperatures, oxidative stress, or heavy metals; and exposures to a pathological disease state (e.g., diabetes, Parkinson's disease, cardiovascular disease (e.g., myocardial infarction, end-stage heart failure, arrhythmogenic right ventricular dysplasia, and Adriamycin-induced cardiomyopathy), and various cancers (Fulda et al., “Cellular Stress Responses: Cell Survival and Cell Death,” Int. J Cell Biol. (2010), which is hereby incorporated by reference in its entirety).

Various embodiments of the racRNA molecules of the present invention are described above and apply in carrying out this and other treatment methods described herein.

In some embodiments, contacting a cell with an RNA molecule of the present invention involves introducing an RNA molecule into a cell. Suitable methods of introducing RNA molecules into cells are well known in the art and include, but are not limited to, the use of transfection reagents, electroporation, microinjection, or via viruses.

The cell may be a eukaryotic cell. Exemplary eukaryotic cells include a yeast cell, an insect cell, a fungal cell, a plant cell, and an animal cell (e.g., a mammalian cell). Suitable mammalian cells include, for example without limitation, human, non-human primate, cat, dog, sheep, goat, cow, horse, pig, rabbit, and rodent cells.

In another embodiment, the RNA molecule of the present invention may be isolated or present in in vitro conditions for extracellular expression and/or processing. According to this embodiment, the RNA molecule is contacted by an RNA ligase (e.g., RtcB) in vitro, purified, circularized, and then the circularized RNA molecule is administered to a cell or subject for treatment.

Treating cells also includes treating the organism in which the cells reside. Thus, by this and the other treatment methods of the present invention, it is contemplated that treatment of a cell includes treatment of a subject in which the cell resides.

In one embodiment of carrying out this method of the present invention, the vector encodes racRNA that contains a polynucleotide of interest that has a therapeutic effect. The polynucleotide may be endogenous or heterologous to the cell. The polynucleotide may serve to up-regulate or down-regulated expression of a protein in a disease state, a stress state, or during a pathogen infection in a cell.

An effective amount of an agent (e.g., a racRNA) can be administered in one or more administrations, applications or dosages. A therapeutically effective amount of a therapeutic compound or agent (i.e., an effective dosage) depends on the therapeutic compounds or agents selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic agents provided herein can include a single treatment or a series of treatments.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents which exhibit high therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from cell culture assays and animal studies can be used in formulating a range of dosage for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test agent which achieves a half-maximal inhibition of symptoms) as determined in cell culture. Such information can be used to determine useful doses more accurately in humans. Levels in plasma may be measured, for example, by high performance liquid chromatography.

Dosages and desired drug concentration of pharmaceutical compositions of the present disclosure may vary depending on the particular use envisioned. The determination of the appropriate dosage or route of administration (e.g., oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes) is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg. about 100 μg/kg, about 300 μg/kg, about 1 mg/kg. or about 2 mg/kg) may be used. In certain embodiments, dosing frequency is three times per day, twice per day, once per day. once every other day. once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Methods for characterizing the efficacy of a treatment for a neoplasia are well known in the art (e.g., computerized tomography (CT) scan, bone scan, magnetic resonance imaging (MRI), position emission tomography (PET) scan, ultrasound X-ray, biopsy, etc.).

### Implementation in Hardware

In various aspects, the methods described herein are conducted with the aid of a computer-based system configured to execute machine-readable instructions, which, when executed by a processor of the system causes the system to perform steps including determining the identity, size, nucleotide sequence or other measurable characteristics of the amplicons produced in the method of the invention. One or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When using a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, Pascal, Basic, Fortran, Cobol, Pert, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

### Kits

The invention provides kits for use in the methods of the disclosure. The agents described herein may, in some embodiments, be assembled into research or diagnostic kits to facilitate their use in diagnostic or research applications. In certain embodiments agents in a kit may be in compositions suitable for a particular application and for a method of administration of the agents. Kits for research purposes may contain the components in appropriate concentrations or quantities for running various experiments (e.g., cell and/or tissue characterization).

Kits may include ampules or aliquots of compositions of the present invention. Kits may also contain devices to be used in administering the compositions. In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding compositions of the disclosure.

The kit may be designed to facilitate use of the methods described herein. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or another suitable solvent), which may or may not be provided with the kit.

The kit may contain any one or more of the components described herein in one or more containers. As an example, in one embodiment, the kit may include instructions for mixing one or more components of the kit and/or isolating and mixing a sample and administering to a subject. The kit may include a container housing agents described herein. The agents may be in the form of a liquid, gel or solid (powder). The agents may be prepared sterilely, packaged in syringe and shipped refrigerated. A second container may comprise other agents prepared sterilely. Alternatively, the kit may include agents premixed and shipped in a syringe, vial, tube, or other container. The kit may have one or more or all of the components useful to administer the agents to a subject, such as a syringe, topical application devices, or intravenous needle tubing and bag.

If desired an agent of the invention is provided together with instructions for administering an agent of the present invention to a subject. The instructions will generally include information about the use of the composition in a method of the disclosure. The instructions may be printed directly on the container (when present), provided on a transportable storage medium, stored on a remote server, or provided as a label applied to the container, or as a separate sheet, pamphlet, card, or folder supplied in or with the container. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Hybrid Readily Exported Cis RNA Sequence Elements with Synthetic RNA

Circular RNAs lack exposed 5′- and 3′-ends and are thus resistant to exonuclease degradation. Its ultra-stability inside cells makes it an ideal vector for exogenous RNA sequences or barcodes. To this end, the Tornado expression system (Litke J L, Jaffrey S R., Highly efficient expression of circular RNA aptamers in cells using autocatalytic transcripts, Nat Biotechnol 2019; 37: 667-675) was utilized to produce circular RNAs with a barcode sequence under a human U6 promoter (FIG. 2A). In the tornado expression system, RNA sequences of interest are flanked by ribozymes at both ends. Self-cleavage of the two ribozymes gives rise to reactive ends that can be ligated by endogenous tRNA processing ligase, yielding racRNA (nibozyme-assisted circular RNA). The barcode on the circular RNA allowed specific and sensitive detection using STARmap (see Wang X, et al. “Three-dimensional intact-tissue sequencing of single-cell transcriptional states,” Science 2018; 361. doi:10.1126/science.aat5691; and Hu Zeng, et al. “Integrative in situ mapping of single-cell transcriptional states and tissue histopathology in an Alzheimer's disease model,” bioRxiv 2022. doi:10.1101/2022.01.14.476072, the disclosures of which are incorporated herein by reference in their entireties for all purposes). The circular RNA also contains a PP7 hairpin to be recognized by the PP7 (Chao J A, et al. “Structural basis for the coevolution of a viral RNA-protein complex,” Nat Struct Mol Biol 15:103-105 (2008), the disclosure of which is incorporated herein by reference in its entirety for all purposes) coat protein (PP7cp), thus named racPP7 (FIG. 2A). The hCTE and BC1 RNA sequences were inserted into the circular RNA expression system, resulting in racPP7-hCTE and racBC1 (FIGS. 2B-2C). in vitro confirmation that the racPP7, racPP7-hCTE, and racBC1 were indeed circularized, resistant to RNase R digestion (FIG. 2D) was established. Besides the PP7 hairpin, the racRNA expression backbone was also confirmed to work for RNA hairpins, including BoxB and MS2 (FIG. 2D).

### Example 2: Engineer Nuclear-Cytoplasmic Shuttling Protein Binding Partners for the Synthetic RNA

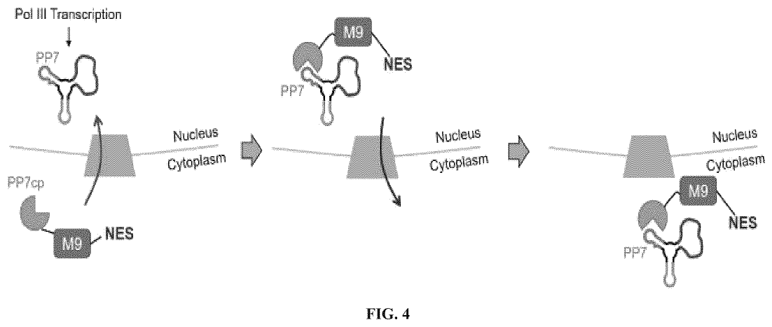

To prepare a polypeptide for shutting mRNA out of the nucleus, PP7cp was fused to an M9 tag to allow for PP7-containing racRNAs to be shuttled out of the nuclei with high turnovers (FIG. 4). Additionally, another nuclear export signal (NES) sequence was added to the fusion protein to enhance export functionality.

### Example 3: Demonstration in Proliferating Cell Cultures

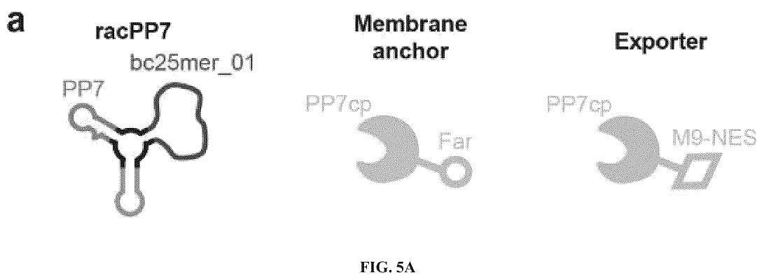

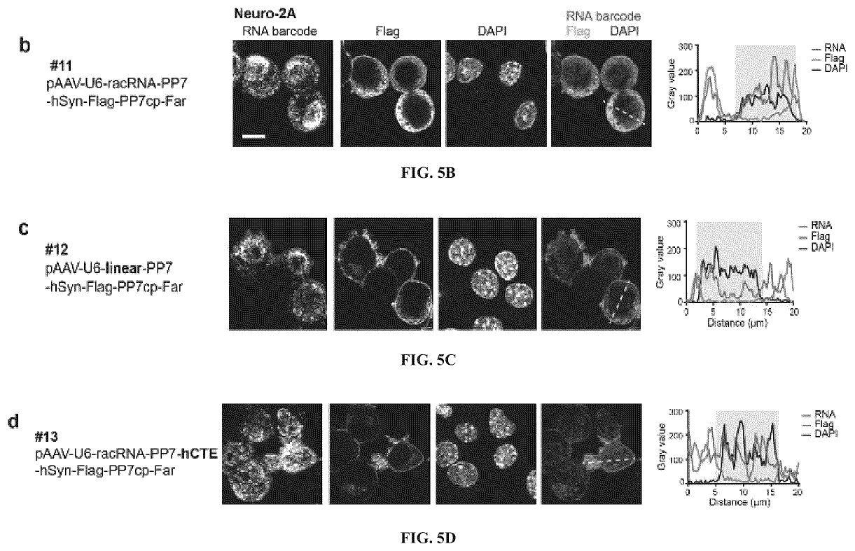

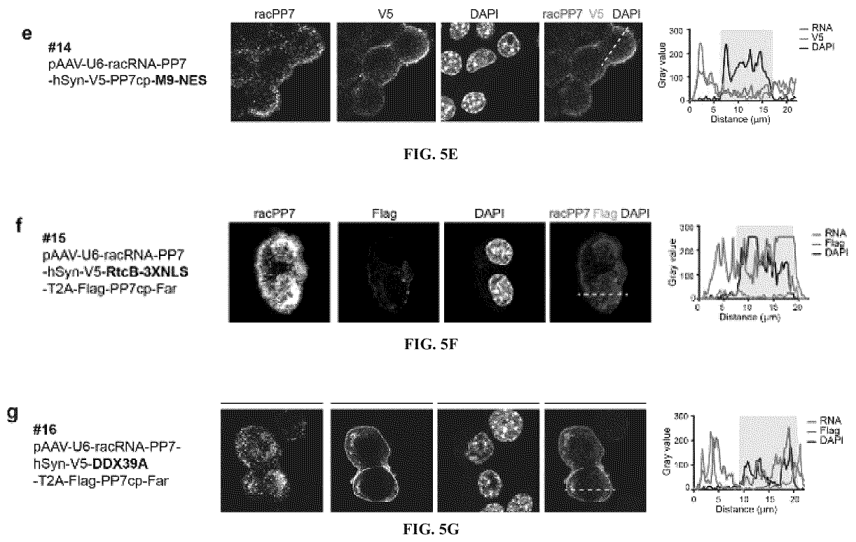

Strategies in proliferating cell cultures were tested using Neuro-2A cells as an example (FIGS. 5A-5G). The cells were transfected with plasmids of different RNA export designs and RNA barcode distribution was detected inside cells by STARmap in 24 hours. A PP7cp was designed to be tagged with a famesylation motif for lipid modification and thus membrane anchoring (PP7cp-Far) to facilitate the visualization of nuclear-exported RNA barcodes.

Observations were that (1) without export-facilitating elements, a decent amount of the circular RNA barcodes remained in the cell nucleus (FIG. 5B): while the PP7cp-Far protein itself correctly localizes at the membrane, the racPP7 was restricted to the nuclei; (2) the cis-element terminal helix and the trans-elements RtcB and DDX39A (for short circular RNA<400 nt, racPP7, ˜220 nt) showed limited effects in RNA barcode exporting (FIGS. 5C, 5F, and 5G); (3) Among all the designed tests, the cis-element hCTE and the trans-element M9-NES (strategy 3) showed the largest improvement in RNA barcode export (FIGS. 5C-5D).

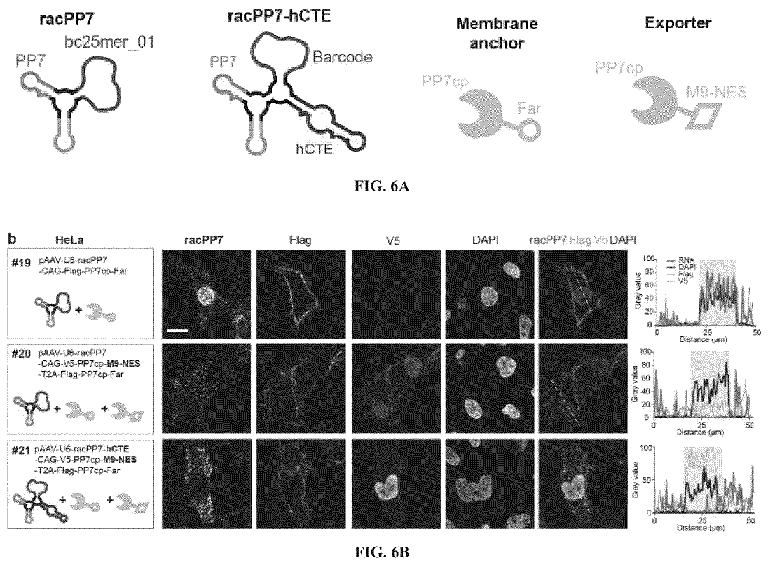

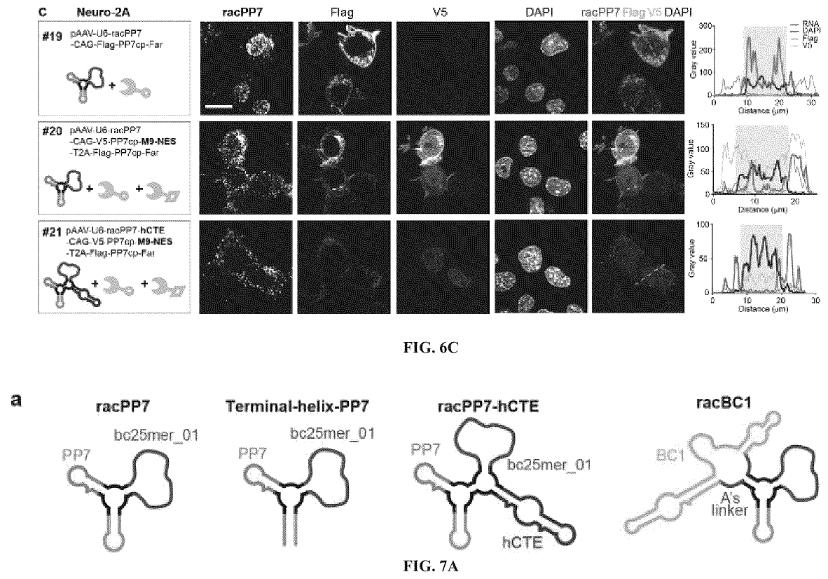

Next, constructs were tested that combined the cis- and trans-elements in both human (HeLa) and mouse (Neuro-2A) proliferating cell cultures (FIG. 6A). While racPP7 by itself largely remained in the nucleus, co-expressing the exporter PP7cp-M9-NES and the membrane anchor PP7cp-Far greatly removed the STARmap barcode amplicons from the nuclei (FIGS. 6B-6C). Supplementing the racPP7 with the hCTE further improved nuclear export in Neuro-2A cells (FIG. 6C).

Note that RNA localization in dividing cells is confounded by cell proliferation, wherein the prophase cell nucleus dissolves and nuclear RNA enters the cytoplasm. Therefore, non-dividing primary cell cultures were used next to obtain a more conclusive examination of the export strategies.

### Example 4: Demonstration in Primary Neuronal Cell Cultures

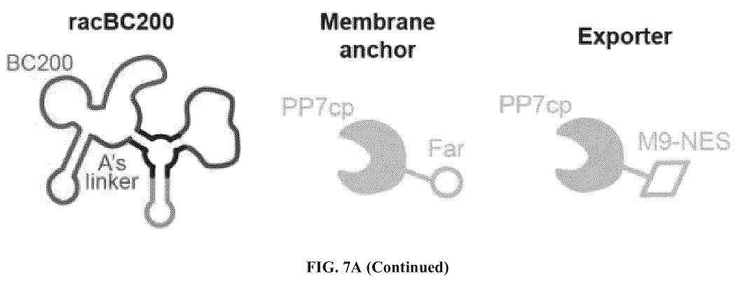

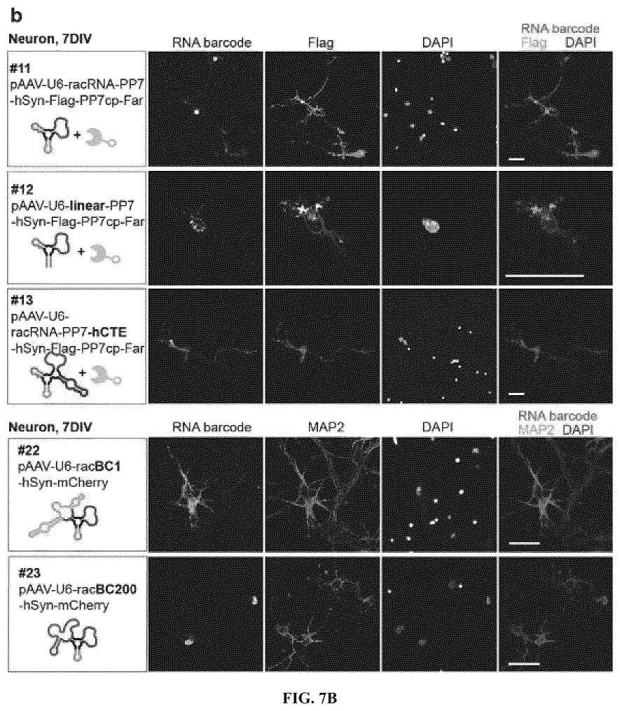

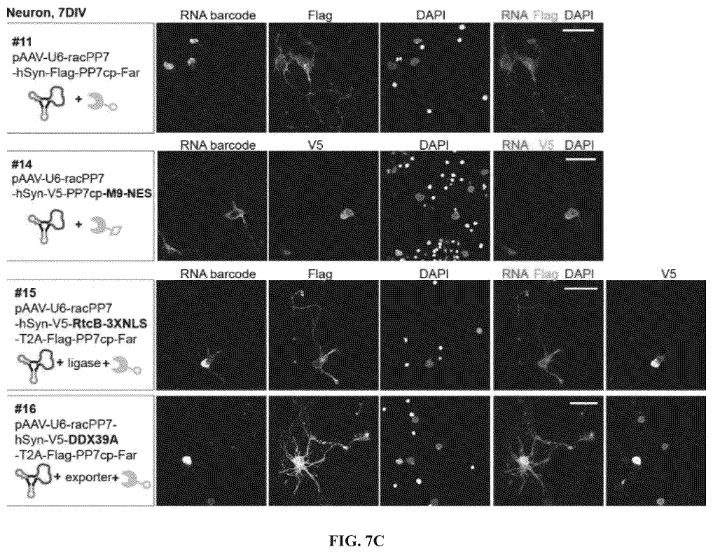

RNA barcode expressing plasmids were introduced into primary rat cortical neurons by electroporation and RNA barcode distribution was assayed via STARmap in 7-14 days (FIG. 7A). Consistent with what was observed in proliferating cells, barcode racPP7 itself remained in the nucleus (FIGS. 7B-7C, row 1). Furthermore, having the barcode in the terminal-helix form or co-expressing RtcB or DDX39A had minimal effects on RNA barcode export (FIG. 7B, row 2; FIG. 7C, rows 3,4). In contrast, hCTE and M9-NES promote RNA barcode export in cultured neurons (FIG. 7B, row 3; FIG. 7C, row 2). Interestingly, rodent cytoplasmic non-coding RNA BC1 but not the primate counterpart BC200 was observed to promote racRNA export in rat cortical neurons (FIG. 7B, rows 4,5), suggesting rodent-specific mechanisms in BC1 localization.

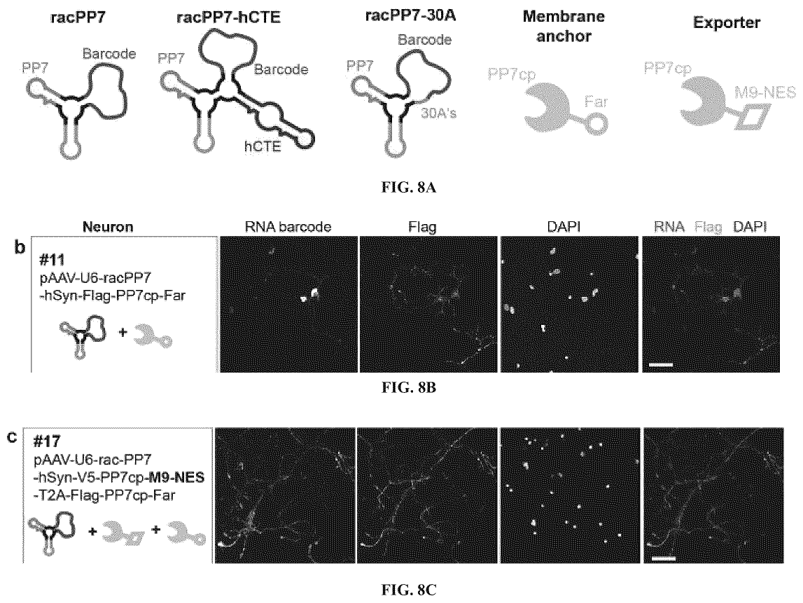

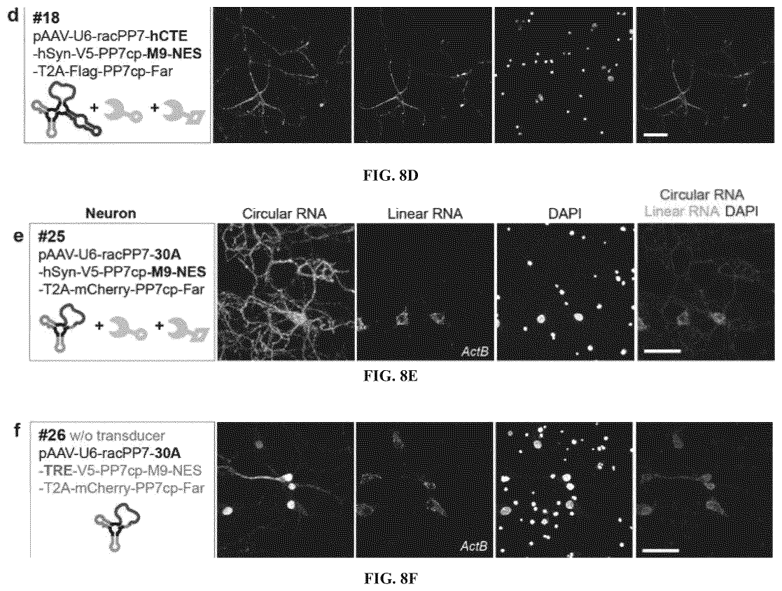

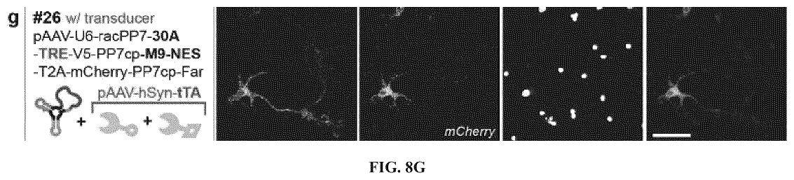

Combining hCTE and M9-NES further facilitated circular RNA barcode export in neurons (FIGS. 8A-21D). To expand the scope of racRNA barcode application, the following derivative vectors were also constructed. (1) racRNA with a 30A stretch which not only exhibits extraordinary copy numbers and cytoplasmic distribution in the STARmap assay (FIGS. 8A and 8E) but also enables co-detection in single-cell RNA-sequencing methods based on oligo(dT). (2) a tTA-dependent system where racRNA barcode export depends on the co-expression of the tTA-regulated exporter M9-NES: nuclear-retaining of racRNA barcodes was vastly diminished when tTA was expressed in the same cell (FIGS. 8F-8G). Note that circular RNA barcode is substantially more abundant than that of linear RNAs such as endogenous rat ActB mRNA or trans-expressed mCherry mRNA (FIGS. 8E-8F), confirming the remarkable stability of RNA barcodes in the circular form.

Besides membrane tethering, a panel of constructs for pre- and post-synaptic targeting and axonal and dendritic targeting were also designed (FIGS. 9A-9E). (1) For pre-synapse, tandem PP7 coat protein (tdPP7cp) was fused with presynaptic marker proteins, VAMP2A and SYP1, whose size fits into an AAV genome (FIG. 9A). They were further combined with the nuclear exporter PP7cp-M9-NES. (2) For excitatory post-synapse, two strategies were utilized: (a) fusing tdMS2cp with excitatory post-synaptic marker protein homer1c; and (b) fusing tdMS2cp with a Fibronectin intrabody (FingR) of excitatory post-synaptic marker protein PSD95 (FIG. 9B). (3) In addition, the second strategy was also implemented for inhibitory post-synapse where λN peptide was fused with FingR of GPHN (FIG. 9C). A negative-feedback transcriptional control was also included in the FingR design to allow for appropriate FingR expression levels to label dendritic spines specifically. (4) Finally, two constructs were designed for pan-dendritic targeting, using the dendritic protein ARC or dendritic RNA BC1 (as discussed above) (FIG. 9D). racRNA barcode was decently exported for homer1c (FIG. 9E) and ARC without M9-NES, likely due to the intrinsic nuclear-cytoplasmic shuttling properties of the two proteins. Representative RNA barcode distributions in neurons from those constructs were shown in FIG. 9E.

### Example 5: Demonstration In Vivo in the Adult Mouse Brain

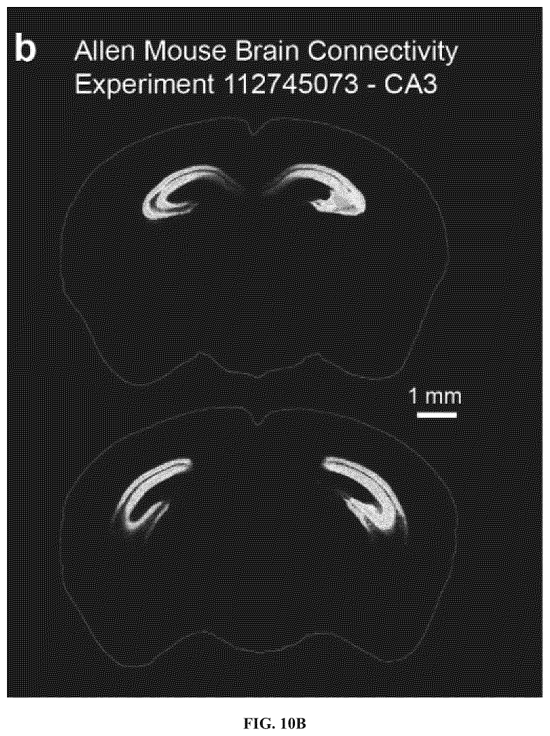

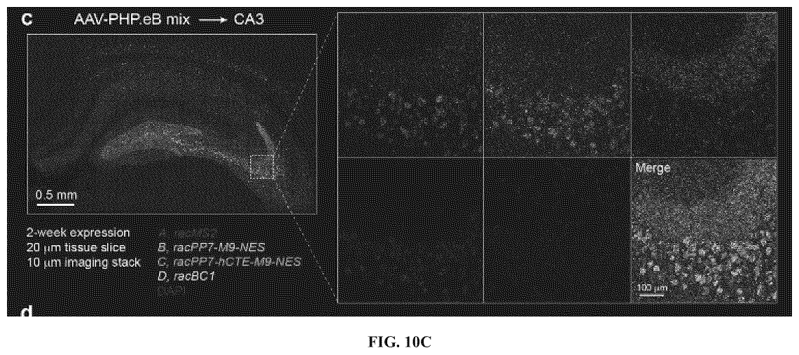

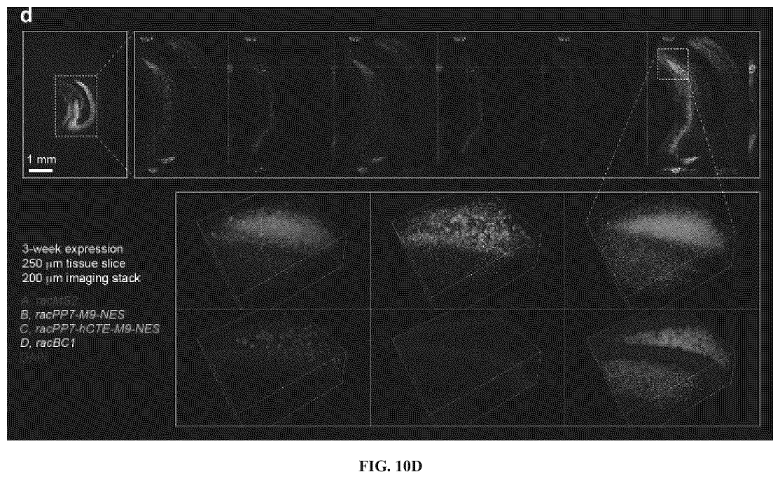

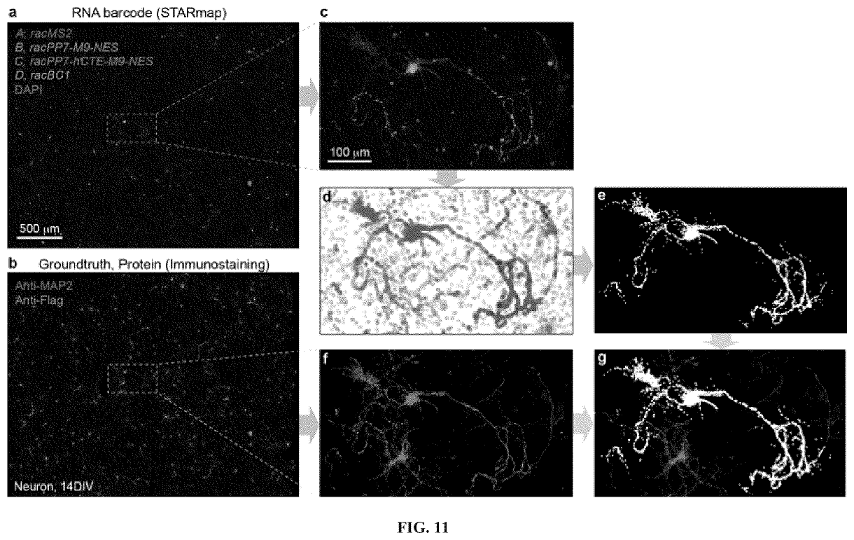

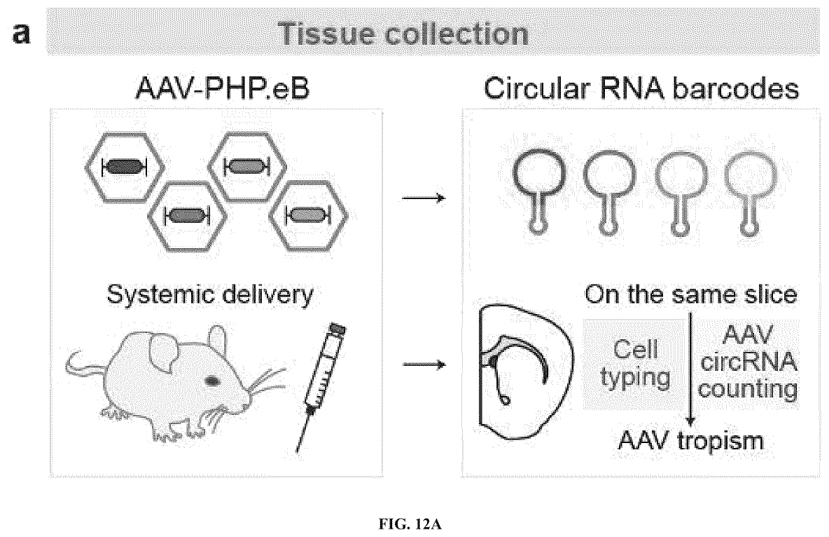

Next, four designs of RNA export plasmids were tested in the same sample in vivo, including the non-export design (racMS2), a cis-element BC1 (racBC1), a trans-element M9-NES (racPP7-M9-NES), and the combined design of the cis-element hCTE and the trans-element M9-NES (racPP7-hCTE-M9-NES). To do so, each plasmid was labeled with a unique barcode and packaged into recombinant adeno-associated virus (rAAV, serotype AAV-PHP.eB) (FIG. 10A). Finally, the AAV mix was injected in the CA3 region of the adult mouse brain and the RNA barcode distribution was assayed in thin (20 μm) and thick (250 μm) mouse brain slices after 2-3 weeks of expression. Injections were made at the CA3 region due to the synchronized projection of CA3 granule neurons towards CA1 (FIG. 10B) so that exported and membrane-anchored RNA barcodes would show tissue-level patterns.

The export strategies held in vivo as well (FIGS. 10C-10D). In contrast to the non-export design (racMS2) that mostly remained in the nucleus and filled the space of DAPI staining, racBC1 showed distributions in both the nucleus and dendrites, suggestive of dendritic localization of BC1 RNA in rodent neurons. More promisingly, racPP7-M9-NES was distributed in both nucleus and neurites, and racPP7-hCTE-M9-NES was mostly in neurites. To summarize, effective constructs were provided to label subcellular compartments (nucleus v.s. cytoplasm; soma v.s. neurites; dendrites v.s. neurites) and cell morphology.

### Example 6: Barcoding Cells with racRNAs for Morphological Tracing and Lineage Tracing

Circular RNA barcodes were utilized to achieve single-cell resolved morphological tracing. Compared to protein-based cell morphology mapping methods (such as Brainbow) which are limited by the number of spectrum-resolvable fluorescent proteins, RNA-based barcoding allows for substantially higher multiplexity via its combinatorial sequences. Meanwhile, the abundance and stability of the racRNA demonstrated above make it an ideal barcode carrier. RNA-barcode-assisted morphological tracing would be beneficial for accurate cell segmentation in imaging-based spatial transcriptomics methods and integrative analysis of single-cell transcriptome and morphology.

As a demonstration, primary rat cortical neuronal cultures were used. Four of the RNA export and/or membrane-tethering plasmid constructs were electroporated into four neuronal populations, respectively, and the neurons were co-cultured for 14 days. STARmap was performed to detect racRNA barcode distribution in situ, followed by immunostaining of the Flag-tagged membrane anchor protein to acquire ground-truth cell morphology of the same sample (images A-C and F of FIG. 11). Next, ClusterMap (He Y, et al., “ClusterMap for multi-scale clustering analysis of spatial gene expression,” Nat Commun 12: 5909 (2021), the disclosure of which is incorporated herein by reference in its entirety for all purposes), a computational pipeline that segments cells based on spot density and identity, was applied to racRNA barcode amplicon spots identified from the raw image (image D of FIG. 11), resulting in a cell determined by racRNA barcodes (image E of FIG. 11). Importantly, different from endogenous mRNA amplicons that are concentrated in the cell body, the cell identified by racRNA barcodes exhibits extended morphological features such as dendrites and long axons (image E of FIG. 11), which aligned well with ground-truth protein staining (image G of FIG. 11).

In addition to the membrane-tethered version of racRNA barcodes, nuclear-localized racRNA barcodes can be well compatible with single-nuclear sequencing applications and imaging applications such as lineage tracing (see, e.g., Van Vliet K M, et al. “The role of the adeno-associated virus capsid in gene transfer,” Methods Mol Biol 437: 51-91 (2008), the disclosure of which is incorporated herein by reference in its entirety for all purposes).

### Example 7: Connectome Mapping in Animal Models

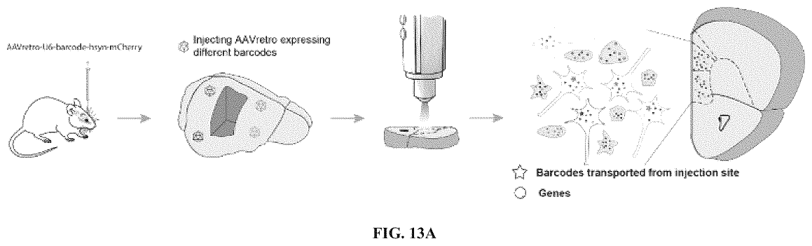

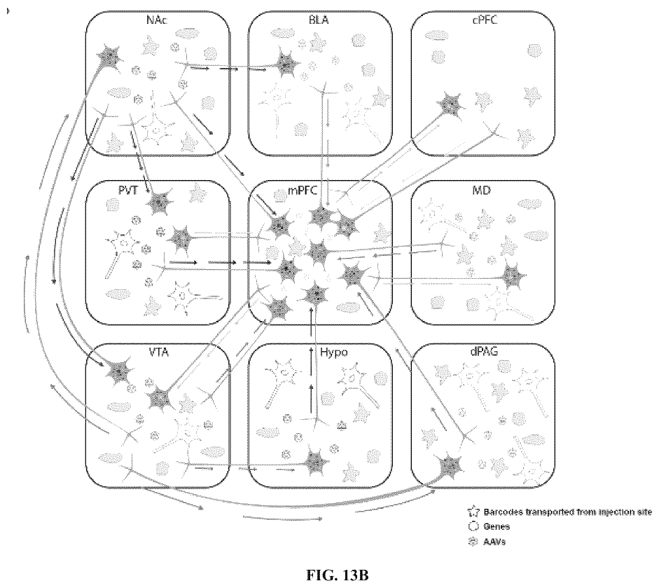

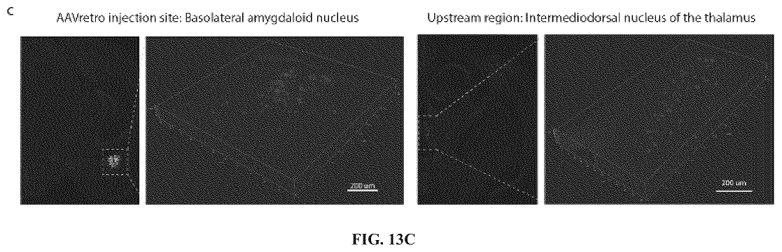

Projecting targets of individual neurons are critical features of the brain connectome. Current projection mapping strategies include anterograde tracing by expressing fluorescent proteins on axons and retrograde tracing by injecting retrograde tracer (e.g., CTB) or virus (e.g., pseudorabies) into the downstream regions. However, all those strategies are limited by the throughput. The projecting pattern of different neuronal types needs to be mapped one by one in different mice. Furthermore, retrograde tracers can only be injected into, at most, 3 regions because of the color channel limitations. By applying AAVretro (Tervo, et al., Neuron 2016; 92: 372-382) to deliver barcoded racRNA from injection regions to their upstream regions (FIG. 13A), single-neuron resolution and high throughput in mapping projection targets were achieved within the brain. For example, nine interconnected brain regions were selected and nine different AAVretro racRNA barcodes were injected into these regions individually (FIG. 13B). The barcodes in each region can be retrogradely transported to upstream regions to label the projecting neurons targeting barcode-injected regions. Single-neuron projection targets could be delineated by decoding the barcodes which are orthogonal to the locally injected barcode and represent the targeted downstream brain regions. As shown in FIG. 13C, AAVretro racRNA were injected containing a specific barcode into the basolateral amygdaloid nucleus (BL). This barcode was detected in the upstream region, inter-mediodorsal nucleus of the thalamus (IMD), which indicates that those labeled neurons in IMD have projections to BL. Theoretically, unlimited projection targets can be mapped of multiple brain regions simultaneously within one mouse, which would be super beneficial for understanding the structure of the brain connectome.

### Example 8: Spatial Atlas of the Mouse Central Nervous System at Molecular Resolution

Deciphering spatial arrangements of molecular cell types at single-cell resolution in the nervous system is fundamental for understanding the molecular architecture of its anatomy, function, and disorders. While single-cell RNA-sequencing (scRNA-seq) has revealed the complexity and diversity of cell-type composition in the mouse brain, it provides little to no spatial information. Emerging spatial transcriptomic methods have shed light on the molecular organization of mouse brains. However, existing datasets either have limited spatial resolution (100 μm)—hindering bonafide single-cell analysis—or are restricted to particular brain subregions. Therefore, a comprehensive, single-cell resolved spatial atlas across the entire CNS is highly desirable to fully unveil molecular cell types and tissue architectures.

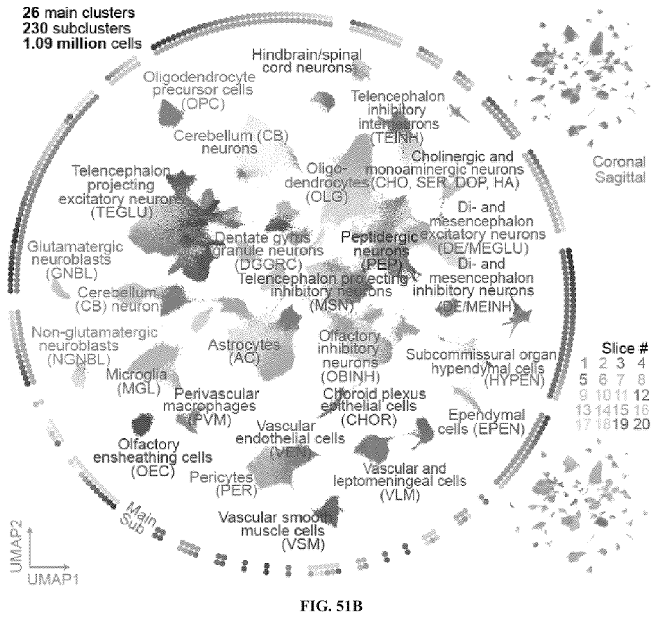

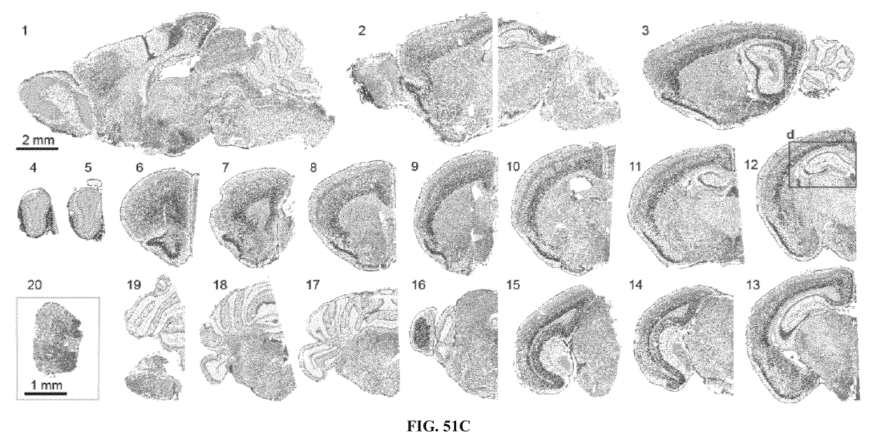

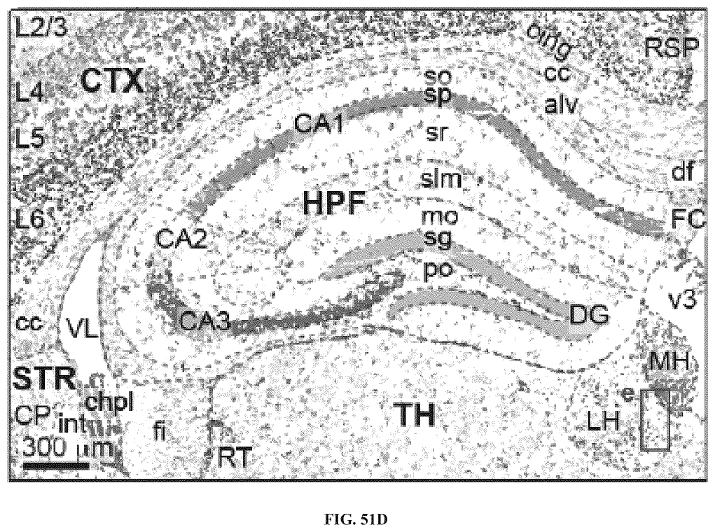

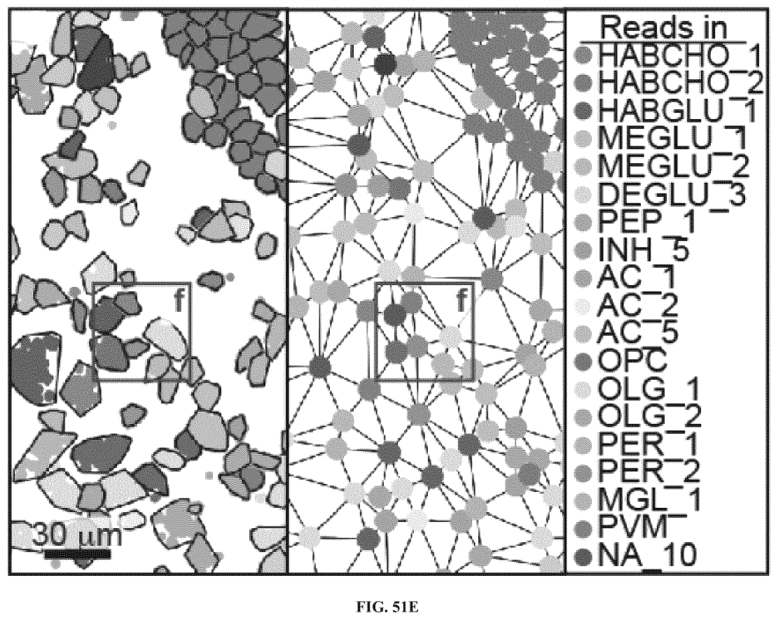

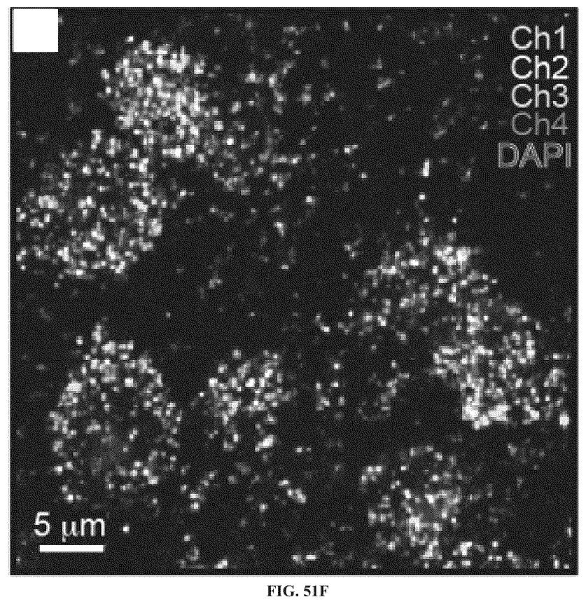

Accordingly, experiments were undertaken to use STARmap PLUS to detect 1,022 endogenous genes in 20 CNS tissue slices in situ at a voxel size of 194×194×345 nm³ followed by ClusterMap cell segmentation. By integrating with a published scRNA-seq atlas, molecular cell type maps were generated based on single-cell gene expression and molecular tissue region maps were generated based on spatial niche gene expression, which allowed a joint definition of brain-wide molecular spatial cell nomenclatures. Furthermore, transcriptome-wide, spatially resolved single-cell expression profiles were imputed. These experiments facilitated the development of a comprehensive molecular spatial atlas for mouse CNS, comprising over one million cells with their transcriptome-wide gene expression profiles, spatial coordinates, molecular cell types, molecular tissue regions, and joint cell type nomenclature (FIG. 51A). As an application of the mouse molecular CNS spatial atlas, a highly efficient RNA barcoding system was developed and combined with STARmap PLUS to chart the tissue and cell-type transduction landscapes of PHP.eB, an engineered recombinant adeno-associated virus (rAAV) strain that can penetrate the blood-brain barrier through systemic administration. Altogether, experimental and computational frameworks were developed for establishing a molecular spatial atlas across various scales, from individual RNA molecules to single cells to tissue regions.

### Example 8.1: Spatial Maps of CNS Molecular Cell Types

STARmap PLUS is an image-based in situ RNA sequencing method (Wang, X. et al. Science 361, eaat 5691 (2018); Zeng, H. et al. Nat. Neurosci. (2023) doi:10.1038/s41593-022-01251-x) that utilizes paired primer and padlock probes (SNAIL probes) to convert target RNA molecules into DNA amplicons with gene-unique codes, which enables highly multiplexed RNA detection in tissue hydrogel by multiple rounds of sequencing by ligation with error rejection (SEDAL seq) (FIG. 51A).

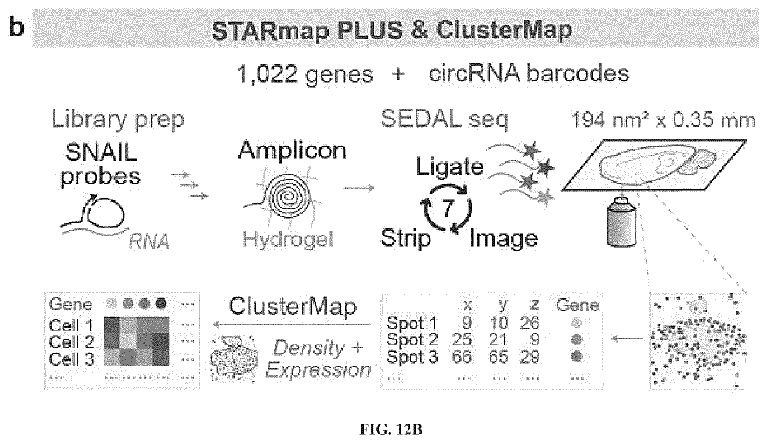

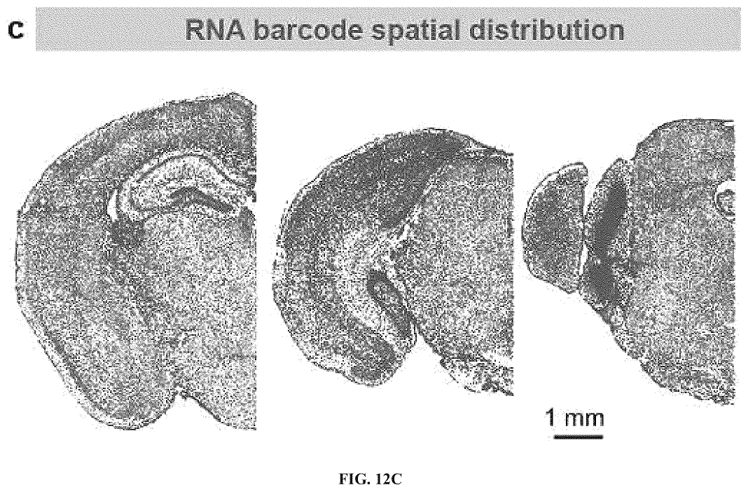

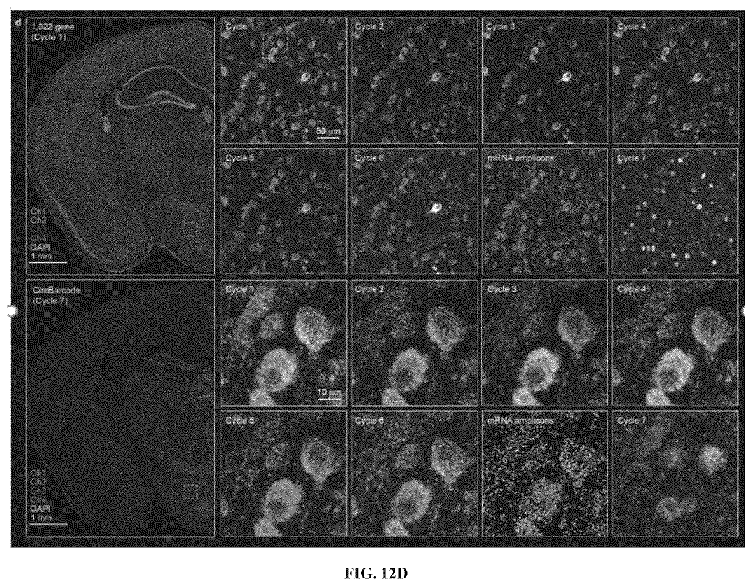

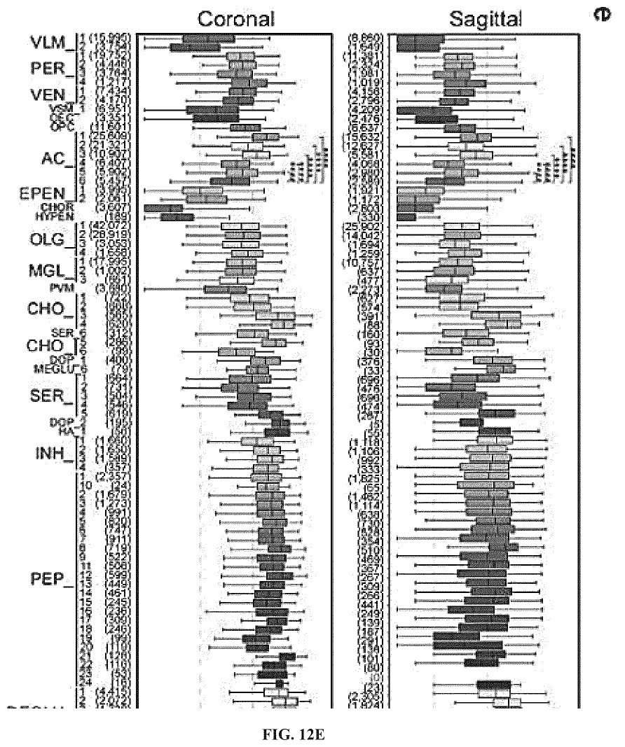

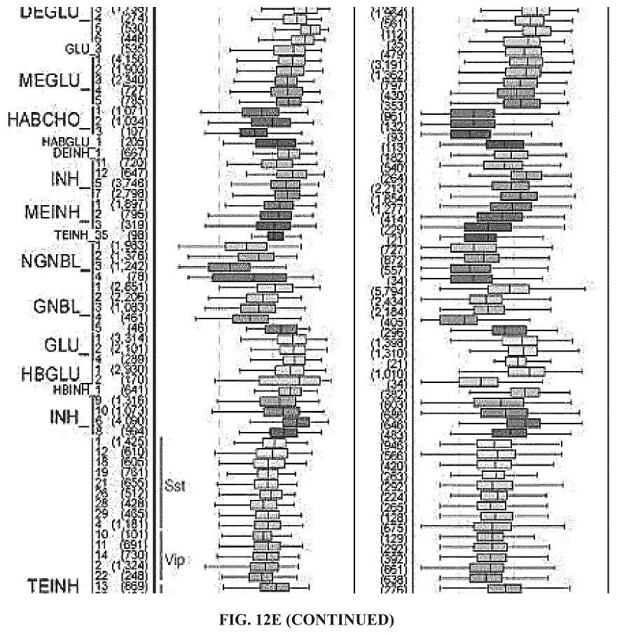

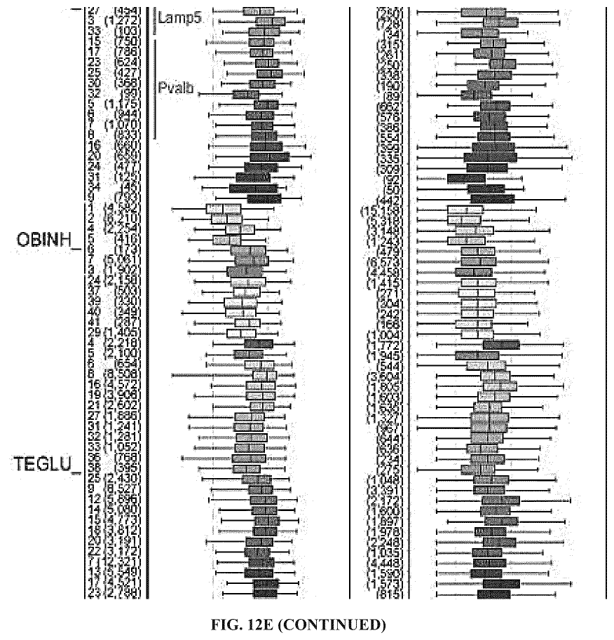

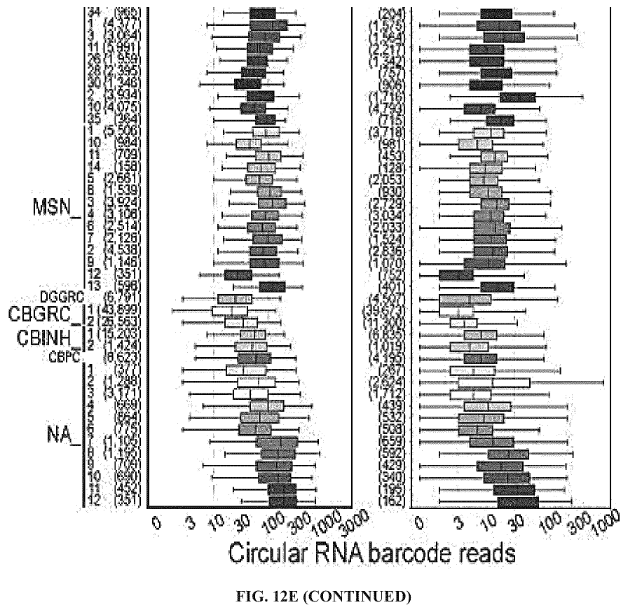

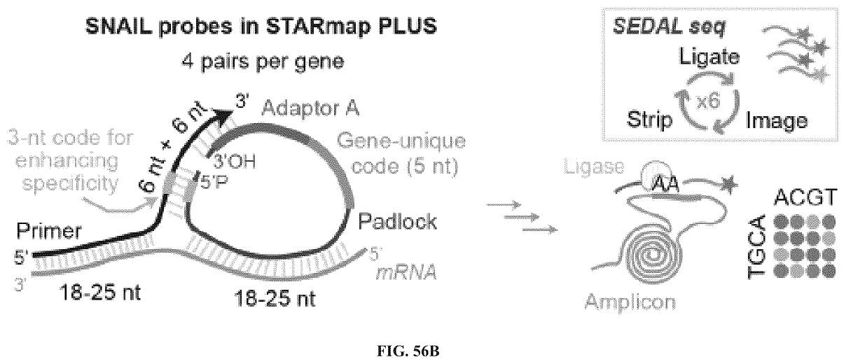

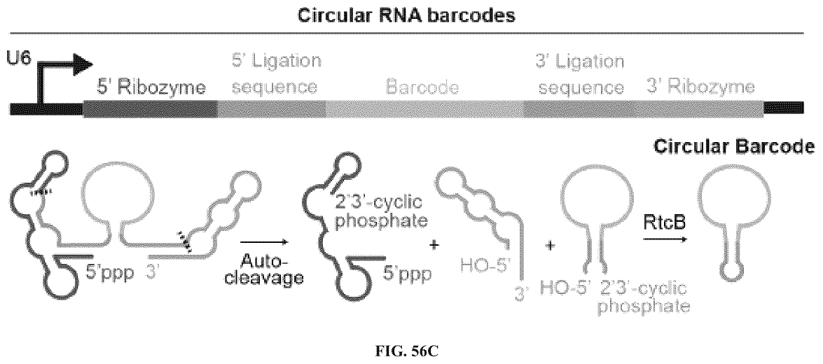

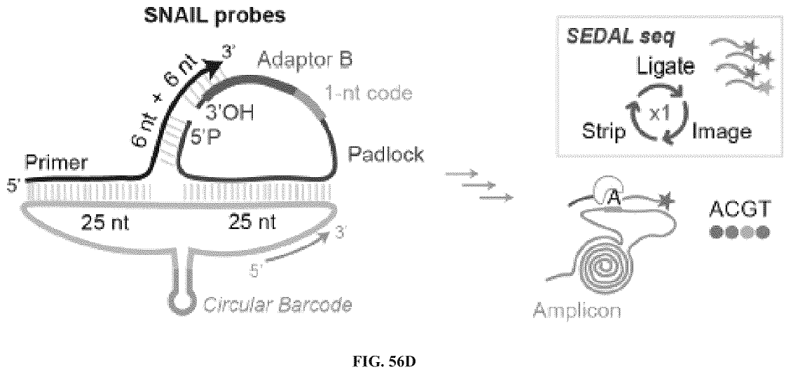

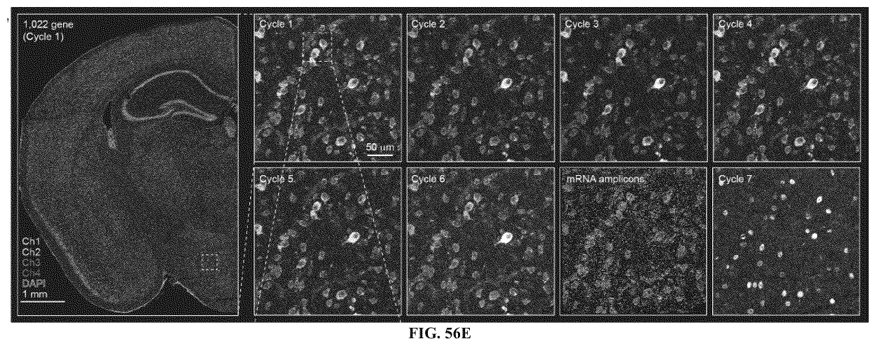

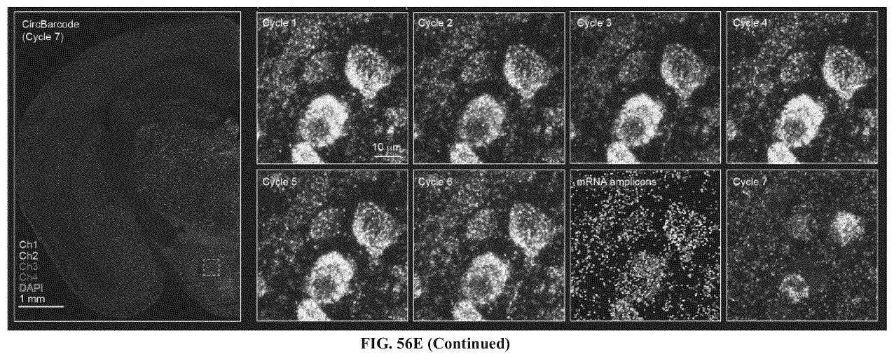

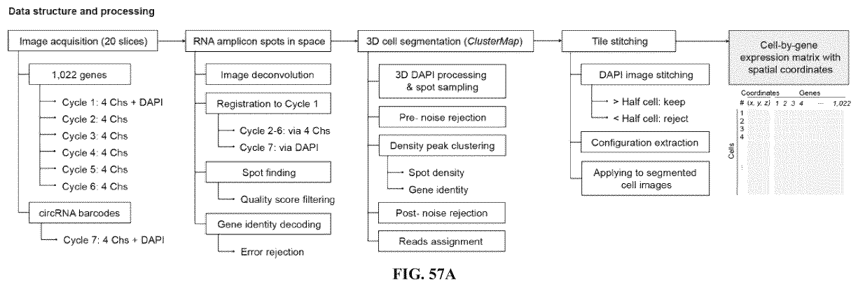

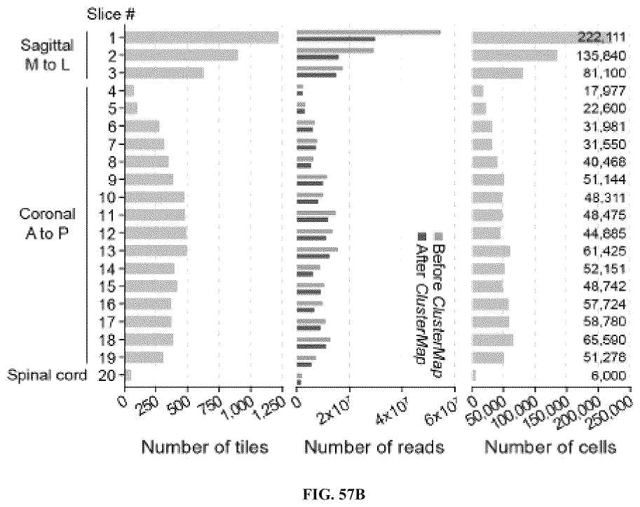

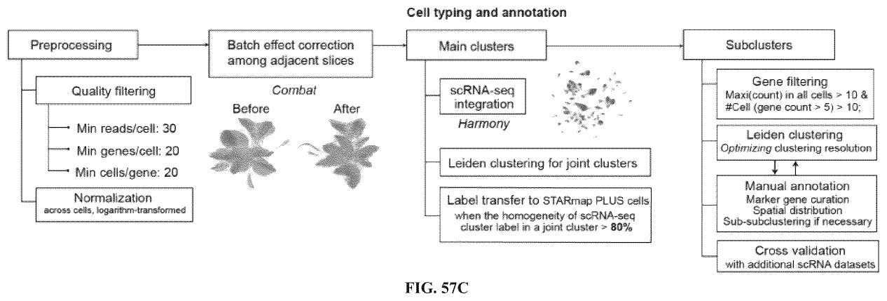

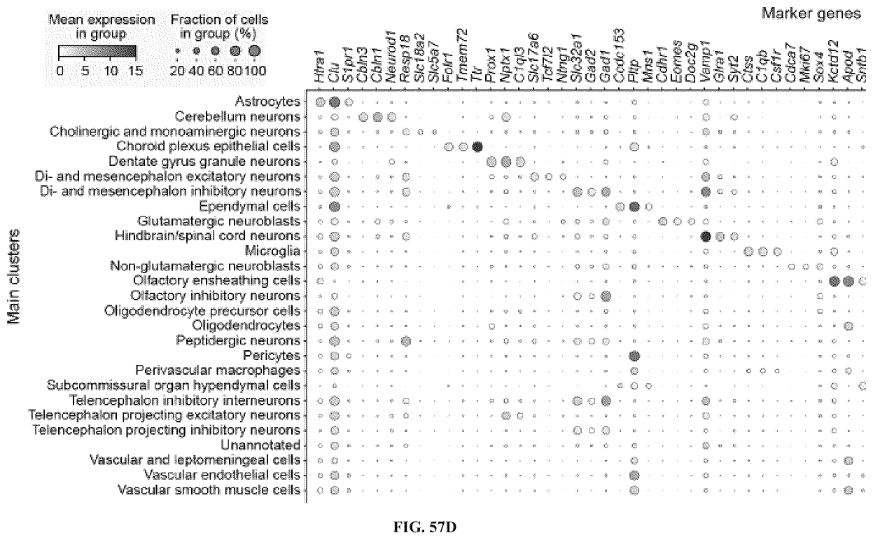

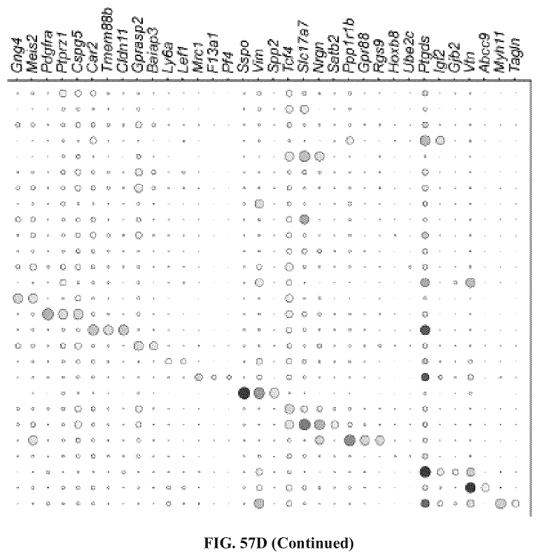

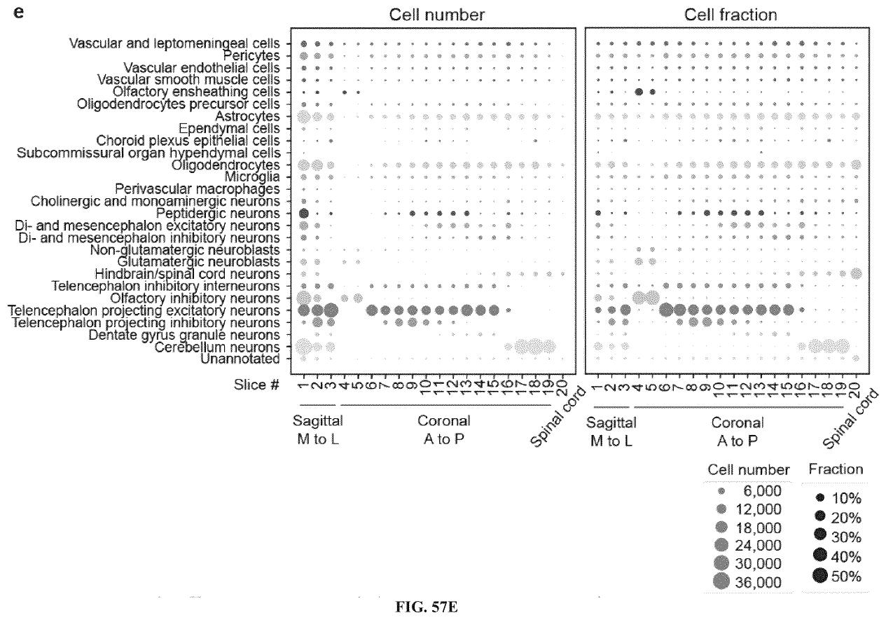

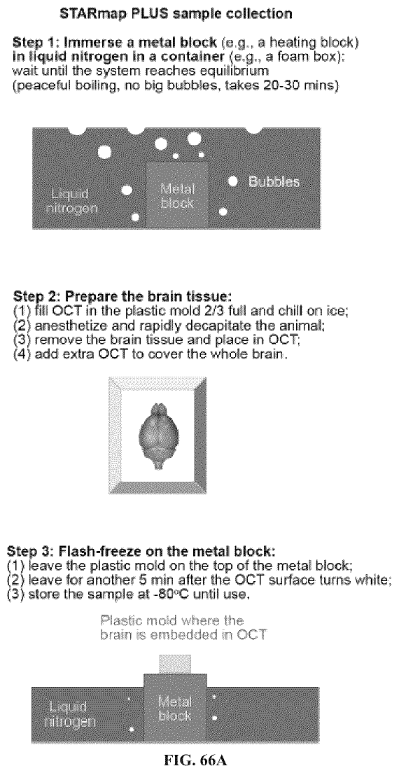

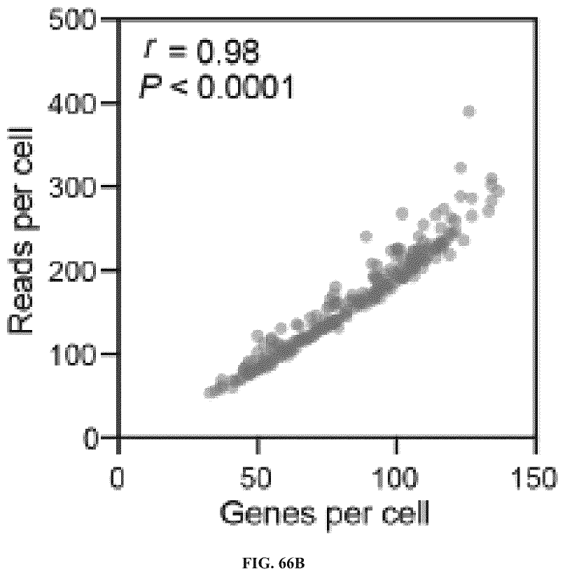

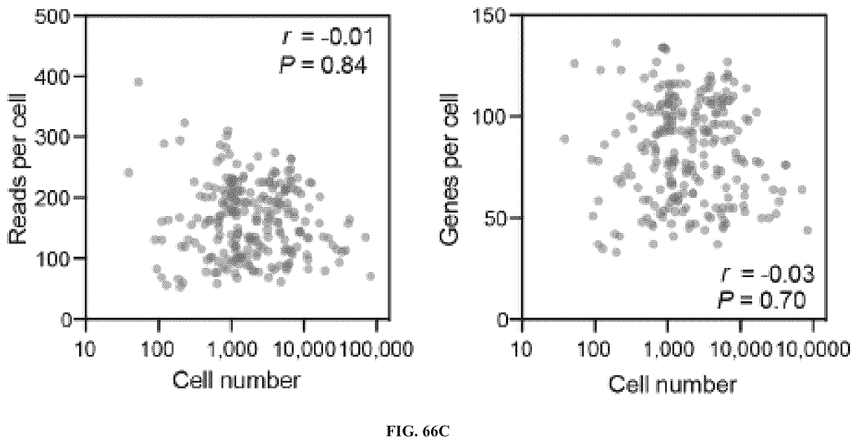

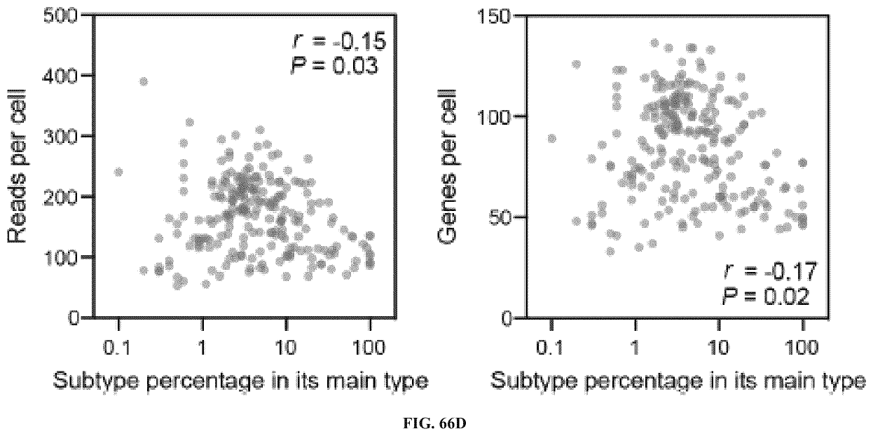

To achieve CNS-wide molecular cell typing, the following list of 1,022 genes (FIG. 56A) by compiling reported cell-type marker genes from adult mouse CNS scRNA-seq datasets with minimal post-dissection cell-type selection: A2m, Abcc9, Abi3 bp, Acbd7, Acta2, Ada, Adamts15, Adarb2, Adcy1, Adcyap1, Adcyap1r1, Adgrg2, Adgrg6, Adm, Adora1, Adora2a, Adora2b, Adora3, Adra1b, Adrb1, Adrb2, Adrb3, Afp, Agrp, Agt, Agtr2, Ajap1, Alcam, Aldh3b2, Angpt2, Angpt4, Ankrd34b, Anln, Anpep, Anxa1, Anxa11, Apln, Aplnr, Apoc1, Apod, Apold1, Aqp1, Aqp4, Arap2, Areg, Arg1, Arhgap25, Arhgap36, Arhgap6, Arsj, Asb4, Asic3, Asic4, Ass1, Atf3, Atp2a3, Atp2b4, Avp, B3gat2, Baiap2l1, Baiap3, Barhl1, Bcl11b, Bc/6, Bdkrb1, Bdkrb2, Bdnf, Bhlhe22, Birc2, Birc5, Bmp3, Bmp4, Brcal, Brs3, C1qb, C1ql1, C1ql2, C1ql3, C1qtnf7, C4b, Cabp7, Cacna2d1, Cacna2d2, Cacng4, Cadm1, Cadm2, Calb1, Calb2, Calca, Calcb, Calcr, Calcrl, Camk2d, Car10, Car2, Car3, Car4, Car8, Card10, Cartpt, Casp4, Casp8, Casr, Cb/n1, Cb/n2, Cb/n3, Cb/n4, Cbr2, Cbs, Ccdcl53, Cck, Cckar, Cckbr, Cc/24, Cc/3, Ccl4, Ccl7, Ccna1, Ccnd1, Ccne1, Ccp110, Ccr6, Ccrl2, Ccsap, Cd74, Cd9, Cd93, Cdc20, Cdca7, Cdh13, Cdh7, Cdhrl, Cdk1, Cdkl4, Cdkn1c, Cdkn2b, Ceacam10, Cemip, Cenpf, Cfap126, Cfap58, Cfh, Chat, Chodl, Chrm1, Chrm2, Chrm3, Chrm4, Chrm5, Chrna2, Chrna3, Chrna6, Chrnb3, Cited1, Cks2, Clca3a1, Cldn10, Cldn11, Cldn19, Cldn5, Clec2l, Clic5, Clic6, Clu, Cnksr3, Cnn1, Cnpy1, Cnr1, Cnr2, Cntnap3, Coch, Col11a1, Col12a1, Col15a1, Col18a1, Col19a1, Col20a1, Col24a1, Col25a1, Col3a1, Col5a1, Col6a1, Col9a2, Coro6, Cort, Cox4i2, Cox6a2, Cox8b, Cpa6, Cpb1, Cplx3, Cpne4, Cpne5, Cpne6, Cpxm2, Crabp1, Crct1, Creb3l1, Crh, Crhbp, Crhr1, Crhr2, Crim1, Crisp1, Crispid2, Crym, Csflr, Cspg5, Csrp2, Cst3, Cips, Ctsc, Ctss, Ctxn3, Cux2, Cxcl1, Cxcl14, Cxcl2, Cyp26b1, Cyp2s1, Cyth3, Dad1, Dapl1, Dbh, Dbpht2, Dclk3, Dcn, Ddit4l, Degs2, Deptor, Dgkk, Dhh, Dkk1, Dkk3, Dkkl1, Dlk1, Dlx1, Dlx2, Dlx5, Dmbx1, Dmkn, Doc2g, Dock5, Dpt, Dpy1911, Drdl, Drd2, Drd3, Drd4, Drd5, Dynlrb2, Ebf1, Ebf2, Ebf3, Ecel1, Ecscr, Edn3, Ejhd1, Ejhd2, Efna5, Egln3, Elfn1, Emid1, Emx2, En1, Enpp6, Eomes, Epha7, Epyc, Espn, Esrrg, Etv1, F13a1, Fabp7, Fam107a, Fam169b, Fam180a, Fam181b, Fam183b, Fam184a, Fam214a, Fam216b, Fam92b, Fat2, Fbin2, Fbin5, Fbp2, Fcmr, Fermt1, Fev, Fezf1, Fezf2, Fgf10, Fgfr3, Fibcd1, Fign, Fjx1, Flt1, Fn1, Folr1, Fos, Foxp2, Frzb, Fshr, Fst, Gabbr1, Gabbr2, Gabra5, Gabra6, Gabrq, Gabrr2, Gad1, Gad2, Gadd45a, Gal, Galnt14, Galnt16, Galr1, Galr2, Galr3, Gast, Gata3, Gbx1, Gbx2, Gcgr, Gch1, Gchfr, Gdf10, Gfap, Gfra1, Gfra2, Gfra3, Ghrh, Ghrhr, Ghsr, Gipr, Gja1, Gjb1, Gjb2, Gkn3, Gldc, Glra1, Gm5741, Gna14, Gnb3, Gng4, Gng8, Gnrhl, Gnrhr, Gpc3, Gpr101, Gpr119, Gpr139, Gpr17, Gpr34, Gpr50, Gpr83, Gpr88, Gprasp2, Gpsm1, Gpsm3, Gpx2, Gpx3, Grik1, Grik3, Grin2c, Grin1, Grm2, Grm3, Grm4, Grm5, Grm6, Grm7, Grm8, Grp, Grpr, H2-ab1, Hand1, Hap1, Hapln1, Hapln2, Hcrt, Hcrtr1, Hcrtr2, Hdc, Hdhd3, Hhip, Higd1b, Hopx, Hoxa10, Hoxa5, Hoxa7, Hoxa9, Hoxb3, Hoxb5, Hoxb6, Hoxb7, Hoxb8, Hoxb9, Hoxc10, Hoxc4, Hoxc5, Hoxc8, Hoxc9, Hpcal1, Hpcal4, Hrh1, Hrh2, Hrh3, Hrh4, Hs3st2, Hs3st4, Hs6st2, Hspa1a, Hspb7, Htr1a, Htr1b, Htr1d, Htr1f, Htr2a, Htr2b, Htr2c, Htr3a, Htr3b, Htr5a, Htr5b, Htra1, Ibsp, Id2, Id4, Ido1, Ifitm1, Igf2, Igfbp2, Iglbp4, Iglbp6, Igfbp11, Igsf1, Igsf8, Illr1, I1rapl2, I123a, I131ra, Il33, Inhba, Inmt, Inpp5j, Insrr, Irs4, Irx2, Irx4, Irx6, Isl1, Isi2, Islr, Itih3, Itk, Itpr2, Iyd, Junb, Kcnab1, Kcnc2, Kcnc3, Kcnd3, Kcng1, Kcng4, Kcnh8, Kcnip1, Kcnj8, Kcnk3, Kcnmb1, Kcnmb2, Kcns1, Kctd12, Kif5b, Kiss1r, Kit, Kitl, Kl, Klhl1, Klh14, Klk6, Krt12, Krt15, Krt7, Krt19, Krt27, Krt73, Lamp5, Lancl3, Lbp, Lbx1, Lcn2, Lef1, Lefty1, Lgi2, Lhx1, Lhx2, Lhx6, Lhx8, Lhx9, Lims2, Lingo4, Lmcd1, Lino1, Lmo3, Lmx1a, Lpar3, Lpl, Lrg1, Lrpprc, Lrrc55, Lrrtm2, Lsamp, Ltk, Lum, Ly6a, Ly6c1, Ly6d, Ly6g6e, Lypd1, Lypd2, Lypd6, Lypd6b, Mab21i2, Mal, Man1a, Maob, Map3k7cl, Matn2, Mbp, Mc1r, Mchr1, Mdga1, Megf11, Meis2, Meox1, Mfap4, Mfge8, Mfsd2a, Mgarp, Mgp, Mgst1, Mia, Mki67, Mlc1, Mlf1, Mmp2, Mns1, Mog, Moxd1, Mpz, Mrap2, Mrc1, Mreg, Mrgpra3, Mrgprd, Ms4a15, Ms4a7, Mtnr1a, Mtnr1b, Mustn1, Myc, Myh11, Myh8, Myl1, Myl4, Myoc, Nccrp1, Ncmap, Ndnf, Ndrg2, Ndst4, Ndufa4i2, Necab1, Nefh, Nefm, Nell1, Neu4, Neurod1, Neurod2, Neurod6, Nfatc2, Nfib, Ngb, Ngfr, Nhlh2, Ninj2, Nkx2-1, Nkx2-9, Nmb, Nmbr, Nms, Nmu, Nmur1, Nmur2, Nog, Nos1, Notum, Npas1, Npbwr1, Npff, Npffr1, Npffr2, Npnt, Nppa, Nppb, Nppc, Npsr1, Nptx1, Nptx2, Npw, Npy, Npy1r, Npy2r, Npy4r, Npy5r, Nr2f2, Nr3c2, Nr4a2, Nr4a3, Nrep, Nrgn, Nrip3, Nrl, Nrp2, Nrtn, Ntf3, Ntng1, Ntrk1, Nts, Ntsr1, Ntsr2, Nwd2, Nxph1, Nxph2, Nxph3, Nxph4, Nyap2, Olfm2, Olfml2a, Olfr558, Omp, Onecut2, Opalin, Oprd1, Oprk1, Opri1, Oprm1, Oscp1, Osr1, Otoa, Otof, Otp, Otx1, Otx2, Oxtr, P2rx2, P2ry12, Pak4, Palm3, Pappa, Pappa2, Paqr5, Parm1, Parp14, Pax2, Pax5, Pax6, Pax7, Pax8, Pbk, Pbx3, Pcdh11x, Pcdh20, Pcp2, Pcp4, Pcsk5, Pdcd4, Pde11a, Pde1a, Pde1c, Pde6g, Pdgfa, Pdgfra, Pdlim1, Pdyn, Pdzk1ip1, Peg10, Penk, Pf4, Pgam2, Pglyrp1, Pgr, Pgr15l, Phlda1, Phox2a, Phox2b, Pi16, Piezo2, Pik3r3, Pitx2, Pkd112, Pkd2l1, Pkib, Pla2g5, Plchl, Plcxd2, Pin3, Pltp, Pmch, Pnmt, Pnoc, Pomc, Postn, Pou3f1, Pou4f1, Pou4j2, Pou4f3, Pou6j2, Ppm1j, Ppplr14a, Ppplr17, Ppp1r1b, Ppp1r3g, Ppp2r2b, Prc1, Prdm12, Prkcd, Prkcg, Prlh, Prlhr, Prlr, Procr, Prok2, Prokr1, Prokr2, Prox1, Prph, Prr51, Prrxl1, Prss12, Prss23, Prss35, Prss56, Prx, Ptgds, Ptgfr, Ptgir, Pthir, Pth2r, Pthlh, Ptpn3, Ptprk, Ptprz1, Pvalb, Pyy, Rab37, Rab3b, Ramp3, Rarres1, Rasd1, Rasl10a, Rasl11a, Rbp4, Rd3l, Rell1, Reln, Rerg, Resp18, Ret, Rgs12, Rgs14, Rgs16, Rgs4, Rgs5, Rgs8, Rgs9, Rhcg, Rims4, Rin1, Rln3, Rnfl52, Rora, Rorb, Rpp25, Rprm, Rps24, Rras2, Rrm2, Rspo1, Rspo3, Runx1, Rxfp1, Rxfp2, Rxfp3, Rxfp4, Rxrg, S100a4, S1pr1, Sag, Sal13, Samsn1, Sapcd2, Satb1, Satb2, Scgb3al, Scgn, Scn10a, Scn4b, Scn5a, Scn7a, Scnn1a, Sctr, Scube1, Selpig, Sema3a, Sema3c, Sema3e, Sema3f, Sema3g, Sema4d, Sema5a, Serpinb1a, Serpinb1b, Serpinf1, Sez6, Sfrp2, Shisa8, Shox2, Siglech, Sim1, Six3, Six6, Skor1, Sla, Sla2, Slc13a3, Slc17a6, Slc17a7, Slc17a8, Slc18a2, Slc18a3, Slc1a3, Slc1a6, Slc22a4, Slc24a2, Slc26a3, Slc30a3, Slc32a1, Slc34a2, Slc36a2, Slc47a1, S1c5a7, Slc6a11, Slc6a13, Slc6a2, Slc6a3, Slc6a4, Slc6a5, Slc7a10, Slco3al, Sln, Smim17, Smoc1, Sncg, Sntb1, Snx33, Socs3, Sores1, Sost, Sostdc1, Sox11, Sox14, Sox4, Sp9, Sparc, Spdef, Sphkap, Spink8, Spon1, Spon2, Spp1, Spp2, Sspo, Sst, Sstr2, St18, St8sia4, St8sia6, Stac2, Stard8, Steap2, Stk32b, Stmn2, Sulf1, Sulf2, Sumo2, Sv2c, Synpo2, Synpr, Syt15, Syt2, Syt6, Tac1, Tac2, Tacr1, Tacr2, Tacr3, Tacstd2, Tagln, Tal1, Tax1bp3, Tbr1, Tbx18, Tbx20, Tbxa2r, Tcap, Tcerg1l, Tcf4, Tcf712, Teddm3, Tek, Tekt5, Tfap2b, Tfap2c, Tfap2d, Tgfb2, Th, Thbd, Thrsp, Tiam1, Tiam2, Timp4, Tlx3, Tm4sf4, Tmc3, Tmem114, Tmem119, Tmem132c, Tmem141, Tmem163, Tmem212, Tmem215, Tmem233, Tmem255a, Tmem255b, Tmem26, Tmem45b, Tmem54, Tmem72, Tmem88b, Tmsb4x, Tnf Tnfrsf13c, Tnnc1, Tnni3, Tnnt1, Tnnt3, Tnr, Top2a, Tox, Tpbg, Tpd5211, Tph2, Traf3ip3, Trappc3l, Trdn, Trem2, Trf, Trh, Trhr, Trim54, Trim66, Trp73, Trps1, Trpv1, Tshz2, Tspan8, Ttr, Ttyh1, Tuba1c, Tubgcp2, Tyrp1, Ube2c, Ucn, Ucn2, Ucn3, Ugt8a, Unc5b, Ung, Urah, Uts2b, Vamp1, Vcan, Vegfa, Vgl13, Vim, Vip, Vipr1, Vipr2, Vsig8, Vtn, Vwc2, Vwc2l, Vwf Wfdc12, Wfdc18, Wfdc2, Wfs1, Whrn, Wif1, Wnt2, Wnt4, Yjefn3, Zbtb20, Zjhx4, Zfp239, Zic1, Zmym1, Sstr1, and Oxt. A five-nucleotide code on the SNAIL probes encoding gene identity were read out by six rounds of SEDAL seq (FIG. 56B). To allow orthogonal detection of AAV transcripts, highly expressed circular RNA barcodes were designed without homology to mouse transcriptome (FIG. 56B) to be detected by another round of SEDAL seq (FIG. 56D). STARmap PLUS datasets of 20 ten-pm-thick CNS tissue slices were collected from three mice, including sixteen coronal brain slices, three sagittal brain slices, and one transverse slice from spinal cord lumbar segments (FIG. 66A; representative raw fluorescent images in FIGS. 12D and 56E). With an optimized ClusterMap (He, Y. et al. Nat. Commun. 12, 5909 (2021)) data processing workflow, a cell-by-gene expression matrix was generated with RNA and cell spatial coordinates (FIG. 57A). In total, the datasets include 256 million RNA reads and 1.1 million cells (FIG. 57B).

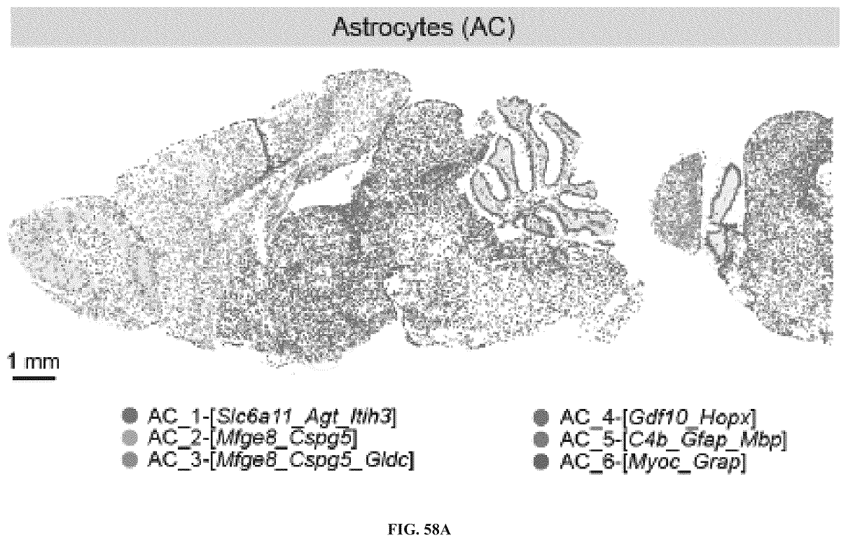

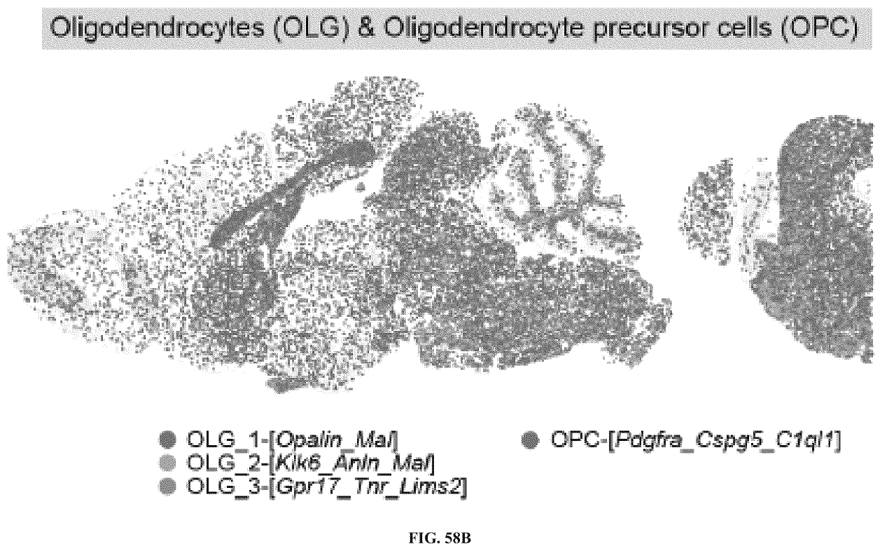

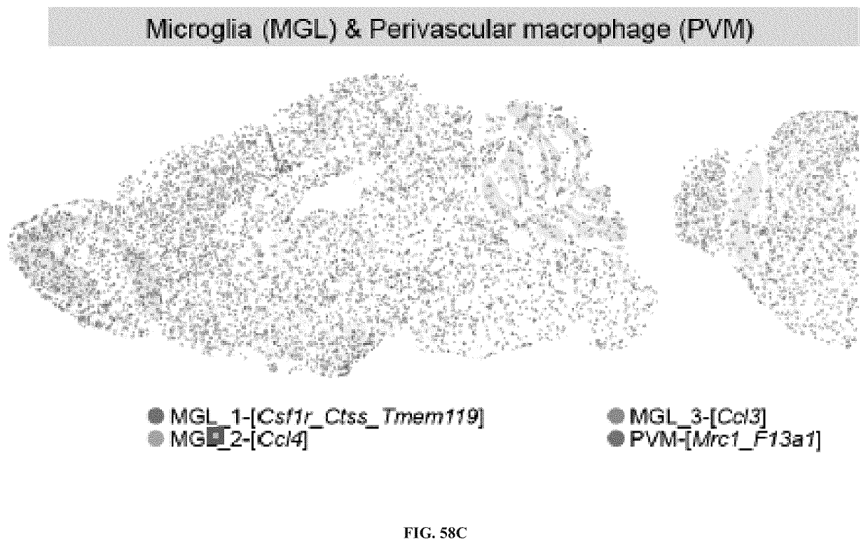

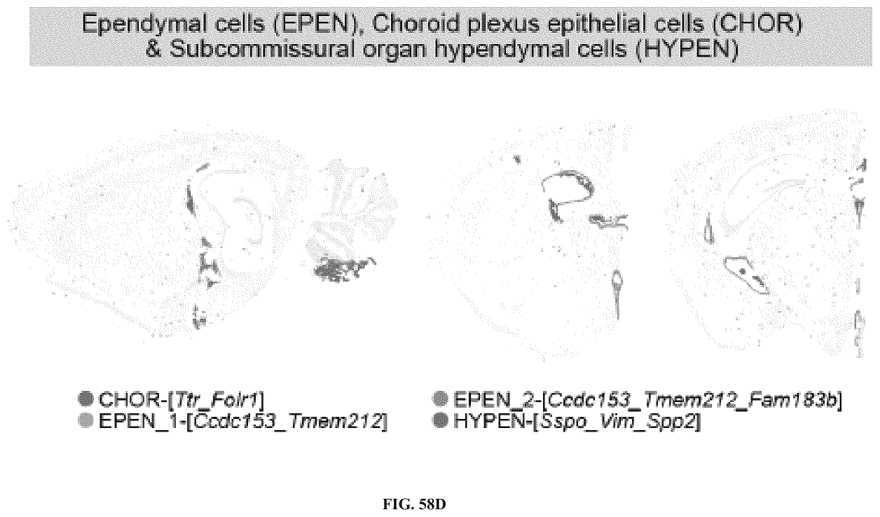

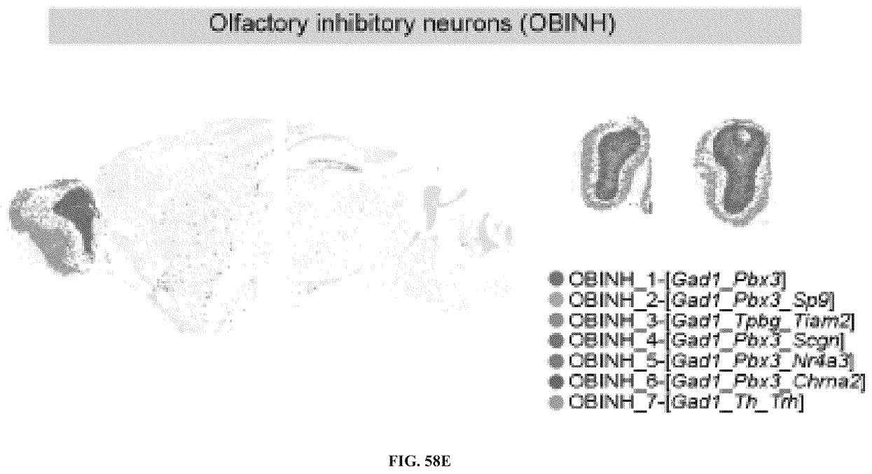

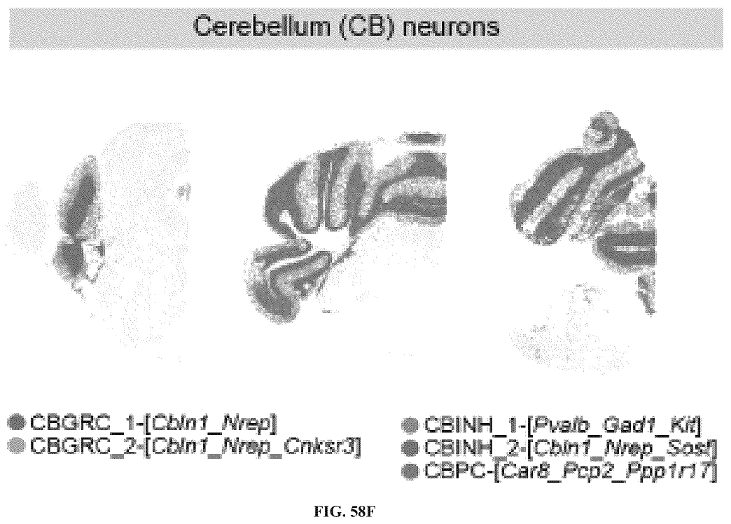

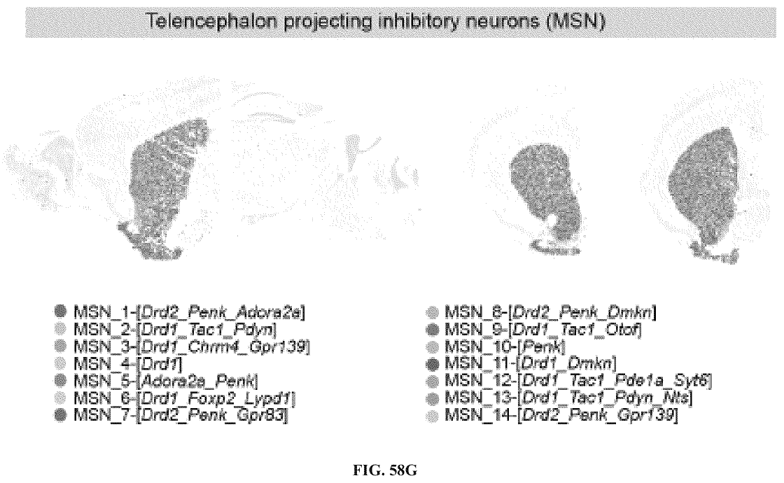

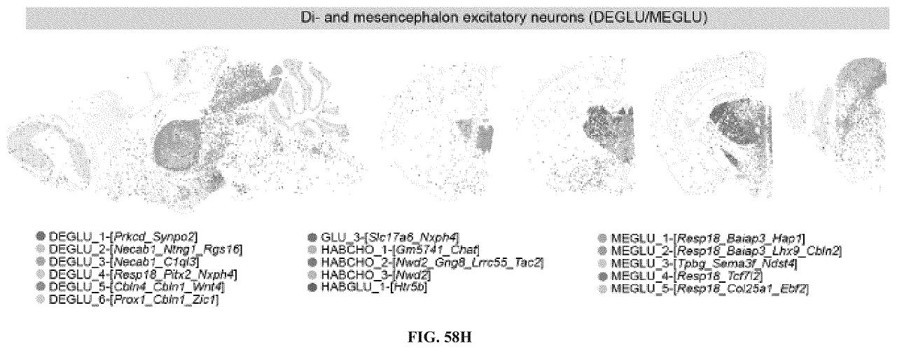

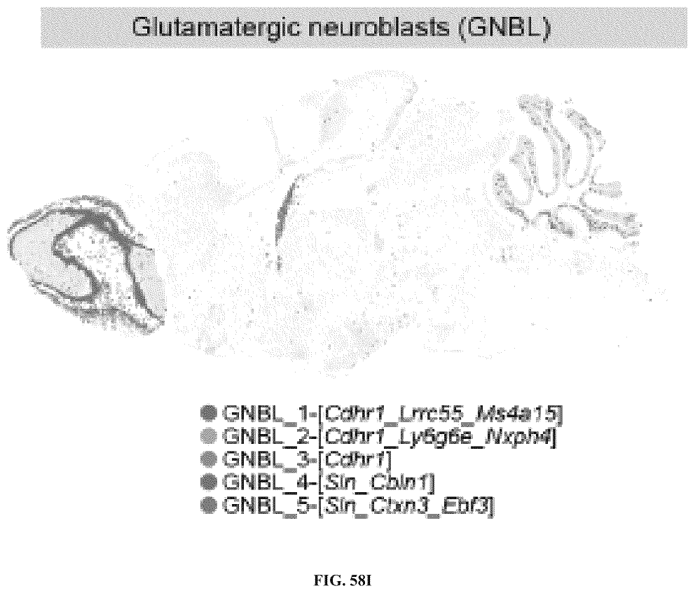

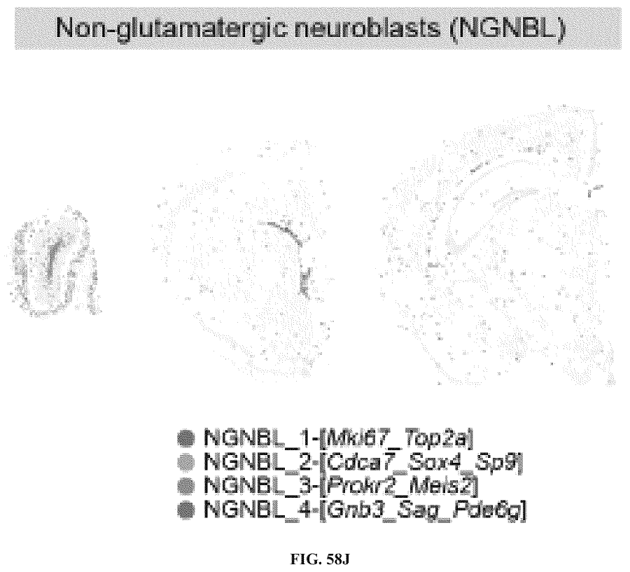

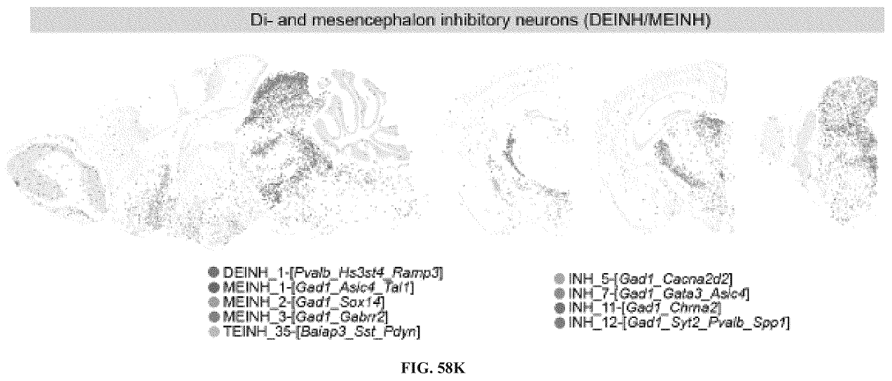

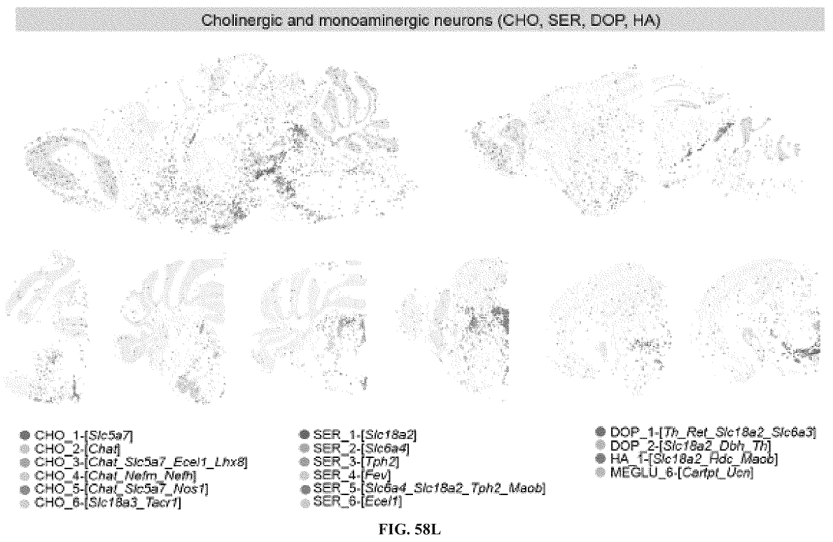

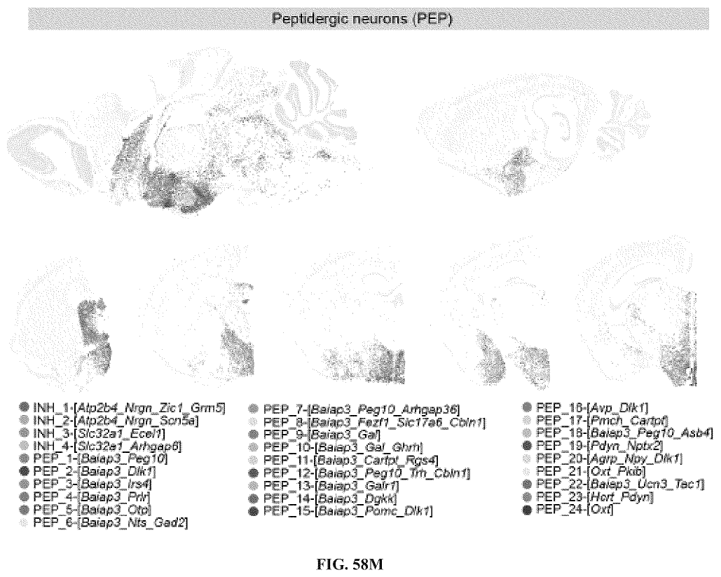

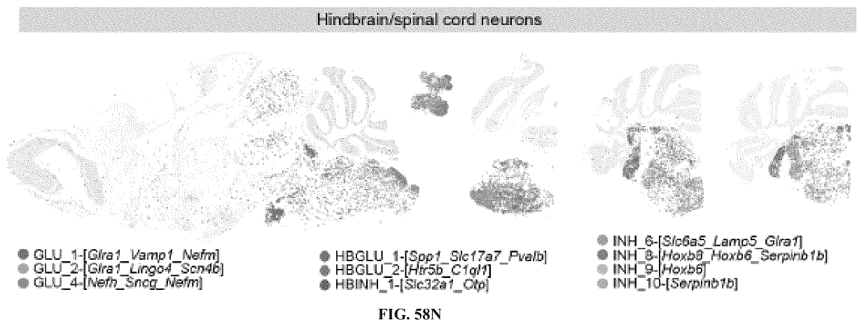

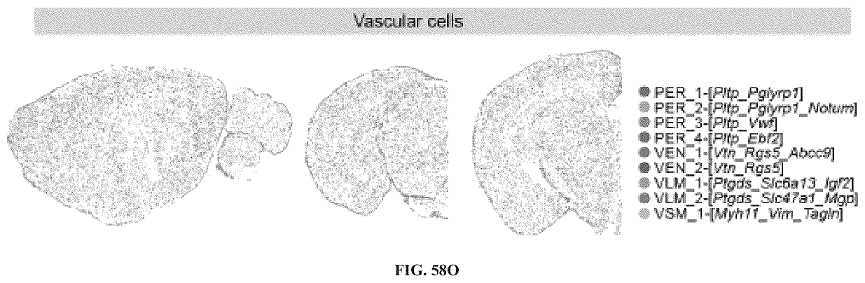

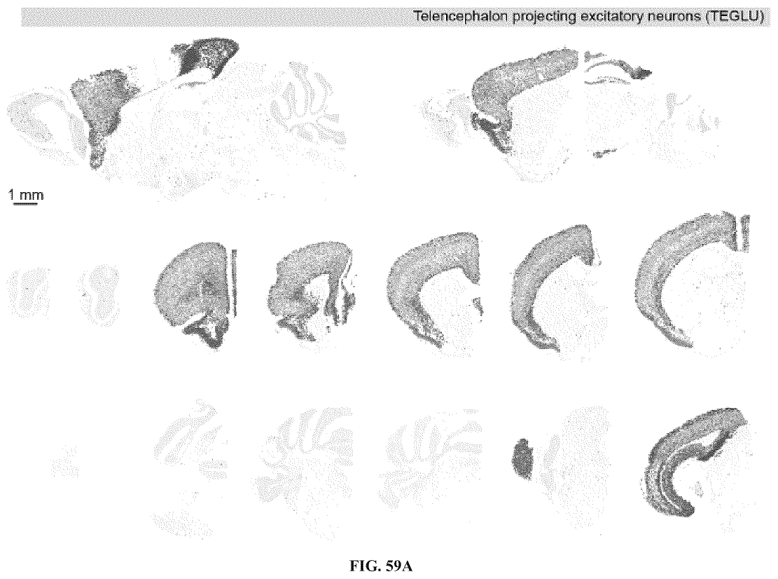

After batch correction, cells were pooled from all the tissue slices and cell typing was performed by hierarchically clustering single-cell expression profiles (.FIG. 57C). To annotate cell types and align with published cell type nomenclature, the data was integrated with an existing mouse CNS scRNA-seq atlas via Harmony (Korsunsky, I. et al. Nat. Methods 16, 1289-1296 (2019)). Leiden clustering followed by nearest neighbor label transfer identified 26 main cell types, including 13 neuronal, 7 glial, 2 immune, and 4 vascular cell clusters, all of which exhibited canonical marker genes and expected spatial distribution across the 20 tissue slices (FIGS. 51B, 57D-57E, 58A-58O, and 59A-59G). Further Leiden clustering within each main cluster resulted in 230 subclusters, including 190 neuronal, 2 neural crest-like glial, 13 CNS glial, 4 immune, and 9 vascular cell clusters (FIGS. 51B, 66B-D, 67A-67N, 68A and 68B). Each subcluster was annoted with symbols, cell counts, marker genes, and spatial distributions, and it was indicated whether they present cell types or states. Notably, the subcluster size in the data spanned approximately three orders of magnitude, ranging from abundant cell types such as oligodendrocytes OLG_1 (70,866 cells, 6.5% of total cells), to rare cell types such as Hdc⁺ histaminergic neurons HA_1 in the posterior hypothalamus (111 cells, 0.01% of total cells, FIGS. 58L, 59C, and 67I).

Molecularly defined, single-cell resolved cell type maps were then plotted across the adult mouse CNS (FIGS. 51C, 58A-58O, 59A, and 59B). The maps clearly delineated brain structures, including the cerebral cortex (41 telencephalon projecting excitatory neuron types, TEGLU; 34 telencephalon inhibitory interneuron types, TEINH), olfactory bulb (7 olfactory inhibitory neuron types, OBINH; olfactory ensheathing cells, OEC), striatum (14 telencephalon projecting inhibitory neuron types, MSN), cerebellum (5 cerebellum neuron types and astrocyte type AC_4), and brainstem (28 peptidergic neuron types, 16 cholinergic and monoaminergic neuron types, 16 di- and mesencephalon excitatory neuron types, DE/MEGLU, 9 di- and mesencephalon inhibitory neuron types, DE/MEINH, and 10 hindbrain and spinal cord neuron types), fully recapitulating the anatomical regions in the adult mouse CNS (FIG. 51C). Zooming in, these maps also revealed cell-type-specific patterns in fine tissue regions, such as the medial and lateral habenula, alveus, fimbria, and ependyma (FIG. 1D), with individual cells (FIG. 51E) and RNA molecules (FIG. 51F) fully resolved in space.

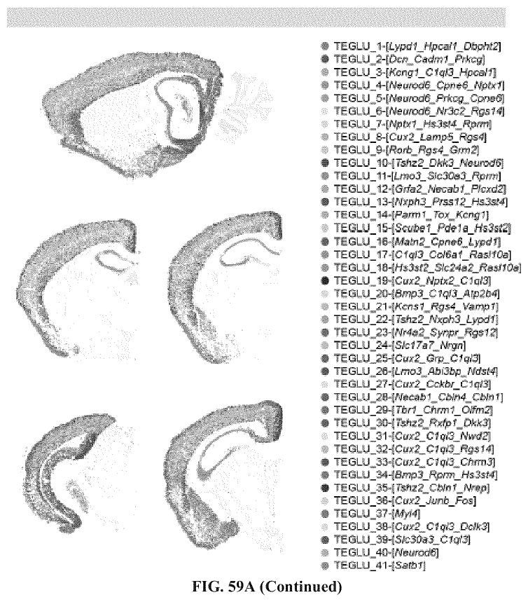

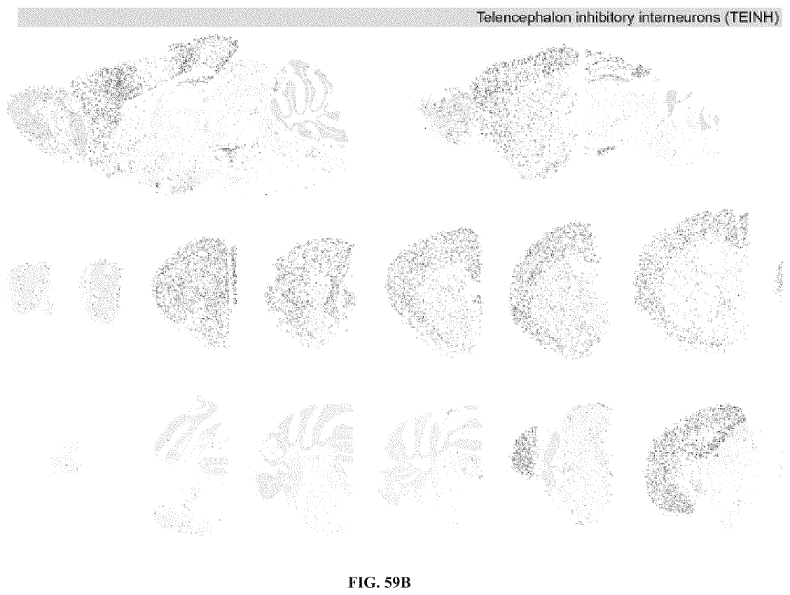

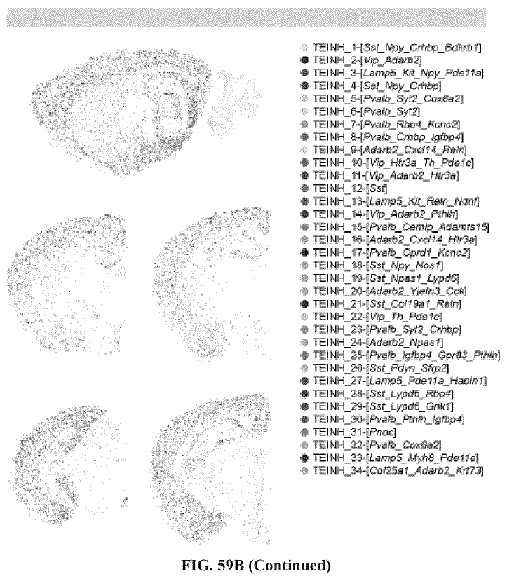

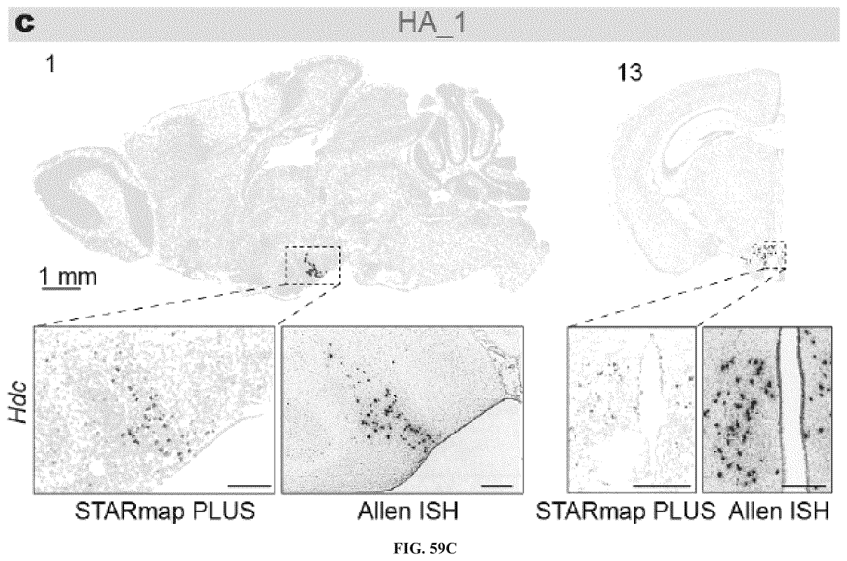

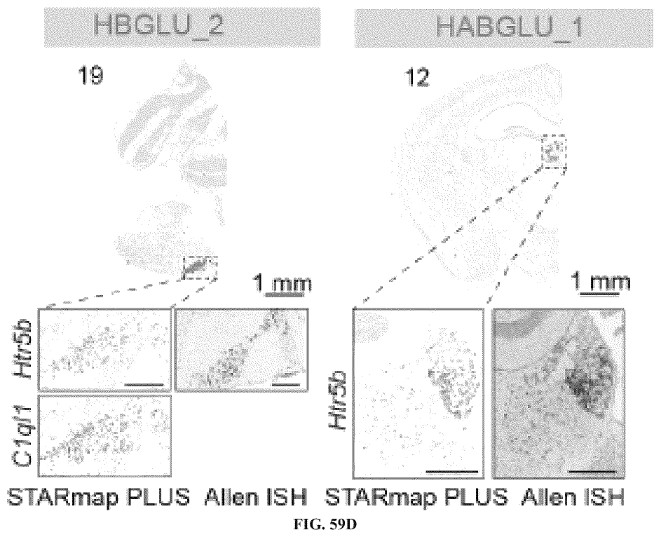

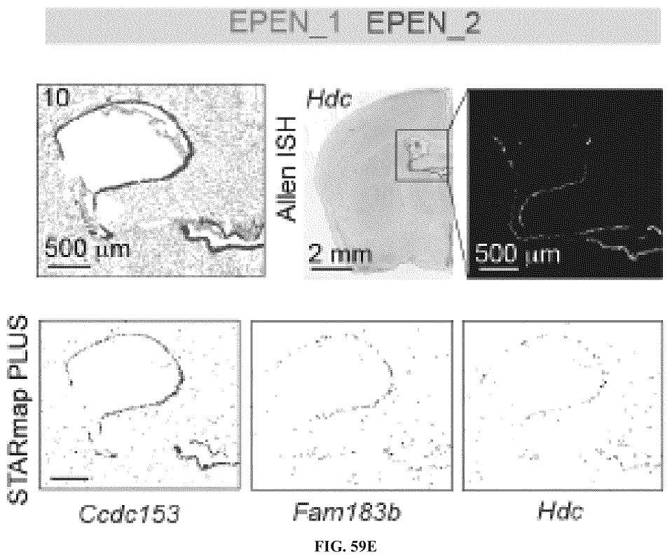

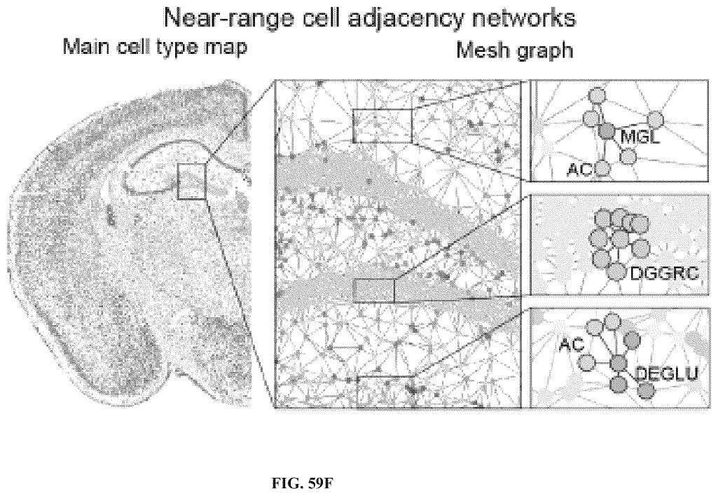

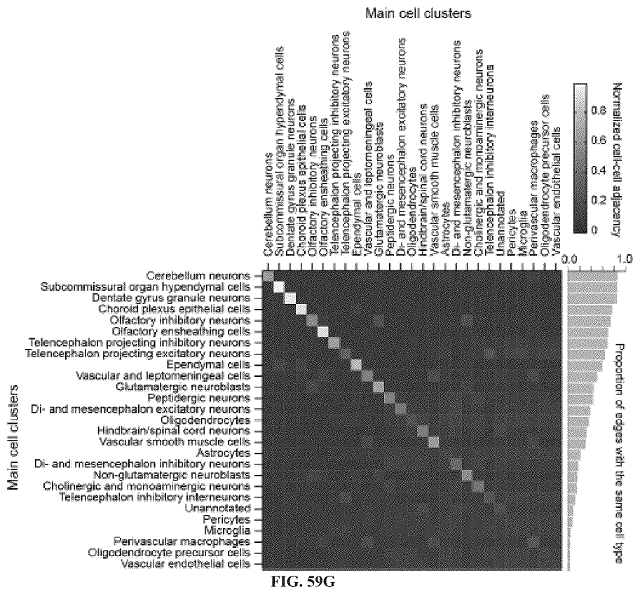

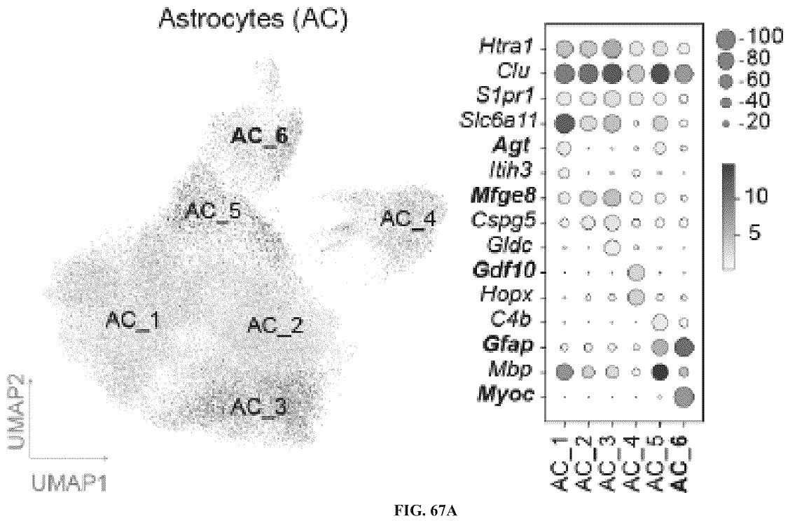

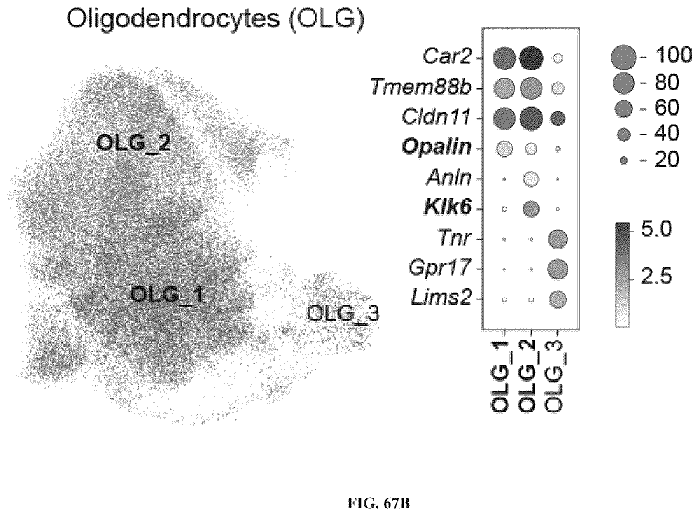

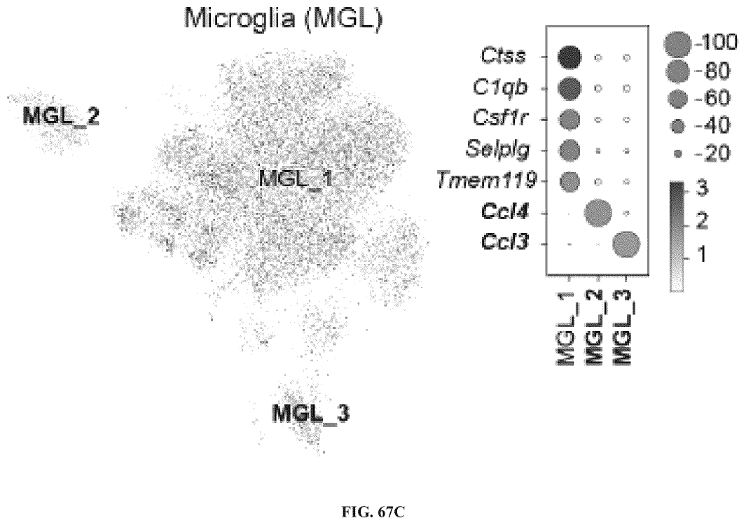

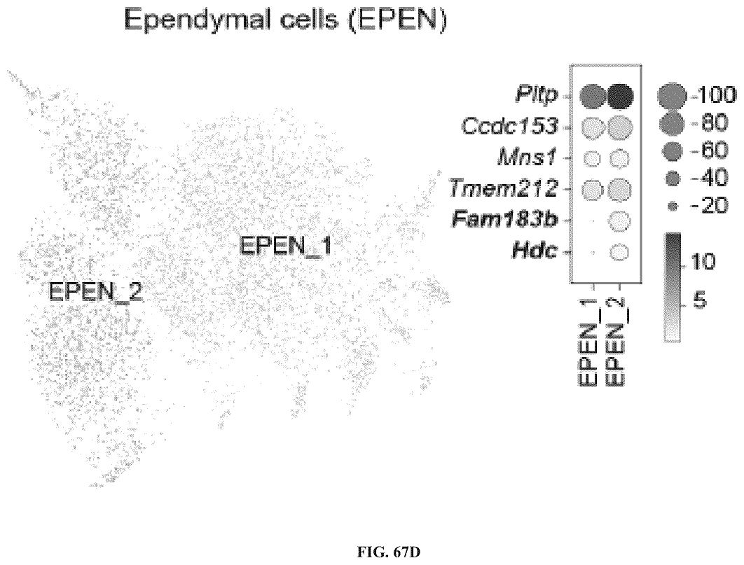

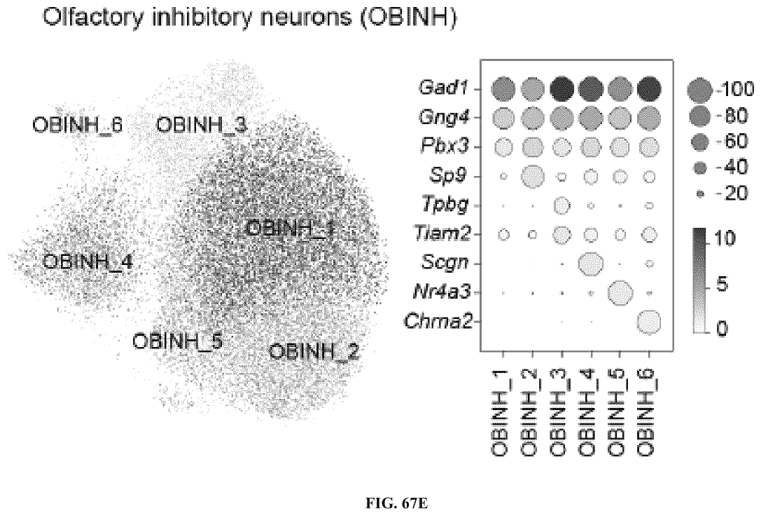

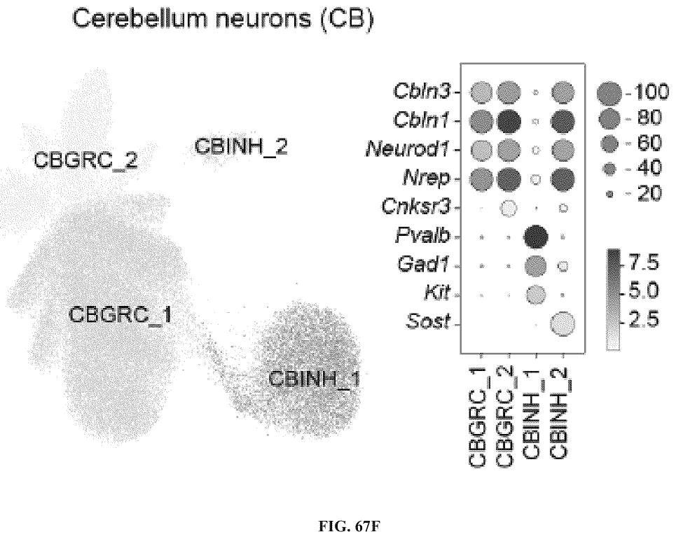

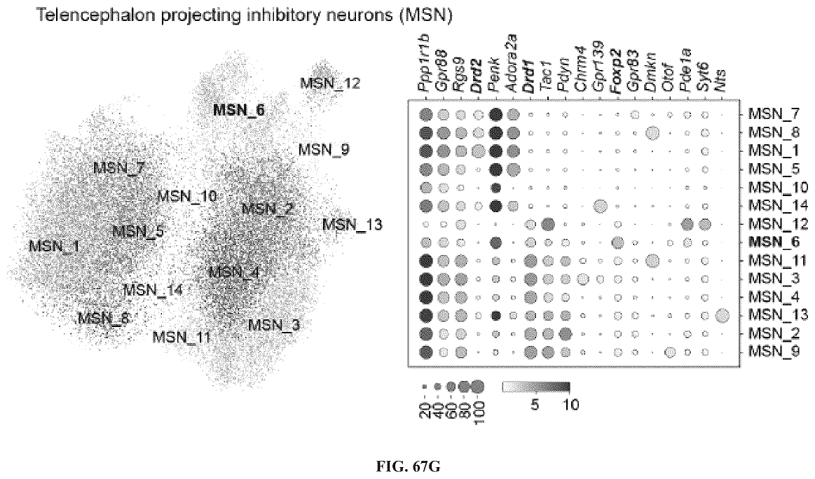

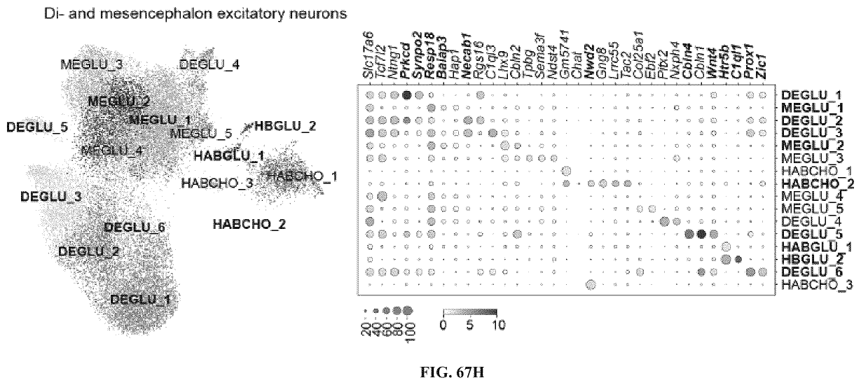

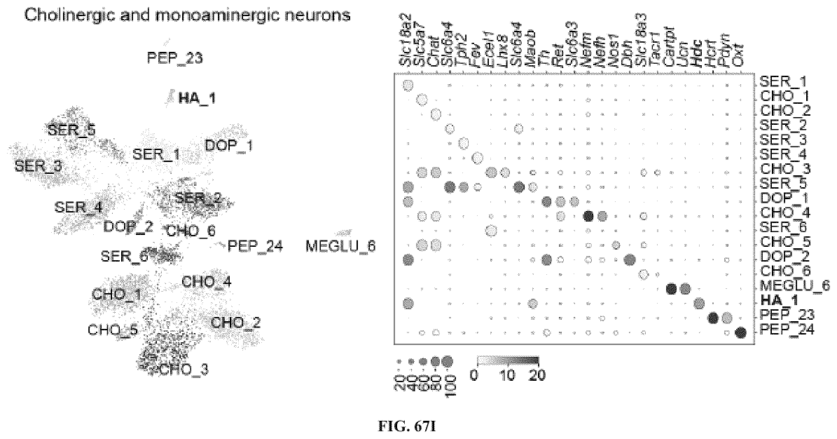

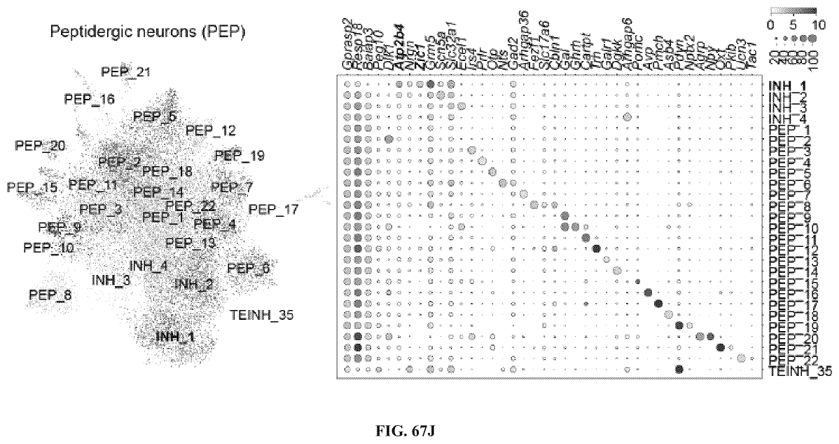

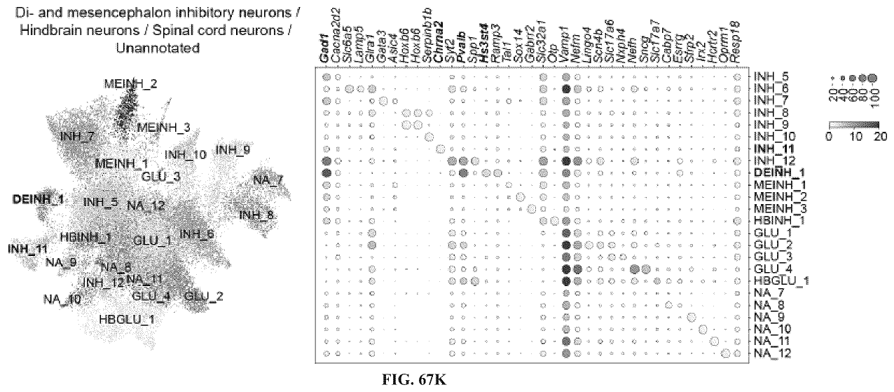

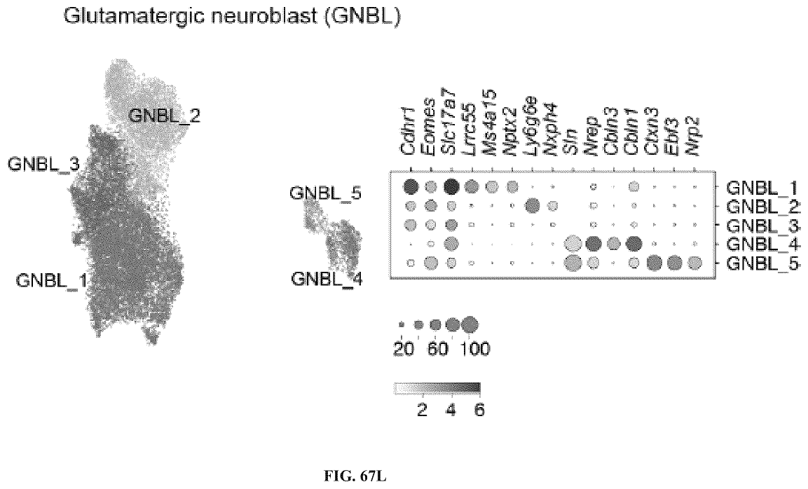

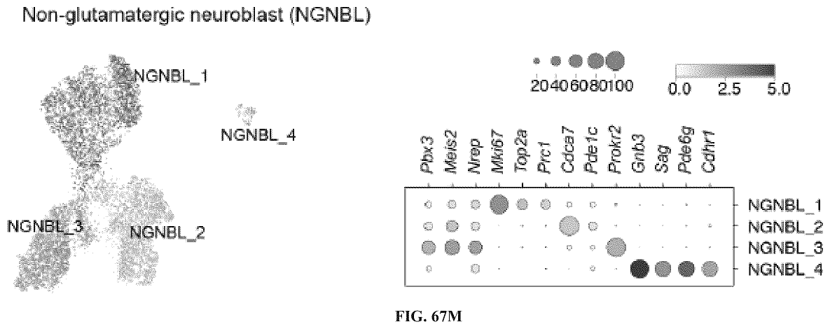

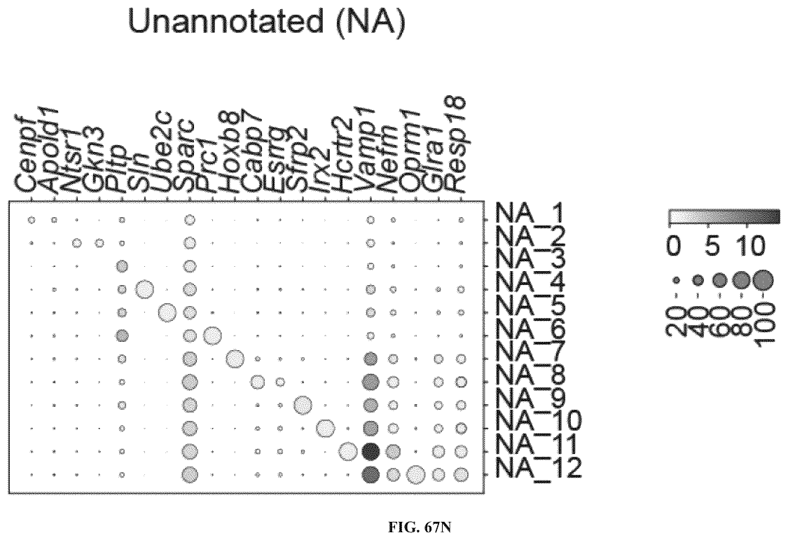

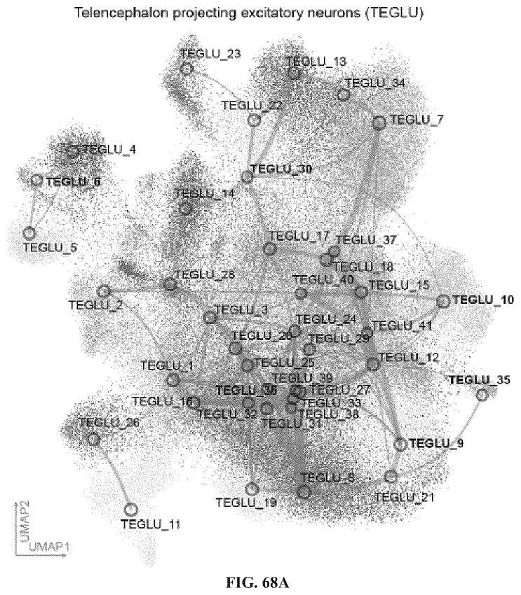

Remarkably, compared with previous scRNA-seq results, the molecular resolution, single-cell mapping across a large number of cells enabled more precise annotation of molecular cell types by their spatial distributions. For instance, in addition to the previously reported Htr5b⁺ neurons in the inferior olivary complex of the hindbrain (HBGLU_2, C1ql1⁺, 204 cells), another Htr5b⁺ cluster located in the habenula (HABGLU_1, C1ql1⁻, 318 cells) was identified (FIGS. 59D and 67H). It was also observed that ependymal cells contain two subclusters (EPEN_1, Ccdc153⁺; EPEN_2, Ccdc153⁺Fam183b⁺) with differential distributions across the medial-lateral axis (FIGS. 59E and 67D). Moreover, the single-cell-resolved molecular cell type maps allowed the examination of cell-cell adjacency across the entire brain (FIGS. 51E and 59F), revealing that neuronal cell types tend to form near-range networks with the same main cell type while glial and immune cell types are more sparsely distributed among other cell types (FIG. 59G). In brief, the molecular resolution, brain-wide in situ sequencing data provided substantial potential in annotating molecular cell types and characterizing cellular neighborhoods in space.

### Example 8.2: Molecularly Defined CNS Tissue Regions

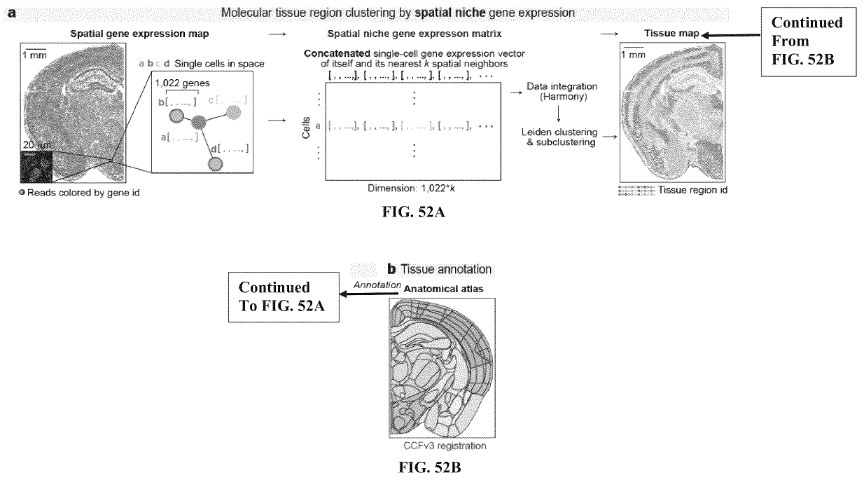

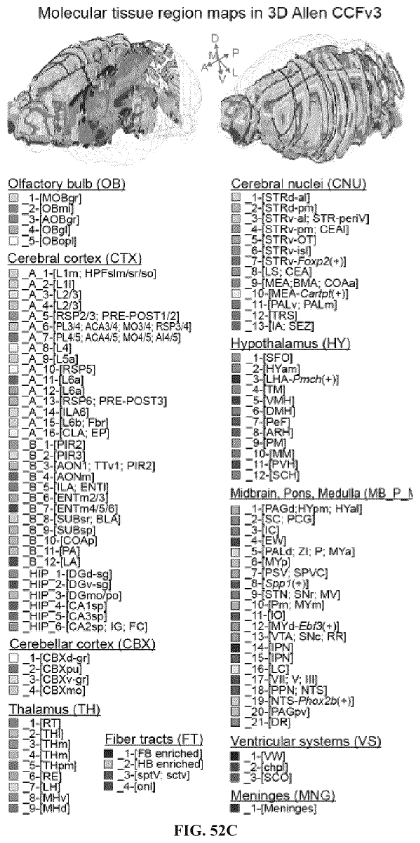

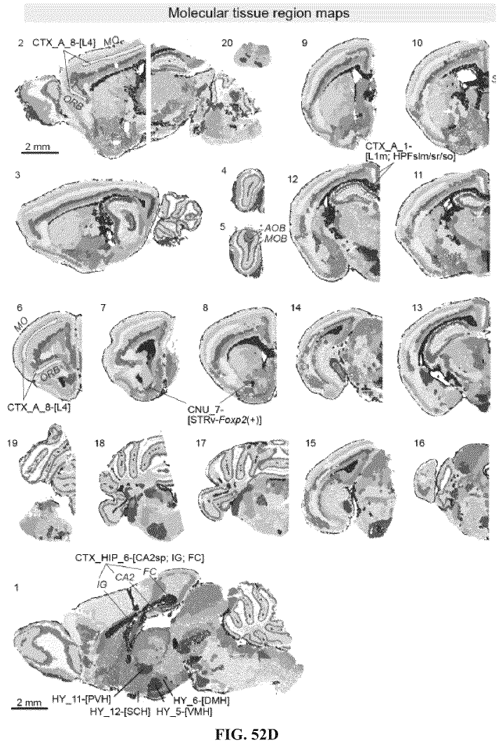

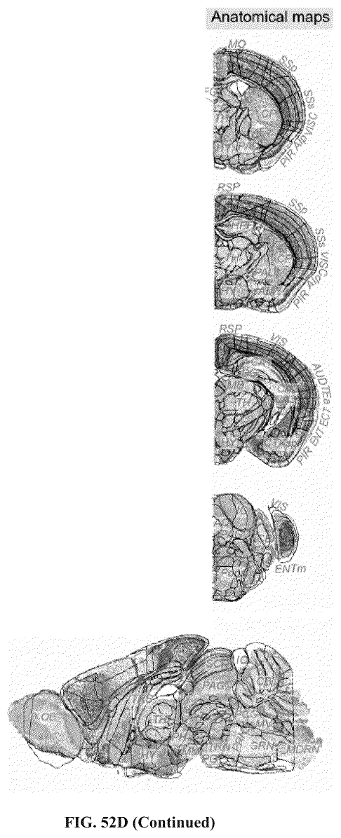

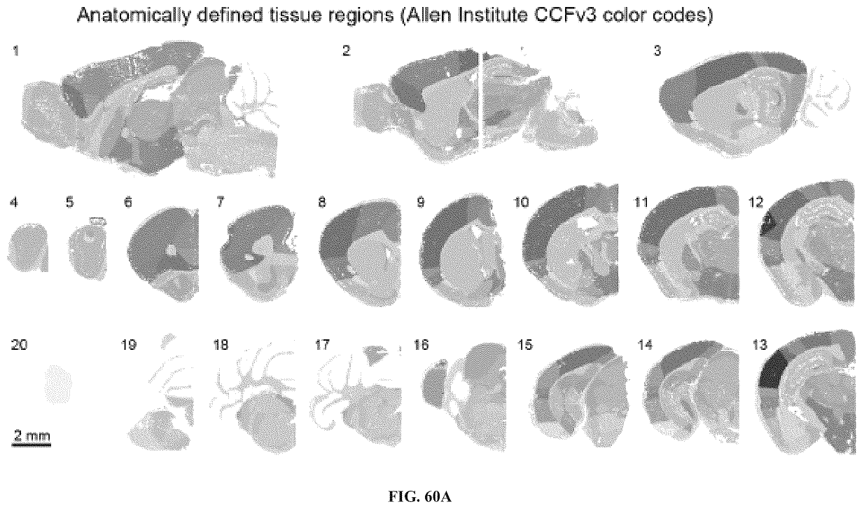

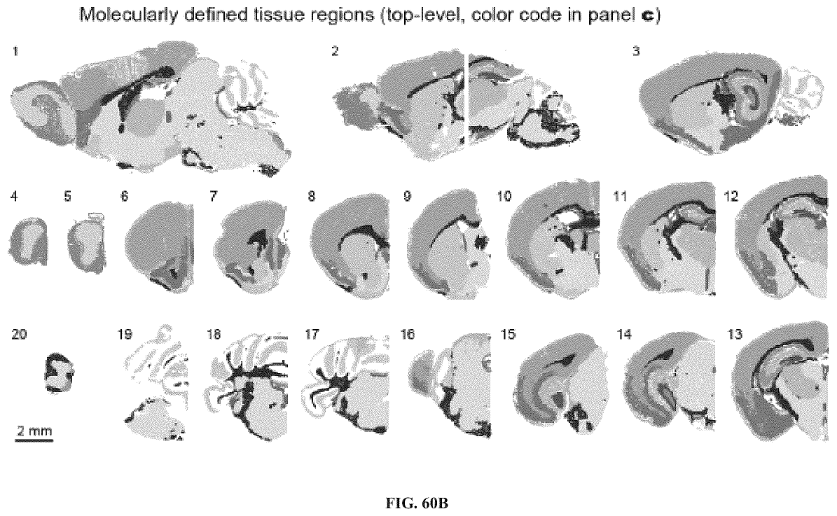

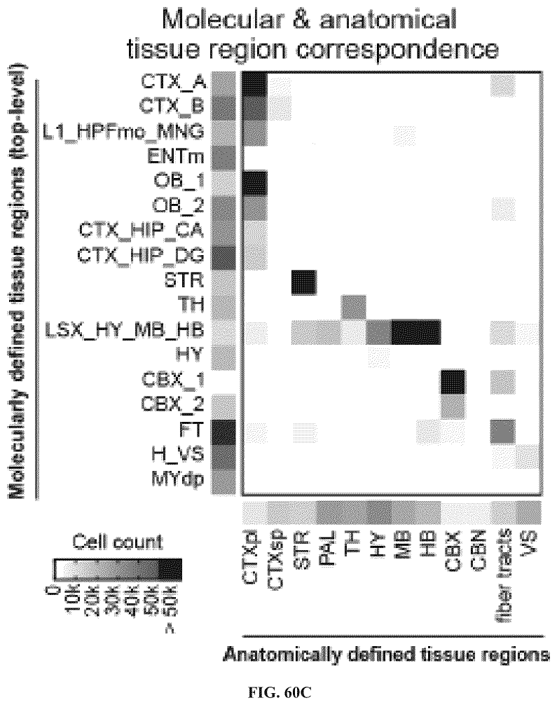

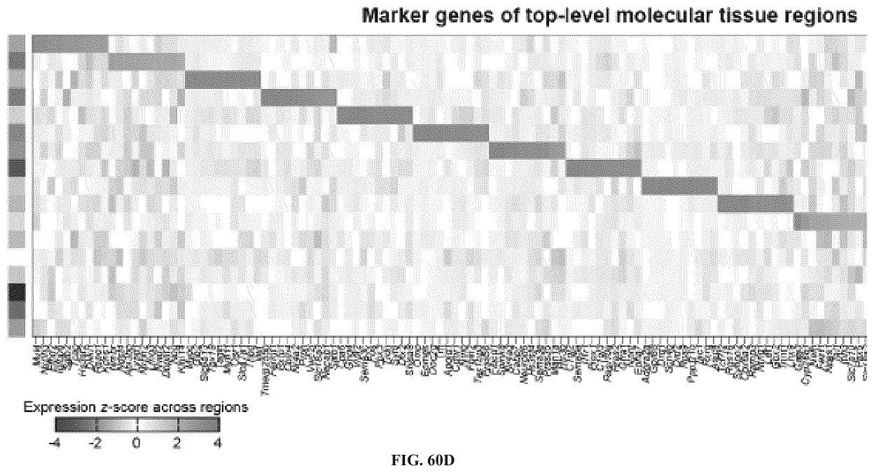

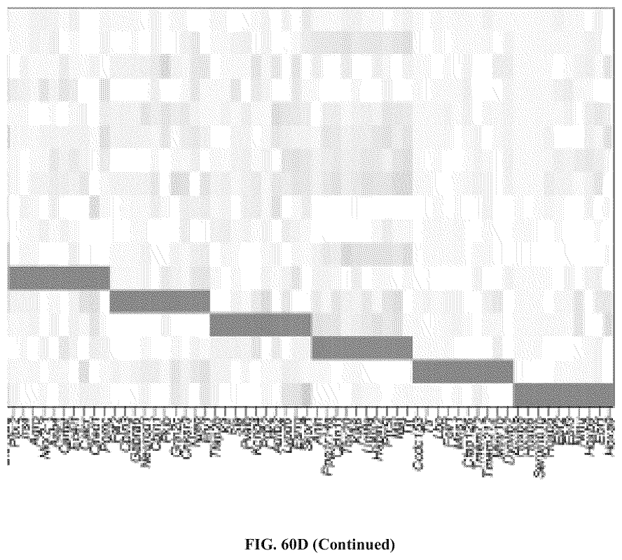

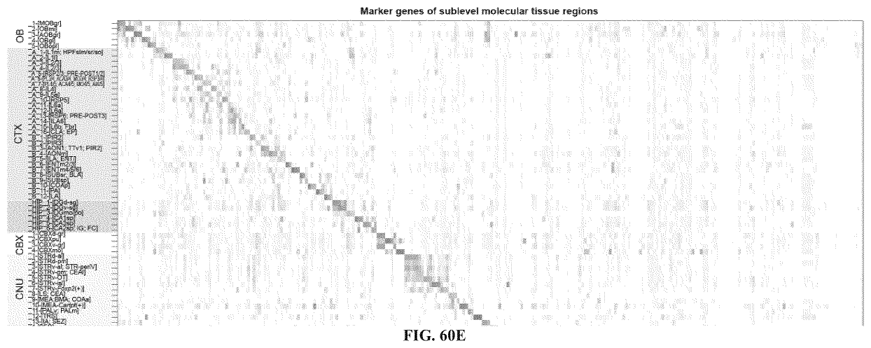

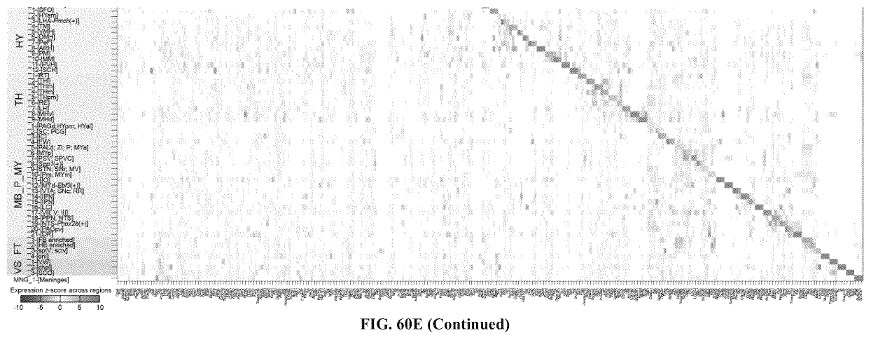

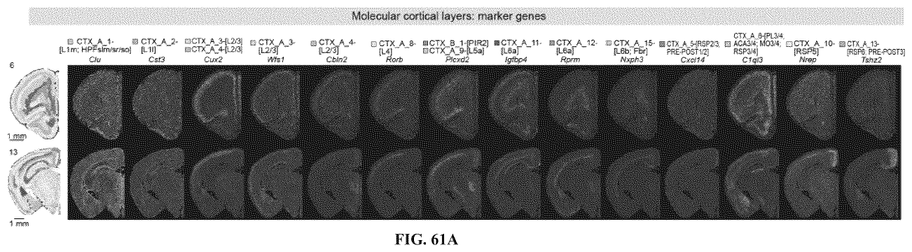

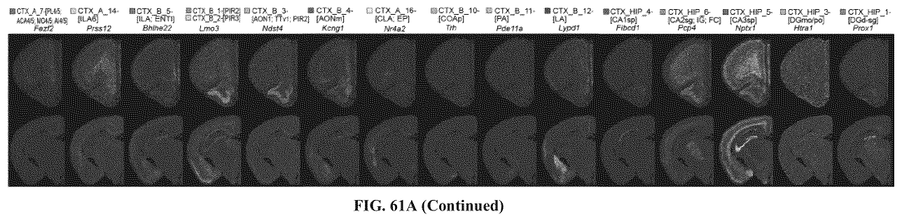

Next, molecularly defined tissue region maps were built directly from spatial niche gene expression profiles. Such data-driven identification of tissue regions provided systematic and unbiased molecular definitions of CNS tissue domains. Briefly, for a given tissue slice, a spatial niche gene expression vector of each cell was formed by concatenating its own single-cell gene expression vector and those of its k nearest neighbors (kNNs) in the physical space. The resulting spatial niche gene expression matrices for each slice were integrated and subjected to Leiden clustering (FIG. 52A) to identify major brain tissue regions (17 top-level clusters) and then subclusters within each major region (106 sublevel clusters). To compare and annotate the molecularly defined tissue regions with anatomically defined tissue regions, sample slices were registered into the established Allen Mouse Brain Common Coordinate Framework (CCFv3, FIGS. 52B and 52C) and labeled individual cells in the datasets with CCF (Common Coordinate Framework) anatomical definitions (FIG. 60A).

Overall, the molecularly defined tissue regions aligned well with the anatomically defined regions (FIGS. 52D and 60A-60C) and were annotated accordingly. First, the identified marker genes in each top-level molecular tissue region were consistent with region markers reported in the Allen In Situ Hybridization (ISH) database (FIG. 60D), such as molecular dentate gyrus (DG) marker C1ql2, molecular striatal marker Ppp1r1b, and molecular thalamic marker Tcf7l2. Next, the 106 sublevel clusters include 5 molecular olfactory bulb regions (OB_1˜5), 34 molecular cerebral cortex regions (CTX_A_1-16, CTX_B_1-12, and CTX_HIP_1-6), 13 molecular cerebral nuclei regions (CNU_1˜13), 4 molecular cerebellar cortex regions (CBX_1-4), 9 molecular thalamic regions (TH_1-9), 12 molecular hypothalamic regions (HY_1-12), 21 molecular tissue regions in the midbrain, pons, and medulla (MB_P_MY_1-21), 4 molecular fiber-tract regions (FT_1˜4), 3 molecular ventricular system regions (VS_1˜3), and the molecular meninges (MNG 1). Individual sublevel molecular tissue regions were subsequently annoted with symbols describing fine anatomical definitions, preferential distribution along body axes (anterior vs. posterior, medial vs. lateral, dorsal vs. ventral), or marker genes (FIG. 60E), following the anatomical nomenclature in the Allen Institute adult mouse atlas (FIG. 52D). For example, OB_1 corresponds to the granule layer of the main olfactory bulb and is thus named OB_1-[MOBgr].

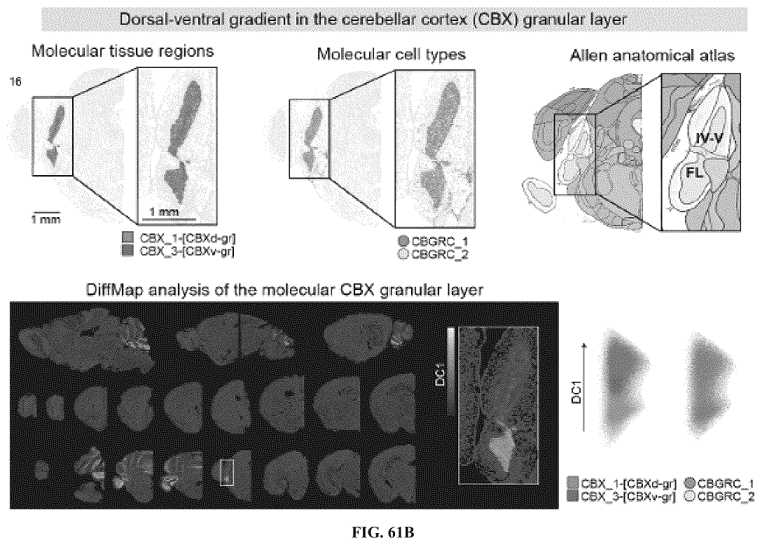

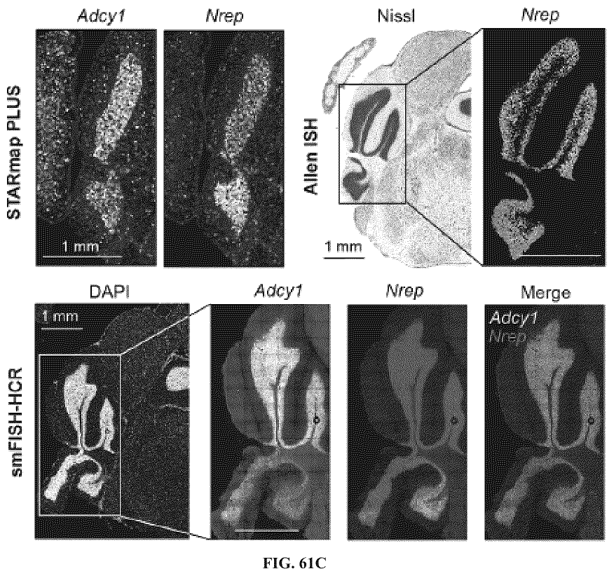

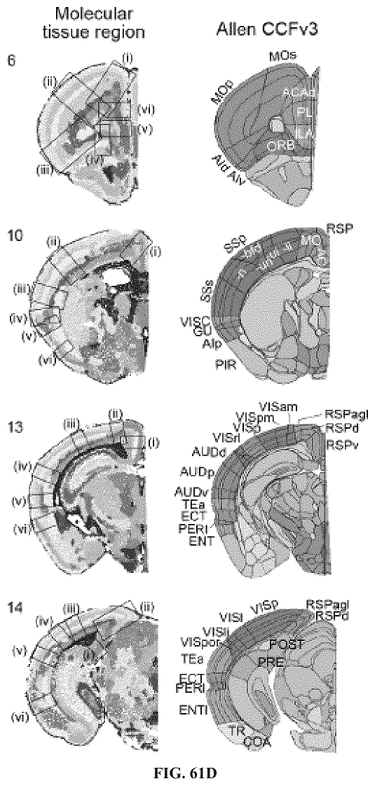

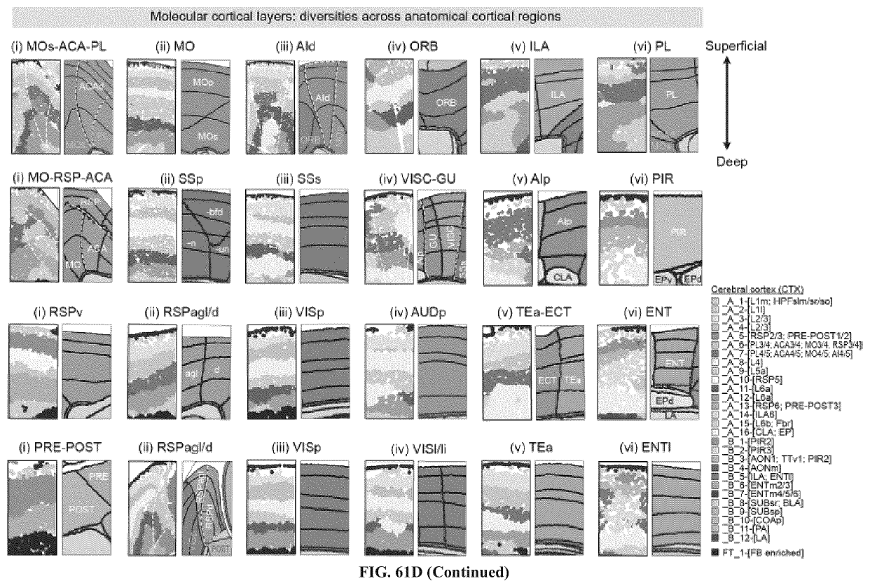

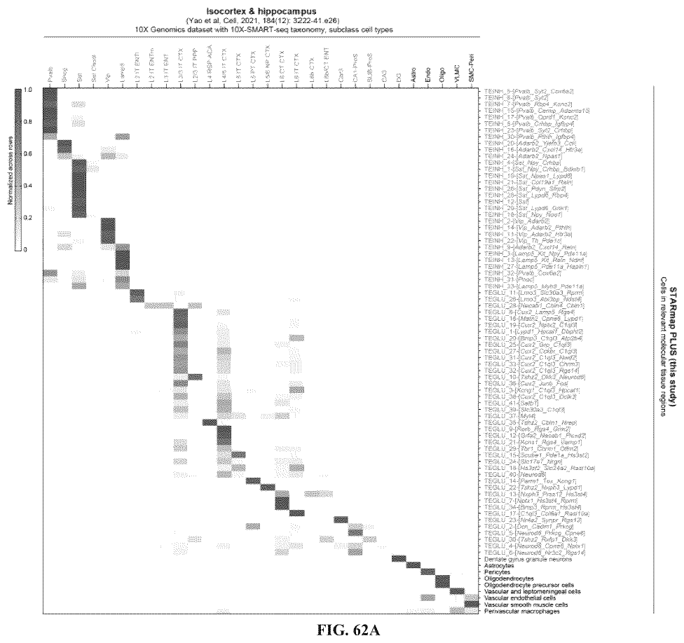

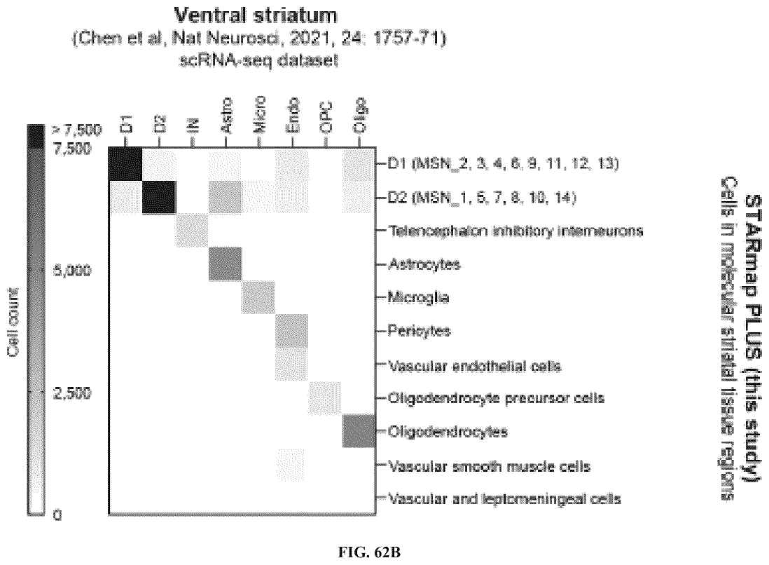

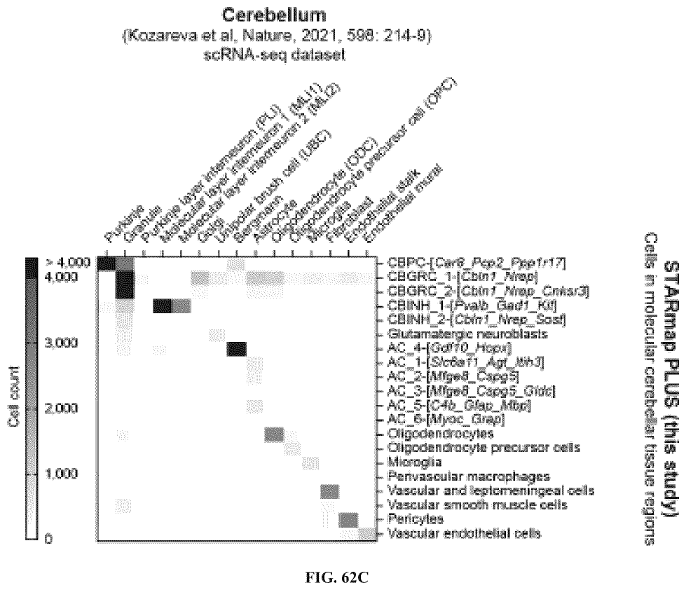

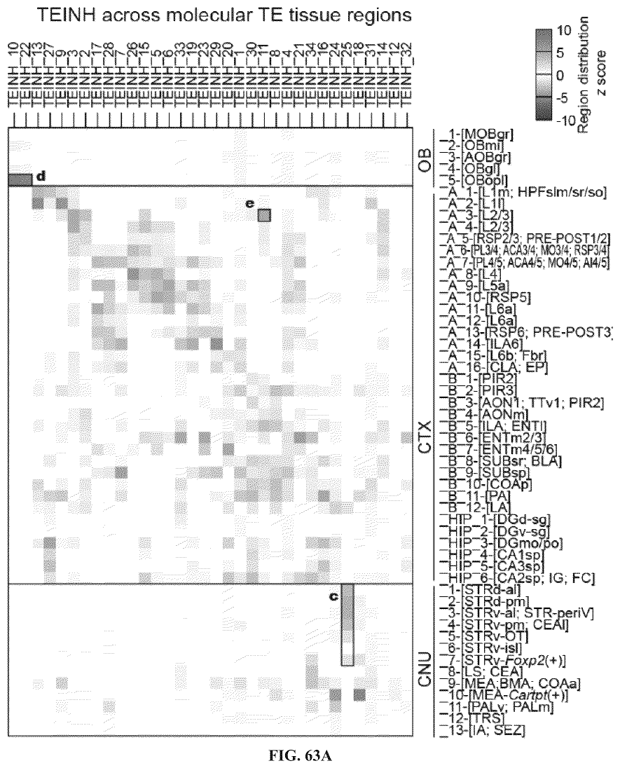

The molecular tissue annotation and marker genes were carefully examined by cross-referencing published studies and validating with smFISH-HCR™ (Choi, H. M. T. et al. Development 145, dev165753 (2018)) (single-molecule fluorescence in situ hybridization with hybridization chain reaction amplification). First, the molecular cerebral cortical regions resembled the laminar organization of anatomical cortical layers and recapitulated layer-specific markers (e.g., Cux2 in CTX_A_3-[L2/3] and CTX_A_4-[L2/3], Rorb in CTX_A_8-[L4], Plcxd2 in CTX_A_9-[L5a], and Rprm in CTX_A_12-[L6a]; FIGS. 52D and 61A). Second, in the hippocampal region, expected markers for individual Ammon's hom field pyramidal layers were observed, including Fibcd1 in CTX_HIP_4-[CA1sp], Pcp4 in CTX_HIP_6-[CA2sp; IG; FC], and Nptx1 in CTX_HIP_5-[CA3sp](FIG. 61A and FIG. 52D slices 1-3, 11-15). Third, both molecular olfactory bulb regions (OB_1˜5) and molecular cerebellar cortical regions (CBX_1˜4) formed delicate layered structures corresponding to anatomically defined layers (FIG. 52D, OB: slices 1-2, 4-5; CB: slices 1-3, 16-19). Notably, molecular tissue regions further reveal gene expression differences between the granule layers of the main and accessory OB (OB_1-[MOBgr] vs. OB_3-[AOBgr], marked by Inpp5j and Trhr, respectively; FIG. 52D, slice 5) and between the dorsal and ventral gradients within the CBX granular layer (CBX_1-[CBXd-gr] vs. CBX_3-[CBXv-gr], marked by Adcy1 and Nrep, respectively; FIG. 52D, slices 1-3, 16-19; FIGS. 61B and 61C). Fourth, multiple subdivisions of the molecular regions in thalamus (TH) and hypothalamus (HY) appeared as spatially segregated nuclei, corresponding to anatomically defined structures distributed along body axes (FIG. 52D, slices 1, 11-13), such as the Six3(+) reticular nuclei of thalamus (TH_1-[RT]), the Spon1(+) nucleus reunions of thalamus (TH_6-[RE]), the Chrna3(+) ventral medial habenula (TH_8-[MHv]), the Fezf1(+) ventromedial hypothalamic nucleus (HY_5-[VMH]), the Oxt(+) paraventricular hypothalamic nucleus (HY_11-[PVH]), the Ppp1r17(+) dorsal medial hypothalamus (HY_6-[DMH]), the Agrp(+) arcuate hypothalamic nucleus (HY_8-[ARH]), and the Prokr2(+) hypothalamic suprachiasmatic nucleus (HY_12-[SCH]) (FIGS. 52D and 60E). Finally, in the midbrain and hindbrain, gene signatures in fine structures of brain nuclei were captured, such as Cartpt in the Edinger-Westphal nucleus (MB_P_MY_4-[EW]), Dbh in the locus coeruleus (MB_P_MY_16-[LC]), and Chrna2 in the molecular apical interpeduncular nucleus (MB_P_MY_14-[IPN]) (FIGS. 62D and 60E).

However, molecularly defined tissue regions are not necessarily the same as anatomically defined tissue regions. On the one hand, molecular tissue regions illustrate molecular spatial heterogeneity that lacks obvious anatomical borderlines. For example, the molecular cortical layer maps revealed the similarity and differences in molecular layer compositions among various cortical regions across the medial-lateral and anterior-posterior axes (FIGS. 52D and 61D). Specifically, previous studies have indicated a putative cortical layer 4 (L4) in the motor cortex, whose existence was supported by the molecular tissue regions (CTX_A_8-[L4], marked by Rorb and Rspo1). It was further uncovered that L4 also exists in the orbital cortex (ORB) (FIG. 52D slices 2, 6). Additionally, previous studies have identified atypical Foxp2+D1 MSN cell types in the striatum. The data further illustrated a unique molecular tissue region (CNU_7-[STRv_Foxp2(+)]) that contains Foxp2+D1 MSNs and forms patch-like structures at the boundary of the ventral striatum (FIG. 52D, slices 8-11, 2-3). On the other hand, molecular tissue regions revealed spatial gene expression similarities among multiple anatomically defined regions. For example, the data suggest similar spatial expression profiles in the medial cortical layer 1 and hippocampal molecular layers (CTX_A_1-[L1m; HPFslm/sr/so], FIG. 52D), likely related to the homologous developmental origins of the isocortex and allocortex. As another example, indusium griseum (IG) and fasciola cinerea (FC) are two small subregions in the hippocampal region. Given their similarity in cytoarchitecture to the dentate gyrus (DG), whether they constitute unique subregions or belong to DG is still under debate. The molecular tissue regions suggested that, with respect to spatial gene expression, both IG and FC exhibit high resemblance with CA2 (CTX_HIP_6-[CA2sp; IG; FC], high in Rgs14 and Cabp7; FIG. 52D, slices 1, 8, 11-12), supporting the observed similarity among CA2, IG, and FC in the expression of key proteins, but precluding that they are remnants of the DG.

Collectively, a resource of molecular tissue regions across the entire mouse CNS registered with brain anatomy and annotated with region-specific marker genes was developed. The general match of molecular and anatomical tissue regions confirmed the molecular basis of mouse brain anatomy. More importantly, this unbiased identification of molecular tissue regions allowed for the discovery of new tissue architectures that complement the established brain anatomy, as further illustrated in a subsequent joint analysis of molecular cell types and tissue regions.

### Example 8.3: Joint Molecular Cell Types and Regions

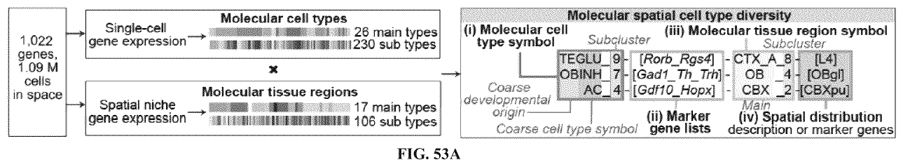

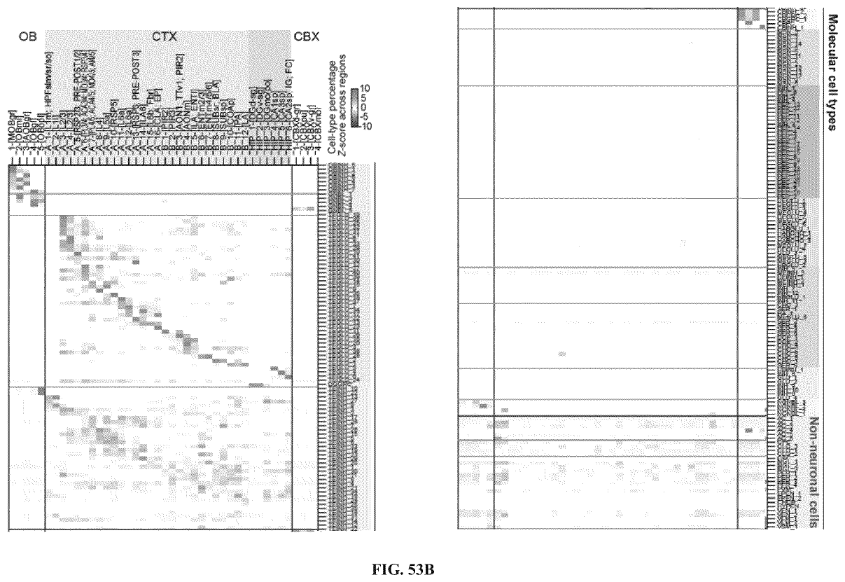

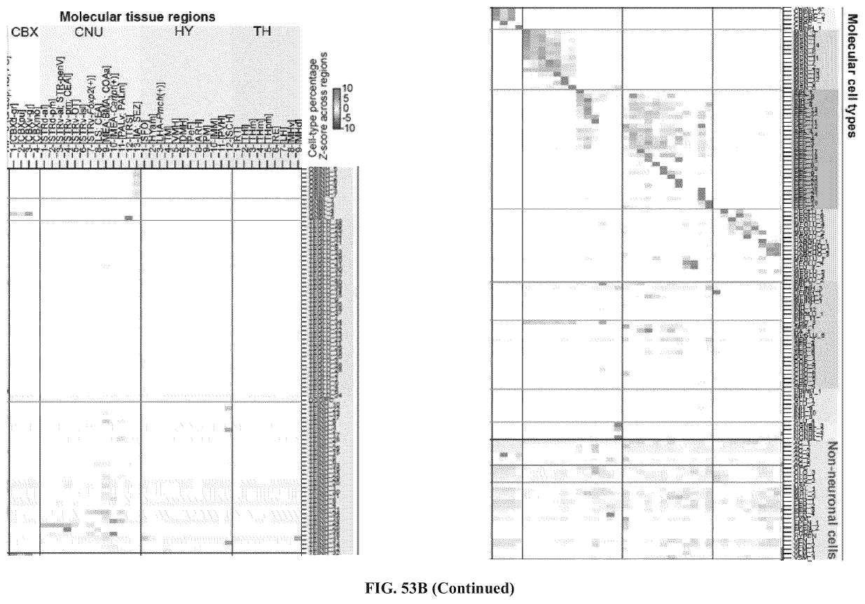

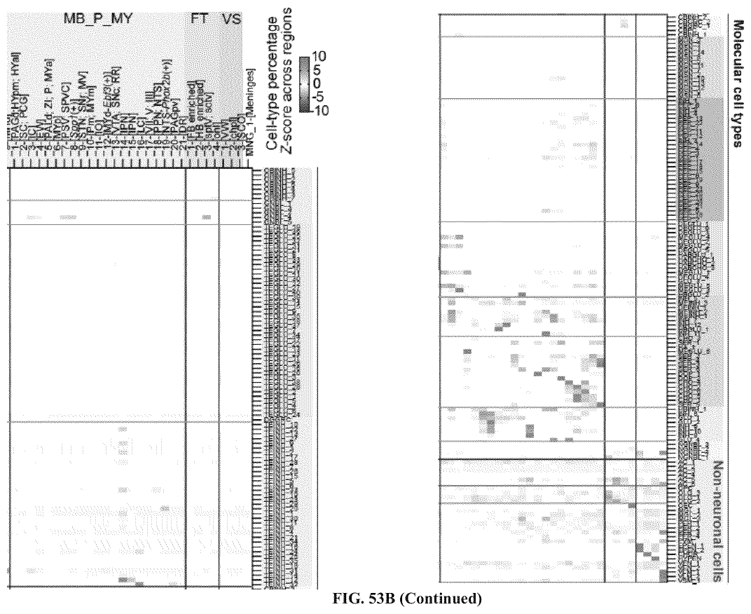

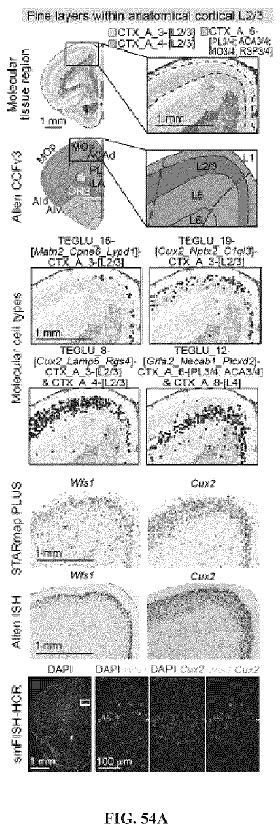

A comprehensive molecular spatial cell type nomenclature was then created by combining molecular cell type, subtype, marker genes, and molecular tissue region distribution information for each cell (FIG. 53A), resulting in 1,997 molecular spatial cell types. This joint definition enabled the further validation of the annotated molecular cell types by cross-referencing scRNA-seq studies on subregions of the adult mouse brain. Indeed, good correspondence between the cell clusters and neuronal and glial cell types was observed in regional scRNA-seq results of the isocortex and hippocampus, ventral striatum, and cerebellum (FIGS. 7A-7C).

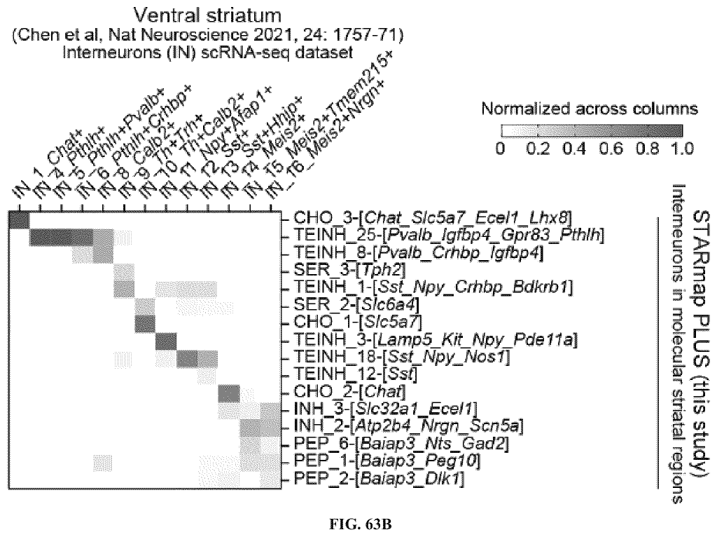

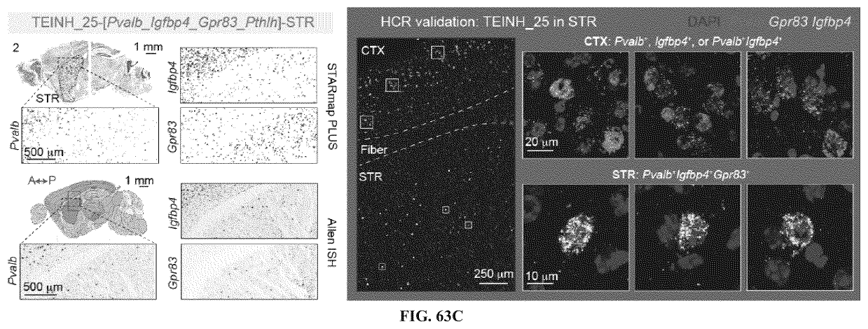

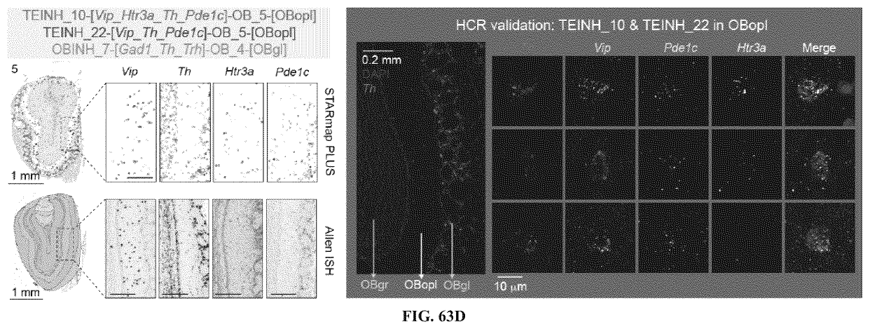

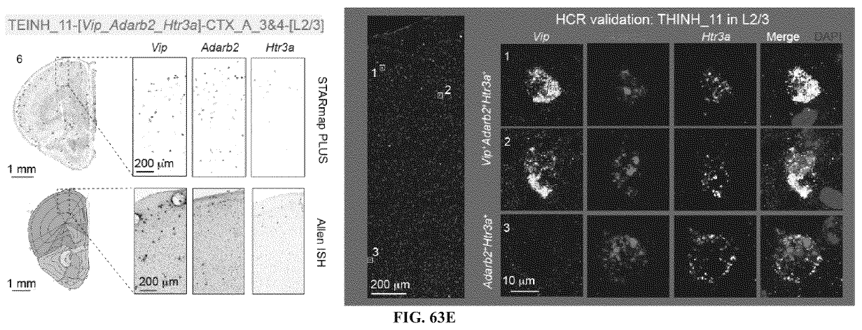

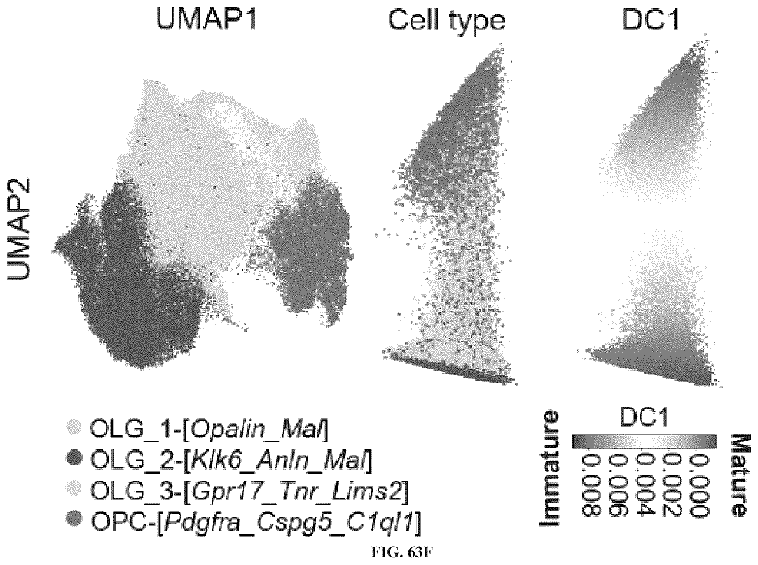

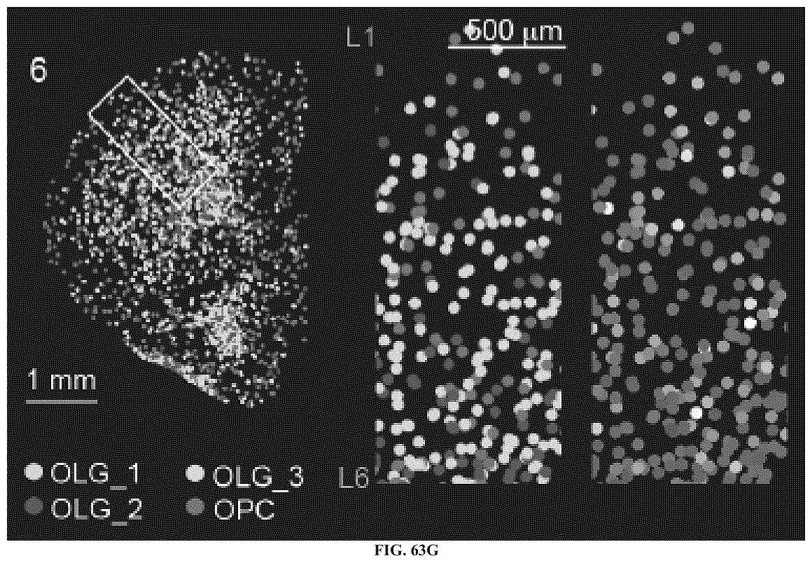

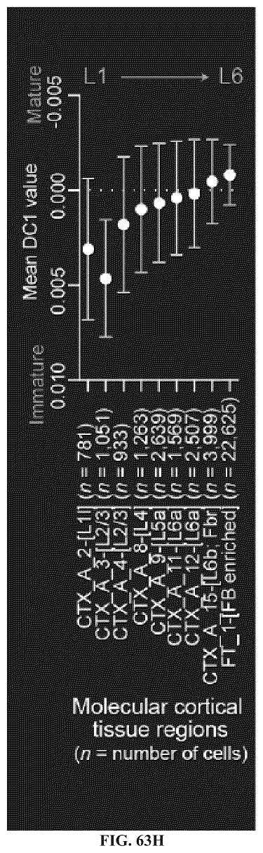

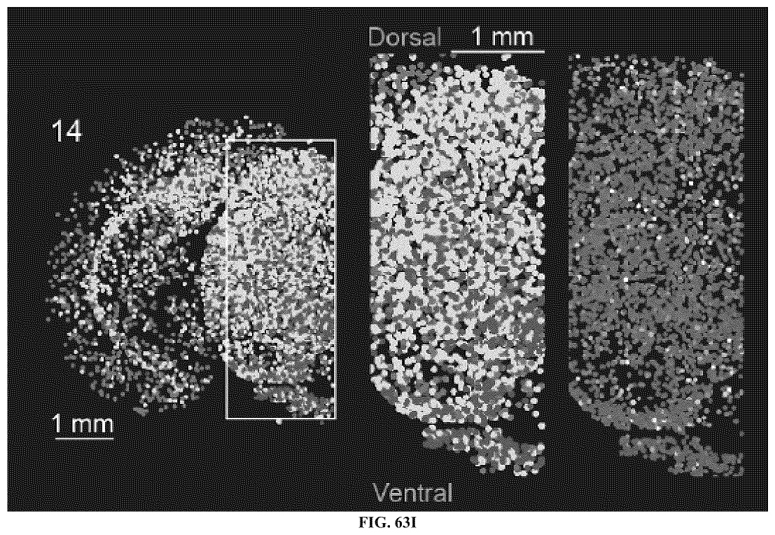

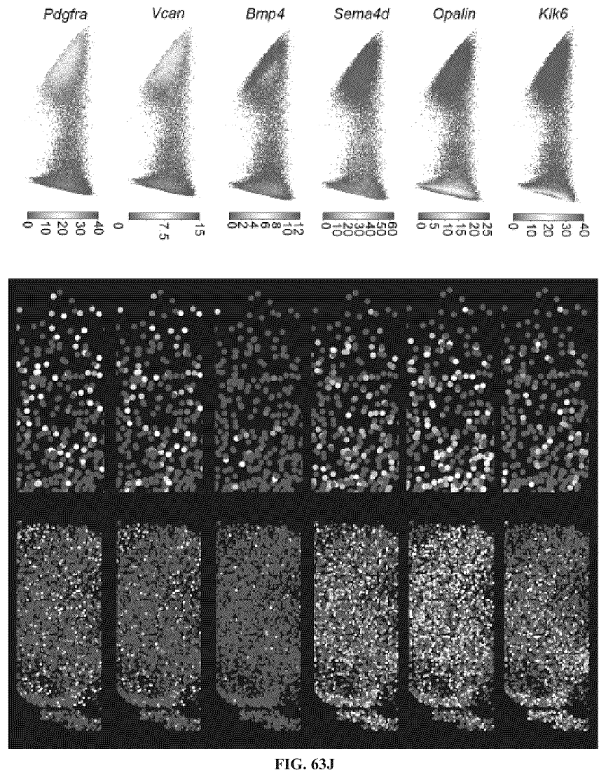

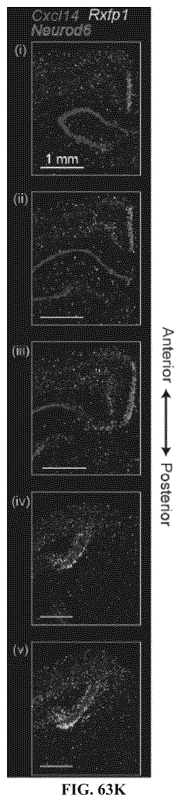

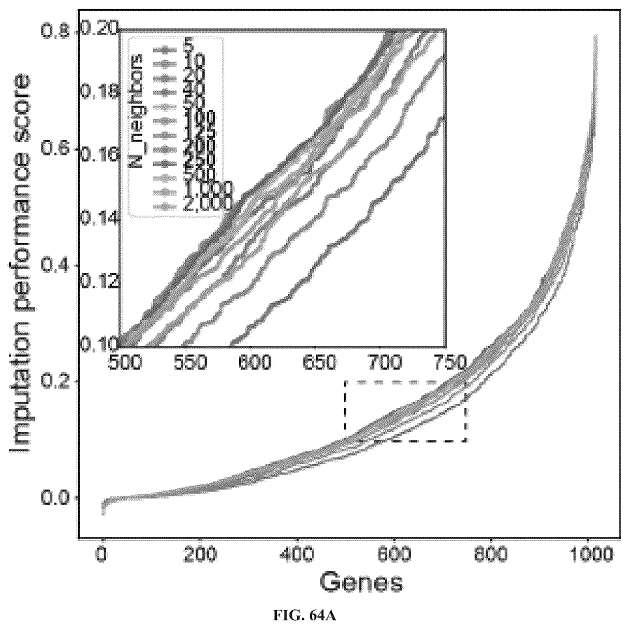

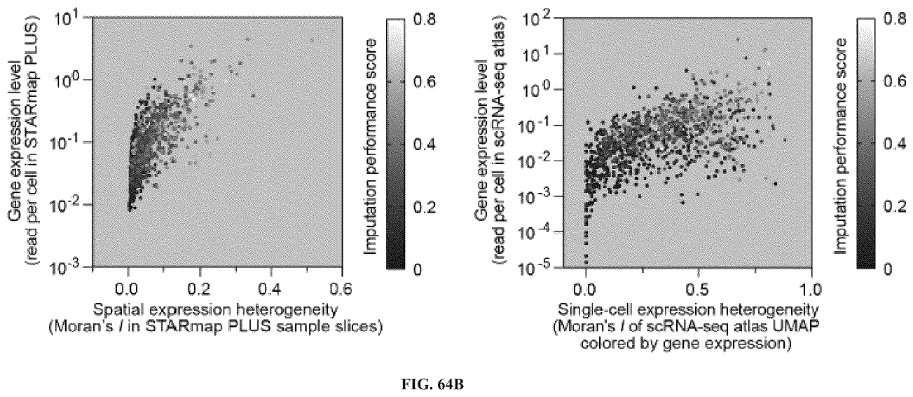

Using these spatially resolved cell type labels, the spatial distribution of cell types across brain regions was systematically examined (FIG. 53B). In the cerebral cortex, a strong layer-specific distribution of projecting excitatory neurons (TEGLU) was observed (FIG. 53B). In addition, the data showed that modest layer preference of inhibitory interneurons (TEINH) exists across cortical areas (FIG. 53B) beyond previously reported primary visual cortex and primary motor cortex. The data also revealed new region-specific TEINH subtypes (FIG. 63A), which were further verified through smFISH-HCR™ as follows. the following were identified and experimentally validated (i) a striatum-specific intemeuron subtype, TEINH_25-[Pvalb_Igfbp4_Gpr83 Pthlh], which has been indicated in a previous single-cell RNA-seq study comparing cortical and striatal interneurons and a recent striatum scRNA-seq dataset (FIGS. 63B-63C); (ii) two Th⁺Vip⁺ interneuron subtypes, TEINH_10-[Vip_Htr3a_Th_Pde1c] and TEINH_22-[Vip_Th_Pde1c], which are restrictively located in the outer plexiform layer of the olfactory bulb (OB_5-[OBopl]) (FIGS. 63A and 63D) and distinct from the previously identified olfactory glomerular layer Th⁺Vip⁻ interneurons (OBINH_7-[Gad1_Th_Trh]); and (iii) a L2/3 enriched subtype TEINH_11-[Vip_Adarb2_Htr3a](FIGS. 63A and 63E). Furthermore, many neuronal cell types outside the cerebral cortex also exhibit defined spatial patterns (FIGS. 53B and 58A-58O). Differential distributions of olfactory inhibitory neuron (OBINH) cell types were observed across the layers in the olfactory bulb, and glutamatergic neuroblasts (GBNL) enriched at the mitral (OBmi) and glomerular (OBgl) layers. In the brainstem, molecular tissue regions enriched with distinct neuronal types were identified, such as INH_1-[Apt2b4_Nrgn_Zic1Grm5] in the pallidum (CNU_11-[PALv; PALm]), DEINH_1-[Pvalb_Hs3st4_Ramp3] in the TH_1-[RT], and DEGLU 3-[Necab1_C1ql3] in the dorsal-medial thalamus TH_3-[THm].

Although many glial cell types did not show strong tissue region-specific distribution (FIG. 53B), a few exceptions were observed. First, the results confirmed previous reports of region-specific astrocyte subtypes, including in the telencephalon (AC 2,3), non-telencephalon (AC_1), cerebellar Purkinje cell layer (AC 4), fiber tracts (AC_5), and meninges (AC_6) (FIGS. 53B and 58A). Second, the region-specific distribution of the oligodendrocyte lineage was examined, including oligodendrocyte precursor cells (OPC) and oligodendrocytes (OLG_1-3). Results showed that (i) in the cerebral cortex, OPC-OLG cells in deeper layers tended to be more mature, and (ii) the hindbrain contained a higher percentage of OLG at more mature stages than the forebrain and midbrain (FIGS. 63F-63J), which aligned with a recent report on the human OLGs that the ratio of oligodendrocytes to OPCs was higher in the brainstem than other regions.

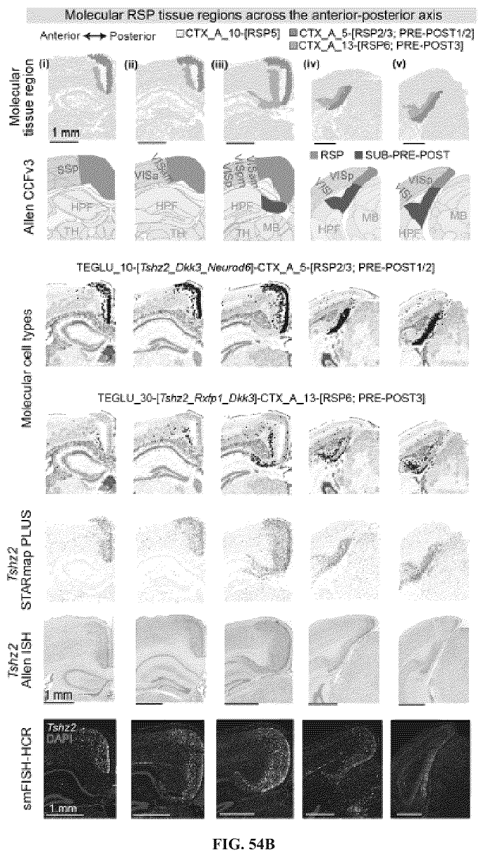

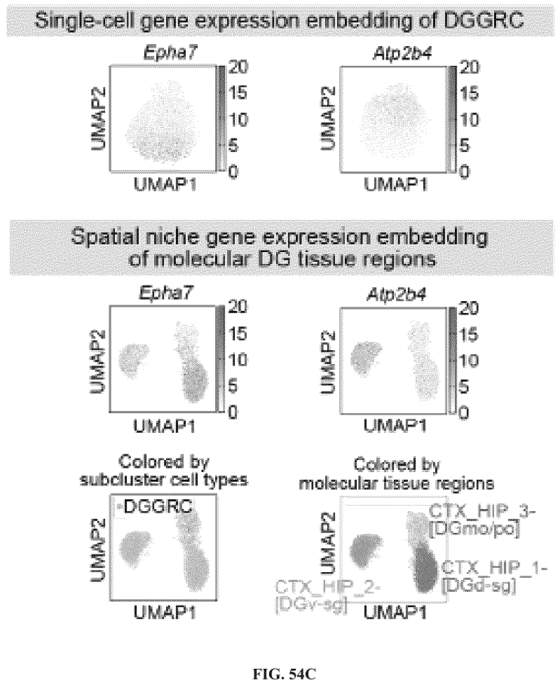

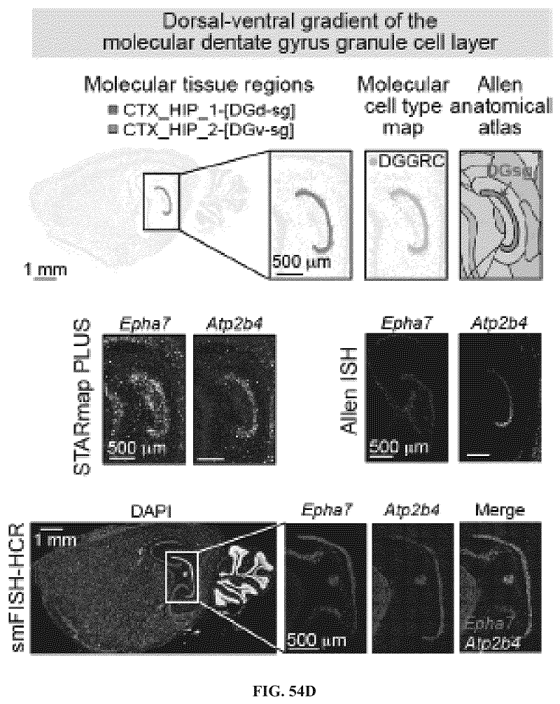

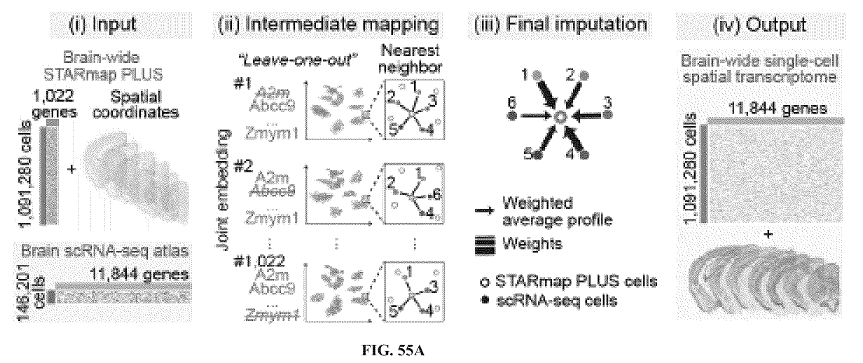

New tissue structures that differ from current Common Coordinate Framework (CCF) brain anatomy, along with associated cell types and gene markers were discovered. First, molecular tissue regions illustrated spatial gene expression patterns that were not captured by anatomical structures, such as a fine lamina (CTX_A_3-[L2/3]) in the superficial layer of anatomical cerebral cortical L2/3 (FIG. 54A) marked by high expression of Wfs1 and enriched with molecular cell types TEGLU_16-[Matn2_Cpne6_Lypd1] and TEGLU_19-[Cux2_Nptx2_C1ql3]. In contrast, the canonical L2/3 marker Cux2 occupied both molecular tissue regions CTX_A_3-[L2/3] and CTX_A_4-[L2/3]. The gene expression patterns of Wfs1 and Cux2 were also observed in the Allen ISH database and validated by smFISH-HCR™ (FIG. 54A).

Second, the molecular tissue region maps brought new information to refine the anatomical (Common Coordinate Framework) CCF. For example, three molecular tissue regions corresponding to the retrosplenial cortex (RSP) were identified, including CTX_A_5, CTX_A_10, and CTX_A_13. All three regions had clear marker genes and unique cell type compositions: Tshz2 as the pan-marker for CTX_A_5,10,13; TEGLU_10-[Tshz2 Dkk3 Neurod6] in CTX_A_5, TEGLU_35-[Tshz2_Cbln1_Nrep] in CTX_A_10, and TEGLU_30-[Tshz2_Rxfp1_Dkk3] in CTX_A_13 (FIG. 54B). While these molecular tissue regions aligned with the anatomical RSP towards the anterior of the anterior-posterior (A-P) axis (FIG. 54B, i and ii), posteriorly, they had less consensus with anatomical CCF and may potentially provide refinements to it. Specifically, posterior CTX_A_5 and 13 occupied the anatomical SUB-PRE-POST (subiculum-presubiculum-postsubiculum) region (FIG. 54B, iv and v). Furthermore, the regions defined as anatomical posterior RSP in CCF shared the same molecular tissue region composition with the adjacent anatomical visual cortex (FIG. 54B, iv and v). Between the anterior and posterior parts, CTX_A_5 and 13 occupied both anatomical RSP and the anatomical SUB-PRE-POST regions (FIG. 54B, iii). Given the discrepancy between the results and the current CCF anatomical labels, the molecular tissue region maps were confirmed by further revealing the A-P distribution of the molecular tissue region marker gene Tshz2, both in the Allen ISH database and by smFISH-HCR™ validation (FIG. 54B). The result may provide insight into a recent related study, which identified that the anatomically defined anterior and posterior RSP showed different functions in memory formation in rodents. Specifically, the inhibition of the anatomical posterior RSP selectively impaired the visual contextual memory information, suggesting that anatomical posterior RSP defined in CCF may contain part of the adjacent visual cortex. Notably, the anatomical RSP was traditionally defined by cell and tissue morphology (i.e., Nissl staining or neurofilament staining) without gene expression information. Hence, the molecular tissue regions (marked by Tshz2, Cxcl14, and Rxfp1, FIGS. 54B and 63K) may be more accurate in delineating RSP and its subregions.

Third, cases were observed wherein the joint single-cell and spatial definition of cell types resolved cell heterogeneity better than single-cell gene expression alone. While the dentate gyrus granule cells (DGGRC) largely formed a homogeneous cluster in the single-cell gene expression latent space, they fell into two distinct molecular tissue region clusters (CTX_HIP_1-[DGd-sg] and CTX_HIP_2-[DGv-sg]) in the spatial niche gene expression latent space, marked by enriched expression of Epha7 and Atp2b4, respectively (FIG. 54C). Allen ISH database and smFISH-HCR™ validation confirmed the marker gene gradients along the dorsal-ventral (D-V) axis (FIG. 54D). This unique molecular tissue region segmentation through spatial niche gene expression may provide insights into functional transitions along the D-V axis of the hippocampus.

### Example 8.4: Transcriptome-Wide Gene Imputation

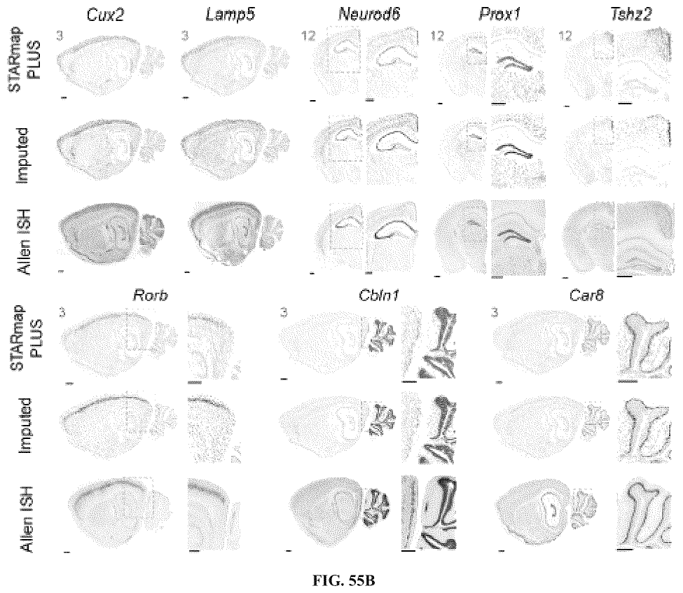

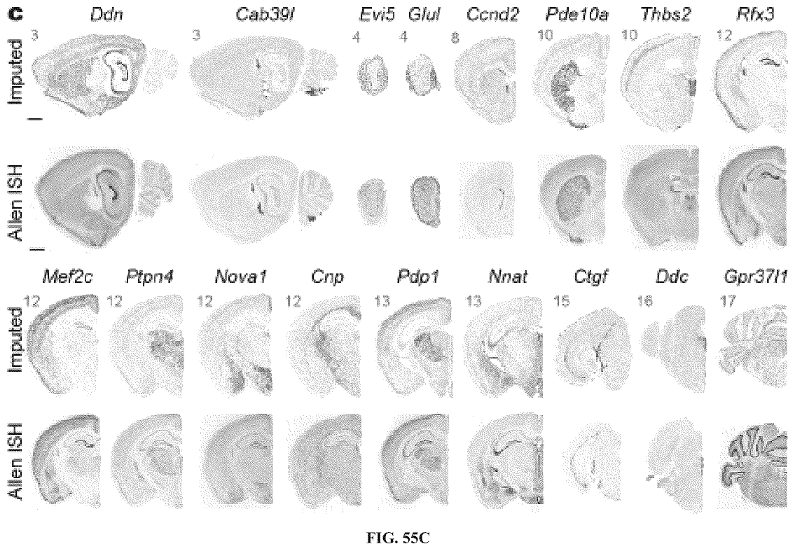

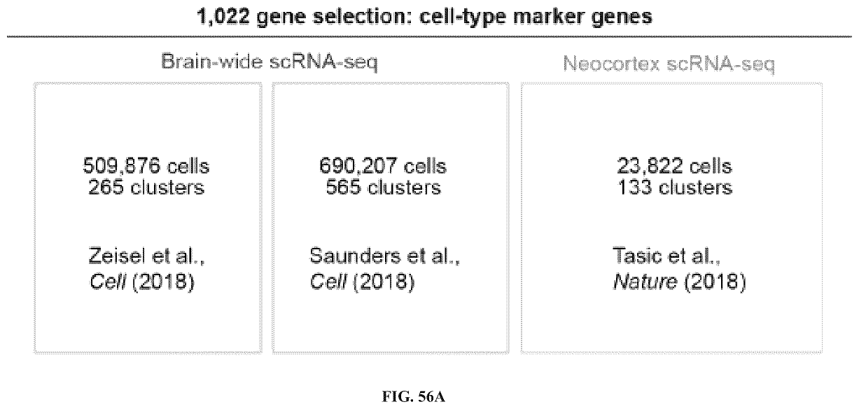

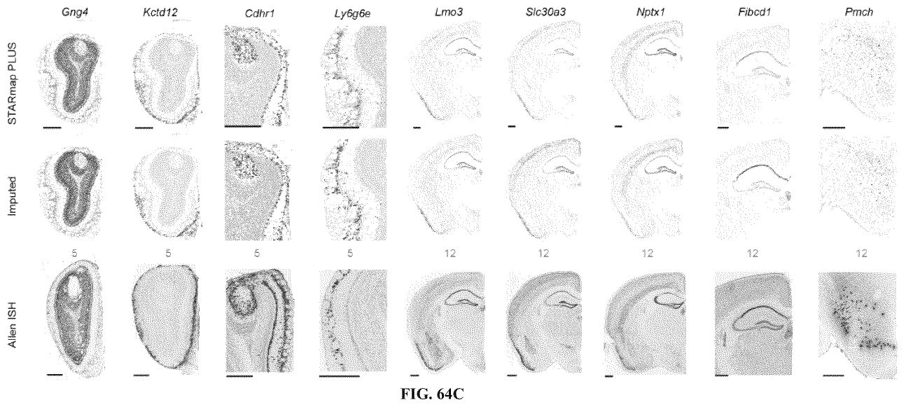

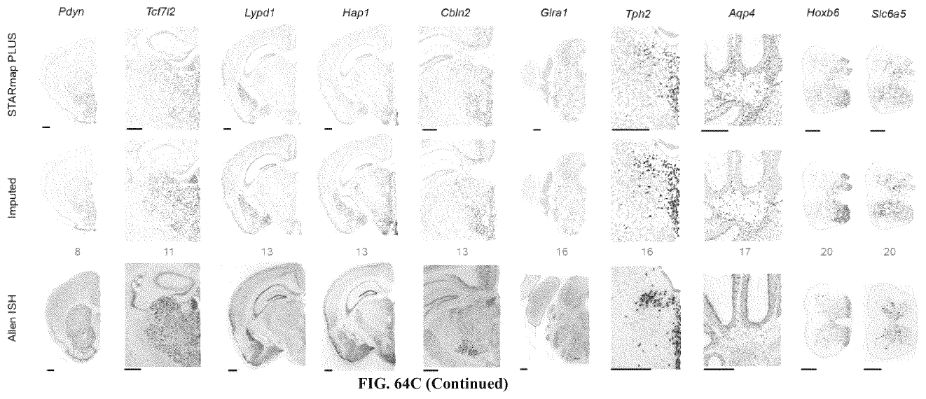

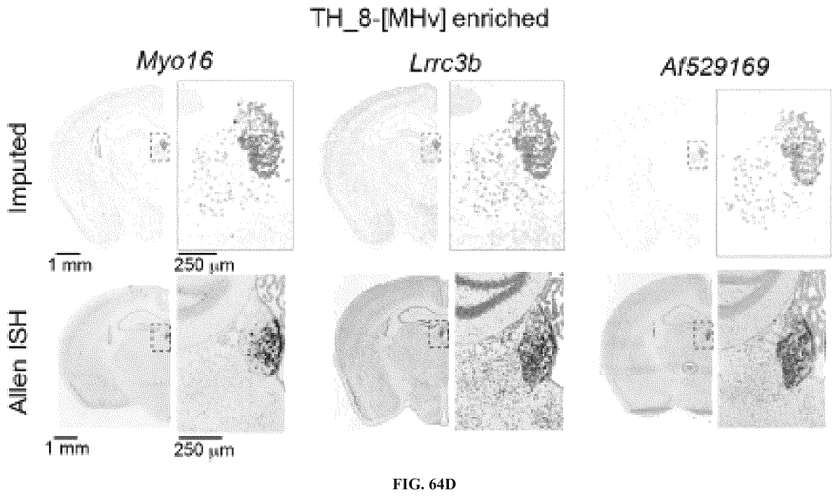

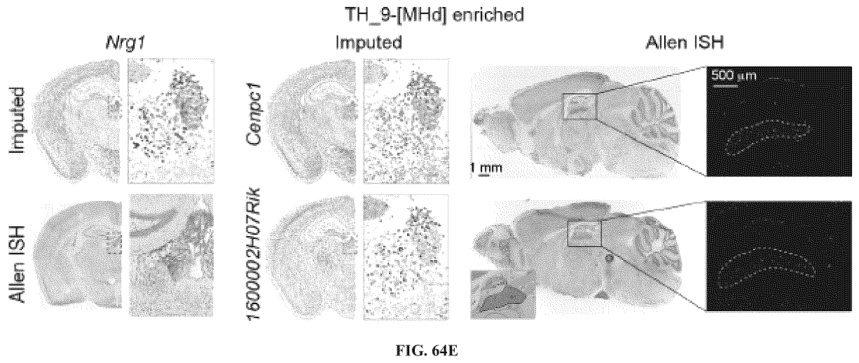

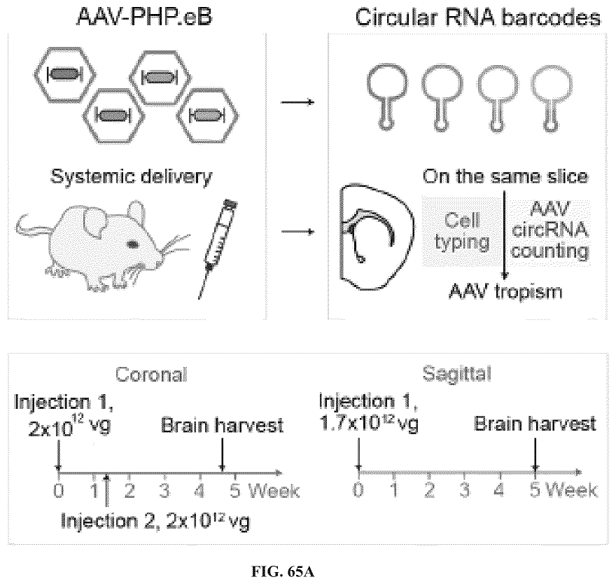

To establish transcriptome-wide spatial profiling of the mouse CNS, single-cell transcriptomic profiles were imputed using a previously reported mutual nearest neighbors (MNN) imputation method (Lohoff, T. et al. Nat. Biotechnol. 40, 74-85 (2022)). Specifically, using 1,022-gene STARmap PLUS measurements and a scRNA-seq atlas as inputs, intermediate mappings were generated using a leave-one-(gene)-out strategy to determine optimal nearest neighbor size (FIG. 64A) and compute weights between STARmap PLUS cells and scRNA-seq cells for the final imputation. As a result, 11,844-gene expression profiles were imputed for 1.09 million cells in the STARmap PLUS datasets, creating a transcriptome-wide spatial cell atlas of the mouse CNS (FIG. 55A).

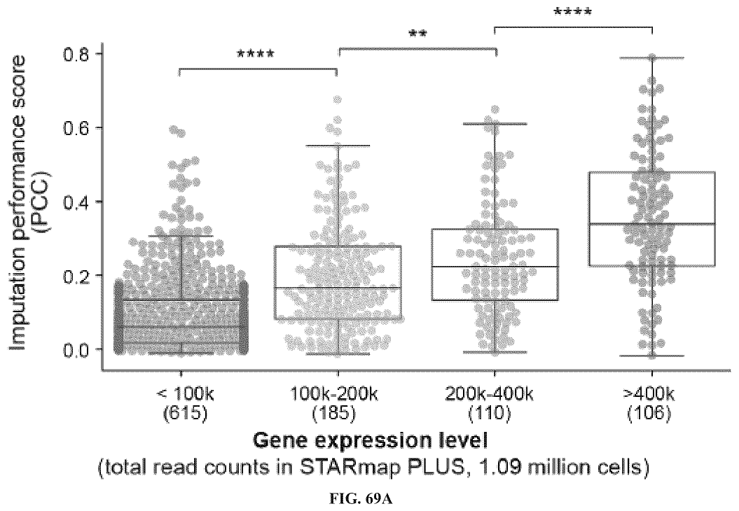

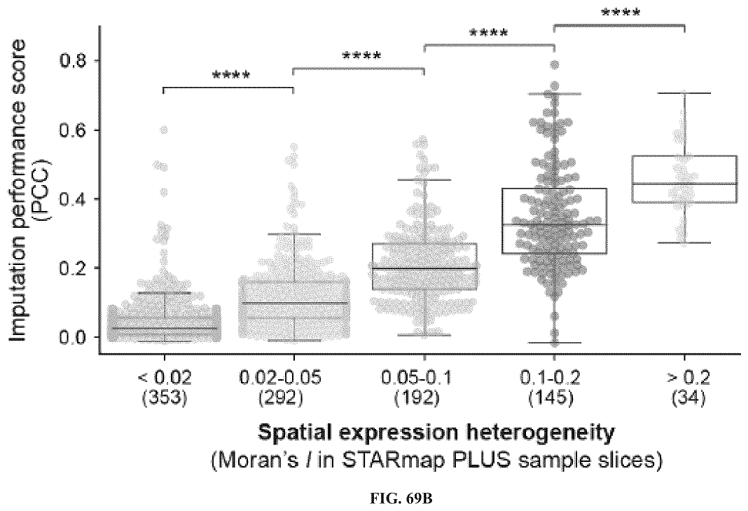

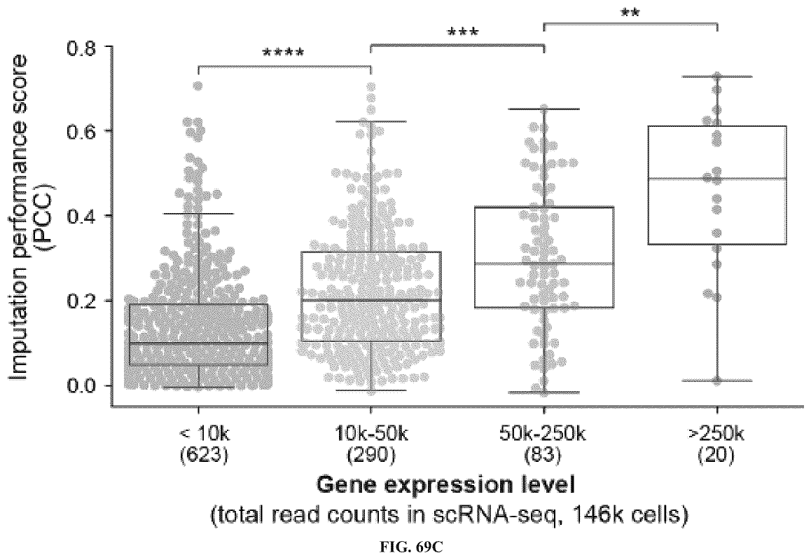

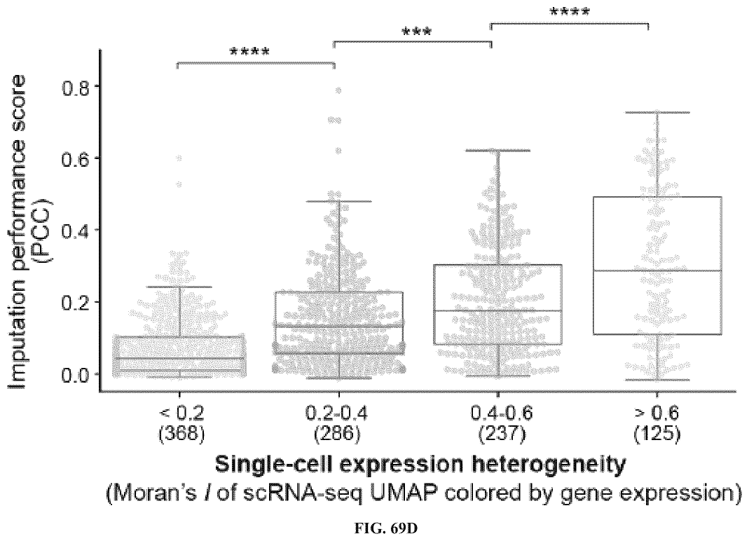

To validate the final imputation results, they were compared with ground-truth measurements from the STARmap PLUS and the Allen ISH database. In general, higher imputation performance was observed for genes with higher spatial and single-cell expression heterogeneity (FIGS. 64B and 69A-69D). For example, regional markers showed consistent spatial patterns across imputed and experimental results: Cux2 in cortical layers 2-4, Rorb in the cortical layer 4, Prox1 in the DG, Tshz2 in the RSP, Lmo3 in the piriform (PIR), Pdyn in the ventral striatum, Gng4 in the olfactory bulb granular layer, and Hoxb6 and Slc6a5 in the spinal cord (FIGS. 55B and 64C). Additionally, cell-type markers for both abundant and rare cell types were accurately imputed: cortical interneuron marker Lamp5, cerebellum neuron marker Cbln1, Purkinje cell marker Car8, and serotonergic neuron marker Tph2 (FIGS. 55B and 64C).

The imputed results of unmeasured genes were further benchmarked with the Allen ISH database. The imputed results successfully predicted the spatial patterns of unmeasured genes (FIG. 55C), especially cell-type marker genes, such as Cab39l (choroid epithelial cells, CHOR), Cnp (oligodendrocytes), and Ddc (dopaminergic neurons). The imputed results could also predict the relative regional expression of genes that express across multiple regions, such as Rfx3 (a transcription factor highly expressed in DG, PIR, and choroid plexus, and modestly in cortical L2/3, DG, and ependyma), Nova1 (an RNA-binding protein densely expressed in RSP L2/3, amygdala, and medial hypothalamic nuclei, and sparsely in the LHb), and Nnat (a proteolipid highly expressed in the ependyma, and modestly in the CA3, amygdala, and medial brainstem).

Finally, it was asked whether it was possible to uncover more tissue region-specific marker genes from the imputed results. Taking the ventral medial habenula (TH_8-[MHv]) as an example, in addition to its markers in the 1,022-gene list (e.g., Lrrc55, Gm5741, Nwd2, and Gng8), 108 genes from the imputed gene list were identified that were enriched in TH_8-[MHv](z-score>5), including Af529169, Lrrc3b, and Myo16, cross-validated with the Allen ISH database (FIG. 64D). For the dorsal medial habenula (TH_9-[MHd]), in addition to Wif1, Kcng4, and Pde11a, Nrg1, Cenpc1, and 1600002H07Rik were identified as enriched genes (FIG. 64E).

Collectively, by combining the molecular-resolution, brain-wide, large-scale STARmap PLUS datasets with a scRNA-seq atlas, a transcriptome-wide spatial cell atlas of the mouse CNS was generated with single-cell resolution. This imputed, expanded atlas can be a valuable resource to discover spatially variable genes, spatially co-regulated gene programs, and cell-cell interactions.

### Example 8.5: Quantitative AAV-PHP.eB Tropism Charts

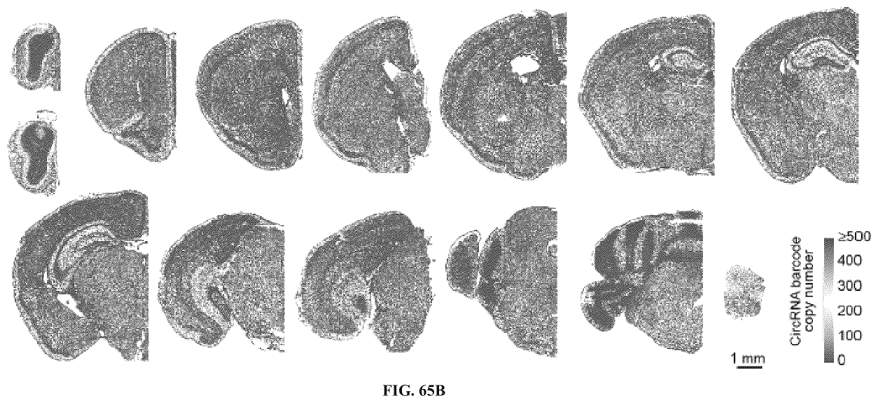

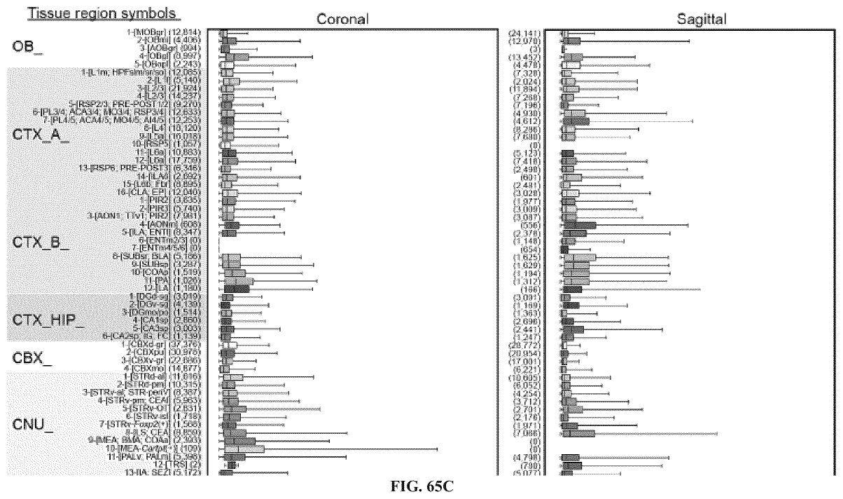

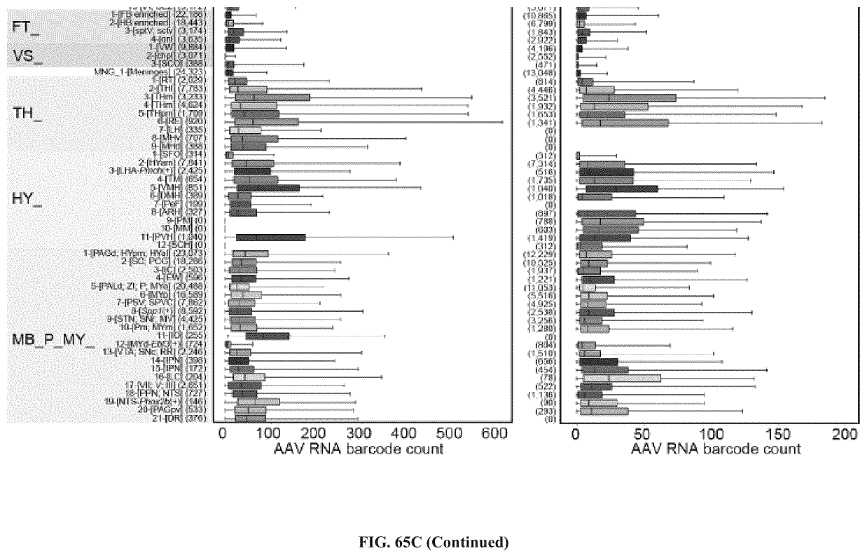

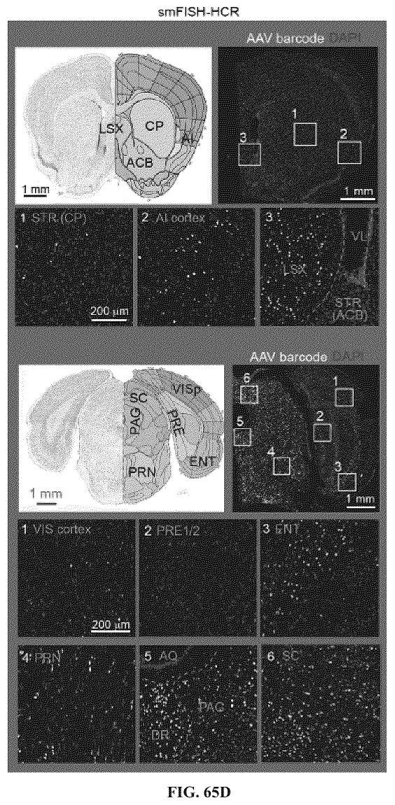

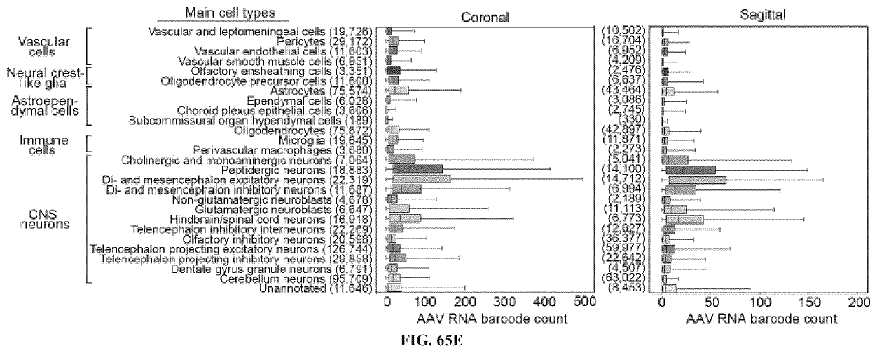

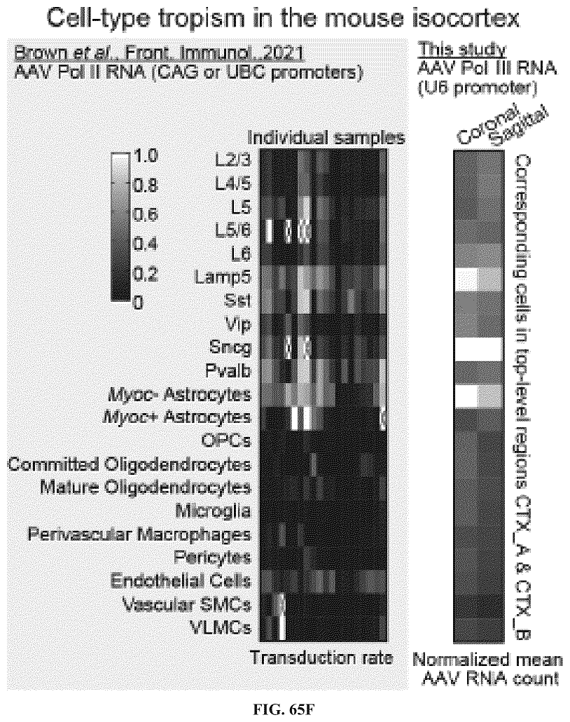

Experiments were undertaken to characterize the cell-type and tissue-region tropisms of AAV, the leading in vivo transgene delivery tool in neuroscience research. One AVV variant, PHP.eB, can efficiently cross the blood-brain barrier, allowing for brain-wide gene expression. To profile PHP.eB tropism in single cells, RNA barcoding and STARmap PLUS detection was combined, quantifying copy numbers of AAV RNA barcodes and endogenous genes in individual cells (FIGS. 12A, 12B, and 65A). For optimal expression across cell types, a highly expressed and stable circular RNA (Litke, J. L. et al. Nat. Biotechnol. 37, 667-675 (2019)) was designed under a generic Pol III-transcribed U6 promoter (FIG. 56C) rather than Pol II promoters with potential cell-type bias. A good correlation was observed between the coronal and sagittal replicates (Pearson's r≥0.837, P<0.0001), supporting the potency and robustness of the experimental and computational approaches presented herein for cell-type tropism profiling.

Then, AAV-PHP.eB tropism was assessed across molecular tissue regions. Among all brain regions, higher RNA barcode expression in the brainstem compared to the cerebrum (FIGS. 12C and 65B) and higher expression in neuron-rich regions than glia-rich regions (e.g., fiber tracts, ventricles, meninges, the choroid plexus, and the subcommissural organ;. FIGS. 12E and 65C) was observed, in general. Among neuron-rich regions, thalamic molecular tissue regions showed the highest transduction (FIGS. 12C, 12E, 65B, and 65C). Then, using smFISH-HCR™, the regional preferences of PHP.eB U6 transcripts was validated, for example, for the brainstem over the cerebrum and for the lateral septal complex (LSX) over the rest of the striatum (FIG. 65D).

Next, AAV-PHP.eB tropisms were examined across molecular cell types. The following were recapitulated: (i) the known tropism of PHP.eB towards neurons and astrocytes (FIGS. 12E and 65E-65F) and (ii) the preference of PHP.eB for Myoc⁻ astrocytes (AC_1˜5) over Myoc⁺ astrocytes (AC_6) (P<0.001, t-test). In other glial cells, OLG, OPC, OEC, vascular cells, and immune cells showed modest PHP.eB transduction. Epithelial cells were the lowest among all cell types in RNA barcode expression, including EPEN, CHOR, and subcommissural organ hypendymal cells (HYPEN) (FIGS. 12E and 65E). The PHP.eB transduction profile marked by viral Pol III RNA largely aligned with a previous report using viral Pol II mRNA in the isocortex (FIG. 65F). PHP.eB tropism profiles were further characterized among subcluster cell types. In summary, the mouse molecular CNS atlas offered valuable opportunities for in situ deep characterizations of viral tool tropisms.

### Example 8.6: Imputation Performance and Evaluation: Gene Expression Features Associated with Imputation Performance

Using the genes with STARmap PLUS measured ground-truth, the following four gene expression features were examined for their association with the imputation performance score in the “leave-one-out” intermediate imputation (FIGS. 69A-69D).
(1) Gene expression level in STARmap PLUS. Genes were categorized into four groups based on total read count in the STARmap PLUS dataset. Imputation performance shows an increasing trend as gene expression level increases (FIG. 69A; Pearson r=0.443, P=4.6e-50).
(2) Spatial expression heterogeneity in STARmap PLUS. For each gene, Moran's I (a coefficient measuring overall spatial autocorrelation) for the gene's spatial expression was calculated for each of the 20 sample slices and then averaged, to represent the degree of patterned spatial expression. A higher Moran's I represented more patterned spatial gene expression. A positive correlation was observed between the spatial pattern and imputation performance (FIG. 69B, Pearson r=0.738, P=2.3e-175).
(3) Gene expression in scRNA-seq dataset. Similar to (1), higher imputation performance was observed for genes with higher read counts in the scRNA-seq dataset (FIG. 69C, Pearson r=0.209, P=1.7e-11).
(4) Single-cell expression heterogeneity in scRNA-seq dataset. The degree of cell expression specificity of a gene was quantified by calculating Moran's I of the scRNA-seq UMAP plot colored by the gene's expression. Genes with a higher Moran's I on UMAP (usually cell cluster marker genes) tended to have better imputation performance (FIG. 69D, Pearson r=0.517, P=1.3e-70).

Gene expression heterogeneity in space and in single cells had a greater impact on imputation performances compared to gene expression levels (FIGS. 69A-69D), and genes with expression heterogeneity tend to have better imputation performance (FIG. 64B). These observations were consistent with a recent spatial expression gene imputation report, which showed that cell type-specific expressed genes and more highly expressed genes exhibit higher prediction accuracy. A gene's cell-type specificity (e.g., examining single-cell expression profiles in an atlas), spatial distribution (e.g., referencing Allen In Situ Hybridization database), and expression level can be important considerations when evaluating and judging gene imputation results.

The above Examples present a comprehensive spatial molecular atlas across the entire mouse CNS at 200 nm resolution, encompassing over one million cells with 1,022 genes measured by STARmap PLUS. The following were clustered and annotated providing a roadmap for investigating CNS-wide gene-expression patterns and cell-type diagrams in the context of brain anatomy: 26 main molecular cell types, 230 subtypes, 106 molecular tissue regions, and ˜2,000 molecular spatial cell types jointly defined by single-cell and niche gene expression profiles in 3D space (FIGS. 51A-53B). This unbiased molecular survey of the brain allowed for the discovery of new molecular cell types and tissue architectures (FIGS. 54A-54D). The 1,022 gene panel was expanded to the transcriptome scale by scRNA-seq atlas data integration and gene imputation (FIGS. 55A-55C).

The strategy and the resulting datasets had the following advantages. First, measuring RNA molecules in situ minimized the disturbance from sample preparation on single-cell expression profiles. Second, among spatial transcriptome mapping methods, STARmap PLUS is unique in its high spatial resolution (200-300 nm) in all three dimensions, enabling faithful capture of 3D tissue structures with molecular gene expression information. In the future, this molecular resolution mapping of cell transcripts and nuclear staining (FIG. 51F) may enable multimodal data analysis, such as joint cell typing by combining cell morphology and spatial transcriptomics. Third, the molecular spatial profiling demonstrated herein further enabled molecular tissue segmentation and data integration across different samples and technology platforms, leading to a more accurate and reproducible unified molecular definition of tissue regions compared to human-annotated anatomy. Finally, multiplexing measurements in the same sample allowed experimental integration of endogenous cellular features with exogenously introduced genetic labeling or perturbation, as illustrated by the AAV-PHP.eB tropism profiling in the mouse CNS (FIGS. 65A-65F). This systematic strategy can be adapted to simultaneously profile tropisms of multiple AAV capsid variants or screen various cell-type-specific promoter and enhancer sequences within the same sample by barcoding each variant, enabling cell-type resolved, tissue-level characterization of therapeutics engagement and responses.

In conclusion, herein are provided an organ-wide, single-cell, and spatially resolved transcriptome profiles of the mouse CNS at molecular resolution. These datasets offer potential for integration with other modalities, such as chromatin measurements, cell morphology, and cell-cell communication. This scalable experimental and computational framework may be applied to map whole-organ and whole-animal cell atlases across species and disease models, facilitating the study of development, evolution, and disorders. The atlas was complemented with an online database, mCNS_atlas, with exploratory interfaces (Error!Hyperlink reference not valid.brain.spatial-atlas.net), serving as an open resource for neurobiological studies across molecular, cellular, and tissue levels.

The results described herein above, were obtained using the following methods and materials.

### Plasmids

Sequences encoding the circular RNA downstream of a U6+27 promoter (U6+27-pre-racRNA) were adopted from the Tornado system (Addgene plasmid #124362; Litke, J. L. et al. Nat. Biotechnol. 37, 667-675 (2019)) and synthesized by GenScript. Specifically, the pre-racRNA was designed to contain a unique 25-nucleotide (nt) barcode region and a shared 25-nt common sequence to enable STARmap PLUS detection (FIG. 56C-56D). The U6+27-pre-racRNA sequence was inserted into the vector pAAV-hSyn-mCherry (Addgene plasmid #114472) between MluI and XbaI sites, resulting in plasmid pAAV-U6-racRNA. AAV packaging plasmids (kiCAP-AAV-PHP.eB and pHelper) were used.

### Virus Production and Purification

AAV-PHP.eB expressing circular RNA barcodes were produced and purified as described in Chan, K. Y. et al. Nat. Neurosci. 20, 1172-1179 (2017); Goertsen, D. et al. Nat. Neurosci. 25, 106-115 (2022). Briefly, pAAV-U6-racRNA and AAV packaging plasmids (kiCAP-AAV-PHP.eB and pHelper) were co-transfected into HEK 293T cells (ATCC® CRL-3216™) using polyethylenimine at the ratio of 1:4:2 based on micrograms (ug) of DNA with 40 ug in total per 150-mm dish. 72 hours after transfection, viral particles were harvested from the medium and cells. The mixture of cells and medium was centrifuged to form cell pellets. The cell pellets were suspended in 500 mM NaCl, 40 mM Tris, 2.5 mM MgCl₂, pH 8, and 100 U/mL of salt-activated nuclease (SAN, Arcticzymes) at 37° C. for 1 hour. Viral particles from the supernatant were precipitated with 40% polyethylene glycol (Sigma, 89510-1KG-F) dissolved in 500 mL 2.5 M NaCl solution and combined with cell pellets for further incubation at 37° C. for another 30 min. Afterwards, the cell lysates were centrifuged at 2,000 g, and the supernatant was loaded over iodixanol (Optiprep, Sigma; D1556) step gradients (15%, 25%, 40%, and 60%). Viruses were extracted from the 40/60% interface and the 40% layer of iodixanol gradients. Then viruses were filtered using Amicon filters (EMD, UFC910024) and formulated in sterile phosphate-buffered saline (PBS). Virus titers were determined using qPCR to measure the number of viral genomes (vg) after DNase I treatment to remove the DNA not packaged and then proteinase K treatment to digest the viral capsid and expose the viral genome. Quantified linearized plasmids of pAAV-U6-racRNA were used as a DNA standard to transform the Ct value to the amount of viral genome. The virus titer of AAV-PHP.eB.1 (barcode set 1) for coronal samples: 2×10¹³ vg/mL; AAV-PHP.eB.2 (barcode set 2) for sagittal samples: 1.7×10¹³ vg/mL.

### Mice and Tissue Preparation

The following animals were used in this study: C57BL/6 (strain code: 475, female, 8-10 weeks old) and B6.Cg-Tg(Thy1-YFP)HJrs/J (003782, male, 5 weeks old) purchased from the Charles River Laboratories and Jackson Laboratory (JAX), respectively. Animals were housed 2-5 per cage and kept on a reversed 12-hour light-dark cycle with ad libitum food and water at the temperature of 65-75° F. (˜18-23° C.) with 40-60% humidity. For virus injection, mice were anesthetized with isoflurane (3-5% induction, 1-2% maintaining). Mouse CNS tissues were sampled at least four weeks post-injection, when viral responses were shown to return to the control level to minimize the side effect of AAV infection on cell typing.

### Mouse Brain Coronal Sections and Spinal Cord Transverse Sections:

Intravenous administration of AAV-PHP.eB.1 at 2×10¹² vg was performed by injection into the retro-orbital sinus of adult mice (C57BL/6, female, 8-10 weeks of age). One week after the first injection, a second injection was administered to enhance expression. Thirty days after the first injection, mice were anesthetized with isoflurane (FIG. 65A). The brain tissue was collected after rapid decapitation. The spinal cord was isolated using hydraulic extrusion to reduce handling time and the risk of damage to the tissue. Briefly, the large end of a 200 pL non-filter pipette tip was trimmed and fit firmly onto a 5 mL syringe. Next, the spinal column was cut on both sides past the pelvic bone through the rostral-caudal axis, straightening and trimming at both proximal- and distal-most ends until the spinal cord was visible. A 5 mL syringe filled with ice-cold PBS (Gibco, 10010049) was inserted at the distal-most end of the spinal column, and steady pressure was applied to extrude the spinal cord into a 10 mm Petri dish filled with sterile PBS on ice. The lumbar segments of the spinal cord tissue were collected. Tissues were placed in O.C.T. (Fisher, 23-730-571), frozen in liquid nitrogen, and sliced into 20 μm sections using a cryostat (Leica CM1950) at −20° C.

### Mouse Brain Sagittal Sections:

Intravenous administration of AAV-PHP.eB.2 at 1.7×10¹² vg was performed by injection into the retro-orbital sinus of an adult Thy1-EYFP mouse (B6.Cg-Tg(Thy1-YFP)HJrs/J, male, five weeks of age). After five weeks of expression, mice were anesthetized with isoflurane and transcardially perfused with 50 mL ice-cold DPBS (Dulbecco's Phosphate Buffered Saline, Sigma-Aldrich, D8537) (FIG. 65A). The brain tissue was then removed, split into two hemispheres, placed in O.C.T., frozen in liquid nitrogen, and sliced into 20 μm sagittal sections using a cryostat (Leica CM1950) at −20° C.

### 1,022-Gene List Selection and STARmap PLUS Probe Design

Cell-type marker genes and most differentially expressed genes were extracted from single-cell RNA-sequencing studies that systematically surveyed the adult mouse central nervous system, which included multiple brain regions from the forebrain to the hindbrain and sampled the cells with minimum selection. The list was further supplemented with the Allen Mouse Brain transcriptome database markers. The list was curated to 1,022 genes to be uniquely encoded by 5-digit identifiers (FIG. 56A).

STARmap PLUS probes for the 1,022 genes were designed as described in Wang, X. et al. Science 361, eaat 5691 (2018) and Zeng, H. et al. Nat. Neurosci. (2023) doi:10.1038/s41593-022-01251-x with modifications to further improve the specificity of target transcript detection. The backbone of padlock probes contains a 5-nt gene-specific identifier and a universal region where reading probes align (FIG. 56B). In addition, a second 3-nt barcode was introduced to the DNA-DNA hybridization region between a pair of primer and padlock probes to reduce the possibility of false positives caused by intermolecular proximity where the primer for transcript identity A leads to circularization of the padlock hybridized to transcript identity B. For the SEDAL seq step, the homemade sequencing reagents included six reading probes (RI to R6) and 16 two-base encoding fluorescent probes (2base_F1 to 2base_F16) labeled with Alexa 488, 546, 594, and 647.

To detect RNA barcodes, a primer was designed to hybridize to the common 25-nt region while a pool of padlock probes was designed to hybridize to variable 25-nt barcode region, converting the barcode into a barcode-unique identifier (FIG. 56D). This identifier was sequenced in one round of SEDAL seq by an orthogonal reading probe (R7 for coronal samples and R8 for sagittal samples) and four one-base encoding fluorescent probes (1base_F1 to 1base_F4) labeled with Alexa 488, 546, 594, and 647.

### STARmap PLUS

The STARmap PLUS procedure was performed as described in Wang, X. et al. Science 361, eaat 5691 (2018) and Zeng, H. et al. Nat. Neurosci. (2023) doi:10.1038/s41593-022-01251-x with minor modifications.

### Sample Preparation:

Glass-bottom 6- or 12-well plates (MatTek, P06G-1.5-20-F and P12G-1.5-14-F) were treated with methacryloxypropyltrimethoxysilane (Bind-Silane, GE Healthcare, 17-1330-01), followed by a poly-D-lysine solution (Sigma-Aldrich, A-003-E). #2 Micro cover glasses (12 mm or 18 mm, Electron Microscopy Sciences, 7′22260 or 72256-03) were pretreated with Gel Slick solution (Lonza, 50640) following the manufacturer's instructions for later polymerization. 20 μm coronal and sagittal slices were mounted in the pretreated glass-bottom 12-well and 6-well plates, respectively. Tissue slices were fixed with 4% PFA (Electron Microscopy Sciences, 15710-S) in PBS at room temperature for 10 min, permeabilized with pre-chilled methanol (Sigma-Aldrich, 34860-1L-R) at −80° C. for 30 min, and re-hydrated with PBSTR/Glycine/YtRNA (PBS with 0.1% Tween-20 [TEKNOVA INC, 100216-360], 0.1 U/μL SUPERase-In [Invitrogen, AM2696], 100 mM Glycine, 1% Yeast tRNA [Invitrogen, AM7119]) at room temperature for 15 min before hybridization. For sagittal slices, the step of methanol treatment was skipped, and the sample was permeabilized with 1% Triton X-100 (Sigma-Aldrich, 93443) in PBS with 0.1 U/pL SUPERaseIn, 100 mM Glycine (VWR, M103-1KG), and 1% Yeast tRNA at room temperature for 15 min.

### Library Construction:

The reaction volumes listed below were for 12-well plate wells. For 6-well plate wells, the reaction volume was doubled. Stock SNAIL probes were dissolved to 50 nM or 100 nM per probe in IDTE pH 7.5 buffer (IDT, 11-01-02-02). The final concentration per probe for hybridization was as follows: SNAIL probes for mouse 1,022-gene, 5 nM; primers for RNA barcodes, 100 nM; padlock probes for RNA barcodes, 10 nM for coronal samples, and 100 nM for sagittal samples. The brain slices were incubated in 300 μL hybridization buffer (2×SSC [Sigma-Aldrich, S6639], 10% formamide [Calbiochem, 344206], 1% Triton X-100, 20 mM RVC [Ribonucleoside vanadyl complex, New England Biolabs, S1402S], 0.1 mg/ml yeast tRNA, 0.1 U/μL SUPERaseIn, and SNAIL probes) at 40° C. for 24-36 hours with gentle shaking.

The samples were then washed at 37° C. for 20 min with 600 μL PBSTR (PBS, 0.1% Tween-20, 0.1 U/μL SUPERase-In) twice, followed by one wash at 37° C. for 20 min with 600 pL High Salt buffer (PBSTR, 4×SSC). After a brief rinse with PBSTR at room temperature, the samples were then incubated for two hours with a 300 μL T4 DNA ligase mixture (0.1 U/μL T4 DNA ligase [Thermo Scientific, EL0011], 1× T4 ligase buffer, 0.2 mg/mL BSA [New England Biolabs, B9000S], 0.2 U/μL of SUPERase-In) at room temperature with gentle shaking, followed by twice washes with 600 μL PBSTR. Then the sample was incubated with 300 μL rolling-circle amplification (RCA) mixture (0.2 U/μL Phi29 DNA polymerase [Thermo Scientific, EP0094], 1× Phi29 reaction buffer, 250 μM dNTP mixture [New England Biolabs, N0447S], 0.2 mg/mL BSA, 0.2 U/μL of SUPERase-In and 20 μM 5-(3-aminoallyl)-dUTP [Invitrogen, AM8439]) at 4° C. for 30 minutes for equilibrium and at 30° C. for two hours for amplification.

The samples were next washed twice in 600 μL PBST (PBS, 0.1% Tween-20) and treated with 400 μL 20 mM acrylic acid NHS ester (Sigma-Aldrich, 730300-1G) in 100 mM NaHCO₃ (pH 8.0) for one hour at room temperature. The samples were briefly washed with 600 μL PBST once, then incubated with 400 μL monomer buffer (4% acrylamide [Bio-Rad, 161-0140], 0.2% bis-acrylamide [Bio-Rad, 161-0142], 2×SSC) for 30 min at room temperature. The buffer was removed, and 25 μL of polymerization mixture (0.2% ammonium persulfate [Sigma-Aldrich, A3678], 0.2% tetramethylethylenediamine [Sigma-Aldrich, T9281] in monomer buffer) was added to the center of the sample, which was immediately covered by Gel Slick coated coverslip and incubated for one hour at room temperature under nitrogen gas atmosphere. The samples were then washed with 600 μL PBST twice for 5 min each. Except for sagittal brain slices, the tissue-gel hybrids were digested with Proteinase K (Invitrogen, 25530049, 0.2 mg/ml in 50 mM Tris-HCl 8.0, 100 mM NaCl, 1% SDS [Calbiochem, 7991]) at room temperature overnight, then washed with 600 μL 1 mM AEBSF (Sigma-Aldrich, 101500) in PBST once at room temperature for 5 min and another two washes with PBST. Samples were stored in PBST at 4° C. until imaging and sequencing.

### Imaging and Sequencing:

Before SEDAL seq, the samples were washed twice with the stripping buffer (60% formamide and 0.1% Triton X-100 in water) and treated with the dephosphorylation mixture (0.25 U/μL Antarctic Phosphatase [New England Biolabs, M0289L], 1× reaction buffer, 0.2 mg/mL BSA) at 37° C. for one hour. Each cycle of SEDAL seq began with two washes with the stripping buffer (10 min each) and three washes with PBST (5 min each). For the six-round of 1,022-gene SEDAL seq, the sample was then incubated with the “sequencing by ligation” mixture (0.2 U/μL T4 DNA ligase, 1× T4 DNA ligase buffer, 0.2 mg/mL BSA, 10 μM reading probe, and 300 nM of each of the 16 two-base encoding fluorescent probes) at room temperature for three hours. For the round of RNA barcode SEDAL seq, the sample was incubated with (0.1 U/μL T4 DNA ligase, 1λT4 DNA ligase buffer, 0.2 mg/mL BSA, 5 μM reading probe, 100 nM of each of the four one-base fluorescent oligos) at room temperature for one hour. After three washes with the wash and imaging buffer (10% formamide, 2×SSC in water, 10 min each) and DAPI staining (Invitrogen, D1306, 100 ng/mL), the sample was imaged in the wash and imaging buffer.

Images were acquired using Leica TCS SP8 or Stellaris 8 confocal microscopy using LAS X software (SP8: version 3.5.5.19976; Stellaris 8: version 4.4.0.24861) with a 405 nm diode, a white light laser, and 40× oil immersion objective (NA 1.3) with a voxel size of 194 nm×194 nm×345 nm. DAPI was imaged at the first round of 1,022-gene SEDAL seq and the round of RNA barcoding SEDAL seq to enable image registration (FIG. 52A).

### STARmap PLUS Data Processing

### Pre-Processing (Deconvolution, Registration, Spot-Calling)

Image deconvolution was achieved with Huygens Essential version 21.04 (Scientific Volume Imaging, The Netherlands, svi.nl), using the Classic Maximum Likelihood Estimation (CMLE) method, with SNR:10 and 10 iterations. Image registration, spot calling, and barcode filtering were applied according to previous reports (Wang, X. et al. Science 361, eaat 5691 (2018); Zeng, H. et al. Nat. Neurosci. (2023) doi:10.1038/s41593-022-01251-x).

### ClusterMap Cell Segmentation

The ClusterMap (He, Y. et al. Nat. Commun. 12, 5909 (2021)) method was used to segment cells by amplicons (mRNA spots) with quality control for gene spots with pre- and post-processing. First, a background identification process was used to filter input spots. Specifically, 10% of local low-density mRNA spots were considered as background noises and were removed before the downstream analysis. Second, an additional step of noise rejection was used after mRNA spot clustering as post-processing. Specifically, that did not overlap with DAPI signals were erased. These quality control steps for gene reads have been included in the analysis of all 20 coronal and sagittal datasets.

### Quality Control for Cells

First, low-quality cells were excluded with standard preprocessing procedures in Scanpy (Wolf, F. A., et al., Genome Biol. 19, 15 (2018)). Here 20 coronal and sagittal datasets were combined and analyzed together. The minimum gene number and cell number was set as 20, the minimum read count per cell as 30, and the maximum read count per cell as 1,300. After filtering, a data matrix of 1,099,408 cells by 1,022 genes was obtained. Then the matrix was normalized across each cell and logarithmically transformed. The effects of total read count per cell were regressed out and the data was finally scaled to unit variance.

### Batch Effect Evaluation and Correction

To evaluate batch effects, adjacent tissue slices were grouped into adjacent batches. Batch effect was checked across labeled batch samples A-J. The batch effect was first observed and corrected between coronal samples in groups C and D using Combat (Johnson, W. E., et al. Biostatistics 8, 118-127 (2007)). The batch effect between coronal and sagittal samples was also observed and corrected. The function scanpy.pp.combat was used for batch effect correction.

### Cell Type Annotations

Integration with scRNA-Seq Dataset

Harmony (Korsunsky, I. et al. Nat. Methods 16, 1289-1296 (2019)) was used to integrate STARmap PLUS datasets and a scRNA-seq dataset of the mouse nervous system. The overlapped 1,021 genes between the STARmap PLUS and the scRNA-seq experiments were used to compute adjusted principal components (PCs) and performed joint clustering to transfer main-level cell-type labels in the scRNA-seq dataset to STARmap PLUS identified cells. The function scanpy.external.pp.harmony integrate was used to perform the integration. The function scanpy.tl.leiden was used with a resolution equal to 1 to perform joint clustering.

### Main Cluster and Subcluster Cell-Type Annotation

The main-level clustering and annotation of STARmap PLUS identified cells were decided based on the integration of STARmap PLUS datasets with the public scRNA-seq dataset.

First, STARmap PLUS cells were integrated with cells in the scRNA-seq dataset. Second, joint Leiden clustering was performed on all integrated cells, recovering 53 joint clusters. Third, to transfer labels of cells in scRNA-seq datasets, the principle used is described as follows. Within each joint cluster, the cell type labels of scRNA-seq cells was checked. If the number of top-1 scRNA-seq cell-type labels within one joint cluster exceeded 80%, it indicated successful integration for multi-source single-cell datasets on this cell type. Therefore, this dominant top-1 scRNA-seq cell-type label was assigned to all STARmap PLUS cells in that joint cluster with high confidence. Otherwise, integration was regarded as unsuccessful and the joint cluster was temporarily labeled as ‘NA’. STARmap PLUS datasets were annoted at four levels using this principle using Rank 1 to Rank 4 cell-type labels in the scRNA-seq dataset. Specifically, cells were annoted into 4 cell types at Rank 1 level; 5 cell types at Rank 2 level, 13 cell types at Rank 3 level, and 22 cell types at Rank 4 level. There existed a portion of cells in NA types in levels of Rank 2 to Rank 4. A higher rank means more detailed annotations. Finally, the Rank 4 level annotation was defined as the main-level annotation (main cell types).

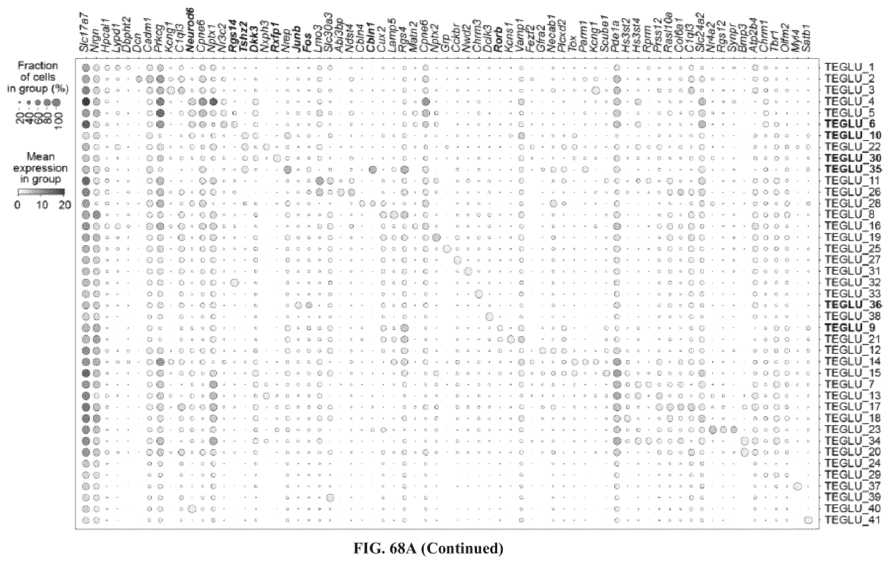

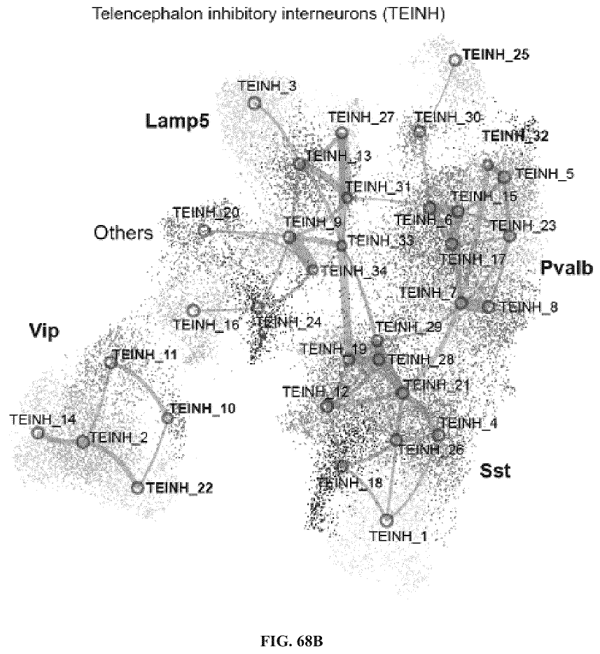

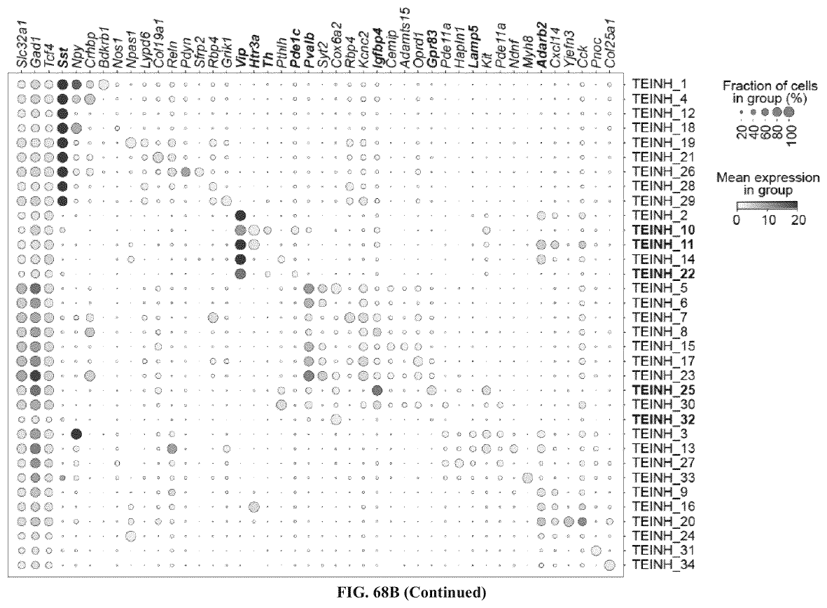

Individual cell types in the main-level annotation with the cells labeled as ‘NA’ were then investigated and detailed sublevel cell types were manually annotated (FIGS. 67A-68B). First, cells in each main-level cluster were extracted and Leiden clustering was performed to determine subclusters. Specifically, genes with a maximum read count per cell of less than 10 or genes that expressed over 5 counts were found in less than 10 cells, computed PCA and UMAP, were filtered out and Leiden clustering was performed on the UMAP space. Functions scanpy.tl.pca, scanpy.pp.neighbors, scanpy.tl.umap and scanpy.tl.leiden were used.

Second, each subcluster was annotated based on marker genes and spatial cell distribution. Specifically, the top five marker genes for each subcluster were first identified using scanpy.tl.rank_genes_groups. In each subcluster, the dot plot showing the fraction of cells expressing specific marker genes and the mean expression of specific marker genes were checked. The marker genes highly expressed across multiple cell types were recognized as common markers. The markers with specific expressions in a particular subcluster were identified as cluster-specific markers. In addition, those marker genes in other scRNA-seq databases were examined and confirmed. Then, the marker gene list was refined and the subclusters with the most relevant cell types were annoted based on the remaining marker genes. Second, to narrow down to a unique annotation or distinguish the subclusters with the same annotations, the spatial cell distribution of each subcluster was checked. It was observed that some subclusters were explicitly distributed in certain brain regions, such as peptidergic neurons in the hypothalamus and medium spiny neurons in the striatum, allowing us to rule out irrelevant candidates. As for the remaining undetermined subclusters based on marker genes and spatial distribution, they were with the most relevant annotated subclusters or split them further using Leiden clustering based on prior knowledge.

Third, cells were analyzed in the ‘NA’ cluster. These cells were assigned to valid cell types and combined into Rank 4 clusters when appropriate. Specifically, the following types were recovered from the Rank 4 ‘NA’ cells: subcommissural hypendymal cells (HYPEN); non-glutamatergic neuroblasts (NGNBL); Purkinje cells (CBPC, combined into Rank 4 cerebellum neurons); Th⁺ OBINH (OBINH_7, combined into Rank 4 olfactory inhibitory neurons). Additionally, vascular-like cells in the NA cluster were combined with Rank 4 vascular cells and re-clustered. Neuronal-like cells in the NA cluster were combined with Rank 4 di- and mesencephalon inhibitory neurons and Rank 4 hindbrain neurons and re-clustered (FIG. 67K). There remained 12 unannotated subclusters (1.8% of total cells) due to lack of annotatable marker genes (FIG. 67N), which may have resulted from the differences in sampling coverage between the scRNA-seq and STARmap PLUS datasets.

The cell-typing results in the Examples were based on the consensus between the STARmap PLUS dataset and the published scRNA-seq datasets, followed by manual annotation. The STARmap PLUS dataset mapped more cells than the previous scRNA-seq dataset, potentiating more detailed cell typing and annotations in the future.

A schematic summary of the cell typing workflow is shown in FIG. 57C.

### Near-Range Cell-Cell Adjacency Analysis

The number of edges between cells of each main cell type with cells of other main cell types was quantified as described in He, Y. et al. Nat. Commun. 12, 5909 (2021). Briefly, a mesh graph was constructed by Delaunay triangulation of cells in each sample using squidpy.gr.spatial neighbors. A ring of cells that were neighbors of the central cell in the mesh graph was considered to connect the central cell. Then a near-range cell-cell adjacency matrix was computed from spatial connectivity using squidpy.gr.interaction matrix. The matrix was normalized using row normalization followed by column normalization as shown in FIG. 59G.

### Molecular Tissue Region Analysis

### Molecular Tissue Region Clustering Based on Spatial Niche Gene Expression

For a given sample, the smoothed expression vector of each cell was represented by concatenating that of its k nearest spatial neighbors, including itself. The spatially smoothed-expression matrices for each sample were then stacked into a single dataset and passed into the principal component analysis (PCA) followed by Harmony (Korsunsky, I. et al. Nat. Methods 16, 1289-1296 (2019)) for integration. Clustering was then performed in principal component space using the Leiden algorithm followed by visualization using uniform manifold approximation and projection (UMAP) (McInnes, L., Preprint at arxiv.org/abs/1802.03426 (2018)).

The value k was set to 30 neighbors for the identification of broad anatomical regions (level 1), such as the neocortex. To identify subregions (level 2), such as individual neocortical layers, subclustering of each level 1 region was performed with varying k values depending on the morphology of expected subregions. For example, as meninges are inherently thin, subregions of meninges were also expected to be thin and thus require a smaller neighborhood size k in order to avoid smoothing away their finer structure. A final level of clustering was then applied to a subset of level 2 regions to identify more subregions (level 3) that were expected based on manual inspection of level 2 gene markers.

For a sample slice, when the number of cells in a cluster is smaller than the value k for smoothing, the concatenated spatial niche gene expression vector cannot be made. In this case, the cell was rejected from further subclustering. To take care of those rejected cells, post-processing was performed to transfer tissue region labels from their physical neighboring cells.

A resolution parameter must also be specified for each instance of clustering. Resolutions for each level of clustering were manually tuned to capture known anatomical features based on the Allen Institute Mouse Atlas as well as preliminary marker genes calculated using differentially expressed gene (DEG) analysis via the rank_genes_groups function in Scanpy (Wolf, F. A., et al., Genome Biol. 19, 15 (2018)).

To identify tissue region marker genes, the average expression of each gene across all the cells of each region was first calculated. Then for each gene, its percentage distribution across tissue regions was normalized to z-scores.

Finally, fragmented subclusters originating from different main clusters were manually combined when appropriate. To guide manual curation of spatial clustering, non-negative matrix factorization (NMF) (Lee, D. D. & Seung, H. S. Nature 401, 788-791 (1999)) was applied to the stacked and spatially smoothed expression matrix (i.e., the matrix passed into PCA/Harmony above), identifying anatomical factors along with corresponding gene factor loadings.

### Molecularly Tissue Region Label Post-Processing

Tissue region labels were first assigned for those cells missing annotation. First, under level-1 tissue region labels, the k-nearest-neighbors (kNNs, here k=5) smoothing was performed to assign a level-1 tissue region label for those cells missing level-1 annotation. Then, similarly, under level-2 and level-3 tissue region labels, respectively, the k-nearest-neighbors (kNNs, here k=5) smoothing was performed to assign a level-2 or level-3 tissue region label for those cells missing level-2 or level-3 annotation.

Smoothing was then performed based on level-3 tissue region labels (kNNs, here k=50), and some molecular tissue region labels were manually adjusted. First, cells in the “Meninges” molecular tissue regions were excluded from the smoothing process to minimize the effect on the nearby tissue regions. Second, it was observed that cell-sparse regions (e.g., molecular layers) would be overwhelmed by a nearby cell-dense region (e.g., granule cell regions) during this smoothing process. Therefore, the molecular tissue region cluster labels was manually kept unchanged for those cells (including OB_5-[OBopl] and CTX_HIP_3-[DGmo/po]).

### Allen Mouse Brain Common Coordinate Framework (CCFv3) Registration, Label Transfer, and Molecular Tissue Region Annotation

Registration of each STARmap PLUS tissue slice with Allen CCFv3 according to public resources was performed. Specifically, to match each STARmap PLUS slice to its corresponding CCF slice, images of STARmap PLUS cells colored by their identified cell types were first generated. Then one corresponding slice image was manually extracted from Allen CCFv3 slides. Next, paired points in the STARmap PLUS slice and the corresponding Allen CCFv3 slice were manually clicked for registration. The package AP_histology (Peters, A. AP_histology. GitHub repository, github.com/petersaj/AP_histology (2019)) provided the analysis.

After registration, a paired Allen CCFv3 slice was in-hand for each of the STARmap PLUS tissue slices. An inverse transformation was applied to the paired Allen CCFv3 slices and labels of Allen CCF anatomical regions were assigned to cells in STARmap PLUS tissue slices to facilitate molecular tissue region annotation.

### RNA Hybridization Chain Reaction (HCR™)

HCR™ RNA-FISH (v3.0) (Choi, H. M. T. et al. Development 145, dev165753 (2018)) was performed on thin brain tissue slices (20 μm) using commercial HCR™ buffers and HCR™ Amplifiers according to the manufacturer's instructions (Molecular Instruments). C57BL/6 mice (Jackson Laboratory, 000664, male, 10-13 weeks old) were used in the smFISH-HCR™ validation experiments. Briefly, tissue slices were fixed with 4% PFA in PBS on ice for 15 min, permeabilized with ice-cold methanol for 30 min, and washed with PBSTR (PBS with 0.1% Tween-20, 0.1 U/μL SUPERase-In) twice at room temperature for 10 min. The sample was then pre-incubated in the HCR™ Probe Hybridization Buffer at 37° C. for 10 min and then incubated at 37° C. for 12-16 hours overnight with custom-designed three or four pairs of HCR™ probes (final concentration of 25-100 nM for each probe) in the HCR™ Probe Hybridization Buffer supplemented with 1% Yeast tRNA and 0.1 U/μL SUPERase-In. The day after, the sample was washed with the HCR™ Probe Wash Buffer, and the signal was amplified with the HCR™ Amplifier probes at room temperature for 8-16 hours. The fluorescent amplification probe sets used included B1-Alexa647, B2-Alexa594, B3-Alexa546, and B5-Alexa488. Finally, the sample was washed with 5×SSCT, stained with DAPI, and imaged inside PBS with 10% SlowFade™ Gold Antifade Mountant with DAPI (Invitrogen, S36938) with Leica Stellaris 8.

### Imputation

Imputation of unmeasured genes was performed after integrating the scRNA-seq dataset and STARmap PLUS dataset, following a similar imputation strategy as in. Lohoff, T. et al. Nat. Biotechnol. 40, 74-85 (2022).

First, intermediate mapping was performed. Specifically, for each of the 1022 genes in the STARmap PLUS, an intermediate mapping was performed to align each STARmap PLUS cell with the most similar set of cells in the scRNA-seq dataset. The dimension reduction and batch effect correction methods were UMAP and Harmony. Here, the ‘leave-one-gene-out’ mapping approach was used to assess the performance changes caused by the number of nearest neighbors in scRNA-seq data. The performance score for each mapped gene was evaluated. The performance score was calculated as the Pearson correlation r (across cells) between its imputed values and measured STARmap PLUS expression level. According to the result in FIG. 64A, the number of nearest neighbors was chosen to be 200.

Finally, a final imputation was performed. First, the quality of the scRNA-seq data was checked: genes with average read<0.005/sum read<740 across 146,201 cells (50th percentile of the data) were filtered; genes with maximum read<=10 were filtered. It was found that 11,844 genes were left after the filtration, and these genes were then used for imputation. To perform imputation for all genes, aggregation was carried out across the intermediate mappings generated from each gene probed using STARmap PLUS. Specifically, for each STARmap PLUS cell, the set of all scRNA-seq atlas cells that were associated with the cell in any intermediate mapping was considered. Subsequently, for every cell, each gene's imputed expression level was calculated as the weighted average of the gene's expression across the associated set of scRNA-seq atlas cells, where weights were proportional to the number of times each scRNA-seq atlas cell was present (FIG. 55A). Thus, the imputed expression profiles for all genes, including those in the overlapping gene set, were on the same scale as the scRNA-seq log count data. The output was a 1,091,280 cell by 11,844 genes matrix. The performance score for the imputed genes was also evaluated by comparing them to Allen ISH data (Lein, E. S. et al. Nature 445, 168-176 (2007)). The performance score was calculated as the Pearson correlation r (across cells) between imputed values and measured STARmap PLUS expression level. Representative results are shown in FIGS. 55B and 64B-64C.

Using the genes with STARmap PLUS measured ground-truth, the following four gene expression features were examined for their association with the imputation performance in the “leave-one-out” intermediate imputation (FIGS. 64B and 69A-69D). Pearson correlation coefficient of each gene was calculated between intermediate mapping result and STARmap PLUS. (1) Gene expression level in STARmap PLUS. (2) Spatial expression heterogeneity in STARmap PLUS. For each gene, Moran's I (a coefficient measuring overall spatial autocorrelation) for the gene's spatial expression was calculated for each of the 20 sample slices by a function squidpy.gr.spatial_autocorr and then averaged, to represent the degree of patterned spatial expression. Higher Moran's I represented more patterned spatial gene expression. (3) Gene expression in scRNA-seq dataset. (4) Single-cell expression heterogeneity in scRNA-seq dataset. The degree of cell expression specificity of a gene was quantified by calculating Moran's I of the scRNA-seq UMAP colored by the gene's expression.

### Trajectory Analysis

Oligodendrocytes (OLG) and oligodendrocyte precursor cells (OPC) in main cluster annotation were extracted and their developmental trajectory was explored. These cells had subcluster annotations as OLG_1, OLG_2, OLG_3, and OPC.

To reconstruct differentiation trajectory, principal component analysis (PCA), neighbors, and diffusion maps were computed using functions scanpy.tl.pca, scanpy.pp.neighbors, and scanpy.tl.diffmap. Then, to quantify the connectivity of subcluster annotations of the single-cell graph, partition-based graph abstraction (PAGA) was used to generate a much simpler abstracted graph (PAGA graph) of partitions, in which edge weights represent confidence in the presence of connections using function scanpy.tl.diffmap. Next, to infer the progression of cells through geodesic distance along the graph, diffusion pseudotime was calculated with function scanpy.tl.dpt. The Scanpy package (scanpy.readthedocs.io/en/stable/index.html) was utilized for diffusion map and pseudotime calculation.

Cell-Type Cluster Correspondence with Brain Subregion scRNA-Seq Datasets

Specific regions were integrated with existing specialized single-cell datasets to examine the cross-dataset nomenclature correspondence for cell types.

First a scRNA-seq dataset in the mouse brain cortex and hippocampus was referred to (ref [portal.brain-map.org/atlases-and-data/rnaseq]). STARmap PLUS cells labeled in top-level tissue regions ‘CTX_A’, ‘CTX_B’, ‘L1_HPFmo_MNG’, ‘CTX_HIP_CA’, ‘CTX_HIP_DG’, and ‘ENTm’ were extracted. For integration of these STARmap PLUS cells and the scRNA-seq dataset, similar analyses were performed as described herein. First, Harmony was used to integrate all cells. Then the overlapped 1,021 genes between STARmap PLUS and scRNA-seq experiments was used to compute adjusted PC's and performed joint clustering to transfer cell-type labels in the scRNA-seq dataset to STARmap PLUS identified cells. The transferred labels for STARmap PLUS cells were decided based on the integration of STARmap PLUS cells with the scRNA-seq dataset. Within each joint cluster, the cell type labels of those scRNA-seq cells were checked. If the number of top-1 scRNA-seq cell-type labels within one joint cluster exceeded 60%, it indicated successful integration for multi-source single-cell datasets on this cell type. Therefore, this dominant top-1 scRNA-seq cell-type label was assigned to that joint cluster with high confidence. Otherwise, integration was regarded as unsuccessful and labels were not transferred from the scRNA-seq dataset to STARmap PLUS cells. The function scanpy.external.pp.harmony integrate was used to perform the integration. The function scanpy.tl.leiden was used with a resolution equal to 3 to perform joint clustering.

Then, similarly, an scRNA-seq dataset in mouse brain striatum and a scRNA-seq dataset in mouse cerebellum were referenced and the same analysis was performed to get correspondence for cell types. For the striatum, cells labeled as top-level tissue region ‘STR’ were extracted. For the cerebellum, cells labeled as top-level tissue regions ‘CBX_1’ and ‘CBX_2’ were extracted.

### RNA Barcode Analysis

Assign Circular RNA Barcode Spots into Cells

Spot-calling of circular RNA barcode spots was first performed according to the same process as that in the STARmap PLUS data processing part. Then, in each tile, the DAPI signal was binarized and used it as a mask to remove circular RNA barcode reads outside the cell nucleus. Then the spots in each tile were stitched together based on tile location information. Next, circular RNA barcode spots were assigned into cells identified by endogenous genes. The Nearest Neighbors algorithm (k=1) was used to determine which RNA barcode amplicons were in which cells. sklearn.neighbors.NearestNeighbors was used to identify the mRNA spots closest to each RNA barcode spot. Finally, the total number of circular RNA barcodes were counted for each cell.

### Cell Type-Based Statistics

For each cell main and subtype cell cluster, summary statistics of the 2.5th, 25th, 50th, 75th, and 97.5th percentiles were computed using numpy.quantile to generate a boxplot of circular RNA barcode expression by cell type in both coronal and sagittal samples.

### Tissue Region-Based Statistics

The 2.5th, 25th, 50th, 75th, and 97.5th percentiles were similarly compared for each tissue region after grouping cells by the tissue regions as generated above.

### Statistical Analysis

Spearman's r and its P values (two-tailed) in FIGS. 66A-66D and Pearson's r and its P values (two-tailed) were calculated with GraphPad Prism Version 9.3.1. P values in FIGS. 69A-69D were calculated with two-sided Mann-Whitney-Wilcoxon tests by statannotations (version 0.4.4) using the function statannotations.Annotator.annotator.configure (test=‘Mann-Whitney’, text_format=‘star’, loc=‘outside’). **P<0.01, ***P<0.001, ****P<0.0001.

### Code Availability Statement

The following packages and software (McInnes, L., Preprint at arxiv.org/abs/1802.03426 (2018); Bradski, G. Dr Dobb's J. Softw. Tools 25, 120-125 (2000).; Goddard, T. D., et al. J. Struct. Biol. 157, 281-287 (2007); Hunter, J. D. Comput. Sci. Eng. 9, 90-95 (2007); Virtanen, P. et al. Nat. Methods 17, 261-272 (2020); MacQueen, J. B. In Proc. of the fifth Berkeley Symposium on Mathematical Statistics and Probability, p. 281-297 (University of California Press, 1967); Higham, D. J. & Higham, N. J. MATLAB Guide, p. 150 (Siam, 2016); McKinney, W. In Proc. 9th Python in Science Conference (eds van der Walt, S. & Millman, J.) 51-56 (SciPy, 2010); Pedregosa, F. et al. Scikit-learn: machine learning in Python. J. Mach. Learn. Res 12, 2825-2830 (2011); Perez, F., et al. Comput. Sci. Eng. 13, 13-21 (2011); Heideman, M., IEEE ASSP Magazine. Vol. 1, p. 14-21 (IEEE, 1984); van der Walt, S. et al. scikit-image: image processing in Python. Peer J. 2, e453 (2014)) were used in the data analysis: ClusterMap was implemented based on MATLAB R2019b and Python 3.6. The following packages and software were used in data analysis: UCSF ChimeraX 1.0, ImageJ 1.51, MATLAB R2019b, R 4.0.4, Rstudio 1.4.1106, Jupyter Notebook 6.0.3, Anaconda 2-2-.02, h5py 3.1.0, hdbscan 0.8.36, hdf5 1.10.4, matplotlib 3.1.3, seaborn 0.11.0, scanpy 1.6.0, numpy 1.19.4, scipy 1.6.3, pandas 1.2.3, scikit-learn 0.22, umap-learn0.4.3, pip 21.0.1, numba 0.51.2, tifffile 2020.10.1, scikit-image 0.18.1, itertools 8.0.0. The code that supports the analyses in the examples is available at github.com/wanglab-broad/mCNS-atlas.

### Sample Preparation and Damage Evaluation

### STARmap PLUS Tissue Collection

During STARmap PLUS tissue sample collection, the whole mouse brain was freshly collected shortly after rapid decapitation (<5 min), embedded in OCT, flash-frozen in liquid nitrogen (˜10 minutes), and kept at −80° C. until brain slice sectioning (FIG. 66A). The brain tissues were sectioned at −20° C. with a cryostat, adhered to a coverslip, and immediately fixed with 4% paraformaldehyde (PFA) in PBS. The tissue samples were processed in frozen format until PFA fixation to minimize disturbance to the tissue and degradation of RNA, which can be reflected by the lower percentage of activated microglia in the whole microglia population (Ccl3⁺ or Ccl4⁺, 8.8% in the current atlas versus 24.6% in the scRNA-seq atlas).

Tissue sectioning could result in cell fragments at the slice surface. However, the STARmap PLUS method included the three following steps of quality control to address this issue: (i) small cell fragments without clear nuclear DAPI staining were filtered out; (ii) small cell fragments containing fewer than 30 reads or fewer than 20 genes were further filtered out; and (iii) variation brought by cell volume is normalized by counts per cell during pre-processing before cell clustering.

### Cell Clusters Quality Check

The number of reads and number of genes was compared among subclusters (FIGS. 66B-66D). First, a high correlation was observed between the median genes per cell and the median reads per cell among subclusters (FIG. 66B), indicating consistent detection efficiency among genes. Furthermore, there was no correlation between the cluster size (whether in terms of the number of cells in the subcluster, FIG. 66C; or the subcluster's population percentage within its main cluster, FIG. 66D) and the number of reads per cell or the number of genes per cell, thereby ruling out the possibility that small clusters were a result of low-quality cells caused by tissue damage or RNA degradation during sample preparation.

### Sequences

Tables 1A and 1B provide a list of plasmids used in the above examples, as well as gene insert sequences of the plasmids. In Table 1A:
lowercase bold text indicates a sequence encoding an epitope tag (e.g., FLAG or V5); UPPERCASE, ALL CAPS, BOLD TEXT indicates a sequence encoding a GGGGSₙ linker, where n is 1 or 2 (SEQ ID NO: 51);
lowercase italic text indicates a sequence encoding a nuclear export signal (NES) or a 3× nuclear localization signal (NLS);
lowercase, bold, underlined text indicates a sequence encoding an RNA binding domain (e.g., λN, MS2cp, PP7cp);
UPPERCASE ALL CAPS DASHED UNDERLINE TEXT indicates a sequence encoding an RNA motif capable of being bound by an RNA binding domain (e.g., BoxB, MS2, PP7; italic lowercase underline text indicates a sequence encoding a farnesylation motif (Far);
ALL CAPS, BOLD, ITALIC, UNDERLINE TEXT indicates a sequence encoding a myristoylation signal peptide (Myr);
lowercase, bold, italic, underline text indicates a sequence encoding a palmitoylation motif (Pal);
lowercase, bold, dashed underline text indicates a sequence encoding part of a three-way junction;
ALL CAPS, ITALIC DASHED UNDERLINE TEXT indicates a sequence encoding a barcode region with flanking cloning sites;
lowercase, double underlined text indicates a sequence encoding a self-cleaving ribozyme;
bold, double-underline, lowercase text indicates a sequence encoding a stem forming region;
lowercase, bold, underlined, italic text indicates a sequence encoding a self-cleaving peptide (e.g., T2A);
lowercase italic text indicates a promoter region (e.g., U6 or U6+27); the term “T6” indicates a stretch of 6 T's;
ALL CAPS UNDERLINED TEXT indicates a minihelix;
ALL CAPS ITALIC TEXT indicates a sequence encoding an M9 motif, DDX39A, or RtcB.

Tables 2A and 2B provide a list of promoter sequences used in the Examples.

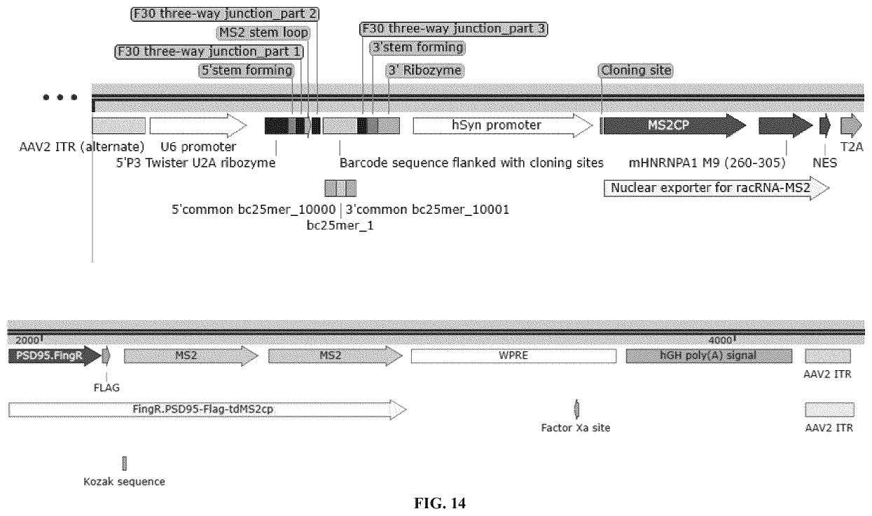

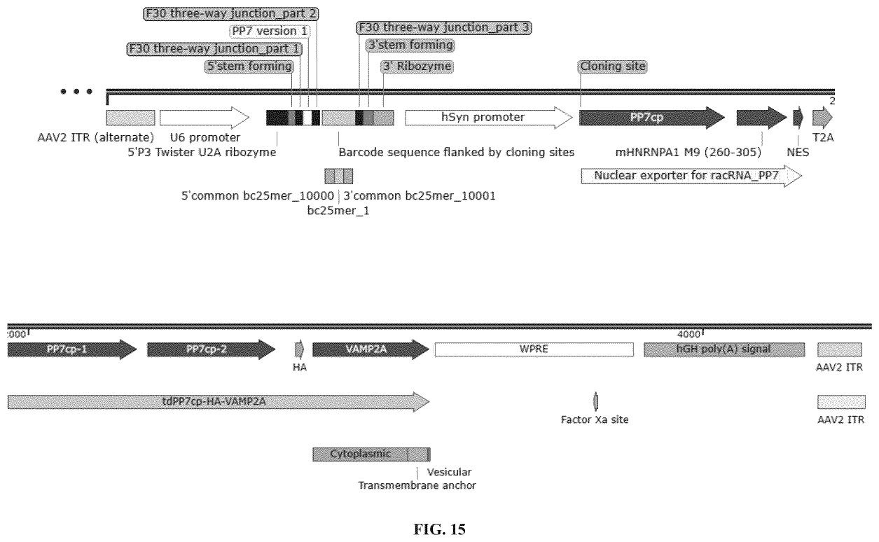

FIGS. 14A to 18B present annotated sequences for polypeptides and polynucleotides used in the examples (e.g., plasmid sequences and racRNA sequences encoded thereby).

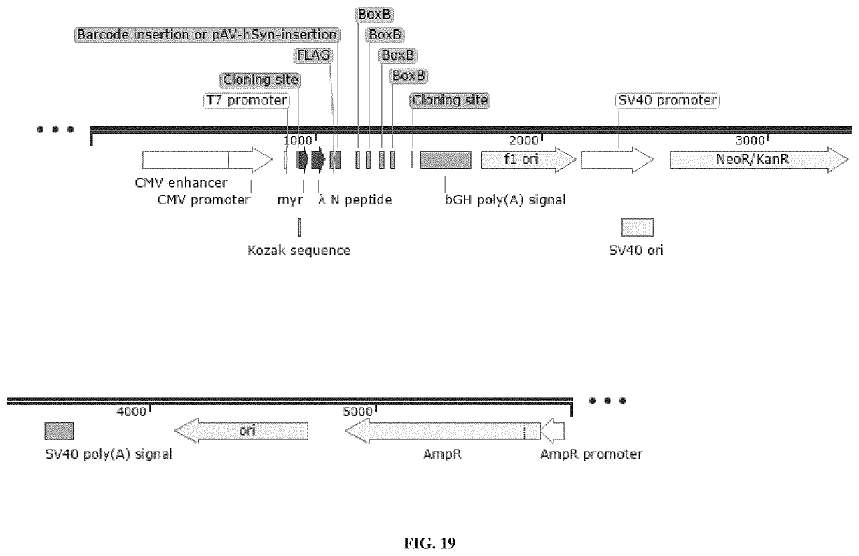

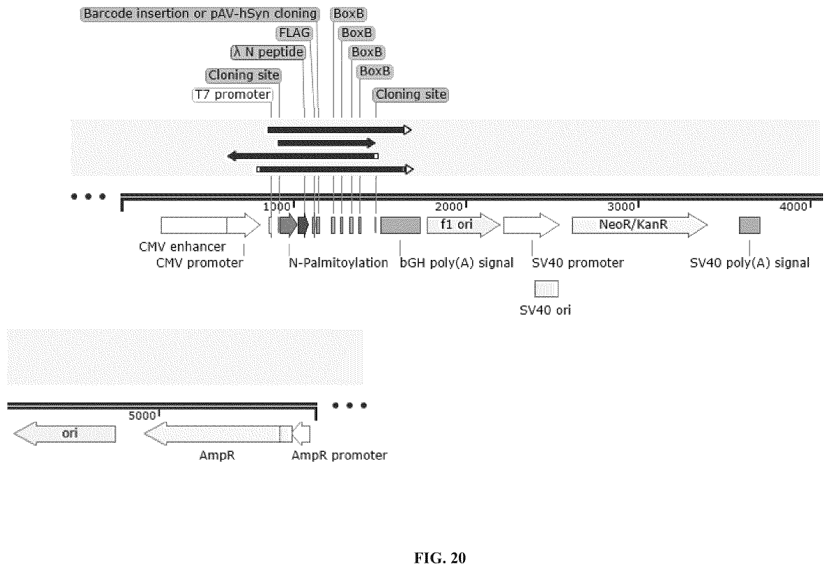

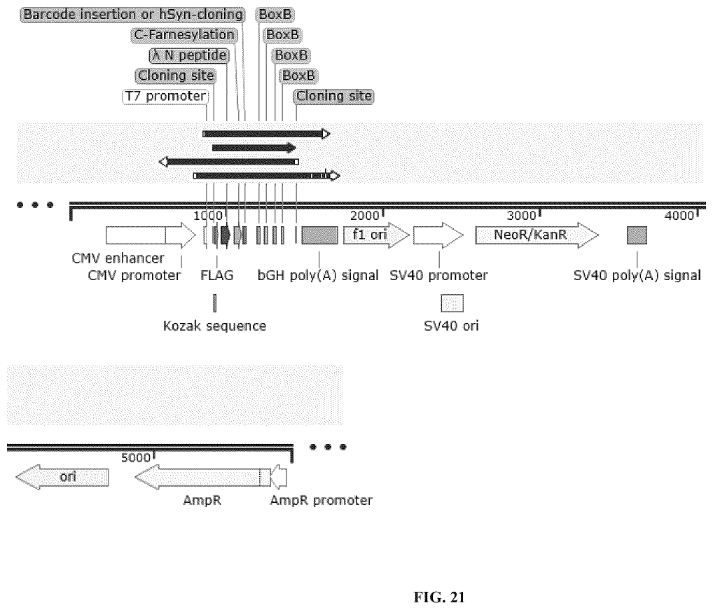

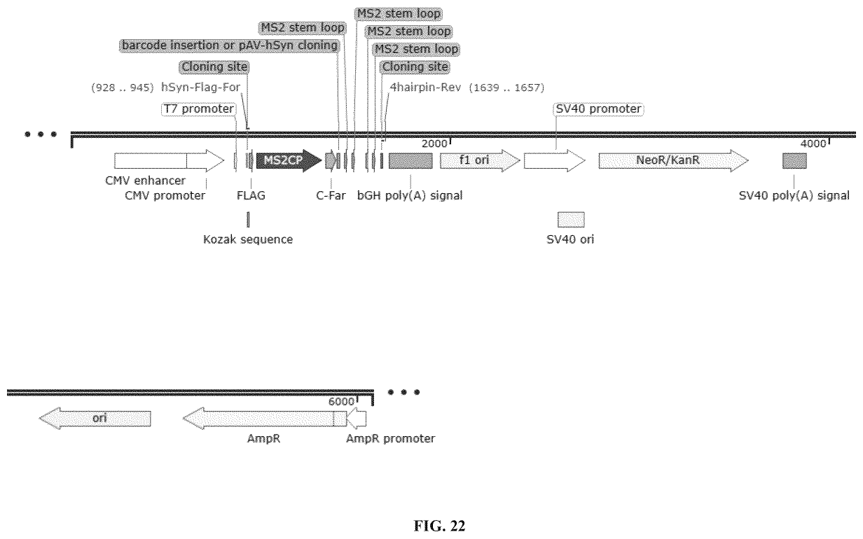

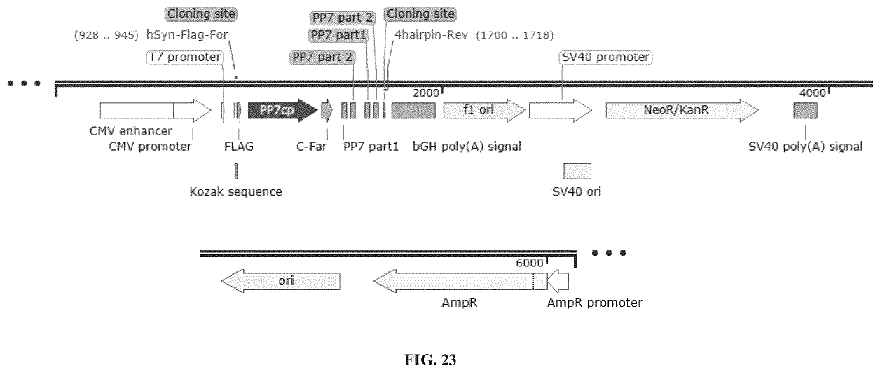

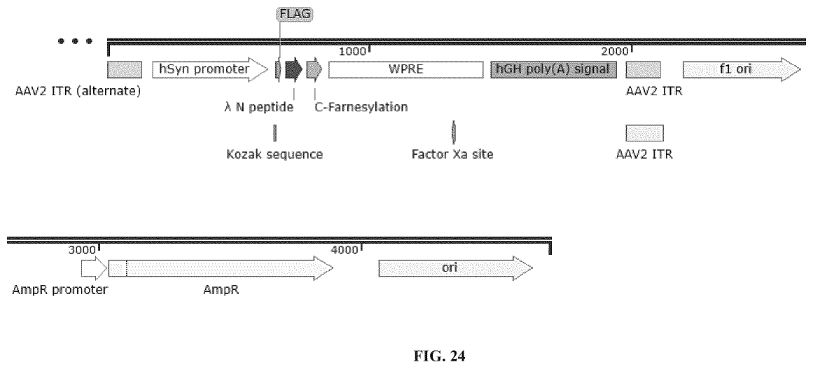

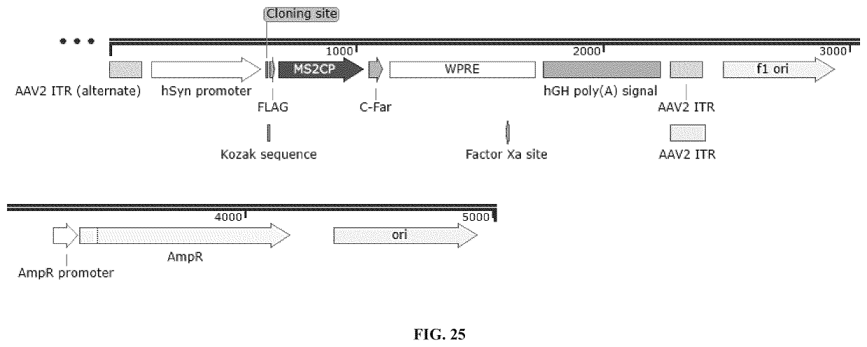

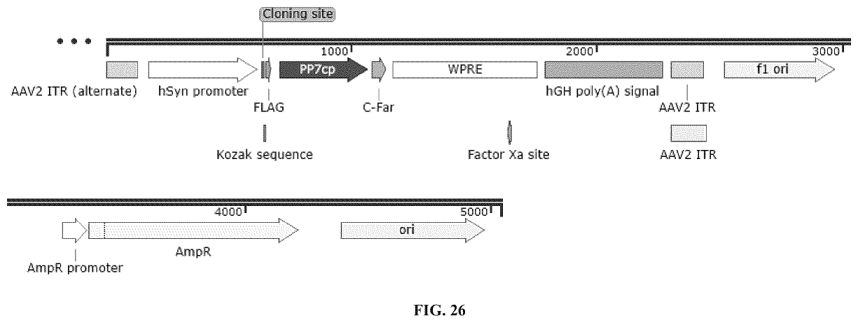

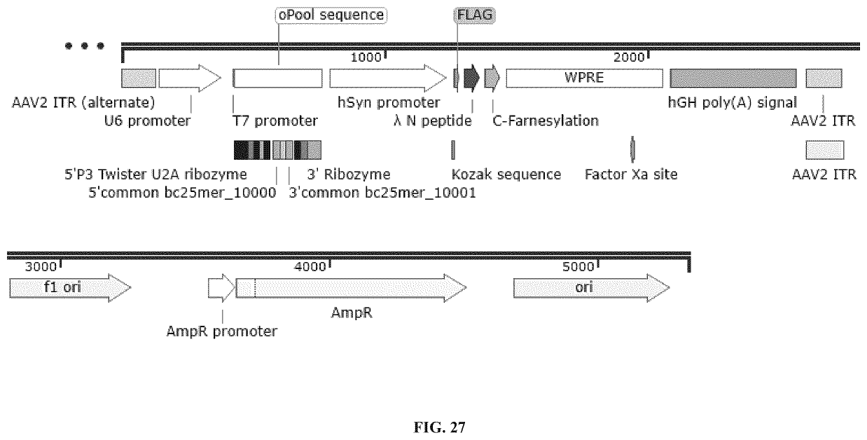

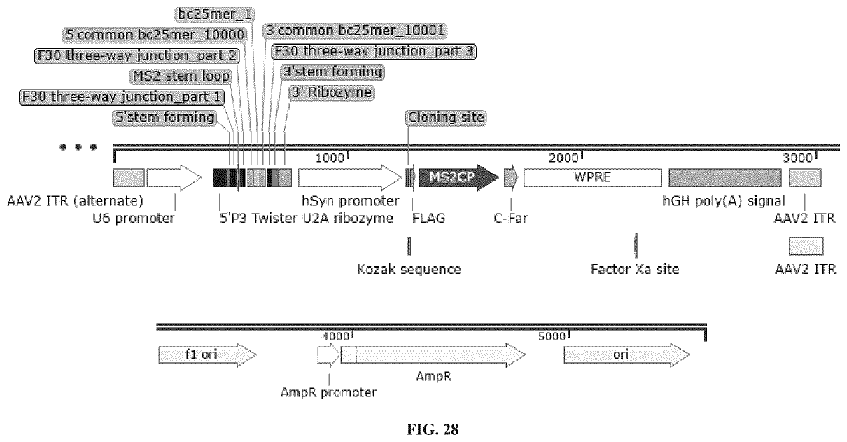

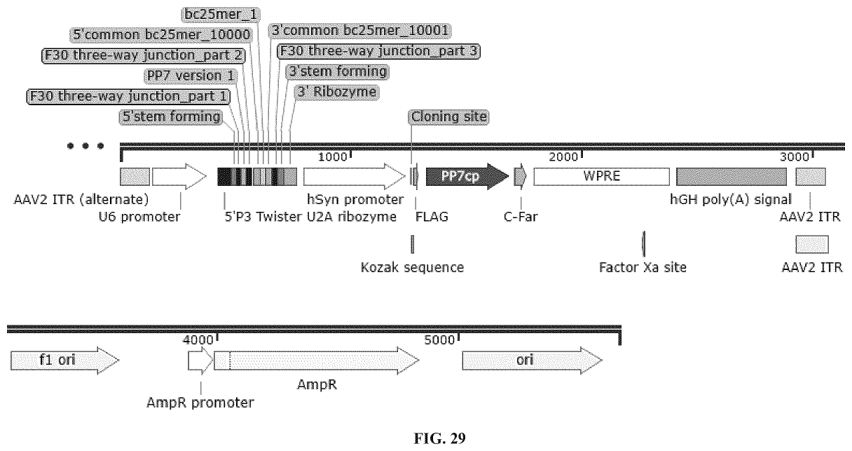

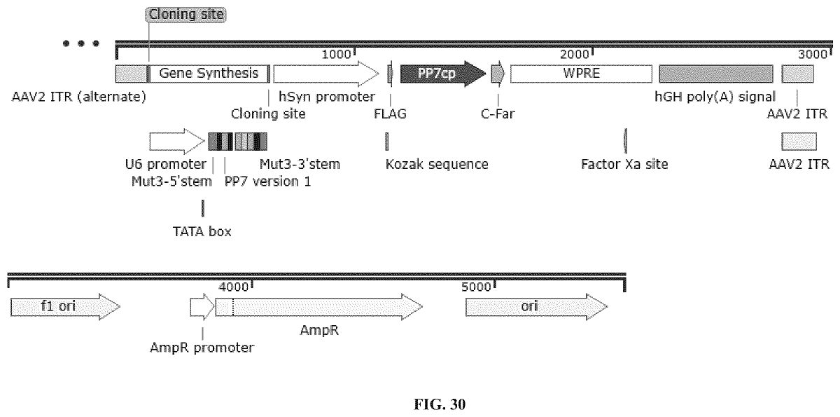

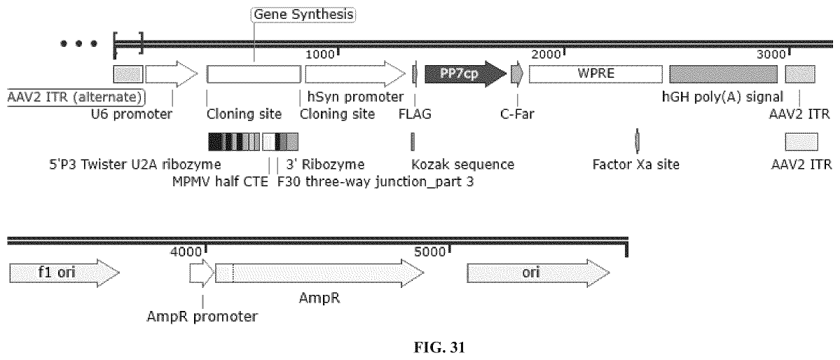

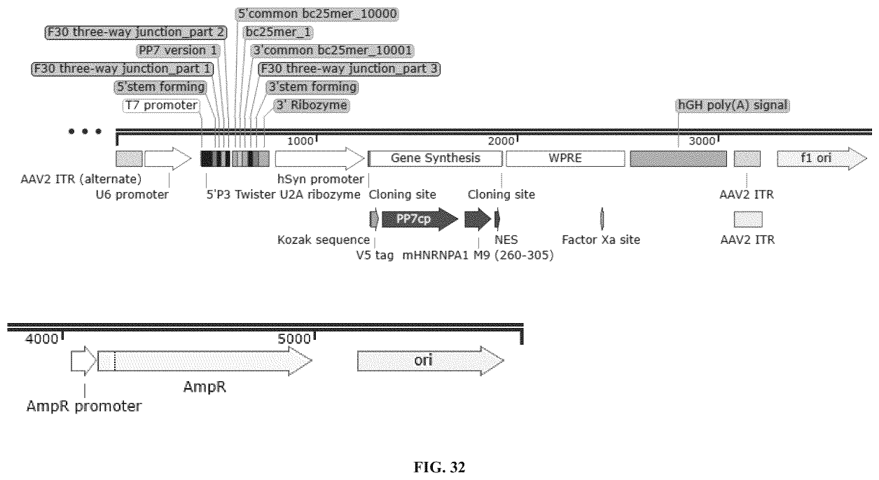

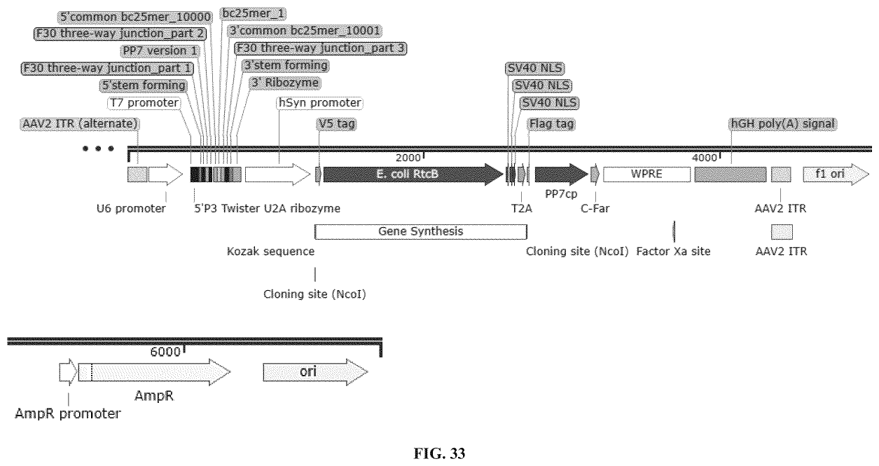

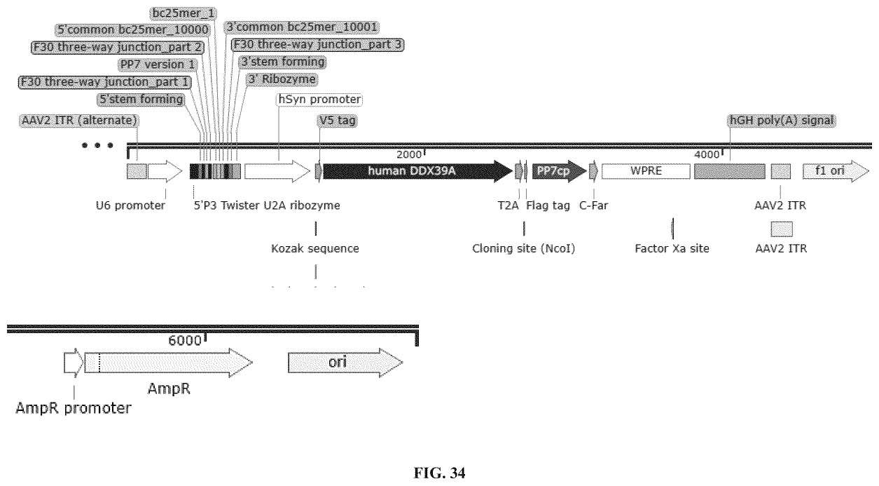

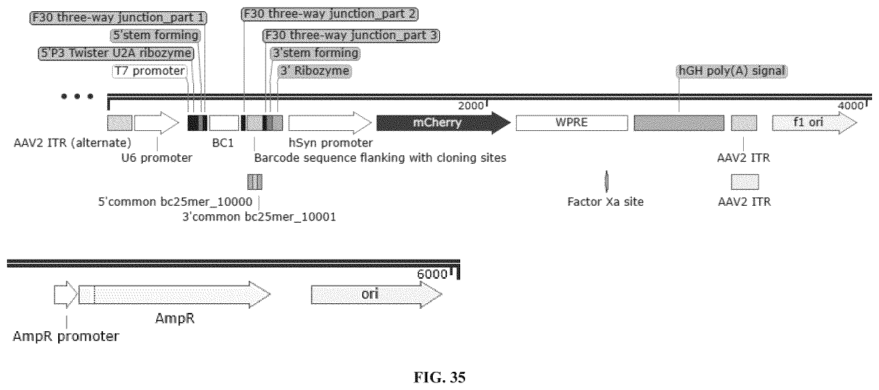

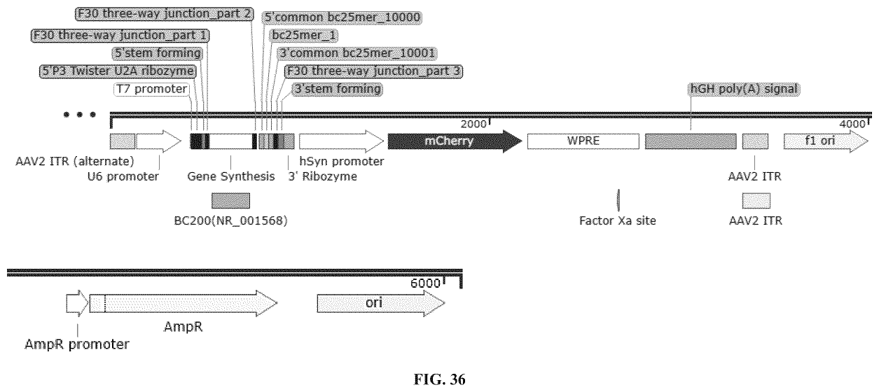

TABLE 1A
Plasmid sequences.

| Plasmid # | Gene Insert sequence |
|---|---|
| 1 (see | GCCACCATGGGGTCTTCAAAATCTAAACCAAAGGACCCCAGCCAGCGCGGCGGAGGTGGTTCT |
| FIG. 19) | gacgcacaaacacgacgacgtgagcgtcgcgctgagaaacaagctcaatggaaagctgcaaacGGCGGAGGTGGT |
| | TCTgattacaaggatgacgacgataagtaaGAATTCTGCAGATATCGCTCGCTTTCTTGCTGTCCAATTTC |
| | 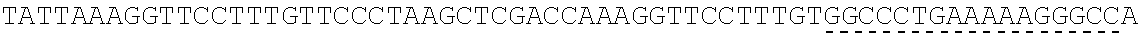 |
| | 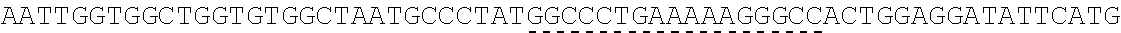 |
| | 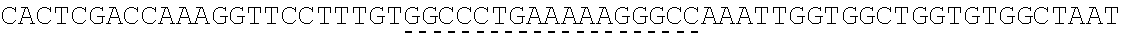 |
| | 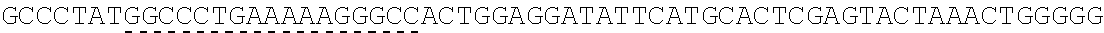 |
| | ATATTATGAAGGGCCTTGAGCATCTGGATTCTGCCTAAT (SEQ ID NO: 52) |
| 2 (see | GCCACCAtgctgtgctgcatcagaagaactaaaccggttgagaagaatgaagaggccgatcaggagctgcagtcg |
| FIG. 20) | acggtgccgcgggcccgggatccaccggtcgccaccgacgcacaaacacgacgacgtgagcgtcgcgctgagaaacaag |
| | ctcaatggaaagctgcaaacGGCGGAGGTGGTTCTgattacaaggatgacgacgataagtaaGAATTCTGCAG |
| | ATATCGCTCGCTTTCTTGCTGTCCAATTTCTATTAAAGGTTCCTTTGTTCCCTAAGCTCGACCAAAG |
| | 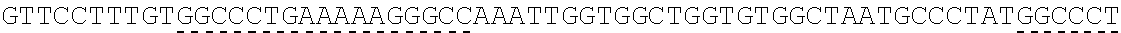 |
| | 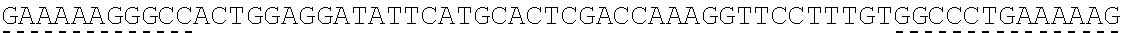 |
| | 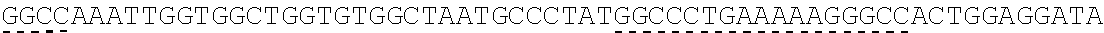 |
| | TTCATGCACTCGAGTACTAAACTGGGGGATATTATGAAGGGCCTTGAGCATCTGGATTCTGCCTA |
| | AT (SEQ ID NO: 53) |
| 3 (see | GCCACCatggattacaaggatgacgacgataagGGCGGAGGTGGTTCTgacgcacaaacacgacgacgtgagcg |
| FIG. 21) | tcgcgctgagaaacaagctcaatggaaagctgcaaacGGCGGAGGTGGTTCTaagctgaaccctcctgatgagag |
| | tggccccggctgcatgagctgctgtgtgctctcctaaGAATTCTGCAGATATCGCTCGCTTTCTTGCTGTCCAA |
| | 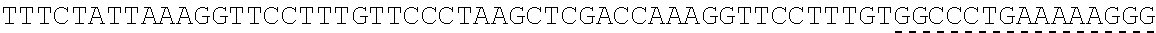 |
| | 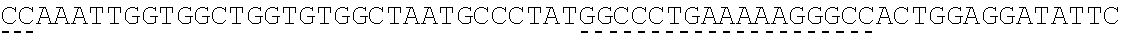 |
| | 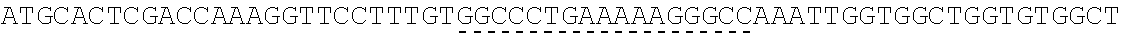 |
| | 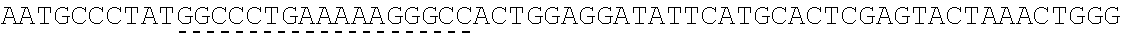 |
| | GGATATTATGAAGGGCCTTGAGCATCTGGATTCTGCCTAAT (SEQ ID NO: 54) |
| 4 (see | GCCACCatggattacaaggatgacgacgataagGGCGGAGGTGGTTCTgcttctaactttactcagttcgttctcgt |
| FIG. 22) | cgacaatggcggaactggcgacgtgactgtcgccccaagcaacttcgctaacggggtcgctgaatggatcagctctaactcg |
| | cgttcacaggcttacaaagtaacctgtagcgttcgtcagagctctgcgcagaatcgcaaatacaccatcaaagtcgaggtgc |
| | ctaaaggcgcatggcgttcgtacttaaatatggaactaaccattccaattttctccacgaactccgactgcgagcttattgtta |
| | aggcaatgcaaggtctcctaaaagatggaaacccgattccctcagcaatcgcagcaaactccggcatctacGGCGGAG |
| | GTGGTTCTaagctgaaccctcctgatgagagtggccccggctgcatgagctgctgtgtgctctcctaaGAATTCTGC |
| | 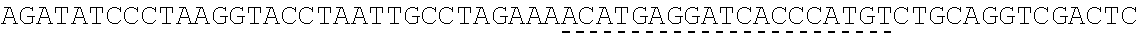 |
| | 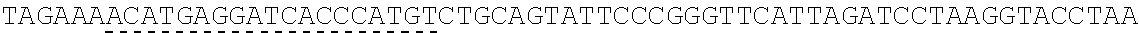 |
| | 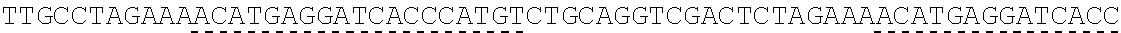 |
| | 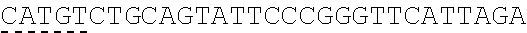 |
| | (SEQ ID NO: 55) |
| 5 (see | GCCACCatggattacaaggatgacgacgataagGGCGGAGGTGGTTCTtccaaaaccatcgttctttcggtcggcg |
| FIG. 23) | aggctactcgcactctgactgagatccagtccaccgcagaccgtcagatcttcgaagagaaggtcgggcctctggtgggtcg |
| | gctgcgcctcacggcttcgctccgtcaaaacggagccaagaccgcgtatcgcgtcaacctaaaactggatcaggcggacgtc |
| | gttgattgctccaccagcgtctgcggcgagcttccgaaagtgcgctacactcaggtatggtcgcacgacgtgacaatcgttgc |
| | gaatagcaccgaggcctcgcgcaaatcgttgtacgatttgaccaagtccctcgtcgcgacctcgcaggtcgaagatcttgtcg |
| | tcaaccttgtgccgctgggccgtGGCGGAGGTGGTTCTaagctgaaccctcctgatgagagtggccccggctgcatg |
| | 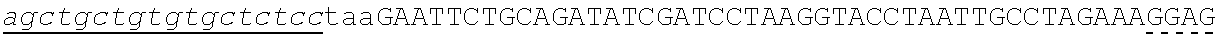 |
| | 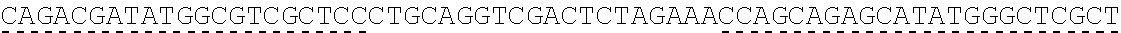 |
| | 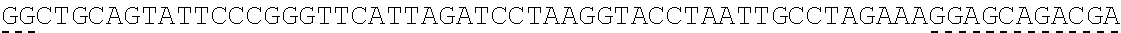 |
| | 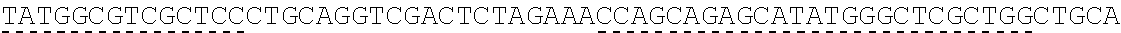 |
| | GTATTCCCGGGTTCATTAGAT (SEQ ID NO: 56) |
| 6 (see | GCCACCatggattacaaggatgacgacgataagGGCGGAGGTGGTTCTgacgcacaaacacgacgacgtgagcg |
| FIG. 24) | tcgcgctgagaaacaagctcaatggaaagctgcaaacGGCGGAGGTGGTTCTaagctgaaccctcctgatgagag |
| | tggccccggctgcatgagctgctgtgtgctctcctaa (SEQ ID NO: 57) |
| 7 (see | GCCACCatggattacaaggatgacgacgataagGGCGGAGGTGGTTCTgcttctaactttactcagttcgttctcgt |
| FIG. 25) | cgacaatggcggaactggcgacgtgactgtcgccccaagcaacttcgctaacggggtcgctgaatggatcagctctaactcg |
| | cgttcacaggcttacaaagtaacctgtagcgttcgtcagagctctgcgcagaatcgcaaatacaccatcaaagtcgaggtgc |
| | ctaaaggcgcatggcgttcgtacttaaatatggaactaaccattccaattttctccacgaactccgactgcgagcttattgtta |
| | aggcaatgcaaggtctcctaaaagatggaaacccgattccctcagcaatcgcagcaaactccggcatctacGGCGGAG |
| | GTGGTTCTaagctgaaccctcctgatgagagtggccccggctgcatgagctgctgtgtgctctcctaa (SEQ ID NO: 58) |
| 8 (see | GCCACCatggattacaaggatgacgacgataagGGCGGAGGTGGTTCTtccaaaaccatcgttctttcggtcggcg |
| FIG. 26) | aggctactcgcactctgactgagatccagtccaccgcagaccgtcagatcttcgaagagaaggtcgggcctctggtgggtcg |
| | gctgcgcctcacggcttcgctccgtcaaaacggagccaagaccgcgtatcgcgtcaacctaaaactggatcaggcggacgtc |
| | gttgattgctccaccagcgtctgcggcgagcttccgaaagtgcgctacactcaggtatggtcgcacgacgtgacaatcgttgc |
| | gaatagcaccgaggcctcgcgcaaatcgttgtacgatttgaccaagtccctcgtcgcgacctcgcaggtcgaagatcttgtcg |
| | tcaaccttgtgccgctgggccgtGGCGGAGGTGGTTCTaagctgaaccctcctgatgagagtggccccggctgcatg |
| | agctgctgtgtgctctcctaa (SEQ ID NO: 59) |
| 9 (see | gagggcctatttcccatgattccttcatatttgcatatacgatacaaggctgttagagagataattggaattaatttgactgt |
| FIG. 27) | aaacacaaagatattagtacaaaatacgtgacgtagaaagtaataatttcttgggtagtttgcagttttaaaattatgttt |
| | taaaatggactatcatatgcttaccgtaacttgaaagtatttcgatttcttggctttatatatcttgtggaaaggacgaaac |
| | accgtgctcgcttcggcagcacatatactagtcgacgggccgcactcgccggtcccaagcccggataaaatgggagggggc |
| | 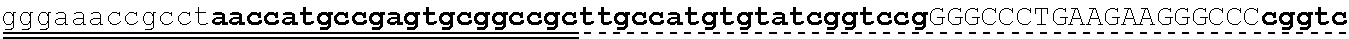 |
| | 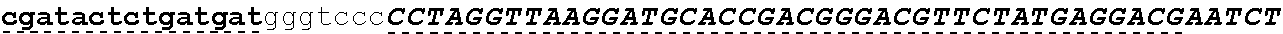 |
| | 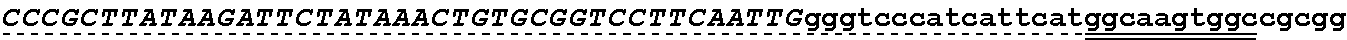 |
| | tcggcgtggactgtagaacactgccaatgccggtcccaagcccggataaaaGTGGAGGGTACAGTCCACGCtttttt |
| | (SEQ ID NO: 60) |
| 10 (see | gagggcctatttcccatgattccttcatatttgcatatacgatacaaggctgttagagagataattggaattaatttgactgt |
| FIG. 28) | aaacacaaagatattagtacaaaatacgtgacgtagaaagtaataatttcttgggtagtttgcagttttaaaattatgttt |
| | taaaatggactatcatatgcttaccgtaacttgaaagtatttcgatttcttggctttatatatcttgtggaaaggacgaaac |
| | accgtgctcgcttcggcagcacatatactagtcgacgggccgcactcgccggtcccaagcccggataaaatgggagggggc |
| | 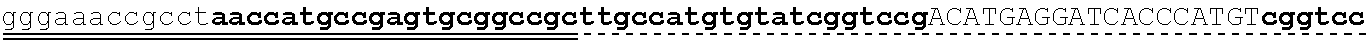 |
| | 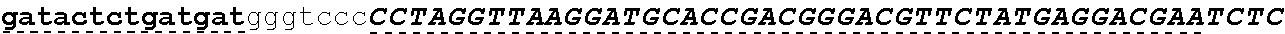 |
| | 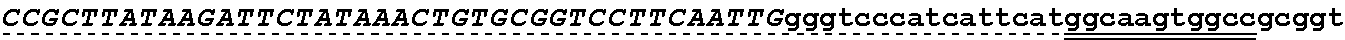 |
| | cggcgtggactgtagaacactgccaatgccggtcccaagcccggataaaaGTGGAGGGTACAGTCCACGCtttttt |
| | (SEQ ID NO: 61) |
| 11 (see | gagggcctatttcccatgattccttcatatttgcatatacgatacaaggctgttagagagataattggaattaatttgactgt |
| FIG. 29) | aaacacaaagatattagtacaaaatacgtgacgtagaaagtaataatttcttgggtagtttgcagttttaaaattatgttt |
| | taaaatggactatcatatgcttaccgtaacttgaaagtatttcgatttcttggctttatatatcttgtggaaaggacgaaac |
| | accgtgctcgcttcggcagcacatatactagtcgacgggccgcactcgccggtcccaagcccggataaaatgggagggggc |
| | 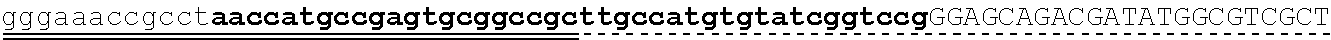 |
| | 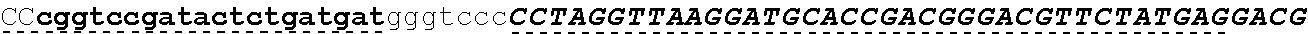 |
| | 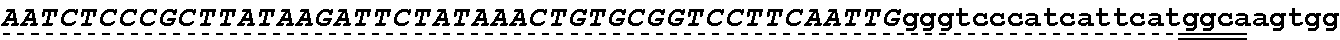 |
| | ccgcggtcggcgtggactgtagaacactgccaatgccggtcccaagcccggataaaaGTGGAGGGTACAGTCCACG |
| | Ctttttt (SEQ ID NO: 62) |
| 12 (see | gagggcctatttcccatgattccttcatatttgcatatacgatacaaggctgttagagagataattggaattaatttgactgt |
| FIG. 30) | aaacacaaagatattagtacaaaatacgtgacgtagaaagtaataatttcttgggtagtttgcagttttaaaattatgttt |
| | taaaatggactatcatatgcttaccgtaacttgaaagtatttcgatttcttggctttatatatcttgtggaaaggacgaaac |
| | 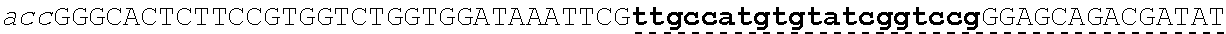 |
| | 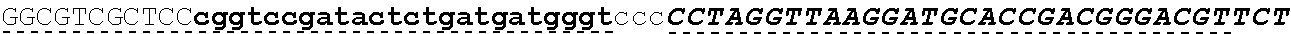 |
| | 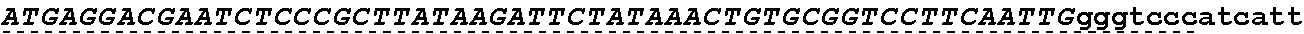 |
| | 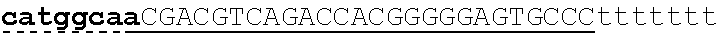 |
| | (SEQ ID NO: 63) |
| 13 (see | cgacgggccgcactcgccggtcccaagcccggataaaatgggagggggcgggaaaccgcctaaccatgccgagtgcggccg |
| FIG. 31) | 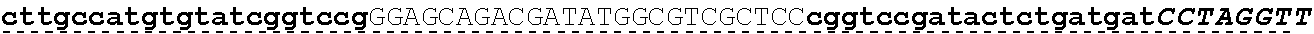 |
| | 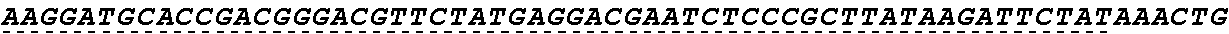 |
| | 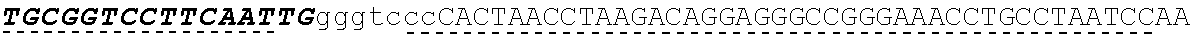 |
| | 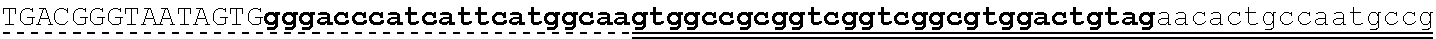 |
| | gtcccaagcccggataaaaGTGGAGGGTACAGTCCACGCtttttt (SEQ ID NO: 64) |
| 14 (see | GCCACCatgggcaagcccatccccaaccccctgctgggcctggacagcaccGGCGGTGGAGGTTCCtccaaaacc |
| FIG. 32) | atcgttctttcggtcggcgaggctactcgcactctgactgagatccagtccaccgcagaccgtcagatcttcgaagagaaggtc |
| | gggcctctggtgggtcggctgcgcctcacggcttcgctccgtcaaaacggagccaagaccgcgtatcgcgtcaacctaaaact |
| | ggatcaggcggacgtcgttgattgctccaccagcgtctgcggcgagcttccgaaagtgcgctacactcaggtatggtcgcacga |
| | cgtgacaatcgttgcgaatagcaccgaggcctcgcgcaaatcgttgtacgatttgaccaagtccctcgtcgcgacctcgcaggt |
| | cgaagatcttgtcgtcaaccttgtgccgctgggccgtGGCGGTGGCGGATCTGGCGGCGGTGGTAGCAATGA |
| | TTTTGGCAATTACAACAATCAGTCTTCCAATTTTGGGCCGATGAAGGGAGGAAACTTTGGAGGCA |
| | GGAGCTCTGGCCCTTATGGTGGTGGAGGCCAGTACTTTGCTAAACCACGGAACCAAGGTGGCTA |
| | TGGCGGAGGTGGTTCTctgcctccacttgaaagactgacactgtaa (SEQ ID NO: 65) |
| 15 (see | CCatgggcaagcccatccccaaccccctgctgggcctggacagcaccggcagcggcAACTATGAGCTTTTGACCAC |
| FIG. 33) | TGAGAACGCTCCTGTTAAGATGTGGACAAAAGGCGTGCCTGTAGAGGCCGACGCTCGGCAGCA |
| | ACTCATTAACACCGCCAAGATGCCCTTTATTTTCAAGCATATTGCCGTGATGCCTGATGTCCATCTT |
| | GGTAAGGGTTCAACAATCGGGAGCGTCATCCCTACCAAGGGTGCCATCATTCCAGCCGCCGTAG |
| | GAGTAGATATTGGATGCGGCATGAACGCACTTAGAACAGCTCTGACCGCCGAGGATCTTCCCGA |
| | GAACCTCGCTGAACTGCGACAGGCAATCGAGACAGCAGTTCCTCACGGCAGAACCACAGGCAGG |
| | TGTAAGAGAGATAAGGGCGCATGGGAAAACCCCCCCGTGAATGTCGACGCAAAATGGGCAGAG |
| | TTGGAAGCTGGGTATCAATGGCTGACCCAAAAGTACCCACGGTTCCTCAATACTAATAACTATAA |
| | GCACCTTGGGACACTCGGAACCGGCAACCACTTCATAGAAATATGCCTGGACGAGTCAGATCAA |
| | GTTTGGATAATGCTCCACTCTGGTTCACGGGGCATTGGCAACGCTATAGGAACATACTTTATAGA |
| | CCTGGCCCAGAAAGAGATGCAAGAAACATTGGAAACTCTCCCAAGTAGGGACCTCGCTTACTTCA |
| | TGGAGGGAACTGAGTATTTCGATGATTATCTGAAAGCCGTAGCATGGGCACAGTTGTTCGCCTCC |
| | TTGAATAGGGATGCAATGATGGAGAATGTCGTCACTGCTCTTCAAAGTATCACCCAAAAAACAGT |
| | ACGCCAACCTCAGACTCTGGCAATGGAAGAGATCAACTGTCATCATAACTACGTACAAAAGGAA |
| | CAACACTTCGGCGAAGAGATCTATGTTACCCGGAAAGGGGCCGTCTCAGCTAGGGCAGGCCAAT |
| | ACGGCATAATCCCTGGCTCTATGGGTGCAAAAAGCTTCATAGTTCGAGGCCTTGGGAACGAGGA |
| | GAGCTTTTGTAGCTGTAGCCACGGGGCTGGTCGGGTGATGTCCCGGACTAAAGCTAAAAAATTG |
| | TTCTCTGTTGAGGACCAAATACGGGCTACCGCACACGTAGAATGCCGGAAGGACGCCGAGGTCA |
| | TCGACGAAATCCCTATGGCCTACAAGGACATTGACGCAGTTATGGCCGCACAGTCTGACCTGGTG |
| | GAAGTTATATATACACTGAGGCAAGTAGTATGTGTGAAGGGAtctggtggttctcccaagaagaagagg |
| | aaggtggaccccaagaagaagaggaaggtggaccccaagaagaagaggaaggtgggctcaggaggagagggca |
| | gaggaagtcttctaacatgcggtgacgtggaggagaatcccggccctg (SEQ ID NO: 66) |
| 16 (see | CCatgggcaagcccatccccaaccccctgctgggcctggacagcaccggcagcggcGCAGAACAGGATGTGGAAA |
| FIG. 34) | ACGATCTTTTGGATTACGATGAAGAGGAAGAGCCCCAGGCTCCTCAAGAGAGCACACCAGCTCC |
| | CCCTAAGAAAGACATCAAGGGATCCTACGTTTCCATCCACAGCTCTGGCTTCCGGGACTTTCTGCT |
| | GAAGCCGGAGCTCCTGCGGGCCATCGTGGACTGTGGCTTTGAGCATCCTTCTGAGGTCCAGCAT |
| | GAGTGCATTCCCCAGGCCATCCTGGGCATGGACGTCCTGTGCCAGGCCAAGTCCGGGATGGGCA |
| | AGACAGCGGTCTTCGTGCTGGCCACCCTACAGCAGATTGAGCCTGTCAACGGACAGGTGACGGT |
| | CCTGGTCATGTGCCACACGAGGGAGCTGGCCTTCCAGATCAGCAAGGAATATGAGCGCTTTTCC |
| | AAGTACATGCCCAGCGTCAAGGTGTCTGTGTTCTTCGGTGGTCTCTCCATCAAGAAGGATGAAGA |
| | AGTGTTGAAGAAGAACTGTCCCCATGTCGTGGTGGGGACCCCGGGCCGCATCCTGGCGCTCGTG |
| | CGGAATAGGAGCTTCAGCCTAAAGAATGTGAAGCACTTTGTGCTGGACGAGTGTGACAAGATGC |
| | TGGAGCAGCTGGACATGCGGCGGGATGTGCAGGAGATCTTCCGCCTGACACCACACGAGAAGC |
| | AGTGCATGATGTTCAGCGCCACCCTGAGCAAGGACATCCGGCCTGTGTGCAGGAAGTTCATGCA |
| | GGATCCAATGGAGGTGTTTGTGGACGACGAGACCAAGCTCACGCTGCACGGCCTGCAGCAGTAC |
| | TACGTCAAACTCAAAGACAGTGAGAAGAACCGCAAGCTCTTTGATCTCTTGGATGTGCTGGAGTT |
| | TAACCAGGTGATAATCTTCGTCAAGTCAGTGCAGCGCTGCATGGCCCTGGCCCAGCTCCTCGTGG |
| | AGCAGAACTTCCCGGCCATCGCCATCCACCGGGGCATGGCCCAGGAGGAGCGCCTGTCACGCTA |
| | TCAGCAGTTCAAGGATTTCCAGCGGCGGATCCTGGTGGCCACCAATCTGTTTGGCCGGGGGATG |
| | GACATCGAGCGAGTCAACATCGTCTTTAACTACGACATGCCTGAGGACTCGGACACCTACCTGCA |
| | CCGGGTGGCCCGGGCGGGTCGCTTTGGCACCAAAGGCCTAGCCATCACTTTTGTGTCTGACGAG |
| | AATGATGCCAAAATCCTCAATGACGTCCAGGACCGGTTTGAAGTTAATGTGGCAGAACTTCCAGA |
| | GGAAATCGACATCTCCACATACATCGAGCAGAGCCGGtctggtggttctgagggcagaggaagtcttcta |
| | acatgcggtgacgtggaggagaatcccggccctg (SEQ ID NO: 67) |
| 22 (see | gagggcctatttcccatgattccttcatatttgcatatacgatacaaggctgttagagagataattggaattaatttgactgt |
| FIG. 35) | aaacacaaagatattagtacaaaatacgtgacgtagaaagtaataatttcttgggtagtttgcagttttaaaattatgttt |
| | taaaatggactatcatatgcttaccgtaacttgaaagtatttcgatttcttggctttatatatcttgtggaaaggacgaaaca |
| | ccgtgctcgcttcggcagcacatatactagtcgacgggccgcactcgccggtcccaagcccggataaaatgggagggggcgg |
| | 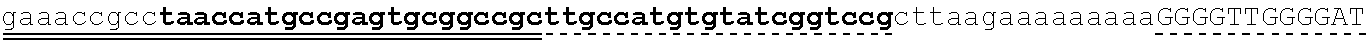 |
| | 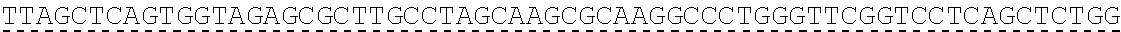 |
| | 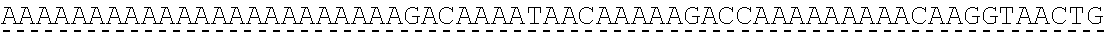 |
| | 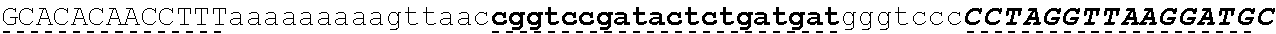 |
| | 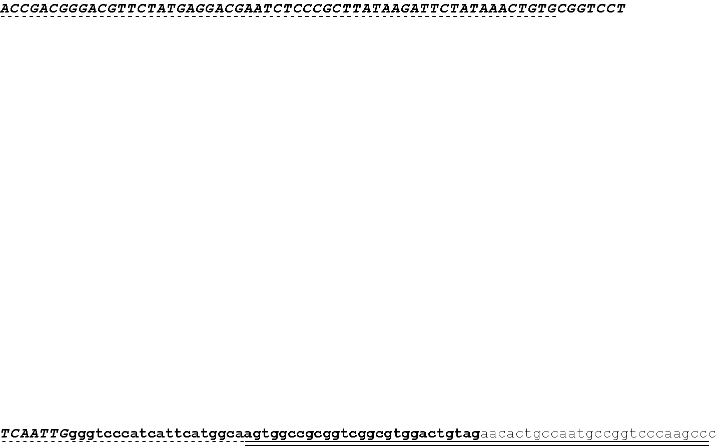 |
| | ggataaaaGTGGAGGGTACAGTCCACGCtttttt (SEQ ID NO: 68) |
| 23 (see | 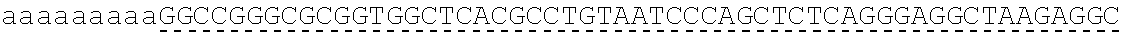 |
| FIG. 36) | 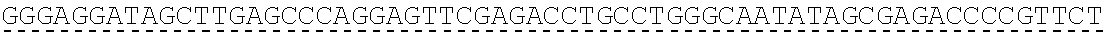 |
| | 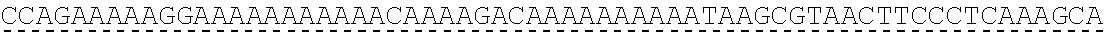 |
| | 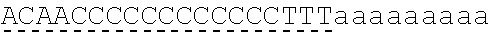 |
| | (SEQ ID NO: 69) |

TABLE 1B
Plasmid sequences.

| | | | Restriction | |
|---|---|---|---|---|
| Plasmid # | Plasmid name | Gene Insert Name | Enzyme sites | Vectors |
| 1 (see | pcDNA-Myr-λN-Flag- | Myr-λN-Flag- | KpnI, XbaI | pcDNA3.1(+) |
| FIG. 19) | 4BoxB | 4BoxB | | |
| 2 (see | pcDNA-Pal-λN-Flag-4BoxB | Pal-λN-Flag-4BoxB | KpnI, XbaI | pcDNA3.1(+) |
| FIG. 20) | | | | |
| 3 (see | pcDNA-Flag-λN-Far-4BoxB | Flag-λN-Far-4BoxB | KpnI, XbaI | pcDNA3.1(+) |
| FIG. 21) | | | | |
| 4 (see | pcDNA-Flag-MS2cp-Far- | Flag-MS2cp-Far- | BamHI, XhoI | pcDNA3.1(+) |
| FIG. 22) | 4MS2 | 4MS2 | | |
| 5 (see | pcDNA-Flag-PP7cp-Far- | Flag-PP7cp-Far- | BamHI, XhoI | pcDNA3.1(+) |
| FIG. 23) | 4PP7 | 4PP7 | | |
| 6 (see | pAAV-hSyn-Flag-λN-Far | Flag-λN-Far | KpnI, EcoRI | pAAV-hSyn- |
| FIG. 24) | | | | mCherry |
| | | | | (Addgene, |
| | | | | Plasmid |
| | | | | #114472) |
| 7 (see | pAAV-hSyn-Flag-MS2cp- | Flag-MS2cp-Far | BamHI, EcoRI | pAAV-hSyn- |
| FIG. 25) | Far | | | mCherry |
| | | | | (Addgene, |
| | | | | Plasmid |
| | | | | #114472) |
| 8 (see | pAAV-hSyn-Flag-PP7cp- | Flag-PP7cp-Far | BamHI, EcoRI | pAAV-hSyn- |
| FIG. 26) | Far | | | mCherry |
| | | | | (Addgene, |
| | | | | Plasmid |
| | | | | #114472) |
| 9 (see | pAAV-U6-racRNA-BoxB- | U6 + 27-racRNA- | MluI, XbaI | #6 pAAV-hSyn- |
| FIG. 27) | hSyn-Flag-λN-Far | BoxB-T6 | | Flag-λN-Far |
| 10 (see | pAAV-U6-racRNA-MS2- | U6 + 27-racRNA- | MluI, XbaI | #7 pAAV-hSyn- |
| FIG. 28) | hSyn-Flag-MS2cp-Far | MS2-T6 | | Flag-MS2cp-Far |
| 11 (see | pAAV-U6-racRNA-PP7- | U6 + 27-racRNA- | MluI, XbaI | #8 pAAV-hSyn- |
| FIG. 29) | hSyn-Flag-PP7cp-Far | PP7-T6 | | Flag-PP7cp-Far |
| 12 (see | pAAV-U6-linear-PP7-hSyn- | U6-terminal- | MluI, XbaI | #8 pAAV-hSyn- |
| FIG. 30) | Flag-PP7cp-Far | minihelix-T6 | | Flag-PP7cp-Far |
| 13 (see | pAAV-U6-racRNA-PP7- | racRNA-PP7-hCTE- | SpeI, XbaI | #11 pAAV-U6- |
| FIG. 31) | hCTE-hSyn-Flag-PP7cp-Far | T6 | | racRNA-PP7- |
| | | | | hSyn-Flag- |
| | | | | PP7cp-Far |
| 14 (see | pAAV-U6-racRNA-PP7- | V5-PP7cp- | BamHI, EcoRI | #11 pAAV-U6- |
| FIG. 32) | hSyn-V5-PP7cp-M9-NES | (GGGGS)2-M9- | | racRNA-PP7- |
| | | GGGGS-NES | | hSyn-Flag- |
| | | | | PP7cp-Far |
| 15 (see | pAAV-U6-racRNA-PP7- | V5-RtcB-3XNLS- | NcoI | #11 pAAV-U6- |
| FIG. 33) | hSyn-V5-RtcB-3XNLS- | T2A | | racRNA-PP7- |
| | T2A-Flag-PP7cp-Far | | | hSyn-Flag- |
| | | | | PP7cp-Far |
| 16 (see | pAAV-U6-racRNA-PP7- | V5-DDX39A-T2A | NcoI | #11 pAAV-U6- |
| FIG. 34) | hSyn-V5-DDX39A-T2A- | | | racRNA-PP7- |
| | Flag-PP7cp-Far | | | hSyn-Flag- |
| | | | | PP7cp-Far |
| 22 (see | pAAV-U6-racBC1-hSyn- | U6-racBC1-T6 | MluI, XbaI | pAAV-hSyn- |
| FIG. 35) | mCherry | | | mCherry |
| | | | | (Addgene, |
| | | | | Plasmid |
| | | | | #114472) |
| 23 (see | pAAV-U6-racBC200-hSyn- | racBC200 | AflII, HpaI | #22pAAV-U6- |
| FIG. 36) | mCherry | | | racBC1-hSyn- |
| | | | | mCherry |

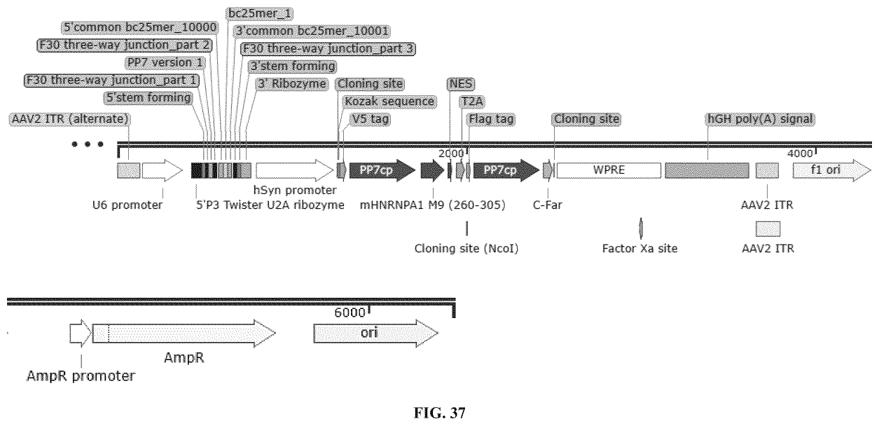

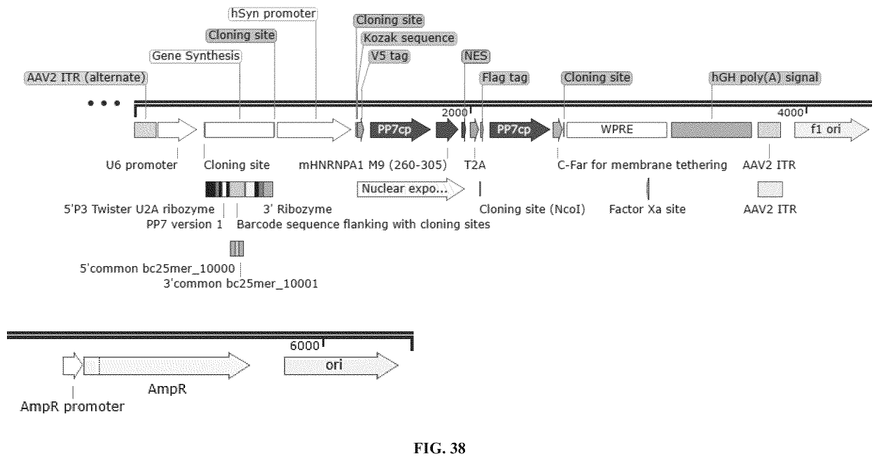

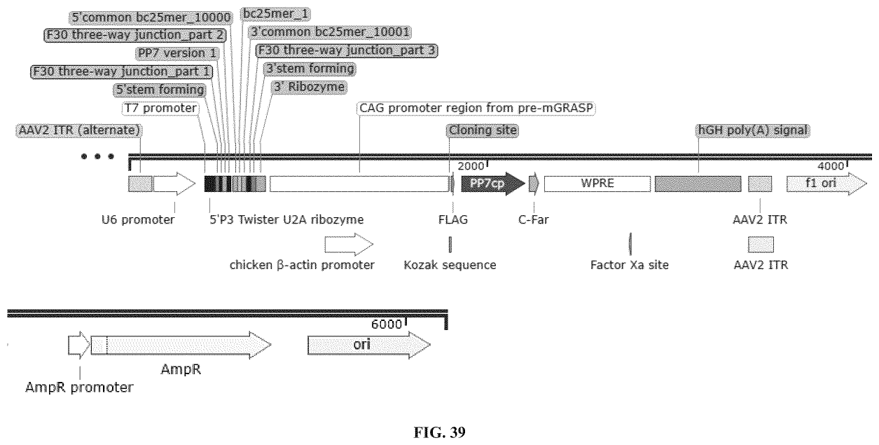

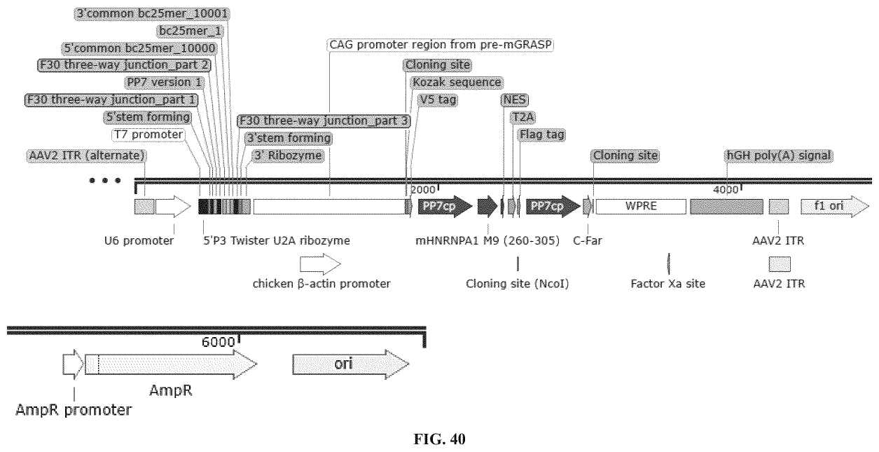

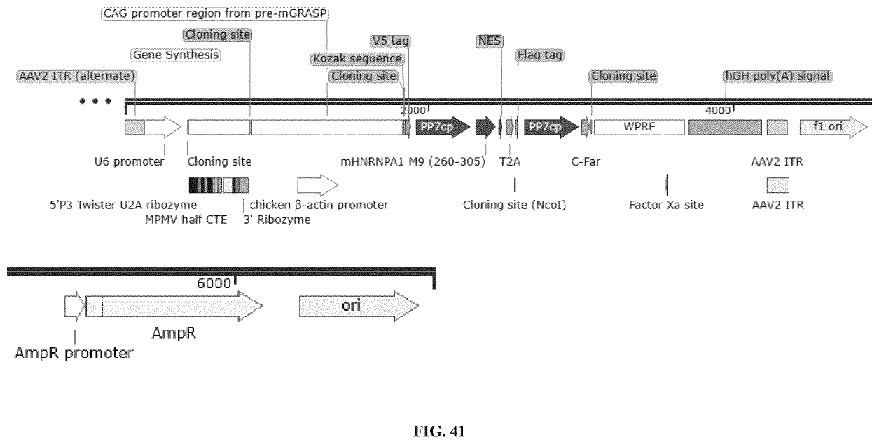

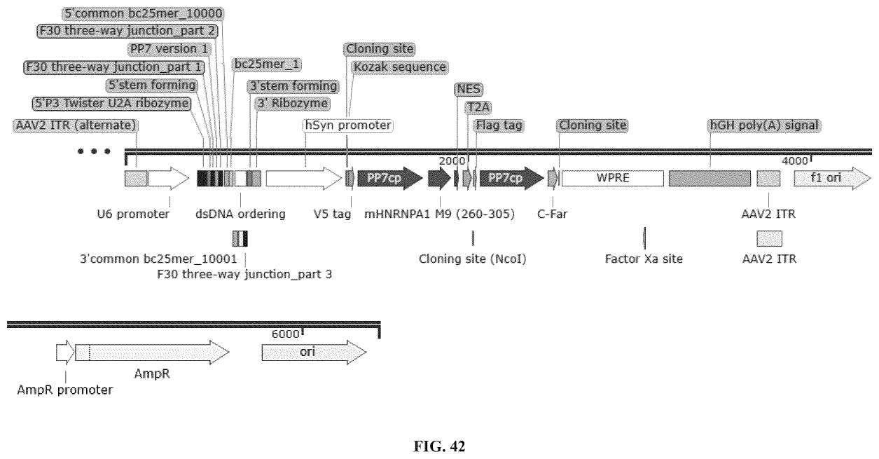

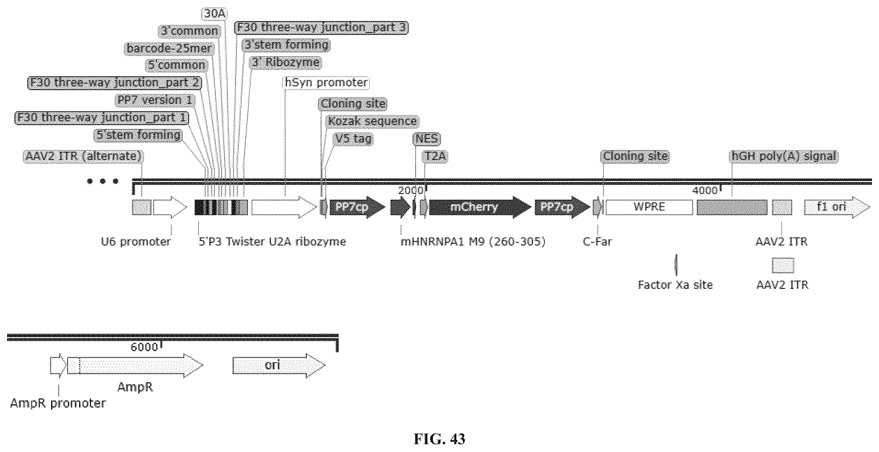

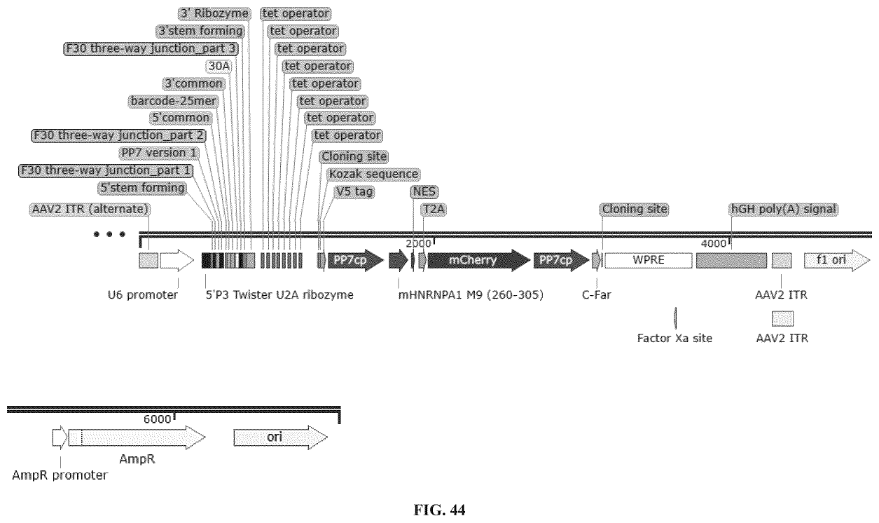

TABLE 2A
Primer sequences.

| Plasmid # | PCR forward primer | PCR reverse primer |
|---|---|---|
| 17 (see | ccacttgaaagactgaca | gtaatccagaggttgatt |
| FIG. 37) | ctgggctcaggaggatct | atcgataagcttg |
| | ggtggttctgagggcag | (SEQ ID NO: 71) |
| | (SEQ ID NO: 70) | |
| 18 (see | ccacttgaaagactgaca | gtaatccagaggttgatt |
| FIG. 38) | ctgggctcaggaggatct | atcgataagcttg |
| | ggtggttctgagggcag | (SEQ ID NO: 71) |
| | (SEQ ID NO: 70) | |
| 19 (see | CACGCtttttttctagac | ggcttgcccatGGTGGCG |
| FIG. 39) | tgcagagggcccTAATGA | GATCCAATTCTTTGCCAA |
| | TTAACCCGCCATGCTACT | AATGATG |
| | TATC | (SEQ ID NO: 73) |
| | (SEQ ID NO: 72) | |
| 20 (see | CACGCtttttttctagac | ggcttgcccatGGTGGCG |
| FIG. 40) | tgcagagggcccTAATGA | GATCCAATTCTTTGCCAA |
| | TTAACCCGCCATGCTACT | AATGATG |
| | TATC | (SEQ ID NO: 73) |
| | (SEQ ID NO: 72) | |
| 21 (see | CACGCtttttttctagac | ggcttgcccatGGTGGCG |
| FIG. 41) | tgcagagggcccTAATGA | GATCCAATTCTTTGCCAA |
| | TTAACCCGCCATGCTACT | AATGATG |
| | TATC | (SEQ ID NO: 73) |
| | (SEQ ID NO: 72) | |
| 24 (see | TAAACTGTGCGGTCCTTC | ccatgaatgatgggaccc |
| FIG. 42) | AATTGAAAAAAAAAAAAA | TTTTTTTTTTTTTTTTTT |
| | AAAAAAAAAAAAAAAAAg | TTTTTTTTTTTTCAATTG |
| | ggtcccatcattcatgg | AAGGACCGCACAGTTTA |
| | (SEQ ID NO: 74) | (SEQ ID NO: 75) |
| 25 (see | gcgcagtcgagaaggtac | cttgctcaccatGGcagg |
| FIG. 43) | c | gccggg |
| | (SEQ ID NO: 76) | (SEQ ID NO: 77) |
| | cctgCCatggtgagcaag | ACCTCCGCCcttgtacag |
| | ggcgagga | ctcgtccatgcc |
| | (SEQ ID NO: 78) | (SEQ ID NO: 79) |
| | ctgtacaagGGCGGAGGT | aggttgattatcgataag |
| | GGTTCTtcc | cttgatatcg |
| | (SEQ ID NO: 80) | (SEQ ID NO: 81) |
| 26 (see | Gctttttttctagactgc | cttgcccatGGTGGCGGA |
| FIG. 44) | agagggccctcaagtgcc | TCCaggctggatcgg |
| | acctgacgtctcc | (SEQ ID NO: 83) |
| | (SEQ ID NO: 82) | |

TABLE 2B
Primer sequences.

| | | | | | Restriction |
|---|---|---|---|---|---|
| | | Gene | | | Enzyme sites |
| Plasmid | | Insert | | | for vector |
| # | Plasmid name | Name | PCR template | Vectors | linearization |
| 17 (see | pAAV-U6- | T2A- | #15 pAAV-U6- | #11 pAAV-U6- | HindIII |
| FIG. 37) | racRNA-PP7- | Flag- | racRNA-PP7-hSyn- | racRNA-PP7- | |
| | hSyn-V5-PP7cp- | PP7cp- | V5-RtcB-3XNLS- | hSyn-Flag-PP7cp- | |
| | M9-NES-Flag- | Far | T2A-Flag-PP7cp- | Far | |
| | PP7cp-Far | | Far | | |
| 18 (see | pAAV-U6- | T2A- | #15 pAAV-U6- | #13 pAAV-U6- | HindIII |
| FIG. 38) | racRNA-PP7- | Flag- | racRNA-PP7-hSyn- | racRNA-PP7- | |
| | hCTE-hSyn-V5- | PP7cp- | V5-RtcB-3XNLS- | hCTE-hSyn-Flag- | |
| | PP7cp-M9-NES- | Far | T2A-Flag-PP7cp- | PP7cp-Far | |
| | Flag-PP7cp-Far | | Far | | |
| 19 (see | pAAV-U6- | CAG | paavCAG-pre- | #11 pAAV-U6- | ApaI, BamHI |
| FIG. 39) | racRNA-PP7- | promoter | mGRASP-2A- | racRNA-PP7- | |
| | CAG-Flag- | | dTomato (Addgene | hSyn-Flag-PP7cp- | |
| | PP7cp-Far | | Plasmid #51902) | Far | |
| 20 (see | pAAV-U6- | CAG | paavCAG-pre- | #17 pAAV-U6- | ApaI, BamHI |
| FIG. 40) | racRNA-PP7- | promoter | mGRASP-2A- | racRNA-PP7- | |
| | CAG-V5-PP7cp- | | dTomato (Addgene | hSyn-V5-PP7cp- | |
| | M9-NES-Flag- | | Plasmid #51902) | M9-NES-Flag- | |
| | PP7cp-Far | | | PP7cp-Far | |
| 21 (see | pAAV-U6- | CAG | paavCAG-pre- | #18 pAAV-U6- | ApaI, BamHI |
| FIG. 41) | racRNA-PP7- | promoter | mGRASP-2A- | racRNA-PP7- | |
| | hCTE-CAG-V5- | | dTomato (Addgene | hCTE-hSyn-V5- | |
| | PP7cp-M9-NES- | | Plasmid #51902) | PP7cp-M9-NES- | |
| | Flag-PP7cp-Far | | | Flag-PP7cp-Far | |
| 24 (see | pAAV-U6- | 30A | N.A. (oligo | #17 pAAV-U6- | MfeI |
| FIG. 42) | racRNA-PP7- | | annealing) | racRNA-PP7- | |
| | 30A-hSyn-V5- | | | hSyn-V5-PP7cp- | |
| | PP7cp-M9-NES- | | | M9-NES-Flag- | |
| | Flag-PP7cp-Far | | | PP7cp-Far | |
| 25 (see | pAAV-U6- | mCherry | # 24 pAAV-U6- | # 24 pAAV-U6- | BamHI, EcoRI |
| FIG. 43) | racRNA-PP7- | | racRNA-PP7-30A- | racRNA-PP7- | |
| | 30A-hSyn-V5- | | hSyn-V5-PP7cp- | 30A-hSyn-V5- | |
| | PP7cp-M9-NES- | | M9-NES-Flag- | PP7cp-M9-NES- | |
| | mCherry-PP7cp- | | PP7cp-Far | Flag-PP7cp-Far | |
| | Far | | pAAVpAAV- | | |
| | | | hSyn-mCherry | | |
| | | | (Addgene, Plasmid | | |
| | | | #114472) -hSyn- | | |
| | | | mCherry | | |
| | | | # 24 pAAV-U6- | | |
| | | | racRNA-PP7-30A- | | |
| | | | hSyn-V5-PP7cp- | | |
| | | | M9-NES-Flag- | | |
| | | | PP7cp-Far | | |
| 26 (see | pAAV-U6- | TRE | pAAV-TRE- | # 24 pAAV-U6- | ApaI, BamHI |
| FIG. 44) | racRNA-PP7- | | mRuby2 (Addgene | racRNA-PP7- | |
| | 30A-TRE-V5- | | Plasmid #99114) | 30A-hSyn-V5- | |
| | PP7cp-M9-NES- | | | PP7cp-M9-NES- | |
| | mCherry-PP7cp- | | | Flag-PP7cp-Far | |
| | Far | | | | |

The following are polynucleotide sequences of plasmids used in the examples:
Plasmid encoding racRNA-MS2-FingR-PSD95 (postsynapse) (see FIG. 14 for a map of the plasmid)

(SEQ ID NO: 84)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTGAGGGCCTATTTCCCATGATTCCTTCATATTTGCATATACGATACAAG
GCTGTTAGAGAGATAATTGGAATTAATTTGACTGTAAACACAAAGATATTAGTACAAAATACGT
GACGTAGAAAGTAATAATTTCTTGGGTAGTTTGCAGTTTTAAAATTATGTTTTAAAATGGACTA
TCATATGCTTACCGTAACTTGAAAGTATTTCGATTTCTTGGCTTTATATATCTTGTGGAAAGGA
CGAAACACCGTGCTCGCTTCGGCAGCACATATACTAGTCGACGGGCCGCACTCGCCGGTCCCAA
GCCCGGATAAAATGGGAGGGGGGGGGAAACCGCCTAACCATGCCGAGTGCGGCCGCTTGCCATG
TGTATCGGTCCGACATGAGGATCACCCATGTCGGTCCGATACTCTGATGATGGGTCCCCCTAGG
TTAAGGATGCACCGACGGGACGTTCTATGAGGACGAATCTCCCGCTTATAAGATTCTATAAACT
GTGCGGTCCTTCAATTGGGGTCCCATCATTCATGGCAAGTGGCCGCGGTCGGCGTGGACTGTAG
AACACTGCCAATGCCGGTCCCAAGCCCGGATAAAAGTGGAGGGTACAGTCCACGCTTTTTTTCT
AGACTGCAGAGGGCCCTGCGTATGAGTGCAAGTGGGTTTTAGGACCAGGATGAGGCGGGGTGGG
GGTGCCTACCTGACGACCGACCCCGACCCACTGGACAAGCACCCAACCCCCATTCCCCAAATTG
CGCATCCCCTATCAGAGAGGGGGAGGGGAAACAGGATGCGGCGAGGCGCGTGCGCACTGCCAGC
TTCAGCACCGCGGACAGTGCCTTCGCCCCCGCCTGGCGGCGCGCGCCACCGCCGCCTCAGCACT
GAAGGCGCGCTGACGTCACTCGCCGGTCCCCCGCAAACTCCCCTTCCCGGCCACCTTGGTCGCG
TCCGCGCCGCCGCCGGCCCAGCCGGACCGCACCACGCGAGGCGCGAGATAGGGGGGCACGGGCG
CGACCATCTGCGCTGCGGCGCCGGCGACTCAGCGCTGCCTCAGTCTGCGGTGGGCAGCGGAGGA
GTCGTGTCGTGCCTGAGAGCGCAGTCGAGAAGGTACCGGATCCGCCACCATGGCTTCTAACTTT
ACTCAGTTCGTTCTCGTCGACAATGGCGGAACTGGCGACGTGACTGTCGCCCCAAGCAACTTCG
CTAACGGGGTCGCTGAATGGATCAGCTCTAACTCGCGTTCACAGGCTTACAAAGTAACCTGTAG
CGTTCGTCAGAGCTCTGCGCAGAATCGCAAATACACCATCAAAGTCGAGGTGCCTAAAGGCGCA
TGGCGTTCGTACTTAAATATGGAACTAACCATTCCAATTTTCTCCACGAACTCCGACTGCGAGC
TTATTGTTAAGGCAATGCAAGGTCTCCTAAAAGATGGAAACCCGATTCCCTCAGCAATCGCAGC
AAACTCCGGCATCTACGGCGGTGGCGGATCTGGCGGCGGTGGTAGCAATGATTTTGGCAATTAC
AACAATCAGTCTTCCAATTTTGGGCCGATGAAGGGAGGAAACTTTGGAGGCAGGAGCTCTGGCC
CTTATGGTGGTGGAGGCCAGTACTTTGCTAAACCACGGAACCAAGGTGGCTATGGCGGAGGTGG
TTCTCTGCCTCCACTTGAAAGACTGACACTGGGCTCAGGAGGATCTGGTGGTTCTGAGGGCAGA
GGAAGTCTTCTAACATGCGGTGACGTGGAGGAGAATCCCGGCCCTGCCACCATGCTCGAAGTCA
AGGAAGCATCACCAACCAGCATCCAGATCAGCTGGGTGCTCCACTTGCGCCACGTTCGCTACTA
CCGCATCACCTACGGTGAAACTGGTGGCAATAGCCCTGTCCAGGAATTCACCGTGCCTGGCAGC
AAGTCCACTGCTACCATCAGCGGCCTGAAACCTGGTGTCGACTATACCATCACGGTGTACGCCG
TCACGATCTTCAGCGCCTACCGCTCCGCCTGGCCGCCGATCTCCATCAACTACCGCACCGGAAC
CGATTACAAGGATGACGACGATAAGGGTAGCGGCTCCAGTAGATCTGGGCTACTTAAGGCCACC
ATGGCCAGCAACTTCACCCAGTTTGTGCTGGTGGACAATGGCGGGACAGGCGATGTGACTGTGG
CTCCCTCCAACTTCGCCAATGGGGTGGCTGAGTGGATCAGCTCCAACAGTCGGTCACAGGCCTA
CAAGGTGACCTGCAGCGTGCGGCAGTCTAGTGCTCAGAAGAGAAAGTACACAATTAAGGTGGAG
GTGCCCAAAGTGGCCACCCAGACAGTGGGAGGAGTGGAACTGCCTGTGGCTGCTCGGAGATCCT
ACCTGAACATGGAGCTGACTATCCCTATTTTCGCCACCAATTCTGACTGTGAACTGATCGTGAA
GGCTATGCAGGGACTGCTGAAAGATGGCAACCCCATCCCTTCTGCCATTGCCGCTAATAGTGGA
ATCTATGGCGCCCCTGGGATTCACCCAGGGATGATGGCTTCTAACTTCACCCAATTTGTGCTGG
TCGATAATGGGGGAACCGGAGATGTGACAGTGGCCCCAAGTAACTTTGCCAATGGCGTGGCTGA
ATGGATCTCAAGCAATAGCCGGTCCCAGGCCTACAAAGTGACTTGCTCCGTGAGGCAGTCCTCT
GCTCAGAAGCGCAAATATACAATTAAGGTCGAAGTGCCAAAAGTGGCCACTCAGACCGTCGGCG
GAGTGGAACTGCCCGTGGCTGCTAGGCGCAGTTACCTGAATATGGAACTGACAATCCCTATTTT
TGCCACTAATTCAGATTGCGAGCTGATTGTGAAGGCTATGCAGGGGCTGCTGAAAGATGGAAAC
CCAATCCCCTCAGCCATTGCCGCTAATAGCGGCATCTACCTCGAGCCATAAGCTTATCGATAAT
CAACCTCTGGATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTATGGCTTTCAT
TTTCTCCTCCTTGTATAAATCCTGGTTGCTGTCTCTTTATGAGGAGTTGTGGCCCGTTGTCAGG
CAACGTGGCGTGGTGTGCACTGTGTTTGCTGACGCAACCCCCACTGGTTGGGGCATTGCCACCA
CCTGTCAGCTCCTTTCCGGGACTTTCGCTTTCCCCCTCCCTATTGCCACGGCGGAACTCATCGC
CGCCTGCCTTGCCCGCTGCTGGACAGGGGCTCGGCTGTTGGGCACTGACAATTCCGTGGTGTTG
TCGGGGAAATCATCGTCCTTTCCTTGGCTGCTCGCCTATGTTGCCACCTGGATTCTGCGCGGGA
CGTCCTTCTGCTACGTCCCTTCGGCCCTCAATCCAGCGGACCTTCCTTCCCGCGGCCTGCTGCC
GGCTCTGCGGCCTCTTCCGCGTCTTCGCCTTCGCCCTCAGACGAGTCGGATCTCCCTTTGGGCC
GCCTCCCCGCATCGATACCGAGCGCTGCTCGAGAGATCTACGGGTGGCATCCCTGTGACCCCTC
CCCAGTGCCTCTCCTGGCCCTGGAAGTTGCCACTCCAGTGCCCACCAGCCTTGTCCTAATAAAA
TTAAGTTGCATCATTTTGTCTGACTAGGTGTCCTTCTATAATATTATGGGGTGGAGGGGGGTGG
TATGGAGCAAGGGGCAAGTTGGGAAGACAACCTGTAGGGCCTGCGGGGTCTATTGGGAACCAAG
CTGGAGTGCAGTGGCACAATCTTGGCTCACTGCAATCTCCGCCTCCTGGGTTCAAGCGATTCTC
CTGCCTCAGCCTCCCGAGTTGTTGGGATTCCAGGCATGCATGACCAGGCTCAGCTAATTTTTGT
TTTTTTGGTAGAGACGGGGTTTCACCATATTGGCCAGGCTGGTCTCCAACTCCTAATCTCAGGT
GATCTACCCACCTTGGCCTCCCAAATTGCTGGGATTACAGGCGTGAACCACTGCTCCCTTCCCT
GTCCTTCTGATTTTGTAGGTAACCACGTGCGGACCGAGCGGCCGCAGGAACCCCTAGTGATGGA
GTTGGCCACTCCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGCTGCCTGCAGGGGCGCC
TGATGCGGTATTTTCTCCTTACGCATCTGTGCGGTATTTCACACCGCATACGTCAAAGCAACCA
TAGTACGCGCCCTGTAGCGGCGCATTAAGCGCGGCGGGTGTGGTGGTTACGCGCAGCGTGACCG
CTACACTTGCCAGCGCCTTAGCGCCCGCTCCTTTCGCTTTCTTCCCTTCCTTTCTCGCCACGTT
CGCCGGCTTTCCCCGTCAAGCTCTAAATCGGGGGCTCCCTTTAGGGTTCCGATTTAGTGCTTTA
CGGCACCTCGACCCCAAAAAACTTGATTTGGGTGATGGTTCACGTAGTGGGCCATCGCCCTGAT
AGACGGTTTTTCGCCCTTTGACGTTGGAGTCCACGTTCTTTAATAGTGGACTCTTGTTCCAAAC
TGGAACAACACTCAACTCTATCTCGGGCTATTCTTTTGATTTATAAGGGATTTTGCCGATTTCG
GTCTATTGGTTAAAAAATGAGCTGATTTAACAAAAATTTAACGCGAATTTTAACAAAATATTAA
CGTTTACAATTTTATGGTGCACTCTCAGTACAATCTGCTCTGATGCCGCATAGTTAAGCCAGCC
CCGACACCCGCCAACACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCACCGTCATCACCGAAAC
GCGCGAGACGAAAGGGCCTCGTGATACGCCTATTTTTATAGGTTAATGTCATGATAATAATGGT
TTCTTAGACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAATAATATT
GAAAAAGGAAGAGTATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATGCTGAAGATCAGTTG
GGTGCACGAGTGGGTTACATCGAACTGGATCTCAACAGCGGTAAGATCCTTGAGAGTTTTCGCC
CCGAAGAACGTTTTCCAATGATGAGCACTTTTAAAGTTCTGCTATGTGGCGCGGTATTATCCCG
TATTGACGCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATGCAGTGCTG
CCATAACCATGAGTGATAACACTGCGGCCAACTTACTTCTGACAACGATCGGAGGACCGAAGGA
GCTAACCGCTTTTTTGCACAACATGGGGGATCATGTAACTCGCCTTGATCGTTGGGAACCGGAG
CTGAATGAAGCCATACCAAACGACGAGCGTGACACCACGATGCCTGTAGCAATGGCAACAACGT
TGCGCAAACTATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGGTTTATTGCT
GATAAATCTGGAGCCGGTGAGCGTGGGTCTCGCGGTATCATTGCAGCACTGGGGCCAGATGGTA
AGCCCTCCCGTATCGTAGTTATCTACACGACGGGGAGTCAGGCAACTATGGATGAACGAAATAG
ACAGATCGCTGAGATAGGTGCCTCACTGATTAAGCATTGGTAACTGTCAGACCAAGTTTACTCA
TATATACTTTAGATTGATTTAAAACTTCATTTTTAATTTAAAAGGATCTAGGTGAAGATCCTTT
TTGATAATCTCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTTTTCCGA
AGGTAACTGGCTTCAGCAGAGCGCAGATACCAAATACTGTTCTTCTAGTGTAGCCGTAGTTAGG
CCACCACTTCAAGAACTCTGTAGCACCGCCTACATACCTCGCTCTGCTAATCCTGTTACCAGTG
GCTGCTGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAACGACCTA
CACCGAACTGAGATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTTTTTACGGTTC
CTGGCCTTTTGCTGGCCTTTTGCTCACATGT

Plasmid encoding racRNA-PP7-VAMP2A (see FIG. 15 for a map of the plasmid)

(SEQ ID NO: 85)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTGAGGGCCTATTTCCCATGATTCCTTCATATTTGCATATACGATACAAG
GCTGTTAGAGAGATAATTGGAATTAATTTGACTGTAAACACAAAGATATTAGTACAAAATACGT
GACGTAGAAAGTAATAATTTCTTGGGTAGTTTGCAGTTTTAAAATTATGTTTTAAAATGGACTA
TCATATGCTTACCGTAACTTGAAAGTATTTCGATTTCTTGGCTTTATATATCTTGTGGAAAGGA
CGAAACACCGTGCTCGCTTCGGCAGCACATATACTAGTCGACGGGCCGCACTCGCCGGTCCCAA
GCCCGGATAAAATGGGAGGGGGGGGGAAACCGCCTAACCATGCCGAGTGCGGCCGCTTGCCATG
TGTATCGGTCCGGGAGCAGACGATATGGCGTCGCTCCCGGTCCGATACTCTGATGATGGGTCCC
CCTAGGTTAAGGATGCACCGACGGGACGTTCTATGAGGACGAATCTCCCGCTTATAAGATTCTA
TAAACTGTGCGGTCCTTCAATTGGGGTCCCATCATTCATGGCAAGTGGCCGCGGTCGGCGTGGA
CTGTAGAACACTGCCAATGCCGGTCCCAAGCCCGGATAAAAGTGGAGGGTACAGTCCACGCTTT
TTTTCTAGACTGCAGAGGGCCCTGCGTATGAGTGCAAGTGGGTTTTAGGACCAGGATGAGGCGG
GGTGGGGGTGCCTACCTGACGACCGACCCCGACCCACTGGACAAGCACCCAACCCCCATTCCCC
AAATTGCGCATCCCCTATCAGAGAGGGGGAGGGGAAACAGGATGCGGCGAGGCGCGTGCGCACT
GCCAGCTTCAGCACCGCGGACAGTGCCTTCGCCCCCGCCTGGCGGCGCGCGCCACCGCCGCCTC
AGCACTGAAGGCGCGCTGACGTCACTCGCCGGTCCCCCGCAAACTCCCCTTCCCGGCCACCTTG
GTCGCGTCCGCGCCGCCGCCGGCCCAGCCGGACCGCACCACGCGAGGCGCGAGATAGGGGGGCA
CGGGCGCGACCATCTGCGCTGCGGCGCCGGCGACTCAGCGCTGCCTCAGTCTGCGGTGGGCAGC
GGAGGAGTCGTGTCGTGCCTGAGAGCGCAGTCGAGAAGGTACCGGATCCATGTCCAAAACCATC
GTTCTTTCGGTCGGCGAGGCTACTCGCACTCTGACTGAGATCCAGTCCACCGCAGACCGTCAGA
TCTTCGAAGAGAAGGTCGGGCCTCTGGTGGGTCGGCTGCGCCTCACGGCTTCGCTCCGTCAAAA
CGGAGCCAAGACCGCGTATCGCGTCAACCTAAAACTGGATCAGGCGGACGTCGTTGATTGCTCC
ACCAGCGTCTGCGGCGAGCTTCCGAAAGTGCGCTACACTCAGGTATGGTCGCACGACGTGACAA
TCGTTGCGAATAGCACCGAGGCCTCGCGCAAATCGTTGTACGATTTGACCAAGTCCCTCGTCGC
GACCTCGCAGGTCGAAGATCTTGTCGTCAACCTTGTGCCGCTGGGCCGTGGCGGTGGCGGATCT
GGCGGCGGTGGTAGCAATGATTTTGGCAATTACAACAATCAGTCTTCCAATTTTGGGCCGATGA
AGGGAGGAAACTTTGGAGGCAGGAGCTCTGGCCCTTATGGTGGTGGAGGCCAGTACTTTGCTAA
ACCACGGAACCAAGGTGGCTATGGCGGAGGTGGTTCTCTGCCTCCACTTGAAAGACTGACACTG
GGCTCAGGAGGATCTGGTGGTTCTGAGGGCAGAGGAAGTCTTCTAACATGCGGTGACGTGGAGG
AGAATCCCGGCCCTGCCACCATGTCCAAAACCATCGTTCTTTCGGTCGGCGAGGCTACTCGCAC
TCTGACTGAGATCCAGTCCACCGCAGACCGTCAGATCTTCGAAGAGAAGGTCGGGCCTCTGGTG
GGTCGGCTGCGCCTCACGGCTTCGCTCCGTCAAAACGGAGCCAAGACCGCGTATCGCGTCAACC
TAAAACTGGATCAGGCGGACGTCGTTGATTGCTCCACCAGCGTCTGCGGCGAGCTTCCGAAAGT
GCGCTACACTCAGGTATGGTCGCACGACGTGACAATCGTTGCGAATAGCACCGAGGCCTCGCGC
AAATCGTTGTACGATTTGACCAAGTCCCTCGTCGCGACCTCGCAGGTCGAAGATCTTGTCGTCA
ACCTTGTGCCGCTGGGCCGTCGTGCGGACCCGCTAGCCTCCTGCGGCCGCTCCAAAACCATCGT
TCTTTCGGTCGGCGAGGCTACTCGCACTCTGACTGAGATCCAGTCCACCGCAGACCGTCAGATC
TTCGAAGAGAAGGTCGGGCCTCTGGTGGGTCGGCTGCGCCTCACGGCTTCGCTCCGTCAAAACG
GAGCCAAGACCGCGTATCGCGTCAACCTAAAACTGGATCAGGCGGACGTCGTTGATTGCTCCAC
CAGCGTCTGCGGCGAGCTTCCGAAAGTGCGCTACACTCAGGTATGGTCGCACGACGTGACAATC
GTTGCGAATAGCACCGAGGCCTCGCGCAAATCGTTGTACGATTTGACCAAGTCCCTCGTCGCGA
CCTCGCAGGTCGAAGATCTTGTCGTCAACCTTGTGCCGCTGGGCCGTCGTGCGGACCCGCTAGC
CTCCACGCGTGATTCGACTAGTGGAGGAAGCGGAGGAGGATATCCATATGATGTTCCAGATTAT
GCTTCAGGAGGAGGCTCAGGAGGAGGAATGTCGGCTACCGCTGCCACCGTCCCGCCTGCCGCCC
CGGCCGGCGAGGGTGGCCCCCCTGCACCTCCTCCAAACCTTACTAGTAACAGGAGACTGCAGCA
GACCCAGGCCCAGGTGGATGAGGTGGTGGACATCATGAGGGTGAATGTGGACAAGGTCCTGGAG
CGGGACCAGAAGTTGTCGGAGCTGGATGACCGTGCAGATGCCCTCCAGGCAGGGGCCTCCCAGT
TTGAAACAAGTGCAGCCAAGCTCAAGCGCAAATACTGGTGGAAAAACCTCAAGATGATGATCAT
CTTGGGAGTGATCTGCGCCATCATCCTCATCATCATCATCGTTTACTTCAGCACTTGAAAGCTT
ATCGATAATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTAT
GGCTTTCATTTTCTCCTCCTTGTATAAATCCTGGTTGCTGTCTCTTTATGAGGAGTTGTGGCCC
GTTGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCGGA
ACTCATCGCCGCCTGCCTTGCCCGCTGCTGGACAGGGGCTCGGCTGTTGGGCACTGACAATTCC
GTGGTGTTGTCGGGGAAATCATCGTCCTTTCCTTGGCTGCTCGCCTATGTTGCCACCTGGATTC
TGCGCGGGACGTCCTTCTGCTACGTCCCTTCGGCCCTCAATCCAGCGGACCTTCCTTCCCGCGG
CCTGCTGCCGGCTCTGCGGCCTCTTCCGCGTCTTCGCCTTCGCCCTCAGACGAGTCGGATCTCC
CTTTGGGCCGCCTCCCCGCATCGATACCGAGCGCTGCTCGAGAGATCTACGGGTGGCATCCCTG
TGACCCCTCCCCAGTGCCTCTCCTGGCCCTGGAAGTTGCCACTCCAGTGCCCACCAGCCTTGTC
CTAATAAAATTAAGTTGCATCATTTTGTCTGACTAGGTGTCCTTCTATAATATTATGGGGTGGA
GGGGGGTGGTATGGAGCAAGGGGCAAGTTGGGAAGACAACCTGTAGGGCCTGCGGGGTCTATTG
GGAACCAAGCTGGAGTGCAGTGGCACAATCTTGGCTCACTGCAATCTCCGCCTCCTGGGTTCAA
GCGATTCTCCTGCCTCAGCCTCCCGAGTTGTTGGGATTCCAGGCATGCATGACCAGGCTCAGCT
AATTTTTGTTTTTTTGGTAGAGACGGGGTTTCACCATATTGGCCAGGCTGGTCTCCAACTCCTA
ATCTCAGGTGATCTACCCACCTTGGCCTCCCAAATTGCTGGGATTACAGGCGTGAACCACTGCT
CCCTTCCCTGTCCTTCTGATTTTGTAGGTAACCACGTGCGGACCGAGCGGCCGCAGGAACCCCT
AGTGATGGAGTTGGCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGCAGCTGCCTGC
AGGGGCGCCTGATGCGGTATTTTCTCCTTACGCATCTGTGCGGTATTTCACACCGCATACGTCA
AAGCAACCATAGTACGCGCCCTGTAGCGGCGCATTAAGCGCGGCGGGTGTGGTGGTTACGCGCA
GCGTGACCGCTACACTTGCCAGCGCCTTAGCGCCCGCTCCTTTCGCTTTCTTCCCTTCCTTTCT
CGCCACGTTCGCCGGCTTTCCCCGTCAAGCTCTAAATCGGGGGCTCCCTTTAGGGTTCCGATTT
AGTGCTTTACGGCACCTCGACCCCAAAAAACTTGATTTGGGTGATGGTTCACGTAGTGGGCCAT
CGCCCTGATAGACGGTTTTTCGCCCTTTGACGTTGGAGTCCACGTTCTTTAATAGTGGACTCTT
GTTCCAAACTGGAACAACACTCAACTCTATCTCGGGCTATTCTTTTGATTTATAAGGGATTTTG
CCGATTTCGGTCTATTGGTTAAAAAATGAGCTGATTTAACAAAAATTTAACGCGAATTTTAACA
AAATATTAACGTTTACAATTTTATGGTGCACTCTCAGTACAATCTGCTCTGATGCCGCATAGTT
AAGCCAGCCCCGACACCCGCCAACACCCGCTGACGCGCCCTGACGGGCTTGTCTGCTCCCGGCA
TCCGCTTACAGACAAGCTGTGACCGTCTCCGGGAGCTGCATGTGTCAGAGGTTTTCACCGTCAT
CACCGAAACGCGCGAGACGAAAGGGCCTCGTGATACGCCTATTTTTATAGGTTAATGTCATGAT
AATAATGGTTTCTTAGACGTCAGGTGGCACTTTTCGGGGAAATGTGCGCGGAACCCCTATTTGT
TTATTTTTCTAAATACATTCAAATATGTATCCGCTCATGAGACAATAACCCTGATAAATGCTTC
AATAATATTGAAAAAGGAAGAGTATGAGTATTCAACATTTCCGTGTCGCCCTTATTCCCTTTTT
TGCGGCATTTTGCCTTCCTGTTTTTGCTCACCCAGAAACGCTGGTGAAAGTAAAAGATGCTGAA
GATCAGTTGGGTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATGTGGCGCGGT
ATTATCCCGTATTGACGCCGGGCAAGAGCAACTCGGTCGCCGCATACACTATTCTCAGAATGAC
TTGGTTGAGTACTCACCAGTCACAGAAAAGCATCTTACGGATGGCATGACAGTAAGAGAATTAT
GCAGTGCTGCCATAACCATGAGTGATAACACTGCGGCCAACTTACTTCTGACAACGATCGGAGG
ACCGAAGGAGCTAACCGCTTTTTTGCACAACATGGGGGATCATGTAACTCGCCTTGATCGTTGG
GAACCGGAGCTGAATGAAGCCATACCAAACGACGAGCGTGACACCACGATGCCTGTAGCAATGG
CAACAACGTTGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACTGGGGC
CAGATGGTAAGCCCTCCCGTATCGTAGTTATCTACACGACGGGGAGTCAGGCAACTATGGATGA
ACGAAATAGACAGATCGCTGAGATAGGTGCCTCACTGATTAAGCATTGGTAACTGTCAGACCAA
GTTTACTCATATATACTTTAGATTGATTTAAAACTTCATTTTTAATTTAAAAGGATCTAGGTGA
AGATCCTTTTTGATAATCTCATGACCAAAATCCCTTAACGTGAGTTTTCGTTCCACTGAGCGTC
AGACCCCGTAGAAAAGATCAAAGGATCTTCTTGAGATCCTTTTTTTCTGCGCGTAATCTGCTGC
TTGCAAACAAAAAAACCACCGCTACCAGCGGTGGTTTGTTTGCCGGATCAAGAGCTACCAACTC
TTTTTCCGAAGGTAACTGGCTTCAGCAGAGCGCAGATACCAAATACTGTTCTTCTAGTGTAGCC
GTAGTTAGGCCACCACTTCAAGAACTCTGTAGCACCGCCTACATACCTCGCTCTGCTAATCCTG
TTACCAGTGGCTGCTGCCAGTGGCGATAAGTCGTGTCTTACCGGGTTGGACTCAAGACGATAGT
TACCGGATAAGGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAACGCGGCCTTT
TTACGGTTCCTGGCCTTTTGCTGGCCTTTTGCTCACATGT

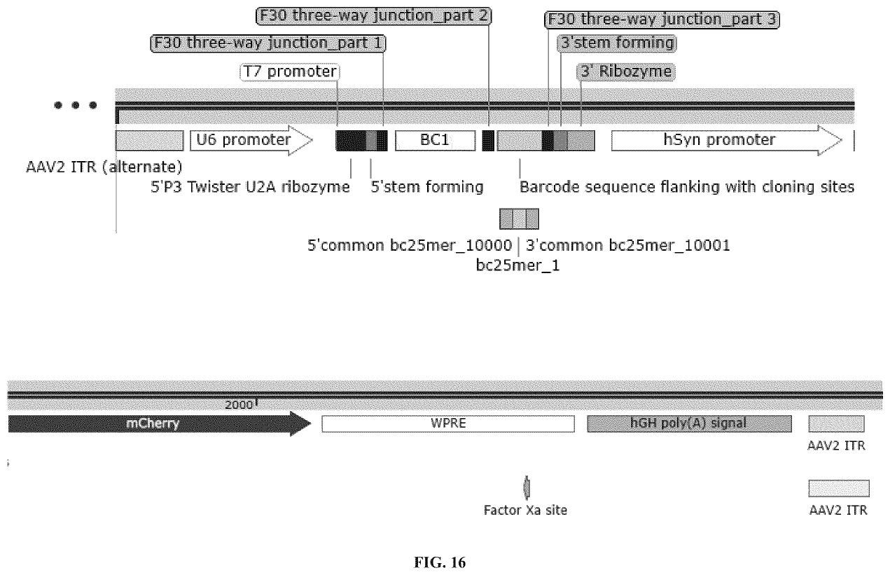

Plasmid encoding racRNA-BC1 (see FIG. 16 for a map of the plasmid)

(SEQ ID NO: 86)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTGAGGGCCTATTTCCCATGATTCCTTCATATTTGCATATACGATACAAG
GCTGTTAGAGAGATAATTGGAATTAATTTGACTGTAAACACAAAGATATTAGTACAAAATACGT
GACGTAGAAAGTAATAATTTCTTGGGTAGTTTGCAGTTTTAAAATTATGTTTTAAAATGGACTA
TCATATGCTTACCGTAACTTGAAAGTATTTCGATTTCTTGGCTTTATATATCTTGTGGAAAGGA
CGAAACACCGTGCTCGCTTCGGCAGCACATATACTAGTCGACGGGCCGCACTCGCCGGTCCCAA
GCCCGGATAAAATGGGAGGGGGGGGGAAACCGCCTAACCATGCCGAGTGCGGCCGCTTGCCATG
TGTATCGGTCCGCTTAAGAAAAAAAAAGGGGTTGGGGATTTAGCTCAGTGGTAGAGCGCTTGCC
TAGCAAGCGCAAGGCCCTGGGTTCGGTCCTCAGCTCTGGAAAAAAAAAAAAAAAAAAAAAAAGA
CAAAATAACAAAAAGACCAAAAAAAAACAAGGTAACTGGCACACACAACCTTTAAAAAAAAAGT
TAACCGGTCCGATACTCTGATGATGGGTCCCCCTAGGTTAAGGATGCACCGACGGGACGTTCTA
TGAGGACGAATCTCCCGCTTATAAGATTCTATAAACTGTGCGGTCCTTCAATTGGGGTCCCATC
ATTCATGGCAAGTGGCCGCGGTCGGCGTGGACTGTAGAACACTGCCAATGCCGGTCCCAAGCCC
GGATAAAAGTGGAGGGTACAGTCCACGCTTTTTTTCTAGACTGCAGAGGGCCCTGCGTATGAGT
GCAAGTGGGTTTTAGGACCAGGATGAGGCGGGGTGGGGGTGCCTACCTGACGACCGACCCCGAC
CCACTGGACAAGCACCCAACCCCCATTCCCCAAATTGCGCATCCCCTATCAGAGAGGGGGAGGG
GAAACAGGATGCGGCGAGGCGCGTGCGCACTGCCAGCTTCAGCACCGCGGACAGTGCCTTCGCC
CCCGCCTGGCGGCGCGCGCCACCGCCGCCTCAGCACTGAAGGCGCGCTGACGTCACTCGCCGGT
CCCCCGCAAACTCCCCTTCCCGGCCACCTTGGTCGCGTCCGCGCCGCCGCCGGCCCAGCCGGAC
CGCACCACGCGAGGCGCGAGATAGGGGGGCACGGGCGCGACCATCTGCGCTGCGGCGCCGGCGA
CTCAGCGCTGCCTCAGTCTGCGGTGGGCAGCGGAGGAGTCGTGTCGTGCCTGAGAGCGCAGTCG
AGAAGGTACCGGATCCGTGAGCAAGGGCGAGGAGGATAACATGGCCATCATCAAGGAGTTCATG
CGCTTCAAGGTGCACATGGAGGGCTCCGTGAACGGCCACGAGTTCGAGATCGAGGGCGAGGGCG
AGGGCCGCCCCTACGAGGGCACCCAGACCGCCAAGCTGAAGGTGACCAAGGGTGGCCCCCTGCC
CTTCGCCTGGGACATCCTGTCCCCTCAGTTCATGTACGGCTCCAAGGCCTACGTGAAGCACCCC
GCCGACATCCCCGACTACTTGAAGCTGTCCTTCCCCGAGGGCTTCAAGTGGGAGCGCGTGATGA
ACTTCGAGGACGGCGGCGTGGTGACCGTGACCCAGGACTCCTCCCTGCAGGACGGCGAGTTCAT
CTACAAGGTGAAGCTGCGCGGCACCAACTTCCCCTCCGACGGCCCCGTAATGCAGAAGAAGACC
ATGGGCTGGGAGGCCTCCTCCGAGCGGATGTACCCCGAGGACGGCGCCCTGAAGGGCGAGATCA
AGCAGAGGCTGAAGCTGAAGGACGGCGGCCACTACGACGCTGAGGTCAAGACCACCTACAAGGC
CAAGAAGCCCGTGCAGCTGCCCGGCGCCTACAACGTCAACATCAAGTTGGACATCACCTCCCAC
AACGAGGACTACACCATCGTGGAACAGTACGAACGCGCCGAGGGCCGCCACTCCACCGGCGGCA
TGGACGAGCTGTACAAGTAAGAATTCGATATCAAGCTTATCGATAATCAACCTCTGGATTACAA
AATTTGTGAAAGATTGACTGGTATTCTTAACTATGTTGCTCCTTTTACGCTATGTGGATACGCT
GCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTG
CACTGTGTTTGCTGACGCAACCCCCACTGGTTGGGGCATTGCCACCACCTGTCAGCTCCTTTCC
GGGACTTTCGCTTTCCCCCTCCCTATTGCCACGGCGGAACTCATCGCCGCCTGCCTTGCCCGCT
GCTGGACAGGGGCTCGGCTGTTGGGCACTGACAATTCCGTGGTGTTGTCGGGGAAATCATCGTC
CTTTCCTTGGCTGCTCGCCTATGTTGCCACCTGGATTCTGCGCGGGACGTCCTTCTGCTACGTC
CCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCATCGATA
CCGAGCGCTGCTCGAGAGATCTACGGGTGGCATCCCTGTGACCCCTCCCCAGTGCCTCTCCTGG
CCCTGGAAGTTGCCACTCCAGTGCCCACCAGCCTTGTCCTAATAAAATTAAGTTGCATCATTTT
GTCTGACTAGGTGTCCTTCTATAATATTATGGGGTGGAGGGGGGTGGTATGGAGCAAGGGGCAA
GTTGGGAAGACAACCTGTAGGGCCTGCGGGGTCTATTGGGAACCAAGCTGGAGTGCAGTGGCAC
AATCTTGGCTCACTGCAATCTCCGCCTCCTGGGTTCAAGCGATTCTCCTGCCTCAGCCTCCCGA
GTTGTTGGGATTCCAGGCATGCATGACCAGGCTCAGCTAATTTTTGTTTTTTTGGTAGAGACGG
GGTTTCACCATATTGGCCAGGCTGGTCTCCAACTCCTAATCTCAGGTGATCTACCCACCTTGGC
CTCCCAAATTGCTGGGATTACAGGCGTGAACCACTGCTCCCTTCCCTGTCCTTCTGATTTTGTA
GGTAACCACGTGCGGACCGAGCGGCCGCAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGCCTGCAGGGGCGCCTGATGCGGTATTTTCTC
CTTACGCATCTGTGCGGTATTTCACACCGCATACGTCAAAGCAACCATAGTACGCGCCCTGTAG
CGGCGCATTAAGCGCGGCGGGTGTGGTGGTTACGCGCAGCGTGACCGCTACACTTGCCAGCGCC
TTAGCGCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGACCCCAA
AAAACTTGATTTGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACT
CTATCTCGGGCTATTCTTTTGATTTATAAGGGATTTTGCCGATTTCGGTCTATTGGTTAAAAAA
TGAGCTGATTTAACAAAAATTTAACGCGAATTTTAACAAAATATTAACGTTTACAATTTTATGG
TGCACTCTCAGTACAATCTGCTCTGATGCCGCATAGTTAAGCCAGCCCCGACACCCGCCAACAC
CCGCTGACGCGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAGACGAAAGGGC
CTCGTGATACGCCTATTTTTATAGGTTAATGTCATGATAATAATGGTTTCTTAGACGTCAGGTG
GCACTTTTCGGGGAAATGTGCGCGGAACCCCTATTTGTTTATTTTTCTAAATACATTCAAATAT
GTATCCGCTCATGAGACAATAACCCTGATAAATGCTTCAATAATATTGAAAAAGGAAGAGTATG
AGTATTCAACATTTCCGTGTCGCCCTTATTCCCTTTTTTGCGGCATTTTGCCTTCCTGTTTTTG
CTCACCCAGAAACGCTGGTGAAAGTAAAAGATGCTGAAGATCAGTTGGGTGCACGAGTGGGTTA
CATCGAACTGGATCTCAACAGCGGTAAGATCCTTGAGAGTTTTCGCCCCGAAGAACGTTTTCCA
ATGATGAGCACTTTTAAAGTTCTGCTATGTGGCGCGGTATTATCCCGTATTGACGCCGGGCAAG
AGCAACTCGGTCGCCGCATACACTATTCTCAGAATGACTTGGTTGAGTACTCACCAGTCACAGA
AAAGCATCTTACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACCGCTTTTTTGC
ACAACATGGGGGATCATGTAACTCGCCTTGATCGTTGGGAACCGGAGCTGAATGAAGCCATACC
AAACGACGAGCGTGACACCACGATGCCTGTAGCAATGGCAACAACGTTGCGCAAACTATTAACT
GGCGAACTACTTACTCTAGCTTCCCGGCAACAATTAATAGACTGGATGGAGGCGGATAAAGTTG
CAGGACCACTTCTGCGCTCGGCCCTTCCGGCTGGCTGGTTTATTGCTGATAAATCTGGAGCCGG
TGAGCGTGGGTCTCGCGGTATCATTGCAGCACTGGGGCCAGATGGTAAGCCCTCCCGTATCGTA
GTTATCTACACGACGGGGAGTCAGGCAACTATGGATGAACGAAATAGACAGATCGCTGAGATAG
GTGCCTCACTGATTAAGCATTGGTAACTGTCAGACCAAGTTTACTCATATATACTTTAGATTGA
TTTAAAACTTCATTTTTAATTTAAAAGGATCTAGGTGAAGATCCTTTTTGATAATCTCATGACC
AAAATCCCTTAACGTGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAAAAACCACCGCTACC
AGCGGTGGTTTGTTTGCCGGATCAAGAGCTACCAACTCTTTTTCCGAAGGTAACTGGCTTCAGC
AGAGCGCAGATACCAAATACTGTTCTTCTAGTGTAGCCGTAGTTAGGCCACCACTTCAAGAACT
CTGTAGCACCGCCTACATACCTCGCTCTGCTAATCCTGTTACCAGTGGCTGCTGCCAGTGGCGA
TAAGTCGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAGATACC
TACAGCGTGAGCTATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAACGCCTGGTATCTT
TATAGTCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGCTGGCC
TTTTGCTCACATGT.

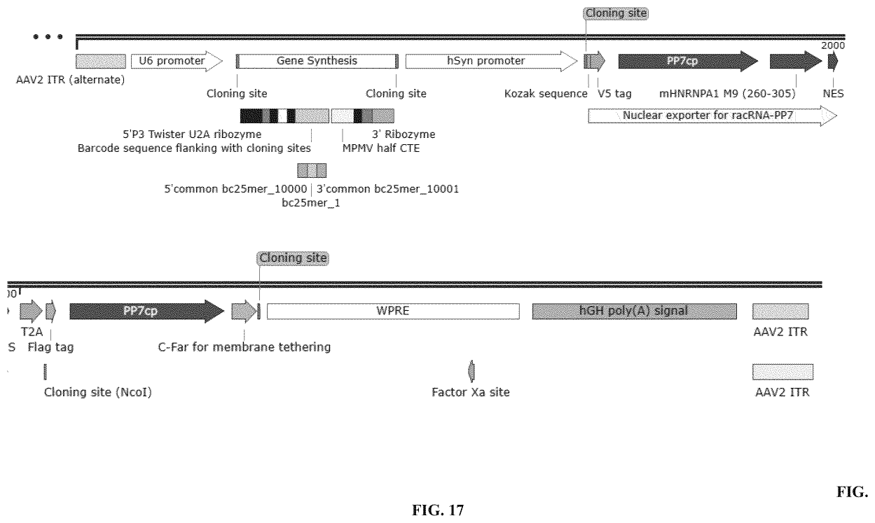

Plasmid encoding racRNA-hCTE-PP7 (see FIG. 17 for a map of the plasmid)

(SEQ ID NO: 87)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTGAGGGCCTATTTCCCATGATTCCTTCATATTTGCATATACGATACAAG
GCTGTTAGAGAGATAATTGGAATTAATTTGACTGTAAACACAAAGATATTAGTACAAAATACGT
GACGTAGAAAGTAATAATTTCTTGGGTAGTTTGCAGTTTTAAAATTATGTTTTAAAATGGACTA
TCATATGCTTACCGTAACTTGAAAGTATTTCGATTTCTTGGCTTTATATATCTTGTGGAAAGGA
CGAAACACCGTGCTCGCTTCGGCAGCACATATACTAGTCGACGGGCCGCACTCGCCGGTCCCAA
GCCCGGATAAAATGGGAGGGGGGGGGAAACCGCCTAACCATGCCGAGTGCGGCCGCTTGCCATG
TGTATCGGTCCGGGAGCAGACGATATGGCGTCGCTCCCGGTCCGATACTCTGATGATCCTAGGT
TAAGGATGCACCGACGGGACGTTCTATGAGGACGAATCTCCCGCTTATAAGATTCTATAAACTG
TGCGGTCCTTCAATTGGGGTCCCCACTAACCTAAGACAGGAGGGCCGGGAAACCTGCCTAATCC
AATGACGGGTAATAGTGGGGACCCATCATTCATGGCAAGTGGCCGCGGTCGGCGTGGACTGTAG
AACACTGCCAATGCCGGTCCCAAGCCCGGATAAAAGTGGAGGGTACAGTCCACGCTTTTTTTCT
AGACTGCAGAGGGCCCTGCGTATGAGTGCAAGTGGGTTTTAGGACCAGGATGAGGCGGGGTGGG
GGTGCCTACCTGACGACCGACCCCGACCCACTGGACAAGCACCCAACCCCCATTCCCCAAATTG
CGCATCCCCTATCAGAGAGGGGGAGGGGAAACAGGATGCGGCGAGGCGCGTGCGCACTGCCAGC
TTCAGCACCGCGGACAGTGCCTTCGCCCCCGCCTGGCGGCGCGCGCCACCGCCGCCTCAGCACT
GAAGGCGCGCTGACGTCACTCGCCGGTCCCCCGCAAACTCCCCTTCCCGGCCACCTTGGTCGCG
TCCGCGCCGCCGCCGGCCCAGCCGGACCGCACCACGCGAGGCGCGAGATAGGGGGGCACGGGCG
CGACCATCTGCGCTGCGGCGCCGGCGACTCAGCGCTGCCTCAGTCTGCGGTGGGCAGCGGAGGA
GTCGTGTCGTGCCTGAGAGCGCAGTCGAGAAGGTACCGGATCCGCCACCATGGGCAAGCCCATC
CCCAACCCCCTGCTGGGCCTGGACAGCACCGGCGGTGGAGGTTCCTCCAAAACCATCGTTCTTT
CGGTCGGCGAGGCTACTCGCACTCTGACTGAGATCCAGTCCACCGCAGACCGTCAGATCTTCGA
AGAGAAGGTCGGGCCTCTGGTGGGTCGGCTGCGCCTCACGGCTTCGCTCCGTCAAAACGGAGCC
AAGACCGCGTATCGCGTCAACCTAAAACTGGATCAGGCGGACGTCGTTGATTGCTCCACCAGCG
TCTGCGGCGAGCTTCCGAAAGTGCGCTACACTCAGGTATGGTCGCACGACGTGACAATCGTTGC
GAATAGCACCGAGGCCTCGCGCAAATCGTTGTACGATTTGACCAAGTCCCTCGTCGCGACCTCG
CAGGTCGAAGATCTTGTCGTCAACCTTGTGCCGCTGGGCCGTGGCGGTGGCGGATCTGGCGGCG
GTGGTAGCAATGATTTTGGCAATTACAACAATCAGTCTTCCAATTTTGGGCCGATGAAGGGAGG
AAACTTTGGAGGCAGGAGCTCTGGCCCTTATGGTGGTGGAGGCCAGTACTTTGCTAAACCACGG
AACCAAGGTGGCTATGGCGGAGGTGGTTCTCTGCCTCCACTTGAAAGACTGACACTGGGCTCAG
GAGGATCTGGTGGTTCTGAGGGCAGAGGAAGTCTTCTAACATGCGGTGACGTGGAGGAGAATCC
CGGCCCTGCCATGGATTACAAGGATGACGACGATAAGGGCGGAGGTGGTTCTTCCAAAACCATC
GTTCTTTCGGTCGGCGAGGCTACTCGCACTCTGACTGAGATCCAGTCCACCGCAGACCGTCAGA
TCTTCGAAGAGAAGGTCGGGCCTCTGGTGGGTCGGCTGCGCCTCACGGCTTCGCTCCGTCAAAA
CGGAGCCAAGACCGCGTATCGCGTCAACCTAAAACTGGATCAGGCGGACGTCGTTGATTGCTCC
ACCAGCGTCTGCGGCGAGCTTCCGAAAGTGCGCTACACTCAGGTATGGTCGCACGACGTGACAA
TCGTTGCGAATAGCACCGAGGCCTCGCGCAAATCGTTGTACGATTTGACCAAGTCCCTCGTCGC
GACCTCGCAGGTCGAAGATCTTGTCGTCAACCTTGTGCCGCTGGGCCGTGGCGGAGGTGGTTCT
AAGCTGAACCCTCCTGATGAGAGTGGCCCCGGCTGCATGAGCTGCTGTGTGCTCTCCTAAGAAT
TCGATATCAAGCTTATCGATAATCAACCTCTGGATTACAAAATTTGTGAAAGATTGACTGGTAT
TCTTAACTATGTTGCTCCTTTTACGCTATGTGGATACGCTGCTTTAATGCCTTTGTATCATGCT
ATTGCTTCCCGTATGGCTTTCATTTTCTCCTCCTTGTATAAATCCTGGTTGCTGTCTCTTTATG
AGGAGTTGTGGCCCGTTGTCAGGCAACGTGGCGTGGTGTGCACTGTGTTTGCTGACGCAACCCC
CACTGGTTGGGGCATTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGGGGCTCGGCTGTTGG
GCACTGACAATTCCGTGGTGTTGTCGGGGAAATCATCGTCCTTTCCTTGGCTGCTCGCCTATGT
TGCCACCTGGATTCTGCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTCGCCTTCGCCCTCAGA
CGAGTCGGATCTCCCTTTGGGCCGCCTCCCCGCATCGATACCGAGCGCTGCTCGAGAGATCTAC
GGGTGGCATCCCTGTGACCCCTCCCCAGTGCCTCTCCTGGCCCTGGAAGTTGCCACTCCAGTGC
CCACCAGCCTTGTCCTAATAAAATTAAGTTGCATCATTTTGTCTGACTAGGTGTCCTTCTATAA
TATTATGGGGTGGAGGGGGGTGGTATGGAGCAAGGGGCAAGTTGGGAAGACAACCTGTAGGGCC
TGCGGGGTCTATTGGGAACCAAGCTGGAGTGCAGTGGCACAATCTTGGCTCACTGCAATCTCCG
CCTCCTGGGTTCAAGCGATTCTCCTGCCTCAGCCTCCCGAGTTGTTGGGATTCCAGGCATGCAT
GACCAGGCTCAGCTAATTTTTGTTTTTTTGGTAGAGACGGGGTTTCACCATATTGGCCAGGCTG
GTCTCCAACTCCTAATCTCAGGTGATCTACCCACCTTGGCCTCCCAAATTGCTGGGATTACAGG
CGTGAACCACTGCTCCCTTCCCTGTCCTTCTGATTTTGTAGGTAACCACGTGCGGACCGAGCGG
CCGCAGGAACCCCTAGTGATGGAGTTGGCCACTCCCTCTCTGCGCGCTCGCTCGCTCACTGAGG
CCGGGCGACCAAAGGTCGCCCGACGCCCGGGCTTTGCCCGGGGGGCCTCAGTGAGCGAGCGAGC
GCGCAGCTGCCTGCAGGGGCGCCTGATGCGGTATTTTCTCCTTACGCATCTGTGCGGTATTTCA
CACCGCATACGTCAAAGCAACCATAGTACGCGCCCTGTAGCGGCGCATTAAGCGCGGCGGGTGT
GGTGGTTACGCGCAGCGTGACCGCTACACTTGCCAGCGCCTTAGCGCCCGCTCCTTTCGCTTTC
TTCCCTTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTTGGGTGATGGTTC
ACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACTCTATCTCGGGCTATTCTTTTGATT
TATAAGGGATTTTGCCGATTTCGGTCTATTGGTTAAAAAATGAGCTGATTTAACAAAAATTTAA
CGCGAATTTTAACAAAATATTAACGTTTACAATTTTATGGTGCACTCTCAGTACAATCTGCTCT
GATGCCGCATAGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCATGTGTCAGAG
GTTTTCACCGTCATCACCGAAACGCGCGAGACGAAAGGGCCTCGTGATACGCCTATTTTTATAG
GTTAATGTCATGATAATAATGGTTTCTTAGACGTCAGGTGGCACTTTTCGGGGAAATGTGCGCG
GAACCCCTATTTGTTTATTTTTCTAAATACATTCAAATATGTATCCGCTCATGAGACAATAACC
CTGATAAATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAACAGCGG
TAAGATCCTTGAGAGTTTTCGCCCCGAAGAACGTTTTCCAATGATGAGCACTTTTAAAGTTCTG
CTATGTGGCGCGGTATTATCCCGTATTGACGCCGGGCAAGAGCAACTCGGTCGCCGCATACACT
ATTCTCAGAATGACTTGGTTGAGTACTCACCAGTCACAGAAAAGCATCTTACGGATGGCATGAC
AGTAAGAGAATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGACACCACGAT
GCCTGTAGCAATGGCAACAACGTTGCGCAAACTATTAACTGGCGAACTACTTACTCTAGCTTCC
CGGCAACAATTAATAGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGGTCTCGCGGTATCAT
TGCAGCACTGGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAAGCATTGGT
AACTGTCAGACCAAGTTTACTCATATATACTTTAGATTGATTTAAAACTTCATTTTTAATTTAA
AAGGATCTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCA
AGAGCTACCAACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTACATACCTCG
CTCTGCTAATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTGTCGGGTTTCGCCAC
CTCTGACTTGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGT.

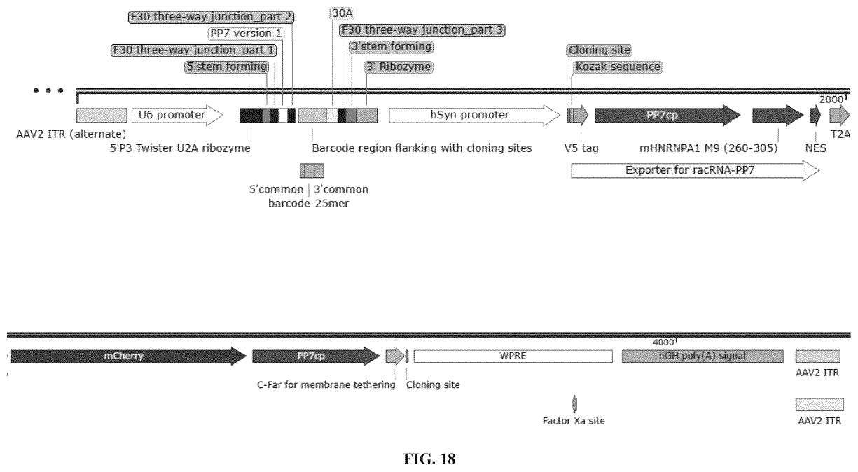

Plasmid encoding racRNA-30A-exporter-mCherry (see FIG. 18 for a map of the plasmid)

(SEQ ID NO: 88)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCACGCGTGAGGGCCTATTTCCCATGATTCCTTCATATTTGCATATACGATACAAG
GCTGTTAGAGAGATAATTGGAATTAATTTGACTGTAAACACAAAGATATTAGTACAAAATACGT
GACGTAGAAAGTAATAATTTCTTGGGTAGTTTGCAGTTTTAAAATTATGTTTTAAAATGGACTA
TCATATGCTTACCGTAACTTGAAAGTATTTCGATTTCTTGGCTTTATATATCTTGTGGAAAGGA
CGAAACACCGTGCTCGCTTCGGCAGCACATATACTAGTCGACGGGCCGCACTCGCCGGTCCCAA
GCCCGGATAAAATGGGAGGGGGGGGGAAACCGCCTAACCATGCCGAGTGCGGCCGCTTGCCATG
TGTATCGGTCCGGGAGCAGACGATATGGCGTCGCTCCCGGTCCGATACTCTGATGATGGGTCCC
CCTAGGGACGGGACGTTCTGCTAAGATCATTTCTCCCTGGGGCAGATTCTATAAACTGTGCGGT
CCTTCAATTGAAAAAAAAAAAAAAAAAAAAAAAAAAAAAAGGGTCCCATCATTCATGGCAAGTG
GCCGCGGTCGGCGTGGACTGTAGAACACTGCCAATGCCGGTCCCAAGCCCGGATAAAAGTGGAG
GGTACAGTCCACGCTTTTTTTCTAGACTGCAGAGGGCCCTGCGTATGAGTGCAAGTGGGTTTTA
GGACCAGGATGAGGCGGGGTGGGGGTGCCTACCTGACGACCGACCCCGACCCACTGGACAAGCA
CCCAACCCCCATTCCCCAAATTGCGCATCCCCTATCAGAGAGGGGGAGGGGAAACAGGATGCGG
CGAGGCGCGTGCGCACTGCCAGCTTCAGCACCGCGGACAGTGCCTTCGCCCCCGCCTGGCGGCG
CGCGCCACCGCCGCCTCAGCACTGAAGGCGCGCTGACGTCACTCGCCGGTCCCCCGCAAACTCC
CCTTCCCGGCCACCTTGGTCGCGTCCGCGCCGCCGCCGGCCCAGCCGGACCGCACCACGCGAGG
CGCGAGATAGGGGGGCACGGGCGCGACCATCTGCGCTGCGGCGCCGGCGACTCAGCGCTGCCTC
AGTCTGCGGTGGGCAGCGGAGGAGTCGTGTCGTGCCTGAGAGCGCAGTCGAGAAGGTACCGGAT
CCGCCACCATGGGCAAGCCCATCCCCAACCCCCTGCTGGGCCTGGACAGCACCGGCGGTGGAGG
TTCCTCCAAAACCATCGTTCTTTCGGTCGGCGAGGCTACTCGCACTCTGACTGAGATCCAGTCC
ACCGCAGACCGTCAGATCTTCGAAGAGAAGGTCGGGCCTCTGGTGGGTCGGCTGCGCCTCACGG
CTTCGCTCCGTCAAAACGGAGCCAAGACCGCGTATCGCGTCAACCTAAAACTGGATCAGGCGGA
CGTCGTTGATTGCTCCACCAGCGTCTGCGGCGAGCTTCCGAAAGTGCGCTACACTCAGGTATGG
TCGCACGACGTGACAATCGTTGCGAATAGCACCGAGGCCTCGCGCAAATCGTTGTACGATTTGA
CCAAGTCCCTCGTCGCGACCTCGCAGGTCGAAGATCTTGTCGTCAACCTTGTGCCGCTGGGCCG
TGGCGGTGGCGGATCTGGCGGCGGTGGTAGCAATGATTTTGGCAATTACAACAATCAGTCTTCC
AATTTTGGGCCGATGAAGGGAGGAAACTTTGGAGGCAGGAGCTCTGGCCCTTATGGTGGTGGAG
GCCAGTACTTTGCTAAACCACGGAACCAAGGTGGCTATGGCGGAGGTGGTTCTCTGCCTCCACT
TGAAAGACTGACACTGGGCTCAGGAGGATCTGGTGGTTCTGAGGGCAGAGGAAGTCTTCTAACA
TGCGGTGACGTGGAGGAGAATCCCGGCCCTGCCATGGTGAGCAAGGGCGAGGAGGATAACATGG
CCATCATCAAGGAGTTCATGCGCTTCAAGGTGCACATGGAGGGCTCCGTGAACGGCCACGAGTT
CGAGATCGAGGGCGAGGGCGAGGGCCGCCCCTACGAGGGCACCCAGACCGCCAAGCTGAAGGTG
ACCAAGGGTGGCCCCCTGCCCTTCGCCTGGGACATCCTGTCCCCTCAGTTCATGTACGGCTCCA
AGGCCTACGTGAAGCACCCCGCCGACATCCCCGACTACTTGAAGCTGTCCTTCCCCGAGGGCTT
CAAGTGGGAGCGCGTGATGAACTTCGAGGACGGCGGCGTGGTGACCGTGACCCAGGACTCCTCC
CTGCAGGACGGCGAGTTCATCTACAAGGTGAAGCTGCGCGGCACCAACTTCCCCTCCGACGGCC
CCGTAATGCAGAAGAAGACCATGGGCTGGGAGGCCTCCTCCGAGCGGATGTACCCCGAGGACGG
CGCCCTGAAGGGCGAGATCAAGCAGAGGCTGAAGCTGAAGGACGGCGGCCACTACGACGCTGAG
GTCAAGACCACCTACAAGGCCAAGAAGCCCGTGCAGCTGCCCGGCGCCTACAACGTCAACATCA
AGTTGGACATCACCTCCCACAACGAGGACTACACCATCGTGGAACAGTACGAACGCGCCGAGGG
CCGCCACTCCACCGGCGGCATGGACGAGCTGTACAAGGGCGGAGGTGGTTCTTCCAAAACCATC
GTTCTTTCGGTCGGCGAGGCTACTCGCACTCTGACTGAGATCCAGTCCACCGCAGACCGTCAGA
TCTTCGAAGAGAAGGTCGGGCCTCTGGTGGGTCGGCTGCGCCTCACGGCTTCGCTCCGTCAAAA
CGGAGCCAAGACCGCGTATCGCGTCAACCTAAAACTGGATCAGGCGGACGTCGTTGATTGCTCC
ACCAGCGTCTGCGGCGAGCTTCCGAAAGTGCGCTACACTCAGGTATGGTCGCACGACGTGACAA
TCGTTGCGAATAGCACCGAGGCCTCGCGCAAATCGTTGTACGATTTGACCAAGTCCCTCGTCGC
GACCTCGCAGGTCGAAGATCTTGTCGTCAACCTTGTGCCGCTGGGCCGTGGCGGAGGTGGTTCT
AAGCTGAACCCTCCTGATGAGAGTGGCCCCGGCTGCATGAGCTGCTGTGTGCTCTCCTAAGAAT
TCGATATCAAGCTTATCGATAATCAACCTCTGGATTACAAAATTTGTGAAAGATTGACTGGTAT
TCTTAACTATGTTGCTCCTTTTACGCTATGTGGATACGCTGCTTTAATGCCTTTGTATCATGCT
ATTGCTTCCCGTATGGCTTTCATTTTCTCCTCCTTGTATAAATCCTGGTTGCTGTCTCTTTATG
AGGAGTTGTGGCCCGTTGTCAGGCAACGTGGCGTGGTGTGCACTGTGTTTGCTGACGCAACCCC
CACTGGTTGGGGCATTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGGGGCTCGGCTGTTGG
GCACTGACAATTCCGTGGTGTTGTCGGGGAAATCATCGTCCTTTCCTTGGCTGCTCGCCTATGT
TGCCACCTGGATTCTGCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTCGCCTTCGCCCTCAGA
CGAGTCGGATCTCCCTTTGGGCCGCCTCCCCGCATCGATACCGAGCGCTGCTCGAGAGATCTAC
GGGTGGCATCCCTGTGACCCCTCCCCAGTGCCTCTCCTGGCCCTGGAAGTTGCCACTCCAGTGC
CCACCAGCCTTGTCCTAATAAAATTAAGTTGCATCATTTTGTCTGACTAGGTGTCCTTCTATAA
TATTATGGGGTGGAGGGGGGTGGTATGGAGCAAGGGGCAAGTTGGGAAGACAACCTGTAGGGCC
TGCGGGGTCTATTGGGAACCAAGCTGGAGTGCAGTGGCACAATCTTGGCTCACTGCAATCTCCG
CCTCCTGGGTTCAAGCGATTCTCCTGCCTCAGCCTCCCGAGTTGTTGGGATTCCAGGCATGCAT
GACCAGGCTCAGCTAATTTTTGTTTTTTTGGTAGAGACGGGGTTTCACCATATTGGCCAGGCTG
GTCTCCAACTCCTAATCTCAGGTGATCTACCCACCTTGGCCTCCCAAATTGCTGGGATTACAGG
CGTGAACCACTGCTCCCTTCCCTGTCCTTCTGATTTTGTAGGTAACCACGTGCGGACCGAGCGG
CCGCAGGAACCCCTAGTGATGGAGTTGGCCACTCCCTCTCTGCGCGCTCGCTCGCTCACTGAGG
CCGGGCGACCAAAGGTCGCCCGACGCCCGGGCTTTGCCCGGGCGGCCTCAGTGAGCGAGCGAGC
GCGCAGCTGCCTGCAGGGGCGCCTGATGCGGTATTTTCTCCTTACGCATCTGTGCGGTATTTCA
CACCGCATACGTCAAAGCAACCATAGTACGCGCCCTGTAGCGGCGCATTAAGCGCGGCGGGTGT
GGTGGTTACGCGCAGCGTGACCGCTACACTTGCCAGCGCCTTAGCGCCCGCTCCTTTCGCTTTC
TTCCCTTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTTGGGTGATGGTTC
ACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACTCTATCTCGGGCTATTCTTTTGATT
TATAAGGGATTTTGCCGATTTCGGTCTATTGGTTAAAAAATGAGCTGATTTAACAAAAATTTAA
CGCGAATTTTAACAAAATATTAACGTTTACAATTTTATGGTGCACTCTCAGTACAATCTGCTCT
GATGCCGCATAGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCATGTGTCAGAG
GTTTTCACCGTCATCACCGAAACGCGCGAGACGAAAGGGCCTCGTGATACGCCTATTTTTATAG
GTTAATGTCATGATAATAATGGTTTCTTAGACGTCAGGTGGCACTTTTCGGGGAAATGTGCGCG
GAACCCCTATTTGTTTATTTTTCTAAATACATTCAAATATGTATCCGCTCATGAGACAATAACC
CTGATAAATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAACAGCGG
TAAGATCCTTGAGAGTTTTCGCCCCGAAGAACGTTTTCCAATGATGAGCACTTTTAAAGTTCTG
CTATGTGGCGCGGTATTATCCCGTATTGACGCCGGGCAAGAGCAACTCGGTCGCCGCATACACT
ATTCTCAGAATGACTTGGTTGAGTACTCACCAGTCACAGAAAAGCATCTTACGGATGGCATGAC
AGTAAGAGAATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGACACCACGAT
GCCTGTAGCAATGGCAACAACGTTGCGCAAACTATTAACTGGCGAACTACTTACTCTAGCTTCC
CGGCAACAATTAATAGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGGTCTCGCGGTATCAT
TGCAGCACTGGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAAGCATTGGT
AACTGTCAGACCAAGTTTACTCATATATACTTTAGATTGATTTAAAACTTCATTTTTAATTTAA
AAGGATCTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCA
AGAGCTACCAACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTACATACCTCG
CTCTGCTAATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTGTCGGGTTTCGCCAC
CTCTGACTTGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGT.

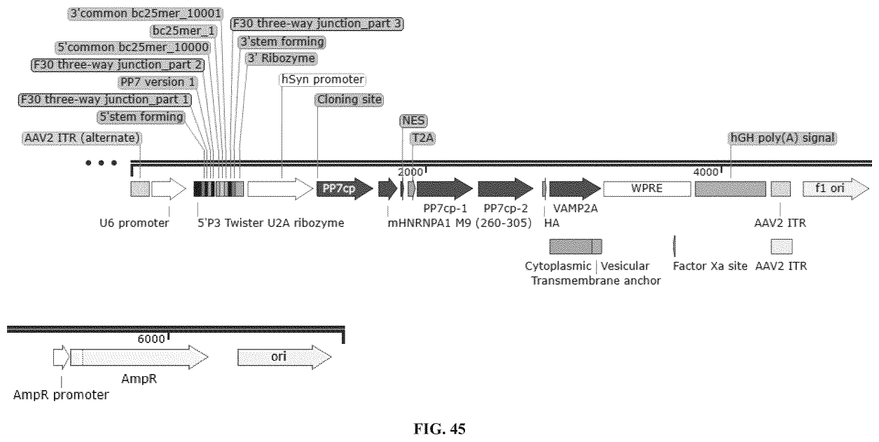

Plasmid encoding GB_M9 (see FIG. 9A) (see FIG. 45 for a map of the plasmid)

(SEQ ID NO: 85)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggaggggcggggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggccctgcgtatgagtgcaagtgggttttaggaccaggatgaggcgg
ggtgggggtgcctacctgacgaccgaccccgacccactggacaagcacccaacccccattcccc
aaattgcgcatcccctatcagagagggggaggggaaacaggatgcggcgaggcgcgtgcgcact
gccagcttcagcaccgcggacagtgccttcgcccccgcctggcggcgcgcgccaccgccgcctc
agcactgaaggcgcgctgacgtcactcgccggtcccccgcaaactccccttcccggccaccttg
gtcgcgtccgcgccgccgccggcccagccggaccgcaccacgcgaggcgcgagataggggggca
cgggcgcgaccatctgcgctgcggcgccggcgactcagcgctgcctcagtctgcggtgggcagc
ggaggagtcgtgtcgtgcctgagagcgcagtcgagaaggtaccGGATCCatgtccaaaaccatc
gttctttcggtcggcgaggctactcgcactctgactgagatccagtccaccgcagaccgtcaga
tcttcgaagagaaggtcgggcctctggtgggtcggctgcgcctcacggcttcgctccgtcaaaa
cggagccaagaccgcgtatcgcgtcaacctaaaactggatcaggcggacgtcgttgattgctcc
accagcgtctgcggcgagcttccgaaagtgcgctacactcaggtatggtcgcacgacgtgacaa
tcgttgcgaatagcaccgaggcctcgcgcaaatcgttgtacgatttgaccaagtccctcgtcgc
gacctcgcaggtcgaagatcttgtcgtcaaccttgtgccgctgggccgtggcggtggcggatct
ggcggcggtggtagcAATGATTTTGGCAATTACAACAATCAGTCTTCCAATTTTGGGCCGATGA
AGGGAGGAAACTTTGGAGGCAGGAGCTCTGGCCCTTATGGTGGTGGAGGCCAGTACTTTGCTAA
ACCACGGAACCAAGGTGGCTATGGCGGAGGTGGTTCTctgcctccacttgaaagactgacactg
ggctcaggaggatctggtggttctgagggcagaggaagtcttctaacatgcggtgacgtggagg
agaatcccggccctgccaccATGTCCAAAACCATCGTTCTTTCGGTCGGCGAGGCTACTCGCAC
TCTGACTGAGATCCAGTCCACCGCAGACCGTCAGATCTTCGAAGAGAAGGTCGGGCCTCTGGTG
GGTCGGCTGCGCCTCACGGCTTCGCTCCGTCAAAACGGAGCCAAGACCGCGTATCGCGTCAACC
TAAAACTGGATCAGGCGGACGTCGTTGATTGCTCCACCAGCGTCTGCGGCGAGCTTCCGAAAGT
GCGCTACACTCAGGTATGGTCGCACGACGTGACAATCGTTGCGAATAGCACCGAGGCCTCGCGC
AAATCGTTGTACGATTTGACCAAGTCCCTCGTCGCGACCTCGCAGGTCGAAGATCTTGTCGTCA
ACCTTGTGCCGCTGGGCCGTCGTGCGGACCCGCTAGCCTCCTgcggccgcTCCAAAACCATCGT
TCTTTCGGTCGGCGAGGCTACTCGCACTCTGACTGAGATCCAGTCCACCGCAGACCGTCAGATC
TTCGAAGAGAAGGTCGGGCCTCTGGTGGGTCGGCTGCGCCTCACGGCTTCGCTCCGTCAAAACG
GAGCCAAGACCGCGTATCGCGTCAACCTAAAACTGGATCAGGCGGACGTCGTTGATTGCTCCAC
CAGCGTCTGCGGCGAGCTTCCGAAAGTGCGCTACACTCAGGTATGGTCGCACGACGTGACAATC
GTTGCGAATAGCACCGAGGCCTCGCGCAAATCGTTGTACGATTTGACCAAGTCCCTCGTCGCGA
CCTCGCAGGTCGAAGATCTTGTCGTCAACCTTGTGCCGCTGGGCCGTCGTGCGGACCCGCTAGC
CTCCACGCGTGATTCGACTAGTGGAGGAAGCGGAGGAGGATATCCATATGATGTTCCAGATTAT
GCTtcaggaggaggctcaggaggaggaatgtcggctaccgctgccaccgtcccgcctgccgccc
cggccggcgagggtggcccccctgcacctcctccaaaccttactagtaacaggagactgcagca
gacccaggcccaggtggatgaggtggtggacatcatgagggtgaatgtggacaaggtcctggag
cgggaccagaagttgtcggagctggatgaccgtgcagatgccctccaggcaggggcctcccagt
ttgaaacaagtgcagccaagctcaagcgcaaatactggtggaaaaacctcaagatgatgatcat
cttgggagtgatctgcgccatcatcctcatcatcatcatcgtttacttcagcacttgaaagctt
atcgataatcaacctctggattacaaaatttgtgaaagattgactggtattcttaactatgttg
ctccttttacgctatgtggatacgctgctttaatgcctttgtatcatgctattgcttcccgtat
ggctttcattttctcctccttgtataaatcctggttgctgtctctttatgaggagttgtggccc
gttgtcaggcaacgtggcgtggtgtgcactgtgtttgctgacgcaacccccactggttggggca
ttgccaccacctgtcagctcctttccgggactttcgctttccccctccctattgccacggcgga
actcatcgccgcctgccttgcccgctgctggacaggggctcggctgttgggcactgacaattcc
gtggtgttgtcggggaaatcatcgtcctttccttggctgctcgcctatgttgccacctggattc
tgcgcgggacgtccttctgctacgtcccttcggccctcaatccagcggaccttccttcccgcgg
cctgctgccggctctgcggcctcttccgcgtcttcgccttcgccctcagacgagtcggatctcc
ctttgggccgcctccccgcatcgataccgagcgctgctcgagagatctacgggtggcatccctg
tgacccctccccagtgcctctcctggccctggaagttgccactccagtgcccaccagccttgtc
ctaataaaattaagttgcatcattttgtctgactaggtgtccttctataatattatggggtgga
ggggggtggtatggagcaaggggcaagttgggaagacaacctgtagggcctgcggggtctattg
ggaaccaagctggagtgcagtggcacaatcttggctcactgcaatctccgcctcctgggttcaa
gcgattctcctgcctcagcctcccgagttgttgggattccaggcatgcatgaccaggctcagct
aatttttgtttttttggtagagacggggtttcaccatattggccaggctggtctccaactccta
atctcaggtgatctacccaccttggcctcccaaattgctgggattacaggcgtgaaccactgct
cccttccctgtccttctgattttgtaggtaaccacgtgcggaccgagcggccgcaggaacccct
agtgatggagttggccactccctctctgcgcgctcgctcgctcactgaggccgggcgaccaaag
gtcgcccgacgcccgggctttgcccgggcggcctcagtgagcgagcgagcgcgcagctgcctgc
aggggcgcctgatgcggtattttctccttacgcatctgtgcggtatttcacaccgcatacgtca
aagcaaccatagtacgcgccctgtagcggcgcattaagcgcggcgggtgtggtggttacgcgca
gcgtgaccgctacacttgccagcgccttagcgcccgctcctttcgctttcttcccttcctttct
cgccacgttcgccggctttccccgtcaagctctaaatcgggggctccctttagggttccgattt
agtgctttacggcacctcgaccccaaaaaacttgatttgggtgatggttcacgtagtgggccat
cgccctgatagacggtttttcgccctttgacgttggagtccacgttctttaatagtggactctt
gttccaaactggaacaacactcaactctatctcgggctattcttttgatttataagggattttg
ccgatttcggtctattggttaaaaaatgagctgatttaacaaaaatttaacgcgaattttaaca
aaatattaacgtttacaattttatggtgcactctcagtacaatctgctctgatgccgcatagtt
aagccagccccgacacccgccaacacccgctgacgcgccctgacgggcttgtctgctcccggca
tccgcttacagacaagctgtgaccgtctccgggagctgcatgtgtcagaggttttcaccgtcat
caccgaaacgcgcgagacgaaagggcctcgtgatacgcctatttttataggttaatgtcatgat
aataatggtttcttagacgtcaggtggcacttttcggggaaatgtgcgcggaacccctatttgt
ttatttttctaaatacattcaaatatgtatccgctcatgagacaataaccctgataaatgcttc
aataatattgaaaaaggaagagtatgagtattcaacatttccgtgtcgcccttattcccttttt
tgcggcattttgccttcctgtttttgctcacccagaaacgctggtgaaagtaaaagatgctgaa
gatcagttgggtgcacgagtgggttacatcgaactggatctcaacagcggtaagatccttgaga
gttttcgccccgaagaacgttttccaatgatgagcacttttaaagttctgctatgtggcgcggt
attatcccgtattgacgccgggcaagagcaactcggtcgccgcatacactattctcagaatgac
ttggttgagtactcaccagtcacagaaaagcatcttacggatggcatgacagtaagagaattat
gcagtgctgccataaccatgagtgataacactgcggccaacttacttctgacaacgatcggagg
accgaaggagctaaccgcttttttgcacaacatgggggatcatgtaactcgccttgatcgttgg
gaaccggagctgaatgaagccataccaaacgacgagcgtgacaccacgatgcctgtagcaatgg
caacaacgttgcgcaaactattaactggcgaactacttactctagcttcccggcaacaattaat
agactggatggaggcggataaagttgcaggaccacttctgcgctcggcccttccggctggctgg
tttattgctgataaatctggagccggtgagcgtgggtctcgcggtatcattgcagcactggggc
cagatggtaagccctcccgtatcgtagttatctacacgacggggagtcaggcaactatggatga
acgaaatagacagatcgctgagataggtgcctcactgattaagcattggtaactgtcagaccaa
gtttactcatatatactttagattgatttaaaacttcatttttaatttaaaaggatctaggtga
agatcctttttgataatctcatgaccaaaatcccttaacgtgagttttcgttccactgagcgtc
agaccccgtagaaaagatcaaaggatcttcttgagatcctttttttctgcgcgtaatctgctgc
ttgcaaacaaaaaaaccaccgctaccagcggtggtttgtttgccggatcaagagctaccaactc
tttttccgaaggtaactggcttcagcagagcgcagataccaaatactgttcttctagtgtagcc
gtagttaggccaccacttcaagaactctgtagcaccgcctacatacctcgctctgctaatcctg
ttaccagtggctgctgccagtggcgataagtcgtgtcttaccgggttggactcaagacgatagt
taccggataaggcgcagcggtcgggctgaacggggggttcgtgcacacagcccagcttggagcg
aacgacctacaccgaactgagatacctacagcgtgagctatgagaaagcgccacgcttcccgaa
gggagaaaggcggacaggtatccggtaagcggcagggtcggaacaggagagcgcacgagggagc
ttccagggggaaacgcctggtatctttatagtcctgtcgggtttcgccacctctgacttgagcg
tcgatttttgtgatgctcgtcaggggggcggagcctatggaaaaacgccagcaacgcggccttt
ttacggttcctggccttttgctggccttttgctcacatgt

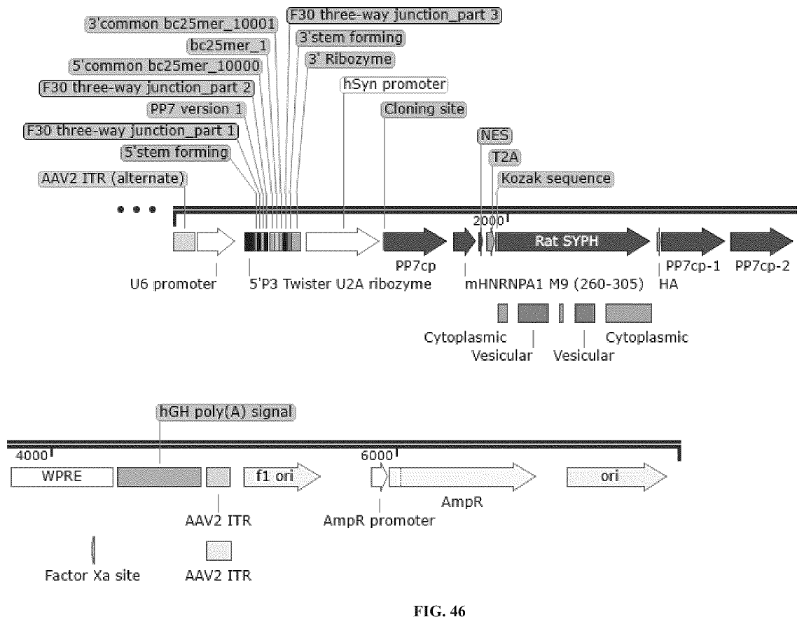

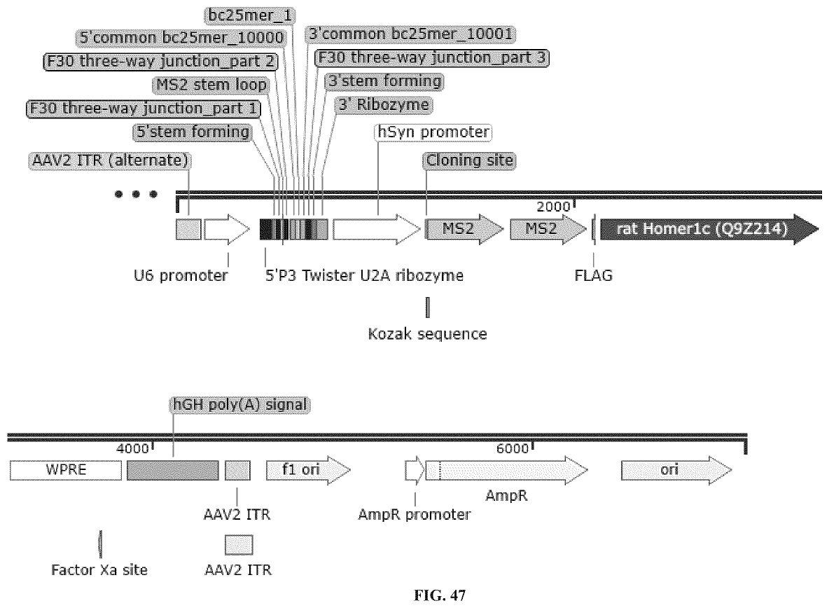

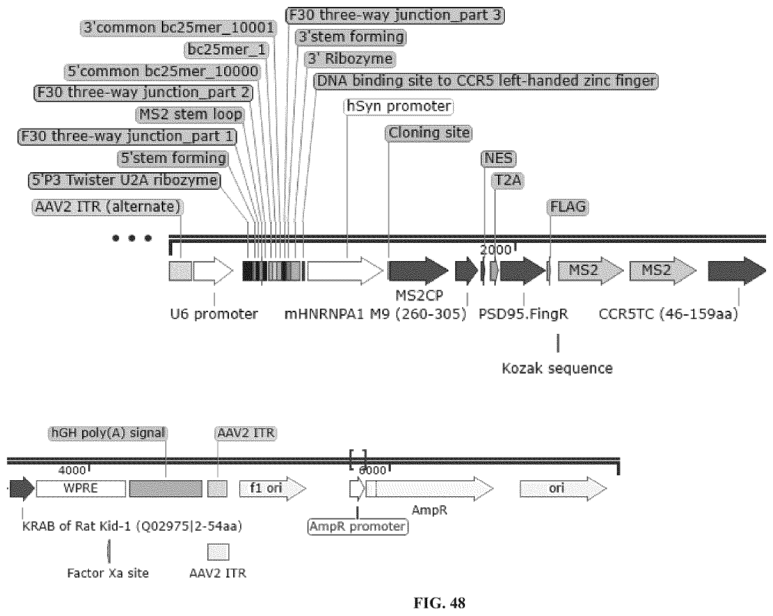

Plasmid encoding GC-M9 (see FIG. 9A) (see FIG. 46 for a map of the plasmid)

(SEQ ID NO: 89)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggccctgcgtatgagtgcaagtgggttttaggaccaggatgaggcgg
ggtgggggtgcctacctgacgaccgaccccgacccactggacaagcacccaacccccattcccc
aaattgcgcatcccctatcagagagggggaggggaaacaggatgcggcgaggcgcgtgcgcact
gccagcttcagcaccgcggacagtgccttcgcccccgcctggcggcgcgcgccaccgccgcctc
agcactgaaggcgcgctgacgtcactcgccggtcccccgcaaactccccttcccggccaccttg
gtcgcgtccgcgccgccgccggcccagccggaccgcaccacgcgaggcgcgagataggggggca
cgggcgcgaccatctgcgctgcggcgccggcgactcagcgctgcctcagtctgcggtgggcagc
ggaggagtcgtgtcgtgcctgagagcgcagtcgagaaggtaccGGATCCatgtccaaaaccatc
gttctttcggtcggcgaggctactcgcactctgactgagatccagtccaccgcagaccgtcaga
tcttcgaagagaaggtcgggcctctggtgggtcggctgcgcctcacggcttcgctccgtcaaaa
cggagccaagaccgcgtatcgcgtcaacctaaaactggatcaggcggacgtcgttgattgctcc
accagcgtctgcggcgagcttccgaaagtgcgctacactcaggtatggtcgcacgacgtgacaa
tcgttgcgaatagcaccgaggcctcgcgcaaatcgttgtacgatttgaccaagtccctcgtcgc
gacctcgcaggtcgaagatcttgtcgtcaaccttgtgccgctgggccgtggcggtggcggatct
ggcggcggtggtagcAATGATTTTGGCAATTACAACAATCAGTCTTCCAATTTTGGGCCGATGA
AGGGAGGAAACTTTGGAGGCAGGAGCTCTGGCCCTTATGGTGGTGGAGGCCAGTACTTTGCTAA
ACCACGGAACCAAGGTGGCTATGGCGGAGGTGGTTCTctgcctccacttgaaagactgacactg
ggctcaggaggatctggtggttctgagggcagaggaagtcttctaacatgcggtgacgtggagg
agaatcccggccctGCCACCatggacgtggtgaatcagctggtggctgggggtcagttccgggt
ggtcaaggagccccttggcttcgtgaaggtgctgcagtgggtctttgccatcttcgcctttgct
acgtgtggcagctacaccggggagcttcggctgagcgtggagtgtgccaacaagacggagagtg
ccctcaacatcgaagttgaattcgagtaccccttcaggctgcaccaagtgtactttgatgcacc
ctcctgcgtcaaagggggcactaccaagatcttcctggttggggactactcctcgtcggctgaa
ttctttgtcaccgtggctgtgtttgccttcctctactccatgggggccctggccacctacatct
tcctgcagaacaagtaccgagagaacaacaaagggcctatgatggactttctggctacagccgt
gttcgctttcatgtggctagttagttcatcagcctgggccaaaggcctgtccgatgtgaagatg
gccacggacccagagaacattatcaaggagatgcccatgtgccgccagacagggaacacatgca
aggaactgagggaccctgtgacttcaggactcaacacctcagtggtgtttggcttcctgaacct
ggtgctctgggttggcaacttatggttcgtgttcaaggagacaggctgggcagccccattcatg
cgcgcacctccaggcgccccggaaaagcaaccagcacctggcgatgcctacggcgatgcgggct
acgggcagggccccggaggctatgggccccaggactcctacgggcctcagggtggttatcaacc
cgattacgggcagccagccagcggtggcggtggctacgggcctcagggcgactatgggcagcaa
ggctatggccaacagggtgcgcccacctccttctccaatcagatgaaaaccggtggtggcggca
gtggtggcggcagcTATCCATATGATGTTCCAGATTATGCTGCCACCATGTCCAAAACCATCGT
TCTTTCGGTCGGCGAGGCTACTCGCACTCTGACTGAGATCCAGTCCACCGCAGACCGTCAGATC
TTCGAAGAGAAGGTCGGGCCTCTGGTGGGTCGGCTGCGCCTCACGGCTTCGCTCCGTCAAAACG
GAGCCAAGACCGCGTATCGCGTCAACCTAAAACTGGATCAGGCGGACGTCGTTGATTGCTCCAC
CAGCGTCTGCGGCGAGCTTCCGAAAGTGCGCTACACTCAGGTATGGTCGCACGACGTGACAATC
GTTGCGAATAGCACCGAGGCCTCGCGCAAATCGTTGTACGATTTGACCAAGTCCCTCGTCGCGA
CCTCGCAGGTCGAAGATCTTGTCGTCAACCTTGTGCCGCTGGGCCGTCGTGCGGACCCGCTAGC
CTCCTgcggccgcTCCAAAACCATCGTTCTTTCGGTCGGCGAGGCTACTCGCACTCTGACTGAG
ATCCAGTCCACCGCAGACCGTCAGATCTTCGAAGAGAAGGTCGGGCCTCTGGTGGGTCGGCTGC
GCCTCACGGCTTCGCTCCGTCAAAACGGAGCCAAGACCGCGTATCGCGTCAACCTAAAACTGGA
TCAGGCGGACGTCGTTGATTGCTCCACCAGCGTCTGCGGCGAGCTTCCGAAAGTGCGCTACACT
CAGGTATGGTCGCACGACGTGACAATCGTTGCGAATAGCACCGAGGCCTCGCGCAAATCGTTGT
ACGATTTGACCAAGTCCCTCGTCGCGACCTCGCAGGTCGAAGATCTTGTCGTCAACCTTGTGCC
GCTGGGCCGTCGTGCGGACCCGCTAGCCTCCACGCGTGATtgaaagcttatcgataatcaacct
ctggattacaaaatttgtgaaagattgactggtattcttaactatgttgctccttttacgctat
gtggatacgctgctttaatgcctttgtatcatgctattgcttcccgtatggctttcattttctc
ctccttgtataaatcctggttgctgtctctttatgaggagttgtggcccgttgtcaggcaacgt
ggcgtggtgtgcactgtgtttgctgacgcaacccccactggttggggcattgccaccacctgtc
agctcctttccgggactttcgctttccccctccctattgccacggcggaactcatcgccgcctg
ccttgcccgctgctggacaggggctcggctgttgggcactgacaattccgtggtgttgtcgggg
aaatcatcgtcctttccttggctgctcgcctatgttgccacctggattctgcgcgggacgtcct
tctgctacgtcccttcggccctcaatccagcggaccttccttcccgcggcctgctgccggctct
gcggcctcttccgcgtcttcgccttcgccctcagacgagtcggatctccctttgggccgcctcc
ccgcatcgataccgagcgctgctcgagagatctacgggtggcatccctgtgacccctccccagt
gcctctcctggccctggaagttgccactccagtgcccaccagccttgtcctaataaaattaagt
tgcatcattttgtctgactaggtgtccttctataatattatggggtggaggggggtggtatgga
gcaaggggcaagttgggaagacaacctgtagggcctgcggggtctattgggaaccaagctggag
tgcagtggcacaatcttggctcactgcaatctccgcctcctgggttcaagcgattctcctgcct
cagcctcccgagttgttgggattccaggcatgcatgaccaggctcagctaatttttgttttttt
ggtagagacggggtttcaccatattggccaggctggtctccaactcctaatctcaggtgatcta
cccaccttggcctcccaaattgctgggattacaggcgtgaaccactgctcccttccctgtcctt
ctgattttgtaggtaaccacgtgcggaccgagcggccgcaggaacccctagtgatggagttggc
cactccctctctgcgcgctcgctcgctcactgaggccgggcgaccaaaggtcgcccgacgcccg
ggctttgcccgggcggcctcagtgagcgagcgagcgcgcagctgcctgcaggggcgcctgatgc
ggtattttctccttacgcatctgtgcggtatttcacaccgcatacgtcaaagcaaccatagtac
gcgccctgtagcggcgcattaagcgcggcgggtgtggtggttacgcgcagcgtgaccgctacac
ttgccagcgccttagcgcccgctcctttcgctttcttcccttcctttctcgccacgttcgccgg
ctttccccgtcaagctctaaatcgggggctccctttagggttccgatttagtgctttacggcac
ctcgaccccaaaaaacttgatttgggtgatggttcacgtagtgggccatcgccctgatagacgg
tttttcgccctttgacgttggagtccacgttctttaatagtggactcttgttccaaactggaac
aacactcaactctatctcgggctattcttttgatttataagggattttgccgatttcggtctat
tggttaaaaaatgagctgatttaacaaaaatttaacgcgaattttaacaaaatattaacgttta
caattttatggtgcactctcagtacaatctgctctgatgccgcatagttaagccagccccgaca
cccgccaacacccgctgacgcgccctgacgggcttgtctgctcccggcatccgcttacagacaa
gctgtgaccgtctccgggagctgcatgtgtcagaggttttcaccgtcatcaccgaaacgcgcga
gacgaaagggcctcgtgatacgcctatttttataggttaatgtcatgataataatggtttctta
gacgtcaggtggcacttttcggggaaatgtgcgcggaacccctatttgtttatttttctaaata
cattcaaatatgtatccgctcatgagacaataaccctgataaatgcttcaataatattgaaaaa
ggaagagtatgagtattcaacatttccgtgtcgcccttattcccttttttgcggcattttgcct
tcctgtttttgctcacccagaaacgctggtgaaagtaaaagatgctgaagatcagttgggtgca
cgagtgggttacatcgaactggatctcaacagcggtaagatccttgagagttttcgccccgaag
aacgttttccaatgatgagcacttttaaagttctgctatgtggcgcggtattatcccgtattga
cgccgggcaagagcaactcggtcgccgcatacactattctcagaatgacttggttgagtactca
ccagtcacagaaaagcatcttacggatggcatgacagtaagagaattatgcagtgctgccataa
ccatgagtgataacactgcggccaacttacttctgacaacgatcggaggaccgaaggagctaac
cgcttttttgcacaacatgggggatcatgtaactcgccttgatcgttgggaaccggagctgaat
gaagccataccaaacgacgagcgtgacaccacgatgcctgtagcaatggcaacaacgttgcgca
aactattaactggcgaactacttactctagcttcccggcaacaattaatagactggatggaggc
ggataaagttgcaggaccacttctgcgctcggcccttccggctggctggtttattgctgataaa
tctggagccggtgagcgtgggtctcgcggtatcattgcagcactggggccagatggtaagccct
cccgtatcgtagttatctacacgacggggagtcaggcaactatggatgaacgaaatagacagat
cgctgagataggtgcctcactgattaagcattggtaactgtcagaccaagtttactcatatata
ctttagattgatttaaaacttcatttttaatttaaaaggatctaggtgaagatcctttttgata
atctcatgaccaaaatcccttaacgtgagttttcgttccactgagcgtcagaccccgtagaaaa
gatcaaaggatcttcttgagatcctttttttctgcgcgtaatctgctgcttgcaaacaaaaaaa
ccaccgctaccagcggtggtttgtttgccggatcaagagctaccaactctttttccgaaggtaa
ctggcttcagcagagcgcagataccaaatactgttcttctagtgtagccgtagttaggccacca
cttcaagaactctgtagcaccgcctacatacctcgctctgctaatcctgttaccagtggctgct
gccagtggcgataagtcgtgtcttaccgggttggactcaagacgatagttaccggataaggcgc
agcggtcgggctgaacggggggttcgtgcacacagcccagcttggagcgaacgacctacaccga
actgagatacctacagcgtgagctatgagaaagcgccacgcttcccgaagggagaaaggcggac
aggtatccggtaagcggcagggtcggaacaggagagcgcacgagggagcttccagggggaaacg
cctggtatctttatagtcctgtcgggtttcgccacctctgacttgagcgtcgatttttgtgatg
ctcgtcaggggggcggagcctatggaaaaacgccagcaacgcggcctttttacggttcctggcc
ttttgctggccttttgctcacatgt

Plasmid encoding GD (see FIG. 9B) (see FIG. 47 for a map of the plasmid)

(SEQ ID NO: 90)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgacatgaggatcacccatgtcggtccgatactctgatgatgggtcccCCTAGG
TTAAGGATGCACCGACGGGACGTTCTATGAGGACGAATCTCCCGCTTATAAGATTCTATAAACT
GTGCGGTCCTTCAATTGgggtcccatcattcatggcaagtggccgcggtcggcgtggactgtag
aacactgccaatgccggtcccaagcccggataaaaGTGGAGGGTACAGTCCACGCtttttttct
agactgcagagggccctgcgtatgagtgcaagtgggttttaggaccaggatgaggcggggtggg
ggtgcctacctgacgaccgaccccgacccactggacaagcacccaacccccattccccaaattg
cgcatcccctatcagagagggggaggggaaacaggatgcggcgaggcgcgtgcgcactgccagc
ttcagcaccgcggacagtgccttcgcccccgcctggcggcgcgcgccaccgccgcctcagcact
gaaggcgcgctgacgtcactcgccggtcccccgcaaactccccttcccggccaccttggtcgcg
tccgcgccgccgccggcccagccggaccgcaccacgcgaggcgcgagataggggggcacgggcg
cgaccatctgcgctgcggcgccggcgactcagcgctgcctcagtctgcggtgggcagcggagga
gtcgtgtcgtgcctgagagcgcagtcgagaaggtaccGGATCCctagccaccatggccagcaac
ttcacccagtttgtgctggtggacaatggcgggacaggcgatgtgactgtggctccctccaact
tcgccaatggggtggctgagtggatcagctccaacagtcggtcacaggcctacaaggtgacctg
cagcgtgcggcagtctagtgctcagaagagaaagtacacaattaaggtggaggtgcccaaagtg
gccacccagacagtgggaggagtggaactgcctgtggctgctcggagatcctacctgaacatgg
agctgactatccctattttcgccaccaattctgactgtgaactgatcgtgaaggctatgcaggg
actgctgaaagatggcaaccccatcccttctgccattgccgctaatagtggaatctatggcgcc
cctgggattcacccagggatgatggcttctaacttcacccaatttgtgctggtcgataatgggg
gaaccggagatgtgacagtggccccaagtaactttgccaatggcgtggctgaatggatctcaag
caatagccggtcccaggcctacaaagtgacttgctccgtgaggcagtcctctgctcagaagcgc
aaatatacaattaaggtcgaagtgccaaaagtggccactcagaccgtcggcggagtggaactgc
ccgtggctgctaggcgcagttacctgaatatggaactgacaatccctatttttgccactaattc
agattgcgagctgattgtgaaggctatgcaggggctgctgaaagatggaaacccaatcccctca
gccattgccgctaatagcggcatctacctcgagccaaccggtgattacaaggatgacgacgata
agggtggtggtggtgggtcgaccatgggggaacaacctatcttcagcactcgagctcatgtctt
ccagatcgacccaaacacaaagaagaactgggtacccaccagcaagcatgcagttactgtgtct
tatttctatgacagcacaaggaatgtgtataggataatcagtttagacggctcaaaggcaataa
taaatagcaccatcactccaaacatgacatttactaaaacatctcaaaagtttggccaatgggc
tgatagccgggcaaacactgtttatggactgggattctcctctgagcatcatctctcaaaattt
gcagaaaagtttcaggaatttaaagaagctgctcggctggcaaaggagaagtcgcaggagaaga
tggaactgaccagtaccccttcacaggaatcagcaggaggagatcttcagtctcctttaacacc
agaaagtatcaatgggacagatgatgagagaacacccgatgtgacacagaactcagagccaagg
gctgagccagctcagaatgcattgccattttcacatagtgccggggatcgaacccagggcctct
ctcatgctagttcagccatcagcaaacactgggaggctgaactagccacgctcaaggggaacaa
tgccaagctcaccgcagcgctgctggagtccactgccaacgtgaagcagtggaagcaacagctg
gctgcctaccaggaggaggcagagcggctgcacaagcgggtcacggagctggaatgtgttagta
gtcaagcaaacgcggtgcacagccacaagacagagctgagtcagacagtgcaggagctggaaga
gaccctaaaagtaaaggaagaggaaatagaaagattaaaacaagaaattgataacgccagagaa
cttcaagaacagagggactctttgactcagaaactacaggaagttgagattcgaaataaagacc
tggaggggcagctgtcggagctggagcagcgcctggagaagagccagagcgagcaggacgcttt
ccgcagtaacctgaagactctcctagagattctggacgggaaaatatttgaactaacagaattg
cgggataatttggccaagctactagaatgcagctaaaagcttatcgataatcaacctctggatt
acaaaatttgtgaaagattgactggtattcttaactatgttgctccttttacgctatgtggata
cgctgctttaatgcctttgtatcatgctattgcttcccgtatggctttcattttctcctccttg
tataaatcctggttgctgtctctttatgaggagttgtggcccgttgtcaggcaacgtggcgtgg
tgtgcactgtgtttgctgacgcaacccccactggttggggcattgccaccacctgtcagctcct
ttccgggactttcgctttccccctccctattgccacggcggaactcatcgccgcctgccttgcc
cgctgctggacaggggctcggctgttgggcactgacaattccgtggtgttgtcggggaaatcat
cgtcctttccttggctgctcgcctatgttgccacctggattctgcgcgggacgtccttctgcta
cgtcccttcggccctcaatccagcggaccttccttcccgcggcctgctgccggctctgcggcct
cttccgcgtcttcgccttcgccctcagacgagtcggatctccctttgggccgcctccccgcatc
gataccgagcgctgctcgagagatctacgggtggcatccctgtgacccctccccagtgcctctc
ctggccctggaagttgccactccagtgcccaccagccttgtcctaataaaattaagttgcatca
ttttgtctgactaggtgtccttctataatattatggggtggaggggggtggtatggagcaaggg
gcaagttgggaagacaacctgtagggcctgcggggtctattgggaaccaagctggagtgcagtg
gcacaatcttggctcactgcaatctccgcctcctgggttcaagcgattctcctgcctcagcctc
ccgagttgttgggattccaggcatgcatgaccaggctcagctaatttttgtttttttggtagag
acggggtttcaccatattggccaggctggtctccaactcctaatctcaggtgatctacccacct
tggcctcccaaattgctgggattacaggcgtgaaccactgctcccttccctgtccttctgattt
tgtaggtaaccacgtgcggaccgagcggccgcaggaacccctagtgatggagttggccactccc
tctctgcgcgctcgctcgctcactgaggccgggcgaccaaaggtcgcccgacgcccgggctttg
cccgggcggcctcagtgagcgagcgagcgcgcagctgcctgcaggggcgcctgatgcggtattt
tctccttacgcatctgtgcggtatttcacaccgcatacgtcaaagcaaccatagtacgcgccct
gtagcggcgcattaagcgcggcgggtgtggtggttacgcgcagcgtgaccgctacacttgccag
cgccttagcgcccgctcctttcgctttcttcccttcctttctcgccacgttcgccggctttccc
cgtcaagctctaaatcgggggctccctttagggttccgatttagtgctttacggcacctcgacc
ccaaaaaacttgatttgggtgatggttcacgtagtgggccatcgccctgatagacggtttttcg
ccctttgacgttggagtccacgttctttaatagtggactcttgttccaaactggaacaacactc
aactctatctcgggctattcttttgatttataagggattttgccgatttcggtctattggttaa
aaaatgagctgatttaacaaaaatttaacgcgaattttaacaaaatattaacgtttacaatttt
atggtgcactctcagtacaatctgctctgatgccgcatagttaagccagccccgacacccgcca
acacccgctgacgcgccctgacgggcttgtctgctcccggcatccgcttacagacaagctgtga
ccgtctccgggagctgcatgtgtcagaggttttcaccgtcatcaccgaaacgcgcgagacgaaa
gggcctcgtgatacgcctatttttataggttaatgtcatgataataatggtttcttagacgtca
ggtggcacttttcggggaaatgtgcgcggaacccctatttgtttatttttctaaatacattcaa
atatgtatccgctcatgagacaataaccctgataaatgcttcaataatattgaaaaaggaagag
tatgagtattcaacatttccgtgtcgcccttattcccttttttgcggcattttgccttcctgtt
tttgctcacccagaaacgctggtgaaagtaaaagatgctgaagatcagttgggtgcacgagtgg
gttacatcgaactggatctcaacagcggtaagatccttgagagttttcgccccgaagaacgttt
tccaatgatgagcacttttaaagttctgctatgtggcgcggtattatcccgtattgacgccggg
caagagcaactcggtcgccgcatacactattctcagaatgacttggttgagtactcaccagtca
cagaaaagcatcttacggatggcatgacagtaagagaattatgcagtgctgccataaccatgag
tgataacactgcggccaacttacttctgacaacgatcggaggaccgaaggagctaaccgctttt
ttgcacaacatgggggatcatgtaactcgccttgatcgttgggaaccggagctgaatgaagcca
taccaaacgacgagcgtgacaccacgatgcctgtagcaatggcaacaacgttgcgcaaactatt
aactggcgaactacttactctagcttcccggcaacaattaatagactggatggaggcggataaa
gttgcaggaccacttctgcgctcggcccttccggctggctggtttattgctgataaatctggag
ccggtgagcgtgggtctcgcggtatcattgcagcactggggccagatggtaagccctcccgtat
cgtagttatctacacgacggggagtcaggcaactatggatgaacgaaatagacagatcgctgag
ataggtgcctcactgattaagcattggtaactgtcagaccaagtttactcatatatactttaga
ttgatttaaaacttcatttttaatttaaaaggatctaggtgaagatcctttttgataatctcat
gaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgc
taccagcggtggtttgtttgccggatcaagagctaccaactctttttccgaaggtaactggctt
cagcagagcgcagataccaaatactgttcttctagtgtagccgtagttaggccaccacttcaag
aactctgtagcaccgcctacatacctcgctctgctaatcctgttaccagtggctgctgccagtg
gcgataagtcgtgtcttaccgggttggactcaagacgatagttaccggataaggcgcagcggtc
gggctgaacggggggttcgtgcacacagcccagcttggagcgaacgacctacaccgaactgaga
tacctacagcgtgagctatgagaaagcgccacgcttcccgaagggagaaaggcggacaggtatc
cggtaagcggcagggtcggaacaggagagcgcacgagggagcttccagggggaaacgcctggta
tctttatagtcctgtcgggtttcgccacctctgacttgagcgtcgatttttgtgatgctcgtca
ggggggcggagcctatggaaaaacgccagcaacgcggcctttttacggttcctggccttttgct
ggccttttgctcacatgt

Plasmid encoding GE1-M9 (see FIG. 9B) (see FIG. 48 for a map of the plasmid)

(SEQ ID NO: 91)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgacatgaggatcacccatgtcggtccgatactctgatgatgggtcccCCTAGG
TTAAGGATGCACCGACGGGACGTTCTATGAGGACGAATCTCCCGCTTATAAGATTCTATAAACT
GTGCGGTCCTTCAATTGgggtcccatcattcatggcaagtggccgcggtcggcgtggactgtag
aacactgccaatgccggtcccaagcccggataaaaGTGGAGGGTACAGTCCACGCtttttttct
agagtcatcctcatcctgcagagggccctgcgtatgagtgcaagtgggttttaggaccaggatg
aggcggggtgggggtgcctacctgacgaccgaccccgacccactggacaagcacccaaccccca
ttccccaaattgcgcatcccctatcagagagggggaggggaaacaggatgcggcgaggcgcgtg
cgcactgccagcttcagcaccgcggacagtgccttcgcccccgcctggcggcgcgcgccaccgc
cgcctcagcactgaaggcgcgctgacgtcactcgccggtcccccgcaaactccccttcccggcc
accttggtcgcgtccgcgccgccgccggcccagccggaccgcaccacgcgaggcgcgagatagg
ggggcacgggcgcgaccatctgcgctgcggcgccggcgactcagcgctgcctcagtctgcggtg
ggcagcggaggagtcgtgtcgtgcctgagagcgcagtcgagaaggtaccGGATCCgccaccatg
gcttctaactttactcagttcgttctcgtcgacaatggcggaactggcgacgtgactgtcgccc
caagcaacttcgctaacggggtcgctgaatggatcagctctaactcgcgttcacaggcttacaa
agtaacctgtagcgttcgtcagagctctgcgcagaatcgcaaatacaccatcaaagtcgaggtg
cctaaaggcgcatggcgttcgtacttaaatatggaactaaccattccaattttctccacgaact
ccgactgcgagcttattgttaaggcaatgcaaggtctcctaaaagatggaaacccgattccctc
agcaatcgcagcaaactccggcatctacggcggtggcggatctggcggcggtggtagcAATGAT
TTTGGCAATTACAACAATCAGTCTTCCAATTTTGGGCCGATGAAGGGAGGAAACTTTGGAGGCA
GGAGCTCTGGCCCTTATGGTGGTGGAGGCCAGTACTTTGCTAAACCACGGAACCAAGGTGGCTA
TGGCGGAGGTGGTTCTctgcctccacttgaaagactgacactgggctcaggaggatctggtggt
tctgagggcagaggaagtcttctaacatgcggtgacgtggaggagaatcccggccctgccACCa
tgctcgaagtcaaggaagcatcaccaaccagcatccagatcagctgggtgctccacttgcgcca
cgttcgctactaccgcatcacctacggtgaaactggtggcaatagccctgtccaggaattcacc
gtgcctggcagcaagtccactgctaccatcagcggcctgaaacctggtgtcgactataccatca
cggtgtacgccgtcacgatcttcagcgcctaccgctccgcctggccgccgatctccatcaacta
ccgcaccggaaccgattacaaggatgacgacgataagggtagcggctccagtagatctgggcta
cttaaggccaccatggccagcaacttcacccagtttgtgctggtggacaatggcgggacaggcg
atgtgactgtggctccctccaacttcgccaatggggtggctgagtggatcagctccaacagtcg
gtcacaggcctacaaggtgacctgcagcgtgcggcagtctagtgctcagaagagaaagtacaca
attaaggtggaggtgcccaaagtggccacccagacagtgggaggagtggaactgcctgtggctg
ctcggagatcctacctgaacatggagctgactatccctattttcgccaccaattctgactgtga
actgatcgtgaaggctatgcagggactgctgaaagatggcaaccccatcccttctgccattgcc
gctaatagtggaatctatggcgcccctgggattcacccagggatgatggcttctaacttcaccc
aatttgtgctggtcgataatgggggaaccggagatgtgacagtggccccaagtaactttgccaa
tggcgtggctgaatggatctcaagcaatagccggtcccaggcctacaaagtgacttgctccgtg
aggcagtcctctgctcagaagcgcaaatatacaattaaggtcgaagtgccaaaagtggccactc
agaccgtcggcggagtggaactgcccgtggctgctaggcgcagttacctgaatatggaactgac
aatccctatttttgccactaattcagattgcgagctgattgtgaaggctatgcaggggctgctg
aaagatggaaacccaatcccctcagccattgccgctaatagcggcatctacctcgagccagagc
tctctaggggagctggcgctggagcgggtgcaggggctggctctagattccagtgccggatctg
catgcggaacttcagcgaccggtccaacctgagcaggcacatcagaacccacaccggagaaaag
cccttcgcctgcgacatttgcggccggaagttcgccatcagcagcaacctgaacagccacacca
agatccacactggcagccagaaacctttccagtgcagaatttgtatgagaaactttagcagaag
cgacaacctggccagacacatccggacacatactggtgaaaaaccttttgcctgtgatatctgt
ggcagaaagtttgccacctccggcaatctgacccggcacacaaagattcacctgcggggcagcc
agctatcgattgtcgacgctcctgaacaacgtgaaggtgcttctcaagtttctgtttctgttac
ttttgaagatgttgctgttctttttactcgtgatgaatggaaaaaacttgatctttctcaacgt
tctctttatcgtgaagttatgcttgaaaattattctaatcttgcttctatggcttaaaagctta
tcgataatcaacctctggattacaaaatttgtgaaagattgactggtattcttaactatgttgc
tccttttacgctatgtggatacgctgctttaatgcctttgtatcatgctattgcttcccgtatg
gctttcattttctcctccttgtataaatcctggttgctgtctctttatgaggagttgtggcccg
ttgtcaggcaacgtggcgtggtgtgcactgtgtttgctgacgcaacccccactggttggggcat
tgccaccacctgtcagctcctttccgggactttcgctttccccctccctattgccacggcggaa
ctcatcgccgcctgccttgcccgctgctggacaggggctcggctgttgggcactgacaattccg
tggtgttgtcggggaaatcatcgtcctttccttggctgctcgcctatgttgccacctggattct
gcgcgggacgtccttctgctacgtcccttcggccctcaatccagcggaccttccttcccgcggc
ctgctgccggctctgcggcctcttccgcgtcttcgccttcgccctcagacgagtcggatctccc
tttgggccgcctccccgcatcgataccgagcgctgctcgagagatctacgggtggcatccctgt
gacccctccccagtgcctctcctggccctggaagttgccactccagtgcccaccagccttgtcc
taataaaattaagttgcatcattttgtctgactaggtgtccttctataatattatggggtggag
gggggtggtatggagcaaggggcaagttgggaagacaacctgtagggcctgcggggtctattgg
gaaccaagctggagtgcagtggcacaatcttggctcactgcaatctccgcctcctgggttcaag
cgattctcctgcctcagcctcccgagttgttgggattccaggcatgcatgaccaggctcagcta
atttttgtttttttggtagagacggggtttcaccatattggccaggctggtctccaactcctaa
tctcaggtgatctacccaccttggcctcccaaattgctgggattacaggcgtgaaccactgctc
ccttccctgtccttctgattttgtaggtaaccacgtgcggaccgagcggccgcaggaaccccta
gtgatggagttggccactccctctctgcgcgctcgctcgctcactgaggccgggcgaccaaagg
tcgcccgacgcccgggctttgcccgggcggcctcagtgagcgagcgagcgcgcagctgcctgca
ggggcgcctgatgcggtattttctccttacgcatctgtgcggtatttcacaccgcatacgtcaa
agcaaccatagtacgcgccctgtagcggcgcattaagcgcggcgggtgtggtggttacgcgcag
cgtgaccgctacacttgccagcgccttagcgcccgctcctttcgctttcttcccttcctttctc
gccacgttcgccggctttccccgtcaagctctaaatcgggggctccctttagggttccgattta
gtgctttacggcacctcgaccccaaaaaacttgatttgggtgatggttcacgtagtgggccatc
gccctgatagacggtttttcgccctttgacgttggagtccacgttctttaatagtggactcttg
ttccaaactggaacaacactcaactctatctcgggctattcttttgatttataagggattttgc
cgatttcggtctattggttaaaaaatgagctgatttaacaaaaatttaacgcgaattttaacaa
aatattaacgtttacaattttatggtgcactctcagtacaatctgctctgatgccgcatagtta
agccagccccgacacccgccaacacccgctgacgcgccctgacgggcttgtctgctcccggcat
ccgcttacagacaagctgtgaccgtctccgggagctgcatgtgtcagaggttttcaccgtcatc
accgaaacgcgcgagacgaaagggcctcgtgatacgcctatttttataggttaatgtcatgata
ataatggtttcttagacgtcaggtggcacttttcggggaaatgtgcgcggaacccctatttgtt
tatttttctaaatacattcaaatatgtatccgctcatgagacaataaccctgataaatgcttca
ataatattgaaaaaggaagagtatgagtattcaacatttccgtgtcgcccttattccctttttt
gcggcattttgccttcctgtttttgctcacccagaaacgctggtgaaagtaaaagatgctgaag
atcagttgggtgcacgagtgggttacatcgaactggatctcaacagcggtaagatccttgagag
ttttcgccccgaagaacgttttccaatgatgagcacttttaaagttctgctatgtggcgcggta
ttatcccgtattgacgccgggcaagagcaactcggtcgccgcatacactattctcagaatgact
tggttgagtactcaccagtcacagaaaagcatcttacggatggcatgacagtaagagaattatg
cagtgctgccataaccatgagtgataacactgcggccaacttacttctgacaacgatcggagga
ccgaaggagctaaccgcttttttgcacaacatgggggatcatgtaactcgccttgatcgttggg
aaccggagctgaatgaagccataccaaacgacgagcgtgacaccacgatgcctgtagcaatggc
aacaacgttgcgcaaactattaactggcgaactacttactctagcttcccggcaacaattaata
gactggatggaggcggataaagttgcaggaccacttctgcgctcggcccttccggctggctggt
ttattgctgataaatctggagccggtgagcgtgggtctcgcggtatcattgcagcactggggcc
agatggtaagccctcccgtatcgtagttatctacacgacggggagtcaggcaactatggatgaa
cgaaatagacagatcgctgagataggtgcctcactgattaagcattggtaactgtcagaccaag
tttactcatatatactttagattgatttaaaacttcatttttaatttaaaaggatctaggtgaa
gatcctttttgataatctcatgaccaaaatcccttaacgtgagttttcgttccactgagcgtca
gaccccgtagaaaagatcaaaggatcttcttgagatcctttttttctgcgcgtaatctgctgct
tgcaaacaaaaaaaccaccgctaccagcggtggtttgtttgccggatcaagagctaccaactct
ttttccgaaggtaactggcttcagcagagcgcagataccaaatactgttcttctagtgtagccg
tagttaggccaccacttcaagaactctgtagcaccgcctacatacctcgctctgctaatcctgt
taccagtggctgctgccagtggcgataagtcgtgtcttaccgggttggactcaagacgatagtt
accggataaggcgcagcggtcgggctgaacggggggttcgtgcacacagcccagcttggagcga
acgacctacaccgaactgagatacctacagcgtgagctatgagaaagcgccacgcttcccgaag
ggagaaaggcggacaggtatccggtaagcggcagggtcggaacaggagagcgcacgagggagct
tccagggggaaacgcctggtatctttatagtcctgtcgggtttcgccacctctgacttgagcgt
cgatttttgtgatgctcgtcaggggggcggagcctatggaaaaacgccagcaacgcggcctttt
tacggttcctggccttttgctggccttttgctcacatgt

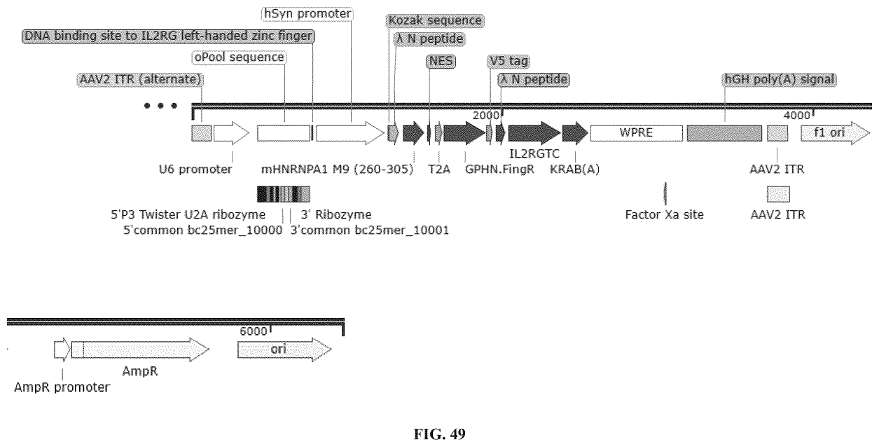

Plasmid encoding GF1-M9 (see FIG. 9B) (see FIG. 49 for a map of the plasmid)

(SEQ ID NO: 92)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccggggccctgaagaagggccccggtccgatactctgatgatgggtcccCCTAGG
TTAAGGATGCACCGACGGGACGTTCTATGAGGACGAATCTCCCGCTTATAAGATTCTATAAACT
GTGCGGTCCTTCAATTGgggtcccatcattcatggcaagtggccgcggtcggcgtggactgtag
aacactgccaatgccggtcccaagcccggataaaaGTGGAGGGTACAGTCCACGCtttttttct
agacttccacagagtctgcagagggccctgcgtatgagtgcaagtgggttttaggaccaggatg
aggcggggtgggggtgcctacctgacgaccgaccccgacccactggacaagcacccaaccccca
ttccccaaattgcgcatcccctatcagagagggggaggggaaacaggatgcggcgaggcgcgtg
cgcactgccagcttcagcaccgcggacagtgccttcgcccccgcctggcggcgcgcgccaccgc
cgcctcagcactgaaggcgcgctgacgtcactcgccggtcccccgcaaactccccttcccggcc
accttggtcgcgtccgcgccgccgccggcccagccggaccgcaccacgcgaggcgcgagatagg
ggggcacgggcgcgaccatctgcgctgcggcgccggcgactcagcgctgcctcagtctgcggtg
ggcagcggaggagtcgtgtcgtgcctgagagcgcagtcgagaaggtaccGCCACCatggacgca
caaacacgacgacgtgagcgtcgcgctgagaaacaagctcaatggaaagctgcaaacggcggtg
gcggatctggcggcggtggtagcAATGATTTTGGCAATTACAACAATCAGTCTTCCAATTTTGG
GCCGATGAAGGGAGGAAACTTTGGAGGCAGGAGCTCTGGCCCTTATGGTGGTGGAGGCCAGTAC
TTTGCTAAACCACGGAACCAAGGTGGCTATGGCGGAGGTGGTTCTctgcctccacttgaaagac
tgacactgggctcaggaggatctggtggttctgagggcagaggaagtcttctaacatgcggtga
cgtggaggagaatcccggccctGGATCCatgctcgaagtcaaggaagcatcaccaaccagcatc
cagatcagctggggcaagtacaaggtcatggttcgctactaccgcatcacctacggtgaaactg
gtggcaatagccctgtccaggaattcaccgtgcctggcagcaagtccactgctaccatcagcag
cctgaaacctggtgtcgactataccatcacggtgtacgccgtcacgatcgaccactggaactac
caggacccgatcccgatctccatcaactaccgcaccggatccggcaagcccatccccaaccccc
tgctgggcctggacagcaccGGCGGAGGTGGTTCTaccggtgacgcacaaacacgacgacgtga
gcgtcgcgctgagaaacaagctcaatggaaagctgcaaacgagctctctagatttcagtgccag
atttgcatgcgcaactttagccgcaaaagcaccctgaccgatcatattcgcacccataccggcg
aaaaaccgtttgcgtgcgatatttgcggccgcaaatttgcggcgcgcagcacccgcaccaccca
taccaaaattcataccggcagccagaaaccgtttcagtgccgcatttgcatgcgcaactttagc
cgcagcgatagcctgagcaaacatattcgcacccataccggcgaaaaaccgtttgcgtgcgata
tttgcggccgcaaatttgcgcagcgcagcaacctgaaagtgcataccaaaattcatctgcgcgg
cagccagctgatcgatggtgtcgacgctcctgaacaacgtgaaggtgcttctcaagtttctgtt
tctgttacttttgaagatgttgctgttctttttactcgtgatgaatggaaaaaacttgatcttt
ctcaacgttctctttatcgtgaagttatgcttgaaaattattctaatcttgcttctatggctta
aaagcttatcgataatcaacctctggattacaaaatttgtgaaagattgactggtattcttaac
tatgttgctccttttacgctatgtggatacgctgctttaatgcctttgtatcatgctattgctt
cccgtatggctttcattttctcctccttgtataaatcctggttgctgtctctttatgaggagtt
gtggcccgttgtcaggcaacgtggcgtggtgtgcactgtgtttgctgacgcaacccccactggt
tggggcattgccaccacctgtcagctcctttccgggactttcgctttccccctccctattgcca
cggcggaactcatcgccgcctgccttgcccgctgctggacaggggctcggctgttgggcactga
caattccgtggtgttgtcggggaaatcatcgtcctttccttggctgctcgcctatgttgccacc
tggattctgcgcgggacgtccttctgctacgtcccttcggccctcaatccagcggaccttcctt
cccgcggcctgctgccggctctgcggcctcttccgcgtcttcgccttcgccctcagacgagtcg
gatctccctttgggccgcctccccgcatcgataccgagcgctgctcgagagatctacgggtggc
atccctgtgacccctccccagtgcctctcctggccctggaagttgccactccagtgcccaccag
ccttgtcctaataaaattaagttgcatcattttgtctgactaggtgtccttctataatattatg
gggtggaggggggtggtatggagcaaggggcaagttgggaagacaacctgtagggcctgcgggg
tctattgggaaccaagctggagtgcagtggcacaatcttggctcactgcaatctccgcctcctg
ggttcaagcgattctcctgcctcagcctcccgagttgttgggattccaggcatgcatgaccagg
ctcagctaatttttgtttttttggtagagacggggtttcaccatattggccaggctggtctcca
actcctaatctcaggtgatctacccaccttggcctcccaaattgctgggattacaggcgtgaac
cactgctcccttccctgtccttctgattttgtaggtaaccacgtgcggaccgagcggccgcagg
aacccctagtgatggagttggccactccctctctgcgcgctcgctcgctcactgaggccgggcg
accaaaggtcgcccgacgcccgggctttgcccgggcggcctcagtgagcgagcgagcgcgcagc
tgcctgcaggggcgcctgatgcggtattttctccttacgcatctgtgcggtatttcacaccgca
tacgtcaaagcaaccatagtacgcgccctgtagcggcgcattaagcgcggcgggtgtggtggtt
acgcgcagcgtgaccgctacacttgccagcgccttagcgcccgctcctttcgctttcttccctt
cctttctcgccacgttcgccggctttccccgtcaagctctaaatcgggggctccctttagggtt
ccgatttagtgctttacggcacctcgaccccaaaaaacttgatttgggtgatggttcacgtagt
gggccatcgccctgatagacggtttttcgccctttgacgttggagtccacgttctttaatagtg
gactcttgttccaaactggaacaacactcaactctatctcgggctattcttttgatttataagg
gattttgccgatttcggtctattggttaaaaaatgagctgatttaacaaaaatttaacgcgaat
tttaacaaaatattaacgtttacaattttatggtgcactctcagtacaatctgctctgatgccg
catagttaagccagccccgacacccgccaacacccgctgacgcgccctgacgggcttgtctgct
cccggcatccgcttacagacaagctgtgaccgtctccgggagctgcatgtgtcagaggttttca
ccgtcatcaccgaaacgcgcgagacgaaagggcctcgtgatacgcctatttttataggttaatg
tcatgataataatggtttcttagacgtcaggtggcacttttcggggaaatgtgcgcggaacccc
tatttgtttatttttctaaatacattcaaatatgtatccgctcatgagacaataaccctgataa
atgcttcaataatattgaaaaaggaagagtatgagtattcaacatttccgtgtcgcccttattc
ccttttttgcggcattttgccttcctgtttttgctcacccagaaacgctggtgaaagtaaaaga
tgctgaagatcagttgggtgcacgagtgggttacatcgaactggatctcaacagcggtaagatc
cttgagagttttcgccccgaagaacgttttccaatgatgagcacttttaaagttctgctatgtg
gcgcggtattatcccgtattgacgccgggcaagagcaactcggtcgccgcatacactattctca
gaatgacttggttgagtactcaccagtcacagaaaagcatcttacggatggcatgacagtaaga
gaattatgcagtgctgccataaccatgagtgataacactgcggccaacttacttctgacaacga
tcggaggaccgaaggagctaaccgcttttttgcacaacatgggggatcatgtaactcgccttga
tcgttgggaaccggagctgaatgaagccataccaaacgacgagcgtgacaccacgatgcctgta
gcaatggcaacaacgttgcgcaaactattaactggcgaactacttactctagcttcccggcaac
aattaatagactggatggaggcggataaagttgcaggaccacttctgcgctcggcccttccggc
tggctggtttattgctgataaatctggagccggtgagcgtgggtctcgcggtatcattgcagca
ctggggccagatggtaagccctcccgtatcgtagttatctacacgacggggagtcaggcaacta
tggatgaacgaaatagacagatcgctgagataggtgcctcactgattaagcattggtaactgtc
agaccaagtttactcatatatactttagattgatttaaaacttcatttttaatttaaaaggatc
taggtgaagatcctttttgataatctcatgaccaaaatcccttaacgtgagttttcgttccact
gagcgtcagaccccgtagaaaagatcaaaggatcttcttgagatcctttttttctgcgcgtaat
ctgctgcttgcaaacaaaaaaaccaccgctaccagcggtggtttgtttgccggatcaagagcta
ccaactctttttccgaaggtaactggcttcagcagagcgcagataccaaatactgttcttctag
tgtagccgtagttaggccaccacttcaagaactctgtagcaccgcctacatacctcgctctgct
aatcctgttaccagtggctgctgccagtggcgataagtcgtgtcttaccgggttggactcaaga
cgatagttaccggataaggcgcagcggtcgggctgaacggggggttcgtgcacacagcccagct
tggagcgaacgacctacaccgaactgagatacctacagcgtgagctatgagaaagcgccacgct
tcccgaagggagaaaggcggacaggtatccggtaagcggcagggtcggaacaggagagcgcacg
agggagcttccagggggaaacgcctggtatctttatagtcctgtcgggtttcgccacctctgac
ttgagcgtcgatttttgtgatgctcgtcaggggggcggagcctatggaaaaacgccagcaacgc
ggcctttttacggttcctggccttttgctggccttttgctcacatgt

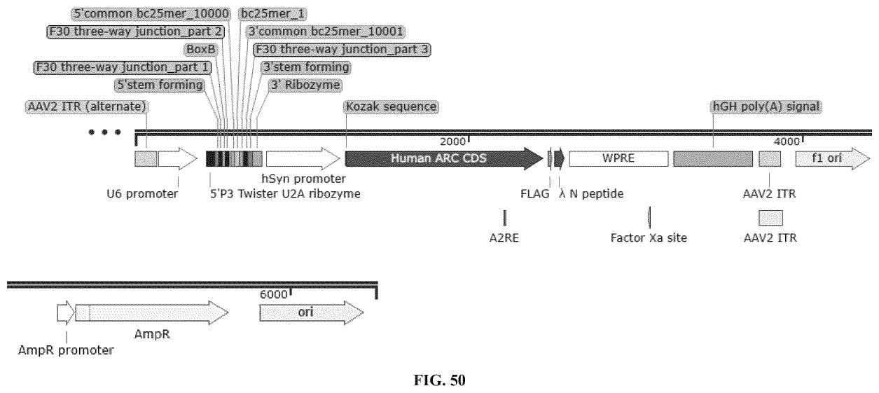

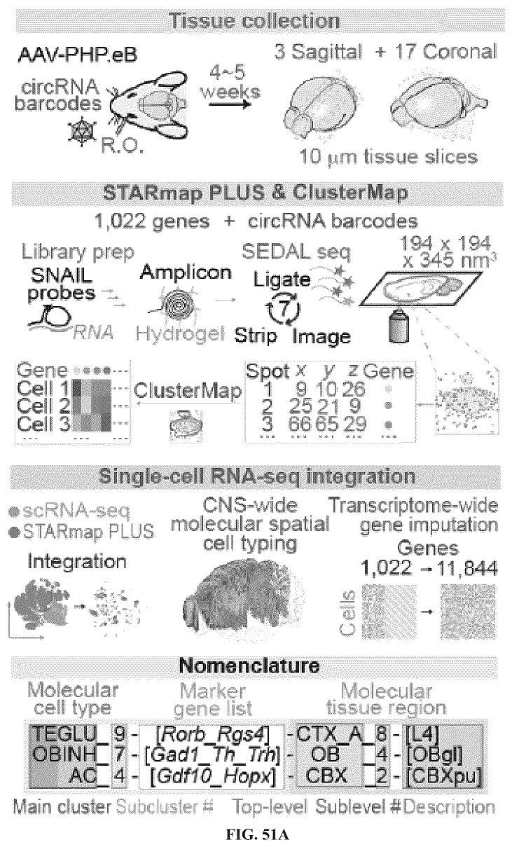

Plasmid encoding GK (see FIG. 9D) (see FIG. 50 for a map of the plasmid)

(SEQ ID NO: 93)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccggggccctgaagaagggccccggtccgatactctgatgatgggtcccCCTAGG
TTAAGGATGCACCGACGGGACGTTCTATGAGGACGAATCTCCCGCTTATAAGATTCTATAAACT
GTGCGGTCCTTCAATTGgggtcccatcattcatggcaagtggccgcggtcggcgtggactgtag
aacactgccaatgccggtcccaagcccggataaaaGTGGAGGGTACAGTCCACGCtttttttct
agactgcagagggccctgcgtatgagtgcaagtgggttttaggaccaggatgaggcggggtggg
ggtgcctacctgacgaccgaccccgacccactggacaagcacccaacccccattccccaaattg
cgcatcccctatcagagagggggaggggaaacaggatgcggcgaggcgcgtgcgcactgccagc
ttcagcaccgcggacagtgccttcgcccccgcctggcggcgcgcgccaccgccgcctcagcact
gaaggcgcgctgacgtcactcgccggtcccccgcaaactccccttcccggccaccttggtcgcg
tccgcgccgccgccggcccagccggaccgcaccacgcgaggcgcgagataggggggcacgggcg
cgaccatctgcgctgcggcgccggcgactcagcgctgcctcagtctgcggtgggcagcggagga
gtcgtgtcgtgcctgagagcgcagtcgagaaggtaccggatccGCCACCatgGAGCTGGACCAC
CGGACCAGCGGCGGGCTCCACGCCTACCCCGGGCCGCGGGGGGGGCAGGTGGCCAAGCCCAACG
TGATCCTGCAGATCGGGAAGTGCCGGGCCGAGATGCTGGAGCACGTGCGGCGGACGCACCGGCA
CCTGCTGGCCGAGGTGTCCAAGCAGGTGGAGCGCGAGCTGAAGGGGCTGCACCGGTCGGTCGGG
AAGCTGGAGAGCAACCTGGACGGCTACGTGCCCACGAGCGACTCGCAGCGCTGGAAGAAGTCCA
TCAAGGCCTGCCTGTGCCGCTGCCAGGAGACCATCGCCAACCTGGAGCGCTGGGTCAAGCGCGA
GATGCACGTGTGGCGCGAGGTGTTCTACCGCCTGGAGCGCTGGGCCGACCGCCTGGAGTCCACG
GGCGGCAAGTACCCGGTGGGCAGCGAGTCAGCCCGCCACACCGTTTCCGTGGGCGTGGGGGGTC
CCGAGAGCTACTGCCACGAGGCAGACGGCTACGACTACACCGTCAGCCCCTACGCCATCACCCC
GCCCCCAGCCGCTGGCGAGCTGCCCGGGCAGGAGCCCGCCGAGGCCCAGCAGTACCAGCCGTGG
GTCCCCGGCGAGGACGGGCAGCCCAGCCCCGGCGTGGACACGCAGATCTTCGAGGACCCTCGAG
AGTTCCTGAGCCACCTAGAGGAGTACTTGCGGCAGGTGGGCGGCTCTGAGGAGTACTGGCTGTC
CCAGATCCAGAATCACATGAACGGGCCGGCCAAGAAGTGGTGGGAGTTCAAGCAGGGCTCCGTG
AAGAACTGGGTGGAGTTCAAGAAGGAGTTCCTGCAGTACAGCGAGGGCACGCTGTCCCGAGAGG
CCATCCAGCGCGAGCTGGACCTGCCGCAGAAGCAGGGCGAGCCGCTGGACCAGTTCCTGTGGCG
CAAGCGGGACCTGTACCAGACGCTCTACGTGGACGCGGACGAGGAGGAGATCATCCAGTACGTG
GTGGGCACCCTGCAGCCCAAGCTCAAGCGTTTCCTGCGCCACCCCCTGCCCAAGACCCTGGAGC
AGCTCATCCAGAGGGGCATGGAGGTGCAGGATGACCTGGAGCAGGCGGCCGAGCCGGCCGGCCC
CCACCTCCCGGTGGAGGATGAGGCGGAGACCCTCACGCCCGCCCCCAACAGCGAGTCCGTGGCC
AGTGACCGGACCCAGCCCGAGggctcaggaggatctggtggttctgattacaaggatgacgacg
ataagggcggaggtggttctgacgcacaaacacgacgacgtgagcgtcgcgctgagaaacaagc
tcaatggaaagctgcaaacTAGgaattcgatatcaagcttatcgataatcaacctctggattac
aaaatttgtgaaagattgactggtattcttaactatgttgctccttttacgctatgtggatacg
ctgctttaatgcctttgtatcatgctattgcttcccgtatggctttcattttctcctccttgta
taaatcctggttgctgtctctttatgaggagttgtggcccgttgtcaggcaacgtggcgtggtg
tgcactgtgtttgctgacgcaacccccactggttggggcattgccaccacctgtcagctccttt
ccgggactttcgctttccccctccctattgccacggcggaactcatcgccgcctgccttgcccg
ctgctggacaggggctcggctgttgggcactgacaattccgtggtgttgtcggggaaatcatcg
tcctttccttggctgctcgcctatgttgccacctggattctgcgcgggacgtccttctgctacg
tcccttcggccctcaatccagcggaccttccttcccgcggcctgctgccggctctgcggcctct
tccgcgtcttcgccttcgccctcagacgagtcggatctccctttgggccgcctccccgcatcga
taccgagcgctgctcgagagatctacgggtggcatccctgtgacccctccccagtgcctctcct
ggccctggaagttgccactccagtgcccaccagccttgtcctaataaaattaagttgcatcatt
ttgtctgactaggtgtccttctataatattatggggtggaggggggtggtatggagcaaggggc
aagttgggaagacaacctgtagggcctgcggggtctattgggaaccaagctggagtgcagtggc
acaatcttggctcactgcaatctccgcctcctgggttcaagcgattctcctgcctcagcctccc
gagttgttgggattccaggcatgcatgaccaggctcagctaatttttgtttttttggtagagac
ggggtttcaccatattggccaggctggtctccaactcctaatctcaggtgatctacccaccttg
gcctcccaaattgctgggattacaggcgtgaaccactgctcccttccctgtccttctgattttg
taggtaaccacgtgcggaccgagcggccgcaggaacccctagtgatggagttggccactccctc
tctgcgcgctcgctcgctcactgaggccgggcgaccaaaggtcgcccgacgcccgggctttgcc
cgggcggcctcagtgagcgagcgagcgcgcagctgcctgcaggggcgcctgatgcggtattttc
tccttacgcatctgtgcggtatttcacaccgcatacgtcaaagcaaccatagtacgcgccctgt
agcggcgcattaagcgcggcgggtgtggtggttacgcgcagcgtgaccgctacacttgccagcg
ccttagcgcccgctcctttcgctttcttcccttcctttctcgccacgttcgccggctttccccg
tcaagctctaaatcgggggctccctttagggttccgatttagtgctttacggcacctcgacccc
aaaaaacttgatttgggtgatggttcacgtagtgggccatcgccctgatagacggtttttcgcc
ctttgacgttggagtccacgttctttaatagtggactcttgttccaaactggaacaacactcaa
ctctatctcgggctattcttttgatttataagggattttgccgatttcggtctattggttaaaa
aatgagctgatttaacaaaaatttaacgcgaattttaacaaaatattaacgtttacaattttat
ggtgcactctcagtacaatctgctctgatgccgcatagttaagccagccccgacacccgccaac
acccgctgacgcgccctgacgggcttgtctgctcccggcatccgcttacagacaagctgtgacc
gtctccgggagctgcatgtgtcagaggttttcaccgtcatcaccgaaacgcgcgagacgaaagg
gcctcgtgatacgcctatttttataggttaatgtcatgataataatggtttcttagacgtcagg
tggcacttttcggggaaatgtgcgcggaacccctatttgtttatttttctaaatacattcaaat
atgtatccgctcatgagacaataaccctgataaatgcttcaataatattgaaaaaggaagagta
tgagtattcaacatttccgtgtcgcccttattcccttttttgcggcattttgccttcctgtttt
tgctcacccagaaacgctggtgaaagtaaaagatgctgaagatcagttgggtgcacgagtgggt
tacatcgaactggatctcaacagcggtaagatccttgagagttttcgccccgaagaacgttttc
caatgatgagcacttttaaagttctgctatgtggcgcggtattatcccgtattgacgccgggca
agagcaactcggtcgccgcatacactattctcagaatgacttggttgagtactcaccagtcaca
gaaaagcatcttacggatggcatgacagtaagagaattatgcagtgctgccataaccatgagtg
ataacactgcggccaacttacttctgacaacgatcggaggaccgaaggagctaaccgctttttt
gcacaacatgggggatcatgtaactcgccttgatcgttgggaaccggagctgaatgaagccata
ccaaacgacgagcgtgacaccacgatgcctgtagcaatggcaacaacgttgcgcaaactattaa
ctggcgaactacttactctagcttcccggcaacaattaatagactggatggaggcggataaagt
tgcaggaccacttctgcgctcggcccttccggctggctggtttattgctgataaatctggagcc
ggtgagcgtgggtctcgcggtatcattgcagcactggggccagatggtaagccctcccgtatcg
tagttatctacacgacggggagtcaggcaactatggatgaacgaaatagacagatcgctgagat
aggtgcctcactgattaagcattggtaactgtcagaccaagtttactcatatatactttagatt
gatttaaaacttcatttttaatttaaaaggatctaggtgaagatcctttttgataatctcatga
ccaaaatcccttaacgtgagttttcgttccactgagcgtcagaccccgtagaaaagatcaaagg
atcttcttgagatcctttttttctgcgcgtaatctgctgcttgcaaacaaaaaaaccaccgcta
ccagcggtggtttgtttgccggatcaagagctaccaactctttttccgaaggtaactggcttca
gcagagcgcagataccaaatactgttcttctagtgtagccgtagttaggccaccacttcaagaa
ctctgtagcaccgcctacatacctcgctctgctaatcctgttaccagtggctgctgccagtggc
gataagtcgtgtcttaccgggttggactcaagacgatagttaccggataaggcgcagcggtcgg
gctgaacggggggttcgtgcacacagcccagcttggagcgaacgacctacaccgaactgagata
cctacagcgtgagctatgagaaagcgccacgcttcccgaagggagaaaggcggacaggtatccg
gtaagcggcagggtcggaacaggagagcgcacgagggagcttccagggggaaacgcctggtatc
tttatagtcctgtcgggtttcgccacctctgacttgagcgtcgatttttgtgatgctcgtcagg
ggggcggagcctatggaaaaacgccagcaacgcggcctttttacggttcctggccttttgctgg
ccttttgctcacatgt

Plasmid #1 (see FIG. 19 for a map of the plasmid)

(SEQ ID NO: 94)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTGGTACCGCCACCATGGGGTCTTCAAAATCTAAACCAAAGGACCC
CAGCCAGCGCGGCGGAGGTGGTTCTgacgcacaaacacgacgacgtgagcgtcgcgctgagaaa
caagctcaatggaaagctgcaaacGGCGGAGGTGGTTCTgattacaaggatgacgacgataagt
aaGAATTCTGCAGATATCGCTCGCTTTCTTGCTGTCCAATTTCTATTAAAGGTTCCTTTGTTCC
CTAAGCTCGACCAAAGGTTCCTTTGTGGCCCTGAAAAAGGGCCAAATTGGTGGCTGGTGTGGCT
AATGCCCTATGGCCCTGAAAAAGGGCCACTGGAGGATATTCATGCACTCGACCAAAGGTTCCTT
TGTGGCCCTGAAAAAGGGCCAAATTGGTGGCTGGTGTGGCTAATGCCCTATGGCCCTGAAAAAG
GGCCACTGGAGGATATTCATGCACTCGAGTACTAAACTGGGGGATATTATGAAGGGCCTTGAGC
ATCTGGATTCTGCCTAATTCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGCTTCTGAGGCGGAAAGAACCAGCTGGGGCTCTAGGGGGTA
TCCCCACGCGCCCTGTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCTTTCTCGCCACGT
TCGCCGGCTTTCCCCGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTGGGCCATCGCCCTGA
TAGACGGTTTTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTTGCCGATTTC
GGCCTATTGGTTAAAAAATGAGCTGATTTAACAAAAATTTAACGCGAATTAATTCTGTGGAATG
TGTGTCAGTTAGGGTGTGGAAAGTCCCCAGGCTCCCCAGCAGGCAGAAGTATGCAAAGCATGCA
TCTCAATTAGTCAGCAACCAGGTGTGGAAAGTCCCCAGGCTCCCCAGCAGGCAGAAGTATGCAA
AGCATGCATCTCAATTAGTCAGCAACCATAGTCCCGCCCCTAACTCCGCCCATCCCGCCCCTAA
CTCCGCCCAGTTCCGCCCATTCTCCGCCCCATGGCTGACTAATTTTTTTTATTTATGCAGAGGC
CGAGGCCGCCTCTGCCTCTGAGCTATTCCAGAAGTAGTGAGGAGGCTTTTTTGGAGGCCTAGGC
TTTTGCAAAAAGCTCCCGGGAGCTTGTATATCCATTTTCGGATCTGATCAAGAGACAGGATGAG
GATCGTTTCGCATGATTGAACAAGATGGATTGCACGCAGGTTCTCCGGCCGCTTGGGTGGAGAG
GCTATTCGGCTATGACTGGGCACAACAGACAATCGGCTGCTCTGATGCCGCCGTGTTCCGGCTG
TCAGCGCAGGGGCGCCCGGTTCTTTTTGTCAAGACCGACCTGTCCGGTGCCCTGAATGAACTGC
AGGACGAGGCAGCGCGGCTATCGTGGCTGGCCACGACGGGCGTTCCTTGCGCAGCTGTGCTCGA
CGTTGTCACTGAAGCGGGAAGGGACTGGCTGCTATTGGGCGAAGTGCCGGGGCAGGATCTCCTG
TCATCTCACCTTGCTCCTGCCGAGAAAGTATCCATCATGGCTGATGCAATGCGGCGGCTGCATA
CGCTTGATCCGGCTACCTGCCCATTCGACCACCAAGCGAAACATCGCATCGAGCGAGCACGTAC
TCGGATGGAAGCCGGTCTTGTCGATCAGGATGATCTGGACGAAGAGCATCAGGGGCTCGCGCCA
GCCGAACTGTTCGCCAGGCTCAAGGCGCGCATGCCCGACGGCGAGGATCTCGTCGTGACCCATG
GCGATGCCTGCTTGCCGAATATCATGGTGGAAAATGGCCGCTTTTCTGGATTCATCGACTGTGG
CCGGCTGGGTGTGGCGGACCGCTATCAGGACATAGCGTTGGCTACCCGTGATATTGCTGAAGAG
CTTGGCGGCGAATGGGCTGACCGCTTCCTCGTGCTTTACGGTATCGCCGCTCCCGATTCGCAGC
GCATCGCCTTCTATCGCCTTCTTGACGAGTTCTTCTGAGCGGGACTCTGGGGTTCGAAATGACC
GACCAAGCGACGCCCAACCTGCCATCACGAGATTTCGATTCCACCGCCGCCTTCTATGAAAGGT
TGGGCTTCGGAATCGTTTTCCGGGACGCCGGCTGGATGATCCTCCAGCGCGGGGATCTCATGCT
GGAGTTCTTCGCCCACCCCAACTTGTTTATTGCAGCTTATAATGGTTACAAATAAAGCAATAGC
ATCACAAATTTCACAAATAAAGCATTTTTTTCACTGCATTCTAGTTGTGGTTTGTCCAAACTCA
TCAATGTATCTTATCATGTCTGTATACCGTCGACCTCTAGCTAGAGCTTGGCGTAATCATGGTC
ATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAACGCGCGGG
GAGAGGCGGTTTGCGTATTGGGCGCTCTTCCGCTTCCTCGCTCACTGACTCGCTGCGCTCGGTC
GTTCGGCTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGGG
AAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTAACAGGAT
TAGCAGAGCGAGGTATGTAGGCGGTGCTACAGAGTTCTTGAAGTGGTGGCCTAACTACGGCTAC
ACTAGAAGAACAGTATTTGGTATCTGCGCTCTGCTGAAGCCAGTTACCTTCGGAAAAAGAGTTG
GTAGCTCTTGATCCGGCAAACAAACCACCGCTGGTAGCGGTTTTTTTGTTTGCAAGCAGCAGAT
TACGCGCAGAAAAAAAGGATCTCAAGAAGATCCTTTGATCTTTTCTACGGGGTCTGACGCTCAG
TGGAACGAAAACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATA
GTTGCCTGACTCCCCGTCGTGTAGATAACTACGATACGGGAGGGCTTACCATCTGGCCCCAGTG
CTGCAATGATACCGCGAGACCCACGCTCACCGGCTCCAGATTTATCAGCAATAAACCAGCCAGC
CGGAAGGGCCGAGCGCAGAAGTGGTCCTGCAACTTTATCCGCCTCCATCCAGTCTATTAATTGT
TGCCGGGAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTC
TTACTGTCATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGAAAACTCTCAAGGA
TCTTACCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAAAGGGA
ATAAGGGCGACACGGAAATGTTGAATACTCATACTCTTCCTTTTTCAATATTATTGAAGCATTT
ATCAGGGTTATTGTCTCATGAGCGGATACATATTTGAATGTATTTAGAAAAATAAACAAATAGG
GGTTCCGCGCACATTTCCCCGAAAAGTGCCACCTGACGTC

Plasmid #2 (see FIG. 20 for a map of the plasmid)

(SEQ ID NO: 95)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTGGTACCGCCACCAtgctgtgctgcatcagaagaactaaaccggt
tgagaagaatgaagaggccgatcaggagctgcagtcgacggtgccgcgggcccgggatccaccg
gtcgccaccgacgcacaaacacgacgacgtgagcgtcgcgctgagaaacaagctcaatggaaag
ctgcaaacGGCGGAGGTGGTTCTgattacaaggatgacgacgataagtaaGAATTCTGCAGATA
TCGCTCGCTTTCTTGCTGTCCAATTTCTATTAAAGGTTCCTTTGTTCCCTAAGCTCGACCAAAG
GTTCCTTTGTGGCCCTGAAAAAGGGCCAAATTGGTGGCTGGTGTGGCTAATGCCCTATGGCCCT
GAAAAAGGGCCACTGGAGGATATTCATGCACTCGACCAAAGGTTCCTTTGTGGCCCTGAAAAAG
GGCCAAATTGGTGGCTGGTGTGGCTAATGCCCTATGGCCCTGAAAAAGGGCCACTGGAGGATAT
TCATGCACTCGAGTACTAAACTGGGGGATATTATGAAGGGCCTTGAGCATCTGGATTCTGCCTA
ATTCTAGAGGGCCCGTTTAAACCCGCTGATCAGCCTCGACTGTGCCTTCTAGTTGCCAGCCATC
TGTTGTTTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGGGGTGGGG
TGGGGCAGGACAGCAAGGGGGAGGATTGGGAAGACAATAGCAGGCATGCTGGGGATGCGGTGGG
CTCTATGGCTTCTGAGGCGGAAAGAACCAGCTGGGGCTCTAGGGGGTATCCCCACGCGCCCTGT
AGCGGCGCATTAAGCGCGGCGGGTGTGGTGGTTACGCGCAGCGTGACCGCTACACTTGCCAGCG
CCCTAGCGCCCGCTCCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTACGGCACCTCGACCCC
AAAAAACTTGATTAGGGTGATGGTTCACGTAGTGGGCCATCGCCCTGATAGACGGTTTTTCGCC
CTTTGACGTTGGAGTCCACGTTCTTTAATAGTGGACTCTTGTTCCAAACTGGAACAACACTCAA
CCCTATCTCGGTCTATTCTTTTGATTTATAAGGGATTTTGCCGATTTCGGCCTATTGGTTAAAA
AATGAGCTGATTTAACAAAAATTTAACGCGAATTAATTCTGTGGAATGTGTGTCAGTTAGGGTG
TGGAAAGTCCCCAGGCTCCCCAGCAGGCAGAAGTATGCAAAGCATGCATCTCAATTAGTCAGCA
ACCAGGTGTGGAAAGTCCCCAGGCTCCCCAGCAGGCAGAAGTATGCAAAGCATGCATCTCAATT
AGTCAGCAACCATAGTCCCGCCCCTAACTCCGCCCATCCCGCCCCTAACTCCGCCCAGTTCCGC
CCATTCTCCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAAAAGCTCC
CGGGAGCTTGTATATCCATTTTCGGATCTGATCAAGAGACAGGATGAGGATCGTTTCGCATGAT
TGAACAAGATGGATTGCACGCAGGTTCTCCGGCCGCTTGGGTGGAGAGGCTATTCGGCTATGAC
TGGGCACAACAGACAATCGGCTGCTCTGATGCCGCCGTGTTCCGGCTGTCAGCGCAGGGGCGCC
CGGTTCTTTTTGTCAAGACCGACCTGTCCGGTGCCCTGAATGAACTGCAGGACGAGGCAGCGCG
GCTATCGTGGCTGGCCACGACGGGCGTTCCTTGCGCAGCTGTGCTCGACGTTGTCACTGAAGCG
GGAAGGGACTGGCTGCTATTGGGCGAAGTGCCGGGGCAGGATCTCCTGTCATCTCACCTTGCTC
CTGCCGAGAAAGTATCCATCATGGCTGATGCAATGCGGCGGCTGCATACGCTTGATCCGGCTAC
CTGCCCATTCGACCACCAAGCGAAACATCGCATCGAGCGAGCACGTACTCGGATGGAAGCCGGT
CTTGTCGATCAGGATGATCTGGACGAAGAGCATCAGGGGCTCGCGCCAGCCGAACTGTTCGCCA
GGCTCAAGGCGCGCATGCCCGACGGCGAGGATCTCGTCGTGACCCATGGCGATGCCTGCTTGCC
GAATATCATGGTGGAAAATGGCCGCTTTTCTGGATTCATCGACTGTGGCCGGCTGGGTGTGGCG
GACCGCTATCAGGACATAGCGTTGGCTACCCGTGATATTGCTGAAGAGCTTGGCGGCGAATGGG
CTGACCGCTTCCTCGTGCTTTACGGTATCGCCGCTCCCGATTCGCAGCGCATCGCCTTCTATCG
CCTTCTTGACGAGTTCTTCTGAGCGGGACTCTGGGGTTCGAAATGACCGACCAAGCGACGCCCA
ACCTGCCATCACGAGATTTCGATTCCACCGCCGCCTTCTATGAAAGGTTGGGCTTCGGAATCGT
TTTCCGGGACGCCGGCTGGATGATCCTCCAGCGCGGGGATCTCATGCTGGAGTTCTTCGCCCAC
CCCAACTTGTTTATTGCAGCTTATAATGGTTACAAATAAAGCAATAGCATCACAAATTTCACAA
ATAAAGCATTTTTTTCACTGCATTCTAGTTGTGGTTTGTCCAAACTCATCAATGTATCTTATCA
TGTCTGTATACCGTCGACCTCTAGCTAGAGCTTGGCGTAATCATGGTCATAGCTGTTTCCTGTG
TGAAATTGTTATCCGCTCACAATTCCACACAACATACGAGCCGGAAGCATAAAGTGTAAAGCCT
GGGGTGCCTAATGAGTGAGCTAACTCACATTAATTGCGTTGCGCTCACTGCCCGCTTTCCAGTC
GGGAAACCTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGCGGCGAG
CGGTATCAGCTCACTCAAAGGCGGTAATACGGTTATCCACAGAATCAGGGGATAACGCAGGAAA
GAACATGTGAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGATCCGG
CAAACAAACCACCGCTGGTAGCGGTTTTTTTGTTTGCAAGCAGCAGATTACGCGCAGAAAAAAA
GGATCTCAAGAAGATCCTTTGATCTTTTCTACGGGGTCTGACGCTCAGTGGAACGAAAACTCAC
GTTAAGGGATTTTGGTCATGAGATTATCAAAAAGGATCTTCACCTAGATCCTTTTAAATTAAAA
ATGAAGTTTTAAATCAATCTAAAGTATATATGAGTAAACTTGGTCTGACAGTTACCAATGCTTA
ATCAGTGAGGCACCTATCTCAGCGATCTGTCTATTTCGTTCATCCATAGTTGCCTGACTCCCCG
TCGTGTAGATAACTACGATACGGGAGGGCTTACCATCTGGCCCCAGTGCTGCAATGATACCGCG
AGACCCACGCTCACCGGCTCCAGATTTATCAGCAATAAACCAGCCAGCCGGAAGGGCCGAGCGC
AGAAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGAGTTACATGA
TCCCCCATGTTGTGCAAAAAAGCGGTTAGCTCCTTCGGTCCTCCGATCGTTGTCAGAAGTAAGT
TGGCCGCAGTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACATTT
CCCCGAAAAGTGCCACCTGACGTC

Plasmid #3 (see FIG. 21 for a map of the plasmid)

(SEQ ID NO: 96)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTGGTACCGCCACCatggattacaaggatgacgacgataagGGCGG
AGGTGGTTCTgacgcacaaacacgacgacgtgagcgtcgcgctgagaaacaagctcaatggaaa
gctgcaaacGGCGGAGGTGGTTCTaagctgaaccctcctgatgagagtggccccggctgcatga
gctgctgtgtgctctcctaaGAATTCTGCAGATATCGCTCGCTTTCTTGCTGTCCAATTTCTAT
TAAAGGTTCCTTTGTTCCCTAAGCTCGACCAAAGGTTCCTTTGTGGCCCTGAAAAAGGGCCAAA
TTGGTGGCTGGTGTGGCTAATGCCCTATGGCCCTGAAAAAGGGCCACTGGAGGATATTCATGCA
CTCGACCAAAGGTTCCTTTGTGGCCCTGAAAAAGGGCCAAATTGGTGGCTGGTGTGGCTAATGC
CCTATGGCCCTGAAAAAGGGCCACTGGAGGATATTCATGCACTCGAGTACTAAACTGGGGGATA
TTATGAAGGGCCTTGAGCATCTGGATTCTGCCTAATTCTAGAGGGCCCGTTTAAACCCGCTGAT
CAGCCTCGACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGCATTGT
CTGAGTAGGTGTCATTCTATTCTGGGGGGTGGGGTGGGGCAGGACAGCAAGGGGGAGGATTGGG
AAGACAATAGCAGGCATGCTGGGGATGCGGTGGGCTCTATGGCTTCTGAGGCGGAAAGAACCAG
CTGGGGCTCTAGGGGGTATCCCCACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCGCTTTCTTCC
CTTCCTTTCTCGCCACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTAGGGTGATGGTTCACGT
AGTGGGCCATCGCCCTGATAGACGGTTTTTCGCCCTTTGACGTTGGAGTCCACGTTCTTTAATA
GTGGACTCTTGTTCCAAACTGGAACAACACTCAACCCTATCTCGGTCTATTCTTTTGATTTATA
AGGGATTTTGCCGATTTCGGCCTATTGGTTAAAAAATGAGCTGATTTAACAAAAATTTAACGCG
AATTAATTCTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAGGCTCCCCA
GCAGGCAGAAGTATGCAAAGCATGCATCTCAATTAGTCAGCAACCATAGTCCCGCCCCTAACTC
CGCCCATCCCGCCCCTAACTCCGCCCAGTTCCGCCCATTCTCCGCCCCATGGCTGACTAATTTT
TTTTATTTATGCAGAGGCCGAGGCCGCCTCTGCCTCTGAGCTATTCCAGAAGTAGTGAGGAGGC
TTTTTTGGAGGCCTAGGCTTTTGCAAAAAGCTCCCGGGAGCTTGTATATCCATTTTCGGATCTG
ATCAAGAGACAGGATGAGGATCGTTTCGCATGATTGAACAAGATGGATTGCACGCAGGTTCTCC
GGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCG
GTGCCCTGAATGAACTGCAGGACGAGGCAGCGCGGCTATCGTGGCTGGCCACGACGGGCGTTCC
TTGCGCAGCTGTGCTCGACGTTGTCACTGAAGCGGGAAGGGACTGGCTGCTATTGGGCGAAGTG
CCGGGGCAGGATCTCCTGTCATCTCACCTTGCTCCTGCCGAGAAAGTATCCATCATGGCTGATG
CAATGCGGCGGCTGCATACGCTTGATCCGGCTACCTGCCCATTCGACCACCAAGCGAAACATCG
CATCGAGCGAGCACGTACTCGGATGGAAGCCGGTCTTGTCGATCAGGATGATCTGGACGAAGAG
CATCAGGGGCTCGCGCCAGCCGAACTGTTCGCCAGGCTCAAGGCGCGCATGCCCGACGGCGAGG
ATCTCGTCGTGACCCATGGCGATGCCTGCTTGCCGAATATCATGGTGGAAAATGGCCGCTTTTC
TGGATTCATCGACTGTGGCCGGCTGGGTGTGGCGGACCGCTATCAGGACATAGCGTTGGCTACC
CGTGATATTGCTGAAGAGCTTGGCGGCGAATGGGCTGACCGCTTCCTCGTGCTTTACGGTATCG
CCGCTCCCGATTCGCAGCGCATCGCCTTCTATCGCCTTCTTGACGAGTTCTTCTGAGCGGGACT
CTGGGGTTCGAAATGACCGACCAAGCGACGCCCAACCTGCCATCACGAGATTTCGATTCCACCG
CCGCCTTCTATGAAAGGTTGGGCTTCGGAATCGTTTTCCGGGACGCCGGCTGGATGATCCTCCA
GCGCGGGGATCTCATGCTGGAGTTCTTCGCCCACCCCAACTTGTTTATTGCAGCTTATAATGGT
TACAAATAAAGCAATAGCATCACAAATTTCACAAATAAAGCATTTTTTTCACTGCATTCTAGTT
GTGGTTTGTCCAAACTCATCAATGTATCTTATCATGTCTGTATACCGTCGACCTCTAGCTAGAG
CTTGGCGTAATCATGGTCATAGCTGTTTCCTGTGTGAAATTGTTATCCGCTCACAATTCCACAC
AACATACGAGCCGGAAGCATAAAGTGTAAAGCCTGGGGTGCCTAATGAGTGAGCTAACTCACAT
TAATTGCGTTGCGCTCACTGCCCGCTTTCCAGTCGGGAAACCTGTCGTGCCAGCTGCATTAATG
AATCGGCCAACGCGCGGGGAGAGGCGGTTTGCGTATTGGGCGCTCTTCCGCTTCCTCGCTCACT
GACTCGCTGCGCTCGGTCGTTCGGCTGCGGCGAGCGGTATCAGCTCACTCAAAGGCGGTAATAC
GGTTATCCACAGAATCAGGGGATAACGCAGGAAAGAACATGTGAGCAAAAGGCCAGCAAAAGGC
CAGGAACCGTAAAAAGGCCGCGTTGCTGGCGTTTTTCCATAGGCTCCGCCCCCCTGACGAGCAT
CACAAAAATCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATACCTGTC
CGCCTTTCTCCCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCCGACCGCTGCG
CCTTATCCGGTAACTATCGTCTTGAGTCCAACCCGGTAAGACACGACTTATCGCCACTGGCAGC
AGCCACTGGTAACAGGATTAGCAGAGCGAGGTATGTAGGCGGTGCTACAGAGTTCTTGAAGTGG
TGGCCTAACTACGGCTACACTAGAAGAACAGTATTTGGTATCTGCGCTCTGCTGAAGCCAGTTA
CCTTCGGAAAAAGAGTTGGTAGCTCTTGATCCGGCAAACAAACCACCGCTGGTAGCGGTTTTTT
TGTTTGCAAGCAGCAGATTACGCGCAGAAAAAAAGGATCTCAAGAAGATCCTTTGATCTTTTCT
ACGGGGTCTGACGCTCAGTGGAACGAAAACTCACGTTAAGGGATTTTGGTCATGAGATTATCAA
AAAGGATCTTCACCTAGATCCTTTTAAATTAAAAATGAAGTTTTAAATCAATCTAAAGTATATA
TGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCTCCAGATTTATC
AGCAATAAACCAGCCAGCCGGAAGGGCCGAGCGCAGAAGTGGTCCTGCAACTTTATCCGCCTCC
ATCCAGTCTATTAATTGTTGCCGGGAAGCTAGAGTAAGTAGTTCGCCAGTTAATAGTTTGCGCA
ACGTTGTTGCCATTGCTACAGGCATCGTGGTGTCACGCTCGTCGTTTGGTATGGCTTCATTCAG
CTCCGGTTCCCAACGATCAAGGCGAGTTACATGATCCCCCATGTTGTGCAAAAAAGCGGTTAGC
TCCTTCGGTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAATA
CGGGATAATACCGCGCCACATAGCAGAACTTTAAAAGTGCTCATCATTGGAAAACGTTCTTCGG
GGCGAAAACTCTCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATACTCTTCCTTTTTC
AATATTATTGAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGACGTC

Plasmid #4 (see FIG. 22 for a map of the plasmid)

(SEQ ID NO: 97)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTGGTACCGAGCTCGGATCCGCCACCatggattacaaggatgacga
cgataagGGCGGAGGTGGTTCTgcttctaactttactcagttcgttctcgtcgacaatggcgga
actggcgacgtgactgtcgccccaagcaacttcgctaacggggtcgctgaatggatcagctcta
actcgcgttcacaggcttacaaagtaacctgtagcgttcgtcagagctctgcgcagaatcgcaa
atacaccatcaaagtcgaggtgcctaaaggcgcatggcgttcgtacttaaatatggaactaacc
attccaattttctccacgaactccgactgcgagcttattgttaaggcaatgcaaggtctcctaa
aagatggaaacccgattccctcagcaatcgcagcaaactccggcatctacGGCGGAGGTGGTTC
TaagctgaaccctcctgatgagagtggccccggctgcatgagctgctgtgtgctctcctaaGAA
TTCTGCAGATATCCCTAAGGTACCTAATTGCCTAGAAAACATGAGGATCACCCATGTCTGCAGG
TCGACTCTAGAAAACATGAGGATCACCCATGTCTGCAGTATTCCCGGGTTCATTAGATCCTAAG
GTACCTAATTGCCTAGAAAACATGAGGATCACCCATGTCTGCAGGTCGACTCTAGAAAACATGA
GGATCACCCATGTCTGCAGTATTCCCGGGTTCATTAGACTCGAGTCTAGAGGGCCCGTTTAAAC
CCGCTGATCAGCCTCGACTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAATGAGGAAATTGCAT
CGCATTGTCTGAGTAGGTGTCATTCTATTCTGGGGGGTGGGGTGGGGCAGGACAGCAAGGGGGA
GGATTGGGAAGACAATAGCAGGCATGCTGGGGATGCGGTGGGCTCTATGGCTTCTGAGGCGGAA
AGAACCAGCTGGGGCTCTAGGGGGTATCCCCACGCGCCCTGTAGCGGCGCATTAAGCGCGGCGG
GTGTGGTGGTTACGCGCAGCGTGACCGCTACACTTGCCAGCGCCCTAGCGCCCGCTCCTTTCGC
TTTCTTCCCTTCCTTTCTCGCCACGTTCGCCGGCTTTCCCCGTCAAGCTCTAAATCGGGGGCTC
CCTTTAGGGTTCCGATTTAGTGCTTTACGGCACCTCGACCCCAAAAAACTTGATTAGGGTGATG
GTTCACGTAGTGGGCCATCGCCCTGATAGACGGTTTTTCGCCCTTTGACGTTGGAGTCCACGTT
CTTTAATAGTGGACTCTTGTTCCAAACTGGAACAACACTCAACCCTATCTCGGTCTATTCTTTT
GATTTATAAGGGATTTTGCCGATTTCGGCCTATTGGTTAAAAAATGAGCTGATTTAACAAAAAT
TTAACGCGAATTAATTCTGTGGAATGTGTGTCAGTTAGGGTGTGGAAAGTCCCCAGGCTCCCCA
GCAGGCAGAAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATAGTCCCGCC
CCTAACTCCGCCCATCCCGCCCCTAACTCCGCCCAGTTCCGCCCATTCTCCGCCCCATGGCTGA
CTAATTTTTTTTATTTATGCAGAGGCCGAGGCCGCCTCTGCCTCTGAGCTATTCCAGAAGTAGT
GAGGAGGCTTTTTTGGAGGCCTAGGCTTTTGCAAAAAGCTCCCGGGAGCTTGTATATCCATTTT
CGGATCTGATCAAGAGACAGGATGAGGATCGTTTCGCATGATTGAACAAGATGGATTGCACGCA
GGTTCTCCGGCCGCTTGGGTGGAGAGGCTATTCGGCTATGACTGGGCACAACAGACAATCGGCT
GCTCTGATGCCGCCGTGTTCCGGCTGTCAGCGCAGGGGCGCCCGGTTCTTTTTGTCAAGACCGA
CCTGTCCGGTGCCCTGAATGAACTGCAGGACGAGGCAGCGCGGCTATCGTGGCTGGCCACGACG
GGCGTTCCTTGCGCAGCTGTGCTCGACGTTGTCACTGAAGCGGGAAGGGACTGGCTGCTATTGG
GCGAAGTGCCGGGGCAGGATCTCCTGTCATCTCACCTTGCTCCTGCCGAGAAAGTATCCATCAT
GGCTGATGCAATGCGGCGGCTGCATACGCTTGATCCGGCTACCTGCCCATTCGACCACCAAGCG
AAACATCGCATCGAGCGAGCACGTACTCGGATGGAAGCCGGTCTTGTCGATCAGGATGATCTGG
ACGAAGAGCATCAGGGGCTCGCGCCAGCCGAACTGTTCGCCAGGCTCAAGGCGCGCATGCCCGA
CGGCGAGGATCTCGTCGTGACCCATGGCGATGCCTGCTTGCCGAATATCATGGTGGAAAATGGC
CGCTTTTCTGGATTCATCGACTGTGGCCGGCTGGGTGTGGCGGACCGCTATCAGGACATAGCGT
TGGCTACCCGTGATATTGCTGAAGAGCTTGGCGGCGAATGGGCTGACCGCTTCCTCGTGCTTTA
CGGTATCGCCGCTCCCGATTCGCAGCGCATCGCCTTCTATCGCCTTCTTGACGAGTTCTTCTGA
GCGGGACTCTGGGGTTCGAAATGACCGACCAAGCGACGCCCAACCTGCCATCACGAGATTTCGA
TTCCACCGCCGCCTTCTATGAAAGGTTGGGCTTCGGAATCGTTTTCCGGGACGCCGGCTGGATG
ATCCTCCAGCGCGGGGATCTCATGCTGGAGTTCTTCGCCCACCCCAACTTGTTTATTGCAGCTT
ATAATGGTTACAAATAAAGCAATAGCATCACAAATTTCACAAATAAAGCATTTTTTTCACTGCA
TTCTAGTTGTGGTTTGTCCAAACTCATCAATGTATCTTATCATGTCTGTATACCGTCGACCTCT
AGCTAGAGCTTGGCGTAATCATGGTCATAGCTGTTTCCTGTGTGAAATTGTTATCCGCTCACAA
TTCCACACAACATACGAGCCGGAAGCATAAAGTGTAAAGCCTGGGGTGCCTAATGAGTGAGCTA
ACTCACATTAATTGCGTTGCGCTCACTGCCCGCTTTCCAGTCGGGAAACCTGTCGTGCCAGCTG
CATTAATGAATCGGCCAACGCGCGGGGAGAGGCGGTTTGCGTATTGGGCGCTCTTCCGCTTCCT
CGCTCACTGACTCGCTGCGCTCGGTCGTTCGGCTGCGGCGAGCGGTATCAGCTCACTCAAAGGC
GGTAATACGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCCCCCCTG
ACGAGCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTACCGGA
TACCTGTCCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTTCAGCCCGA
CCGCTGCGCCTTATCCGGTAACTATCGTCTTGAGTCCAACCCGGTAAGACACGACTTATCGCCA
CTGGCAGCAGCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCTCTGCTGAA
GCCAGTTACCTTCGGAAAAAGAGTTGGTAGCTCTTGATCCGGCAAACAAACCACCGCTGGTAGC
GGTTTTTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAG
CGATCTGTCTATTTCGTTCATCCATAGTTGCCTGACTCCCCGTCGTGTAGATAACTACGATACG
GGAGGGCTTACCATCTGGCCCCAGTGCTGCAATGATACCGCGAGACCCACGCTCACCGGCTCCA
GATTTATCAGCAATAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTTCGCCAGTTAATAG
TTTGCGCAACGTTGTTGCCATTGCTACAGGCATCGTGGTGTCACGCTCGTCGTTTGGTATGGCT
TCATTCAGCTCCGGTTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCCGG
CGTCAATACGGGATAATACCGCGCCACATAGCAGAACTTTAAAAGTGCTCATCATTGGAAAACG
TTCTTCGGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAAAACAG
GAAGGCAAAATGCCGCAAAAAAGGGAATAAGGGCGACACGGAAATGTTGAATACTCATACTCTT
CCTTTTTCAATATTATTGAAGCATTTATCAGGGTTATTGTCTCATGAGCGGATACATATTTGAA
TGTATTTAGAAAAATAAACAAATAGGGGTTCCGCGCACATTTCCCCGAAAAGTGCCACCTGACG
TC

Plasmid #5 (see FIG. 23 for a map of the plasmid)

(SEQ ID NO: 98)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTGGTACCGAGCTCGGATCCGCCACCatggattacaaggatgacga
cgataagGGCGGAGGTGGTTCTtccaaaaccatcgttctttcggtcggcgaggctactcgcact
ctgactgagatccagtccaccgcagaccgtcagatcttcgaagagaaggtcgggcctctggtgg
gtcggctgcgcctcacggcttcgctccgtcaaaacggagccaagaccgcgtatcgcgtcaacct
aaaactggatcaggcggacgtcgttgattgctccaccagcgtctgcggcgagcttccgaaagtg
cgctacactcaggtatggtcgcacgacgtgacaatcgttgcgaatagcaccgaggcctcgcgca
aatcgttgtacgatttgaccaagtccctcgtcgcgacctcgcaggtcgaagatcttgtcgtcaa
ccttgtgccgctgggccgtGGCGGAGGTGGTTCTaagctgaaccctcctgatgagagtggcccc
ggctgcatgagctgctgtgtgctctcctaaGAATTCTGCAGATATCGATCCTAAGGTACCTAAT
TGCCTAGAAAGGAGCAGACGATATGGCGTCGCTCCCTGCAGGTCGACTCTAGAAACCAGCAGAG
CATATGGGCTCGCTGGCTGCAGTATTCCCGGGTTCATTAGATCCTAAGGTACCTAATTGCCTAG
AAAGGAGCAGACGATATGGCGTCGCTCCCTGCAGGTCGACTCTAGAAACCAGCAGAGCATATGG
GCTCGCTGGCTGCAGTATTCCCGGGTTCATTAGATCTCGAGTCTAGAGGGCCCGTTTAAACCCG
CTGATCAGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGA
TTGGGAAGACAATAGCAGGCATGCTGGGGATGCGGTGGGCTCTATGGCTTCTGAGGCGGAAAGA
ACCAGCTGGGGCTCTAGGGGGTATCCCCACGCGCCCTGTAGCGGCGCATTAAGCGCGGCGGGTG
TGGTGGTTACGCGCAGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAAATCGGGGGCTCCCT
TTAGGGTTCCGATTTAGTGCTTTACGGCACCTCGACCCCAAAAAACTTGATTAGGGTGATGGTT
CACGTAGTGGGCCATCGCCCTGATAGACGGTTTTTCGCCCTTTGACGTTGGAGTCCACGTTCTT
TAATAGTGGACTCTTGTTCCAAACTGGAACAACACTCAACCCTATCTCGGTCTATTCTTTTGAT
TTATAAGGGATTTTGCCGATTTCGGCCTATTGGTTAAAAAATGAGCTGATTTAACAAAAATTTA
ACGCGAATTAATTCTGTGGAATGTGTGTCAGTTAGGGTGTGGAAAGTCCCCAGGCTCCCCAGCA
GGCAGAAGTATGCAAAGCATGCATCTCAATTAGTCAGCAACCAGGTGTGGAAAGTCCCCAGGCT
CCCCAGCAGGCAGAAGTATGCAAAGCATGCATCTCAATTAGTCAGCAACCATAGTCCCGCCCCT
AACTCCGCCCATCCCGCCCCTAACTCCGCCCAGTTCCGCCCATTCTCCGCCCCATGGCTGACTA
ATTTTTTTTATTTATGCAGAGGCCGAGGCCGCCTCTGCCTCTGAGCTATTCCAGAAGTAGTGAG
GAGGCTTTTTTGGAGGCCTAGGCTTTTGCAAAAAGCTCCCGGGAGCTTGTATATCCATTTTCGG
ATCTGATCAAGAGACAGGATGAGGATCGTTTCGCATGATTGAACAAGATGGATTGCACGCAGGT
TCTCCGGCCGCTTGGGTGGAGAGGCTATTCGGCTATGACTGGGCACAACAGACAATCGGCTGCT
CTGATGCCGCCGTGTTCCGGCTGTCAGCGCAGGGGCGCCCGGTTCTTTTTGTCAAGACCGACCT
GTCCGGTGCCCTGAATGAACTGCAGGACGAGGCAGCGCGGCTATCGTGGCTGGCCACGACGGGC
GTTCCTTGCGCAGCTGTGCTCGACGTTGTCACTGAAGCGGGAAGGGACTGGCTGCTATTGGGCG
AAGTGCCGGGGCAGGATCTCCTGTCATCTCACCTTGCTCCTGCCGAGAAAGTATCCATCATGGC
TGATGCAATGCGGCGGCTGCATACGCTTGATCCGGCTACCTGCCCATTCGACCACCAAGCGAAA
CATCGCATCGAGCGAGCACGTACTCGGATGGAAGCCGGTCTTGTCGATCAGGATGATCTGGACG
AAGAGCATCAGGGGCTCGCGCCAGCCGAACTGTTCGCCAGGCTCAAGGCGCGCATGCCCGACGG
CGAGGATCTCGTCGTGACCCATGGCGATGCCTGCTTGCCGAATATCATGGTGGAAAATGGCCGC
TTTTCTGGATTCATCGACTGTGGCCGGCTGGGTGTGGCGGACCGCTATCAGGACATAGCGTTGG
CTACCCGTGATATTGCTGAAGAGCTTGGCGGCGAATGGGCTGACCGCTTCCTCGTGCTTTACGG
TATCGCCGCTCCCGATTCGCAGCGCATCGCCTTCTATCGCCTTCTTGACGAGTTCTTCTGAGCG
GGACTCTGGGGTTCGAAATGACCGACCAAGCGACGCCCAACCTGCCATCACGAGATTTCGATTC
CACCGCCGCCTTCTATGAAAGGTTGGGCTTCGGAATCGTTTTCCGGGACGCCGGCTGGATGATC
CTCCAGCGCGGGGATCTCATGCTGGAGTTCTTCGCCCACCCCAACTTGTTTATTGCAGCTTATA
ATGGTTACAAATAAAGCAATAGCATCACAAATTTCACAAATAAAGCATTTTTTTCACTGCATTC
TAGTTGTGGTTTGTCCAAACTCATCAATGTATCTTATCATGTCTGTATACCGTCGACCTCTAGC
TAGAGCTTGGCGTAATCATGGTCATAGCTGTTTCCTGTGTGAAATTGTTATCCGCTCACAATTC
CACACAACATACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGCCAGCTGCAT
TAATGAATCGGCCAACGCGCGGGGAGAGGCGGTTTGCGTATTGGGCGCTCTTCCGCTTCCTCGC
TCACTGACTCGCTGCGCTCGGTCGTTCGGCTGCGGCGAGCGGTATCAGCTCACTCAAAGGCGGT
AATACGGTTATCCACAGAATCAGGGGATAACGCAGGAAAGAACATGTGAGCAAAAGGCCAGCAA
AAGGCCAGGAACCGTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATCGCCACTG
GCAGCAGCCACTGGTAACAGGATTAGCAGAGCGAGGTATGTAGGCGGTGCTACAGAGTTCTTGA
AGTGGTGGCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTGGTAGCGGT
TTTTTTGTTTGCAAGCAGCAGATTACGCGCAGAAAAAAAGGATCTCAAGAAGATCCTTTGATCT
TTTCTACGGGGTCTGACGCTCAGTGGAACGAAAACTCACGTTAAGGGATTTTGGTCATGAGATT
ATCAAAAAGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATCTCAGCGA
TCTGTCTATTTCGTTCATCCATAGTTGCCTGACTCCCCGTCGTGTAGATAACTACGATACGGGA
GGGCTTACCATCTGGCCCCAGTGCTGCAATGATACCGCGAGACCCACGCTCACCGGCTCCAGAT
TTATCAGCAATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTTAATAGTTT
GCGCAACGTTGTTGCCATTGCTACAGGCATCGTGGTGTCACGCTCGTCGTTTGGTATGGCTTCA
TTCAGCTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTCATGGT
TATGGCAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCCCGGCGT
CAATACGGGATAATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGTAACCCACTCGT
GCACCCAACTGATCTTCAGCATCTTTTACTTTCACCAGCGTTTCTGGGTGAGCAAAAACAGGAA
GGCAAAATGCCGCAAAAAAGGGAATAAGGGCGACACGGAAATGTTGAATACTCATACTCTTCCT
TTTTCAATATTATTGAAGCATTTATCAGGGTTATTGTCTCATGAGCGGATACATATTTGAATGT
ATTTAGAAAAATAAACAAATAGGGGTTCCGCGCACATTTCCCCGAAAAGTGCCACCTGACGTC

Plasmid #6 (see FIG. 24 for a map of the plasmid)

(SEQ ID NO: 99)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgtgtctagactgcagagggccctgcgtatgagtgcaagtgggttttag
gaccaggatgaggcggggtgggggtgcctacctgacgaccgaccccgacccactggacaagcac
ccaacccccattccccaaattgcgcatcccctatcagagagggggaggggaaacaggatgcggc
gaggcgcgtgcgcactgccagcttcagcaccgcggacagtgccttcgcccccgcctggcggcgc
gcgccaccgccgcctcagcactgaaggcgcgctgacgtcactcgccggtcccccgcaaactccc
cttcccggccaccttggtcgcgtccgcgccgccgccggcccagccggaccgcaccacgcgaggc
gcgagataggggggcacgggcgcgaccatctgcgctgcggcgccggcgactcagcgctgcctca
gtctgcggtgggcagcggaggagtcgtgtcgtgcctgagagcgcagtcgagaaggtaccGCCAC
CatggattacaaggatgacgacgataagGGCGGAGGTGGTTCTgacgcacaaacacgacgacgt
gagcgtcgcgctgagaaacaagctcaatggaaagctgcaaacGGCGGAGGTGGTTCTaagctga
accctcctgatgagagtggccccggctgcatgagctgctgtgtgctctcctaagaattcgatat
caagcttatcgataatcaacctctggattacaaaatttgtgaaagattgactggtattcttaac
tatgttgctccttttacgctatgtggatacgctgctttaatgcctttgtatcatgctattgctt
cccgtatggctttcattttctcctccttgtataaatcctggttgctgtctctttatgaggagtt
gtggcccgttgtcaggcaacgtggcgtggtgtgcactgtgtttgctgacgcaacccccactggt
tggggcattgccaccacctgtcagctcctttccgggactttcgctttccccctccctattgcca
cggcggaactcatcgccgcctgccttgcccgctgctggacaggggctcggctgttgggcactga
caattccgtggtgttgtcggggaaatcatcgtcctttccttggctgctcgcctatgttgccacc
tggattctgcgcgggacgtccttctgctacgtcccttcggccctcaatccagcggaccttcctt
cccgcggcctgctgccggctctgcggcctcttccgcgtcttcgccttcgccctcagacgagtcg
gatctccctttgggccgcctccccgcatcgataccgagcgctgctcgagagatctacgggtggc
atccctgtgacccctccccagtgcctctcctggccctggaagttgccactccagtgcccaccag
ccttgtcctaataaaattaagttgcatcattttgtctgactaggtgtccttctataatattatg
gggtggaggggggtggtatggagcaaggggcaagttgggaagacaacctgtagggcctgcgggg
tctattgggaaccaagctggagtgcagtggcacaatcttggctcactgcaatctccgcctcctg
ggttcaagcgattctcctgcctcagcctcccgagttgttgggattccaggcatgcatgaccagg
ctcagctaatttttgtttttttggtagagacggggtttcaccatattggccaggctggtctcca
actcctaatctcaggtgatctacccaccttggcctcccaaattgctgggattacaggcgtgaac
cactgctcccttccctgtccttctgattttgtaggtaaccacgtgcggaccgagcggccgcagg
aacccctagtgatggagttggccactccctctctgcgcgctcgctcgctcactgaggccgggcg
accaaaggtcgcccgacgcccgggctttgcccgggcggcctcagtgagcgagcgagcgcgcagc
tgcctgcaggggcgcctgatgcggtattttctccttacgcatctgtgcggtatttcacaccgca
tacgtcaaagcaaccatagtacgcgccctgtagcggcgcattaagcgcggcgggtgtggtggtt
acgcgcagcgtgaccgctacacttgccagcgccttagcgcccgctcctttcgctttcttccctt
cctttctcgccacgttcgccggctttccccgtcaagctctaaatcgggggctccctttagggtt
ccgatttagtgctttacggcacctcgaccccaaaaaacttgatttgggtgatggttcacgtagt
gggccatcgccctgatagacggtttttcgccctttgacgttggagtccacgttctttaatagtg
gactcttgttccaaactggaacaacactcaactctatctcgggctattcttttgatttataagg
gattttgccgatttcggtctattggttaaaaaatgagctgatttaacaaaaatttaacgcgaat
tttaacaaaatattaacgtttacaattttatggtgcactctcagtacaatctgctctgatgccg
catagttaagccagccccgacacccgccaacacccgctgacgcgccctgacgggcttgtctgct
cccggcatccgcttacagacaagctgtgaccgtctccgggagctgcatgtgtcagaggttttca
ccgtcatcaccgaaacgcgcgagacgaaagggcctcgtgatacgcctatttttataggttaatg
tcatgataataatggtttcttagacgtcaggtggcacttttcggggaaatgtgcgcggaacccc
tatttgtttatttttctaaatacattcaaatatgtatccgctcatgagacaataaccctgataa
atgcttcaataatattgaaaaaggaagagtatgagtattcaacatttccgtgtcgcccttattc
ccttttttgcggcattttgccttcctgtttttgctcacccagaaacgctggtgaaagtaaaaga
tgctgaagatcagttgggtgcacgagtgggttacatcgaactggatctcaacagcggtaagatc
cttgagagttttcgccccgaagaacgttttccaatgatgagcacttttaaagttctgctatgtg
gcgcggtattatcccgtattgacgccgggcaagagcaactcggtcgccgcatacactattctca
gaatgacttggttgagtactcaccagtcacagaaaagcatcttacggatggcatgacagtaaga
gaattatgcagtgctgccataaccatgagtgataacactgcggccaacttacttctgacaacga
tcggaggaccgaaggagctaaccgcttttttgcacaacatgggggatcatgtaactcgccttga
tcgttgggaaccggagctgaatgaagccataccaaacgacgagcgtgacaccacgatgcctgta
gcaatggcaacaacgttgcgcaaactattaactggcgaactacttactctagcttcccggcaac
aattaatagactggatggaggcggataaagttgcaggaccacttctgcgctcggcccttccggc
tggctggtttattgctgataaatctggagccggtgagcgtgggtctcgcggtatcattgcagca
ctggggccagatggtaagccctcccgtatcgtagttatctacacgacggggagtcaggcaacta
tggatgaacgaaatagacagatcgctgagataggtgcctcactgattaagcattggtaactgtc
agaccaagtttactcatatatactttagattgatttaaaacttcatttttaatttaaaaggatc
taggtgaagatcctttttgataatctcatgaccaaaatcccttaacgtgagttttcgttccact
gagcgtcagaccccgtagaaaagatcaaaggatcttcttgagatcctttttttctgcgcgtaat
ctgctgcttgcaaacaaaaaaaccaccgctaccagcggtggtttgtttgccggatcaagagcta
ccaactctttttccgaaggtaactggcttcagcagagcgcagataccaaatactgttcttctag
tgtagccgtagttaggccaccacttcaagaactctgtagcaccgcctacatacctcgctctgct
aatcctgttaccagtggctgctgccagtggcgataagtcgtgtcttaccgggttggactcaaga
cgatagttaccggataaggcgcagcggtcgggctgaacggggggttcgtgcacacagcccagct
tggagcgaacgacctacaccgaactgagatacctacagcgtgagctatgagaaagcgccacgct
tcccgaagggagaaaggcggacaggtatccggtaagcggcagggtcggaacaggagagcgcacg
agggagcttccagggggaaacgcctggtatctttatagtcctgtcgggtttcgccacctctgac
ttgagcgtcgatttttgtgatgctcgtcaggggggcggagcctatggaaaaacgccagcaacgc
ggcctttttacggttcctggccttttgctggccttttgctcacatgt

Plasmid #7 (see FIG. 25 for a map of the plasmid)

(SEQ ID NO: 100)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgtgtctagactgcagagggccctgcgtatgagtgcaagtgggttttag
gaccaggatgaggcggggtgggggtgcctacctgacgaccgaccccgacccactggacaagcac
ccaacccccattccccaaattgcgcatcccctatcagagagggggaggggaaacaggatgcggc
gaggcgcgtgcgcactgccagcttcagcaccgcggacagtgccttcgcccccgcctggcggcgc
gcgccaccgccgcctcagcactgaaggcgcgctgacgtcactcgccggtcccccgcaaactccc
cttcccggccaccttggtcgcgtccgcgccgccgccggcccagccggaccgcaccacgcgaggc
gcgagataggggggcacgggcgcgaccatctgcgctgcggcgccggcgactcagcgctgcctca
gtctgcggtgggcagcggaggagtcgtgtcgtgcctgagagcgcagtcgagaaggtaccGGATC
CGCCACCatggattacaaggatgacgacgataagGGCGGAGGTGGTTCTgcttctaactttact
cagttcgttctcgtcgacaatggcggaactggcgacgtgactgtcgccccaagcaacttcgcta
acggggtcgctgaatggatcagctctaactcgcgttcacaggcttacaaagtaacctgtagcgt
tcgtcagagctctgcgcagaatcgcaaatacaccatcaaagtcgaggtgcctaaaggcgcatgg
cgttcgtacttaaatatggaactaaccattccaattttctccacgaactccgactgcgagctta
ttgttaaggcaatgcaaggtctcctaaaagatggaaacccgattccctcagcaatcgcagcaaa
ctccggcatctacGGCGGAGGTGGTTCTaagctgaaccctcctgatgagagtggccccggctgc
atgagctgctgtgtgctctcctaagaattcgatatcaagcttatcgataatcaacctctggatt
acaaaatttgtgaaagattgactggtattcttaactatgttgctccttttacgctatgtggata
cgctgctttaatgcctttgtatcatgctattgcttcccgtatggctttcattttctcctccttg
tataaatcctggttgctgtctctttatgaggagttgtggcccgttgtcaggcaacgtggcgtgg
tgtgcactgtgtttgctgacgcaacccccactggttggggcattgccaccacctgtcagctcct
ttccgggactttcgctttccccctccctattgccacggcggaactcatcgccgcctgccttgcc
cgctgctggacaggggctcggctgttgggcactgacaattccgtggtgttgtcggggaaatcat
cgtcctttccttggctgctcgcctatgttgccacctggattctgcgcgggacgtccttctgcta
cgtcccttcggccctcaatccagcggaccttccttcccgcggcctgctgccggctctgcggcct
cttccgcgtcttcgccttcgccctcagacgagtcggatctccctttgggccgcctccccgcatc
gataccgagcgctgctcgagagatctacgggtggcatccctgtgacccctccccagtgcctctc
ctggccctggaagttgccactccagtgcccaccagccttgtcctaataaaattaagttgcatca
ttttgtctgactaggtgtccttctataatattatggggtggaggggggtggtatggagcaaggg
gcaagttgggaagacaacctgtagggcctgcggggtctattgggaaccaagctggagtgcagtg
gcacaatcttggctcactgcaatctccgcctcctgggttcaagcgattctcctgcctcagcctc
ccgagttgttgggattccaggcatgcatgaccaggctcagctaatttttgtttttttggtagag
acggggtttcaccatattggccaggctggtctccaactcctaatctcaggtgatctacccacct
tggcctcccaaattgctgggattacaggcgtgaaccactgctcccttccctgtccttctgattt
tgtaggtaaccacgtgcggaccgagcggccgcaggaacccctagtgatggagttggccactccc
tctctgcgcgctcgctcgctcactgaggccgggcgaccaaaggtcgcccgacgcccgggctttg
cccgggcggcctcagtgagcgagcgagcgcgcagctgcctgcaggggcgcctgatgcggtattt
tctccttacgcatctgtgcggtatttcacaccgcatacgtcaaagcaaccatagtacgcgccct
gtagcggcgcattaagcgcggcgggtgtggtggttacgcgcagcgtgaccgctacacttgccag
cgccttagcgcccgctcctttcgctttcttcccttcctttctcgccacgttcgccggctttccc
cgtcaagctctaaatcgggggctccctttagggttccgatttagtgctttacggcacctcgacc
ccaaaaaacttgatttgggtgatggttcacgtagtgggccatcgccctgatagacggtttttcg
ccctttgacgttggagtccacgttctttaatagtggactcttgttccaaactggaacaacactc
aactctatctcgggctattcttttgatttataagggattttgccgatttcggtctattggttaa
aaaatgagctgatttaacaaaaatttaacgcgaattttaacaaaatattaacgtttacaatttt
atggtgcactctcagtacaatctgctctgatgccgcatagttaagccagccccgacacccgcca
acacccgctgacgcgccctgacgggcttgtctgctcccggcatccgcttacagacaagctgtga
ccgtctccgggagctgcatgtgtcagaggttttcaccgtcatcaccgaaacgcgcgagacgaaa
gggcctcgtgatacgcctatttttataggttaatgtcatgataataatggtttcttagacgtca
ggtggcacttttcggggaaatgtgcgcggaacccctatttgtttatttttctaaatacattcaa
atatgtatccgctcatgagacaataaccctgataaatgcttcaataatattgaaaaaggaagag
tatgagtattcaacatttccgtgtcgcccttattcccttttttgcggcattttgccttcctgtt
tttgctcacccagaaacgctggtgaaagtaaaagatgctgaagatcagttgggtgcacgagtgg
gttacatcgaactggatctcaacagcggtaagatccttgagagttttcgccccgaagaacgttt
tccaatgatgagcacttttaaagttctgctatgtggcgcggtattatcccgtattgacgccggg
caagagcaactcggtcgccgcatacactattctcagaatgacttggttgagtactcaccagtca
cagaaaagcatcttacggatggcatgacagtaagagaattatgcagtgctgccataaccatgag
tgataacactgcggccaacttacttctgacaacgatcggaggaccgaaggagctaaccgctttt
ttgcacaacatgggggatcatgtaactcgccttgatcgttgggaaccggagctgaatgaagcca
taccaaacgacgagcgtgacaccacgatgcctgtagcaatggcaacaacgttgcgcaaactatt
aactggcgaactacttactctagcttcccggcaacaattaatagactggatggaggcggataaa
gttgcaggaccacttctgcgctcggcccttccggctggctggtttattgctgataaatctggag
ccggtgagcgtgggtctcgcggtatcattgcagcactggggccagatggtaagccctcccgtat
cgtagttatctacacgacggggagtcaggcaactatggatgaacgaaatagacagatcgctgag
ataggtgcctcactgattaagcattggtaactgtcagaccaagtttactcatatatactttaga
ttgatttaaaacttcatttttaatttaaaaggatctaggtgaagatcctttttgataatctcat
gaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgc
taccagcggtggtttgtttgccggatcaagagctaccaactctttttccgaaggtaactggctt
cagcagagcgcagataccaaatactgttcttctagtgtagccgtagttaggccaccacttcaag
aactctgtagcaccgcctacatacctcgctctgctaatcctgttaccagtggctgctgccagtg
gcgataagtcgtgtcttaccgggttggactcaagacgatagttaccggataaggcgcagcggtc
gggctgaacggggggttcgtgcacacagcccagcttggagcgaacgacctacaccgaactgaga
tacctacagcgtgagctatgagaaagcgccacgcttcccgaagggagaaaggcggacaggtatc
cggtaagcggcagggtcggaacaggagagcgcacgagggagcttccagggggaaacgcctggta
tctttatagtcctgtcgggtttcgccacctctgacttgagcgtcgatttttgtgatgctcgtca
ggggggcggagcctatggaaaaacgccagcaacgcggcctttttacggttcctggccttttgct
ggccttttgctcacatgt

Plasmid #8 (see FIG. 26 for a map of the plasmid)

(SEQ ID NO: 101)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgtgtctagactgcagagggccctgcgtatgagtgcaagtgggttttag
gaccaggatgaggcggggTgggggtgcctacctgacgaccgaccccgacccactggacaagcac
ccaacccccattccccaaattgcgcatcccctatcagagagggggaggggaaacaggatgcggc
gaggcgcgtgcgcactgccagcttcagcaccgcggacagtgccttcgcccccgcctggcggcgc
gcgccaccgccgcctcagcactgaaggcgcgctgacgtcactcgccggtcccccgcaaactccc
cttcccggccaccttggtcgcgtccgcgccgccgccggcccagccggaccgcaccacgcgaggc
gcgagataggggggcacgggcgcgaccatctgcgctgcggcgccggcgactcagcgctgcctca
gtctgcggtgggcagcggaggagtcgtgtcgtgcctgagagcgcagtcgagaaggtaccGGATC
CGCCACCatggattacaaggatgacgacgataagGGCGGAGGTGGTTCTtccaaaaccatcgtt
ctttcggtcggcgaggctactcgcactctgactgagatccagtccaccgcagaccgtcagatct
tcgaagagaaggtcgggcctctggtgggtcggctgcgcctcacggcttcgctccgtcaaaacgg
agccaagaccgcgtatcgcgtcaacctaaaactggatcaggcggacgtcgttgattgctccacc
agcgtctgcggcgagcttccgaaagtgcgctacactcaggtatggtcgcacgacgtgacaatcg
ttgcgaatagcaccgaggcctcgcgcaaatcgttgtacgatttgaccaagtccctcgtcgcgac
ctcgcaggtcgaagatcttgtcgtcaaccttgtgccgctgggccgtGGCGGAGGTGGTTCTaag
ctgaaccctcctgatgagagtggccccggctgcatgagctgctgtgtgctctcctaagaattcg
atatcaagcttatcgataatcaacctctggattacaaaatttgtgaaagattgactggtattct
taactatgttgctccttttacgctatgtggatacgctgctttaatgcctttgtatcatgctatt
gcttcccgtatggctttcattttctcctccttgtataaatcctggttgctgtctctttatgagg
agttgtggcccgttgtcaggcaacgtggcgtggtgtgcactgtgtttgctgacgcaacccccac
tggttggggcattgccaccacctgtcagctcctttccgggactttcgctttccccctccctatt
gccacggcggaactcatcgccgcctgccttgcccgctgctggacaggggctcggctgttgggca
ctgacaattccgtggtgttgtcggggaaatcatcgtcctttccttggctgctcgcctatgttgc
cacctggattctgcgcgggacgtccttctgctacgtcccttcggccctcaatccagcggacctt
ccttcccgcggcctgctgccggctctgcggcctcttccgcgtcttcgccttcgccctcagacga
gtcggatctccctttgggccgcctccccgcatcgataccgagcgctgctcgagagatctacggg
tggcatccctgtgacccctccccagtgcctctcctggccctggaagttgccactccagtgccca
ccagccttgtcctaataaaattaagttgcatcattttgtctgactaggtgtccttctataatat
tatggggtggaggggggtggtatggagcaaggggcaagttgggaagacaacctgtagggcctgc
ggggtctattgggaaccaagctggagtgcagtggcacaatcttggctcactgcaatctccgcct
cctgggttcaagcgattctcctgcctcagcctcccgagttgttgggattccaggcatgcatgac
caggctcagctaatttttgtttttttggtagagacggggtttcaccatattggccaggctggtc
tccaactcctaatctcaggtgatctacccaccttggcctcccaaattgctgggattacaggcgt
gaaccactgctcccttccctgtccttctgattttgtaggtaaccacgtgcggaccgagcggccg
caggaacccctagtgatggagttggccactccctctctgcgcgctcgctcgctcactgaggccg
ggcgaccaaaggtcgcccgacgcccgggctttgcccgggcggcctcagtgagcgagcgagcgcg
cagctgcctgcaggggcgcctgatgcggtattttctccttacgcatctgtgcggtatttcacac
cgcatacgtcaaagcaaccatagtacgcgccctgtagcggcgcattaagcgcggcgggtgtggt
ggttacgcgcagcgtgaccgctacacttgccagcgccttagcgcccgctcctttcgctttcttc
ccttcctttctcgccacgttcgccggctttccccgtcaagctctaaatcgggggctccctttag
ggttccgatttagtgctttacggcacctcgaccccaaaaaacttgatttgggtgatggttcacg
tagtgggccatcgccctgatagacggtttttcgccctttgacgttggagtccacgttctttaat
agtggactcttgttccaaactggaacaacactcaactctatctcgggctattcttttgatttat
aagggattttgccgatttcggtctattggttaaaaaatgagctgatttaacaaaaatttaacgc
gaattttaacaaaatattaacgtttacaattttatggtgcactctcagtacaatctgctctgat
gccgcatagttaagccagccccgacacccgccaacacccgctgacgcgccctgacgggcttgtc
tgctcccggcatccgcttacagacaagctgtgaccgtctccgggagctgcatgtgtcagaggtt
ttcaccgtcatcaccgaaacgcgcgagacgaaagggcctcgtgatacgcctatttttataggtt
aatgtcatgataataatggtttcttagacgtcaggtggcacttttcggggaaatgtgcgcggaa
cccctatttgtttatttttctaaatacattcaaatatgtatccgctcatgagacaataaccctg
ataaatgcttcaataatattgaaaaaggaagagtatgagtattcaacatttccgtgtcgccctt
attcccttttttgcggcattttgccttcctgtttttgctcacccagaaacgctggtgaaagtaa
aagatgctgaagatcagttgggtgcacgagtgggttacatcgaactggatctcaacagcggtaa
gatccttgagagttttcgccccgaagaacgttttccaatgatgagcacttttaaagttctgcta
tgtggcgcggtattatcccgtattgacgccgggcaagagcaactcggtcgccgcatacactatt
ctcagaatgacttggttgagtactcaccagtcacagaaaagcatcttacggatggcatgacagt
aagagaattatgcagtgctgccataaccatgagtgataacactgcggccaacttacttctgaca
acgatcggaggaccgaaggagctaaccgcttttttgcacaacatgggggatcatgtaactcgcc
ttgatcgttgggaaccggagctgaatgaagccataccaaacgacgagcgtgacaccacgatgcc
tgtagcaatggcaacaacgttgcgcaaactattaactggcgaactacttactctagcttcccgg
caacaattaatagactggatggaggcggataaagttgcaggaccacttctgcgctcggcccttc
cggctggctggtttattgctgataaatctggagccggtgagcgtgggtctcgcggtatcattgc
agcactggggccagatggtaagccctcccgtatcgtagttatctacacgacggggagtcaggca
actatggatgaacgaaatagacagatcgctgagataggtgcctcactgattaagcattggtaac
tgtcagaccaagtttactcatatatactttagattgatttaaaacttcatttttaatttaaaag
gatctaggtgaagatcctttttgataatctcatgaccaaaatcccttaacgtgagttttcgttc
cactgagcgtcagaccccgtagaaaagatcaaaggatcttcttgagatcctttttttctgcgcg
taatctgctgcttgcaaacaaaaaaaccaccgctaccagcggtggtttgtttgccggatcaaga
gctaccaactctttttccgaaggtaactggcttcagcagagcgcagataccaaatactgttctt
ctagtgtagccgtagttaggccaccacttcaagaactctgtagcaccgcctacatacctcgctc
tgctaatcctgttaccagtggctgctgccagtggcgataagtcgtgtcttaccgggttggactc
aagacgatagttaccggataaggcgcagcggtcgggctgaacggggggttcgtgcacacagccc
agcttggagcgaacgacctacaccgaactgagatacctacagcgtgagctatgagaaagcgcca
cgcttcccgaagggagaaaggcggacaggtatccggtaagcggcagggtcggaacaggagagcg
cacgagggagcttccagggggaaacgcctggtatctttatagtcctgtcgggtttcgccacctc
tgacttgagcgtcgatttttgtgatgctcgtcaggggggcggagcctatggaaaaacgccagca
acgcggcctttttacggttcctggccttttgctggccttttgctcacatgt

Plasmid #9 (see FIG. 27 for a map of the plasmid)

(SEQ ID NO: 102)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccggggccctgaagaagggccccggtccgatactctgatgatgggtcccCCTAGG
TTAAGGATGCACCGACGGGACGTTCTATGAGGACGAATCTCCCGCTTATAAGATTCTATAAACT
GTGCGGTCCTTCAATTGgggtcccatcattcatggcaagtggccgcggtcggcgtggactgtag
aacactgccaatgccggtcccaagcccggataaaaGTGGAGGGTACAGTCCACGCtttttttct
agactgcagagggccctgcgtatgagtgcaagtgggttttaggaccaggatgaggcggggtggg
ggtgcctacctgacgaccgaccccgacccactggacaagcacccaacccccattccccaaattg
cgcatcccctatcagagagggggaggggaaacaggatgcggcgaggcgcgtgcgcactgccagc
ttcagcaccgcggacagtgccttcgcccccgcctggcggcgcgcgccaccgccgcctcagcact
gaaggcgcgctgacgtcactcgccggtcccccgcaaactccccttcccggccaccttggtcgcg
tccgcgccgccgccggcccagccggaccgcaccacgcgaggcgcgagataggggggcacgggcg
cgaccatctgcgctgcggcgccggcgactcagcgctgcctcagtctgcggtgggcagcggagga
gtcgtgtcgtgcctgagagcgcagtcgagaaggtaccGCCACCatggattacaaggatgacgac
gataagGGCGGAGGTGGTTCTgacgcacaaacacgacgacgtgagcgtcgcgctgagaaacaag
ctcaatggaaagctgcaaacGGCGGAGGTGGTTCTaagctgaaccctcctgatgagagtggccc
cggctgcatgagctgctgtgtgctctcctaagaattcgatatcaagcttatcgataatcaacct
ctggattacaaaatttgtgaaagattgactggtattcttaactatgttgctccttttacgctat
gtggatacgctgctttaatgcctttgtatcatgctattgcttcccgtatggctttcattttctc
ctccttgtataaatcctggttgctgtctctttatgaggagttgtggcccgttgtcaggcaacgt
ggcgtggtgtgcactgtgtttgctgacgcaacccccactggttggggcattgccaccacctgtc
agctcctttccgggactttcgctttccccctccctattgccacggcggaactcatcgccgcctg
ccttgcccgctgctggacaggggctcggctgttgggcactgacaattccgtggtgttgtcgggg
aaatcatcgtcctttccttggctgctcgcctatgttgccacctggattctgcgcgggacgtcct
tctgctacgtcccttcggccctcaatccagcggaccttccttcccgcggcctgctgccggctct
gcggcctcttccgcgtcttcgccttcgccctcagacgagtcggatctccctttgggccgcctcc
ccgcatcgataccgagcgctgctcgagagatctacgggtggcatccctgtgacccctccccagt
gcctctcctggccctggaagttgccactccagtgcccaccagccttgtcctaataaaattaagt
tgcatcattttgtctgactaggtgtccttctataatattatggggtggaggggggtggtatgga
gcaaggggcaagttgggaagacaacctgtagggcctgcggggtctattgggaaccaagctggag
tgcagtggcacaatcttggctcactgcaatctccgcctcctgggttcaagcgattctcctgcct
cagcctcccgagttgttgggattccaggcatgcatgaccaggctcagctaatttttgttttttt
ggtagagacggggtttcaccatattggccaggctggtctccaactcctaatctcaggtgatcta
cccaccttggcctcccaaattgctgggattacaggcgtgaaccactgctcccttccctgtcctt
ctgattttgtaggtaaccacgtgcggaccgagcggccgcaggaacccctagtgatggagttggc
cactccctctctgcgcgctcgctcgctcactgaggccgggcgaccaaaggtcgcccgacgcccg
ggctttgcccgggcggcctcagtgagcgagcgagcgcgcagctgcctgcaggggcgcctgatgc
ggtattttctccttacgcatctgtgcggtatttcacaccgcatacgtcaaagcaaccatagtac
gcgccctgtagcggcgcattaagcgcggcgggtgtggtggttacgcgcagcgtgaccgctacac
ttgccagcgccttagcgcccgctcctttcgctttcttcccttcctttctcgccacgttcgccgg
ctttccccgtcaagctctaaatcgggggctccctttagggttccgatttagtgctttacggcac
ctcgaccccaaaaaacttgatttgggtgatggttcacgtagtgggccatcgccctgatagacgg
tttttcgccctttgacgttggagtccacgttctttaatagtggactcttgttccaaactggaac
aacactcaactctatctcgggctattcttttgatttataagggattttgccgatttcggtctat
tggttaaaaaatgagctgatttaacaaaaatttaacgcgaattttaacaaaatattaacgttta
caattttatggtgcactctcagtacaatctgctctgatgccgcatagttaagccagccccgaca
cccgccaacacccgctgacgcgccctgacgggcttgtctgctcccggcatccgcttacagacaa
gctgtgaccgtctccgggagctgcatgtgtcagaggttttcaccgtcatcaccgaaacgcgcga
gacgaaagggcctcgtgatacgcctatttttataggttaatgtcatgataataatggtttctta
gacgtcaggtggcacttttcggggaaatgtgcgcggaacccctatttgtttatttttctaaata
cattcaaatatgtatccgctcatgagacaataaccctgataaatgcttcaataatattgaaaaa
ggaagagtatgagtattcaacatttccgtgtcgcccttattcccttttttgcggcattttgcct
tcctgtttttgctcacccagaaacgctggtgaaagtaaaagatgctgaagatcagttgggtgca
cgagtgggttacatcgaactggatctcaacagcggtaagatccttgagagttttcgccccgaag
aacgttttccaatgatgagcacttttaaagttctgctatgtggcgcggtattatcccgtattga
cgccgggcaagagcaactcggtcgccgcatacactattctcagaatgacttggttgagtactca
ccagtcacagaaaagcatcttacggatggcatgacagtaagagaattatgcagtgctgccataa
ccatgagtgataacactgcggccaacttacttctgacaacgatcggaggaccgaaggagctaac
cgcttttttgcacaacatgggggatcatgtaactcgccttgatcgttgggaaccggagctgaat
gaagccataccaaacgacgagcgtgacaccacgatgcctgtagcaatggcaacaacgttgcgca
aactattaactggcgaactacttactctagcttcccggcaacaattaatagactggatggaggc
ggataaagttgcaggaccacttctgcgctcggcccttccggctggctggtttattgctgataaa
tctggagccggtgagcgtgggtctcgcggtatcattgcagcactggggccagatggtaagccct
cccgtatcgtagttatctacacgacggggagtcaggcaactatggatgaacgaaatagacagat
cgctgagataggtgcctcactgattaagcattggtaactgtcagaccaagtttactcatatata
ctttagattgatttaaaacttcatttttaatttaaaaggatctaggtgaagatcctttttgata
atctcatgaccaaaatcccttaacgtgagttttcgttccactgagcgtcagaccccgtagaaaa
gatcaaaggatcttcttgagatcctttttttctgcgcgtaatctgctgcttgcaaacaaaaaaa
ccaccgctaccagcggtggtttgtttgccggatcaagagctaccaactctttttccgaaggtaa
ctggcttcagcagagcgcagataccaaatactgttcttctagtgtagccgtagttaggccacca
cttcaagaactctgtagcaccgcctacatacctcgctctgctaatcctgttaccagtggctgct
gccagtggcgataagtcgtgtcttaccgggttggactcaagacgatagttaccggataaggcgc
agcggtcgggctgaacggggggttcgtgcacacagcccagcttggagcgaacgacctacaccga
actgagatacctacagcgtgagctatgagaaagcgccacgcttcccgaagggagaaaggcggac
aggtatccggtaagcggcagggtcggaacaggagagcgcacgagggagcttccagggggaaacg
cctggtatctttatagtcctgtcgggtttcgccacctctgacttgagcgtcgatttttgtgatg
ctcgtcaggggggcggagcctatggaaaaacgccagcaacgcggcctttttacggttcctggcc
ttttgctggccttttgctcacatgt

Plasmid #10 (see FIG. 28 for a map of the plasmid)

(SEQ ID NO: 103)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgacatgaggatcacccatgtcggtccgatactctgatgatgggtcccCCTAGG
TTAAGGATGCACCGACGGGACGTTCTATGAGGACGAATCTCCCGCTTATAAGATTCTATAAACT
GTGCGGTCCTTCAATTGgggtcccatcattcatggcaagtggccgcggtcggcgtggactgtag
aacactgccaatgccggtcccaagcccggataaaaGTGGAGGGTACAGTCCACGCtttttttct
agactgcagagggccctgcgtatgagtgcaagtgggttttaggaccaggatgaggcggggtggg
ggtgcctacctgacgaccgaccccgacccactggacaagcacccaacccccattccccaaattg
cgcatcccctatcagagagggggaggggaaacaggatgcggcgaggcgcgtgcgcactgccagc
ttcagcaccgcggacagtgccttcgcccccgcctggcggcgcgcgccaccgccgcctcagcact
gaaggcgcgctgacgtcactcgccggtcccccgcaaactccccttcccggccaccttggtcgcg
tccgcgccgccgccggcccagccggaccgcaccacgcgaggcgcgagataggggggcacgggcg
cgaccatctgcgctgcggcgccggcgactcagcgctgcctcagtctgcggtgggcagcggagga
gtcgtgtcgtgcctgagagcgcagtcgagaaggtaccGGATCCGCCACCatggattacaaggat
gacgacgataagGGCGGAGGTGGTTCTgcttctaactttactcagttcgttctcgtcgacaatg
gcggaactggcgacgtgactgtcgccccaagcaacttcgctaacggggtcgctgaatggatcag
ctctaactcgcgttcacaggcttacaaagtaacctgtagcgttcgtcagagctctgcgcagaat
cgcaaatacaccatcaaagtcgaggtgcctaaaggcgcatggcgttcgtacttaaatatggaac
taaccattccaattttctccacgaactccgactgcgagcttattgttaaggcaatgcaaggtct
cctaaaagatggaaacccgattccctcagcaatcgcagcaaactccggcatctacGGCGGAGGT
GGTTCTaagctgaaccctcctgatgagagtggccccggctgcatgagctgctgtgtgctctcct
aagaattcgatatcaagcttatcgataatcaacctctggattacaaaatttgtgaaagattgac
tggtattcttaactatgttgctccttttacgctatgtggatacgctgctttaatgcctttgtat
catgctattgcttcccgtatggctttcattttctcctccttgtataaatcctggttgctgtctc
tttatgaggagttgtggcccgttgtcaggcaacgtggcgtggtgtgcactgtgtttgctgacgc
aacccccactggttggggcattgccaccacctgtcagctcctttccgggactttcgctttcccc
ctccctattgccacggcggaactcatcgccgcctgccttgcccgctgctggacaggggctcggc
tgttgggcactgacaattccgtggtgttgtcggggaaatcatcgtcctttccttggctgctcgc
ctatgttgccacctggattctgcgcgggacgtccttctgctacgtcccttcggccctcaatcca
gcggaccttccttcccgcggcctgctgccggctctgcggcctcttccgcgtcttcgccttcgcc
ctcagacgagtcggatctccctttgggccgcctccccgcatcgataccgagcgctgctcgagag
atctacgggtggcatccctgtgacccctccccagtgcctctcctggccctggaagttgccactc
cagtgcccaccagccttgtcctaataaaattaagttgcatcattttgtctgactaggtgtcctt
ctataatattatggggtggaggggggTggtatggagcaaggggcaagttgggaagacaacctgt
agggcctgcggggtctattgggaaccaagctggagtgcagtggcacaatcttggctcactgcaa
tctccgcctcctgggttcaagcgattctcctgcctcagcctcccgagttgttgggattccaggc
atgcatgaccaggctcagctaatttttgtttttttggtagagacggggtttcaccatattggcc
aggctggtctccaactcctaatctcaggtgatctacccaccttggcctcccaaattgctgggat
tacaggcgtgaaccactgctcccttccctgtccttctgattttgtaggtaaccacgtgcggacc
gagcggccgcaggaacccctagtgatggagttggccactccctctctgcgcgctcgctcgctca
ctgaggccgggcgaccaaaggtcgcccgacgcccgggctttgcccgggcggcctcagtgagcga
gcgagcgcgcagctgcctgcaggggcgcctgatgcggtattttctccttacgcatctgtgcggt
atttcacaccgcatacgtcaaagcaaccatagtacgcgccctgtagcggcgcattaagcgcggc
gggtgtggtggttacgcgcagcgtgaccgctacacttgccagcgccttagcgcccgctcctttc
gctttcttcccttcctttctcgccacgttcgccggctttccccgtcaagctctaaatcgggggc
tccctttagggttccgatttagtgctttacggcacctcgaccccaaaaaacttgatttgggtga
tggttcacgtagtgggccatcgccctgatagacggtttttcgccctttgacgttggagtccacg
ttctttaatagtggactcttgttccaaactggaacaacactcaactctatctcgggctattctt
ttgatttataagggattttgccgatttcggtctattggttaaaaaatgagctgatttaacaaaa
atttaacgcgaattttaacaaaatattaacgtttacaattttatggtgcactctcagtacaatc
tgctctgatgccgcatagttaagccagccccgacacccgccaacacccgctgacgcgccctgac
gggcttgtctgctcccggcatccgcttacagacaagctgtgaccgtctccgggagctgcatgtg
tcagaggttttcaccgtcatcaccgaaacgcgcgagacgaaagggcctcgtgatacgcctattt
ttataggttaatgtcatgataataatggtttcttagacgtcaggtggcacttttcggggaaatg
tgcgcggaacccctatttgtttatttttctaaatacattcaaatatgtatccgctcatgagaca
ataaccctgataaatgcttcaataatattgaaaaaggaagagtatgagtattcaacatttccgt
gtcgcccttattcccttttttgcggcattttgccttcctgtttttgctcacccagaaacgctgg
tgaaagtaaaagatgctgaagatcagttgggtgcacgagtgggttacatcgaactggatctcaa
cagcggtaagatccttgagagttttcgccccgaagaacgttttccaatgatgagcacttttaaa
gttctgctatgtggcgcggtattatcccgtattgacgccgggcaagagcaactcggtcgccgca
tacactattctcagaatgacttggttgagtactcaccagtcacagaaaagcatcttacggatgg
catgacagtaagagaattatgcagtgctgccataaccatgagtgataacactgcggccaactta
cttctgacaacgatcggaggaccgaaggagctaaccgcttttttgcacaacatgggggatcatg
taactcgccttgatcgttgggaaccggagctgaatgaagccataccaaacgacgagcgtgacac
cacgatgcctgtagcaatggcaacaacgttgcgcaaactattaactggcgaactacttactcta
gcttcccggcaacaattaatagactggatggaggcggataaagttgcaggaccacttctgcgct
cggcccttccggctggctggtttattgctgataaatctggagccggtgagcgtgggtctcgcgg
tatcattgcagcactggggccagatggtaagccctcccgtatcgtagttatctacacgacgggg
agtcaggcaactatggatgaacgaaatagacagatcgctgagataggtgcctcactgattaagc
attggtaactgtcagaccaagtttactcatatatactttagattgatttaaaacttcattttta
atttaaaaggatctaggtgaagatcctttttgataatctcatgaccaaaatcccttaacgtgag
ttttcgttccactgagcgtcagaccccgtagaaaagatcaaaggatcttcttgagatccttttt
ttctgcgcgtaatctgctgcttgcaaacaaaaaaaccaccgctaccagcggtggtttgtttgcc
ggatcaagagctaccaactctttttccgaaggtaactggcttcagcagagcgcagataccaaat
actgttcttctagtgtagccgtagttaggccaccacttcaagaactctgtagcaccgcctacat
acctcgctctgctaatcctgttaccagtggctgctgccagtggcgataagtcgtgtcttaccgg
gttggactcaagacgatagttaccggataaggcgcagcggtcgggctgaacggggggttcgtgc
acacagcccagcttggagcgaacgacctacaccgaactgagatacctacagcgtgagctatgag
aaagcgccacgcttcccgaagggagaaaggcggacaggtatccggtaagcggcagggtcggaac
aggagagcgcacgagggagcttccagggggaaacgcctggtatctttatagtcctgtcgggttt
cgccacctctgacttgagcgtcgatttttgtgatgctcgtcaggggggcggagcctatggaaaa
acgccagcaacgcggcctttttacggttcctggccttttgctggccttttgctcacatgt

Plasmid #11 (see FIG. 29 for a map of the plasmid)

(SEQ ID NO: 104)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggccctgcgtatgagtgcaagtgggttttaggaccaggatgaggcgg
ggtgggggtgcctacctgacgaccgaccccgacccactggacaagcacccaacccccattcccc
aaattgcgcatcccctatcagagagggggaggggaaacaggatgcggcgaggcgcgtgcgcact
gccagcttcagcaccgcggacagtgccttcgcccccgcctggcggcgcgcgccaccgccgcctc
agcactgaaggcgcgctgacgtcactcgccggtcccccgcaaactccccttcccggccaccttg
gtcgcgtccgcgccgccgccggcccagccggaccgcaccacgcgaggcgcgagataggggggca
cgggcgcgaccatctgcgctgcggcgccggcgactcagcgctgcctcagtctgcggtgggcagc
ggaggagtcgtgtcgtgcctgagagcgcagtcgagaaggtaccGGATCCGCCACCatggattac
aaggatgacgacgataagGGCGGAGGTGGTTCTtccaaaaccatcgttctttcggtcggcgagg
ctactcgcactctgactgagatccagtccaccgcagaccgtcagatcttcgaagagaaggtcgg
gcctctggtgggtcggctgcgcctcacggcttcgctccgtcaaaacggagccaagaccgcgtat
cgcgtcaacctaaaactggatcaggcggacgtcgttgattgctccaccagcgtctgcggcgagc
ttccgaaagtgcgctacactcaggtatggtcgcacgacgtgacaatcgttgcgaatagcaccga
ggcctcgcgcaaatcgttgtacgatttgaccaagtccctcgtcgcgacctcgcaggtcgaagat
cttgtcgtcaaccttgtgccgctgggccgtGGCGGAGGTGGTTCTaagctgaaccctcctgatg
agagtggccccggctgcatgagctgctgtgtgctctcctaagaattcgatatcaagcttatcga
taatcaacctctggattacaaaatttgtgaaagattgactggtattcttaactatgttgctcct
tttacgctatgtggatacgctgctttaatgcctttgtatcatgctattgcttcccgtatggctt
tcattttctcctccttgtataaatcctggttgctgtctctttatgaggagttgtggcccgttgt
caggcaacgtggcgtggtgtgcactgtgtttgctgacgcaacccccactggttggggcattgcc
accacctgtcagctcctttccgggactttcgctttccccctccctattgccacggcggaactca
tcgccgcctgccttgcccgctgctggacaggggctcggctgttgggcactgacaattccgtggt
gttgtcggggaaatcatcgtcctttccttggctgctcgcctatgttgccacctggattctgcgc
gggacgtccttctgctacgtcccttcggccctcaatccagcggaccttccttcccgcggcctgc
tgccggctctgcggcctcttccgcgtcttcgccttcgccctcagacgagtcggatctccctttg
ggccgcctccccgcatcgataccgagcgctgctcgagagatctacgggtggcatccctgtgacc
cctccccagtgcctctcctggccctggaagttgccactccagtgcccaccagccttgtcctaat
aaaattaagttgcatcattttgtctgactaggtgtccttctataatattatggggTggaggggg
gtggtatggagcaaggggcaagttgggaagacaacctgtagggcctgcggggtctattgggaac
caagctggagtgcagtggcacaatcttggctcactgcaatctccgcctcctgggttcaagcgat
tctcctgcctcagcctcccgagttgttgggattccaggcatgcatgaccaggctcagctaattt
ttgtttttttggtagagacggggtttcaccatattggccaggctggtctccaactcctaatctc
aggtgatctacccaccttggcctcccaaattgctgggattacaggcgtgaaccactgctccctt
ccctgtccttctgattttgtaggtaaccacgtgcggaccgagcggccgcaggaacccctagtga
tggagttggccactccctctctgcgcgctcgctcgctcactgaggccgggcgaccaaaggtcgc
ccgacgcccgggctttgcccgggcggcctcagtgagcgagcgagcgcgcagctgcctgcagggg
cgcctgatgcggtattttctccttacgcatctgtgcggtatttcacaccgcatacgtcaaagca
accatagtacgcgccctgtagcggcgcattaagcgcggcgggtgtggtggttacgcgcagcgtg
accgctacacttgccagcgccttagcgcccgctcctttcgctttcttcccttcctttctcgcca
cgttcgccggctttccccgtcaagctctaaatcgggggctccctttagggttccgatttagtgc
tttacggcacctcgaccccaaaaaacttgatttgggtgatggttcacgtagtgggccatcgccc
tgatagacggtttttcgccctttgacgttggagtccacgttctttaatagtggactcttgttcc
aaactggaacaacactcaactctatctcgggctattcttttgatttataagggattttgccgat
ttcggtctattggttaaaaaatgagctgatttaacaaaaatttaacgcgaattttaacaaaata
ttaacgtttacaattttatggtgcactctcagtacaatctgctctgatgccgcatagttaagcc
agccccgacacccgccaacacccgctgacgcgccctgacgggcttgtctgctcccggcatccgc
ttacagacaagctgtgaccgtctccgggagctgcatgtgtcagaggttttcaccgtcatcaccg
aaacgcgcgagacgaaagggcctcgtgatacgcctatttttataggttaatgtcatgataataa
tggtttcttagacgtcaggtggcacttttcggggaaatgtgcgcggaacccctatttgtttatt
tttctaaatacattcaaatatgtatccgctcatgagacaataaccctgataaatgcttcaataa
tattgaaaaaggaagagtatgagtattcaacatttccgtgtcgcccttattcccttttttgcgg
cattttgccttcctgtttttgctcacccagaaacgctggtgaaagtaaaagatgctgaagatca
gttgggtgcacgagtgggttacatcgaactggatctcaacagcggtaagatccttgagagtttt
cgccccgaagaacgttttccaatgatgagcacttttaaagttctgctatgtggcgcggtattat
cccgtattgacgccgggcaagagcaactcggtcgccgcatacactattctcagaatgacttggt
tgagtactcaccagtcacagaaaagcatcttacggatggcatgacagtaagagaattatgcagt
gctgccataaccatgagtgataacactgcggccaacttacttctgacaacgatcggaggaccga
aggagctaaccgcttttttgcacaacatgggggatcatgtaactcgccttgatcgttgggaacc
ggagctgaatgaagccataccaaacgacgagcgtgacaccacgatgcctgtagcaatggcaaca
acgttgcgcaaactattaactggcgaactacttactctagcttcccggcaacaattaatagact
ggatggaggcggataaagttgcaggaccacttctgcgctcggcccttccggctggctggtttat
tgctgataaatctggagccggtgagcgtgggtctcgcggtatcattgcagcactggggccagat
ggtaagccctcccgtatcgtagttatctacacgacggggagtcaggcaactatggatgaacgaa
atagacagatcgctgagataggtgcctcactgattaagcattggtaactgtcagaccaagttta
ctcatatatactttagattgatttaaaacttcatttttaatttaaaaggatctaggtgaagatc
ctttttgataatctcatgaccaaaatcccttaacgtgagttttcgttccactgagcgtcagacc
ccgtagaaaagatcaaaggatcttcttgagatcctttttttctgcgcgtaatctgctgcttgca
aacaaaaaaaccaccgctaccagcggtggtttgtttgccggatcaagagctaccaactcttttt
ccgaaggtaactggcttcagcagagcgcagataccaaatactgttcttctagtgtagccgtagt
taggccaccacttcaagaactctgtagcaccgcctacatacctcgctctgctaatcctgttacc
agtggctgctgccagtggcgataagtcgtgtcttaccgggttggactcaagacgatagttaccg
gataaggcgcagcggtcgggctgaacggggggttcgtgcacacagcccagcttggagcgaacga
cctacaccgaactgagatacctacagcgtgagctatgagaaagcgccacgcttcccgaagggag
aaaggcggacaggtatccggtaagcggcagggtcggaacaggagagcgcacgagggagcttcca
gggggaaacgcctggtatctttatagtcctgtcgggtttcgccacctctgacttgagcgtcgat
ttttgtgatgctcgtcaggggggcggagcctatggaaaaacgccagcaacgcggcctttttacg
gttcctggccttttgctggccttttgctcacatgt

Plasmid #12 (see FIG. 30 for a map of the plasmid)

(SEQ ID NO: 105)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccGGGCACTCTTCCGTGGTCTGGTGGATAAATTCGttgccatgtgtatcggtccgGG
AGCAGACGATATGGCGTCGCTCCcggtccgatactctgatgatgggtcccCCTAGGTTAAGGAT
GCACCGACGGGACGTTCTATGAGGACGAATCTCCCGCTTATAAGATTCTATAAACTGTGCGGTC
CTTCAATTGgggtcccatcattcatggcaaCGACGTCAGACCACGGGGGAGTGCCCtttttttt
ctagactgcagagggccctgcgtatgagtgcaagtgggttttaggaccaggatgaggcggggtg
ggggtgcctacctgacgaccgaccccgacccactggacaagcacccaacccccattccccaaat
tgcgcatcccctatcagagagggggaggggaaacaggatgcggcgaggcgcgtgcgcactgcca
gcttcagcaccgcggacagtgccttcgcccccgcctggcggcgcgcgccaccgccgcctcagca
ctgaaggcgcgctgacgtcactcgccggtcccccgcaaactccccttcccggccaccttggtcg
cgtccgcgccgccgccggcccagccggaccgcaccacgcgaggcgcgagataggggggcacggg
cgcgaccatctgcgctgcggcgccggcgactcagcgctgcctcagtctgcggtgggcagcggag
gagtcgtgtcgtgcctgagagcgcagtcgagaaggtaccGGATCCGCCACCatggattacaagg
atgacgacgataagGGCGGAGGTGGTTCTtccaaaaccatcgttctttcggtcggcgaggctac
tcgcactctgactgagatccagtccaccgcagaccgtcagatcttcgaagagaaggtcgggcct
ctggtgggtcggctgcgcctcacggcttcgctccgtcaaaacggagccaagaccgcgtatcgcg
tcaacctaaaactggatcaggcggacgtcgttgattgctccaccagcgtctgcggcgagcttcc
gaaagtgcgctacactcaggtatggtcgcacgacgtgacaatcgttgcgaatagcaccgaggcc
tcgcgcaaatcgttgtacgatttgaccaagtccctcgtcgcgacctcgcaggtcgaagatcttg
tcgtcaaccttgtgccgctgggccgtGGCGGAGGTGGTTCTaagctgaaccctcctgatgagag
tggccccggctgcatgagctgctgtgtgctctcctaagaattcgatatcaagcttatcgataat
caacctctggattacaaaatttgtgaaagattgactggtattcttaactatgttgctcctttta
cgctatgtggatacgctgctttaatgcctttgtatcatgctattgcttcccgtatggctttcat
tttctcctccttgtataaatcctggttgctgtctctttatgaggagttgtggcccgttgtcagg
caacgtggcgtggtgtgcactgtgtttgctgacgcaacccccactggttggggcattgccacca
cctgtcagctcctttccgggactttcgctttccccctccctattgccacggcggaactcatcgc
cgcctgccttgcccgctgctggacaggggctcggctgttgggcactgacaattccgtggtgttg
tcggggaaatcatcgtcctttccttggctgctcgcctatgttgccacctggattctgcgcggga
cgtccttctgctacgtcccttcggccctcaatccagcggaccttccttcccgcggcctgctgcc
ggctctgcggcctcttccgcgtcttcgccttcgccctcagacgagtcggatctccctttgggcc
gcctccccgcatcgataccgagcgctgctcgagagatctacgggtggcatccctgtgacccctc
cccagtgcctctcctggccctggaagttgccactccagtgcccaccagccttgtcctaataaaa
ttaagttgcatcattttgtctgactaggtgtccttctataatattatggggtggaggggggtgg
tatggagcaaggggcaagttgggaagacaacctgtagggcctgcggggtctattgggaaccaag
ctggagtgcagtggcacaatcttggctcactgcaatctccgcctcctgggttcaagcgattctc
ctgcctcagcctcccgagttgttgggattccaggcatgcatgaccaggctcagctaatttttgt
ttttttggtagagacggggtttcaccatattggccaggctggtctccaactcctaatctcaggt
gatctacccaccttggcctcccaaattgctgggattacaggcgtgaaccactgctcccttccct
gtccttctgattttgtaggtaaccacgtgcggaccgagcggccgcaggaacccctagtgatgga
gttggccactccctctctgcgcgctcgctcgctcactgaggccgggcgaccaaaggtcgcccga
cgcccgggctttgcccgggcggcctcagtgagcgagcgagcgcgcagctgcctgcaggggcgcc
tgatgcggtattttctccttacgcatctgtgcggtatttcacaccgcatacgtcaaagcaacca
tagtacgcgccctgtagcggcgcattaagcgcggcgggtgtggtggttacgcgcagcgtgaccg
ctacacttgccagcgccttagcgcccgctcctttcgctttcttcccttcctttctcgccacgtt
cgccggctttccccgtcaagctctaaatcgggggctccctttagggttccgatttagtgcttta
cggcacctcgaccccaaaaaacttgatttgggtgatggttcacgtagtgggccatcgccctgat
agacggtttttcgccctttgacgttggagtccacgttctttaatagtggactcttgttccaaac
tggaacaacactcaactctatctcgggctattcttttgatttataagggattttgccgatttcg
gtctattggttaaaaaatgagctgatttaacaaaaatttaacgcgaattttaacaaaatattaa
cgtttacaattttatggtgcactctcagtacaatctgctctgatgccgcatagttaagccagcc
ccgacacccgccaacacccgctgacgcgccctgacgggcttgtctgctcccggcatccgcttac
agacaagctgtgaccgtctccgggagctgcatgtgtcagaggttttcaccgtcatcaccgaaac
gcgcgagacgaaagggcctcgtgatacgcctatttttataggttaatgtcatgataataatggt
ttcttagacgtcaggtggcacttttcggggaaatgtgcgcggaacccctatttgtttatttttc
taaatacattcaaatatgtatccgctcatgagacaataaccctgataaatgcttcaataatatt
gaaaaaggaagagtatgagtattcaacatttccgtgtcgcccttattcccttttttgcggcatt
ttgccttcctgtttttgctcacccagaaacgctggtgaaagtaaaagatgctgaagatcagttg
ggtgcacgagtgggttacatcgaactggatctcaacagcggtaagatccttgagagttttcgcc
ccgaagaacgttttccaatgatgagcacttttaaagttctgctatgtggcgcggtattatcccg
tattgacgccgggcaagagcaactcggtcgccgcatacactattctcagaatgacttggttgag
tactcaccagtcacagaaaagcatcttacggatggcatgacagtaagagaattatgcagtgctg
ccataaccatgagtgataacactgcggccaacttacttctgacaacgatcggaggaccgaagga
gctaaccgcttttttgcacaacatgggggatcatgtaactcgccttgatcgttgggaaccggag
ctgaatgaagccataccaaacgacgagcgtgacaccacgatgcctgtagcaatggcaacaacgt
tgcgcaaactattaactggcgaactacttactctagcttcccggcaacaattaatagactggat
ggaggcggataaagttgcaggaccacttctgcgctcggcccttccggctggctggtttattgct
gataaatctggagccggtgagcgtgggtctcgcggtatcattgcagcactggggccagatggta
agccctcccgtatcgtagttatctacacgacggggagtcaggcaactatggatgaacgaaatag
acagatcgctgagataggtgcctcactgattaagcattggtaactgtcagaccaagtttactca
tatatactttagattgatttaaaacttcatttttaatttaaaaggatctaggtgaagatccttt
ttgataatctcatgaccaaaatcccttaacgtgagttttcgttccactgagcgtcagaccccgt
agaaaagatcaaaggatcttcttgagatcctttttttctgcgcgtaatctgctgcttgcaaaca
aaaaaaccaccgctaccagcggtggtttgtttgccggatcaagagctaccaactctttttccga
aggtaactggcttcagcagagcgcagataccaaatactgttcttctagtgtagccgtagttagg
ccaccacttcaagaactctgtagcaccgcctacatacctcgctctgctaatcctgttaccagtg
gctgctgccagtggcgataagtcgtgtcttaccgggttggactcaagacgatagttaccggata
aggcgcagcggtcgggctgaacggggggttcgtgcacacagcccagcttggagcgaacgaccta
caccgaactgagatacctacagcgtgagctatgagaaagcgccacgcttcccgaagggagaaag
gcggacaggtatccggtaagcggcagggtcggaacaggagagcgcacgagggagcttccagggg
gaaacgcctggtatctttatagtcctgtcgggtttcgccacctctgacttgagcgtcgattttt
gtgatgctcgtcaggggggcggagcctatggaaaaacgccagcaacgcggcctttttacggttc
ctggccttttgctggccttttgctcacatgt

Plasmid #13 (see FIG. 31 for a map of the plasmid)

(SEQ ID NO: 106)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatCCTAGGT
TAAGGATGCACCGACGGGACGTTCTATGAGGACGAATCTCCCGCTTATAAGATTCTATAAACTG
TGCGGTCCTTCAATTGgggtcccCACTAACCTAAGACAGGAGGGCCGGGAAACCTGCCTAATCC
AATGACGGGTAATAGTGgggacccatcattcatggcaagtggccgcggtcggcgtggactgtag
aacactgccaatgccggtcccaagcccggataaaaGTGGAGGGTACAGTCCACGCtttttttct
agactgcagagggccctgcgtatgagtgcaagtgggttttaggaccaggatgaggcggggtggg
ggtgcctacctgacgaccgaccccgacccactggacaagcacccaacccccattccccaaattg
cgcatcccctatcagagagggggaggggaaacaggatgcggcgaggcgcgtgcgcactgccagc
ttcagcaccgcggacagtgccttcgcccccgcctggcggcgcgcgccaccgccgcctcagcact
gaaggcgcgctgacgtcactcgccggtcccccgcaaactccccttcccggccaccttggtcgcg
tccgcgccgccgccggcccagccggaccgcaccacgcgaggcgcgagataggggggcacgggcg
cgaccatctgcgctgcggcgccggcgactcagcgctgcctcagtctgcggtgggcagcggagga
gtcgtgtcgtgcctgagagcgcagtcgagaaggtaccGGATCCGCCACCatggattacaaggat
gacgacgataagGGCGGAGGTGGTTCTtccaaaaccatcgttctttcggtcggcgaggctactc
gcactctgactgagatccagtccaccgcagaccgtcagatcttcgaagagaaggtcgggcctct
ggtgggtcggctgcgcctcacggcttcgctccgtcaaaacggagccaagaccgcgtatcgcgtc
aacctaaaactggatcaggcggacgtcgttgattgctccaccagcgtctgcggcgagcttccga
aagtgcgctacactcaggtatggtcgcacgacgtgacaatcgttgcgaatagcaccgaggcctc
gcgcaaatcgttgtacgatttgaccaagtccctcgtcgcgacctcgcaggtcgaagatcttgtc
gtcaaccttgtgccgctgggccgtGGCGGAGGTGGTTCTaagctgaaccctcctgatgagagtg
gccccggctgcatgagctgctgtgtgctctcctaagaattcgatatcaagcttatcgataatca
acctctggattacaaaatttgtgaaagattgactggtattcttaactatgttgctccttttacg
ctatgtggatacgctgctttaatgcctttgtatcatgctattgcttcccgtatggctttcattt
tctcctccttgtataaatcctggttgctgtctctttatgaggagttgtggcccgttgtcaggca
acgtggcgtggtgtgcactgtgtttgctgacgcaacccccactggttggggcattgccaccacc
tgtcagctcctttccgggactttcgctttccccctccctattgccacggcggaactcatcgccg
cctgccttgcccgctgctggacaggggctcggctgttgggcactgacaattccgtggtgttgtc
ggggaaatcatcgtcctttccttggctgctcgcctatgttgccacctggattctgcgcgggacg
tccttctgctacgtcccttcggccctcaatccagcggaccttccttcccgcggcctgctgccgg
ctctgcggcctcttccgcgtcttcgccttcgccctcagacgagtcggatctccctttgggccgc
ctccccgcatcgataccgagcgctgctcgagagatctacgggtggcatccctgtgacccctccc
cagtgcctctcctggccctggaagttgccactccagtgcccaccagccttgtcctaataaaatt
aagttgcatcattttgtctgactaggtgtccttctataatattatggggtggaggggggtggta
tggagcaaggggcaagttgggaagacaacctgtagggcctgcggggtctattgggaaccaagct
ggagtgcagtggcacaatcttggctcactgcaatctccgcctcctgggttcaagcgattctcct
gcctcagcctcccgagttgttgggattccaggcatgcatgaccaggctcagctaatttttgttt
ttttggtagagacggggtttcaccatattggccaggctggtctccaactcctaatctcaggtga
tctacccaccttggcctcccaaattgctgggattacaggcgtgaaccactgctcccttccctgt
ccttctgattttgtaggtaaccacgtgcggaccgagcggccgcaggaacccctagtgatggagt
tggccactccctctctgcgcgctcgctcgctcactgaggccgggcgaccaaaggtcgcccgacg
cccgggctttgcccgggcggcctcagtgagcgagcgagcgcgcagctgcctgcaggggcgcctg
atgcggtattttctccttacgcatctgtgcggtatttcacaccgcatacgtcaaagcaaccata
gtacgcgccctgtagcggcgcattaagcgcggcgggtgtggtggttacgcgcagcgtgaccgct
acacttgccagcgccttagcgcccgctcctttcgctttcttcccttcctttctcgccacgttcg
ccggctttccccgtcaagctctaaatcgggggctccctttagggttccgatttagtgctttacg
gcacctcgaccccaaaaaacttgatttgggtgatggttcacgtagtgggccatcgccctgatag
acggtttttcgccctttgacgttggagtccacgttctttaatagtggactcttgttccaaactg
gaacaacactcaactctatctcgggctattcttttgatttataagggattttgccgatttcggt
ctattggttaaaaaatgagctgatttaacaaaaatttaacgcgaattttaacaaaatattaacg
tttacaattttatggtgcactctcagtacaatctgctctgatgccgcatagttaagccagcccc
gacacccgccaacacccgctgacgcgccctgacgggcttgtctgctcccggcatccgcttacag
acaagctgtgaccgtctccgggagctgcatgtgtcagaggttttcaccgtcatcaccgaaacgc
gcgagacgaaagggcctcgtgatacgcctatttttataggttaatgtcatgataataatggttt
cttagacgtcaggtggcacttttcggggaaatgtgcgcggaacccctatttgtttatttttcta
aatacattcaaatatgtatccgctcatgagacaataaccctgataaatgcttcaataatattga
aaaaggaagagtatgagtattcaacatttccgtgtcgcccttattcccttttttgcggcatttt
gccttcctgtttttgctcacccagaaacgctggtgaaagtaaaagatgctgaagatcagttggg
tgcacgagtgggttacatcgaactggatctcaacagcggtaagatccttgagagttttcgcccc
gaagaacgttttccaatgatgagcacttttaaagttctgctatgtggcgcggtattatcccgta
ttgacgccgggcaagagcaactcggtcgccgcatacactattctcagaatgacttggttgagta
ctcaccagtcacagaaaagcatcttacggatggcatgacagtaagagaattatgcagtgctgcc
ataaccatgagtgataacactgcggccaacttacttctgacaacgatcggaggaccgaaggagc
taaccgcttttttgcacaacatgggggatcatgtaactcgccttgatcgttgggaaccggagct
gaatgaagccataccaaacgacgagcgtgacaccacgatgcctgtagcaatggcaacaacgttg
cgcaaactattaactggcgaactacttactctagcttcccggcaacaattaatagactggatgg
aggcggataaagttgcaggaccacttctgcgctcggcccttccggctggctggtttattgctga
taaatctggagccggtgagcgtgggtctcgcggtatcattgcagcactggggccagatggtaag
ccctcccgtatcgtagttatctacacgacggggagtcaggcaactatggatgaacgaaatagac
agatcgctgagataggtgcctcactgattaagcattggtaactgtcagaccaagtttactcata
tatactttagattgatttaaaacttcatttttaatttaaaaggatctaggtgaagatccttttt
gataatctcatgaccaaaatcccttaacgtgagttttcgttccactgagcgtcagaccccgtag
aaaagatcaaaggatcttcttgagatcctttttttctgcgcgtaatctgctgcttgcaaacaaa
aaaaccaccgctaccagcggtggtttgtttgccggatcaagagctaccaactctttttccgaag
gtaactggcttcagcagagcgcagataccaaatactgttcttctagtgtagccgtagttaggcc
accacttcaagaactctgtagcaccgcctacatacctcgctctgctaatcctgttaccagtggc
tgctgccagtggcgataagtcgtgtcttaccgggttggactcaagacgatagttaccggataag
gcgcagcggtcgggctgaacggggggttcgtgcacacagcccagcttggagcgaacgacctaca
ccgaactgagatacctacagcgtgagctatgagaaagcgccacgcttcccgaagggagaaaggc
ggacaggtatccggtaagcggcagggtcggaacaggagagcgcacgagggagcttccaggggga
aacgcctggtatctttatagtcctgtcgggtttcgccacctctgacttgagcgtcgatttttgt
gatgctcgtcaggggggcggagcctatggaaaaacgccagcaacgcggcctttttacggttcct
ggccttttgctggccttttgctcacatgt

Plasmid #14 (see FIG. 32 for a map of the plasmid)

(SEQ ID NO: 107)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggccctgcgtatgagtgcaagtgggttttaggaccaggatgaggcgg
ggtgggggtgcctacctgacgaccgaccccgacccactggacaagcacccaacccccattcccc
aaattgcgcatcccctatcagagagggggaggggaaacaggatgcggcgaggcgcgtgcgcact
gccagcttcagcaccgcggacagtgccttcgcccccgcctggcggcgcgcgccaccgccgcctc
agcactgaaggcgcgctgacgtcactcgccggtcccccgcaaactccccttcccggccaccttg
gtcgcgtccgcgccgccgccggcccagccggaccgcaccacgcgaggcgcgagataggggggca
cgggcgcgaccatctgcgctgcggcgccggcgactcagcgctgcctcagtctgcggtgggcagc
ggaggagtcgtgtcgtgcctgagagcgcagtcgagaaggtaccGGATCCGCCACCatgggcaag
cccatccccaaccccctgctgggcctggacagcaccGGCGGTGGAGGTTCCtccaaaaccatcg
ttctttcggtcggcgaggctactcgcactctgactgagatccagtccaccgcagaccgtcagat
cttcgaagagaaggtcgggcctctggtgggtcggctgcgcctcacggcttcgctccgtcaaaac
ggagccaagaccgcgtatcgcgtcaacctaaaactggatcaggcggacgtcgttgattgctcca
ccagcgtctgcggcgagcttccgaaagtgcgctacactcaggtatggtcgcacgacgtgacaat
cgttgcgaatagcaccgaggcctcgcgcaaatcgttgtacgatttgaccaagtccctcgtcgcg
acctcgcaggtcgaagatcttgtcgtcaaccttgtgccgctgggccgtggcggtggcggatctg
gcggcggtggtagcAATGATTTTGGCAATTACAACAATCAGTCTTCCAATTTTGGGCCGATGAA
GGGAGGAAACTTTGGAGGCAGGAGCTCTGGCCCTTATGGTGGTGGAGGCCAGTACTTTGCTAAA
CCACGGAACCAAGGTGGCTATGGCGGAGGTGGTTCTctgcctccacttgaaagactgacactgt
aagaattcgatatcaagcttatcgataatcaacctctggattacaaaatttgtgaaagattgac
tggtattcttaactatgttgctccttttacgctatgtggatacgctgctttaatgcctttgtat
catgctattgcttcccgtatggctttcattttctcctccttgtataaatcctggttgctgtctc
tttatgaggagttgtggcccgttgtcaggcaacgtggcgtggtgtgcactgtgtttgctgacgc
aacccccactggttggggcattgccaccacctgtcagctcctttccgggactttcgctttcccc
ctccctattgccacggcggaactcatcgccgcctgccttgcccgctgctggacaggggctcggc
tgttgggcactgacaattccgtggtgttgtcggggaaatcatcgtcctttccttggctgctcgc
ctatgttgccacctggattctgcgcgggacgtccttctgctacgtcccttcggccctcaatcca
gcggaccttccttcccgcggcctgctgccggctctgcggcctcttccgcgtcttcgccttcgcc
ctcagacgagtcggatctccctttgggccgcctccccgcatcgataccgagcgctgctcgagag
atctacgggtggcatccctgtgacccctccccagtgcctctcctggccctggaagttgccactc
cagtgcccaccagccttgtcctaataaaattaagttgcatcattttgtctgactaggtgtcctt
ctataatattatggggtggaggggggtggtatggagcaaggggcaagttgggaagacaacctgt
agggcctgcggggtctattgggaaccaagctggagtgcagtggcacaatcttggctcactgcaa
tctccgcctcctgggttcaagcgattctcctgcctcagcctcccgagttgttgggattccaggc
atgcatgaccaggctcagctaatttttgtttttttggtagagacggggtttcaccatattggcc
aggctggtctccaactcctaatctcaggtgatctacccaccttggcctcccaaattgctgggat
tacaggcgtgaaccactgctcccttccctgtccttctgattttgtaggtaaccacgtgcggacc
gagcggccgcaggaacccctagtgatggagttggccactccctctctgcgcgctcgctcgctca
ctgaggccgggcgaccaaaggtcgcccgacgcccgggctttgcccgggcggcctcagtgagcga
gcgagcgcgcagctgcctgcaggggcgcctgatgcggtattttctccttacgcatctgtgcggt
atttcacaccgcatacgtcaaagcaaccatagtacgcgccctgtagcggcgcattaagcgcggc
gggtgtggtggttacgcgcagcgtgaccgctacacttgccagcgccttagcgcccgctcctttc
gctttcttcccttcctttctcgccacgttcgccggctttccccgtcaagctctaaatcgggggc
tccctttagggttccgatttagtgctttacggcacctcgaccccaaaaaacttgatttgggtga
tggttcacgtagtgggccatcgccctgatagacggtttttcgccctttgacgttggagtccacg
ttctttaatagtggactcttgttccaaactggaacaacactcaactctatctcgggctattctt
ttgatttataagggattttgccgatttcggtctattggttaaaaaatgagctgatttaacaaaa
atttaacgcgaattttaacaaaatattaacgtttacaattttatggtgcactctcagtacaatc
tgctctgatgccgcatagttaagccagccccgacacccgccaacacccgctgacgcgccctgac
gggcttgtctgctcccggcatccgcttacagacaagctgtgaccgtctccgggagctgcatgtg
tcagaggttttcaccgtcatcaccgaaacgcgcgagacgaaagggcctcgtgatacgcctattt
ttataggttaatgtcatgataataatggtttcttagacgtcaggtggcacttttcggggaaatg
tgcgcggaacccctatttgtttatttttctaaatacattcaaatatgtatccgctcatgagaca
ataaccctgataaatgcttcaataatattgaaaaaggaagagtatgagtattcaacatttccgt
gtcgcccttattcccttttttgcggcattttgccttcctgtttttgctcacccagaaacgctgg
tgaaagtaaaagatgctgaagatcagttgggtgcacgagtgggttacatcgaactggatctcaa
cagcggtaagatccttgagagttttcgccccgaagaacgttttccaatgatgagcacttttaaa
gttctgctatgtggcgcggtattatcccgtattgacgccgggcaagagcaactcggtcgccgca
tacactattctcagaatgacttggttgagtactcaccagtcacagaaaagcatcttacggatgg
catgacagtaagagaattatgcagtgctgccataaccatgagtgataacactgcggccaactta
cttctgacaacgatcggaggaccgaaggagctaaccgcttttttgcacaacatgggggatcatg
taactcgccttgatcgttgggaaccggagctgaatgaagccataccaaacgacgagcgtgacac
cacgatgcctgtagcaatggcaacaacgttgcgcaaactattaactggcgaactacttactcta
gcttcccggcaacaattaatagactggatggaggcggataaagttgcaggaccacttctgcgct
cggcccttccggctggctggtttattgctgataaatctggagccggtgagcgtgggtctcgcgg
tatcattgcagcactggggccagatggtaagccctcccgtatcgtagttatctacacgacgggg
agtcaggcaactatggatgaacgaaatagacagatcgctgagataggtgcctcactgattaagc
attggtaactgtcagaccaagtttactcatatatactttagattgatttaaaacttcattttta
atttaaaaggatctaggtgaagatcctttttgataatctcatgaccaaaatcccttaacgtgag
ttttcgttccactgagcgtcagaccccgtagaaaagatcaaaggatcttcttgagatccttttt
ttctgcgcgtaatctgctgcttgcaaacaaaaaaaccaccgctaccagcggtggtttgtttgcc
ggatcaagagctaccaactctttttccgaaggtaactggcttcagcagagcgcagataccaaat
actgttcttctagtgtagccgtagttaggccaccacttcaagaactctgtagcaccgcctacat
acctcgctctgctaatcctgttaccagtggctgctgccagtggcgataagtcgtgtcttaccgg
gttggactcaagacgatagttaccggataaggcgcagcggtcgggctgaacggggggttcgtgc
acacagcccagcttggagcgaacgacctacaccgaactgagatacctacagcgtgagctatgag
aaagcgccacgcttcccgaagggagaaaggcggacaggtatccggtaagcggcagggtcggaac
aggagagcgcacgagggagcttccagggggaaacgcctggtatctttatagtcctgtcgggttt
cgccacctctgacttgagcgtcgatttttgtgatgctcgtcaggggggcggagcctatggaaaa
acgccagcaacgcggcctttttacggttcctggccttttgctggccttttgctcacatgt

Plasmid #15 (see FIG. 33 for a map of the plasmid)

(SEQ ID NO: 108)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggccctgcgtatgagtgcaagtgggttttaggaccaggatgaggcgg
ggtgggggtgcctacctgacgaccgaccccgacccactggacaagcacccaacccccattcccc
aaattgcgcatcccctatcagagagggggaggggaaacaggatgcggcgaggcgcgtgcgcact
gccagcttcagcaccgcggacagtgccttcgcccccgcctggcggcgcgcgccaccgccgcctc
agcactgaaggcgcgctgacgtcactcgccggtcccccgcaaactccccttcccggccaccttg
gtcgcgtccgcgccgccgccggcccagccggaccgcaccacgcgaggcgcgagataggggggca
cgggcgcgaccatctgcgctgcggcgccggcgactcagcgctgcctcagtctgcggtgggcagc
ggaggagtcgtgtcgtgcctgagagcgcagtcgagaaggtaccGGATCCGCCACCatgggcaag
cccatccccaaccccctgctgggcctggacagcaccggcagcggcAACTATGAGCTTTTGACCA
CTGAGAACGCTCCTGTTAAGATGTGGACAAAAGGCGTGCCTGTAGAGGCCGACGCTCGGCAGCA
ACTCATTAACACCGCCAAGATGCCCTTTATTTTCAAGCATATTGCCGTGATGCCTGATGTCCAT
CTTGGTAAGGGTTCAACAATCGGGAGCGTCATCCCTACCAAGGGTGCCATCATTCCAGCCGCCG
TAGGAGTAGATATTGGATGCGGCATGAACGCACTTAGAACAGCTCTGACCGCCGAGGATCTTCC
CGAGAACCTCGCTGAACTGCGACAGGCAATCGAGACAGCAGTTCCTCACGGCAGAACCACAGGC
AGGTGTAAGAGAGATAAGGGCGCATGGGAAAACCCCCCCGTGAATGTCGACGCAAAATGGGCAG
AGTTGGAAGCTGGGTATCAATGGCTGACCCAAAAGTACCCACGGTTCCTCAATACTAATAACTA
TAAGCACCTTGGGACACTCGGAACCGGCAACCACTTCATAGAAATATGCCTGGACGAGTCAGAT
CAAGTTTGGATAATGCTCCACTCTGGTTCACGGGGCATTGGCAACGCTATAGGAACATACTTTA
TAGACCTGGCCCAGAAAGAGATGCAAGAAACATTGGAAACTCTCCCAAGTAGGGACCTCGCTTA
CTTCATGGAGGGAACTGAGTATTTCGATGATTATCTGAAAGCCGTAGCATGGGCACAGTTGTTC
GCCTCCTTGAATAGGGATGCAATGATGGAGAATGTCGTCACTGCTCTTCAAAGTATCACCCAAA
AAACAGTACGCCAACCTCAGACTCTGGCAATGGAAGAGATCAACTGTCATCATAACTACGTACA
AAAGGAACAACACTTCGGCGAAGAGATCTATGTTACCCGGAAAGGGGCCGTCTCAGCTAGGGCA
GGCCAATACGGCATAATCCCTGGCTCTATGGGTGCAAAAAGCTTCATAGTTCGAGGCCTTGGGA
ACGAGGAGAGCTTTTGTAGCTGTAGCCACGGGGCTGGTCGGGTGATGTCCCGGACTAAAGCTAA
AAAATTGTTCTCTGTTGAGGACCAAATACGGGCTACCGCACACGTAGAATGCCGGAAGGACGCC
GAGGTCATCGACGAAATCCCTATGGCCTACAAGGACATTGACGCAGTTATGGCCGCACAGTCTG
ACCTGGTGGAAGTTATATATACACTGAGGCAAGTAGTATGTGTGAAGGGAtctggtggttctcc
caagaagaagaggaaggtggaccccaagaagaagaggaaggtggaccccaagaagaagaggaag
gtgggctcaggaggagagggcagaggaagtcttctaacatgcggtgacgtggaggagaatcccg
gccctgCCatggattacaaggatgacgacgataagGGCGGAGGTGGTTCTtccaaaaccatcgt
tctttcggtcggcgaggctactcgcactctgactgagatccagtccaccgcagaccgtcagatc
ttcgaagagaaggtcgggcctctggtgggtcggctgcgcctcacggcttcgctccgtcaaaacg
gagccaagaccgcgtatcgcgtcaacctaaaactggatcaggcggacgtcgttgattgctccac
cagcgtctgcggcgagcttccgaaagtgcgctacactcaggtatggtcgcacgacgtgacaatc
gttgcgaatagcaccgaggcctcgcgcaaatcgttgtacgatttgaccaagtccctcgtcgcga
cctcgcaggtcgaagatcttgtcgtcaaccttgtgccgctgggccgtGGCGGAGGTGGTTCTaa
gctgaaccctcctgatgagagtggccccggctgcatgagctgctgtgtgctctcctaagaattc
gatatcaagcttatcgataatcaacctctggattacaaaatttgtgaaagattgactggtattc
ttaactatgttgctccttttacgctatgtggatacgctgctttaatgcctttgtatcatgctat
tgcttcccgtatggctttcattttctcctccttgtataaatcctggttgctgtctctttatgag
gagttgtggcccgttgtcaggcaacgtggcgtggtgtgcactgtgtttgctgacgcaaccccca
ctggttggggcattgccaccacctgtcagctcctttccgggactttcgctttccccctccctat
tgccacggcggaactcatcgccgcctgccttgcccgctgctggacaggggctcggctgttgggc
actgacaattccgtggtgttgtcggggaaatcatcgtcctttccttggctgctcgcctatgttg
ccacctggattctgcgcgggacgtccttctgctacgtcccttcggccctcaatccagcggacct
tccttcccgcggcctgctgccggctctgcggcctcttccgcgtcttcgccttcgccctcagacg
agtcggatctccctttgggccgcctccccgcatcgataccgagcgctgctcgagagatctacgg
gtggcatccctgtgacccctccccagtgcctctcctggccctggaagttgccactccagtgccc
accagccttgtcctaataaaattaagttgcatcattttgtctgactaggtgtccttctataata
ttatggggtggaggggggtggtatggagcaaggggcaagttgggaagacaacctgtagggcctg
cggggtctattgggaaccaagctggagtgcagtggcacaatcttggctcactgcaatctccgcc
tcctgggttcaagcgattctcctgcctcagcctcccgagttgttgggattccaggcatgcatga
ccaggctcagctaatttttgtttttttggtagagacggggtttcaccatattggccaggctggt
ctccaactcctaatctcaggtgatctacccaccttggcctcccaaattgctgggattacaggcg
tgaaccactgctcccttccctgtccttctgattttgtaggtaaccacgtgcggaccgagcggcc
gcaggaacccctagtgatggagttggccactccctctctgcgcgctcgctcgctcactgaggcc
gggcgaccaaaggtcgcccgacgcccgggctttgcccgggcggcctcagtgagcgagcgagcgc
gcagctgcctgcaggggcgcctgatgcggtattttctccttacgcatctgtgcggtatttcaca
ccgcatacgtcaaagcaaccatagtacgcgccctgtagcggcgcattaagcgcggcgggtgtgg
tggttacgcgcagcgtgaccgctacacttgccagcgccttagcgcccgctcctttcgctttctt
cccttcctttctcgccacgttcgccggctttccccgtcaagctctaaatcgggggctcccttta
gggttccgatttagtgctttacggcacctcgaccccaaaaaacttgatttgggtgatggttcac
gtagtgggccatcgccctgatagacggtttttcgccctttgacgttggagtccacgttctttaa
tagtggactcttgttccaaactggaacaacactcaactctatctcgggctattcttttgattta
taagggattttgccgatttcggtctattggttaaaaaatgagctgatttaacaaaaatttaacg
cgaattttaacaaaatattaacgtttacaattttatggtgcactctcagtacaatctgctctga
tgccgcatagttaagccagccccgacacccgccaacacccgctgacgcgccctgacgggcttgt
ctgctcccggcatccgcttacagacaagctgtgaccgtctccgggagctgcatgtgtcagaggt
tttcaccgtcatcaccgaaacgcgcgagacgaaagggcctcgtgatacgcctatttttataggt
taatgtcatgataataatggtttcttagacgtcaggtggcacttttcggggaaatgtgcgcgga
acccctatttgtttatttttctaaatacattcaaatatgtatccgctcatgagacaataaccct
gataaatgcttcaataatattgaaaaaggaagagtatgagtattcaacatttccgtgtcgccct
tattcccttttttgcggcattttgccttcctgtttttgctcacccagaaacgctggtgaaagta
aaagatgctgaagatcagttgggtgcacgagtgggttacatcgaactggatctcaacagcggta
agatccttgagagttttcgccccgaagaacgttttccaatgatgagcacttttaaagttctgct
atgtggcgcggtattatcccgtattgacgccgggcaagagcaactcggtcgccgcatacactat
tctcagaatgacttggttgagtactcaccagtcacagaaaagcatcttacggatggcatgacag
taagagaattatgcagtgctgccataaccatgagtgataacactgcggccaacttacttctgac
aacgatcggaggaccgaaggagctaaccgcttttttgcacaacatgggggatcatgtaactcgc
cttgatcgttgggaaccggagctgaatgaagccataccaaacgacgagcgtgacaccacgatgc
ctgtagcaatggcaacaacgttgcgcaaactattaactggcgaactacttactctagcttcccg
gcaacaattaatagactggatggaggcggataaagttgcaggaccacttctgcgctcggccctt
ccggctggctggtttattgctgataaatctggagccggtgagcgtgggtctcgcggtatcattg
cagcactggggccagatggtaagccctcccgtatcgtagttatctacacgacggggagtcaggc
aactatggatgaacgaaatagacagatcgctgagataggtgcctcactgattaagcattggtaa
ctgtcagaccaagtttactcatatatactttagattgatttaaaacttcatttttaatttaaaa
ggatctaggtgaagatcctttttgataatctcatgaccaaaatcccttaacgtgagttttcgtt
ccactgagcgtcagaccccgtagaaaagatcaaaggatcttcttgagatcctttttttctgcgc
gtaatctgctgcttgcaaacaaaaaaaccaccgctaccagcggtggtttgtttgccggatcaag
agctaccaactctttttccgaaggtaactggcttcagcagagcgcagataccaaatactgttct
tctagtgtagccgtagttaggccaccacttcaagaactctgtagcaccgcctacatacctcgct
ctgctaatcctgttaccagtggctgctgccagtggcgataagtcgtgtcttaccgggttggact
caagacgatagttaccggataaggcgcagcggtcgggctgaacggggggttcgtgcacacagcc
cagcttggagcgaacgacctacaccgaactgagatacctacagcgtgagctatgagaaagcgcc
acgcttcccgaagggagaaaggcggacaggtatccggtaagcggcagggtcggaacaggagagc
gcacgagggagcttccagggggaaacgcctggtatctttatagtcctgtcgggtttcgccacct
ctgacttgagcgtcgatttttgtgatgctcgtcaggggggcggagcctatggaaaaacgccagc
aacgcggcctttttacggttcctggccttttgctggccttttgctcacatgt

Plasmid #16 (see FIG. 34 for a map of the plasmid)

(SEQ ID NO: 109)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggccctgcgtatgagtgcaagtgggttttaggaccaggatgaggcgg
ggtgggggtgcctacctgacgaccgaccccgacccactggacaagcacccaacccccattcccc
aaattgcgcatcccctatcagagagggggaggggaaacaggatgcggcgaggcgcgtgcgcact
gccagcttcagcaccgcggacagtgccttcgcccccgcctggcggcgcgcgccaccgccgcctc
agcactgaaggcgcgctgacgtcactcgccggtcccccgcaaactccccttcccggccaccttg
gtcgcgtccgcgccgccgccggcccagccggaccgcaccacgcgaggcgcgagataggggggca
cgggcgcgaccatctgcgctgcggcgccggcgactcagcgctgcctcagtctgcggtgggcagc
ggaggagtcgtgtcgtgcctgagagcgcagtcgagaaggtaccGGATCCGCCACCatgggcaag
cccatccccaaccccctgctgggcctggacagcaccggcagcggcGCAGAACAGGATGTGGAAA
ACGATCTTTTGGATTACGATGAAGAGGAAGAGCCCCAGGCTCCTCAAGAGAGCACACCAGCTCC
CCCTAAGAAAGACATCAAGGGATCCTACGTTTCCATCCACAGCTCTGGCTTCCGGGACTTTCTG
CTGAAGCCGGAGCTCCTGCGGGCCATCGTGGACTGTGGCTTTGAGCATCCTTCTGAGGTCCAGC
ATGAGTGCATTCCCCAGGCCATCCTGGGCATGGACGTCCTGTGCCAGGCCAAGTCCGGGATGGG
CAAGACAGCGGTCTTCGTGCTGGCCACCCTACAGCAGATTGAGCCTGTCAACGGACAGGTGACG
GTCCTGGTCATGTGCCACACGAGGGAGCTGGCCTTCCAGATCAGCAAGGAATATGAGCGCTTTT
CCAAGTACATGCCCAGCGTCAAGGTGTCTGTGTTCTTCGGTGGTCTCTCCATCAAGAAGGATGA
AGAAGTGTTGAAGAAGAACTGTCCCCATGTCGTGGTGGGGACCCCGGGCCGCATCCTGGCGCTC
GTGCGGAATAGGAGCTTCAGCCTAAAGAATGTGAAGCACTTTGTGCTGGACGAGTGTGACAAGA
TGCTGGAGCAGCTGGACATGCGGCGGGATGTGCAGGAGATCTTCCGCCTGACACCACACGAGAA
GCAGTGCATGATGTTCAGCGCCACCCTGAGCAAGGACATCCGGCCTGTGTGCAGGAAGTTCATG
CAGGATCCAATGGAGGTGTTTGTGGACGACGAGACCAAGCTCACGCTGCACGGCCTGCAGCAGT
ACTACGTCAAACTCAAAGACAGTGAGAAGAACCGCAAGCTCTTTGATCTCTTGGATGTGCTGGA
GTTTAACCAGGTGATAATCTTCGTCAAGTCAGTGCAGCGCTGCATGGCCCTGGCCCAGCTCCTC
GTGGAGCAGAACTTCCCGGCCATCGCCATCCACCGGGGCATGGCCCAGGAGGAGCGCCTGTCAC
GCTATCAGCAGTTCAAGGATTTCCAGCGGCGGATCCTGGTGGCCACCAATCTGTTTGGCCGGGG
GATGGACATCGAGCGAGTCAACATCGTCTTTAACTACGACATGCCTGAGGACTCGGACACCTAC
CTGCACCGGGTGGCCCGGGCGGGTCGCTTTGGCACCAAAGGCCTAGCCATCACTTTTGTGTCTG
ACGAGAATGATGCCAAAATCCTCAATGACGTCCAGGACCGGTTTGAAGTTAATGTGGCAGAACT
TCCAGAGGAAATCGACATCTCCACATACATCGAGCAGAGCCGGtctggtggttctgagggcaga
ggaagtcttctaacatgcggtgacgtggaggagaatcccggccctgCCatggattacaaggatg
acgacgataagGGCGGAGGTGGTTCTtccaaaaccatcgttctttcggtcggcgaggctactcg
cactctgactgagatccagtccaccgcagaccgtcagatcttcgaagagaaggtcgggcctctg
gtgggtcggctgcgcctcacggcttcgctccgtcaaaacggagccaagaccgcgtatcgcgtca
acctaaaactggatcaggcggacgtcgttgattgctccaccagcgtctgcggcgagcttccgaa
agtgcgctacactcaggtatggtcgcacgacgtgacaatcgttgcgaatagcaccgaggcctcg
cgcaaatcgttgtacgatttgaccaagtccctcgtcgcgacctcgcaggtcgaagatcttgtcg
tcaaccttgtgccgctgggccgtGGCGGAGGTGGTTCTaagctgaaccctcctgatgagagtgg
ccccggctgcatgagctgctgtgtgctctcctaagaattcgatatcaagcttatcgataatcaa
cctctggattacaaaatttgtgaaagattgactggtattcttaactatgttgctccttttacgc
tatgtggatacgctgctttaatgcctttgtatcatgctattgcttcccgtatggctttcatttt
ctcctccttgtataaatcctggttgctgtctctttatgaggagttgtggcccgttgtcaggcaa
cgtggcgtggtgtgcactgtgtttgctgacgcaacccccactggttggggcattgccaccacct
gtcagctcctttccgggactttcgctttccccctccctattgccacggcggaactcatcgccgc
ctgccttgcccgctgctggacaggggctcggctgttgggcactgacaattccgtggtgttgtcg
gggaaatcatcgtcctttccttggctgctcgcctatgttgccacctggattctgcgcgggacgt
ccttctgctacgtcccttcggccctcaatccagcggaccttccttcccgcggcctgctgccggc
tctgcggcctcttccgcgtcttcgccttcgccctcagacgagtcggatctccctttgggccgcc
tccccgcatcgataccgagcgctgctcgagagatctacgggtggcatccctgtgacccctcccc
agtgcctctcctggccctggaagttgccactccagtgcccaccagccttgtcctaataaaatta
agttgcatcattttgtctgactaggtgtccttctataatattatggggtggaggggggtggtat
ggagcaaggggcaagttgggaagacaacctgtagggcctgcggggtctattgggaaccaagctg
gagtgcagtggcacaatcttggctcactgcaatctccgcctcctgggttcaagcgattctcctg
cctcagcctcccgagttgttgggattccaggcatgcatgaccaggctcagctaatttttgtttt
tttggtagagacggggtttcaccatattggccaggctggtctccaactcctaatctcaggtgat
ctacccaccttggcctcccaaattgctgggattacaggcgtgaaccactgctcccttccctgtc
cttctgattttgtaggtaaccacgtgcggaccgagcggccgcaggaacccctagtgatggagtt
ggccactccctctctgcgcgctcgctcgctcactgaggccgggcgaccaaaggtcgcccgacgc
ccgggctttgcccgggcggcctcagtgagcgagcgagcgcgcagctgcctgcaggggcgcctga
tgcggtattttctccttacgcatctgtgcggtatttcacaccgcatacgtcaaagcaaccatag
tacgcgccctgtagcggcgcattaagcgcggcgggtgtggtggttacgcgcagcgtgaccgcta
cacttgccagcgccttagcgcccgctcctttcgctttcttcccttcctttctcgccacgttcgc
cggctttccccgtcaagctctaaatcgggggctccctttagggttccgatttagtgctttacgg
cacctcgaccccaaaaaacttgatttgggtgatggttcacgtagtgggccatcgccctgataga
cggtttttcgccctttgacgttggagtccacgttctttaatagtggactcttgttccaaactgg
aacaacactcaactctatctcgggctattcttttgatttataagggattttgccgatttcggtc
tattggttaaaaaatgagctgatttaacaaaaatttaacgcgaattttaacaaaatattaacgt
ttacaattttatggtgcactctcagtacaatctgctctgatgccgcatagttaagccagccccg
acacccgccaacacccgctgacgcgccctgacgggcttgtctgctcccggcatccgcttacaga
caagctgtgaccgtctccgggagctgcatgtgtcagaggttttcaccgtcatcaccgaaacgcg
cgagacgaaagggcctcgtgatacgcctatttttataggttaatgtcatgataataatggtttc
ttagacgtcaggtggcacttttcggggaaatgtgcgcggaacccctatttgtttatttttctaa
atacattcaaatatgtatccgctcatgagacaataaccctgataaatgcttcaataatattgaa
aaaggaagagtatgagtattcaacatttccgtgtcgcccttattcccttttttgcggcattttg
ccttcctgtttttgctcacccagaaacgctggtgaaagtaaaagatgctgaagatcagttgggt
gcacgagtgggttacatcgaactggatctcaacagcggtaagatccttgagagttttcgccccg
aagaacgttttccaatgatgagcacttttaaagttctgctatgtggcgcggtattatcccgtat
tgacgccgggcaagagcaactcggtcgccgcatacactattctcagaatgacttggttgagtac
tcaccagtcacagaaaagcatcttacggatggcatgacagtaagagaattatgcagtgctgcca
taaccatgagtgataacactgcggccaacttacttctgacaacgatcggaggaccgaaggagct
aaccgcttttttgcacaacatgggggatcatgtaactcgccttgatcgttgggaaccggagctg
aatgaagccataccaaacgacgagcgtgacaccacgatgcctgtagcaatggcaacaacgttgc
gcaaactattaactggcgaactacttactctagcttcccggcaacaattaatagactggatgga
ggcggataaagttgcaggaccacttctgcgctcggcccttccggctggctggtttattgctgat
aaatctggagccggtgagcgtgggtctcgcggtatcattgcagcactggggccagatggtaagc
cctcccgtatcgtagttatctacacgacggggagtcaggcaactatggatgaacgaaatagaca
gatcgctgagataggtgcctcactgattaagcattggtaactgtcagaccaagtttactcatat
atactttagattgatttaaaacttcatttttaatttaaaaggatctaggtgaagatcctttttg
ataatctcatgaccaaaatcccttaacgtgagttttcgttccactgagcgtcagaccccgtaga
aaagatcaaaggatcttcttgagatcctttttttctgcgcgtaatctgctgcttgcaaacaaaa
aaaccaccgctaccagcggtggtttgtttgccggatcaagagctaccaactctttttccgaagg
taactggcttcagcagagcgcagataccaaatactgttcttctagtgtagccgtagttaggcca
ccacttcaagaactctgtagcaccgcctacatacctcgctctgctaatcctgttaccagtggct
gctgccagtggcgataagtcgtgtcttaccgggttggactcaagacgatagttaccggataagg
cgcagcggtcgggctgaacggggggttcgtgcacacagcccagcttggagcgaacgacctacac
cgaactgagatacctacagcgtgagctatgagaaagcgccacgcttcccgaagggagaaaggcg
gacaggtatccggtaagcggcagggtcggaacaggagagcgcacgagggagcttccagggggaa
acgcctggtatctttatagtcctgtcgggtttcgccacctctgacttgagcgtcgatttttgtg
atgctcgtcaggggggcggagcctatggaaaaacgccagcaacgcggcctttttacggttcctg
gccttttgctggccttttgctcacatgt

Plasmid #22 (see FIG. 48 for a map of the plasmid)

(SEQ ID NO: 86)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgcttaagaaaaaaaaaggggttggggatttagctcagtggtagagcgcttgcc
tagcaagcgcaaggccctgggttcggtcctcagctctggaaaaaaaaaaaaaaaaaaaaaaaga
caaaataacaaaaagaccaaaaaaaaacaaggtaactggcacacacaacctttaaaaaaaaagt
taaccggtccgatactctgatgatgggtcccCCTAGGTTAAGGATGCACCGACGGGACGTTCTA
TGAGGACGAATCTCCCGCTTATAAGATTCTATAAACTGTGCGGTCCTTCAATTGgggtcccatc
attcatggcaagtggccgcggtcggcgtggactgtagaacactgccaatgccggtcccaagccc
ggataaaaGTGGAGGGTACAGTCCACGCtttttttctagactgcagagggccctgcgtatgagt
gcaagtgggttttaggaccaggatgaggcggggtgggggtgcctacctgacgaccgaccccgac
ccactggacaagcacccaacccccattccccaaattgcgcatcccctatcagagagggggaggg
gaaacaggatgcggcgaggcgcgtgcgcactgccagcttcagcaccgcggacagtgccttcgcc
cccgcctggcggcgcgcgccaccgccgcctcagcactgaaggcgcgctgacgtcactcgccggt
cccccgcaaactccccttcccggccaccttggtcgcgtccgcgccgccgccggcccagccggac
cgcaccacgcgaggcgcgagataggggggcacgggcgcgaccatctgcgctgcggcgccggcga
ctcagcgctgcctcagtctgcggtgggcagcggaggagtcgtgtcgtgcctgagagcgcagtcg
agaaggtaccggatccgtgagcaagggcgaggaggataacatggccatcatcaaggagttcatg
cgcttcaaggtgcacatggagggctccgtgaacggccacgagttcgagatcgagggcgagggcg
agggccgcccctacgagggcacccagaccgccaagctgaaggtgaccaagggtggccccctgcc
cttcgcctgggacatcctgtcccctcagttcatgtacggctccaaggcctacgtgaagcacccc
gccgacatccccgactacttgaagctgtccttccccgagggcttcaagtgggagcgcgtgatga
acttcgaggacggcggcgtggtgaccgtgacccaggactcctccctgcaggacggcgagttcat
ctacaaggtgaagctgcgcggcaccaacttcccctccgacggccccgtaatgcagaagaagacc
atgggctgggaggcctcctccgagcggatgtaccccgaggacggcgccctgaagggcgagatca
agcagaggctgaagctgaaggacggcggccactacgacgctgaggtcaagaccacctacaaggc
caagaagcccgtgcagctgcccggcgcctacaacgtcaacatcaagttggacatcacctcccac
aacgaggactacaccatcgtggaacagtacgaacgcgccgagggccgccactccaccggcggca
tggacgagctgtacaagtaagaattcgatatcaagcttatcgataatcaacctctggattacaa
aatttgtgaaagattgactggtattcttaactatgttgctccttttacgctatgtggatacgct
gctttaatgcctttgtatcatgctattgcttcccgtatggctttcattttctcctccttgtata
aatcctggttgctgtctctttatgaggagttgtggcccgttgtcaggcaacgtggcgtggtgtg
cactgtgtttgctgacgcaacccccactggttggggcattgccaccacctgtcagctcctttcc
gggactttcgctttccccctccctattgccacggcggaactcatcgccgcctgccttgcccgct
gctggacaggggctcggctgttgggcactgacaattccgtggtgttgtcggggaaatcatcgtc
ctttccttggctgctcgcctatgttgccacctggattctgcgcgggacgtccttctgctacgtc
ccttcggccctcaatccagcggaccttccttcccgcggcctgctgccggctctgcggcctcttc
cgcgtcttcgccttcgccctcagacgagtcggatctccctttgggccgcctccccgcatcgata
ccgagcgctgctcgagagatctacgggtggcatccctgtgacccctccccagtgcctctcctgg
ccctggaagttgccactccagtgcccaccagccttgtcctaataaaattaagttgcatcatttt
gtctgactaggtgtccttctataatattatggggtggaggggggtggtatggagcaaggggcaa
gttgggaagacaacctgtagggcctgcggggtctattgggaaccaagctggagtgcagtggcac
aatcttggctcactgcaatctccgcctcctgggttcaagcgattctcctgcctcagcctcccga
gttgttgggattccaggcatgcatgaccaggctcagctaatttttgtttttttggtagagacgg
ggtttcaccatattggccaggctggtctccaactcctaatctcaggtgatctacccaccttggc
ctcccaaattgctgggattacaggcgtgaaccactgctcccttccctgtccttctgattttgta
ggtaaccacgtgcggaccgagcggccgcaggaacccctagtgatggagttggccactccctctc
tgcgcgctcgctcgctcactgaggccgggcgaccaaaggtcgcccgacgcccgggctttgcccg
ggcggcctcagtgagcgagcgagcgcgcagctgcctgcaggggcgcctgatgcggtattttctc
cttacgcatctgtgcggtatttcacaccgcatacgtcaaagcaaccatagtacgcgccctgtag
cggcgcattaagcgcggcgggtgtggtggttacgcgcagcgtgaccgctacacttgccagcgcc
ttagcgcccgctcctttcgctttcttcccttcctttctcgccacgttcgccggctttccccgtc
aagctctaaatcgggggctccctttagggttccgatttagtgctttacggcacctcgaccccaa
aaaacttgatttgggtgatggttcacgtagtgggccatcgccctgatagacggtttttcgccct
ttgacgttggagtccacgttctttaatagtggactcttgttccaaactggaacaacactcaact
ctatctcgggctattcttttgatttataagggattttgccgatttcggtctattggttaaaaaa
tgagctgatttaacaaaaatttaacgcgaattttaacaaaatattaacgtttacaattttatgg
tgcactctcagtacaatctgctctgatgccgcatagttaagccagccccgacacccgccaacac
ccgctgacgcgccctgacgggcttgtctgctcccggcatccgcttacagacaagctgtgaccgt
ctccgggagctgcatgtgtcagaggttttcaccgtcatcaccgaaacgcgcgagacgaaagggc
ctcgtgatacgcctatttttataggttaatgtcatgataataatggtttcttagacgtcaggtg
gcacttttcggggaaatgtgcgcggaacccctatttgtttatttttctaaatacattcaaatat
gtatccgctcatgagacaataaccctgataaatgcttcaataatattgaaaaaggaagagtatg
agtattcaacatttccgtgtcgcccttattcccttttttgcggcattttgccttcctgtttttg
ctcacccagaaacgctggtgaaagtaaaagatgctgaagatcagttgggtgcacgagtgggtta
catcgaactggatctcaacagcggtaagatccttgagagttttcgccccgaagaacgttttcca
atgatgagcacttttaaagttctgctatgtggcgcggtattatcccgtattgacgccgggcaag
agcaactcggtcgccgcatacactattctcagaatgacttggttgagtactcaccagtcacaga
aaagcatcttacggatggcatgacagtaagagaattatgcagtgctgccataaccatgagtgat
aacactgcggccaacttacttctgacaacgatcggaggaccgaaggagctaaccgcttttttgc
acaacatgggggatcatgtaactcgccttgatcgttgggaaccggagctgaatgaagccatacc
aaacgacgagcgtgacaccacgatgcctgtagcaatggcaacaacgttgcgcaaactattaact
ggcgaactacttactctagcttcccggcaacaattaatagactggatggaggcggataaagttg
caggaccacttctgcgctcggcccttccggctggctggtttattgctgataaatctggagccgg
tgagcgtgggtctcgcggtatcattgcagcactggggccagatggtaagccctcccgtatcgta
gttatctacacgacggggagtcaggcaactatggatgaacgaaatagacagatcgctgagatag
gtgcctcactgattaagcattggtaactgtcagaccaagtttactcatatatactttagattga
tttaaaacttcatttttaatttaaaaggatctaggtgaagatcctttttgataatctcatgacc
aaaatcccttaacgtgagttttcgttccactgagcgtcagaccccgtagaaaagatcaaaggat
cttcttgagatcctttttttctgcgcgtaatctgctgcttgcaaacaaaaaaaccaccgctacc
agcggtggtttgtttgccggatcaagagctaccaactctttttccgaaggtaactggcttcagc
agagcgcagataccaaatactgttcttctagtgtagccgtagttaggccaccacttcaagaact
ctgtagcaccgcctacatacctcgctctgctaatcctgttaccagtggctgctgccagtggcga
taagtcgtgtcttaccgggttggactcaagacgatagttaccggataaggcgcagcggtcgggc
tgaacggggggttcgtgcacacagcccagcttggagcgaacgacctacaccgaactgagatacc
tacagcgtgagctatgagaaagcgccacgcttcccgaagggagaaaggcggacaggtatccggt
aagcggcagggtcggaacaggagagcgcacgagggagcttccagggggaaacgcctggtatctt
tatagtcctgtcgggtttcgccacctctgacttgagcgtcgatttttgtgatgctcgtcagggg
ggcggagcctatggaaaaacgccagcaacgcggcctttttacggttcctggccttttgctggcc
ttttgctcacatgt

Plasmid #23 (see FIG. 36 for a map of the plasmid)

(SEQ ID NO: 110)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgcttaagaaaaaaaaaggccgggcgcggtggctcacgcctgtaatcccagctc
tcagggaggctaagaggcgggaggatagcttgagcccaggagttcgagacctgcctgggcaata
tagcgagaccccgttctccagaaaaaggaaaaaaaaaaacaaaagacaaaaaaaaaataagcgt
aacttccctcaaagcaacaacccccccccccctttaaaaaaaaagttaaccggtccgatactct
gatgatgggtcccCCTAGGTTAAGGATGCACCGACGGGACGTTCTATGAGGACGAATCTCCCGC
TTATAAGATTCTATAAACTGTGCGGTCCTTCAATTGgggtcccatcattcatggcaagtggccg
cggtcggcgtggactgtagaacactgccaatgccggtcccaagcccggataaaaGTGGAGGGTA
CAGTCCACGCtttttttctagactgcagagggccctgcgtatgagtgcaagtgggttttaggac
caggatgaggcggggtgggggtgcctacctgacgaccgaccccgacccactggacaagcaccca
acccccattccccaaattgcgcatcccctatcagagagggggaggggaaacaggatgcggcgag
gcgcgtgcgcactgccagcttcagcaccgcggacagtgccttcgcccccgcctggcggcgcgcg
ccaccgccgcctcagcactgaaggcgcgctgacgtcactcgccggtcccccgcaaactcccctt
cccggccaccttggtcgcgtccgcgccgccgccggcccagccggaccgcaccacgcgaggcgcg
agataggggggcacgggcgcgaccatctgcgctgcggcgccggcgactcagcgctgcctcagtc
tgcggtgggcagcggaggagtcgtgtcgtgcctgagagcgcagtcgagaaggtaccggatccgt
gagcaagggcgaggaggataacatggccatcatcaaggagttcatgcgcttcaaggtgcacatg
gagggctccgtgaacggccacgagttcgagatcgagggcgagggcgagggccgcccctacgagg
gcacccagaccgccaagctgaaggtgaccaagggtggccccctgcccttcgcctgggacatcct
gtcccctcagttcatgtacggctccaaggcctacgtgaagcaccccgccgacatccccgactac
ttgaagctgtccttccccgagggcttcaagtgggagcgcgtgatgaacttcgaggacggcggcg
tggtgaccgtgacccaggactcctccctgcaggacggcgagttcatctacaaggtgaagctgcg
cggcaccaacttcccctccgacggccccgtaatgcagaagaagaccatgggctgggaggcctcc
tccgagcggatgtaccccgaggacggcgccctgaagggcgagatcaagcagaggctgaagctga
aggacggcggccactacgacgctgaggtcaagaccacctacaaggccaagaagcccgtgcagct
gcccggcgcctacaacgtcaacatcaagttggacatcacctcccacaacgaggactacaccatc
gtggaacagtacgaacgcgccgagggccgccactccaccggcggcatggacgagctgtacaagt
aagaattcgatatcaagcttatcgataatcaacctctggattacaaaatttgtgaaagattgac
tggtattcttaactatgttgctccttttacgctatgtggatacgctgctttaatgcctttgtat
catgctattgcttcccgtatggctttcattttctcctccttgtataaatcctggttgctgtctc
tttatgaggagttgtggcccgttgtcaggcaacgtggcgtggtgtgcactgtgtttgctgacgc
aacccccactggttggggcattgccaccacctgtcagctcctttccgggactttcgctttcccc
ctccctattgccacggcggaactcatcgccgcctgccttgcccgctgctggacaggggctcggc
tgttgggcactgacaattccgtggtgttgtcggggaaatcatcgtcctttccttggctgctcgc
ctatgttgccacctggattctgcgcgggacgtccttctgctacgtcccttcggccctcaatcca
gcggaccttccttcccgcggcctgctgccggctctgcggcctcttccgcgtcttcgccttcgcc
ctcagacgagtcggatctccctttgggccgcctccccgcatcgataccgagcgctgctcgagag
atctacgggtggcatccctgtgacccctccccagtgcctctcctggccctggaagttgccactc
cagtgcccaccagccttgtcctaataaaattaagttgcatcattttgtctgactaggtgtcctt
ctataatattatggggtggaggggggtggtatggagcaaggggcaagttgggaagacaacctgt
agggcctgcggggtctattgggaaccaagctggagtgcagtggcacaatcttggctcactgcaa
tctccgcctcctgggttcaagcgattctcctgcctcagcctcccgagttgttgggattccaggc
atgcatgaccaggctcagctaatttttgtttttttggtagagacggggtttcaccatattggcc
aggctggtctccaactcctaatctcaggtgatctacccaccttggcctcccaaattgctgggat
tacaggcgtgaaccactgctcccttccctgtccttctgattttgtaggtaaccacgtgcggacc
gagcggccgcaggaacccctagtgatggagttggccactccctctctgcgcgctcgctcgctca
ctgaggccgggcgaccaaaggtcgcccgacgcccgggctttgcccgggcggcctcagtgagcga
gcgagcgcgcagctgcctgcaggggcgcctgatgcggtattttctccttacgcatctgtgcggt
atttcacaccgcatacgtcaaagcaaccatagtacgcgccctgtagcggcgcattaagcgcggc
gggtgtggtggttacgcgcagcgtgaccgctacacttgccagcgccttagcgcccgctcctttc
gctttcttcccttcctttctcgccacgttcgccggctttccccgtcaagctctaaatcgggggc
tccctttagggttccgatttagtgctttacggcacctcgaccccaaaaaacttgatttgggtga
tggttcacgtagtgggccatcgccctgatagacggtttttcgccctttgacgttggagtccacg
ttctttaatagtggactcttgttccaaactggaacaacactcaactctatctcgggctattctt
ttgatttataagggattttgccgatttcggtctattggttaaaaaatgagctgatttaacaaaa
atttaacgcgaattttaacaaaatattaacgtttacaattttatggtgcactctcagtacaatc
tgctctgatgccgcatagttaagccagccccgacacccgccaacacccgctgacgcgccctgac
gggcttgtctgctcccggcatccgcttacagacaagctgtgaccgtctccgggagctgcatgtg
tcagaggttttcaccgtcatcaccgaaacgcgcgagacgaaagggcctcgtgatacgcctattt
ttataggttaatgtcatgataataatggtttcttagacgtcaggtggcacttttcggggaaatg
tgcgcggaacccctatttgtttatttttctaaatacattcaaatatgtatccgctcatgagaca
ataaccctgataaatgcttcaataatattgaaaaaggaagagtatgagtattcaacatttccgt
gtcgcccttattcccttttttgcggcattttgccttcctgtttttgctcacccagaaacgctgg
tgaaagtaaaagatgctgaagatcagttgggtgcacgagtgggttacatcgaactggatctcaa
cagcggtaagatccttgagagttttcgccccgaagaacgttttccaatgatgagcacttttaaa
gttctgctatgtggcgcggtattatcccgtattgacgccgggcaagagcaactcggtcgccgca
tacactattctcagaatgacttggttgagtactcaccagtcacagaaaagcatcttacggatgg
catgacagtaagagaattatgcagtgctgccataaccatgagtgataacactgcggccaactta
cttctgacaacgatcggaggaccgaaggagctaaccgcttttttgcacaacatgggggatcatg
taactcgccttgatcgttgggaaccggagctgaatgaagccataccaaacgacgagcgtgacac
cacgatgcctgtagcaatggcaacaacgttgcgcaaactattaactggcgaactacttactcta
gcttcccggcaacaattaatagactggatggaggcggataaagttgcaggaccacttctgcgct
cggcccttccggctggctggtttattgctgataaatctggagccggtgagcgtgggtctcgcgg
tatcattgcagcactggggccagatggtaagccctcccgtatcgtagttatctacacgacgggg
agtcaggcaactatggatgaacgaaatagacagatcgctgagataggtgcctcactgattaagc
attggtaactgtcagaccaagtttactcatatatactttagattgatttaaaacttcattttta
atttaaaaggatctaggtgaagatcctttttgataatctcatgaccaaaatcccttaacgtgag
ttttcgttccactgagcgtcagaccccgtagaaaagatcaaaggatcttcttgagatccttttt
ttctgcgcgtaatctgctgcttgcaaacaaaaaaaccaccgctaccagcggtggtttgtttgcc
ggatcaagagctaccaactctttttccgaaggtaactggcttcagcagagcgcagataccaaat
actgttcttctagtgtagccgtagttaggccaccacttcaagaactctgtagcaccgcctacat
acctcgctctgctaatcctgttaccagtggctgctgccagtggcgataagtcgtgtcttaccgg
gttggactcaagacgatagttaccggataaggcgcagcggtcgggctgaacggggggttcgtgc
acacagcccagcttggagcgaacgacctacaccgaactgagatacctacagcgtgagctatgag
aaagcgccacgcttcccgaagggagaaaggcggacaggtatccggtaagcggcagggtcggaac
aggagagcgcacgagggagcttccagggggaaacgcctggtatctttatagtcctgtcgggttt
cgccacctctgacttgagcgtcgatttttgtgatgctcgtcaggggggcggagcctatggaaaa
acgccagcaacgcggcctttttacggttcctggccttttgctggccttttgctcacatgt

Plasmid #17 (see FIG. 37 for a map of the plasmid)

(SEQ ID NO: 111)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggccctgcgtatgagtgcaagtgggttttaggaccaggatgaggcgg
ggtgggggtgcctacctgacgaccgaccccgacccactggacaagcacccaacccccattcccc
aaattgcgcatcccctatcagagagggggaggggaaacaggatgcggcgaggcgcgtgcgcact
gccagcttcagcaccgcggacagtgccttcgcccccgcctggcggcgcgcgccaccgccgcctc
agcactgaaggcgcgctgacgtcactcgccggtcccccgcaaactccccttcccggccaccttg
gtcgcgtccgcgccgccgccggcccagccggaccgcaccacgcgaggcgcgagataggggggca
cgggcgcgaccatctgcgctgcggcgccggcgactcagcgctgcctcagtctgcggtgggcagc
ggaggagtcgtgtcgtgcctgagagcgcagtcgagaaggtaccGGATCCGCCACCatgggcaag
cccatccccaaccccctgctgggcctggacagcaccGGCGGTGGAGGTTCCtccaaaaccatcg
ttctttcggtcggcgaggctactcgcactctgactgagatccagtccaccgcagaccgtcagat
cttcgaagagaaggtcgggcctctggtgggtcggctgcgcctcacggcttcgctccgtcaaaac
ggagccaagaccgcgtatcgcgtcaacctaaaactggatcaggcggacgtcgttgattgctcca
ccagcgtctgcggcgagcttccgaaagtgcgctacactcaggtatggtcgcacgacgtgacaat
cgttgcgaatagcaccgaggcctcgcgcaaatcgttgtacgatttgaccaagtccctcgtcgcg
acctcgcaggtcgaagatcttgtcgtcaaccttgtgccgctgggccgtggcggtggcggatctg
gcggcggtggtagcAATGATTTTGGCAATTACAACAATCAGTCTTCCAATTTTGGGCCGATGAA
GGGAGGAAACTTTGGAGGCAGGAGCTCTGGCCCTTATGGTGGTGGAGGCCAGTACTTTGCTAAA
CCACGGAACCAAGGTGGCTATGGCGGAGGTGGTTCTctgcctccacttgaaagactgacactgg
gctcaggaggatctggtggttctgagggcagaggaagtcttctaacatgcggtgacgtggagga
gaatcccggccctgCCatggattacaaggatgacgacgataagGGCGGAGGTGGTTCTtccaaa
accatcgttctttcggtcggcgaggctactcgcactctgactgagatccagtccaccgcagacc
gtcagatcttcgaagagaaggtcgggcctctggtgggtcggctgcgcctcacggcttcgctccg
tcaaaacggagccaagaccgcgtatcgcgtcaacctaaaactggatcaggcggacgtcgttgat
tgctccaccagcgtctgcggcgagcttccgaaagtgcgctacactcaggtatggtcgcacgacg
tgacaatcgttgcgaatagcaccgaggcctcgcgcaaatcgttgtacgatttgaccaagtccct
cgtcgcgacctcgcaggtcgaagatcttgtcgtcaaccttgtgccgctgggccgtGGCGGAGGT
GGTTCTaagctgaaccctcctgatgagagtggccccggctgcatgagctgctgtgtgctctcct
aagaattcgatatcaagcttatcgataatcaacctctggattacaaaatttgtgaaagattgac
tggtattcttaactatgttgctccttttacgctatgtggatacgctgctttaatgcctttgtat
catgctattgcttcccgtatggctttcattttctcctccttgtataaatcctggttgctgtctc
tttatgaggagttgtggcccgttgtcaggcaacgtggcgtggtgtgcactgtgtttgctgacgc
aacccccactggttggggcattgccaccacctgtcagctcctttccgggactttcgctttcccc
ctccctattgccacggcggaactcatcgccgcctgccttgcccgctgctggacaggggctcggc
tgttgggcactgacaattccgtggtgttgtcggggaaatcatcgtcctttccttggctgctcgc
ctatgttgccacctggattctgcgcgggacgtccttctgctacgtcccttcggccctcaatcca
gcggaccttccttcccgcggcctgctgccggctctgcggcctcttccgcgtcttcgccttcgcc
ctcagacgagtcggatctccctttgggccgcctccccgcatcgataccgagcgctgctcgagag
atctacgggtggcatccctgtgacccctccccagtgcctctcctggccctggaagttgccactc
cagtgcccaccagccttgtcctaataaaattaagttgcatcattttgtctgactaggtgtcctt
ctataatattatggggtggaggggggtggtatggagcaaggggcaagttgggaagacaacctgt
agggcctgcggggtctattgggaaccaagctggagtgcagtggcacaatcttggctcactgcaa
tctccgcctcctgggttcaagcgattctcctgcctcagcctcccgagttgttgggattccaggc
atgcatgaccaggctcagctaatttttgtttttttggtagagacggggtttcaccatattggcc
aggctggtctccaactcctaatctcaggtgatctacccaccttggcctcccaaattgctgggat
tacaggcgtgaaccactgctcccttccctgtccttctgattttgtaggtaaccacgtgcggacc
gagcggccgcaggaacccctagtgatggagttggccactccctctctgcgcgctcgctcgctca
ctgaggccgggcgaccaaaggtcgcccgacgcccgggctttgcccgggcggcctcagtgagcga
gcgagcgcgcagctgcctgcaggggcgcctgatgcggtattttctccttacgcatctgtgcggt
atttcacaccgcatacgtcaaagcaaccatagtacgcgccctgtagcggcgcattaagcgcggc
gggtgtggtggttacgcgcagcgtgaccgctacacttgccagcgccttagcgcccgctcctttc
gctttcttcccttcctttctcgccacgttcgccggctttccccgtcaagctctaaatcgggggc
tccctttagggttccgatttagtgctttacggcacctcgaccccaaaaaacttgatttgggtga
tggttcacgtagtgggccatcgccctgatagacggtttttcgccctttgacgttggagtccacg
ttctttaatagtggactcttgttccaaactggaacaacactcaactctatctcgggctattctt
ttgatttataagggattttgccgatttcggtctattggttaaaaaatgagctgatttaacaaaa
atttaacgcgaattttaacaaaatattaacgtttacaattttatggtgcactctcagtacaatc
tgctctgatgccgcatagttaagccagccccgacacccgccaacacccgctgacgcgccctgac
gggcttgtctgctcccggcatccgcttacagacaagctgtgaccgtctccgggagctgcatgtg
tcagaggttttcaccgtcatcaccgaaacgcgcgagacgaaagggcctcgtgatacgcctattt
ttataggttaatgtcatgataataatggtttcttagacgtcaggtggcacttttcggggaaatg
tgcgcggaacccctatttgtttatttttctaaatacattcaaatatgtatccgctcatgagaca
ataaccctgataaatgcttcaataatattgaaaaaggaagagtatgagtattcaacatttccgt
gtcgcccttattcccttttttgcggcattttgccttcctgtttttgctcacccagaaacgctgg
tgaaagtaaaagatgctgaagatcagttgggtgcacgagtgggttacatcgaactggatctcaa
cagcggtaagatccttgagagttttcgccccgaagaacgttttccaatgatgagcacttttaaa
gttctgctatgtggcgcggtattatcccgtattgacgccgggcaagagcaactcggtcgccgca
tacactattctcagaatgacttggttgagtactcaccagtcacagaaaagcatcttacggatgg
catgacagtaagagaattatgcagtgctgccataaccatgagtgataacactgcggccaactta
cttctgacaacgatcggaggaccgaaggagctaaccgcttttttgcacaacatgggggatcatg
taactcgccttgatcgttgggaaccggagctgaatgaagccataccaaacgacgagcgtgacac
cacgatgcctgtagcaatggcaacaacgttgcgcaaactattaactggcgaactacttactcta
gcttcccggcaacaattaatagactggatggaggcggataaagttgcaggaccacttctgcgct
cggcccttccggctggctggtttattgctgataaatctggagccggtgagcgtgggtctcgcgg
tatcattgcagcactggggccagatggtaagccctcccgtatcgtagttatctacacgacgggg
agtcaggcaactatggatgaacgaaatagacagatcgctgagataggtgcctcactgattaagc
attggtaactgtcagaccaagtttactcatatatactttagattgatttaaaacttcattttta
atttaaaaggatctaggtgaagatcctttttgataatctcatgaccaaaatcccttaacgtgag
ttttcgttccactgagcgtcagaccccgtagaaaagatcaaaggatcttcttgagatccttttt
ttctgcgcgtaatctgctgcttgcaaacaaaaaaaccaccgctaccagcggtggtttgtttgcc
ggatcaagagctaccaactctttttccgaaggtaactggcttcagcagagcgcagataccaaat
actgttcttctagtgtagccgtagttaggccaccacttcaagaactctgtagcaccgcctacat
acctcgctctgctaatcctgttaccagtggctgctgccagtggcgataagtcgtgtcttaccgg
gttggactcaagacgatagttaccggataaggcgcagcggtcgggctgaacggggggttcgtgc
acacagcccagcttggagcgaacgacctacaccgaactgagatacctacagcgtgagctatgag
aaagcgccacgcttcccgaagggagaaaggcggacaggtatccggtaagcggcagggtcggaac
aggagagcgcacgagggagcttccagggggaaacgcctggtatctttatagtcctgtcgggttt
cgccacctctgacttgagcgtcgatttttgtgatgctcgtcaggggggcggagcctatggaaaa
acgccagcaacgcggcctttttacggttcctggccttttgctggccttttgctcacatgt

Plasmid #18 (see FIG. 38 for a map of the plasmid)

(SEQ ID NO: 87)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatCCTAGGT
TAAGGATGCACCGACGGGACGTTCTATGAGGACGAATCTCCCGCTTATAAGATTCTATAAACTG
TGCGGTCCTTCAATTGgggtcccCACTAACCTAAGACAGGAGGGCCGGGAAACCTGCCTAATCC
AATGACGGGTAATAGTGgggacccatcattcatggcaagtggccgcggtcggcgtggactgtag
aacactgccaatgccggtcccaagcccggataaaaGTGGAGGGTACAGTCCACGCtttttttct
agactgcagagggccctgcgtatgagtgcaagtgggttttaggaccaggatgaggcggggtggg
ggtgcctacctgacgaccgaccccgacccactggacaagcacccaacccccattccccaaattg
cgcatcccctatcagagagggggaggggaaacaggatgcggcgaggcgcgtgcgcactgccagc
ttcagcaccgcggacagtgccttcgcccccgcctggcggcgcgcgccaccgccgcctcagcact
gaaggcgcgctgacgtcactcgccggtcccccgcaaactccccttcccggccaccttggtcgcg
tccgcgccgccgccggcccagccggaccgcaccacgcgaggcgcgagataggggggcacgggcg
cgaccatctgcgctgcggcgccggcgactcagcgctgcctcagtctgcggtgggcagcggagga
gtcgtgtcgtgcctgagagcgcagtcgagaaggtaccGGATCCGCCACCatgggcaagcccatc
cccaaccccctgctgggcctggacagcaccGGCGGTGGAGGTTCCtccaaaaccatcgttcttt
cggtcggcgaggctactcgcactctgactgagatccagtccaccgcagaccgtcagatcttcga
agagaaggtcgggcctctggtgggtcggctgcgcctcacggcttcgctccgtcaaaacggagcc
aagaccgcgtatcgcgtcaacctaaaactggatcaggcggacgtcgttgattgctccaccagcg
tctgcggcgagcttccgaaagtgcgctacactcaggtatggtcgcacgacgtgacaatcgttgc
gaatagcaccgaggcctcgcgcaaatcgttgtacgatttgaccaagtccctcgtcgcgacctcg
caggtcgaagatcttgtcgtcaaccttgtgccgctgggccgtggcggtggcggatctggcggcg
gtggtagcAATGATTTTGGCAATTACAACAATCAGTCTTCCAATTTTGGGCCGATGAAGGGAGG
AAACTTTGGAGGCAGGAGCTCTGGCCCTTATGGTGGTGGAGGCCAGTACTTTGCTAAACCACGG
AACCAAGGTGGCTATGGCGGAGGTGGTTCTctgcctccacttgaaagactgacactgggctcag
gaggatctggtggttctgagggcagaggaagtcttctaacatgcggtgacgtggaggagaatcc
cggccctgCCatggattacaaggatgacgacgataagGGCGGAGGTGGTTCTtccaaaaccatc
gttctttcggtcggcgaggctactcgcactctgactgagatccagtccaccgcagaccgtcaga
tcttcgaagagaaggtcgggcctctggtgggtcggctgcgcctcacggcttcgctccgtcaaaa
cggagccaagaccgcgtatcgcgtcaacctaaaactggatcaggcggacgtcgttgattgctcc
accagcgtctgcggcgagcttccgaaagtgcgctacactcaggtatggtcgcacgacgtgacaa
tcgttgcgaatagcaccgaggcctcgcgcaaatcgttgtacgatttgaccaagtccctcgtcgc
gacctcgcaggtcgaagatcttgtcgtcaaccttgtgccgctgggccgtGGCGGAGGTGGTTCT
aagctgaaccctcctgatgagagtggccccggctgcatgagctgctgtgtgctctcctaagaat
tcgatatcaagcttatcgataatcaacctctggattacaaaatttgtgaaagattgactggtat
tcttaactatgttgctccttttacgctatgtggatacgctgctttaatgcctttgtatcatgct
attgcttcccgtatggctttcattttctcctccttgtataaatcctggttgctgtctctttatg
aggagttgtggcccgttgtcaggcaacgtggcgtggtgtgcactgtgtttgctgacgcaacccc
cactggttggggcattgccaccacctgtcagctcctttccgggactttcgctttccccctccct
attgccacggcggaactcatcgccgcctgccttgcccgctgctggacaggggctcggctgttgg
gcactgacaattccgtggtgttgtcggggaaatcatcgtcctttccttggctgctcgcctatgt
tgccacctggattctgcgcgggacgtccttctgctacgtcccttcggccctcaatccagcggac
cttccttcccgcggcctgctgccggctctgcggcctcttccgcgtcttcgccttcgccctcaga
cgagtcggatctccctttgggccgcctccccgcatcgataccgagcgctgctcgagagatctac
gggtggcatccctgtgacccctccccagtgcctctcctggccctggaagttgccactccagtgc
ccaccagccttgtcctaataaaattaagttgcatcattttgtctgactaggtgtccttctataa
tattatggggtggaggggggtggtatggagcaaggggcaagttgggaagacaacctgtagggcc
tgcggggtctattgggaaccaagctggagtgcagtggcacaatcttggctcactgcaatctccg
cctcctgggttcaagcgattctcctgcctcagcctcccgagttgttgggattccaggcatgcat
gaccaggctcagctaatttttgtttttttggtagagacggggtttcaccatattggccaggctg
gtctccaactcctaatctcaggtgatctacccaccttggcctcccaaattgctgggattacagg
cgtgaaccactgctcccttccctgtccttctgattttgtaggtaaccacgtgcggaccgagcgg
ccgcaggaacccctagtgatggagttggccactccctctctgcgcgctcgctcgctcactgagg
ccgggcgaccaaaggtcgcccgacgcccgggctttgcccgggcggcctcagtgagcgagcgagc
gcgcagctgcctgcaggggcgcctgatgcggtattttctccttacgcatctgtgcggtatttca
caccgcatacgtcaaagcaaccatagtacgcgccctgtagcggcgcattaagcgcggcgggtgt
ggtggttacgcgcagcgtgaccgctacacttgccagcgccttagcgcccgctcctttcgctttc
ttcccttcctttctcgccacgttcgccggctttccccgtcaagctctaaatcgggggctccctt
tagggttccgatttagtgctttacggcacctcgaccccaaaaaacttgatttgggtgatggttc
acgtagtgggccatcgccctgatagacggtttttcgccctttgacgttggagtccacgttcttt
aatagtggactcttgttccaaactggaacaacactcaactctatctcgggctattcttttgatt
tataagggattttgccgatttcggtctattggttaaaaaatgagctgatttaacaaaaatttaa
cgcgaattttaacaaaatattaacgtttacaattttatggtgcactctcagtacaatctgctct
gatgccgcatagttaagccagccccgacacccgccaacacccgctgacgcgccctgacgggctt
gtctgctcccggcatccgcttacagacaagctgtgaccgtctccgggagctgcatgtgtcagag
gttttcaccgtcatcaccgaaacgcgcgagacgaaagggcctcgtgatacgcctatttttatag
gttaatgtcatgataataatggtttcttagacgtcaggtggcacttttcggggaaatgtgcgcg
gaacccctatttgtttatttttctaaatacattcaaatatgtatccgctcatgagacaataacc
ctgataaatgcttcaataatattgaaaaaggaagagtatgagtattcaacatttccgtgtcgcc
cttattcccttttttgcggcattttgccttcctgtttttgctcacccagaaacgctggtgaaag
taaaagatgctgaagatcagttgggtgcacgagtgggttacatcgaactggatctcaacagcgg
taagatccttgagagttttcgccccgaagaacgttttccaatgatgagcacttttaaagttctg
ctatgtggcgcggtattatcccgtattgacgccgggcaagagcaactcggtcgccgcatacact
attctcagaatgacttggttgagtactcaccagtcacagaaaagcatcttacggatggcatgac
agtaagagaattatgcagtgctgccataaccatgagtgataacactgcggccaacttacttctg
acaacgatcggaggaccgaaggagctaaccgcttttttgcacaacatgggggatcatgtaactc
gccttgatcgttgggaaccggagctgaatgaagccataccaaacgacgagcgtgacaccacgat
gcctgtagcaatggcaacaacgttgcgcaaactattaactggcgaactacttactctagcttcc
cggcaacaattaatagactggatggaggcggataaagttgcaggaccacttctgcgctcggccc
ttccggctggctggtttattgctgataaatctggagccggtgagcgtgggtctcgcggtatcat
tgcagcactggggccagatggtaagccctcccgtatcgtagttatctacacgacggggagtcag
gcaactatggatgaacgaaatagacagatcgctgagataggtgcctcactgattaagcattggt
aactgtcagaccaagtttactcatatatactttagattgatttaaaacttcatttttaatttaa
aaggatctaggtgaagatcctttttgataatctcatgaccaaaatcccttaacgtgagttttcg
ttccactgagcgtcagaccccgtagaaaagatcaaaggatcttcttgagatcctttttttctgc
gcgtaatctgctgcttgcaaacaaaaaaaccaccgctaccagcggtggtttgtttgccggatca
agagctaccaactctttttccgaaggtaactggcttcagcagagcgcagataccaaatactgtt
cttctagtgtagccgtagttaggccaccacttcaagaactctgtagcaccgcctacatacctcg
ctctgctaatcctgttaccagtggctgctgccagtggcgataagtcgtgtcttaccgggttgga
ctcaagacgatagttaccggataaggcgcagcggtcgggctgaacggggggttcgtgcacacag
cccagcttggagcgaacgacctacaccgaactgagatacctacagcgtgagctatgagaaagcg
ccacgcttcccgaagggagaaaggcggacaggtatccggtaagcggcagggtcggaacaggaga
gcgcacgagggagcttccagggggaaacgcctggtatctttatagtcctgtcgggtttcgccac
ctctgacttgagcgtcgatttttgtgatgctcgtcaggggggcggagcctatggaaaaacgcca
gcaacgcggcctttttacggttcctggccttttgctggccttttgctcacatgt

Plasmid #19 (see FIG. 39 for a map of the plasmid)

(SEQ ID NO: 112)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggggggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggcccTAATGATTAACCCGCCATGCTACTTATCTACGTAGCCATGCT
CTAGGAAGATCGTACCATTGACGTCAATAATGACGTATGTTCCCATAGTAACGCCAATAGGGAC
TTTCCATTGACGTCAATGGGTGGACTATTTACGGTAAACTGCCCACTTGGCAGTACATCAAGTG
TATCATATGCCAAGTACGCCCCCTATTGACGTCAATGACGGTAAATGGCCCGCCTGGCATTATG
CCCAGTACATGACCTTATGGGACTTTCCTACTTGGCAGTACATCTACGTATTAGTCATCGCTAT
TACCATGGTCGAGGTGAGCCCCACGTTCTGCTTCACTCTCCCCATCTCCCCCCCCTCCCCACCC
CCAATTTTGTATTTATTTATTTTTTAATTATTTTGTGCAGCGATGGGGGGGGGGGGGGGGGGGG
GGCGCGCGCCAGGCGGGGCGGGGGCGGGGCGAGGGGCGGGGGGGGCGAGGCGGAGAGGTGCGGC
GGCAGCCAATCAGAGCGGCGCGCTCCGAAAGTTTCCTTTTATGGCGAGGCGGCGGCGGCGGCGG
CCCTATAAAAAGCGAAGCGCGCGGCGGGGGGGAGTCGCTGCGACGCTGCCTTCGCCCCGTGCCC
CGCTCCGCCGCCGCCTCGCGCCGCCCGCCCCGGCTCTGACTGACCGCGTTACTCCCACAGGTGA
GCGGGCGGGACGGCCCTTCTCCTCCGGGCTGTAATTAGCGCTTGGTTTAATGACGGCTTGTTTC
TTTTCTGTGGCTGCGTGAAAGCCTTGAGGGGCTCCGGGAGGGCCCTTTGTGCGGGGGGAGCGGC
TCGGGGCTGTCCGCGGGGGGACGGCTGCCTTCGGGGGGGACGGGGCAGGGGGGGGTTCGGCTTC
TGGCGTGTGACCGGCGGCTCTAGAGCCTCTGCTAACCATGTTCATGCCTTCTTCTTTTTCCTAC
AGCTCCTGGGCAACGTGCTGGTTATTGTGCTGTCTCATCATTTTGGCAAAGAATTGGATCCGCC
ACCatggattacaaggatgacgacgataagGGCGGAGGTGGTTCTtccaaaaccatcgttcttt
cggtcggcgaggctactcgcactctgactgagatccagtccaccgcagaccgtcagatcttcga
agagaaggtcgggcctctggtgggtcggctgcgcctcacggcttcgctccgtcaaaacggagcc
aagaccgcgtatcgcgtcaacctaaaactggatcaggcggacgtcgttgattgctccaccagcg
tctgcggcgagcttccgaaagtgcgctacactcaggtatggtcgcacgacgtgacaatcgttgc
gaatagcaccgaggcctcgcgcaaatcgttgtacgatttgaccaagtccctcgtcgcgacctcg
caggtcgaagatcttgtcgtcaaccttgtgccgctgggccgtGGCGGAGGTGGTTCTaagctga
accctcctgatgagagtggccccggctgcatgagctgctgtgtgctctcctaagaattcgatat
caagcttatcgataatcaacctctggattacaaaatttgtgaaagattgactggtattcttaac
tatgttgctccttttacgctatgtggatacgctgctttaatgcctttgtatcatgctattgctt
cccgtatggctttcattttctcctccttgtataaatcctggttgctgtctctttatgaggagtt
gtggcccgttgtcaggcaacgtggcgtggtgtgcactgtgtttgctgacgcaacccccactggt
tggggcattgccaccacctgtcagctcctttccgggactttcgctttccccctccctattgcca
cggcggaactcatcgccgcctgccttgcccgctgctggacaggggctcggctgttgggcactga
caattccgtggtgttgtcggggaaatcatcgtcctttccttggctgctcgcctatgttgccacc
tggattctgcgcgggacgtccttctgctacgtcccttcggccctcaatccagcggaccttcctt
cccgcggcctgctgccggctctgcggcctcttccgcgtcttcgccttcgccctcagacgagtcg
gatctccctttgggccgcctccccgcatcgataccgagcgctgctcgagagatctacgggtggc
atccctgtgacccctccccagtgcctctcctggccctggaagttgccactccagtgcccaccag
ccttgtcctaataaaattaagttgcatcattttgtctgactaggtgtccttctataatattatg
gggtggaggggggtggtatggagcaaggggcaagttgggaagacaacctgtagggcctgcgggg
tctattgggaaccaagctggagtgcagtggcacaatcttggctcactgcaatctccgcctcctg
ggttcaagcgattctcctgcctcagcctcccgagttgttgggattccaggcatgcatgaccagg
ctcagctaatttttgtttttttggtagagacggggtttcaccatattggccaggctggtctcca
actcctaatctcaggtgatctacccaccttggcctcccaaattgctgggattacaggcgtgaac
cactgctcccttccctgtccttctgattttgtaggtaaccacgtgcggaccgagcggccgcagg
aacccctagtgatggagttggccactccctctctgcgcgctcgctcgctcactgaggccgggcg
accaaaggtcgcccgacgcccgggctttgcccgggcggcctcagtgagcgagcgagcgcgcagc
tgcctgcaggggcgcctgatgcggtattttctccttacgcatctgtgcggtatttcacaccgca
tacgtcaaagcaaccatagtacgcgccctgtagcggcgcattaagcgcggcgggtgtggtggtt
acgcgcagcgtgaccgctacacttgccagcgccttagcgcccgctcctttcgctttcttccctt
cctttctcgccacgttcgccggctttccccgtcaagctctaaatcgggggctccctttagggtt
ccgatttagtgctttacggcacctcgaccccaaaaaacttgatttgggtgatggttcacgtagt
gggccatcgccctgatagacggtttttcgccctttgacgttggagtccacgttctttaatagtg
gactcttgttccaaactggaacaacactcaactctatctcgggctattcttttgatttataagg
gattttgccgatttcggtctattggttaaaaaatgagctgatttaacaaaaatttaacgcgaat
tttaacaaaatattaacgtttacaattttatggtgcactctcagtacaatctgctctgatgccg
catagttaagccagccccgacacccgccaacacccgctgacgcgccctgacgggcttgtctgct
cccggcatccgcttacagacaagctgtgaccgtctccgggagctgcatgtgtcagaggttttca
ccgtcatcaccgaaacgcgcgagacgaaagggcctcgtgatacgcctatttttataggttaatg
tcatgataataatggtttcttagacgtcaggtggcacttttcggggaaatgtgcgcggaacccc
tatttgtttatttttctaaatacattcaaatatgtatccgctcatgagacaataaccctgataa
atgcttcaataatattgaaaaaggaagagtatgagtattcaacatttccgtgtcgcccttattc
ccttttttgcggcattttgccttcctgtttttgctcacccagaaacgctggtgaaagtaaaaga
tgctgaagatcagttgggtgcacgagtgggttacatcgaactggatctcaacagcggtaagatc
cttgagagttttcgccccgaagaacgttttccaatgatgagcacttttaaagttctgctatgtg
gcgcggtattatcccgtattgacgccgggcaagagcaactcggtcgccgcatacactattctca
gaatgacttggttgagtactcaccagtcacagaaaagcatcttacggatggcatgacagtaaga
gaattatgcagtgctgccataaccatgagtgataacactgcggccaacttacttctgacaacga
tcggaggaccgaaggagctaaccgcttttttgcacaacatgggggatcatgtaactcgccttga
tcgttgggaaccggagctgaatgaagccataccaaacgacgagcgtgacaccacgatgcctgta
gcaatggcaacaacgttgcgcaaactattaactggcgaactacttactctagcttcccggcaac
aattaatagactggatggaggcggataaagttgcaggaccacttctgcgctcggcccttccggc
tggctggtttattgctgataaatctggagccggtgagcgtgggtctcgcggtatcattgcagca
ctggggccagatggtaagccctcccgtatcgtagttatctacacgacggggagtcaggcaacta
tggatgaacgaaatagacagatcgctgagataggtgcctcactgattaagcattggtaactgtc
agaccaagtttactcatatatactttagattgatttaaaacttcatttttaatttaaaaggatc
taggtgaagatcctttttgataatctcatgaccaaaatcccttaacgtgagttttcgttccact
gagcgtcagaccccgtagaaaagatcaaaggatcttcttgagatcctttttttctgcgcgtaat
ctgctgcttgcaaacaaaaaaaccaccgctaccagcggtggtttgtttgccggatcaagagcta
ccaactctttttccgaaggtaactggcttcagcagagcgcagataccaaatactgttcttctag
tgtagccgtagttaggccaccacttcaagaactctgtagcaccgcctacatacctcgctctgct
aatcctgttaccagtggctgctgccagtggcgataagtcgtgtcttaccgggttggactcaaga
cgatagttaccggataaggcgcagcggtcgggctgaacggggggttcgtgcacacagcccagct
tggagcgaacgacctacaccgaactgagatacctacagcgtgagctatgagaaagcgccacgct
tcccgaagggagaaaggcggacaggtatccggtaagcggcagggtcggaacaggagagcgcacg
agggagcttccagggggaaacgcctggtatctttatagtcctgtcgggtttcgccacctctgac
ttgagcgtcgatttttgtgatgctcgtcaggggggcggagcctatggaaaaacgccagcaacgc
ggcctttttacggttcctggccttttgctggccttttgctcacatgt

Plasmid #20 (see FIG. 40 for a map of the plasmid)

(SEQ ID NO: 113)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGgggtcccatcattcatggcaagtggccgcggtcggcgtgga
ctgtagaacactgccaatgccggtcccaagcccggataaaaGTGGAGGGTACAGTCCACGCttt
ttttctagactgcagagggcccTAATGATTAACCCGCCATGCTACTTATCTACGTAGCCATGCT
CTAGGAAGATCGTACCATTGACGTCAATAATGACGTATGTTCCCATAGTAACGCCAATAGGGAC
TTTCCATTGACGTCAATGGGTGGACTATTTACGGTAAACTGCCCACTTGGCAGTACATCAAGTG
TATCATATGCCAAGTACGCCCCCTATTGACGTCAATGACGGTAAATGGCCCGCCTGGCATTATG
CCCAGTACATGACCTTATGGGACTTTCCTACTTGGCAGTACATCTACGTATTAGTCATCGCTAT
TACCATGGTCGAGGTGAGCCCCACGTTCTGCTTCACTCTCCCCATCTCCCCCCCCTCCCCACCC
CCAATTTTGTATTTATTTATTTTTTAATTATTTTGTGCAGCGATGGGGGGGGGGGGGGGGGGGG
GGCGCGCGCCAGGCGGGGGGGGGCGGGGCGAGGGGGGGGGGGGGGCGAGGCGGAGAGGTGCGGC
GGCAGCCAATCAGAGCGGCGCGCTCCGAAAGTTTCCTTTTATGGCGAGGCGGCGGCGGCGGCGG
CCCTATAAAAAGCGAAGCGCGCGGCGGGCGGGAGTCGCTGCGACGCTGCCTTCGCCCCGTGCCC
CGCTCCGCCGCCGCCTCGCGCCGCCCGCCCCGGCTCTGACTGACCGCGTTACTCCCACAGGTGA
GCGGGCGGGACGGCCCTTCTCCTCCGGGCTGTAATTAGCGCTTGGTTTAATGACGGCTTGTTTC
TTTTCTGTGGCTGCGTGAAAGCCTTGAGGGGCTCCGGGAGGGCCCTTTGTGCGGGGGGAGCGGC
TCGGGGCTGTCCGCGGGGGGACGGCTGCCTTCGGGGGGGACGGGGCAGGGCGGGGTTCGGCTTC
TGGCGTGTGACCGGCGGCTCTAGAGCCTCTGCTAACCATGTTCATGCCTTCTTCTTTTTCCTAC
AGCTCCTGGGCAACGTGCTGGTTATTGTGCTGTCTCATCATTTTGGCAAAGAATTGGATCCGCC
ACCatgggcaagcccatccccaaccccctgctgggcctggacagcaccGGCGGTGGAGGTTCCt
ccaaaaccatcgttctttcggtcggcgaggctactcgcactctgactgagatccagtccaccgc
agaccgtcagatcttcgaagagaaggtcgggcctctggtgggtcggctgcgcctcacggcttcg
ctccgtcaaaacggagccaagaccgcgtatcgcgtcaacctaaaactggatcaggcggacgtcg
ttgattgctccaccagcgtctgcggcgagcttccgaaagtgcgctacactcaggtatggtcgca
cgacgtgacaatcgttgcgaatagcaccgaggcctcgcgcaaatcgttgtacgatttgaccaag
tccctcgtcgcgacctcgcaggtcgaagatcttgtcgtcaaccttgtgccgctgggccgtggcg
gtggcggatctggcggcggtggtagcAATGATTTTGGCAATTACAACAATCAGTCTTCCAATTT
TGGGCCGATGAAGGGAGGAAACTTTGGAGGCAGGAGCTCTGGCCCTTATGGTGGTGGAGGCCAG
TACTTTGCTAAACCACGGAACCAAGGTGGCTATGGCGGAGGTGGTTCTctgcctccacttgaaa
gactgacactgggctcaggaggatctggtggttctgagggcagaggaagtcttctaacatgcgg
tgacgtggaggagaatcccggccctgCCatggattacaaggatgacgacgataagGGCGGAGGT
GGTTCTtccaaaaccatcgttctttcggtcggcgaggctactcgcactctgactgagatccagt
ccaccgcagaccgtcagatcttcgaagagaaggtcgggcctctggtgggtcggctgcgcctcac
ggcttcgctccgtcaaaacggagccaagaccgcgtatcgcgtcaacctaaaactggatcaggcg
gacgtcgttgattgctccaccagcgtctgcggcgagcttccgaaagtgcgctacactcaggtat
ggtcgcacgacgtgacaatcgttgcgaatagcaccgaggcctcgcgcaaatcgttgtacgattt
gaccaagtccctcgtcgcgacctcgcaggtcgaagatcttgtcgtcaaccttgtgccgctgggc
cgtGGCGGAGGTGGTTCTaagctgaaccctcctgatgagagtggccccggctgcatgagctgct
gtgtgctctcctaagaattcgatatcaagcttatcgataatcaacctctggattacaaaatttg
tgaaagattgactggtattcttaactatgttgctccttttacgctatgtggatacgctgcttta
atgcctttgtatcatgctattgcttcccgtatggctttcattttctcctccttgtataaatcct
ggttgctgtctctttatgaggagttgtggcccgttgtcaggcaacgtggcgtggtgtgcactgt
gtttgctgacgcaacccccactggttggggcattgccaccacctgtcagctcctttccgggact
ttcgctttccccctccctattgccacggcggaactcatcgccgcctgccttgcccgctgctgga
caggggctcggctgttgggcactgacaattccgtggtgttgtcggggaaatcatcgtcctttcc
ttggctgctcgcctatgttgccacctggattctgcgcgggacgtccttctgctacgtcccttcg
gccctcaatccagcggaccttccttcccgcggcctgctgccggctctgcggcctcttccgcgtc
ttcgccttcgccctcagacgagtcggatctccctttgggccgcctccccgcatcgataccgagc
gctgctcgagagatctacgggtggcatccctgtgacccctccccagtgcctctcctggccctgg
aagttgccactccagtgcccaccagccttgtcctaataaaattaagttgcatcattttgtctga
ctaggtgtccttctataatattatggggtggaggggggtggtatggagcaaggggcaagttggg
aagacaacctgtagggcctgcggggtctattgggaaccaagctggagtgcagtggcacaatctt
ggctcactgcaatctccgcctcctgggttcaagcgattctcctgcctcagcctcccgagttgtt
gggattccaggcatgcatgaccaggctcagctaatttttgtttttttggtagagacggggtttc
accatattggccaggctggtctccaactcctaatctcaggtgatctacccaccttggcctccca
aattgctgggattacaggcgtgaaccactgctcccttccctgtccttctgattttgtaggtaac
cacgtgcggaccgagcggccgcaggaacccctagtgatggagttggccactccctctctgcgcg
ctcgctcgctcactgaggccgggcgaccaaaggtcgcccgacgcccgggctttgcccgggcggc
ctcagtgagcgagcgagcgcgcagctgcctgcaggggcgcctgatgcggtattttctccttacg
catctgtgcggtatttcacaccgcatacgtcaaagcaaccatagtacgcgccctgtagcggcgc
attaagcgcggcgggtgtggtggttacgcgcagcgtgaccgctacacttgccagcgccttagcg
cccgctcctttcgctttcttcccttcctttctcgccacgttcgccggctttccccgtcaagctc
taaatcgggggctccctttagggttccgatttagtgctttacggcacctcgaccccaaaaaact
tgatttgggtgatggttcacgtagtgggccatcgccctgatagacggtttttcgccctttgacg
ttggagtccacgttctttaatagtggactcttgttccaaactggaacaacactcaactctatct
cgggctattcttttgatttataagggattttgccgatttcggtctattggttaaaaaatgagct
gatttaacaaaaatttaacgcgaattttaacaaaatattaacgtttacaattttatggtgcact
ctcagtacaatctgctctgatgccgcatagttaagccagccccgacacccgccaacacccgctg
acgcgccctgacgggcttgtctgctcccggcatccgcttacagacaagctgtgaccgtctccgg
gagctgcatgtgtcagaggttttcaccgtcatcaccgaaacgcgcgagacgaaagggcctcgtg
atacgcctatttttataggttaatgtcatgataataatggtttcttagacgtcaggtggcactt
ttcggggaaatgtgcgcggaacccctatttgtttatttttctaaatacattcaaatatgtatcc
gctcatgagacaataaccctgataaatgcttcaataatattgaaaaaggaagagtatgagtatt
caacatttccgtgtcgcccttattcccttttttgcggcattttgccttcctgtttttgctcacc
cagaaacgctggtgaaagtaaaagatgctgaagatcagttgggtgcacgagtgggttacatcga
actggatctcaacagcggtaagatccttgagagttttcgccccgaagaacgttttccaatgatg
agcacttttaaagttctgctatgtggcgcggtattatcccgtattgacgccgggcaagagcaac
tcggtcgccgcatacactattctcagaatgacttggttgagtactcaccagtcacagaaaagca
tcttacggatggcatgacagtaagagaattatgcagtgctgccataaccatgagtgataacact
gcggccaacttacttctgacaacgatcggaggaccgaaggagctaaccgcttttttgcacaaca
tgggggatcatgtaactcgccttgatcgttgggaaccggagctgaatgaagccataccaaacga
cgagcgtgacaccacgatgcctgtagcaatggcaacaacgttgcgcaaactattaactggcgaa
ctacttactctagcttcccggcaacaattaatagactggatggaggcggataaagttgcaggac
cacttctgcgctcggcccttccggctggctggtttattgctgataaatctggagccggtgagcg
tgggtctcgcggtatcattgcagcactggggccagatggtaagccctcccgtatcgtagttatc
tacacgacggggagtcaggcaactatggatgaacgaaatagacagatcgctgagataggtgcct
cactgattaagcattggtaactgtcagaccaagtttactcatatatactttagattgatttaaa
acttcatttttaatttaaaaggatctaggtgaagatcctttttgataatctcatgaccaaaatc
ccttaacgtgagttttcgttccactgagcgtcagaccccgtagaaaagatcaaaggatcttctt
gagatcctttttttctgcgcgtaatctgctgcttgcaaacaaaaaaaccaccgctaccagcggt
ggtttgtttgccggatcaagagctaccaactctttttccgaaggtaactggcttcagcagagcg
cagataccaaatactgttcttctagtgtagccgtagttaggccaccacttcaagaactctgtag
caccgcctacatacctcgctctgctaatcctgttaccagtggctgctgccagtggcgataagtc
gtgtcttaccgggttggactcaagacgatagttaccggataaggcgcagcggtcgggctgaacg
gggggttcgtgcacacagcccagcttggagcgaacgacctacaccgaactgagatacctacagc
gtgagctatgagaaagcgccacgcttcccgaagggagaaaggcggacaggtatccggtaagcgg
cagggtcggaacaggagagcgcacgagggagcttccagggggaaacgcctggtatctttatagt
cctgtcgggtttcgccacctctgacttgagcgtcgatttttgtgatgctcgtcaggggggcgga
gcctatggaaaaacgccagcaacgcggcctttttacggttcctggccttttgctggccttttgc
tcacatgt

Plasmid #21 (see FIG. 41 for a map of the plasmid)

(SEQ ID NO: 114)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatCCTAGGG
ACGGGACGTTCTATGAGGACGAATCTCCCGCTTATAAGATTCTATAAACTGTGCGGTCCTTCAA
TTGgggtcccCACTAACCTAAGACAGGAGGGCCGGGAAACCTGCCTAATCCAATGACGGGTAAT
AGTGgggacccatcattcatggcaagtggccgcggtcggcgtggactgtagaacactgccaatg
ccggtcccaagcccggataaaaGTGGAGGGTACAGTCCACGCtttttttctagactgcagaggg
ccctaatgattaacccgccatgctacttatctacgtagccatgctctaggaagatcgtaccatt
gacgtcaataatgacgtatgttcccatagtaacgccaatagggactttccattgacgtcaatgg
gtggactatttacggtaaactgcccacttggcagtacatcaagtgtatcatatgccaagtacgc
cccctattgacgtcaatgacggtaaatggcccgcctggcattatgcccagtacatgaccttatg
ggactttcctacttggcagtacatctacgtattagtcatcgctattaccatggtcgaggtgagc
cccacgttctgcttcactctccccatctcccccccctccccacccccaattttgtatttattta
ttttttaattattttgtgcagcgatgggggcggggggggggggggggcgcgcgccaggcggggc
ggggcggggcgaggggcggggcggggcgaggcggagaggtgcggcggcagccaatcagagcggc
gcgctccgaaagtttccttttatggcgaggcggcggcggcggcggccctataaaaagcgaagcg
cgcggcgggcgggagtcgctgcgacgctgccttcgccccgtgccccgctccgccgccgcctcgc
gccgcccgccccggctctgactgaccgcgttactcccacaggtgagcgggcgggacggcccttc
tcctccgggctgtaattagcgcttggtttaatgacggcttgtttcttttctgtggctgcgtgaa
agccttgaggggctccgggagggccctttgtgcggggggagcggctcggggctgtccgcggggg
gacggctgccttcgggggggacggggcagggcggggttcggcttctggcgtgtgaccggcggct
ctagagcctctgctaaccatgttcatgccttcttctttttcctacagctcctgggcaacgtgct
ggttattgtgctgtctcatcattttggcaaagaattGGATCCGCCACCatgggcaagcccatcc
ccaaccccctgctgggcctggacagcaccGGCGGTGGAGGTTCCtccaaaaccatcgttctttc
ggtcggcgaggctactcgcactctgactgagatccagtccaccgcagaccgtcagatcttcgaa
gagaaggtcgggcctctggtgggtcggctgcgcctcacggcttcgctccgtcaaaacggagcca
agaccgcgtatcgcgtcaacctaaaactggatcaggcggacgtcgttgattgctccaccagcgt
ctgcggcgagcttccgaaagtgcgctacactcaggtatggtcgcacgacgtgacaatcgttgcg
aatagcaccgaggcctcgcgcaaatcgttgtacgatttgaccaagtccctcgtcgcgacctcgc
aggtcgaagatcttgtcgtcaaccttgtgccgctgggccgtggcggtggcggatctggcggcgg
tggtagcAATGATTTTGGCAATTACAACAATCAGTCTTCCAATTTTGGGCCGATGAAGGGAGGA
AACTTTGGAGGCAGGAGCTCTGGCCCTTATGGTGGTGGAGGCCAGTACTTTGCTAAACCACGGA
ACCAAGGTGGCTATGGCGGAGGTGGTTCTctgcctccacttgaaagactgacactgggctcagg
aggatctggtggttctgagggcagaggaagtcttctaacatgcggtgacgtggaggagaatccc
ggccctgCCatggattacaaggatgacgacgataagGGCGGAGGTGGTTCTtccaaaaccatcg
ttctttcggtcggcgaggctactcgcactctgactgagatccagtccaccgcagaccgtcagat
cttcgaagagaaggtcgggcctctggtgggtcggctgcgcctcacggcttcgctccgtcaaaac
ggagccaagaccgcgtatcgcgtcaacctaaaactggatcaggcggacgtcgttgattgctcca
ccagcgtctgcggcgagcttccgaaagtgcgctacactcaggtatggtcgcacgacgtgacaat
cgttgcgaatagcaccgaggcctcgcgcaaatcgttgtacgatttgaccaagtccctcgtcgcg
acctcgcaggtcgaagatcttgtcgtcaaccttgtgccgctgggccgtGGCGGAGGTGGTTCTa
agctgaaccctcctgatgagagtggccccggctgcatgagctgctgtgtgctctcctaagaatt
cgatatcaagcttatcgataatcaacctctggattacaaaatttgtgaaagattgactggtatt
cttaactatgttgctccttttacgctatgtggatacgctgctttaatgcctttgtatcatgcta
ttgcttcccgtatggctttcattttctcctccttgtataaatcctggttgctgtctctttatga
ggagttgtggcccgttgtcaggcaacgtggcgtggtgtgcactgtgtttgctgacgcaaccccc
actggttggggcattgccaccacctgtcagctcctttccgggactttcgctttccccctcccta
ttgccacggcggaactcatcgccgcctgccttgcccgctgctggacaggggctcggctgttggg
cactgacaattccgtggtgttgtcggggaaatcatcgtcctttccttggctgctcgcctatgtt
gccacctggattctgcgcgggacgtccttctgctacgtcccttcggccctcaatccagcggacc
ttccttcccgcggcctgctgccggctctgcggcctcttccgcgtcttcgccttcgccctcagac
gagtcggatctccctttgggccgcctccccgcatcgataccgagcgctgctcgagagatctacg
ggtggcatccctgtgacccctccccagtgcctctcctggccctggaagttgccactccagtgcc
caccagccttgtcctaataaaattaagttgcatcattttgtctgactaggtgtccttctataat
attatggggtggaggggggtggtatggagcaaggggcaagttgggaagacaacctgtagggcct
gcggggtctattgggaaccaagctggagtgcagtggcacaatcttggctcactgcaatctccgc
ctcctgggttcaagcgattctcctgcctcagcctcccgagttgttgggattccaggcatgcatg
accaggctcagctaatttttgtttttttggtagagacggggtttcaccatattggccaggctgg
tctccaactcctaatctcaggtgatctacccaccttggcctcccaaattgctgggattacaggc
gtgaaccactgctcccttccctgtccttctgattttgtaggtaaccacgtgcggaccgagcggc
cgcaggaacccctagtgatggagttggccactccctctctgcgcgctcgctcgctcactgaggc
cgggcgaccaaaggtcgcccgacgcccgggctttgcccgggcggcctcagtgagcgagcgagcg
cgcagctgcctgcaggggcgcctgatgcggtattttctccttacgcatctgtgcggtatttcac
accgcatacgtcaaagcaaccatagtacgcgccctgtagcggcgcattaagcgcggcgggtgtg
gtggttacgcgcagcgtgaccgctacacttgccagcgccttagcgcccgctcctttcgctttct
tcccttcctttctcgccacgttcgccggctttccccgtcaagctctaaatcgggggctcccttt
agggttccgatttagtgctttacggcacctcgaccccaaaaaacttgatttgggtgatggttca
cgtagtgggccatcgccctgatagacggtttttcgccctttgacgttggagtccacgttcttta
atagtggactcttgttccaaactggaacaacactcaactctatctcgggctattcttttgattt
ataagggattttgccgatttcggtctattggttaaaaaatgagctgatttaacaaaaatttaac
gcgaattttaacaaaatattaacgtttacaattttatggtgcactctcagtacaatctgctctg
atgccgcatagttaagccagccccgacacccgccaacacccgctgacgcgccctgacgggcttg
tctgctcccggcatccgcttacagacaagctgtgaccgtctccgggagctgcatgtgtcagagg
ttttcaccgtcatcaccgaaacgcgcgagacgaaagggcctcgtgatacgcctatttttatagg
ttaatgtcatgataataatggtttcttagacgtcaggtggcacttttcggggaaatgtgcgcgg
aacccctatttgtttatttttctaaatacattcaaatatgtatccgctcatgagacaataaccc
tgataaatgcttcaataatattgaaaaaggaagagtatgagtattcaacatttccgtgtcgccc
ttattcccttttttgcggcattttgccttcctgtttttgctcacccagaaacgctggtgaaagt
aaaagatgctgaagatcagttgggtgcacgagtgggttacatcgaactggatctcaacagcggt
aagatccttgagagttttcgccccgaagaacgttttccaatgatgagcacttttaaagttctgc
tatgtggcgcggtattatcccgtattgacgccgggcaagagcaactcggtcgccgcatacacta
ttctcagaatgacttggttgagtactcaccagtcacagaaaagcatcttacggatggcatgaca
gtaagagaattatgcagtgctgccataaccatgagtgataacactgcggccaacttacttctga
caacgatcggaggaccgaaggagctaaccgcttttttgcacaacatgggggatcatgtaactcg
ccttgatcgttgggaaccggagctgaatgaagccataccaaacgacgagcgtgacaccacgatg
cctgtagcaatggcaacaacgttgcgcaaactattaactggcgaactacttactctagcttccc
ggcaacaattaatagactggatggaggcggataaagttgcaggaccacttctgcgctcggccct
tccggctggctggtttattgctgataaatctggagccggtgagcgtgggtctcgcggtatcatt
gcagcactggggccagatggtaagccctcccgtatcgtagttatctacacgacggggagtcagg
caactatggatgaacgaaatagacagatcgctgagataggtgcctcactgattaagcattggta
actgtcagaccaagtttactcatatatactttagattgatttaaaacttcatttttaatttaaa
aggatctaggtgaagatcctttttgataatctcatgaccaaaatcccttaacgtgagttttcgt
tccactgagcgtcagaccccgtagaaaagatcaaaggatcttcttgagatcctttttttctgcg
cgtaatctgctgcttgcaaacaaaaaaaccaccgctaccagcggtggtttgtttgccggatcaa
gagctaccaactctttttccgaaggtaactggcttcagcagagcgcagataccaaatactgttc
ttctagtgtagccgtagttaggccaccacttcaagaactctgtagcaccgcctacatacctcgc
tctgctaatcctgttaccagtggctgctgccagtggcgataagtcgtgtcttaccgggttggac
tcaagacgatagttaccggataaggcgcagcggtcgggctgaacggggggttcgtgcacacagc
ccagcttggagcgaacgacctacaccgaactgagatacctacagcgtgagctatgagaaagcgc
cacgcttcccgaagggagaaaggcggacaggtatccggtaagcggcagggtcggaacaggagag
cgcacgagggagcttccagggggaaacgcctggtatctttatagtcctgtcgggtttcgccacc
tctgacttgagcgtcgatttttgtgatgctcgtcaggggggcggagcctatggaaaaacgccag
caacgcggcctttttacggttcctggccttttgctggccttttgctcacatgt

Plasmid #24 (see FIG. 42 for a map of the plasmid)

(SEQ ID NO: 115)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGTTAAGGATGCACCGACGGGACGTTCTATGAGGACGAATCTCCCGCTTATAAGATTCTA
TAAACTGTGCGGTCCTTCAATTGAAAAAAAAAAAAAAAAAAAAAAAAAAAAAAgggtcccatca
ttcatggcaagtggccgcggtcggcgtggactgtagaacactgccaatgccggtcccaagcccg
gataaaaGTGGAGGGTACAGTCCACGCtttttttctagactgcagagggccctgcgtatgagtg
caagtgggttttaggaccaggatgaggcggggtgggggtgcctacctgacgaccgaccccgacc
cactggacaagcacccaacccccattccccaaattgcgcatcccctatcagagagggggagggg
aaacaggatgcggcgaggcgcgtgcgcactgccagcttcagcaccgcggacagtgccttcgccc
ccgcctggcggcgcgcgccaccgccgcctcagcactgaaggcgcgctgacgtcactcgccggtc
ccccgcaaactccccttcccggccaccttggtcgcgtccgcgccgccgccggcccagccggacc
gcaccacgcgaggcgcgagataggggggcacgggcgcgaccatctgcgctgcggcgccggcgac
tcagcgctgcctcagtctgcggtgggcagcggaggagtcgtgtcgtgcctgagagcgcagtcga
gaaggtaccGGATCCGCCACCatgggcaagcccatccccaaccccctgctgggcctggacagca
CCGGCGGTGGAGGTTCCtccaaaaccatcgttctttcggtcggcgaggctactcgcactctgac
tgagatccagtccaccgcagaccgtcagatcttcgaagagaaggtcgggcctctggtgggtcgg
ctgcgcctcacggcttcgctccgtcaaaacggagccaagaccgcgtatcgcgtcaacctaaaac
tggatcaggcggacgtcgttgattgctccaccagcgtctgcggcgagcttccgaaagtgcgcta
cactcaggtatggtcgcacgacgtgacaatcgttgcgaatagcaccgaggcctcgcgcaaatcg
ttgtacgatttgaccaagtccctcgtcgcgacctcgcaggtcgaagatcttgtcgtcaaccttg
tgccgctgggccgtggcggtggcggatctggcggcggtggtagcAATGATTTTGGCAATTACAA
CAATCAGTCTTCCAATTTTGGGCCGATGAAGGGAGGAAACTTTGGAGGCAGGAGCTCTGGCCCT
TATGGTGGTGGAGGCCAGTACTTTGCTAAACCACGGAACCAAGGTGGCTATGGCGGAGGTGGTT
CTctgcctccacttgaaagactgacactgggctcaggaggatctggtggttctgagggcagagg
aagtcttctaacatgcggtgacgtggaggagaatcccggccctgCCatggattacaaggatgac
gacgataagGGCGGAGGTGGTTCTtccaaaaccatcgttctttcggtcggcgaggctactcgca
ctctgactgagatccagtccaccgcagaccgtcagatcttcgaagagaaggtcgggcctctggt
gggtcggctgcgcctcacggcttcgctccgtcaaaacggagccaagaccgcgtatcgcgtcaac
ctaaaactggatcaggcggacgtcgttgattgctccaccagcgtctgcggcgagcttccgaaag
tgcgctacactcaggtatggtcgcacgacgtgacaatcgttgcgaatagcaccgaggcctcgcg
caaatcgttgtacgatttgaccaagtccctcgtcgcgacctcgcaggtcgaagatcttgtcgtc
aaccttgtgccgctgggccgtGGCGGAGGTGGTTCTaagctgaaccctcctgatgagagtggcc
ccggctgcatgagctgctgtgtgctctcctaagaattcgatatcaagcttatcgataatcaacc
tctggattacaaaatttgtgaaagattgactggtattcttaactatgttgctccttttacgcta
tgtggatacgctgctttaatgcctttgtatcatgctattgcttcccgtatggctttcattttct
cctccttgtataaatcctggttgctgtctctttatgaggagttgtggcccgttgtcaggcaacg
tggcgtggtgtgcactgtgtttgctgacgcaacccccactggttggggcattgccaccacctgt
cagctcctttccgggactttcgctttccccctccctattgccacggcggaactcatcgccgcct
gccttgcccgctgctggacaggggctcggctgttgggcactgacaattccgtggtgttgtcggg
gaaatcatcgtcctttccttggctgctcgcctatgttgccacctggattctgcgcgggacgtcc
ttctgctacgtcccttcggccctcaatccagcggaccttccttcccgcggcctgctgccggctc
tgcggcctcttccgcgtcttcgccttcgccctcagacgagtcggatctccctttgggccgcctc
cccgcatcgataccgagcgctgctcgagagatctacgggtggcatccctgtgacccctccccag
tgcctctcctggccctggaagttgccactccagtgcccaccagccttgtcctaataaaattaag
ttgcatcattttgtctgactaggtgtccttctataatattatggggtggaggggggtggtatgg
agcaaggggcaagttgggaagacaacctgtagggcctgcggggtctattgggaaccaagctgga
gtgcagtggcacaatcttggctcactgcaatctccgcctcctgggttcaagcgattctcctgcc
tcagcctcccgagttgttgggattccaggcatgcatgaccaggctcagctaatttttgtttttt
tggtagagacggggtttcaccatattggccaggctggtctccaactcctaatctcaggtgatct
acccaccttggcctcccaaattgctgggattacaggcgtgaaccactgctcccttccctgtcct
tctgattttgtaggtaaccacgtgcggaccgagcggccgcaggaacccctagtgatggagttgg
ccactccctctctgcgcgctcgctcgctcactgaggccgggcgaccaaaggtcgcccgacgccc
gggctttgcccgggcggcctcagtgagcgagcgagcgcgcagctgcctgcaggggcgcctgatg
cggtattttctccttacgcatctgtgcggtatttcacaccgcatacgtcaaagcaaccatagta
cgcgccctgtagcggcgcattaagcgcggcgggtgtggtggttacgcgcagcgtgaccgctaca
cttgccagcgccttagcgcccgctcctttcgctttcttcccttcctttctcgccacgttcgccg
gctttccccgtcaagctctaaatcgggggctccctttagggttccgatttagtgctttacggca
cctcgaccccaaaaaacttgatttgggtgatggttcacgtagtgggccatcgccctgatagacg
gtttttcgccctttgacgttggagtccacgttctttaatagtggactcttgttccaaactggaa
caacactcaactctatctcgggctattcttttgatttataagggattttgccgatttcggtcta
ttggttaaaaaatgagctgatttaacaaaaatttaacgcgaattttaacaaaatattaacgttt
acaattttatggtgcactctcagtacaatctgctctgatgccgcatagttaagccagccccgac
acccgccaacacccgctgacgcgccctgacgggcttgtctgctcccggcatccgcttacagaca
agctgtgaccgtctccgggagctgcatgtgtcagaggttttcaccgtcatcaccgaaacgcgcg
agacgaaagggcctcgtgatacgcctatttttataggttaatgtcatgataataatggtttctt
agacgtcaggtggcacttttcggggaaatgtgcgcggaacccctatttgtttatttttctaaat
acattcaaatatgtatccgctcatgagacaataaccctgataaatgcttcaataatattgaaaa
aggaagagtatgagtattcaacatttccgtgtcgcccttattcccttttttgcggcattttgcc
ttcctgtttttgctcacccagaaacgctggtgaaagtaaaagatgctgaagatcagttgggtgc
acgagtgggttacatcgaactggatctcaacagcggtaagatccttgagagttttcgccccgaa
gaacgttttccaatgatgagcacttttaaagttctgctatgtggcgcggtattatcccgtattg
acgccgggcaagagcaactcggtcgccgcatacactattctcagaatgacttggttgagtactc
accagtcacagaaaagcatcttacggatggcatgacagtaagagaattatgcagtgctgccata
accatgagtgataacactgcggccaacttacttctgacaacgatcggaggaccgaaggagctaa
ccgcttttttgcacaacatgggggatcatgtaactcgccttgatcgttgggaaccggagctgaa
tgaagccataccaaacgacgagcgtgacaccacgatgcctgtagcaatggcaacaacgttgcgc
aaactattaactggcgaactacttactctagcttcccggcaacaattaatagactggatggagg
cggataaagttgcaggaccacttctgcgctcggcccttccggctggctggtttattgctgataa
atctggagccggtgagcgtgggtctcgcggtatcattgcagcactggggccagatggtaagccc
tcccgtatcgtagttatctacacgacggggagtcaggcaactatggatgaacgaaatagacaga
tcgctgagataggtgcctcactgattaagcattggtaactgtcagaccaagtttactcatatat
actttagattgatttaaaacttcatttttaatttaaaaggatctaggtgaagatcctttttgat
aatctcatgaccaaaatcccttaacgtgagttttcgttccactgagcgtcagaccccgtagaaa
agatcaaaggatcttcttgagatcctttttttctgcgcgtaatctgctgcttgcaaacaaaaaa
accaccgctaccagcggtggtttgtttgccggatcaagagctaccaactctttttccgaaggta
actggcttcagcagagcgcagataccaaatactgttcttctagtgtagccgtagttaggccacc
acttcaagaactctgtagcaccgcctacatacctcgctctgctaatcctgttaccagtggctgc
tgccagtggcgataagtcgtgtcttaccgggttggactcaagacgatagttaccggataaggcg
cagcggtcgggctgaacggggggttcgtgcacacagcccagcttggagcgaacgacctacaccg
aactgagatacctacagcgtgagctatgagaaagcgccacgcttcccgaagggagaaaggcgga
caggtatccggtaagcggcagggtcggaacaggagagcgcacgagggagcttccagggggaaac
gcctggtatctttatagtcctgtcgggtttcgccacctctgacttgagcgtcgatttttgtgat
gctcgtcaggggggcggagcctatggaaaaacgccagcaacgcggcctttttacggttcctggc
cttttgctggccttttgctcacatgt

Plasmid #25 (see FIG. 43 for a map of the plasmid)

(SEQ ID NO: 88)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggagggggcgggaaaccgcctaaccatgccgagtgcggccgcttgccatg
tgtatcggtccgGGAGCAGACGATATGGCGTCGCTCCcggtccgatactctgatgatgggtccc
CCTAGGGACGGGACGTTCTGCTAAGATCATTTCTCCCTGGGGCAGATTCTATAAACTGTGCGGT
CCTTCAATTGAAAAAAAAAAAAAAAAAAAAAAAAAAAAAAgggtcccatcattcatggcaagtg
gccgcggtcggcgtggactgtagaacactgccaatgccggtcccaagcccggataaaaGTGGAG
GGTACAGTCCACGCtttttttctagactgcagagggccctgcgtatgagtgcaagtgggtttta
ggaccaggatgaggcggggtgggggtgcctacctgacgaccgaccccgacccactggacaagca
cccaacccccattccccaaattgcgcatcccctatcagagagggggaggggaaacaggatgcgg
cgaggcgcgtgcgcactgccagcttcagcaccgcggacagtgccttcgcccccgcctggcggcg
cgcgccaccgccgcctcagcactgaaggcgcgctgacgtcactcgccggtcccccgcaaactcc
ccttcccggccaccttggtcgcgtccgcgccgccgccggcccagccggaccgcaccacgcgagg
cgcgagataggggggcacgggcgcgaccatctgcgctgcggcgccggcgactcagcgctgcctc
agtctgcggtgggcagcggaggagtcgtgtcgtgcctgagagcgcagtcgagaaggtaccGGAT
CCGCCACCatgggcaagcccatccccaaccccctgctgggcctggacagcaccGGCGGTGGAGG
TTCCtccaaaaccatcgttctttcggtcggcgaggctactcgcactctgactgagatccagtcc
accgcagaccgtcagatcttcgaagagaaggtcgggcctctggtgggtcggctgcgcctcacgg
cttcgctccgtcaaaacggagccaagaccgcgtatcgcgtcaacctaaaactggatcaggcgga
cgtcgttgattgctccaccagcgtctgcggcgagcttccgaaagtgcgctacactcaggtatgg
tcgcacgacgtgacaatcgttgcgaatagcaccgaggcctcgcgcaaatcgttgtacgatttga
ccaagtccctcgtcgcgacctcgcaggtcgaagatcttgtcgtcaaccttgtgccgctgggccg
tggcggtggcggatctggcggcggtggtagcAATGATTTTGGCAATTACAACAATCAGTCTTCC
AATTTTGGGCCGATGAAGGGAGGAAACTTTGGAGGCAGGAGCTCTGGCCCTTATGGTGGTGGAG
GCCAGTACTTTGCTAAACCACGGAACCAAGGTGGCTATGGCGGAGGTGGTTCTctgcctccact
tgaaagactgacactgggctcaggaggatctggtggttctgagggcagaggaagtcttctaaca
tgcggtgacgtggaggagaatcccggccctgCCatggtgagcaagggcgaggaggataacatgg
ccatcatcaaggagttcatgcgcttcaaggtgcacatggagggctccgtgaacggccacgagtt
cgagatcgagggcgagggcgagggccgcccctacgagggcacccagaccgccaagctgaaggtg
accaagggtggccccctgcccttcgcctgggacatcctgtcccctcagttcatgtacggctcca
aggcctacgtgaagcaccccgccgacatccccgactacttgaagctgtccttccccgagggctt
caagtgggagcgcgtgatgaacttcgaggacggcggcgtggtgaccgtgacccaggactcctcc
ctgcaggacggcgagttcatctacaaggtgaagctgcgcggcaccaacttcccctccgacggcc
ccgtaatgcagaagaagaccatgggctgggaggcctcctccgagcggatgtaccccgaggacgg
cgccctgaagggcgagatcaagcagaggctgaagctgaaggacggcggccactacgacgctgag
gtcaagaccacctacaaggccaagaagcccgtgcagctgcccggcgcctacaacgtcaacatca
agttggacatcacctcccacaacgaggactacaccatcgtggaacagtacgaacgcgccgaggg
ccgccactccaccggcggcatggacgagctgtacaagGGCGGAGGTGGTTCTtccaaaaccatc
gttctttcggtcggcgaggctactcgcactctgactgagatccagtccaccgcagaccgtcaga
tcttcgaagagaaggtcgggcctctggtgggtcggctgcgcctcacggcttcgctccgtcaaaa
cggagccaagaccgcgtatcgcgtcaacctaaaactggatcaggcggacgtcgttgattgctcc
accagcgtctgcggcgagcttccgaaagtgcgctacactcaggtatggtcgcacgacgtgacaa
tcgttgcgaatagcaccgaggcctcgcgcaaatcgttgtacgatttgaccaagtccctcgtcgc
gacctcgcaggtcgaagatcttgtcgtcaaccttgtgccgctgggccgtGGCGGAGGTGGTTCT
aagctgaaccctcctgatgagagtggccccggctgcatgagctgctgtgtgctctcctaagaat
tcgatatcaagcttatcgataatcaacctctggattacaaaatttgtgaaagattgactggtat
tcttaactatgttgctccttttacgctatgtggatacgctgctttaatgcctttgtatcatgct
attgcttcccgtatggctttcattttctcctccttgtataaatcctggttgctgtctctttatg
aggagttgtggcccgttgtcaggcaacgtggcgtggtgtgcactgtgtttgctgacgcaacccc
cactggttggggcattgccaccacctgtcagctcctttccgggactttcgctttccccctccct
attgccacggcggaactcatcgccgcctgccttgcccgctgctggacaggggctcggctgttgg
gcactgacaattccgtggtgttgtcggggaaatcatcgtcctttccttggctgctcgcctatgt
tgccacctggattctgcgcgggacgtccttctgctacgtcccttcggccctcaatccagcggac
cttccttcccgcggcctgctgccggctctgcggcctcttccgcgtcttcgccttcgccctcaga
cgagtcggatctccctttgggccgcctccccgcatcgataccgagcgctgctcgagagatctac
gggtggcatccctgtgacccctccccagtgcctctcctggccctggaagttgccactccagtgc
ccaccagccttgtcctaataaaattaagttgcatcattttgtctgactaggtgtccttctataa
tattatggggtggaggggggtggtatggagcaaggggcaagttgggaagacaacctgtagggcc
tgcggggtctattgggaaccaagctggagtgcagtggcacaatcttggctcactgcaatctccg
cctcctgggttcaagcgattctcctgcctcagcctcccgagttgttgggattccaggcatgcat
gaccaggctcagctaatttttgtttttttggtagagacggggtttcaccatattggccaggctg
gtctccaactcctaatctcaggtgatctacccaccttggcctcccaaattgctgggattacagg
cgtgaaccactgctcccttccctgtccttctgattttgtaggtaaccacgtgcggaccgagcgg
ccgcaggaacccctagtgatggagttggccactccctctctgcgcgctcgctcgctcactgagg
ccgggcgaccaaaggtcgcccgacgcccgggctttgcccgggcggcctcagtgagcgagcgagc
gcgcagctgcctgcaggggcgcctgatgcggtattttctccttacgcatctgtgcggtatttca
caccgcatacgtcaaagcaaccatagtacgcgccctgtagcggcgcattaagcgcggcgggtgt
ggtggttacgcgcagcgtgaccgctacacttgccagcgccttagcgcccgctcctttcgctttc
ttcccttcctttctcgccacgttcgccggctttccccgtcaagctctaaatcgggggctccctt
tagggttccgatttagtgctttacggcacctcgaccccaaaaaacttgatttgggtgatggttc
acgtagtgggccatcgccctgatagacggtttttcgccctttgacgttggagtccacgttcttt
aatagtggactcttgttccaaactggaacaacactcaactctatctcgggctattcttttgatt
tataagggattttgccgatttcggtctattggttaaaaaatgagctgatttaacaaaaatttaa
cgcgaattttaacaaaatattaacgtttacaattttatggtgcactctcagtacaatctgctct
gatgccgcatagttaagccagccccgacacccgccaacacccgctgacgcgccctgacgggctt
gtctgctcccggcatccgcttacagacaagctgtgaccgtctccgggagctgcatgtgtcagag
gttttcaccgtcatcaccgaaacgcgcgagacgaaagggcctcgtgatacgcctatttttatag
gttaatgtcatgataataatggtttcttagacgtcaggtggcacttttcggggaaatgtgcgcg
gaacccctatttgtttatttttctaaatacattcaaatatgtatccgctcatgagacaataacc
ctgataaatgcttcaataatattgaaaaaggaagagtatgagtattcaacatttccgtgtcgcc
cttattcccttttttgcggcattttgccttcctgtttttgctcacccagaaacgctggtgaaag
taaaagatgctgaagatcagttgggtgcacgagtgggttacatcgaactggatctcaacagcgg
taagatccttgagagttttcgccccgaagaacgttttccaatgatgagcacttttaaagttctg
ctatgtggcgcggtattatcccgtattgacgccgggcaagagcaactcggtcgccgcatacact
attctcagaatgacttggttgagtactcaccagtcacagaaaagcatcttacggatggcatgac
agtaagagaattatgcagtgctgccataaccatgagtgataacactgcggccaacttacttctg
acaacgatcggaggaccgaaggagctaaccgcttttttgcacaacatgggggatcatgtaactc
gccttgatcgttgggaaccggagctgaatgaagccataccaaacgacgagcgtgacaccacgat
gcctgtagcaatggcaacaacgttgcgcaaactattaactggcgaactacttactctagcttcc
cggcaacaattaatagactggatggaggcggataaagttgcaggaccacttctgcgctcggccc
ttccggctggctggtttattgctgataaatctggagccggtgagcgtgggtctcgcggtatcat
tgcagcactggggccagatggtaagccctcccgtatcgtagttatctacacgacggggagtcag
gcaactatggatgaacgaaatagacagatcgctgagataggtgcctcactgattaagcattggt
aactgtcagaccaagtttactcatatatactttagattgatttaaaacttcatttttaatttaa
aaggatctaggtgaagatcctttttgataatctcatgaccaaaatcccttaacgtgagttttcg
ttccactgagcgtcagaccccgtagaaaagatcaaaggatcttcttgagatcctttttttctgc
gcgtaatctgctgcttgcaaacaaaaaaaccaccgctaccagcggtggtttgtttgccggatca
agagctaccaactctttttccgaaggtaactggcttcagcagagcgcagataccaaatactgtt
cttctagtgtagccgtagttaggccaccacttcaagaactctgtagcaccgcctacatacctcg
ctctgctaatcctgttaccagtggctgctgccagtggcgataagtcgtgtcttaccgggttgga
ctcaagacgatagttaccggataaggcgcagcggtcgggctgaacggggggttcgtgcacacag
cccagcttggagcgaacgacctacaccgaactgagatacctacagcgtgagctatgagaaagcg
ccacgcttcccgaagggagaaaggcggacaggtatccggtaagcggcagggtcggaacaggaga
gcgcacgagggagcttccagggggaaacgcctggtatctttatagtcctgtcgggtttcgccac
ctctgacttgagcgtcgatttttgtgatgctcgtcaggggggcggagcctatggaaaaacgcca
gcaacgcggcctttttacggttcctggccttttgctggccttttgctcacatgt

Plasmid #26 (see FIG. 44 for a map of the plasmid)

(SEQ ID NO: 116)
cctgcaggcagctgcgcgctcgctcgctcactgaggccgcccgggcgtcgggcgacctttggtc
gcccggcctcagtgagcgagcgagcgcgcagagagggagtggccaactccatcactaggggttc
ctgcggccgcacgcgtgagggcctatttcccatgattccttcatatttgcatatacgatacaag
gctgttagagagataattggaattaatttgactgtaaacacaaagatattagtacaaaatacgt
gacgtagaaagtaataatttcttgggtagtttgcagttttaaaattatgttttaaaatggacta
tcatatgcttaccgtaacttgaaagtatttcgatttcttggctttatatatcttgtggaaagga
cgaaacaccgtgctcgcttcggcagcacatatactagtcgacgggccgcactcgccggtcccaa
gcccggataaaatgggggcgggcgggaaaccgcctaaccatgccgagtgcggccgcttgccatg
tgtatcggtccgGGAGCAGACGATATGGCGTCGCTCCcggtccgatactctgatgatgggtccc
CCTAGGGACGGGACGTTCTGCTAAGATCATTTCTCCCTGGGGCAGATTCTATAAACTGTGCGGT
CCTTCAATTGAAAAAAAAAAAAAAAAAAAAAAAAAAAAAAgggtcccatcattcatggcaagtg
gccgcggtcggcgtggactgtagaacactgccaatgccggtcccaagcccggataaaaGTGGAG
GGTACAGTCCACGCtttttttctagactgcagagggccctcaagtgccacctgacgtctcccta
tcagtgatagagaagtcgacacgtctcgagctccctatcagtgatagagaaggtacgtctagaa
cgtctccctatcagtgatagagaagtcgacacgtctcgagctccctatcagtgatagagaaggt
acgtctagaacgtctccctatcagtgatagagaagtcgacacgtctcgagctccctatcagtga
tagagaaggtacgtctagaacgtctccctatcagtgatagagaagtcgacacgtctcgagctcc
ctatcagtgatagagaagctaccccctatataagcagagctcgtttagtgaaccgtcagatcgc
ctggagacgccatccacgctgttttgacctccatagaagacaccgggaccgatccagcctGGAT
CCGCCACCatgggcaagcccatccccaaccccctgctgggcctggacagcaccGGCGGTGGAGG
TTCCtccaaaaccatcgttctttcggtcggcgaggctactcgcactctgactgagatccagtcc
accgcagaccgtcagatcttcgaagagaaggtcgggcctctggtgggtcggctgcgcctcacgg
cttcgctccgtcaaaacggagccaagaccgcgtatcgcgtcaacctaaaactggatcaggcgga
cgtcgttgattgctccaccagcgtctgcggcgagcttccgaaagtgcgctacactcaggtatgg
tcgcacgacgtgacaatcgttgcgaatagcaccgaggcctcgcgcaaatcgttgtacgatttga
ccaagtccctcgtcgcgacctcgcaggtcgaagatcttgtcgtcaaccttgtgccgctgggccg
tggcggtggcggatctggcggcggtggtagcAATGATTTTGGCAATTACAACAATCAGTCTTCC
AATTTTGGGCCGATGAAGGGAGGAAACTTTGGAGGCAGGAGCTCTGGCCCTTATGGTGGTGGAG
GCCAGTACTTTGCTAAACCACGGAACCAAGGTGGCTATGGCGGAGGTGGTTCTctgcctccact
tgaaagactgacactgggctcaggaggatctggtggttctgagggcagaggaagtcttctaaca
tgcggtgacgtggaggagaatcccggccctgCCatggtgagcaagggcgaggaggataacatgg
ccatcatcaaggagttcatgcgcttcaaggtgcacatggagggctccgtgaacggccacgagtt
cgagatcgagggcgagggcgagggccgcccctacgagggcacccagaccgccaagctgaaggtg
accaagggtggccccctgcccttcgcctgggacatcctgtcccctcagttcatgtacggctcca
aggcctacgtgaagcaccccgccgacatccccgactacttgaagctgtccttccccgagggctt
caagtgggagcgcgtgatgaacttcgaggacggcggcgtggtgaccgtgacccaggactcctcc
ctgcaggacggcgagttcatctacaaggtgaagctgcgcggcaccaacttcccctccgacggcc
ccgtaatgcagaagaagaccatgggctgggaggcctcctccgagcggatgtaccccgaggacgg
cgccctgaagggcgagatcaagcagaggctgaagctgaaggacggcggccactacgacgctgag
gtcaagaccacctacaaggccaagaagcccgtgcagctgcccggcgcctacaacgtcaacatca
agttggacatcacctcccacaacgaggactacaccatcgtggaacagtacgaacgcgccgaggg
ccgccactccaccggcggcatggacgagctgtacaagGGCGGAGGTGGTTCTtccaaaaccatc
gttctttcggtcggcgaggctactcgcactctgactgagatccagtccaccgcagaccgtcaga
tcttcgaagagaaggtcgggcctctggtgggtcggctgcgcctcacggcttcgctccgtcaaaa
cggagccaagaccgcgtatcgcgtcaacctaaaactggatcaggcggacgtcgttgattgctcc
accagcgtctgcggcgagcttccgaaagtgcgctacactcaggtatggtcgcacgacgtgacaa
tcgttgcgaatagcaccgaggcctcgcgcaaatcgttgtacgatttgaccaagtccctcgtcgc
gacctcgcaggtcgaagatcttgtcgtcaaccttgtgccgctgggccgtGGCGGAGGTGGTTCT
aagctgaaccctcctgatgagagtggccccggctgcatgagctgctgtgtgctctcctaagaat
tcgatatcaagcttatcgataatcaacctctggattacaaaatttgtgaaagattgactggtat
tcttaactatgttgctccttttacgctatgtggatacgctgctttaatgcctttgtatcatgct
attgcttcccgtatggctttcattttctcctccttgtataaatcctggttgctgtctctttatg
aggagttgtggcccgttgtcaggcaacgtggcgtggtgtgcactgtgtttgctgacgcaacccc
cactggttggggcattgccaccacctgtcagctcctttccgggactttcgctttccccctccct
attgccacggcggaactcatcgccgcctgccttgcccgctgctggacaggggctcggctgttgg
gcactgacaattccgtggtgttgtcggggaaatcatcgtcctttccttggctgctcgcctatgt
tgccacctggattctgcgcgggacgtccttctgctacgtcccttcggccctcaatccagcggac
cttccttcccgcggcctgctgccggctctgcggcctcttccgcgtcttcgccttcgccctcaga
cgagtcggatctccctttgggccgcctccccgcatcgataccgagcgctgctcgagagatctac
gggtggcatccctgtgacccctccccagtgcctctcctggccctggaagttgccactccagtgc
ccaccagccttgtcctaataaaattaagttgcatcattttgtctgactaggtgtccttctataa
tattatggggtggaggggggtggtatggagcaaggggcaagttgggaagacaacctgtagggcc
tgcggggtctattgggaaccaagctggagtgcagtggcacaatcttggctcactgcaatctccg
cctcctgggttcaagcgattctcctgcctcagcctcccgagttgttgggattccaggcatgcat
gaccaggctcagctaatttttgtttttttggtagagacggggtttcaccatattggccaggctg
gtctccaactcctaatctcaggtgatctacccaccttggcctcccaaattgctgggattacagg
cgtgaaccactgctcccttccctgtccttctgattttgtaggtaaccacgtgcggaccgagcgg
ccgcaggaacccctagtgatggagttggccactccctctctgcgcgctcgctcgctcactgagg
ccgggcgaccaaaggtcgcccgacgcccgggctttgcccgggcggcctcagtgagcgagcgagc
gcgcagctgcctgcaggggcgcctgatgcggtattttctccttacgcatctgtgcggtatttca
caccgcatacgtcaaagcaaccatagtacgcgccctgtagcggcgcattaagcgcggcgggtgt
ggtggttacgcgcagcgtgaccgctacacttgccagcgccttagcgcccgctcctttcgctttc
ttcccttcctttctcgccacgttcgccggctttccccgtcaagctctaaatcgggggctccctt
tagggttccgatttagtgctttacggcacctcgaccccaaaaaacttgatttgggtgatggttc
acgtagtgggccatcgccctgatagacggtttttcgccctttgacgttggagtccacgttcttt
aatagtggactcttgttccaaactggaacaacactcaactctatctcgggctattcttttgatt
tataagggattttgccgatttcggtctattggttaaaaaatgagctgatttaacaaaaatttaa
cgcgaattttaacaaaatattaacgtttacaattttatggtgcactctcagtacaatctgctct
gatgccgcatagttaagccagccccgacacccgccaacacccgctgacgcgccctgacgggctt
gtctgctcccggcatccgcttacagacaagctgtgaccgtctccgggagctgcatgtgtcagag
gttttcaccgtcatcaccgaaacgcgcgagacgaaagggcctcgtgatacgcctatttttatag
gttaatgtcatgataataatggtttcttagacgtcaggtggcacttttcggggaaatgtgcgcg
gaacccctatttgtttatttttctaaatacattcaaatatgtatccgctcatgagacaataacc
ctgataaatgcttcaataatattgaaaaaggaagagtatgagtattcaacatttccgtgtcgcc
cttattcccttttttgcggcattttgccttcctgtttttgctcacccagaaacgctggtgaaag
taaaagatgctgaagatcagttgggtgcacgagtgggttacatcgaactggatctcaacagcgg
taagatccttgagagttttcgccccgaagaacgttttccaatgatgagcacttttaaagttctg
ctatgtggcgcggtattatcccgtattgacgccgggcaagagcaactcggtcgccgcatacact
attctcagaatgacttggttgagtactcaccagtcacagaaaagcatcttacggatggcatgac
agtaagagaattatgcagtgctgccataaccatgagtgataacactgcggccaacttacttctg
acaacgatcggaggaccgaaggagctaaccgcttttttgcacaacatgggggatcatgtaactc
gccttgatcgttgggaaccggagctgaatgaagccataccaaacgacgagcgtgacaccacgat
gcctgtagcaatggcaacaacgttgcgcaaactattaactggcgaactacttactctagcttcc
cggcaacaattaatagactggatggaggcggataaagttgcaggaccacttctgcgctcggccc
ttccggctggctggtttattgctgataaatctggagccggtgagcgtgggtctcgcggtatcat
tgcagcactggggccagatggtaagccctcccgtatcgtagttatctacacgacggggagtcag
gcaactatggatgaacgaaatagacagatcgctgagataggtgcctcactgattaagcattggt
aactgtcagaccaagtttactcatatatactttagattgatttaaaacttcatttttaatttaa
aaggatctaggtgaagatcctttttgataatctcatgaccaaaatcccttaacgtgagttttcg
ttccactgagcgtcagaccccgtagaaaagatcaaaggatcttcttgagatcctttttttctgc
gcgtaatctgctgcttgcaaacaaaaaaaccaccgctaccagcggtggtttgtttgccggatca
agagctaccaactctttttccgaaggtaactggcttcagcagagcgcagataccaaatactgtt
cttctagtgtagccgtagttaggccaccacttcaagaactctgtagcaccgcctacatacctcg
ctctgctaatcctgttaccagtggctgctgccagtggcgataagtcgtgtcttaccgggttgga
ctcaagacgatagttaccggataaggcgcagcggtcgggctgaacggggggttcgtgcacacag
cccagcttggagcgaacgacctacaccgaactgagatacctacagcgtgagctatgagaaagcg
ccacgcttcccgaagggagaaaggcggacaggtatccggtaagcggcagggtcggaacaggaga
gcgcacgagggagcttccagggggaaacgcctggtatctttatagtcctgtcgggtttcgccac
ctctgacttgagcgtcgatttttgtgatgctcgtcaggggggcggagcctatggaaaaacgcca
gcaacgcggcctttttacggttcctggccttttgctggccttttgctcacatgt

The following tables providing amino acid and polynucleotide sequences for elements used in the above-listed plasmid sequences:

TABLE 3
Polynucleotide sequences for elements used in the examples.

| Element Name | Nucleotide Sequence |
|---|---|
| λ N peptide | gacgcacaaacacgacgacgtgagcgtcgcgctgagaaacaagctca |
| | atggaaagctgcaaac (SEQ ID NO: 32) |
| WPRE (woodchuck hepatitis | aatcaacctctggattacaaaatttgtgaaagattgactggtattct |
| virus posttranscriptional | taactatgttgctccttttacgctatgtggatacgctgctttaatgc |
| regulatory element) | ctttgtatcatgctattgcttcccgtatggctttcattttctcctcc |
| | ttgtataaatcctggttgctgtctctttatgaggagttgtggcccgt |
| | tgtcaggcaacgtggcgtggtgtgcactgtgtttgctgacgcaaccc |
| | ccactggttggggcattgccaccacctgtcagctcctttccgggact |
| | ttcgctttccccctccctattgccacggcggaactcatcgccgcctg |
| | ccttgcccgctgctggacaggggctcggctgttgggcactgacaatt |
| | ccgtggtgttgtcggggaaatcatcgtcctttccttggctgctcgcc |
| | tatgttgccacctggattctgcgcgggacgtccttctgctacgtccc |
| | ttcggccctcaatccagcggaccttccttcccgcggcctgctgccgg |
| | ctctgcggcctcttccgcgtcttcgccttcgccctcagacgagtcgg |
| | atctccctttgggccgcctccccgc (SEQ ID NO: 117) |
| VAMP2A | atgtcggctaccgctgccaccgtcccgcctgccgccccggccggcga |
| | gggtggcccccctgcacctcctccaaaccttactagtaacaggagac |
| | tgcagcagacccaggcccaggtggatgaggtggtggacatcatgagg |
| | gtgaatgtggacaaggtcctggagcgggaccagaagttgtcggagct |
| | ggatgaccgtgcagatgccctccaggcaggggcctcccagtttgaaa |
| | caagtgcagccaagctcaagcgcaaatactggtggaaaaacctcaag |
| | atgatgatcatcttgggagtgatctgcgccatcatcctcatcatcat |
| | catcgtttacttcagcact (SEQ ID NO: 50) |
| V5 tag | ggcaagcccatccccaaccccctgctgggcctggacagcacc (SEQ |
| | ID NO: 118) |
| U6 promoter (RNA polymerase | gagggcctatttcccatgattccttcatatttgcatatacgatacaa |
| III promoter for human U6 | ggctgttagagagataattggaattaatttgactgtaaacacaaaga |
| snRNA) | tattagtacaaaatacgtgacgtagaaagtaataatttcttgggtag |
| | tttgcagttttaaaattatgttttaaaatggactatcatatgcttac |
| | cgtaacttgaaagtatttcgatttcttggctttatatatcttgtgga |
| | aaggac (SEQ ID NO: 48) |
| tet operator (bound_moiety is | tccctatcagtgatagaga (SEQ ID NO: 119) |
| tetracycline repressor TetR; | |
| bacterial operator O2 for the | |
| tetR and tetA genes) | |
| T2A | gagggcagaggaagtcttctaacatgcggtgacgtggaggagaatcc |
| | cggccct (SEQ ID NO: 120) |
| SV40 promoter (SV40 enhancer | GTGTGTCAGTTAGGGTGTGGAAAGTCCCCAGGCTCCCCAGCAGGCAG |
| and early promoter) | AAGTATGCAAAGCATGCATCTCAATTAGTCAGCAACCAGGTGTGGAA |
| | AGTCCCCAGGCTCCCCAGCAGGCAGAAGTATGCAAAGCATGCATCTC |
| | AATTAGTCAGCAACCATAGTCCCGCCCCTAACTCCGCCCATCCCGCC |
| | CCTAACTCCGCCCAGTTCCGCCCATTCTCCGCCCCATGGCTGACTAA |
| | TTTTTTTTATTTATGCAGAGGCCGAGGCCGCCTCTGCCTCTGAGCTA |
| | TTCCAGAAGTAGTGAGGAGGCTTTTTTGGAGGCCTAGGCTTTTGCAA |
| | A (SEQ ID NO: 121) |
| SV40 poly(A) signal (SV40 | AACTTGTTTATTGCAGCTTATAATGGTTACAAATAAAGCAATAGCAT |
| polyadenylation signal) | CACAAATTTCACAAATAAAGCATTTTTTTCACTGCATTCTAGTTGTG |
| | GTTTGTCCAAACTCATCAATGTATCTTA (SEQ ID NO: 122) |
| SV40 ori (SV40 origin of | ATCCCGCCCCTAACTCCGCCCAGTTCCGCCCATTCTCCGCCCCATGG |
| replication) | CTGACTAATTTTTTTTATTTATGCAGAGGCCGAGGCCGCCTCTGCCT |
| | CTGAGCTATTCCAGAAGTAGTGAGGAGGCTTTTTTGGAGGCC (SEQ |
| | ID NO: 123) |
| SV40 NLS | cccaagaagaagaggaaggtg (SEQ ID NO: 124) |
| Rat SYPH | gacgtggtgaatcagctggtggctgggggtcagttccgggtggtcaa |
| | ggagccccttggcttcgtgaaggtgctgcagtgggtctttgccatct |
| | tcgcctttgctacgtgtggcagctacaccggggagcttcggctgagc |
| | gtggagtgtgccaacaagacggagagtgccctcaacatcgaagttga |
| | attcgagtaccccttcaggctgcaccaagtgtactttgatgcaccct |
| | cctgcgtcaaagggggcactaccaagatcttcctggttggggactac |
| | tcctcgtcggctgaattctttgtcaccgtggctgtgtttgccttcct |
| | ctactccatgggggccctggccacctacatcttcctgcagaacaagt |
| | accgagagaacaacaaagggcctatgatggactttctggctacagcc |
| | gtgttcgctttcatgtggctagttagttcatcagcctgggccaaagg |
| | cctgtccgatgtgaagatggccacggacccagagaacattatcaagg |
| | agatgcccatgtgccgccagacagggaacacatgcaaggaactgagg |
| | gaccctgtgacttcaggactcaacacctcagtggtgtttggcttcct |
| | gaacctggtgctctgggttggcaacttatggttcgtgttcaaggaga |
| | caggctgggcagccccattcatgcgcgcacctccaggcgccccggaa |
| | aagcaaccagcacctggcgatgcctacggcgatgcgggctacgggca |
| | gggccccggaggctatgggccccaggactcctacgggcctcagggtg |
| | gttatcaacccgattacgggcagccagccagcggtggcggtggctac |
| | gggcctcagggcgactatgggcagcaaggctatggccaacagggtgc |
| | gcccacctccttctccaatcagatg (SEQ ID NO: 125) |
| rat Homerlc (Q9Z214) | atgggggaacaacctatcttcagcactcgagctcatgtcttccagat |
| | cgacccaaacacaaagaagaactgggtacccaccagcaagcatgcag |
| | ttactgtgtcttatttctatgacagcacaaggaatgtgtataggata |
| | atcagtttagacggctcaaaggcaataataaatagcaccatcactcc |
| | aaacatgacatttactaaaacatctcaaaagtttggccaatgggctg |
| | atagccgggcaaacactgtttatggactgggattctcctctgagcat |
| | catctctcaaaatttgcagaaaagtttcaggaatttaaagaagctgc |
| | tcggctggcaaaggagaagtcgcaggagaagatggaactgaccagta |
| | ccccttcacaggaatcagcaggaggagatcttcagtctcctttaaca |
| | ccagaaagtatcaatgggacagatgatgagagaacacccgatgtgac |
| | acagaactcagagccaagggctgagccagctcagaatgcattgccat |
| | tttcacatagtgccggggatcgaacccagggcctctctcatgctagt |
| | tcagccatcagcaaacactgggaggctgaactagccacgctcaaggg |
| | gaacaatgccaagctcaccgcagcgctgctggagtccactgccaacg |
| | tgaagcagtggaagcaacagctggctgcctaccaggaggaggcagag |
| | cggctgcacaagcgggtcacggagctggaatgtgttagtagtcaagc |
| | aaacgcggtgcacagccacaagacagagctgagtcagacagtgcagg |
| | agctggaagagaccctaaaagtaaaggaagaggaaatagaaagatta |
| | aaacaagaaattgataacgccagagaacttcaagaacagagggactc |
| | tttgactcagaaactacaggaagttgagattcgaaataaagacctgg |
| | aggggcagctgtcggagctggagcagcgcctggagaagagccagagc |
| | gagcaggacgctttccgcagtaacctgaagactctcctagagattct |
| | ggacgggaaaatatttgaactaacagaattgcgggataatttggcca |
| | agctactagaatgcagctaa (SEQ ID NO: 29) |
| PSD95.FingR | atgctcgaagtcaaggaagcatcaccaaccagcatccagatcagctg |
| | ggtgctccacttgcgccacgttcgctactaccgcatcacctacggtg |
| | aaactggtggcaatagccctgtccaggaattcaccgtgcctggcagc |
| | aagtccactgctaccatcagcggcctgaaacctggtgtcgactatac |
| | catcacggtgtacgccgtcacgatcttcagcgcctaccgctccgcct |
| | ggccgccgatctccatcaactaccgcaccggaacc (SEQ ID NO: |
| | 43) |
| PSD95.FingR | atgctcgaagtcaaggaagcatcaccaaccagcatccagatcagctg |
| | ggtgctccacttgcgccacgttcgctactaccgcatcacctacggtg |
| | aaactggtggcaatagccctgtccaggaattcaccgtgcctggcagc |
| | aagtccactgctaccatcagcggcctgaaacctggtgtcgactatac |
| | catcacggtgtacgccgtcacgatcttcagcgcctaccgctccgcct |
| | ggccgccgatctccatcaactaccgcaccggaacc (SEQ ID NO: |
| | 43) |
| PP7cp-2 | TCCAAAACCATCGTTCTTTCGGTCGGCGAGGCTACTCGCACTCTGAC |
| | TGAGATCCAGTCCACCGCAGACCGTCAGATCTTCGAAGAGAAGGTCG |
| | GGCCTCTGGTGGGTCGGCTGCGCCTCACGGCTTCGCTCCGTCAAAAC |
| | GGAGCCAAGACCGCGTATCGCGTCAACCTAAAACTGGATCAGGCGGA |
| | CGTCGTTGATTGCTCCACCAGCGTCTGCGGCGAGCTTCCGAAAGTGC |
| | GCTACACTCAGGTATGGTCGCACGACGTGACAATCGTTGCGAATAGC |
| | ACCGAGGCCTCGCGCAAATCGTTGTACGATTTGACCAAGTCCCTCGT |
| | CGCGACCTCGCAGGTCGAAGATCTTGTCGTCAACCTTGTGCCGCTGG |
| | GCCGT (SEQ ID NO: 39) |
| PP7cp-1 | ATGTCCAAAACCATCGTTCTTTCGGTCGGCGAGGCTACTCGCACTCT |
| | GACTGAGATCCAGTCCACCGCAGACCGTCAGATCTTCGAAGAGAAGG |
| | TCGGGCCTCTGGTGGGTCGGCTGCGCCTCACGGCTTCGCTCCGTCAA |
| | AACGGAGCCAAGACCGCGTATCGCGTCAACCTAAAACTGGATCAGGC |
| | GGACGTCGTTGATTGCTCCACCAGCGTCTGCGGCGAGCTTCCGAAAG |
| | TGCGCTACACTCAGGTATGGTCGCACGACGTGACAATCGTTGCGAAT |
| | AGCACCGAGGCCTCGCGCAAATCGTTGTACGATTTGACCAAGTCCCT |
| | CGTCGCGACCTCGCAGGTCGAAGATCTTGTCGTCAACCTTGTGCCGC |
| | TGGGCCGT (SEQ ID NO: 126) |
| PP7cp | atgtccaaaaccatcgttctttcggtcggcgaggctactcgcactct |
| | gactgagatccagtccaccgcagaccgtcagatcttcgaagagaagg |
| | tcgggcctctggtgggtcggctgcgcctcacggcttcgctccgtcaa |
| | aacggagccaagaccgcgtatcgcgtcaacctaaaactggatcaggc |
| | ggacgtcgttgattgctccaccagcgtctgcggcgagcttccgaaag |
| | tgcgctacactcaggtatggtcgcacgacgtgacaatcgttgcgaat |
| | agcaccgaggcctcgcgcaaatcgttgtacgatttgaccaagtccct |
| | cgtcgcgacctcgcaggtcgaagatcttgtcgtcaaccttgtgccgc |
| | tgggccgt (SEQ ID NO: 126) |
| PP7 version 2 | CCAGCAGAGCATATGGGCTCGCTGG (SEQ ID NO: 41) |
| PP7 version 1 | GGAGCAGACGATATGGCGTCGCTCC (SEQ ID NO: 40) |
| ori (right; high-copy-number | ttgagatcctttttttctgcgcgtaatctgctgcttgcaaacaaaaa |
| ColE1/pMB1/pBR322/pUC | aaccaccgctaccagcggtggtttgtttgccggatcaagagctacca |
| origin of replication) | actctttttccgaaggtaactggcttcagcagagcgcagataccaaa |
| | tactgttcttctagtgtagccgtagttaggccaccacttcaagaact |
| | ctgtagcaccgcctacatacctcgctctgctaatcctgttaccagtg |
| | gctgctgccagtggcgataagtcgtgtcttaccgggttggactcaag |
| | acgatagttaccggataaggcgcagcggtcgggctgaacggggggtt |
| | cgtgcacacagcccagcttggagcgaacgacctacaccgaactgaga |
| | tacctacagcgtgagctatgagaaagcgccacgcttcccgaagggag |
| | aaaggcggacaggtatccggtaagcggcagggtcggaacaggagagc |
| | gcacgagggagcttccagggggaaacgcctggtatctttatagtcct |
| | gtcgggtttcgccacctctgacttgagcgtcgatttttgtgatgctc |
| | gtcaggggggcggagcctatggaaa (SEQ ID NO: 127) |
| ori (left; high-copy-number | TTTCCATAGGCTCCGCCCCCCTGACGAGCATCACAAAAATCGACGCT |
| ColE1/pMB1/pBR322/pUC | CAAGTCAGAGGTGGCGAAACCCGACAGGACTATAAAGATACCAGGCG |
| origin of replication) | TTTCCCCCTGGAAGCTCCCTCGTGCGCTCTCCTGTTCCGACCCTGCC |
| | GCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTT |
| | CGCTCCAAGCTGGGCTGTGTGCACGAACCCCCCGTTCAGCCCGACCG |
| | CTGCGCCTTATCCGGTAACTATCGTCTTGAGTCCAACCCGGTAAGAC |
| | ACGACTTATCGCCACTGGCAGCAGCCACTGGTAACAGGATTAGCAGA |
| | GCGAGGTATGTAGGCGGTGCTACAGAGTTCTTGAAGTGGTGGCCTAA |
| | CTACGGCTACACTAGAAGAACAGTATTTGGTATCTGCGCTCTGCTGA |
| | AGCCAGTTACCTTCGGAAAAAGAGTTGGTAGCTCTTGATCCGGCAAA |
| | CAAACCACCGCTGGTAGCGGTTTTTTTGTTTGCAAGCAGCAGATTAC |
| | GCGCAGAAAAAAAGGATCTCAA (SEQ ID NO: 128) |
| N-Palmitoylation | Atgctgtgctgcatcagaagaactaaaccggttgagaagaatgaaga |
| | ggccgatcaggagctgcagtcgacggtgccgcgggcccgggatccac |
| | cggtcgccacc (SEQ ID NO: 129) |
| NES (nuclear export signal | ctgcctccacttgaaagactgacactg (SEQ ID NO: 130) |
| from the HIV Rev protein; see | |
| Fischer U, et al. Cell. 1995 Aug. | |
| 11; 82(3): 475-83. doi: | |
| 10.1016/0092-8674(95) | |
| 90436-0. PMID: 7543368) | |
| NeoR/KanR (aminoglycoside | ATGATTGAACAAGATGGATTGCACGCAGGTTCTCCGGCCGCTTGGGT |
| phosphotransferase from Tn5, | GGAGAGGCTATTCGGCTATGACTGGGCACAACAGACAATCGGCTGCT |
| which confers resistance to | CTGATGCCGCCGTGTTCCGGCTGTCAGCGCAGGGGCGCCCGGTTCTT |
| neomycin, kanamycin, and | TTTGTCAAGACCGACCTGTCCGGTGCCCTGAATGAACTGCAGGACGA |
| G418 (Geneticin)) | GGCAGCGCGGCTATCGTGGCTGGCCACGACGGGCGTTCCTTGCGCAG |
| | CTGTGCTCGACGTTGTCACTGAAGCGGGAAGGGACTGGCTGCTATTG |
| | GGCGAAGTGCCGGGGCAGGATCTCCTGTCATCTCACCTTGCTCCTGC |
| | CGAGAAAGTATCCATCATGGCTGATGCAATGCGGCGGCTGCATACGC |
| | TTGATCCGGCTACCTGCCCATTCGACCACCAAGCGAAACATCGCATC |
| | GAGCGAGCACGTACTCGGATGGAAGCCGGTCTTGTCGATCAGGATGA |
| | TCTGGACGAAGAGCATCAGGGGCTCGCGCCAGCCGAACTGTTCGCCA |
| | GGCTCAAGGCGCGCATGCCCGACGGCGAGGATCTCGTCGTGACCCAT |
| | GGCGATGCCTGCTTGCCGAATATCATGGTGGAAAATGGCCGCTTTTC |
| | TGGATTCATCGACTGTGGCCGGCTGGGTGTGGCGGACCGCTATCAGG |
| | ACATAGCGTTGGCTACCCGTGATATTGCTGAAGAGCTTGGCGGCGAA |
| | TGGGCTGACCGCTTCCTCGTGCTTTACGGTATCGCCGCTCCCGATTC |
| | GCAGCGCATCGCCTTCTATCGCCTTCTTGACGAGTTCTTCTGA |
| | (SEQ ID NO: 131) |
| myr (N-myristoylation signal | ATGGGGTCTTCAAAATCTAAACCAAAGGACCCCAGCCAGCGC (SEQ |
| from Src kinase; see Pellman D, | ID NO: 132) |
| et al. Nature. 1985 Mar. 28- | |
| April 3; 314(6009): 374-7. doi: | |
| 10.1038/314374a0. PMID: | |
| 3920530 and Kaplan JM, et al. | |
| Mol Cell Biol. 1988 Jun.; | |
| 8(6): 2435-41. doi: | |
| 10.1128/mcb.8.6.2435- | |
| 2441.1988. PMID: 2841581; | |
| PMCID: PMC363442.) | |
| mut3-5′ stem | GGCACTCTTCCGTGGTCTGGTGGATAAATTCG (SEQ ID NO: |
| | 133) |
| mut3-3′ stem | CGACGTCAGACCACGGGGGAGTGCCC (SEQ ID NO: 134) |
| MS2CP | atggcttctaactttactcagttcgttctcgtcgacaatggcggaac |
| | tggcgacgtgactgtcgccccaagcaacttcgctaacggggtcgctg |
| | aatggatcagctctaactcgcgttcacaggcttacaaagtaacctgt |
| | agcgttcgtcagagctctgcgcagaatcgcaaatacaccatcaaagt |
| | cgaggtgcctaaaggcgcatggcgttcgtacttaaatatggaactaa |
| | ccattccaattttctccacgaactccgactgcgagcttattgttaag |
| | gcaatgcaaggtctcctaaaagatggaaacccgattccctcagcaat |
| | cgcagcaaactccggcatctac (SEQ ID NO: 36) |
| MS2 stem loop (stem loop that | acatgaggatcacccatgt (SEQ ID NO: 37) |
| binds the bacteriophage MS2 | |
| coat protein) | |
| MS2 | atggccagcaacttcacccagtttgtgctggtggacaatggcgggac |
| | aggcgatgtgactgtggctccctccaacttcgccaatggggtggctg |
| | agtggatcagctccaacagtcggtcacaggcctacaaggtgacctgc |
| | agcgtgcggcagtctagtgctcagaagagaaagtacacaattaaggt |
| | ggaggtgcccaaagtggccacccagacagtgggaggagtggaactgc |
| | ctgtggctgctcggagatcctacctgaacatggagctgactatccct |
| | attttcgccaccaattctgactgtgaactgatcgtgaaggctatgca |
| | gggactgctgaaagatggcaaccccatcccttctgccattgccgcta |
| | atagtggaatctat (SEQ ID NO: 135) |
| MPMV half CTE (hCTE) | CACTAACCTAAGACAGGAGGGCCGGGAAACCTGCCTAATCCAATGAC |
| | GGGTAATAGTG (SEQ ID NO: 25) |
| mHNRNPA1 M9 | AATGATTTTGGCAATTACAACAATCAGTCTTCCAATTTTGGGCCGAT |
| | GAAGGGAGGAAACTTTGGAGGCAGGAGCTCTGGCCCTTATGGTGGTG |
| | GAGGCCAGTACTTTGCTAAACCACGGAACCAAGGTGGCTAT (SEQ |
| | ID NO: 34) |
| mCherry (mammalian codon- | gtgagcaagggcgaggaggataacatggccatcatcaaggagttcat |
| optimized; monomeric | gcgcttcaaggtgcacatggagggctccgtgaacggccacgagttcg |
| derivative of DsRed fluorescent | agatcgagggcgagggcgagggccgcccctacgagggcacccagacc |
| protein; see Shaner NC, et al. | gccaagctgaaggtgaccaagggtggccccctgcccttcgcctggga |
| Nat Biotechnol. 2004 December; | catcctgtcccctcagttcatgtacggctccaaggcctacgtgaagc |
| 22(12): 1567-72. doi: | accccgccgacatccccgactacttgaagctgtccttccccgagggc |
| 10.1038/nbt1037.Epub 2004 | ttcaagtgggagcgcgtgatgaacttcgaggacggcggcgtggtgac |
| Nov. 21. PMID: 15558047) | cgtgacccaggactcctccctgcaggacggcgagttcatctacaagg |
| | tgaagctgcgcggcaccaacttcccctccgacggccccgtaatgcag |
| | aagaagaccatgggctgggaggcctcctccgagcggatgtaccccga |
| | ggacggcgccctgaagggcgagatcaagcagaggctgaagctgaagg |
| | acggcggccactacgacgctgaggtcaagaccacctacaaggccaag |
| | aagcccgtgcagctgcccggcgcctacaacgtcaacatcaagttgga |
| | catcacctcccacaacgaggactacaccatcgtggaacagtacgaac |
| | gcgccgagggccgccactccaccggcggcatggacgagctgtacaag |
| | taa (SEQ ID NO: 136) |
| KRAB(A) | gctcctgaacaacgtgaaggtgcttctcaagtttctgtttctgttac |
| | ttttgaagatgttgctgttctttttactcgtgatgaatggaaaaaac |
| | ttgatctttctcaacgttctctttatcgtgaagttatgcttgaaaat |
| | tattctaatcttgcttctatggctta (SEQ ID NO: 137) |
| KRAB of Rat Kid-1 (Q02975\| | gctcctgaacaacgtgaaggtgcttctcaagtttctgtttctgttac |
| 2-54 aa) | ttttgaagatgttgctgttctttttactcgtgatgaatggaaaaaac |
| | ttgatctttctcaacgttctctttatcgtgaagttatgcttgaaaat |
| | tattctaatcttgcttctatggcttaa (SEQ ID NO: 138) |
| Kozak sequence (see Kozak M. | TAATACGACTCACTATAGG (SEQ ID NO: 139); |
| Nucleic Acids Res. 1987 Oct. 26; | gccaccatgg (SEQ ID NO: 140); GCCACCatg |
| 15(20): 8125-48. doi: | |
| 10.1093/nar/15.20.8125. PMID: | |
| 3313277) | |
| IL2RGTC | tgccagatttgcatgcgcaactttagccgcaaaagcaccctgaccga |
| | tcatattcgcacccataccggcgaaaaaccgtttgcgtgcgatattt |
| | gcggccgcaaatttgcggcgcgcagcacccgcaccacccataccaaa |
| | attcataccggcagccagaaaccgtttcagtgccgcatttgcatgcg |
| | caactttagccgcagcgatagcctgagcaaacatattcgcacccata |
| | ccggcgaaaaaccgtttgcgtgcgatatttgcggccgcaaatttgcg |
| | cagcgcagcaacctgaaagtgcataccaaaattcatctgcgcggcag |
| | ccagctgatcgatggt (SEQ ID NO: 142) |
| Human ARC | GAGCTGGACCACCGGACCAGCGGCGGGCTCCACGCCTACCCCGGGCC |
| | GCGGGGCGGGCAGGTGGCCAAGCCCAACGTGATCCTGCAGATCGGGA |
| | AGTGCCGGGCCGAGATGCTGGAGCACGTGCGGCGGACGCACCGGCAC |
| | CTGCTGGCCGAGGTGTCCAAGCAGGTGGAGCGCGAGCTGAAGGGGCT |
| | GCACCGGTCGGTCGGGAAGCTGGAGAGCAACCTGGACGGCTACGTGC |
| | CCACGAGCGACTCGCAGCGCTGGAAGAAGTCCATCAAGGCCTGCCTG |
| | TGCCGCTGCCAGGAGACCATCGCCAACCTGGAGCGCTGGGTCAAGCG |
| | CGAGATGCACGTGTGGCGCGAGGTGTTCTACCGCCTGGAGCGCTGGG |
| | CCGACCGCCTGGAGTCCACGGGCGGCAAGTACCCGGTGGGCAGCGAG |
| | TCAGCCCGCCACACCGTTTCCGTGGGCGTGGGGGGTCCCGAGAGCTA |
| | CTGCCACGAGGCAGACGGCTACGACTACACCGTCAGCCCCTACGCCA |
| | TCACCCCGCCCCCAGCCGCTGGCGAGCTGCCCGGGCAGGAGCCCGCC |
| | GAGGCCCAGCAGTACCAGCCGTGGGTCCCCGGCGAGGACGGGCAGCC |
| | CAGCCCCGGCGTGGACACGCAGATCTTCGAGGACCCTCGAGAGTTCC |
| | TGAGCCACCTAGAGGAGTACTTGCGGCAGGTGGGCGGCTCTGAGGAG |
| | TACTGGCTGTCCCAGATCCAGAATCACATGAACGGGCCGGCCAAGAA |
| | GTGGTGGGAGTTCAAGCAGGGCTCCGTGAAGAACTGGGTGGAGTTCA |
| | AGAAGGAGTTCCTGCAGTACAGCGAGGGCACGCTGTCCCGAGAGGCC |
| | ATCCAGCGCGAGCTGGACCTGCCGCAGAAGCAGGGCGAGCCGCTGGA |
| | CCAGTTCCTGTGGCGCAAGCGGGACCTGTACCAGACGCTCTACGTGG |
| | ACGCGGACGAGGAGGAGATCATCCAGTACGTGGTGGGCACCCTGCAG |
| | CCCAAGCTCAAGCGTTTCCTGCGCCACCCCCTGCCCAAGACCCTGGA |
| | GCAGCTCATCCAGAGGGGCATGGAGGTGCAGGATGACCTGGAGCAGG |
| | CGGCCGAGCCGGCCGGCCCCCACCTCCCGGTGGAGGATGAGGCGGAG |
| | ACCCTCACGCCCGCCCCCAACAGCGAGTCCGTGGCCAGTGACCGGAC |
| | CCAGCCCGAG (SEQ ID NO: 143) |
| hSyn promoter (human | agtgcaagtgggttttaggaccaggatgaggcggggtgggggtgcct |
| synapsin I promoter; confers | acctgacgaccgaccccgacccactggacaagcacccaacccccatt |
| neuron-specific expression; see | ccccaaattgcgcatcccctatcagagagggggaggggaaacaggat |
| Kugler S, et al. Gene Ther. | gcggcgaggcgcgtgcgcactgccagcttcagcaccgcggacagtgc |
| 2003 February; 10(4): 337-47. | cttcgcccccgcctggcggcgcgcgccaccgccgcctcagcactgaa |
| doi: 10.1038/sj.gt.3301905. | ggcgcgctgacgtcactcgccggtcccccgcaaactccccttcccgg |
| PMID: 12595892) | ccaccttggtcgcgtccgcgccgccgccggcccagccggaccgcacc |
| | acgcgaggcgcgagataggggggcacgggcgcgaccatctgcgctgc |
| | ggcgccggcgactcagcgctgcctcagtctgcggtgggcagcggagg |
| | agtcgtgtcgtgcctgagagcgcag (SEQ ID NO: 30) |
| hGH poly(A) signal (human | gggtggcatccctgtgacccctccccagtgcctctcctggccctgga |
| growth hormone | agttgccactccagtgcccaccagccttgtcctaataaaattaagtt |
| polyadenylation signal) | gcatcattttgtctgactaggtgtccttctataatattatggggtgg |
| | aggggggtggtatggagcaaggggcaagttgggaagacaacctgtag |
| | ggcctgcggggtctattgggaaccaagctggagtgcagtggcacaat |
| | cttggctcactgcaatctccgcctcctgggttcaagcgattctcctg |
| | cctcagcctcccgagttgttgggattccaggcatgcatgaccaggct |
| | cagctaatttttgtttttttggtagagacggggtttcaccatattgg |
| | ccaggctggtctccaactcctaatctcaggtgatctacccaccttgg |
| | cctcccaaattgctgggattacaggcgtgaaccactgctcccttccc |
| | tgtcctt (SEQ ID NO: 144) |
| HA (human influenza | TATCCATATGATGTTCCAGATTATGCT (SEQ ID NO: 145) |
| hemagglutinin) epitope tag | |
| GPHN.FingR | atgctcgaagtcaaggaagcatcaccaaccagcatccagatcagctg |
| | gggcaagtacaaggtcatggttcgctactaccgcatcacctacggtg |
| | aaactggtggcaatagccctgtccaggaattcaccgtgcctggcagc |
| | aagtccactgctaccatcagcagcctgaaacctggtgtcgactatac |
| | catcacggtgtacgccgtcacgatcgaccactggaactaccaggacc |
| | cgatcccgatctccatcaactaccgcaccggatcc (SEQ ID NO: |
| | 27) |
| FLAG tag | gattacaaggatgacgacgataag (SEQ ID NO: 146) |
| Factor Xa site (Factor Xa | tcggccctcaat (SEQ ID NO: 147) |
| recognition and cleavage site) | |
| F30 three-way junction_part 3 | gggtcccatcattcatggcaa (SEQ ID NO: 148) |
| F30 three-way junction_part 2 | cggtccgatactctgatgat (SEQ ID NO: 149) |
| F30 three-way junction_part 1 | ttgccatgtgtatcggtccg (SEQ ID NO: 150) |
| f1 ori (right; f1 bacteriophage | ACGCGCCCTGTAGCGGCGCATTAAGCGCGGCGGGTGTGGTGGTTACG |
| origin of replication; arrow | CGCAGCGTGACCGCTACACTTGCCAGCGCCCTAGCGCCCGCTCCTTT |
| indicates direction of (+) strand | CGCTTTCTTCCCTTCCTTTCTCGCCACGTTCGCCGGCTTTCCCCGTC |
| synthesis) | AAGCTCTAAATCGGGGGCTCCCTTTAGGGTTCCGATTTAGTGCTTTA |
| | CGGCACCTCGACCCCAAAAAACTTGATTAGGGTGATGGTTCACGTAG |
| | TGGGCCATCGCCCTGATAGACGGTTTTTCGCCCTTTGACGTTGGAGT |
| | CCACGTTCTTTAATAGTGGACTCTTGTTCCAAACTGGAACAACACTC |
| | AACCCTATCTCGGTCTATTCTTTTGATTTATAAGGGATTTTGCCGAT |
| | TTCGGCCTATTGGTTAAAAAATGAGCTGATTTAACAAAAATTTAACG |
| | CGAATT (SEQ ID NO: 151) |
| E. coli RtcB | AACTATGAGCTTTTGACCACTGAGAACGCTCCTGTTAAGATGTGGAC |
| | AAAAGGCGTGCCTGTAGAGGCCGACGCTCGGCAGCAACTCATTAACA |
| | CCGCCAAGATGCCCTTTATTTTCAAGCATATTGCCGTGATGCCTGAT |
| | GTCCATCTTGGTAAGGGTTCAACAATCGGGAGCGTCATCCCTACCAA |
| | GGGTGCCATCATTCCAGCCGCCGTAGGAGTAGATATTGGATGCGGCA |
| | TGAACGCACTTAGAACAGCTCTGACCGCCGAGGATCTTCCCGAGAAC |
| | CTCGCTGAACTGCGACAGGCAATCGAGACAGCAGTTCCTCACGGCAG |
| | AACCACAGGCAGGTGTAAGAGAGATAAGGGCGCATGGGAAAACCCCC |
| | CCGTGAATGTCGACGCAAAATGGGCAGAGTTGGAAGCTGGGTATCAA |
| | TGGCTGACCCAAAAGTACCCACGGTTCCTCAATACTAATAACTATAA |
| | GCACCTTGGGACACTCGGAACCGGCAACCACTTCATAGAAATATGCC |
| | TGGACGAGTCAGATCAAGTTTGGATAATGCTCCACTCTGGTTCACGG |
| | GGCATTGGCAACGCTATAGGAACATACTTTATAGACCTGGCCCAGAA |
| | AGAGATGCAAGAAACATTGGAAACTCTCCCAAGTAGGGACCTCGCTT |
| | ACTTCATGGAGGGAACTGAGTATTTCGATGATTATCTGAAAGCCGTA |
| | GCATGGGCACAGTTGTTCGCCTCCTTGAATAGGGATGCAATGATGGA |
| | GAATGTCGTCACTGCTCTTCAAAGTATCACCCAAAAAACAGTACGCC |
| | AACCTCAGACTCTGGCAATGGAAGAGATCAACTGTCATCATAACTAC |
| | GTACAAAAGGAACAACACTTCGGCGAAGAGATCTATGTTACCCGGAA |
| | AGGGGCCGTCTCAGCTAGGGCAGGCCAATACGGCATAATCCCTGGCT |
| | CTATGGGTGCAAAAAGCTTCATAGTTCGAGGCCTTGGGAACGAGGAG |
| | AGCTTTTGTAGCTGTAGCCACGGGGCTGGTCGGGTGATGTCCCGGAC |
| | TAAAGCTAAAAAATTGTTCTCTGTTGAGGACCAAATACGGGCTACCG |
| | CACACGTAGAATGCCGGAAGGACGCCGAGGTCATCGACGAAATCCCT |
| | ATGGCCTACAAGGACATTGACGCAGTTATGGCCGCACAGTCTGACCT |
| | GGTGGAAGTTATATATACACTGAGGCAAGTAGTATGTGTGAAGGGA |
| | (SEQ ID NO: 45) |
| CMV promoter (human | GTGATGCGGTTTTGGCAGTACATCAATGGGCGTGGATAGCGGTTTGA |
| cytomegalovirus (CMV) | CTCACGGGGATTTCCAAGTCTCCACCCCATTGACGTCAATGGGAGTT |
| immediate early promoter) | TGTTTTGGCACCAAAATCAACGGGACTTTCCAAAATGTCGTAACAAC |
| | TCCGCCCCATTGACGCAAATGGGCGGTAGGCGTGTACGGTGGGAGGT |
| | CTATATAAGCAGAGCT (SEQ ID NO: 152) |
| CMV enhancer (human | GACATTGATTATTGACTAGTTATTAATAGTAATCAATTACGGGGTCA |
| cytomegalovirus immediate | TTAGTTCATAGCCCATATATGGAGTTCCGCGTTACATAACTTACGGT |
| early enhancer) | AAATGGCCCGCCTGGCTGACCGCCCAACGACCCCCGCCCATTGACGT |
| | CAATAATGACGTATGTTCCCATAGTAACGCCAATAGGGACTTTCCAT |
| | TGACGTCAATGGGTGGAGTATTTACGGTAAACTGCCCACTTGGCAGT |
| | ACATCAAGTGTATCATATGCCAAGTACGCCCCCTATTGACGTCAATG |
| | ACGGTAAATGGCCCGCCTGGCATTATGCCCAGTACATGACCTTATGG |
| | GACTTTCCTACTTGGCAGTACATCTACGTATTAGTCATCGCTATTAC |
| | CATG (SEQ ID NO: 153) |
| chicken β-actin promoter | TCGAGGTGAGCCCCACGTTCTGCTTCACTCTCCCCATCTCCCCCCCC |
| | TCCCCACCCCCAATTTTGTATTTATTTATTTTTTAATTATTTTGTGC |
| | AGCGATGGGGGCGGGGGGGGGGGGGGGGCGCGCGCCAGGCGGGGCGG |
| | GGCGGGGCGAGGGGCGGGGCGGGGCGAGGCGGAGAGGTGCGGCGGCA |
| | GCCAATCAGAGCGGCGCGCTCCGAAAGTTTCCTTTTATGGCGAGGCG |
| | GCGGCGGCGGCGGCCCTATAAAAAGCGAAGCGCGCGGCGGGCG |
| | (SEQ ID NO: 154) |
| C-farnesylation (C-far) | aagctgaaccctcctgatgagagtggccccggctgcatgagctgctg |
| | tgtgctctcctaa (SEQ ID NO: 155) |
| C-Far for membrane thethering | aagctgaaccctcctgatgagagtggccccggctgcatgagctgctg |
| | tgtgctctcctaa (SEQ ID NO: 155) |
| CCR5TC (45-159 aa) | tgccggatctgcatgcggaacttcagcgaccggtccaacctgagcag |
| | gcacatcagaacccacaccggagaaaagcccttcgcctgcgacattt |
| | gcggccggaagttcgccatcagcagcaacctgaacagccacaccaag |
| | atccacactggcagccagaaacctttccagtgcagaatttgtatgag |
| | aaactttagcagaagcgacaacctggccagacacatccggacacata |
| | ctggtgaaaaaccttttgcctgtgatatctgtggcagaaagtttgcc |
| | acctccggcaatctgacccggcacacaaagattcacctgcggggcag |
| | ccagctatcgatt (SEQ ID NO: 156) |
| CAG promoter region from pre- | TAATGATTAACCCGCCATGCTACTTATCTACGTAGCCATGCTCTAGG |
| mGRASP | AAGATCGTACCATTGACGTCAATAATGACGTATGTTCCCATAGTAAC |
| | GCCAATAGGGACTTTCCATTGACGTCAATGGGTGGACTATTTACGGT |
| | AAACTGCCCACTTGGCAGTACATCAAGTGTATCATATGCCAAGTACG |
| | CCCCCTATTGACGTCAATGACGGTAAATGGCCCGCCTGGCATTATGC |
| | CCAGTACATGACCTTATGGGACTTTCCTACTTGGCAGTACATCTACG |
| | TATTAGTCATCGCTATTACCATGGTCGAGGTGAGCCCCACGTTCTGC |
| | TTCACTCTCCCCATCTCCCCCCCCTCCCCACCCCCAATTTTGTATTT |
| | ATTTATTTTTTAATTATTTTGTGCAGCGATGGGGGGGGGGGGGGGGG |
| | GGGGGCGCGCGCCAGGCGGGGGGGGGCGGGGCGAGGGGCGGGGGGGG |
| | GCGAGGCGGAGAGGTGCGGCGGCAGCCAATCAGAGCGGCGCGCTCCG |
| | AAAGTTTCCTTTTATGGCGAGGCGGCGGCGGCGGCGGCCCTATAAAA |
| | AGCGAAGCGCGCGGCGGGCGGGAGTCGCTGCGACGCTGCCTTCGCCC |
| | CGTGCCCCGCTCCGCCGCCGCCTCGCGCCGCCCGCCCCGGCTCTGAC |
| | TGACCGCGTTACTCCCACAGGTGAGCGGGCGGGACGGCCCTTCTCCT |
| | CCGGGCTGTAATTAGCGCTTGGTTTAATGACGGCTTGTTTCTTTTCT |
| | GTGGCTGCGTGAAAGCCTTGAGGGGCTCCGGGAGGGCCCTTTGTGCG |
| | GGGGGAGCGGCTCGGGGCTGTCCGCGGGGGGACGGCTGCCTTCGGGG |
| | GGGACGGGGCAGGGCGGGGTTCGGCTTCTGGCGTGTGACCGGCGGCT |
| | CTAGAGCCTCTGCTAACCATGTTCATGCCTTCTTCTTTTTCCTACAG |
| | CTCCTGGGCAACGTGCTGGTTATTGTGCTGTCTCATCATTTTGGCAA |
| | AGAATT (SEQ ID NO: 157) |
| BoxB | GGCCCTGAAAAAGGGCC (SEQ ID NO: 20); |
| | gggccctgaagaagggccc (SEQ ID NO: 158) |
| bGH poly(A) signal (bovine | CTGTGCCTTCTAGTTGCCAGCCATCTGTTGTTTGCCCCTCCCCCGTG |
| growth hormone | CCTTCCTTGACCCTGGAAGGTGCCACTCCCACTGTCCTTTCCTAATA |
| polyadenylation signal) | AAATGAGGAAATTGCATCGCATTGTCTGAGTAGGTGTCATTCTATTC |
| | TGGGGGGTGGGGTGGGGCAGGACAGCAAGGGGGAGGATTGGGAAGAC |
| | AATAGCAGGCATGCTGGGGATGCGGTGGGCTCTATGG (SEQ ID |
| | NO: 159) |
| bc25 mer_1 | TATGAGGACGAATCTCCCGCTTATA (SEQ ID NO: 160) |
| BC200 (NR_001568) | ggccgggcgcggtggctcacgcctgtaatcccagctctcagggaggc |
| | taagaggcgggaggatagcttgagcccaggagttcgagacctgcctg |
| | ggcaatatagcgagaccccgttctccagaaaaaggaaaaaaaaaaac |
| | aaaagacaaaaaaaaaataagcgtaacttccctcaaagcaacaaccc |
| | cccccccccttt (SEQ ID NO: 19) |
| BC1 | ggggttggggatttagctcagtggtagagcgcttgcctagcaagcgc |
| | aaggccctgggttcggtcctcagctctggaaaaaaaaaaaaaaaaaa |
| | aaaaagacaaaataacaaaaagaccaaaaaaaaacaaggtaactggc |
| | acacacaaccttt (SEQ ID NO: 18) |
| Barcode sequence flanked with | CCTAGGTTAAGGATGCACCGACGGGACGTTCTATGAGGACGAATCTC |
| cloning sites | CCGCTTATAAGATTCTATAAACTGTGCGGTCCTTCAATTG (SEQ |
| | ID NO: 161) |
| Barcode insertion | GAATTCTGCAGATATC (SEQ ID NO: 162) |
| AmpR promoter (gene = bla) | ACTCTTCCTTTTTCAATATTATTGAAGCATTTATCAGGGTTATTGTC |
| | TCATGAGCGGATACATATTTGAATGTATTTAGAAAAATAAACAAATA |
| | GGGGTTCCGCG (SEQ ID NO: 163) |
| AmpR (β-lactamase, which | TTACCAATGCTTAATCAGTGAGGCACCTATCTCAGCGATCTGTCTAT |
| confers resistance to ampicillin, | TTCGTTCATCCATAGTTGCCTGACTCCCCGTCGTGTAGATAACTACG |
| carbenicillin, and related | ATACGGGAGGGCTTACCATCTGGCCCCAGTGCTGCAATGATACCGCG |
| antibiotics) | AGACCCACGCTCACCGGCTCCAGATTTATCAGCAATAAACCAGCCAG |
| | CCGGAAGGGCCGAGCGCAGAAGTGGTCCTGCAACTTTATCCGCCTCC |
| | ATCCAGTCTATTAATTGTTGCCGGGAAGCTAGAGTAAGTAGTTCGCC |
| | AGTTAATAGTTTGCGCAACGTTGTTGCCATTGCTACAGGCATCGTGG |
| | TGTCACGCTCGTCGTTTGGTATGGCTTCATTCAGCTCCGGTTCCCAA |
| | CGATCAAGGCGAGTTACATGATCCCCCATGTTGTGCAAAAAAGCGGT |
| | TAGCTCCTTCGGTCCTCCGATCGTTGTCAGAAGTAAGTTGGCCGCAG |
| | TGTTATCACTCATGGTTATGGCAGCACTGCATAATTCTCTTACTGTC |
| | ATGCCATCCGTAAGATGCTTTTCTGTGACTGGTGAGTACTCAACCAA |
| | GTCATTCTGAGAATAGTGTATGCGGCGACCGAGTTGCTCTTGCCCGG |
| | CGTCAATACGGGATAATACCGCGCCACATAGCAGAACTTTAAAAGTG |
| | CTCATCATTGGAAAACGTTCTTCGGGGCGAAAACTCTCAAGGATCTT |
| | ACCGCTGTTGAGATCCAGTTCGATGTAACCCACTCGTGCACCCAACT |
| | GATCTTCAGCATCTTTTACTTTCACCAGCGTTTCTGGGTGAGCAAAA |
| | ACAGGAAGGCAAAATGCCGCAAAAAAGGGAATAAGGGCGACACGGAA |
| | ATGTTGAATACTCAT (SEQ ID NO: 164) |
| AAV2 ITR (right; inverted | aggaacccctagtgatggagttggccactccctctctgcgcgctcgc |
| terminal repeat of adeno- | tcgctcactgaggccgggcgaccaaaggtcgcccgacgcccgggctt |
| associated virus serotype 2) | tgcccgggcggcctcagtgagcgagcgagcgcgcagctgcctgcagg |
| | (SEQ ID NO: 165) |
| AAV2 ITR (left; functional | cctgcaggcagctgcgcgctcgctcgctcactgaggccgcccgggcg |
| equivalent of wild-type AAV2 | tcgggcgacctttggtcgcccggcctcagtgagcgagcgagcgcgca |
| ITR) | gagagggagtggccaactccatcactaggggttcct (SEQ ID |
| | NO: 166) |
| A2RE (see Gao Y,et al. Mol | GCGGACGAGGA (SEQ ID NO: 167) |
| Biol Cell. 2008 May; | |
| 19(5): 2311-27. doi: | |
| 10.1091/mbc.e07-09-0914. | |
| Epub 2008 Feb. 27. PMID: | |
| 18305102; PMCID: | |
| PMC2366844) | |
| 5′P3 Twister U2A ribozyme | gccgcactcgccggtcccaagcccggataaaatggggggggcggga |
| | aaccgcct (SEQ ID NO: 168) |
| 5′ stem forming | aaccatgccgagtgcggccgc (SEQ ID NO: 169) |
| 5′ common bc25 mer 10000 | TTAAGGATGCACCGACGGGACGTTC (SEQ ID NO: 170) |
| 30A | AAAAAAAAAAAAAAAAAAAAAAAAAAAAAA (SEQ ID NO: 7) |
| 3′ stem forming | gtggccgcggtcggcgtggactgtag (SEQ ID NO: 171) |
| 3′ ribozyme | aacactgccaatgccggtcccaagcccggataaaaGTGGAGGGTACA |
| | GTCCACGC (SEQ ID NO: 172) |
| 3′ common bc25mer 10001 | AGATTCTATAAACTGTGCGGTCCTT (SEQ ID NO: 173) |

TABLE 4
Amino acid sequences for elements used in the examples.

| Element Name | Amino Acid Sequence |
|---|---|
| AmpR (β-lactamase, which confers | MSIQHFRVALIPFFAAFCLPVFAHPETLVKVKDAEDQLGARVGYI |
| resistance to ampicillin, | ELDLNSGKILESFRPEERFPMMSTFKVLLCGAVLSRIDAGQEQLG |
| carbenicillin, and related | RRIHYSQNDLVEYSPVTEKHLTDGMTVRELCSAAITMSDNTAANL |
| antibiotics) | LLTTIGGPKELTAFLHNMGDHVTRLDRWEPELNEAIPNDERDTTM |
| | PVAMATTLRKLLTGELLTLASRQQLIDWMEADKVAGPLLRSALPA |
| | GWFIADKSGAGERGSRGIIAALGPDGKPSRIVVIYTTGSQATMDE |
| | RNRQIAEIGASLIKHW (SEQ ID NO: 174) |
| CCR5TC (45-159 aa) | CRICMRNFSDRSNLSRHIRTHTGEKPFACDICGRKFAISSNLNSH |
| | TKIHTGSQKPFQCRICMRNFSRSDNLARHIRTHTGEKPFACDICG |
| | RKFATSGNLTRHTKIHLRGSQLSI (SEQ ID NO: 175) |
| C-Far for membrane thethering; C- | KLNPPDESGPGCMSCCVLS (SEQ ID NO: 23) |
| farnesylation (C-far) | |
| E. coli RtcB | NYELLTTENAPVKMWTKGVPVEADARQQLINTAKMPFIFKHIAVM |
| | PDVHLGKGSTIGSVIPTKGAIIPAAVGVDIGCGMNALRTALTAED |
| | LPENLAELRQAIETAVPHGRTTGRCKRDKGAWENPPVNVDAKWAE |
| | LEAGYQWLTQKYPRFLNTNNYKHLGTLGTGNHFIEICLDESDQVW |
| | IMLHSGSRGIGNAIGTYFIDLAQKEMQETLETLPSRDLAYFMEGT |
| | EYFDDYLKAVAWAQLFASLNRDAMMENVVTALQSITQKTVRQPQT |
| | LAMEEINCHHNYVQKEQHFGEEIYVTRKGAVSARAGQYGIIPGSM |
| | GAKSFIVRGLGNEESFCSCSHGAGRVMSRTKAKKLFSVEDQIRAT |
| | AHVECRKDAEVIDEIPMAYKDIDAVMAAQSDLVEVIYTLRQVVCV |
| | KG (SEQ ID NO: 176) |
| Factor Xa site (Factor Xa | IEGR (SEQ ID NO: 177) |
| recognition and cleavage site) | |
| FLAG tag | DYKDDDDK (SEQ ID NO: 178) |
| GPHN.FingR | MLEVKEASPTSIQISWGKYKVMVRYYRITYGETGGNSPVQEFTVP |
| | GSKSTATISSLKPGVDYTITVYAVTIDHWNYQDPIPISINYRTGS |
| | (SEQ ID NO: 26) |
| HA (human influenza | YPYDVPDYA (SEQ ID NO: 179) |
| hemagglutinin) epitope tag | |
| Human ARC | ELDHRTSGGLHAYPGPRGGQVAKPNVILQIGKCRAEMLEHVRRTH |
| | RHLLAEVSKQVERELKGLHRSVGKLESNLDGYVPTSDSQRWKKSI |
| | KACLCRCQETIANLERWVKREMHVWREVFYRLERWADRLESTGGK |
| | YPVGSESARHTVSVGVGGPESYCHEADGYDYTVSPYAITPPPAAG |
| | ELPGQEPAEAQQYQPWVPGEDGQPSPGVDTQIFEDPREFLSHLEE |
| | YLRQVGGSEEYWLSQIQNHMNGPAKKWWEFKQGSVKNWVEFKKEF |
| | LQYSEGTLSREAIQRELDLPQKQGEPLDQFLWRKRDLYQTLYVDA |
| | DEEEIIQYVVGTLQPKLKRFLRHPLPKTLEQLIQRGMEVQDDLEQ |
| | AAEPAGPHLPVEDEAETLTPAPNSESVASDRTQPE (SEQ ID |
| | NO: 180) |
| IL2RGTC | CQICMRNFSRKSTLTDHIRTHTGEKPFACDICGRKFAARSTRTTH |
| | TKIHTGSQKPFQCRICMRNFSRSDSLSKHIRTHTGEKPFACDICG |
| | RKFAQRSNLKVHTKIHLRGSQLIDG (SEQ ID NO: 181) |
| KRAB of Rat Kid-1 (Q02975\| | APEQREGASQVSVSVTFEDVAVLFTRDEWKKLDLSQRSLYREVML |
| 2-54 aa); KRAB(A) | ENYSNLASMA* (SEQ ID NO: 182) |
| mCherry (mammalian codon- | VSKGEEDNMAIIKEFMRFKVHMEGSVNGHEFEIEGEGEGRPYEGT |
| optimized; monomeric derivative | QTAKLKVTKGGPLPFAWDILSPQFMYGSKAYVKHPADIPDYLKLS |
| of DsRed fluorescent protein; see | FPEGFKWERVMNFEDGGVVTVTQDSSLQDGEFIYKVKLRGTNFPS |
| Shaner NC, et al. Nat Biotechnol. | DGPVMQKKTMGWEASSERMYPEDGALKGEIKQRLKLKDGGHYDAE |
| 2004 December; 22(12): 1567-72. | VKTTYKAKKPVQLPGAYNVNIKLDITSHNEDYTIVEQYERAEGRH |
| doi: 10.1038/nbt1037. Epub 2004 | STGGMDELYK* (SEQ ID NO: 183) |
| Nov. 21. PMID: 15558047) | |
| mHNRNPA1 M9 | NDFGNYNNQSSNFGPMKGGNFGGRSSGPYGGGGQYFAKPRNQGGY |
| | (SEQ ID NO: 33) |
| MS2 | MASNFTQFVLVDNGGTGDVTVAPSNFANGVAEWISSNSRSQAYKV |
| | TCSVRQSSAQKRKYTIKVEVPKVATQTVGGVELPVAARRSYLNME |
| | LTIPIFATNSDCELIVKAMQGLLKDGNPIPSAIAANSGIY (SEQ |
| | ID NO: 184) |
| MS2CP | MASNFTQFVLVDNGGTGDVTVAPSNFANGVAEWISSNSRSQAYKV |
| | TCSVRQSSAQNRKYTIKVEVPKGAWRSYLNMELTIPIFSTNSDCE |
| | LIVKAMQGLLKDGNPIPSAIAANSGIY (SEQ ID NO: 35) |
| myr (N-myristoylation signal from | MGSSKSKPKDPSQR (SEQ ID NO: 185) |
| Src kinase; see Pellman D, et al. | |
| Nature. 1985 Mar. 28-April 3; | |
| 314(6009): 374-7. doi: | |
| 10.1038/314374a0. PMID: | |
| 3920530 and Kaplan JM, et al. Mol | |
| Cell Biol. 1988 June; 8(6): 2435-41. | |
| doi: 10.1128/mcb.8.6.2435- | |
| 2441.1988. PMID: 2841581; | |
| PMCID: PMC363442.) | |
| NeoR/KanR (aminoglycoside | MIEQDGLHAGSPAAWVERLFGYDWAQQTIGCSDAAVFRLSAQGRP |
| phosphotransferase from Tn5, | VLFVKTDLSGALNELQDEAARLSWLATTGVPCAAVLDVVTEAGRD |
| which confers resistance to | WLLLGEVPGQDLLSSHLAPAEKVSIMADAMRRLHTLDPATCPFDH |
| neomycin, kanamycin, and G418 | QAKHRIERARTRMEAGLVDQDDLDEEHQGLAPAELFARLKARMPD |
| (Geneticin)) | GEDLVVTHGDACLPNIMVENGRFSGFIDCGRLGVADRYQDIALAT |
| | RDIAEELGGEWADRFLVLYGIAAPDSQRIAFYRLLDEFF (SEQ |
| | ID NO: 186) |
| NES (nuclear export signal from | LPPLERLTL (SEQ ID NO: 187) |
| the HIV Rev protein; see Fischer | |
| U, et al. Cell. 1995 Aug. 11; | |
| 82(3): 475-83. doi: 10.1016/ | |
| 0092-8674(95)90436-0. | |
| PMID: 7543368) | |
| N-Palmitoylation | MLCCIRRTKPVEKNEEADQELQSTVPRARDPPVAT (SEQ ID |
| | NO: 188) |
| PP7cp | SVGEATRTLTEIQSTADRQIFEEKVGPLVGRLRLTASLRQNGAKT |
| | AYRVNLKLDQADVVDCSTSVCGELPKVRYTQVWSHDVTIVANSTE |
| | ASRKSLYDLTKSLVATSQVEDLVVNLVPLGR (SEQ ID NO: |
| | 189) |
| PP7cp-1 | MSKTIVLSVGEATRTLTEIQSTADRQIFEEKVGPLVGRLRLTASL |
| | RQNGAKTAYRVNLKLDQADVVDCSTSVCGELPKVRYTQVWSHDVT |
| | IVANSTEASRKSLYDLTKSLVATSQVEDLVVNLVPLGR (SEQ |
| | ID NO: 38) |
| PP7cp-2 | SKTIVLSVGEATRTLTEIQSTADRQIFEEKVGPLVGRLRLTASLR |
| | QNGAKTAYRVNLKLDQADVVDCSTSVCGELPKVRYTQVWSHDVTI |
| | VANSTEASRKSLYDLTKSLVATSQVEDLVVNLVPLGR (SEQ ID |
| | NO: 190) |
| PSD95.FingR | MLEVKEASPTSIQISWVLHLRHVRYYRITYGETGGNSPVQEFTVP |
| | GSKSTATISGLKPGVDYTITVYAVTIFSAYRSAWPPISINYRTGT |
| | (SEQ ID NO: 42) |
| PSD95.FingR | MLEVKEASPTSIQISWVLHLRHVRYYRITYGETGGNSPVQEFTVP |
| | GSKSTATISGLKPGVDYTITVYAVTIFSAYRSAWPPISINYRTGT |
| | (SEQ ID NO: 42) |
| rat Homer1c (Q9Z214) | MGEQPIFSTRAHVFQIDPNTKKNWVPTSKHAVTVSYFYDSTRNVY |
| | RIISLDGSKAIINSTITPNMTFTKTSQKFGQWADSRANTVYGLGF |
| | SSEHHLSKFAEKFQEFKEAARLAKEKSQEKMELTSTPSQESAGGD |
| | LQSPLTPESINGTDDERTPDVTQNSEPRAEPAQNALPFSHSAGDR |
| | TQGLSHASSAISKHWEAELATLKGNNAKLTAALLESTANVKQWKQ |
| | QLAAYQEEAERLHKRVTELECVSSQANAVHSHKTELSQTVQELEE |
| | TLKVKEEEIERLKQEIDNARELQEQRDSLTQKLQEVEIRNKDLEG |
| | QLSELEQRLEKSQSEQDAFRSNLKTLLEILDGKIFELTELRDNLA |
| | KLLECS* (SEQ ID NO: 191) |
| Rat SYPH | DVVNQLVAGGQFRVVKEPLGFVKVLQWVFAIFAFATCGSYTGELR |
| | LSVECANKTESALNIEVEFEYPFRLHQVYFDAPSCVKGGTTKIFL |
| | VGDYSSSAEFFVTVAVFAFLYSMGALATYIFLQNKYRENNKGPMM |
| | DFLATAVFAFMWLVSSSAWAKGLSDVKMATDPENIIKEMPMCRQT |
| | GNTCKELRDPVTSGLNTSVVFGFLNLVLWVGNLWFVFKETGWAAP |
| | FMRAPPGAPEKQPAPGDAYGDAGYGQGPGGYGPQDSYGPQGGYQP |
| | DYGQPASGGGGYGPQGDYGQQGYGQQGAPTSFSNQM (SEQ ID |
| | NO: 192) |
| SV40 NLS | PKKKRKV (SEQ ID NO: 193) |
| T2A | EGRGSLLTCGDVEENPGP (SEQ ID NO: 194) |
| V5 tag | GKPIPNPLLGLDST (SEQ ID NO: 195) |
| VAMP2A | MSATAATVPPAAPAGEGGPPAPPPNLTSNRRLQQTQAQVDEVVDI |
| | MRVNVDKVLERDQKLSELDDRADALQAGASQFETSAAKLKRKYWW |
| | KNLKMMIILGVICAIILIIIIVYFST (SEQ ID NO: 49) |
| λ N peptide | DAQTRRRERRAEKQAQWKAAN (SEQ ID NO: 31) |

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An RNA polynucleotide comprising the following elements, each of which is operably linked:
i) a first ribozyme;
ii) a first ligation sequence;
iii) an RNA hairpin sequence;
iv) a heterologous polynucleotide;
v) a second ligation sequence; and
vi) a second ribozyme,
wherein the RNA hairpin sequence specifically binds an RNA binding polypeptide that mediates nuclear export.

2. An expression vector encoding the RNA polynucleotide of claim 1.

3. A circular RNA polynucleotide comprising an RNA hairpin sequence and a heterologous polynucleotide, wherein the RNA hairpin sequence specifically binds an RNA binding protein that mediates nuclear export.

4. A cell comprising the RNA polynucleotide of claim 1.

5. A polynucleotide encoding an RNA molecule comprising one or more of the following:
(a) from 5′ to 3′: a first ribozyme, a first ligation sequence, a PP7 RNA hairpin, a second ligation sequence, and a second ribozyme;
(b) from 5′ to 3′: first ribozyme, a first ligation sequence, a PP7 RNA hairpin, an hCTE RNA hairpin, a second ligation sequence, and a second ribozyme;
(c) from 5′ to 3′: a first ribozyme, a first ligation sequence, a BC1 RNA hairpin, a second ligation sequence, and a 3′ ribozyme; or
(d) from 5′ to 3′: a first ribozyme, a first ligation sequence, a BC200 RNA hairpin, a second ligation sequence, and a second ribozyme.

6. A polynucleotide encoding from 5′ to 3′:
(a) a first ribozyme, a first ligation sequence, a PP7 RNA hairpin, a second ligation sequence, a second ribozyme, and PP7cp fused to a Far motif,
(b) a first ribozyme, a first ligation sequence, a PP7 RNA hairpin, an hCTE RNA hairpin, a second ligation sequence, a second ribozyme, and PP7cp fused to an M9 tag and a nuclear export signal (NES);
(c) a first ribozyme, a first ligation sequence, a PP7 RNA hairpin, a second ligation sequence, a second ribozyme, and RNA 2′,3′-cyclic phosphate and 5′-OH ligase (RtcB) fused to three tandem repeats of a nuclear localization signal (NLS), a self-cleaving peptide, and PP7cp fused to a Far motif;
(d) a first ribozyme, a first ligation sequence, a PP7 RNA hairpin, a second ligation sequence, a second ribozyme, DDX39A, a self-cleaving peptide, and PP7cp fused to a Far motif;
(e) a first ribozyme, a first ligation sequence, a PP7 RNA hairpin, a second ligation sequence, a second ribozyme, and PP7cp fused to an M9 tag and a NES, a self-cleaving peptide, and PP7cp fused to a Far motif;
(f) a first ribozyme, a first ligation sequence, a PP7 RNA hairpin, an hCTE RNA hairpin, a second ligation sequence, a second ribozyme, and PP7cp fused to an M9 tag and a NES, a self-cleaving peptide, and PP7cp fused to a Far motif; or
(g) a first ribozyme, a first ligation sequence, a PP7 RNA hairpin, a second ligation sequence, a second ribozyme, and PP7cp fused to a Far motif.

7. A polynucleotide encoding from 5′ to 3′:
(a) a first ribozyme, a first ligation sequence, a PP7 RNA hairpin, a second ligation sequence, a second ribozyme, PP7cp fused to an M9 tag and a NES, a self-cleaving peptide, tdPP7cp fused VAMP2A;
(b) a first ribozyme, a first ligation sequence, a PP7 RNA hairpin, a second ligation sequence, a second ribozyme, PP7cp fused to an M9 tag and a NES, a self-cleaving peptide, SYP1 fused to tdPP7cp;
(c) a first ribozyme, a first ligation sequence, a MS2 RNA hairpin, a second ligation sequence, a second ribozyme, tandem MS2cp fused to homer1c;
(d) a first ribozyme, a first ligation sequence, a MS2 RNA hairpin, a second ligation sequence, a second ribozyme, MS2cp fused to an M9 tag and a NES, a self-cleaving peptide, a PSD95 fibronectin intrabody (FingR) polypeptide fused to tdMS2cp, CCR5TC, and KRAB;
(e) a first ribozyme, a first ligation sequence, a Box RNA hairpin, a second ligation sequence, a second ribozyme, λN fused to an M9 tag and a NES, a self-cleaving peptide, and a GPHN FingR polypeptide fused to λN, IL2RGTC, and KRAB; or
(f) a first ribozyme, a first ligation sequence, a Box RNA hairpin, a second ligation sequence, a second ribozyme, and ARC fused to λN.

8. An expression vector comprising the polynucleotide of claim 7, wherein the expression vector comprises a U6 promoter that controls expression of the RNA polynucleotide.

9. A cell comprising the polynucleotide of claim 7.

10. A system for localizing the ribozyme-assisted circular RNA molecule of claim 7 to a cellular location or a polynucleotide encoding the system, the system comprising:
(a) a circular RNA molecule comprising an RNA hairpin capable of binding an RNA binding domain and a heterologous polynucleotide; and
(b) one or more fusion proteins comprising the RNA binding domain and
(i) a polypeptide domain that localizes to a cellular location of interest; or
(ii) a nuclear export domain.

11. A method for characterizing a tissue of a subject, the method comprising:
(a) contacting a cell with the polynucleotide of claim 7 under conditions that permit expression of a circular RNA molecule encoded by the polynucleotide, wherein the circular RNA molecule comprises a unique molecular identifier;
(b) determining localization of the circular RNA molecule within the cell using spatially-resolved transcript amplicon readout mapping.

12. A method for single cell morphological tracing or characterizing viral tropism, the method comprising:
(a) contacting a cell in vivo or in vitro with a vector comprising a polynucleotide encoding one or more RNA polynucleotides and one or more RNA binding polypeptides, wherein each RNA polynucleotide comprises the following elements, each of which is operably linked:
i) a first ribozyme;
ii) a first ligation sequence;
iii) an RNA hairpin sequence;
iv) a heterologous polynucleotide comprising a unique molecular identifier;
v) a second ligation sequence; and
vi) a second ribozyme,
wherein the RNA hairpin sequence specifically binds the RNA binding polypeptides; and
wherein each RNA binding polypeptide comprises a domain that tethers the RNA binding polypeptide to a cellular membrane; and
(b) detecting the unique molecular identifier in the cell, thereby tracing single cell morphology or characterizing viral tropism.

13. A method for mapping the connectome of a neuron cell, the method comprising:
(a) contacting a neuron cell in vivo or in vitro with retrograde adenoviral associated viral (retroAAV) vector comprising a polynucleotide encoding one or more RNA polynucleotides and one or more RNA binding polypeptides, wherein each RNA polynucleotide comprises the following elements, each of which is operably linked:
i) a first ribozyme;
ii) a first ligation sequence;
iii) an RNA hairpin sequence;
iv) a heterologous polynucleotide comprising a unique molecular identifier;
v) a second ligation sequence; and
vi) a second ribozyme,
wherein the RNA hairpin sequence specifically binds the RNA binding polypeptides; and
wherein each RNA binding polypeptide comprises a domain that tethers the RNA binding polypeptide to a cellular membrane; and
(b) detecting the unique molecular identifier in the cell, thereby mapping the connectome of the neuron cell.

14. A method for introducing a heterologous polynucleotide to the cytoplasm of a cell, the method comprising (a) contacting the cell in vivo or in vitro with a vector comprising a polynucleotide encoding one or more RNA polynucleotides and an RNA binding polypeptide, wherein each RNA polynucleotide comprises the following elements, each of which is operably linked:
i) a first ribozyme;
ii) a first ligation sequence;
iii) an RNA hairpin sequence;
iv) a heterologous polynucleotide comprising a heterologous polynucleotide;
v) a second ligation sequence; and
vi) a second ribozyme,
wherein the RNA hairpin sequence specifically binds the RNA binding polypeptide; and wherein the RNA binding polypeptide mediates nuclear export.

15. A method for characterizing a tissue of a subject or characterizing viral tropism in a tissue of a subject, the method comprising:
(a) contacting an organism with an agent and a vector expressing a circular RNA barcode under conditions that permit expression of the RNA barcodes in a tissue of the subject;
(b) obtaining a biological sample from the subject and sectioning the sample to obtain tissue sections comprising expressed RNA bar codes;
(c) contacting the tissue sections with a detectable probe comprising a gene specific identifier and a region where a reading probe aligns to an endogenous gene to detect spatially resolved in situ endogenous gene sequence;
(d) contacting the tissue sections with a primer that hybridizes to a common region within the RNA barcode and a probe that hybridizes to a variable region within the RNA barcode to obtain a spatially resolved in situ RNA sequence, wherein the sequence of (c) and the sequence of (d) are computationally integrated and detected at a nanometer voxel size; and
(e) computationally analyzing the voxels to generate a molecularly defined cell-type and tissue region map comprising spatially resolved single-cell expression profile to obtain a comprehensive spatial cell atlas of the tissue or to generate a molecularly defined cell-type and tissue region map comprising spatially resolved single-cell expression profiles.

16. A method comprising:
performing in situ sequencing of each tissue section of a plurality of tissue sections of a tissue to identify genes expressed at locations within each tissue section;
identifying individual cells present within each tissue section and labeling each individual cell with a cell type using the genes identified as being expressed at the locations within each tissue section; and
storing information describing a three-dimensional structure of the tissue, the information describing the three-dimensional structure of the tissue comprising locations within the tissue at which different cell types appear.

17. A method comprising:
obtaining a reference structure for a reference sample of a tissue in a reference state, the reference structure identifying a gene expression of individual cells at locations in the reference sample of the tissue;
obtaining a second structure for a second sample of the tissue in a second state different from the reference state, the second structure identifying a gene expression of individual cells at locations in the second sample;
determining one or more differences in gene expression of individual cells between the reference state and the second state using the reference structure and the second structure; and
outputting the one or more differences in the gene expression of individual cells.

18. A method comprising:
determining information to output to a user regarding a composition of a tissue, wherein the information regarding the composition of the tissue comprises information indicating a location of individual cells within the tissue, wherein the determining comprises:
filtering a data set of information regarding the tissue responsive to user-input filtering criteria, wherein the information regarding the tissue comprises information on genes expressed in individual cells in the tissue and where the user-input filtering criteria identifies one or more genes for which information is to be output; and
selecting, for output to the user as part of the information regarding the composition of the tissue, information regarding cells detected to have expressed the one or more genes for which information is to be output, the information regarding the cells comprising the location of the cells within the tissue;
outputting the information regarding the composition of the tissue for presentation to the user.

19. An RNA polynucleotide comprising a sequence with at least 85% sequence identity to a sequence selected from the group consisting of:
a)
(SEQ ID NO: 1)
ccgcacUcgccggUcccaagcccggaUaaaaUgggagggggcgggaaaccgccUaaccaUgcc
gagUgcggccgcUUgccaUgUgUaUcggUccgacaUgaggaUcacccaUgUcggUccgaUacUc
UgaUgaU(Nₙ)gggUcccaUcaUUcaUggcaagUggccgcggUcggcgUggacUgUagaacacUg
ccaaUgccggUcccaagcccggaUaaaaGUGGAGGGUACAGUCCACGC (racRNA-MS2);
b)
(SEQ ID NO: 2)
gccgcacUcgccggUcccaagcccggaUaaaaUgggagggggcgggaaaccgccUaaccaUgc
cgagUgcggccgcUUgccaUgUgUaUcggUccgGGAGCAGACGAUAUGGCGUCGCUCCcggUcc
gaUacUcUgaUgaU(Nₙ)gggUcccaUcaUUcaUggcaagUggccgcggUcggcgUggacUgUag
aacacUgccaaUgccggUcccaagcccggaUaaaaGUGGAGGGUACAGUCCACGC
(racRNA-PP7);
c)
(SEQ ID NO: 3)
gccgcacUcgccggUcccaagcccggaUaaaaUgggagggggcgggaaaccgccUaaccaUgc
cgagUgcggccgcUUgccaUgUgUaUcggUccgcUUaagaaaaaaaaaggggUUggggaUUUag
cUcagUggUagagcgcUUgccUagcaagcgcaaggcccUgggUUcggUccUcagcUcUggaaaa
aaaaaaaaaaaaaaaaaaagacaaaaUaacaaaaagaccaaaaaaaaacaaggUaacUggcaca
cacaaccUUUaaaaaaaaagUUaaccggUccgaUacUcUgaUgaU(Nₙ)gggUcccaUcaUUcaU
ggcaagUggccgcggUcggcgUggacUgUagaacacUgccaaUgccggUcccaagcccggaUaa
aaGUGGAGGGUACAGUCCACGC (racRNA-BC1);
d)
(SEQ ID NO: 4)
cgacgggccgcacUcgccggUcccaagcccggaUaaaaUgggagggggcgggaaaccgccUaa
ccaUgccgagUgcggccgcUUgccaUgUgUaUcggUccgGGAGCAGACGAUAUGGCGUCGCUCC
cggUccgaUacUcUgaUgaU(Nₙ)CACUAACCUAAGACAGGAGGGCCGGGAAACCUGCCUAAUCC
AAUGACGGGUAAUAGUGgggacccaUcaUUcaUggcaagUggccgcggUcggcgUggacUgUag
aacacUgccaaUgccggUcccaagcccggaUaaaaGUGGAGGGUACAGUCCACGC
(racRNA-PP7-hCUE);
and
e)
(SEQ ID NO: 5)
gccgcacUcgccggUcccaagcccggaUaaaaUgggagggggcgggaaaccgccUaaccaUgc
cgagUgcggccgcUUgccaUgUgUaUcggUccgGGAGCAGACGAUAUGGCGUCGCUCCcggUcc
gaUacUcUgaUgaU(Nₙ)AAAAAAAAAAAAAAAAAAAAAAAAAAAAAAgggUcccaUcaUUcaUg
gcaagUggccgcggUcggcgUggacUgUagaacacUgccaaUgccggUcccaagcccggaUaaa
aGUGGAGGGUACAGUCCACGC (racRNA-PP7-30A);
wherein, N is any nucleotide and n is a number between 1 and 1000.

20. The RNA polynucleotide of claim 19, further comprising a polynucleotide encoding a polypeptide with at least 85% sequence identity to an amino acid sequence selected from the group consisting of:
a)
(SEQ ID NO: 8)
MASNFTQFVLVDNGGTGDVTVAPSNFANGVAEWISSNSRSQAYKVTCSVRQSSAQNRKYTIKV
EVPKGAWRSYLNMELTIPIFSTNSDCELIVKAMQGLLKDGNPIPSAIAANSGIYGGGGSGGGGS
NDFGNYNNQSSNFGPMKGGNFGGRSSGPYGGGGQYFAKPRNQGGYGGGGSLPPLERLTLGSGGS
GGSEGRGSLLTCGDVEENPGPATMLEVKEASPTSIQISWVLHLRHVRYYRITYGETGGNSPVQE
FTVPGSKSTATISGLKPGVDYTITVYAVTIFSAYRSAWPPISINYRTGTDYKDDDDKGSGSSRS
GLLKATMASNFTQFVLVDNGGTGDVTVAPSNFANGVAEWISSNSRSQAYKVTCSVRQSSAQKRK
YTIKVEVPKVATQTVGGVELPVAARRSYLNMELTIPIFATNSDCELIVKAMQGLLKDGNPIPSA
IAANSGIYGAPGIHPGMMASNFTQFVLVDNGGTGDVTVAPSNFANGVAEWISSNSRSQAYKVTC
SVRQSSAQKRKYTIKVEVPKVATQTVGGVELPVAARRSYLNMELTIPIFATNSDCELIVKAMQG
LLKDGNPIPSAIAANSGIY (MS2cp-M9-NES-T2A-PSD95.FingR-FLAG-tdMS2cp);
b)
(SEQ ID NO: 9)
MSKTIVLSVGEATRTLTEIQSTADRQIFEEKVGPLVGRLRLTASLRQNGAKTAYRVNLKLDQA
DVVDCSTSVCGELPKVRYTQVWSHDVTIVANSTEASRKSLYDLTKSLVATSQVEDLVVNLVPLG
RGGGGSGGGGSNDFGNYNNQSSNFGPMKGGNFGGRSSGPYGGGGQYFAKPRNQGGYGGGGSLPP
LERLTLGSGGSGGSEGRGSLLTCGDVEENPGPATMSKTIVLSVGEATRTLTEIQSTADRQIFEE
KVGPLVGRLRLTASLRQNGAKTAYRVNLKLDQADVVDCSTSVCGELPKVRYTQVWSHDVTIVAN
STEASRKSLYDLTKSLVATSQVEDLVVNLVPLGRRADPLASCGRSKTIVLSVGEATRTLTEIQS
TADRQIFEEKVGPLVGRLRLTASLRQNGAKTAYRVNLKLDQADVVDCSTSVCGELPKVRYTQVW
SHDVTIVANSTEASRKSLYDLTKSLVATSQVEDLVVNLVPLGRRADPLASTRDSTSGGSGGGYP
YDVPDYASGGGSGGGMSATAATVPPAAPAGEGGPPAPPPNLTSNRRLQQTQAQVDEVVDIMRVN
VDKVLERDQKLSELDDRADALQAGASQFETSAAKLKRKYWWKNLKMMIILGVICAIILIIIIVY
FST (PP7cp-M9-NES-T2A-tdPP7cp-VAMP2A);
c)
(SEQ ID NO: 10)
MGKPIPNPLLGLDSTGGGGSSKTIVLSVGEATRTLTEIQSTADRQIFEEKVGPLVGRLRLTAS
LRQNGAKTAYRVNLKLDQADVVDCSTSVCGELPKVRYTQVWSHDVTIVANSTEASRKSLYDLTK
SLVATSQVEDLVVNLVPLGRGGGGSGGGGSNDFGNYNNQSSNFGPMKGGNFGGRSSGPYGGGGQ
YFAKPRNQGGYGGGGSLPPLERLTLGSGGSGGSEGRGSLLTCGDVEENPGPAMDYKDDDDKGGG
GSSKTIVLSVGEATRTLTEIQSTADRQIFEEKVGPLVGRLRLTASLRQNGAKTAYRVNLKLDQA
DVVDCSTSVCGELPKVRYTQVWSHDVTIVANSTEASRKSLYDLTKSLVATSQVEDLVVNLVPLG
RGGGGSKLNPPDESGPGCMSCCVLS (V5-PP7cp-M9-NES-T2A-FLAG-PP7cp-Far);
d)
(SEQ ID NO: 11)
MGKPIPNPLLGLDSTGGGGSSKTIVLSVGEATRTLTEIQSTADRQIFEEKVGPLVGRLRLTAS
LRQNGAKTAYRVNLKLDQADVVDCSTSVCGELPKVRYTQVWSHDVTIVANSTEASRKSLYDLTK
SLVATSQVEDLVVNLVPLGRGGGGSGGGGSNDFGNYNNQSSNFGPMKGGNFGGRSSGPYGGGGQ
YFAKPRNQGGYGGGGSLPPLERLTLGSGGSGGSEGRGSLLTCGDVEENPGPAMVSKGEEDNMAI
IKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKL
DITSHNEDYTIVEQYERAEGRHSTGGMDELYKGGGGSSKTIVLSVGEATRTLTEIQSTADRQIF
EEKVGPLVGRLRLTASLRQNGAKTAYRVNLKLDQADVVDCSTSVCGELPKVRYTQVWSHDVTIV
ANSTEASRKSLYDLTKSLVATSQVEDLVVNLVPLGRGGGGSKLNPPDESGPGCMSCCVLS
(V5tag-PP7cp-M9-NES-T2A-mCherry-PP7cp-Far);
e)
(SEQ ID NO: 12)
VSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVK
LRGTNFPSDGPVMQKKTMGWEASSERMYPEDGALKGEIKQRLKLKDGGHYDAEVKTTYKAKKPV
QLPGAYNVNIKLDITSHNEDYTIVEQYERAEGRHSTGGMDELYK (mCherry);
f)
(SEQ ID NO: 13)
SKTIVLSVGEATRTLTEIQSTADRQIFEEKVGPLVGRLRLTASLRQNGAKTAYRVNLKLDQAD
VVDCSTSVCGELPKVRYTQVWSHDVTIVANSTEASRKSLYDLTKSLVATSQVEDLVVNLVPLGR
GGGGGGGGSNDFGNYNNQSSNFGPMKGGNFGGRSSGPYGGGGQYFAKPRNQGGYGGGGSLPPL
ERLTL (PP7cp-M9-NES);
g)
(SEQ ID NO: 14)
SKTIVLSVGEATRTLTEIQSTADRQIFEEKVGPLVGRLRLTASLRQNGAKTAYRVNLKLDQAD
VVDCSTSVCGELPKVRYTQVWSHDVTIVANSTEASRKSLYDLTKSLVATSQVEDLVVNLVPLGR
GGGGSKLNPPDESGPGCMSCCVLS (PP7cp-Far);
and
h)
(SEQ ID NO: 15)
MASNFTQFVLVDNGGTGDVTVAPSNFANGVAEWISSNSRSQAYKVTCSVRQSSAQNRKYTIKV
EVPKGAWRSYLNMELTIPIFSTNSDCELIVKAMQGLLKDGNPIPSAIAANSGIYGGGGSGGGGS
NDFGNYNNQSSNFGPMKGGNFGGRSSGPYGGGGQYFAKPRNQGGYGGGGSLPPLERLTL
(MS2cp-M9-NES).
